(19) United States
(12) Patent Application Publication
(10) Pub. No.: US 2026/0234622 A1
(43) Pub. Date: Aug. 13, 2026

(54) OLIGONUCLEOTIDE COMPOSITIONS AND METHODS THEREOF

(71) Applicant: WAVE LIFE SCIENCES LTD., Singapore (SG)

(72) Inventors: Jason Jingxin Zhang, Walpole, MA (US); Chandra Vargeese, Schwenksville, PA (US); Naoki Iwamoto, Boston, MA (US); Chikdu Shakti Shivalila, Woburn, MA (US); Nayantara Kothari, Newton, MA (US); Ann Fiegen Durbin, Shaker Heights, OH (US); Selvi Ramasamy, Wayland, MA (US); Pachamuthu Kandasamy, Lexington, MA (US); Jayakanthan Kumarasamy, Arlington, MA (US); Gopal Reddy Bommineni, Belmont, MA (US); Subramanian Marappan, Acton, MA (US); Sethumadhavan Divakaramenon, Lexington, MA (US); David Charles Donnell Butler, Medford, MA (US); Genliang Lu, Winchester, MA (US); Hailin Yang, West Roxbury, MA (US); Mamoru Shimizu, Arlington, MA (US); Prashant Monian, Arlington, MA (US); Khoa Ngoc Dang Luu, Quincy, MA (US)

(21) Appl. No.: 19/241,178

(22) Filed: Jun. 17, 2025

Related U.S. Application Data
(62) Division of application No. 17/311,285, filed on Dec. 6, 2019, now abandoned.
(60) Provisional application No. 62/776,432, filed on Dec. 6, 2018.
(60) Provisional application No. 62/916,192, filed on Oct. 16, 2019.
(60) Provisional application No. 62/916,194, filed on Oct. 16, 2019.

(30) Foreign Application Priority Data
Apr. 11, 2019 (WO) PCT/US19/27109
May 10, 2019 (WO) PCT/US19/31672

(51) Int. Cl. C12N 15/113; C12N 15/11

(52) U.S. Cl. CPC C12N 15/113; C12N 15/111; C12N 2310/315; C12N 2310/321; C12N 2310/322; C12N 2320/33

(57) ABSTRACT

Among other things, the present disclosure provides designed DMD oligonucleotides, compositions, and methods of use thereof. In some embodiments, the present disclosure provides technologies useful for repairing mutant DMD transcripts by skipping exon 51, so that the transcript can be translated into an internally truncated but at least partially functional Dystrophin protein variant. In some embodiments, the present disclosure provides technologies useful for modulating DMD transcript splicing. In some embodiments, provided technologies can alter splicing of a dystrophin (DMD) DMD transcript. In some embodiments, the present disclosure provides methods for treating diseases, such as muscular dystrophy, including but not limited to Duchenne muscular dystrophy, Becker's muscular dystrophy, etc.

### CROSS-REFERENCE TO RELATED APPLICATIONS

This application is a divisional of U.S. application Ser. No. 17/311,285, filed Jun. 4, 2021, which is a National Stage Entry of PCT Application No. PCT/US2019/065058, filed Dec. 6, 2019 and published Jun. 11, 2020 as WO 2020/118246, which claims priority to United States Provisional Application Nos. 62/776,432, filed Dec. 6, 2018, 62/916,192, filed Oct. 16, 2019, and 62/916,194, filed Oct. 16, 2019, and PCT Application Nos. PCT/US2019/027109, filed Apr. 11, 2019 and published Oct. 17, 2019 as WO 2019/200185, and PCT/US2019/031672, filed May 10, 2019 and published Nov. 14, 2019 as WO 2019/217784, the entirety of each of which is incorporated herein by reference.

### SEQUENCE LISTING

The instant application contains a Sequence Listing which has been submitted electronically in XML format and is hereby incorporated by reference in its entirety. Said XML copy, created on Dec. 15, 2025, is named 2010581-1513.xml and is 552,790 bytes in size.

### BACKGROUND

Oligonucleotides are useful in therapeutic, diagnostic, research and nanomaterials applications. The use of naturally occurring nucleic acids (e.g., unmodified DNA or RNA) for therapeutics can be limited, for example, because of their instability against extra- and intracellular nucleases and/or their poor cell penetration and distribution. There is a need for new and improved oligonucleotides and oligonucleotide compositions, such as, e.g., new oligonucleotides and oligonucleotide compositions suitable for treatment of various diseases.

### SUMMARY

Among other things, the present disclosure encompasses the recognition that structural elements of oligonucleotides, such as base sequence, chemical modifications (e.g., modifications of sugar, base, and/or internucleotidic linkages, and patterns thereof), and/or stereochemistry (e.g., stereochemistry of backbone chiral centers (chiral internucleotidic linkages), and/or patterns thereof), can have a significant impact on oligonucleotide properties, e.g., exon skipping (e.g., of exon 51 of DMD), toxicities, stability, protein binding characteristics, etc.

In some embodiments, the present disclosure provides an oligonucleotide or an oligonucleotide composition capable of mediating skipping of an exon, e.g., exon 51, of the DMD gene and useful for treating muscular dystrophy. In some embodiments, an oligonucleotide or an oligonucleotide composition is useful for treatment of muscular dystrophy. In some embodiments, an oligonucleotide or an oligonucleotide composition is a DMD oligonucleotide or DMD oligonucleotide composition that is a DMD oligonucleotide or DMD oligonucleotide composition disclosed herein (e.g., in Table A1).

In some embodiments, as demonstrated herein, provided technologies (e.g., oligonucleotides, compositions, methods, etc.) are particularly useful for reducing levels of a mutant mRNA (e.g., a DMD transcript comprising a deleterious mutation) and/or proteins encoded thereby, and increasing levels of repaired mRNA (e.g., a DMD transcript in which exon 51 is skipped to delete, correct or compensate for a deleterious mutation) and/or proteins encoded thereby.

In some embodiments, provided technologies are particularly useful for modulating splicing of DMD transcripts, e.g., to increase levels of desired splicing products and/or to reduce levels of undesired splicing products. In some embodiments, provided technologies are particularly useful for reducing levels of DMD transcripts, e.g., pre-mRNA, RNA, etc., and in many instances, reducing levels of products arising from or encoded by such DMD transcripts such as mRNA, proteins, etc. In some embodiments, a pre-mRNA or mRNA or RNA is transported from one cellular compartment (e.g., nucleus, cytoplasm, etc.) to another, and/or has been modified by one or more enzyme.

For example, in some embodiments, a Dystrophin gene can comprise an exon comprising one or more mutations associated with muscular dystrophy (including but not limited to Duchenne (Duchenne's) muscular dystrophy (DMD) and Becker (Becker's) muscular dystrophy (BMD)). In some embodiments, a disease-associated exon comprises a mutation (e.g., a missense mutation, a frameshift mutation, a nonsense mutation, a premature stop codon, etc.) in an exon. In some embodiments, the present disclosure provides compositions and methods for effectively skipping a disease-associated Dystrophin exon, while maintaining or restoring the reading frame so that a shorter (e.g., internally truncated) but partially functional dystrophin (e.g., a variant) can be produced.

Among other things, the present disclosure demonstrates that chemical modifications and/or stereochemistry can be used to modulate DMD transcript splicing by DMD oligonucleotide compositions. In some embodiments, the present disclosure provides combinations of chemical modifications and stereochemistry to improve properties of DMD oligonucleotides, e.g., their capabilities to alter splicing of DMD transcripts. In some embodiments, the present disclosure provides chirally controlled DMD oligonucleotide compositions that, when compared to a reference condition (e.g., absence of the composition, presence of a reference composition (e.g., a stereorandom composition of DMD oligonucleotides having the same constitution (as understood by those skilled in the art, unless otherwise indicated constitution generally refers to the description of the identity and connectivity (and corresponding bond multiplicities) of the atoms in a molecular entity but omitting any distinction arising from their spatial arrangement), a different chirally controlled DMD oligonucleotide composition, etc.), combinations thereof, etc.), provide increased skipping of DMD exon 51 to produce a modified (e.g., repaired) mRNA, which can be translated to produce an internally truncated but at least partially functional Dystrophin protein variant.

In some embodiments, compared to a reference condition, provided chirally controlled oligonucleotide compositions are surprisingly effective. In some embodiments, splicing of DMD exon 51 can be enhanced by more than 5, 10, 15, 20, 25, 30, 40, 50, or 100 fold.

Among other things, the present disclosure recognizes challenges of providing low toxicity oligonucleotide compositions and methods of use thereof. In some embodiments, the present disclosure provides DMD oligonucleotide compositions and methods with reduced toxicity. In some embodiments, the present disclosure provides DMD oligonucleotide compositions and methods with reduced induction of immune responses.

In some embodiments, the present disclosure provides oligonucleotides compositions (e.g., DMD oligonucleotides and compositions) with enhanced antagonism of hTLR9 activity. In some embodiments, muscular dystrophy is associated with inflammation in, e.g., muscle tissues. In some embodiments, provided technologies (e.g., DMD oligonucleotides, compositions, methods, etc.) provides both enhanced activities (e.g., exon-skipping activities) and hTLR9 antagonist activities which can be beneficial to one or more conditions and/or diseases associated with inflammation. In some embodiments, provided DMD oligonucleotides and/or compositions thereof provides both exon-skipping capabilities and decreased levels of toxicity and/or inflammation.

In some embodiments, an oligonucleotide comprises multiple internucleotidic linkages, each independently selected from various types. Various types of internucleotidic linkages differ in properties. Without wishing to be bound by any theory, the present disclosure notes that a natural phosphate linkage (phosphodiester internucleotidic linkage) is anionic and may be unstable when used by itself without other chemical modifications in vivo; a phosphorothioate internucleotidic linkage is anionic, generally more stable in vivo than a natural phosphate linkage, and may be more hydrophobic in some instances: a neutral internucleotidic linkage such as one exemplified in the present disclosure comprising a cyclic guanidine moiety is neutral at physiological pH, can be more stable in vivo than a natural phosphate linkage, and more hydrophobic.

In some embodiments, an oligonucleotide comprises a modified internucleotidic linkage which is a non-negatively charged (neutral or cationic) internucleotidic linkage in that at a pH [e.g., human physiological pH (~7.4), pH of a delivery site (e.g., an organelle, cell, tissue, organ, organism, etc.), etc.]. Without wishing to be bound by any particular theory, in at least some cases, a neutral internucleotidic linkage in an oligonucleotide can provide improved properties and/or skipping of exon 51, e.g., improved delivery, improved resistance to exonucleases and endonucleases, improved cellular uptake, improved endosomal escape and/or improved nuclear uptake, etc., compared to a comparable nucleic acid which does not comprises a neutral internucleotidic linkage.

In some embodiments, a non-negatively charged internucleotidic linkage comprises a cyclic guanidine moiety. In some embodiments, non-negatively charged internucleotidic linkage has the structure of:

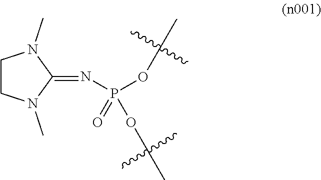 [SMILES: *OP(=O)(N=C1N(C)CCN1C)O*]

or a stereoisomer thereof (e.g., n001R or n001S). In some embodiments, a neutral internucleotidic linkage comprising a cyclic guanidine moiety is chirally controlled. In some embodiments, the present disclosure pertains to a composition comprising an oligonucleotide comprising at least one neutral internucleotidic linkage and at least one phosphorothioate internucleotidic linkage. In some embodiments, the present disclosure pertains to a composition comprising an oligonucleotide comprising at least one neutral internucleotidic linkage, at least one natural phosphate linkage, and at least one phosphorothioate internucleotidic linkage.

Among other things, the present disclosure encompasses the recognition that stereorandom DMD oligonucleotide preparations contain a plurality of distinct chemical entities that differ from one another, e.g., in the stereochemical structure of individual backbone chiral centers within the DMD oligonucleotide chain. Without control of stereochemistry of backbone chiral centers, stereorandom DMD oligonucleotide preparations provide uncontrolled (or stereorandom) compositions comprising undetermined levels of DMD oligonucleotide stereoisomers. Even though these stereoisomers may have the same base sequence and/or chemical modifications, they are different chemical entities at least due to their different backbone stereochemistry, and they can have, as demonstrated herein, different properties, e.g., skipping of exon 51, toxicities, distribution etc. Among other things, the present disclosure provides chirally controlled compositions that are or contain particular stereoisomers of DMD oligonucleotides of interest; in contrast to chirally uncontrolled compositions, chirally controlled compositions comprise controlled levels of particular stereoisomers of DMD oligonucleotides. In some embodiments, a particular stereoisomer may be defined, for example, by its base sequence, its pattern of backbone linkages, its pattern of backbone chiral centers, and pattern of backbone phosphorus modifications, etc. As is understood in the art, in some embodiments, base sequence may refer solely to the sequence of bases and/or to the identity and/or modification status of nucleoside residues (e.g., of sugar and/or base components, relative to standard naturally occurring nucleotides such as adenine, cytosine, guanosine, thymine, and uracil) in a DMD oligonucleotide and/or to the hybridization character (i.e., the ability to hybridize with particular complementary residues) of such residues. In some embodiments, the present disclosure demonstrates that property improvements (e.g., improved skipping of exon 51, lower toxicities, etc.) achieved through inclusion and/or location of particular chiral structures within a DMD oligonucleotide can be comparable to, or even better than those achieved through use of chemical modifications, e.g., particular backbone linkages, residue modifications, etc. (e.g., through use of certain types of modified phosphates [e.g., phosphorothioate, substituted phosphorothioate, etc.], sugar modifications [e.g., 2′-modifications, etc.], and/or base modifications [e.g., methylation, etc.]). In some embodiments, the present disclosure demonstrates that chirally controlled DMD oligonucleotide compositions of DMD oligonucleotides comprising certain chemical modifications (e.g., 2′-F, 2′-OMe, phosphorothioate internucleotidic linkages, etc.) demonstrate unexpectedly high exon-skipping efficiency.

In some embodiments, the present disclosure provides a DMD oligonucleotide composition comprising a plurality of DMD oligonucleotides which:
1) have a common base sequence complementary to a target sequence in a DMD transcript; and
2) comprise one or more modified sugar moieties and modified internucleotidic linkages, wherein the DMD oligonucleotide is a DMD oligonucleotide described herein (e.g., in Table A1).

In some embodiments, a provided DMD oligonucleotide composition is characterized in that, when it is contacted with a DMD transcript in a DMD transcript splicing system, splicing of the DMD transcript is altered (e.g., skipping of exon 51 is increased) relative to that observed under a reference condition selected from the group consisting of absence of the composition, presence of a reference composition, and combinations thereof.

In some embodiments, a reference condition is absence of the composition. In some embodiments, a reference condition is presence of a reference composition. Example reference compositions comprising a reference plurality of DMD oligonucleotides are extensively described in this disclosure. In some embodiments, DMD oligonucleotides of the reference plurality have a different structural elements (chemical modifications, stereochemistry, etc.) compared with DMD oligonucleotides of the plurality in a provided composition. In some embodiments, a reference composition is a stereorandom preparation of DMD oligonucleotides having the same chemical modifications. In some embodiments, a reference composition is a mixture of stereoisomers while a provided composition is a chirally controlled DMD oligonucleotide composition of one stereoisomer. In some embodiments, DMD oligonucleotides of the reference plurality have the same base sequence, same sugar modifications, same base modifications, same internucleotidic linkage modifications, and/or same stereochemistry as DMD oligonucleotide of the plurality in a provided composition but different chemical modifications, e.g., base modification, sugar modification, internucleotidic linkage modifications, etc.

Example splicing systems are widely known in the art. In some embodiments, a splicing system is an in vivo or in vitro system including components sufficient to achieve splicing of a relevant target DMD transcript. In some embodiments, a splicing system is or comprises a spliceosome (e.g., protein and/or RNA components thereof). In some embodiments, a splicing system is or comprises an organellar membrane (e.g., a nuclear membrane) and/or an organelle (e.g., a nucleus). In some embodiments, a splicing system is or comprises a cell or population thereof. In some embodiments, a splicing system is or comprises a tissue. In some embodiments, a splicing system is or comprises an organism, e.g., an animal, e.g., a mammal such as a mouse, rat, monkey, dog, human, etc.

In some embodiments, the present disclosure provides a DMD oligonucleotide composition comprising a plurality of DMD oligonucleotides of a particular DMD oligonucleotide type defined by:
1) base sequence;
2) pattern of backbone linkages:
3) pattern of backbone chiral centers; and
4) pattern of backbone phosphorus modifications, wherein the DMD oligonucleotide is a DMD oligonucleotide described herein (e.g., in Table A1).

In some embodiments, the present disclosure provides a DMD oligonucleotide composition comprising a plurality of DMD oligonucleotides of a particular DMD oligonucleotide type defined by:
1) base sequence;
2) pattern of backbone linkages:
3) pattern of backbone chiral centers; and
4) pattern of backbone phosphorus modifications,
   which composition is chirally controlled and it is enriched, relative to a substantially racemic preparation of DMD oligonucleotides having the same base sequence, for DMD oligonucleotides of the particular DMD oligonucleotide type,
the DMD oligonucleotide composition being characterized in that, when it is contacted with the DMD transcript in a DMD transcript splicing system, splicing of the DMD transcript is altered (e.g., skipping of exon 51 is increased) relative to that observed under reference conditions selected from the group consisting of absence of the composition, presence of a reference composition, and combinations thereof, wherein the DMD oligonucleotide is a DMD oligonucleotide described herein (e.g., in Table A1).

In some embodiments, the present disclosure provides a chirally controlled oligonucleotide composition of an oligonucleotide in Table A1, wherein the oligonucleotide comprises one or more (e.g., 1, 2, 3, 4, 5, 6, 7, 8, 9, 10, 11, 12, 13, 14, 15, 16, 17, 18, 19, or 20 or more) chirally controlled internucleotidic linkages (e.g., those of S, R, nS, or nR), and wherein the oligonucleotide is optionally in a pharmaceutically acceptable salt form. In some embodiments, the oligonucleotide is provided as a sodium salt.

In some embodiments, as described herein a plurality of oligonucleotides share the same constitution. In some embodiments, for a chirally controlled internucleotidic linkage of a plurality of oligonucleotides in a composition, at least 85%, 90%, 91%, 92%, 93%, 94%, 95%, 96%, 97%, 98%, or 99%, preferably at least 90%, 91%, 92%, 93%, 94%, 95%, 96%, 97%, 98%, or 99%, of all oligonucleotides in the composition that share the same constitution of the plurality of the oligonucleotides share the same linkage phosphorus configuration at the chirally controlled internucleotidic linkage.

In some embodiments, a DMD transcript is of a Dystrophin gene or a variant thereof.

In some embodiments, the present disclosure provides a composition comprising any DMD oligonucleotide disclosed herein. In some embodiments, the present disclosure provides a composition comprising any chirally controlled DMD oligonucleotide disclosed herein. In some embodiments, the present disclosure provides a composition comprising any chirally controlled DMD oligonucleotide disclosed herein, wherein the DMD oligonucleotide is capable of mediating skipping of DMD exon 51.

In some embodiments, the present disclosure pertains to any individual DMD oligonucleotide described herein (e.g., in Table A1).

In some embodiments, a provided DMD oligonucleotide and/or composition is capable of mediating skipping of exon 51. In some embodiments, non-limiting examples of such DMD oligonucleotides and compositions include those of: WV-20011, WV-20052, WV-20059, WV-20072, WV-20073, WV-20074, WV-20075, WV-20076, WV-20096, WV-20097, WV-20101, and WV-20119, and other DMD oligonucleotides having a base sequence which comprises at least 15 contiguous bases of any of these DMD oligonucleotides.

In some embodiments, the present disclosure pertains to the DMD oligonucleotide or an oligonucleotide composition comprising: WV-20011, or a method of use thereof.

In some embodiments, the present disclosure pertains to the DMD oligonucleotide or an oligonucleotide composition comprising: WV-20052, or a method of use thereof.

In some embodiments, the present disclosure pertains to the DMD oligonucleotide or an oligonucleotide composition comprising: WV-20059, or a method of use thereof.

In some embodiments, the present disclosure pertains to the DMD oligonucleotide or an oligonucleotide composition comprising: WV-20072, or a method of use thereof.

In some embodiments, the present disclosure pertains to the DMD oligonucleotide or an oligonucleotide composition comprising: WV-20073, or a method of use thereof.

In some embodiments, the present disclosure pertains to the DMD oligonucleotide or an oligonucleotide composition comprising: WV-20074, or a method of use thereof.

In some embodiments, the present disclosure pertains to the DMD oligonucleotide or an oligonucleotide composition comprising: WV-20075, or a method of use thereof.

In some embodiments, the present disclosure pertains to the DMD oligonucleotide or an oligonucleotide composition comprising: WV-20076, or a method of use thereof.

In some embodiments, the present disclosure pertains to the DMD oligonucleotide or an oligonucleotide composition comprising: WV-20096, or a method of use thereof.

In some embodiments, the present disclosure pertains to the DMD oligonucleotide or an oligonucleotide composition comprising: WV-20097, or a method of use thereof.

In some embodiments, the present disclosure pertains to the DMD oligonucleotide or an oligonucleotide composition comprising: WV-20101, or a method of use thereof.

In some embodiments, the present disclosure pertains to the DMD oligonucleotide or an oligonucleotide composition comprising: WV-20119, or a method of use thereof.

In some embodiments, the present disclosure pertains to a method of manufacturing any DMD oligonucleotide disclosed herein (e.g., in Table A1).

In some embodiments, the present disclosure pertains to a medicament comprising any DMD oligonucleotide disclosed herein (e.g., in Table A1).

In some embodiments, in an oligonucleotide sequence herein (including but not limited to, in Table A1): If a sugar is not specified, the sugar is a natural DNA sugar, and if an internucleotidic linkage is not specified, the internucleotidic linkage is a natural phosphate linkage.

In some embodiments, the present disclosure provides a chirally controlled DMD oligonucleotide composition of a DMD oligonucleotide selected from any of the Tables.

In some embodiments, a DMD oligonucleotide comprises an internucleotidic linkage which is a natural phosphate linkage or a phosphorothioate internucleotidic linkage. In some embodiments, a phosphorothioate internucleotidic linkage is not chirally controlled. In some embodiments, a phosphorothioate internucleotidic linkage is a chirally controlled internucleotidic linkage (e.g., Sp or Rp).

In some embodiments, a DMD oligonucleotide comprises a non-negatively charged internucleotidic linkage. In some embodiments, a DMD oligonucleotide comprises a neutral internucleotidic linkage. In some embodiments, a neutral internucleotidic linkage is or comprises a cyclic guanidine moiety.

In some embodiments, an internucleotidic linkage comprises a guanidine moiety. In some embodiments, an internucleotidic linkage comprises a cyclic guanidine moiety. In some embodiments, an internucleotidic linkage comprising a cyclic guanidine moiety has the structure of: n001. In some embodiments, a neutral internucleotidic linkage or internucleotidic linkage comprising a cyclic guanidine moiety is stereochemically controlled.

In general, properties of DMD oligonucleotide compositions as described herein can be assessed using any appropriate assay. Relative toxicity and/or protein binding properties for different compositions (e.g., stereocontrolled vs non-stereocontrolled, and/or different stereocontrolled compositions) are typically desirably determined in the same assay, in some embodiments substantially simultaneously and in some embodiments with reference to historical results.

Those of skill in the art will be aware of and/or will readily be able to develop appropriate assays for particular DMD oligonucleotide compositions. The present disclosure provides descriptions of certain particular assays, for example that may be useful in assessing one or more features of DMD oligonucleotide composition behavior e.g., complement activation, injection site inflammation, protein biding, etc.

For example, certain assays that may be useful in the assessment of toxicity and/or protein binding properties of DMD oligonucleotide compositions may include any assay described and/or exemplified herein.

In some embodiments, the present disclosure provides a DMD oligonucleotide composition comprising a plurality of DMD oligonucleotides which share the same base sequence, wherein oligonucleotides of the plurality comprise at least 1, 2, 3, 4, 5, 6, 7, 8, 9, 10, 11, 12, 13, 14, 15, 16, 17, 18, 19, or 20 chirally controlled internucleotidic linkages. In some embodiments, the present disclosure provides a DMD oligonucleotide composition comprising a plurality of DMD oligonucleotides which share the same constitution, wherein oligonucleotides of the plurality comprise at least 1, 2, 3, 4, 5, 6, 7, 8, 9, 10, 11, 12, 13, 14, 15, 16, 17, 18, 19, or 20 chirally controlled internucleotidic linkages. In some embodiments, when an oligonucleotide compositions is contacted with a DMD transcript in a DMD transcript splicing system, splicing of the DMD transcript is altered (e.g., skipping of exon 51 is increased) relative to that observed under a reference condition selected from the group consisting of absence of the composition, presence of a reference composition, and combinations thereof. In some embodiments, splicing products with one exon skipped (e.g., in some embodiments, exon 51) and/or proteins encoded thereby are provided at an increased level (e.g., 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500 or more fold) compared to a reference condition.

In some embodiments, the present disclosure provides a DMD oligonucleotide composition, comprising a plurality of DMD oligonucleotides of a particular DMD oligonucleotide type defined by:
1) base sequence;
2) pattern of backbone linkages;
3) pattern of backbone chiral centers; and
4) pattern of backbone phosphorus modifications,
   wherein:
oligonucleotides of the plurality comprise at least 1, 2, 3, 4, 5, 6, 7, 8, 9, 10, 11, 12, 13, 14, 15, 16, 17, 18, 19, or 20 chirally controlled internucleotidic linkages; and
the DMD oligonucleotide composition being characterized in that, when it is contacted with a DMD transcript in a DMD transcript splicing system, splicing of the DMD transcript is altered (e.g., skipping of exon 51 is increased) relative to that observed under a reference condition selected from the group consisting of absence of the composition, presence of a reference composition, and combinations thereof.

In some embodiments, the present disclosure provides a method for treating or preventing muscular dystrophy, comprising administering to a subject a DMD oligonucleotide composition described herein.

In some embodiments, the present disclosure provides a method for treating or preventing muscular dystrophy, comprising administering to a subject a DMD oligonucleotide composition comprising a plurality of DMD oligonucleotides, which:
1) have a common base sequence complementary to a target sequence in a DMD transcript; and
2) comprise one or more modified sugar moieties and modified internucleotidic linkages,
the DMD oligonucleotide composition being characterized in that, when it is contacted with the DMD transcript in a DMD transcript splicing system, splicing of the DMD transcript is altered (e.g., skipping of exon 51 is increased) relative to that observed under reference conditions selected from the group consisting of absence of the composition, presence of a reference composition, and combinations thereof, wherein the DMD oligonucleotide is a DMD oligonucleotide described herein (e.g., in Table A1).

In some embodiments, the present disclosure provides a method for treating or preventing muscular dystrophy, comprising administering to a subject a chirally controlled DMD oligonucleotide composition comprising a plurality of DMD oligonucleotides of a particular DMD oligonucleotide type defined by:
1) base sequence.
2) pattern of backbone linkages;
3) pattern of backbone chiral centers; and
4) pattern of backbone phosphorus modifications,
   which composition is chirally controlled and it is enriched, relative to a substantially racemic preparation of DMD oligonucleotides having the same base sequence, for DMD oligonucleotides of the particular DMD oligonucleotide type, wherein:
the DMD oligonucleotide composition being characterized in that, when it is contacted with the DMD transcript in a DMD transcript splicing system, splicing of the DMD transcript is altered (e.g., skipping of exon 51 is increased) relative to that observed under reference conditions selected from the group consisting of absence of the composition, presence of a reference composition, and combinations thereof, wherein the DMD oligonucleotide is a DMD oligonucleotide described herein (e.g., in Table A1).

In some embodiments, provided oligonucleotides comprise at least one, e.g., 1, 2, 3, 4, 5, 6, 7, 8, 9, 10, 11, 12, 13, 14, 15, 16, 17, 18, 19, 20 non-negatively charged internucleotidic linkages, which are optionally and independently chirally controlled. In some embodiments, a provided oligonucleotide comprises a chirally controlled non-negatively charged internucleotidic linkage. In some embodiments, a non-negatively charged internucleotidic linkage is n001.

In some embodiments, the present disclosure provides a DMD oligonucleotide composition, comprising a plurality of DMD oligonucleotides of a particular DMD oligonucleotide type defined by:
1) base sequence;
2) pattern of backbone linkages:
3) pattern of backbone chiral centers; and
4) pattern of backbone phosphorus modifications,
   wherein:
oligonucleotides of the plurality comprise at least 1, 2, 3, 4, 5, 6, 7, 8, 9, 10, 11, 12, 13, 14, 15, 16, 17, 18, 19, or 20 chirally controlled internucleotidic linkages; and
oligonucleotides of the plurality comprise at least 1, 2, 3, 4, 5, 6, 7, 8, 9, 10, 11, 12, 13, 14, 15, 16, 17, 18, 19, or 20 non-negatively charged internucleotidic linkages.

In some embodiments, in a muscular dystrophy, after skipping DMD exon 51, functions of dystrophin can be restored, or at least partially restored, through an internally truncated but at least partially functional Dystrophin protein variant.

In some embodiments, a muscular dystrophy includes but is not limited to Duchenne (Duchenne's) muscular dystrophy (DMD) and Becker (Becker's) muscular dystrophy (BMD).

In some embodiments, the present disclosure provides a pharmaceutical composition comprising a DMD oligonucleotide or a DMD oligonucleotide composition of the present disclosure and a pharmaceutically acceptable carrier.

In some embodiments, the present disclosure provides a method for treating muscular dystrophy, Duchenne (Duchenne's) muscular dystrophy (DMD), or Becker (Becker's) muscular dystrophy (BMD), comprising administering to a subject susceptible thereto or suffering therefrom a composition described in the present disclosure.

In some embodiments, the present disclosure provides a method for treating muscular dystrophy, Duchenne (Duchenne's) muscular dystrophy (DMD), or Becker (Becker's) muscular dystrophy (BMD), comprising administering to a subject susceptible thereto or suffering therefrom a composition comprising any DMD oligonucleotide disclosed herein. In some embodiments, a composition is a pharmaceutical composition comprising an effective amount of an oligonucleotide and is chirally controlled. In some embodiments, an oligonucleotide is provided as a salt form, e.g., a sodium salt.

In some embodiments, the present disclosure provides a method for treating muscular dystrophy, Duchenne (Duchenne's) muscular dystrophy (DMD), or Becker (Becker's) muscular dystrophy (BMD), comprising (a) administering to a subject susceptible thereto or suffering therefrom a composition comprising any DMD oligonucleotide disclosed herein, and (b) administering to the subject an additional treatment which is capable of preventing, treating, ameliorating or slowing the progress of at least one symptom of muscular dystrophy, Duchenne (Duchenne's) muscular dystrophy (DMD), or Becker (Becker's) muscular dystrophy (BMD).

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons. New York: 2001.

Aliphatic: The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic or polycyclic hydrocarbon that is completely saturated or that contains one or more units of unsaturation, but which is not aromatic (also referred to herein as “carbocycle” “cycloaliphatic” or “cycloalkyl”), or combinations thereof. In some embodiments, aliphatic groups contain 1-100 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. In some embodiments, “cycloaliphatic” (or “carbocycle” or “cycloalkyl”) refers to a monocyclic or bicyclic or polycyclic hydrocarbon that is completely saturated or that contains one or more units of unsaturation, but which is not aromatic. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, an alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 14 carbon atoms (e.g., C₁-C₄ for straight chain lower alkyls).

Alkynyl: As used herein, the term “alkynyl” refers to an aliphatic group, as defined herein, having one or more triple bonds.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of, e.g., five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but not limited to, phenyl, biphenyl, naphthyl, binaphthyl, anthracyl and the like, which may bear one or more substituents. Also included within the scope of the term “aryl.” as it is used herein, is an aromatic ring fused to one or more non-aromatic rings, such as indanyl, phthalimidyl, naphthimidyl, phenanthridinyl, or tetrahydronaphthyl, and the like.

Comparable: The term “comparable” is used herein to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or 1,2,3,4-tetrahydronaphth-1-yl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, or a C₉-C₁₆ polycyclic hydrocarbon that is completely saturated or that contains one or more units of unsaturation, but which is not aromatic.

Dosing regimen: As used herein, a “dosing regimen” or “therapeutic regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regime comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount.

Heteroaliphatic: The term “heteroaliphatic” refers to an aliphatic group wherein one or more units selected from C, CH, CH₂, and CH₃ are independently replaced by one or more heteroatoms. In some embodiments, a heteroaliphatic group is heteroalkyl. In some embodiments, a heteroaliphatic group is heteroalkenyl.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of, e.g., five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom” means an atom that is not carbon or hydrogen. In some embodiments, a heteroatom is oxygen, sulfur, nitrogen, phosphorus, boron or silicon (including any oxidized form of nitrogen, sulfur, phosphorus, or silicon; the quaternized form of any basic nitrogen or a substitutable nitrogen of a heterocyclic ring (for example, N as in 3,4-dihydro-2H-pyrrolyl), NH (as in pyrrolidinyl) or NR⁺ (as in N-substituted pyrrolidinyl); etc.). In some embodiments, a heteroatom is boron, nitrogen, oxygen, silicon, sulfur, or phosphorus. In some embodiments, a heteroatom is nitrogen, oxygen, silicon, sulfur, or phosphorus. In some embodiments, a heteroatom is nitrogen, oxygen, sulfur, or phosphorus. In some embodiments, a heteroatom is nitrogen, oxygen or sulfur.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include heterocyclyl rings fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

In vitro: As used herein, the term “in vitro” refers to events that occur in an artificial environment, e.g., in a test tube or reaction vessel, in cell culture, etc., rather than within an organism (e.g., animal, plant, and/or microbe).

In vivo: As used herein, the term “in vivo” refers to events that occur within an organism (e.g., animal, plant, and/or microbe).

Optionally substituted: As described herein, compounds of the disclosure, e.g., oligonucleotides, lipids, carbohydrates, etc., may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents are halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂: —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)N(R^{∘})₂; —N(R^{∘})C(S)N(R^{∘})₂; —(CH₂)₀₋₄N(R^{∘})C(O)OR^{∘}; —N(R^{∘})N(R^{∘})C(O)R^{∘}; —N(R^{∘})N(R^{∘})C(O)N(R^{∘})₂; —N(R^{∘})N(R^{∘})C(O)OR^{∘}; —(CH₂)₀₋₄ C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSi(R^{∘})₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)N(R^{∘})₂; —C(S)N(R^{∘})₂; —C(S)SR^{∘}; —SC(S)SR^{∘}, —(CH₂)₀₋₄OC(O)N(R^{∘})₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂N(R^{∘})₂; —(CH₂)₀₋₄S(O)R^{∘}; —N(R^{∘})S(O)₂N(R^{∘})₂; —N(R^{∘})S(O)₂R^{∘}; —N(OR^{∘})R^{∘}; —C(NH)N(R^{∘})₂; —Si(R^{∘})₃; —OSi(R^{∘})₃; —P(R^{∘})₂; —P(OR^{∘})₂: —P(R^{∘})(OR^{∘}); —OP(R^{∘})₂; —OP(OR^{∘})₂; —OP(R^{∘})(OR^{∘}); —P[N(R^{∘})₂]₂—P(R^{∘})[N(R^{∘})₂]; —P(OR^{∘})[N(R^{∘})₂]; —OP[N(R^{∘})₂]₂: —OP(R^{∘})[N(R^{∘})₂]; —OP(OR^{∘})[N(R^{∘})₂]; —N(R^{∘})P(R^{∘})₂; —N(R^{∘})P(OR^{∘})₂: —N(R^{∘})P(R^{∘})(OR^{∘}); —N(R^{∘})P[N(R^{∘})₂]₂; —N(R^{∘})P(R^{∘})[N(R^{∘})₂]; —N(R^{∘})P(OR^{∘})[N(R^{∘})₂]; —B(R^{∘})₂; —B(R^{∘})(OR^{∘}); —B(OR^{∘})₂; —OB(R^{∘})₂; —OB(R^{∘})(OR^{∘}); —OB(OR^{∘})₂; —P(O)(R^{∘})₂; —P(O)(R^{∘})(OR^{∘}); —P(O)(R^{∘})(SR^{∘}); —P(O)(R^{∘})[N(R^{∘})₂]; —P(O)(OR^{∘})₂; —P(O)(SR^{∘})₂; —P(O)(OR^{∘})[N(R^{∘})₂]; —P(O)(SR^{∘})[N(R^{∘})₂]; —P(O)(OR^{∘})(SR^{∘}); —P(O)[N(R^{∘})₂]₂; —OP(O)(R^{∘})₂; —OP(O)(R^{∘})(OR^{∘}); —OP(O)(R^{∘})(SR^{∘}); —OP(O)(R^{∘})[N(R^{∘})₂]; —OP(O)(OR^{∘})₂; —OP(O)(SR^{∘})₂; —OP(O)(OR^{∘})[N(R^{∘})₂]; —OP(O)(SR^{∘})[N(R^{∘})₂]; —OP(O)(OR^{∘})(SR^{∘}); —OP(O)[N(R^{∘})₂]2; —SP(O)(R^{∘})₂; —SP(O)(R^{∘})(OR^{∘}); —SP(O)(R^{∘})(SR^{∘}): —SP(O)(R^{∘})[N(R^{∘})₂]; —SP(O)(OR^{∘})₂; —SP(O)(SR^{∘})₂; —SP(O)(OR^{∘})[N(R^{∘})₂]; —SP(O)(SR^{∘})[N(R^{∘})₂]; —SP(O)(OR^{∘})(SR^{∘}); —SP(O)[N(R^{∘})₂]₂; —N(R^{∘})P(O)(R^{∘})₂; —N(R^{∘})P(O)(R^{∘})(OR^{∘}); —N(R^{∘})P(O)(R^{∘})(SR^{∘}); —N(R^{∘})P(O)(R^{∘})[N(R^{∘})₂]; —N(R^{∘})P(O)(OR^{∘})₂; —N(R^{∘})P(O)(SR^{∘})₂; —N(R^{∘})P(O)(OR^{∘})[N(R^{∘})₂]; —N(R^{∘})P(O)(SR^{∘})[N(R^{∘})₂]; —N(R^{∘})P(O)(OR^{∘})(SR^{∘}); —N(R^{∘})P(O)[N(R^{∘})₂]₂; —P(R^{∘})₂][B(R^{∘})₃]; —P(OR^{∘})₂][B(R^{∘})₃]; —P(NR^{∘})₂[B(R^{∘})₃]; —P(R^{∘})(OR^{∘})[B(R^{∘})₃]; —P(R^{∘})[N(R^{∘})₂][B(R^{∘})₃]; —P(OR^{∘})[N(R^{∘})₂][B(R^{∘})₃]; —OP(R^{∘})₂[B(R^{∘})₃]; —OP(OR^{∘})₂[B(R^{∘})₃]; —OP(NR^{∘})₂[B(R^{∘})₃]; —OP(R^{∘})(OR^{∘})[B(R^{∘})₃]; —OP(R^{∘})[N(R^{∘})₂][B(R^{∘})₃]; —OP(OR^{∘})[N(R^{∘})₂][B(R^{∘})₃]; —N(R^{∘})P(R^{∘})₂[B(R^{∘})₃]; —N(R^{∘})P(OR^{∘})₂[B(R^{∘})₃]; —N(R^{∘})P(NR^{∘})₂[B(R^{∘})₃]; —N(R^{∘})P(R^{∘})(OR^{∘})[B(R^{∘})₃]; —N(R^{∘})P(R^{∘})[N(R^{∘})₂][B(R^{∘})₃]; —N(R^{∘})P(OR^{∘})[N(R^{∘})₂][B(R^{∘})₃]; —P(OR^{∘})[B(R′)₃]—; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₂₀ aryl), —O(CH₂)₀₋₁(C₆₋₂₀ aryl), —CH₂-(5-20 membered heteroaryl ring having 1-5 heteroatoms independently selected from nitrogen, oxygen, sulfur, silicon and phosphorus), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{o}, taken together with their intervening atom(s), form a 3-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph. —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents, e.g., on a suitable carbon atom, nitrogen atom, are independently the following: ═O, ═S, ═CR*₂, ═NNR*₂, ═NNHC(O)R*, ═NNHC(O)OR*, ═NNHS(O)₂R*, β2 NR*, ═NOR*, —O(C(R*₂))₂₋₃O—, or —S(C(R*₂))₂₋₃S—, wherein each R* may be substituted as defined below and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₂₀ aryl), —O(CH₂)₀₋₁(C₆₋₂₀ aryl), —CH₂-(5-20 membered heteroaryl ring having 1-5 heteroatoms independently selected from nitrogen, oxygen, sulfur, silicon and phosphorus), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R*, taken together with their intervening atom(s), form a 3-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below. Suitable divalent substituents that are bound to vicinal substitutable atoms of an “optionally substituted” group include: —O(CR*₂)₂₋₃O—.

Suitable monovalent substituents on R* (or the ring formed by taking two independent occurrences of R* together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R* include ═O and ═S.

In some embodiments, suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}2, or —N(R^{†})S(O)₂R^{†}; wherein each R^{†} is independently hydrogen, C₁₋₆ aliphatic which may be substituted as defined below, unsubstituted —OPh, or an unsubstituted 5-6 membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12 membered saturated, partially unsaturated, or aryl mono- or bicyclic ring having 0-4 heteroatoms independently selected from nitrogen, oxygen, or sulfur.

In some embodiments, suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6 membered saturated, partially unsaturated, or aryl ring having 0-4 heteroatoms independently selected from nitrogen, oxygen, or sulfur.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a controlled therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets. e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity: intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes: oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide, alginic acid; pyrogen-free water: isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3 -phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound, e.g., an oligonucleotide, comprises one or more acidic groups (e.g., natural phosphate linkage groups, phosphorothioate linkage groups, etc.) and a pharmaceutically acceptable salt is an alkali, alkaline earth metal, or ammonium (e.g., an ammonium salt of N(R)₃, wherein each R is independently as defined and described in the present disclosure) salt. Representative alkali or alkaline earth metal salts include salts of sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups, for example, an oligonucleotide may comprise two or more acidic groups (e.g., in natural phosphate linkages and/or modified internucleotidic linkages). In some embodiments, a pharmaceutically acceptable salt, or generally a salt, of such a compound comprises two or more cations, which can be the same or different. In some embodiments, in a pharmaceutically acceptable salt (or generally, a salt), each acidic group having sufficient acidity independently exists as its salt form (e.g., in an oligonucleotide comprising natural phosphate linkages and phosphorothioate internucleotidic linkages, each of the natural phosphate linkages and phosphorothioate internucleotidic linkages independently exists as its salt form). In some embodiments, a pharmaceutically acceptable salt of an oligonucleotide, e.g., a provided oligonucleotide, is a sodium salt of an oligonucleotide. In some embodiments, a pharmaceutically acceptable salt of an oligonucleotide, e.g., an oligonucleotide, is a sodium salt of such an oligonucleotide, wherein each acidic linkage, e.g., each natural phosphate linkage and phosphorothioate internucleotidic linkage, exists as a sodium salt form (all sodium salt).

Protecting group: The term “protecting group,” as used herein, is well known in the art and includes those described in detail in Protecting Groups in Organic Synthesis, T. W. Greene and P. G. M. Wuts, 3^{rd} edition. John Wiley & Sons, 1999, the entirety of which is incorporated herein by reference. Also included are those protecting groups specially adapted for nucleoside and nucleotide chemistry, e.g., those described in Current Protocols in Nucleic Acid Chemistry, edited by Serge L. Beaucage et al. 06/2012, the entirety of Chapter 2 is incorporated herein by reference. Suitable amino-protecting groups include methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative. N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, I-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6 -trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-4o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, a-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′ -dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxy methyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-49-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifluoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group.

In some embodiments, a phosphorous protecting group is a group attached to the internucleotide phosphorous linkage throughout oligonucleotide synthesis. In some embodiments, the phosphorous protecting group is attached to the sulfur atom of the internucleotide phosphorothioate linkage. In some embodiments, the phosphorous protecting group is attached to the oxygen atom of the internucleotide phosphorothioate linkage. In some embodiments, the phosphorous protecting group is attached to the oxygen atom of the internucleotide phosphate linkage. In some embodiments the phosphorous protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, 4-[N-methyl-N-(2,2,2-trifluoroacetyl)amino]butyl.

Protein: As used herein, the term “protein” refers to a polypeptide (i.e., a string of at least two amino acids linked to one another by peptide bonds). In some embodiments, proteins include only naturally-occurring amino acids. In some embodiments, proteins include one or more non-naturally-occurring amino acids (e.g., moieties that form one or more peptide bonds with adjacent amino acids). In some embodiments, one or more residues in a protein chain contain a non-amino-acid moiety (e.g., a glycan, etc). In some embodiments, a protein includes more than one polypeptide chain, for example linked by one or more disulfide bonds or associated by other means. In some embodiments, proteins contain L-amino acids, D-amino acids, or both: in some embodiments, proteins contain one or more amino acid modifications or analogs known in the art. Useful modifications include, e.g., terminal acetylation, amidation, methylation, etc. The term “peptide” is generally used to refer to a polypeptide having a length of less than about 100 amino acids, less than about 50 amino acids, less than 20 amino acids, or less than 10 amino acids.

Subject: As used herein, the term “subject” or “test subject” refers to any organism to which a provided compound or composition is administered in accordance with the present disclosure e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition, e.g., muscular dystrophy.

Substantially: As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual who is “suffering from” a disease, disorder, and/or condition, e.g., muscular dystrophy has been diagnosed with and/or displays one or more symptoms of the disease, disorder, and/or condition, e.g., muscular dystrophy.

Susceptible to: An individual who is “susceptible to” a disease, disorder, and/or condition, e.g., muscular dystrophy is one who has a higher risk of developing the disease, disorder, and/or condition than does a member of the general public. In some embodiments, an individual who is susceptible to a disease, disorder, and/or condition, e.g. muscular dystrophy may not have been diagnosed with the disease, disorder, and/or condition. In some embodiments, an individual who is susceptible to a disease, disorder, and/or condition, e.g., muscular dystrophy may exhibit symptoms of the disease, disorder, and/or condition. In some embodiments, an individual who is susceptible to a disease, disorder, and/or condition, e.g., muscular dystrophy may not exhibit symptoms of the disease, disorder, and/or condition. In some embodiments, an individual who is susceptible to a disease, disorder, and/or condition, e.g., muscular dystrophy will develop the disease, disorder, and/or condition. In some embodiments, an individual who is susceptible to a disease, disorder, and/or condition, e.g., muscular dystrophy will not develop the disease, disorder, and/or condition.

Systemic: The phrases “systemic administration,” “administered systemically,” “peripheral administration,” and “administered peripherally” as used herein have their art-understood meaning referring to administration of a compound or composition such that it enters the recipient's system.

Tautomeric forms: The phrase “tautomeric forms,” as used herein and generally understood in the art, is used to describe different isomeric forms of organic compounds that are capable of facile interconversion. Tautomers may be characterized by the formal migration of a hydrogen atom or proton, accompanied by a switch of a single bond and adjacent double bond. In some embodiments, tautomers may result from prototropic tautomerism (i.e., the relocation of a proton). In some embodiments, tautomers may result from valence tautomerism (i.e., the rapid reorganization of bonding electrons). All such tautomeric forms are intended to be included within the scope of the present disclosure. In some embodiments, tautomeric forms of a compound exist in mobile equilibrium with each other, so that attempts to prepare the separate substances results in the formation of a mixture. In some embodiments, tautomeric forms of a compound are separable and isolatable compounds. In some embodiments of the disclosure, chemical compositions may be provided that are or include pure preparations of a single tautomeric form of a compound. In some embodiments of the disclosure, chemical compositions may be provided as mixtures of two or more tautomeric forms of a compound. In certain embodiments, such mixtures contain equal amounts of different tautomeric forms; in certain embodiments, such mixtures contain different amounts of at least two different tautomeric forms of a compound. In some embodiments of the disclosure, chemical compositions may contain all tautomeric forms of a compound. In some embodiments of the disclosure, chemical compositions may contain less than all tautomeric forms of a compound. In some embodiments of the disclosure, chemical compositions may contain one or more tautomeric forms of a compound in amounts that vary over time as a result of interconversion. In some embodiments of the disclosure, the tautomerism is keto-enol tautomerism. One of skill in the chemical arts would recognize that a keto-enol tautomer can be “trapped” (i.e., chemically modified such that it remains in the “enol” form) using any suitable reagent known in the chemical arts in to provide an enol derivative that may subsequently be isolated using one or more suitable techniques known in the art. Unless otherwise indicated, the present disclosure encompasses all tautomeric forms of relevant compounds, whether in pure form or in admixture with one another.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition, e.g., muscular dystrophy.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, e.g., muscular dystrophy, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition, e.g., muscular dystrophy is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are utilized to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition, e.g., muscular dystrophy. Treatment may be administered to a subject who does not exhibit signs of a disease, disorder, and/or condition, e.g., muscular dystrophy. In some embodiments, treatment may be administered to a subject who exhibits only early signs of the disease, disorder, and/or condition, for example for the purpose of decreasing the risk of developing pathology associated with the disease, disorder, and/or condition.

Unit dose: The expression “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Unsaturated: The term “unsaturated.” as used herein, means that a moiety has one or more units of unsaturation.

Wild-type: As used herein, the term “wild-type” has its art-understood meaning that refers to an entity having a structure and/or activity as found in nature in a “normal” (as contrasted with mutant, diseased, altered, etc) state or context. Those of ordinary skill in the art will appreciate that wild type genes and polypeptides often exist in multiple different forms (e.g., alleles).

Nucleic acid: The term “nucleic acid” includes any nucleotides, analogs thereof, and polymers thereof. The term “polynucleotide” as used herein refer to a polymeric form of nucleotides of any length, either ribonucleotides (RNA) or deoxyribonucleotides (DNA) or analogs thereof. These terms refer to the primary structure of the molecules and include double- and single-stranded DNA, and double- and single-stranded RNA. These terms include, as equivalents, analogs of either RNA or DNA made from nucleotide analogs and modified polynucleotides such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified phosphorus-atom bridges (also referred to herein as “internucleotidic linkages”). The term encompasses nucleic acids containing any combinations of nucleobases, modified nucleobases, sugars, modified sugars, natural phosphate internucleotidic linkages or non-natural internucleotidic linkages. Examples include, and are not limited to, nucleic acids containing ribose moieties, nucleic acids containing deoxy-ribose moieties, nucleic acids containing both ribose and deoxyribose moieties, nucleic acids containing ribose and modified ribose moieties. Unless otherwise specified, the prefix poly- refers to a nucleic acid containing 2 to about 10,000 nucleotide monomer units and wherein the prefix oligo- refers to a nucleic acid containing 2 to about 200 nucleotide monomer units.

Nucleotide: The term “nucleotide” as used herein refers to a monomeric unit of a polynucleotide that consists of a heterocyclic base, a sugar, and one or more phosphate groups or phosphorus-containing internucleotidic linkages. Naturally occurring bases, (guanine, (G), adenine. (A), cytosine, (C), thymine, (T), and uracil (U)) are derivatives of purine or pyrimidine, though it should be understood that naturally and non-naturally occurring base analogs are also included. Naturally occurring sugars include the pentose (five-carbon sugar) deoxyribose (which is found in natural DNA) or ribose (which is found in natural RNA), though it should be understood that naturally and non-naturally occurring sugar analogs are also included, such as sugars with 2′-modifications, sugars in locked nucleic acid (LNA) and phosphorodiamidate morpholino oligomer (PMO). Nucleotides are linked via internucleotidic linkages to form nucleic acids, or polynucleotides. Many internucleotidic linkages are known in the art (such as, though not limited to, natural phosphate linkage, phosphorothioate linkages, boranophosphate linkages and the like). Artificial nucleic acids include PNAs (peptide nucleic acids), phosphotriesters, phosphorothionates, H-phosphonates, phosphoramidates, boranophosphates, methylphosphonates, phosphonoacetates, thiophosphonoacetates and other variants of the phosphate backbone of native nucleic acids, etc. In some embodiments, a nucleotide is a natural nucleotide comprising a naturally occurring nucleobase, a natural occurring sugar and the natural phosphate linkage. In some embodiments, a nucleotide is a modified nucleotide or a nucleotide analog, which is a structural analog that can be used in lieu of a natural nucleotide.

Modified nucleotide: The term “modified nucleotide” includes any chemical moiety which differs structurally from a natural nucleotide but is capable of performing at least one function of a natural nucleotide. In some embodiments, a modified nucleotide comprises a modification at a sugar, base and/or internucleotidic linkage. In some embodiments, a modified nucleotide comprises a modified sugar, modified nucleobase and/or modified internucleotidic linkage. In some embodiments, a modified nucleotide is capable of at least one function of a nucleotide, e.g., forming a subunit in a polymer capable of base-pairing to a nucleic acid comprising an at least complementary sequence of bases.

Analog: The term “analog” includes any chemical moiety which differs structurally from a reference chemical moiety or class of moieties, but which is capable of performing at least one function of such a reference chemical moiety or class of moieties. As non-limiting examples, a nucleotide analog differs structurally from a nucleotide but performs at least one function of a nucleotide; a nucleobase analog differs structurally from a nucleobase but performs at least one function of a nucleobase: a sugar analog differs structurally from a nucleobase but performs at least one function of a sugar, etc.

Nucleoside: The term “nucleoside” refers to a moiety wherein a nucleobase or a modified nucleobase is covalently bound to a sugar or modified sugar.

Modified nucleoside: The term “modified nucleoside” refers to a chemical moiety which is chemically distinct from a natural nucleoside, but which is capable of performing at least one function of a nucleoside. In some embodiments, a modified nucleoside is derived from or chemically similar to a natural nucleoside, but which comprises a chemical modification which differentiates it from a natural nucleoside. Non-limiting examples of modified nucleosides include those which comprise a modification at the base and/or the sugar. Non-limiting examples of modified nucleosides include those with a 2′-modification at a sugar. Non-limiting examples of modified nucleosides also include abasic nucleosides (which lack a nucleobase). In some embodiments, a modified nucleoside is capable of at least one function of a nucleoside, e.g., forming a moiety in a polymer capable of base-pairing to a nucleic acid comprising an at least complementary sequence of bases.

Nucleoside analog: The term “nucleoside analog” refers to a chemical moiety which is chemically distinct from a natural nucleoside, but which is capable of performing at least one function of a nucleoside. In some embodiments, a nucleoside analog comprises an analog of a sugar and/or an analog of a nucleobase. In some embodiments, a modified nucleoside is capable of at least one function of a nucleoside, e.g., forming a moiety in a polymer capable of base-pairing to a nucleic acid comprising a complementary sequence of bases.

Sugar: The term “sugar” refers to a monosaccharide or polysaccharide in closed and/or open form. In some embodiments, sugars are monosaccharides. In some embodiments, sugars are polysaccharides. Sugars include, but are not limited to, ribose, deoxyribose, pentofuranose, pentopyranose, and hexopyranose moieties. As used herein, the term “sugar” also encompasses structural analogs used in lieu of conventional sugar molecules, such as glycol, polymer of which forms the backbone of the nucleic acid analog, glycol nucleic acid (“GNA”), etc. As used herein, the term “sugar” also encompasses structural analogs used in lieu of natural or naturally-occurring nucleotides, such as modified sugars and nucleotide sugars. In some embodiments, a sugar is D-2-deoxyribose. In some embodiments, a sugar is beta-D-deoxyribofuranose. In some embodiments, a sugar moiety is a beta-D-deoxyribofuranose moiety. In some embodiments, a sugar is D-ribose. In some embodiments, a sugar is beta-D-ribofuranose. In some embodiments, a sugar moiety is a beta-D-ribofuranose moiety. In some embodiments, a sugar is optionally substituted beta-D-deoxyribofuranose or beta-D-ribofuranose. In some embodiments, a sugar moiety is an optionally substituted beta-D-deoxyribofuranose or beta-D-ribofuranose moiety. In some embodiments, a sugar moiety/unit in an oligonucleotide, e.g., a DMD oligonucleotide, nucleic acid, etc. is a sugar which comprises one or more carbon atoms each independently connected to an internucleotidic linkage, e.g., optionally substituted beta-D-deoxyribofuranose or beta-D-ribofuranose whose 5′-C and/or 3′-C are each independently connected to an internucleotidic linkage (e.g., a natural phosphate linkage, a modified internucleotidic linkage, a chirally controlled internucleotidic linkage, etc.).

Modified sugar: The term “modified sugar” refers to a moiety that can replace a sugar. A modified sugar mimics the spatial arrangement, electronic properties, or some other physicochemical property of a sugar. In some embodiments, a modified sugar is substituted beta-D-deoxyribofuranose or beta-D-ribofuranose. In some embodiments, a modified sugar comprises a 2′-modification. In some embodiments, a modified sugar comprises a linker (e.g., optionally substituted bivalent heteroaliphatic) connecting two sugar carbon atoms (e.g., C2 and C4), e.g., as found in LNA. In some embodiments, a linker is —O—CH(R)—, wherein R is as described in the present disclosure. In some embodiments, a linker is —O—CH(R)—, wherein O is connected to C2, and —CH(R)— is connected to C4 of a sugar, and R is as described in the present disclosure. In some embodiments, R is methyl. In some embodiments, R is —H. In some embodiments, —CH(R)— is of S configuration. In some embodiments. —CH(R)— is of R configuration.

Nucleobase: The term “nucleobase” refers to the parts of nucleic acids that are involved in the hydrogen-bonding that binds one nucleic acid strand to another complementary strand in a sequence specific manner. The most common naturally-occurring nucleobases are adenine (A), guanine (G), uracil (U), cytosine (C), and thymine (T). In some embodiments, a modified nucleobase is a substituted nucleobase which nucleobase is selected from A, T, C, G, U, and tautomers thereof. In some embodiments, the naturally-occurring nucleobases are modified adenine, guanine, uracil, cytosine, or thymine. In some embodiments, the naturally-occurring nucleobases are methylated adenine, guanine, uracil, cytosine, or thymine. In some embodiments, a nucleobase is a “modified nucleobase,” e.g., a nucleobase other than adenine (A), guanine (G), uracil (U), cytosine (C), and thymine (T). In some embodiments, the modified nucleobases are methylated adenine, guanine, uracil, cytosine, or thymine. In some embodiments, the modified nucleobase mimics the spatial arrangement, electronic properties, or some other physicochemical property of the nucleobase and retains the property of hydrogen-bonding that binds one nucleic acid strand to another in a sequence specific manner. In some embodiments, a modified nucleobase can pair with all of the five naturally occurring bases (uracil, thymine, adenine, cytosine, or guanine) without substantially affecting the melting behavior, recognition by intracellular enzymes or activity of the oligonucleotide duplex. As used herein, the term “nucleobase” also encompasses structural analogs used in lieu of natural or naturally-occurring nucleotides, such as modified nucleobases and nucleobase analogs. In some embodiments, a nucleobase is an optionally substituted A. T, C, G, or U, or a substituted nucleobase which nucleobase is selected from A, T, C, G, U, and tautomers thereof.

Modified nucleobase: The terms “modified nucleobase”, “modified base” and the like refer to a chemical moiety which is chemically distinct from a nucleobase, but which is capable of performing at least one function of a nucleobase. In some embodiments, a modified nucleobase is a nucleobase which comprises a modification. In some embodiments, a modified nucleobase is capable of at least one function of a nucleobase, e.g., forming a moiety in a polymer capable of base-pairing to a nucleic acid comprising an at least complementary sequence of bases. In some embodiments, a modified nucleobase is a substituted nucleobase which nucleobase is selected from A, T, C, G, U, and tautomers thereof.

Chiral ligand: The term “chiral ligand” or “chiral auxiliary” refers to a moiety that is chiral and can be incorporated into a reaction so that the reaction can be carried out with certain stereoselectivity. In some embodiments, the term may also refer to a compound that comprises such a moiety.

Blocking group: The term “blocking group” refers to a group that masks the reactivity of a functional group. The functional group can be subsequently unmasked by removal of the blocking group. In some embodiments, a blocking group is a protecting group.

Moiety: The term “moiety” refers to a specific segment or functional group of a molecule. Chemical moieties are often recognized chemical entities embedded in or appended to a molecule. In some embodiments, a moiety of a compound is a monovalent, bivalent, or polyvalent group formed from the compound by removing one or more —H and/or equivalents thereof from a compound. In some embodiments, depending on its context, “moiety” may also refer to a compound or entity from which the moiety is derived from.

Reading frame: The term “reading frame” refers to one of the six possible reading frames, three in each direction, of a double stranded DNA molecule. The reading frame that is used determines which codons are used to encode amino acids within the coding sequence of a DNA molecule.

Oligonucleotide: the term “oligonucleotide” refers to a polymer or oligomer of nucleotide monomers, containing any combination of nucleobases, modified nucleobases, sugars, modified sugars, natural phosphate linkages, or non-natural internucleotidic linkages.

Oligonucleosides of the present disclosure can be of various lengths. In particular embodiments, oligonucleosides can range from about 20 to about 200 nucleosides in length. In various related embodiments, oligonucleosides, single-stranded, double-stranded, and triple-stranded, can range in length from about 4 to about 10 nucleosides, from about 10 to about 50 nucleosides, from about 20 to about 50 nucleosides, from about 15 to about 30 nucleosides, from about 20 to about 30 nucleosides in length. In some embodiments, the oligonucleoside is from about 9 to about 39 nucleosides in length. In some embodiments, the oligonucleoside is at least 15 nucleosides in length. In some embodiments, the oligonucleoside is at least 20 nucleosides in length. In some embodiments, the oligonucleoside is at least 25 nucleosides in length. In some embodiments, the oligonucleoside is at least 30 nucleosides in length. In some embodiments, the oligonucleoside is a duplex of complementary strands of at least 18 nucleosides in length. In some embodiments, the oligonucleoside is a duplex of complementary strands of at least 21 nucleosides in length. In some embodiments, for the purpose of oligonucleotide lengths, each nucleoside counted independently comprises an optionally substituted nucleobase selected from A, T, C, G, U and their tautomers.

Internucleotidic linkage: As used herein, the phrase “internucleotidic linkage” refers generally to a linkage, typically a phosphorus-containing linkage, between nucleotide units of a nucleic acid or an oligonucleotide, and is interchangeable with “inter-sugar linkage”, “internucleosidic linkage,” and “phosphorus atom bridge,” as used above and herein. As appreciated by those skilled in the art, natural DNA and RNA contain natural phosphate linkages. In some embodiments, an internucleotidic linkage is a natural phosphate linkage (—OP(O)(OH)O—, typically existing as its anionic form —OP(O)(O⁻)O— at pH e.g., ~7.4), as found in naturally occurring DNA and RNA molecules. In some embodiments, an internucleotidic linkage is a modified internucleotidic linkage (or non-natural internucleotidic linkage), which is structurally different from a natural phosphate linkage but may be utilized in place of a natural phosphate linkage, e.g., phosphorothioate internucleotidic linkage, PMO linkages, etc. In some embodiments, an internucleotidic linkage is a modified internucleotidic linkage wherein one or more oxygen atoms of a natural phosphodiester linkage are independently replaced by one or more organic or inorganic moieties. In some embodiments, such an organic or inorganic moiety is selected from but not limited to ═S, ═Se, ═NR′, —SR′, —SeR′, —N(R′)₂, B(R′)₃—S—, —Se—, and —N(R′)—, wherein each R′ is independently as defined and described below. In some embodiments, an internucleotidic linkage is a phosphotriester linkage. In some embodiments, an internucleotidic linkage is a phosphorothioate diester linkage (phosphorothioate internucleotidic linkage,

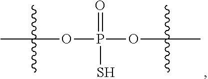 [SMILES: *OP(=O)(S)O* ; ","]

typically existing as its anionic form —OP(O)(S⁻)O— at pH e.g., ~7.4). It is understood by a person of ordinary skill in the art that an internucleotidic linkage may exist as an anion or cation at a given pH due to the existence of acid or base moieties in the linkage.

Unless otherwise specified, the Rp/Sp designations preceding an oligonucleotide sequence describe the configurations of linkage phosphorus in chirally controlled internucleotidic linkages sequentially from 5′ to 3′ of the oligonucleotide sequence.

Oligonucleotide type: As used herein, the phrase “oligonucleotide type” is used to define oligonucleotides that have a particular base sequence, pattern of backbone linkages (i.e., pattern of internucleotidic linkage types, for example, natural phosphate linkages, phosphorothioate internucleotidic linkages, negatively charged internucleotidic linkages, neutral internucleotidic linkages etc), pattern of backbone chiral centers (i.e. pattern of linkage phosphorus stereochemistry (Rp/Sp)), and pattern of backbone phosphorus modifications. In some embodiments, oligonucleotides of a common designated “type” are structurally identical to one another.

One of skill in the art will appreciate that synthetic methods of the present disclosure provide for a degree of control during the synthesis of an oligonucleotide (e.g., a DMD oligonucleotide) strand such that each nucleotide unit of the oligonucleotide strand can be designed and/or selected in advance to have a particular stereochemistry at the linkage phosphorus and/or a particular modification at the linkage phosphorus, and/or a particular base, and/or a particular sugar. In some embodiments, an oligonucleotide strand is designed and/or selected in advance to have a particular combination of stereocenters at the linkage phosphorus. In some embodiments, an oligonucleotide strand is designed and/or determined to have a particular combination of modifications at the linkage phosphorus. In some embodiments, an oligonucleotide strand is designed and/or selected to have a particular combination of bases. In some embodiments, an oligonucleotide strand is designed and/or selected to have a particular combination of one or more of the above structural characteristics. The present disclosure provides compositions comprising or consisting of a plurality of oligonucleotide molecules (e.g., chirally controlled oligonucleotide compositions). In some embodiments, all such molecules are of the same type. In some embodiments, all such molecules are structurally identical to one another. In some embodiments, provided compositions comprise a plurality of oligonucleotides of different types, typically in pre-determined (non-random) relative amounts. In some embodiments, an oligonucleotide is a DMD oligonucleotide as described herein.

Chiral control: As used herein. “chiral control” refers to control of the stereochemical designation of a chiral linkage phosphorus in a chiral internucleotidic linkage within an oligonucleotide (e.g., a DMD oligonucleotide). In some embodiments, a control is achieved through a chiral element that is absent from the sugar and base moieties of an oligonucleotide, for example, in some embodiments, a control is achieved through use of one or more chiral auxiliaries during oligonucleotide preparation as exemplified in the present disclosure, which chiral auxiliaries often are part of chiral phosphoramidites used during oligonucleotide preparation. In contrast to chiral control, a person having ordinary skill in the art appreciates that conventional oligonucleotide synthesis which does not use chiral auxiliaries cannot control stereochemistry at a chiral internucleotidic linkage if such conventional oligonucleotide synthesis is used to form the chiral internucleotidic linkage. In some embodiments, the stereochemical designation of each chiral linkage phosphorus in a chiral internucleotidic linkage within an oligonucleotide is controlled.

Chirally controlled oligonucleotide composition: The terms “chirally controlled (stereocontrolled or stereodefined) oligonucleotide composition”, “chirally controlled (stereocontrolled or stereodefined) nucleic acid composition”, and the like, as used herein, refers to a composition that comprises a plurality of oligonucleotides (or nucleic acids, chirally controlled oligonucleotides or chirally controlled nucleic acids) which share 1) a common base sequence, 2) a common pattern of backbone linkages; 3) a common pattern of backbone chiral centers, and 4) a common pattern of backbone phosphorus modifications (oligonucleotides of a particular type), wherein the plurality of oligonucleotides (or nucleic acids) share the same stereochemistry at one or more chiral internucleotidic linkages (chirally controlled internucleotidic linkages, whose chiral linkage phosphorus is Rp or Sp, not a random Rp and Sp mixture as non-chirally controlled internucleotidic linkages). Level of the plurality of oligonucleotides (or nucleic acids) in a chirally controlled oligonucleotide composition is non-random (pre-determined, controlled). Chirally controlled oligonucleotide compositions are typically prepared through chirally controlled oligonucleotide preparation to stereoselectively form one or more chiral internucleotidic linkages (e.g., using chiral auxiliaries as exemplified in the present disclosure, compared to non-chirally controlled (stereorandom, non-stereoselective, racemic) oligonucleotide synthesis such as traditional phosphoramidite-based oligonucleotide synthesis using no chiral auxiliaries or chiral catalysts to purposefully control stereoselectivity). A chirally controlled oligonucleotide composition is enriched, relative to a substantially racemic preparation of oligonucleotides having the common base sequence, the common pattern of backbone linkages, and the common pattern of backbone phosphorus modifications, for oligonucleotides of the plurality. In some embodiments, a chirally controlled oligonucleotide composition comprises a plurality of oligonucleotides of a particular oligonucleotide type defined by: 1) base sequence; 2) pattern of backbone linkages, 3) pattern of backbone chiral centers, and 4) pattern of backbone phosphorus modifications, wherein it is enriched, relative to a substantially racemic preparation of oligonucleotides having the same base sequence, pattern of backbone linkages, and pattern of backbone phosphorus modifications, for oligonucleotides of the particular oligonucleotide type. As one having ordinary skill in the art readily appreciates, such enrichment can be characterized in that compared to a substantially racemic preparation, at each chirally controlled internucleotidic linkage, a higher level of the linkage phosphorus has the desired configuration. In some embodiments, each chirally controlled internucleotidic linkage independently has a diastereopurity of at least 80%, 85%, 90%, 91%, 92%, 93%, 94%, 95%, 96%, 97%, 98%, or 99% with respect to its chiral linkage phosphorus. In some embodiments, each independently has a diastereopurity of at least 90%. In some embodiments, each independently has a diastereopurity of at least 95%. In some embodiments, each independently has a diastereopurity of at least 97%. In some embodiments, each independently has a diastereopurity of at least 98%. In some embodiments, oligonucleotides of a plurality have the same constitution. In some embodiments, oligonucleotides of a plurality have the same constitution and stereochemistry, and are structurally identical.

In some embodiments, the plurality of oligonucleotides in a chirally controlled oligonucleotide composition share the same base sequence, the same, if any, nucleobase, sugar, and internucleotidic linkage modifications, and the same stereochemistry (Rp or Sp) independently at linkage phosphorus chiral centers of one or more chirally controlled internucleotidic linkages, though stereochemistry of certain linkage phosphorus chiral centers may differ. In some embodiments, about 0.1%-100%, (e.g., about 1%-100%, 5%-100%, 10%-100%, 20%-100%, 30%-100%, 40%-100%, 50%-100%, 60%-100%, 70%-100%, 80%-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition are oligonucleotides of the plurality. In some embodiments, about 0.1%-100%, (e.g., about 1%-100%, 5%-100%, 10%-100%, 20%-100%, 30%-100%, 40%-100%, 50%/9-100%, 60%-100%, 700%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence are oligonucleotides of the plurality. In some embodiments, about 0.1%-100%, (e.g., about 1%-100%, 5%-100%, 10%-100%, 20%-100%, 30%/6-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence, the common pattern of backbone linkages, and the common pattern of backbone phosphorus modifications are oligonucleotides of the plurality. In some embodiments, about 0.1%-100%, (e.g., about 1%-100%, 50%-100%, 0%-100%, 20%-100%, 30%-100%, 40%-100%, 50%-100%, 60%-100%, 70%-100%, 80-100%, 90-100%, 95%-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition, or of all oligonucleotides in a composition that share a common base sequence (e.g., of a plurality of oligonucleotide or an oligonucleotide type), or of all oligonucleotides in a composition that share a common base sequence, a common pattern of backbone linkages, and a common pattern of backbone phosphorus modifications (e.g., of a plurality of oligonucleotide or an oligonucleotide type), or of all oligonucleotides in a composition that share a common base sequence, a common patter of base modifications, a common pattern of sugar modifications, a common pattern of internucleotidic linkage types, and/or a common pattern of internucleotidic linkage modifications (e.g., of a plurality of oligonucleotide or an oligonucleotide type), or of all oligonucleotides in a composition that share the same constitution, are oligonucleotides of the plurality. In some embodiments, a percentage is at least (DP)NCI, wherein DP is a percentage selected from 85%-100%, and NCI is the number of chirally controlled internucleotidic linkage. In some embodiments. DP is at least 85%, 90%, 91%, 92%, 93%, 94%, 95%, 96%, 97%, 98%, or 99%. In some embodiments, DP is at least 85%. In some embodiments, DP is at least 90%. In some embodiments, DP is at least 95%. In some embodiments, DP is at least 96%. In some embodiments, DP is at least 97%. In some embodiments, DP is at least 98%. In some embodiments, DP is at least 99%. In some embodiments, DP reflects diastereopurity of linkage phosphorus chiral centers chirally controlled internucleotidic linkages. In some embodiments, diastereopurity of a linkage phosphorus chiral center of an internucleotidic linkage may be typically assessed using an appropriate dimer comprising such an internucleotidic linkage and the two nucleoside units being linked by the internucleotidic linkage. In some embodiments, the plurality of oligonucleotides share the same stereochemistry at about 1-50 (e.g., about 1-10, 1-20, 5-10, 5-20, 10-15, 10-20, 10-25, 10-30, or about 1, 2, 3, 4, 5, 6, 7, 8, 9, 10, 11, 12, 13, 14, 15, 16, 17, 18, 19, or 20, or at least 1, 2, 3, 4, 5, 6, 7, 8, 9, 10, 11, 12, 13, 14, 15, 16, 17, 18, 19, or 20) chiral internucleotidic linkages. In some embodiments, the plurality of oligonucleotides share the same stereochemistry at about 0.1%-100% (e.g., about 1%-100%, 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 99%) of chiral internucleotidic linkages. In some embodiments, each chiral internucleotidic linkage is a chiral controlled internucleotidic linkage, and the composition is a completely chirally controlled oligonucleotide composition. In some embodiments, not all chiral internucleotidic linkages are chiral controlled internucleotidic linkages, and the composition is a partially chirally controlled oligonucleotide composition. In some embodiments, a chirally controlled oligonucleotide composition comprises predetermined levels of individual oligonucleotide or nucleic acids types. For instance, in some embodiments a chirally controlled oligonucleotide composition comprises one oligonucleotide type at a predetermined level (e.g., as described above). In some embodiments, a chirally controlled oligonucleotide composition comprises more than one oligonucleotide type, each independently at a predetermined level. In some embodiments, a chirally controlled oligonucleotide composition comprises multiple oligonucleotide types, each independently at a predetermined level. In some embodiments, a chirally controlled oligonucleotide composition is a composition of oligonucleotides of an oligonucleotide type, which composition comprises a predetermined level of a plurality of oligonucleotides of the oligonucleotide type. In some embodiments, a chirally controlled oligonucleotide composition is a chirally controlled DMD oligonucleotide composition comprising a plurality of DMD oligonucleotides. In some embodiments, a chirally controlled oligonucleotide composition is a composition of oligonucleotides of a DMD oligonucleotide type.

Chirally pure: as used herein, the phrase “chirally pure” is used to describe an oligonucleotide or compositions thereof, in which all or nearly all (the rest are impurities) of the oligonucleotide molecules exist in a single diastereomeric form with respect to the linkage phosphorus atoms. In many embodiments, as appreciated by those skilled in the art, a chirally pure oligonucleotide composition is substantially pure in that substantially all of the oligonucleotides in the composition are structurally identical (being the same stereoisomer).

Linkage phosphorus: as defined herein, the phrase “linkage phosphorus” is used to indicate that the particular phosphorus atom being referred to is the phosphorus atom present in an internucleotidic linkage, which phosphorus atom corresponds to the phosphorus atom of a natural phosphate linkage as occurs in naturally occurring DNA and RNA. In some embodiments, a linkage phosphorus atom is in a modified internucleotidic linkage. In some embodiments, a linkage phosphorus atom is chiral.

Internucleotidic linkage: As used herein, the phrase “internucleotidic linkage” refers generally to a linkage, typically a phosphorus-containing linkage, between nucleotide units of a nucleic acid or an oligonucleotide, and is interchangeable with “inter-sugar linkage”, “internucleosidic linkage,” and “phosphorus atom bridge,” as used above and herein. As appreciated by those skilled in the art, natural DNA and RNA contain natural phosphate linkages. In some embodiments, an internucleotidic linkage is a natural phosphate linkage (—OP(O)(OH)O—, typically existing as its anionic form —OP(O)(O⁻)O— at pH e.g., ~7.4), as found in naturally occurring DNA and RNA molecules. In some embodiments, an internucleotidic linkage is a modified internucleotidic linkage (or non-natural internucleotidic linkage), which is structurally different from a natural phosphate linkage but may be utilized in place of a natural phosphate linkage, e.g., phosphorothioate internucleotidic linkage, PMO linkages, etc. In some embodiments, an internucleotidic linkage is a modified internucleotidic linkage wherein one or more oxygen atoms of a natural phosphodiester linkage are independently replaced by one or more organic or inorganic moieties. In some embodiments, such an organic or inorganic moiety is selected from but not limited to ═S, ═Se, ═NR′, —SR′, —SeR′, —N(R′)₂, B(R′)₃, —S—, —Se—, and —N(R′)—, wherein each R′ is independently as defined and described below. In some embodiments, an internucleotidic linkage is a phosphotriester linkage. In some embodiments, an internucleotidic linkage is a phosphorothioate diester linkage (phosphorothioate internucleotidic linkage,

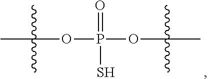 [SMILES: *OP(=O)(S)O* ; ","]

typically existing as its anionic form —OP(O)(S⁻)O— at pH e.g., ~7.4). It is understood by a person of ordinary skill in the art that an internucleotidic linkage may exist as an anion or cation at a given pH due to the existence of acid or base moieties in the linkage. In some embodiments, an internucleotidic linkage is a non-negatively charged internucleotidic linkage at a given pH. In some embodiments, an internucleotidic linkage is a neutral internucleotidic linkage at a given pH. In some embodiments, a given pH is pH ~7.4. In some embodiments, a given pH is in the range of pH about 0, 1, 2, 3, 4, 5, 6 or 7 to pH about 7, 8, 9, 10, 11, 12, 13 or 14. In some embodiments, a given pH is in the range of pH 5-9. In some embodiments, a given pH is in the range of pH 6-8. In some embodiments, an internucleotidic linkage is one of, e.g., PNA (peptide nucleic acid) or PMO (phosphorodiamidate Morpholino oligomer) linkage. In some embodiments, an internucleotidic linkage comprises a chiral linkage phosphorus. In some embodiments, an internucleotidic linkage is a chirally controlled internucleotidic linkage. In some embodiments, an internucleotidic linkage is selected from: s (phosphorothioate), s1, s2, s3, s4, s5, s6, s7, s8, s9, s10, s11, s12, s13, s14, s15, s16, s17 or s18, wherein each of s1, s2, s3, s4, s5, s6, s7, s8, s9, s10, s11, s12, s13, s14, s15, s16, s17 and s18 is independently as described in WO 2017/062862.

Unless otherwise specified, salts, such as pharmaceutically acceptable acid or base addition salts, stereoisomeric forms, and tautomeric forms, of compounds (e.g., DMD oligonucleotides, agents, etc.) are included. Unless otherwise specified, singular forms “a”, “an”, and “the” include the plural reference unless the context clearly indicates otherwise (and vice versa). Thus, for example, a reference to “a compound” may include a plurality of such compounds.

### BRIEF DESCRIPTION OF THE DRAWING

FIG. 1. An example of a HELISA assay.

### DETAILED DESCRIPTION OF CERTAIN EMBODIMENTS

Synthetic oligonucleotides provide useful molecular tools in a wide variety of applications. For example, oligonucleotides are useful in therapeutic, diagnostic, research, and new nanomaterials applications. The use of naturally occurring nucleic acids (e.g., unmodified DNA or RNA) is limited, for example, by their susceptibility to endo- and exo-nucleases. As such, various synthetic counterparts have been developed to circumvent these shortcomings. These include synthetic oligonucleotides that contain chemical modification, e.g., base modifications, sugar modifications, backbone modifications, etc., which, among other things, render these molecules less susceptible to degradation and improve other properties of oligonucleotides, e.g., DMD oligonucleotides. Chemical modifications may also lead to certain undesired effects, such as increased toxicities, etc. From a structural point of view, modifications to natural phosphate linkages can introduce chirality, and certain properties of oligonucleotides may be affected by the configurations of the phosphorus atoms that form the backbone of the oligonucleotides.

In some embodiments, the present disclosure pertains to a DMD oligonucleotide or DMD oligonucleotide composition, which has a sequence at least partially complementary to a DMD target nucleic acid, and, in some embodiments, is capable of mediating skipping of a DMD exon. In some embodiments, a DMD oligonucleotide or DMD oligonucleotide composition is capable of mediating skipping of DMD exon 51.

In some embodiments, a DMD oligonucleotide or DMD oligonucleotide composition comprises any of various modifications to the internucleotidic linkages (e.g., backbone), sugars, and/or nucleobases.

In some embodiments, a DMD oligonucleotide or DMD oligonucleotide composition is any DMD oligonucleotide or DMD oligonucleotide composition disclosed herein (e.g., in Table A1).

In some embodiments, the chirality of the backbone (e.g., the configurations of the phosphorus atoms) or inclusion of natural phosphate linkages or non-natural internucleotidic linkages in the backbone and/or modifications of a sugar and/or nucleobase, and/or the addition of chemical moieties can affect properties and activities of DMD oligonucleotides, e.g., the ability of a DMD oligonucleotide (e.g., a DMD oligonucleotide antisense to a Dystrophin (DMD) DMD transcript sequence) to skip DMD exon 51, and/or other properties of a DMD oligonucleotide, including but not limited to, increased stability, improved pharmacokinetics, and/or decreased immunogenicity, etc. Suitable assays for assessing properties and/or activities of provided compounds, e.g., DMD oligonucleotides, and compositions thereof are widely known in the art and can be utilized in accordance with the present disclosure.

In some embodiments, a DMD transcript is pre-mRNA. In some embodiments, a splicing product is mature RNA. In some embodiments, a splicing product is mRNA. In some embodiments, splicing modulation or alteration comprises skipping DMD exon 51.

In some embodiments, provided DMD oligonucleotides in provided compositions, e.g., DMD oligonucleotides of a plurality, comprise base modifications, sugar modifications, and/or internucleotidic linkage modifications. In some embodiments, provided DMD oligonucleotides comprise base modifications and sugar modifications. In some embodiments, provided DMD oligonucleotides comprise base modifications and internucleotidic linkage modifications. In some embodiments, provided DMD oligonucleotides comprise sugar modifications and internucleotidic modifications. In some embodiments, provided compositions comprise base modifications, sugar modifications, and internucleotidic linkage modifications. Example chemical modifications, such as base modifications, sugar modifications, internucleotidic linkage modifications, etc. are widely known in the art including but not limited to those described in this disclosure. In some embodiments, a modified base is substituted A, T, C, G or U. In some embodiments, a sugar modification is 2′-modification. In some embodiments, a 2′-modification is 2-F modification. In some embodiments, a 2′-modification is 2′-OR¹, wherein R¹ is not hydrogen. In some embodiments, a 2′-modification is 2′-OR¹, wherein R¹ is optionally substituted alkyl. In some embodiments, a 2′-modification is 2′-OMe. In some embodiments, a 2′-modification is 2′-MOE. In some embodiments, a modified sugar moiety is a bridged bicyclic or polycyclic ring. In some embodiments, a modified sugar moiety is a bridged bicyclic or polycyclic ring having 5-20 ring atoms wherein one or more ring atoms are optionally and independently heteroatoms. Example ring structures are widely known in the art, such as those found in BNA, LNA, etc.

In some embodiments, provided DMD oligonucleotides comprise one or more modified internucleotidic linkages. In some embodiments, provided DMD oligonucleotides comprise one or more chiral modified internucleotidic linkages. In some embodiments, provided DMD oligonucleotides comprise one or more chirally controlled chiral modified internucleotidic linkages. In some embodiments, provided DMD oligonucleotides comprise one or more natural phosphate linkages. In some embodiments, provided DMD oligonucleotides comprise one or more modified internucleotidic linkages and one or more natural phosphate linkages. In some embodiments, a modified internucleotidic linkage is a phosphorothioate linkage. In some embodiments, each modified internucleotidic linkage is a phosphorothioate linkage.

In some embodiments, provided DMD oligonucleotides comprise both one or more modified internucleotidic linkages and one or more natural phosphate linkages. In some embodiments, DMD oligonucleotides comprising both modified internucleotidic linkage and natural phosphate linkage and compositions thereof provide improved properties, e.g., skipping of exon 51 and toxicities, etc. In some embodiments, a modified internucleotidic linkage is a chiral internucleotidic linkage. In some embodiments, a modified internucleotidic linkage is a phosphorothioate linkage. In some embodiments, a modified internucleotidic linkage is a substituted phosphorothioate linkage.

Among other things, the present disclosure encompasses the recognition that stereorandom DMD oligonucleotide preparations contain a plurality of distinct chemical entities that differ from one another, e.g., in the stereochemical structure of individual backbone linkage phosphorus chiral centers within the DMD oligonucleotide chain. Without control of stereochemistry of backbone chiral centers, stereorandom DMD oligonucleotide preparations provide uncontrolled compositions comprising undetermined levels of DMD oligonucleotide stereoisomers with respect to the uncontrolled chiral centers, e.g., chiral linkage phosphorus. Even though these stereoisomers may have the same base sequence, they are different chemical entities at least due to their different backbone stereochemistry, and they can have, as demonstrated herein, different properties, e.g., skipping of exon 51, toxicities, etc. Among other things, the present disclosure provides new DMD oligonucleotide compositions wherein stereochemistry of one or more linkage phosphorus chiral centers are independently controlled (e.g., in chirally controlled internucleotidic linkages). In some embodiments, the present disclosure provides chirally controlled DMD oligonucleotide compositions which are or contain particular stereoisomers of DMD oligonucleotides of interest.

In some embodiments, in a DMD oligonucleotide, a pattern of backbone chiral centers can provide improved activity(s) or characteristic(s), including but not limited to: improved skipping of DMD exon 51, increased stability, increased activity, low toxicity, low immune response, improved protein binding profile, increased binding to certain proteins, and/or enhanced delivery.

In some embodiments, provided DMD oligonucleotides comprise one or more non-negatively charged internucleotidic linkages. In some embodiments, a non-negatively charged internucleotidic linkage is a positively charged internucleotidic linkage. In some embodiments, a non-negatively charged internucleotidic linkage is a neutral internucleotidic linkage. In some embodiments, a modified internucleotidic linkage (e.g., a non-negatively charged internucleotidic linkage) comprises an optionally substituted guanidine moiety. In some embodiments, a modified internucleotidic linkage comprises an optionally substituted cyclic guanidine moiety. In some embodiments, a modified internucleotidic linkage comprises an optionally substituted cyclic guanidine moiety and has the structure

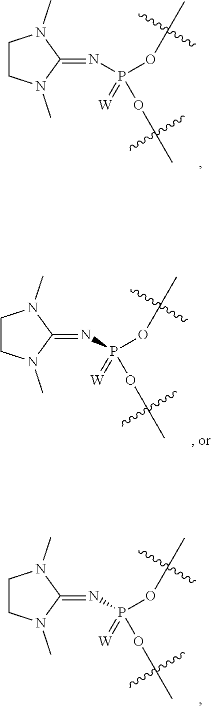

wherein W is O. In some embodiments, a non-negatively charged internucleotidic linkage (e.g., a neutral internucleotidic linkage) has the structure of

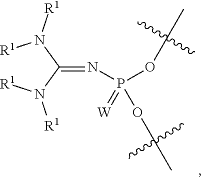

wherein each variable is independently as described herein. In some embodiments, two R¹ (either on the same or different nitrogen atoms) are R and are taken together with their intervening atoms to form an optionally substituted ring as described herein. In some embodiments, a non-negatively charged internucleotidic linkage (e.g., a neutral internucleotidic linkage) has the structure of

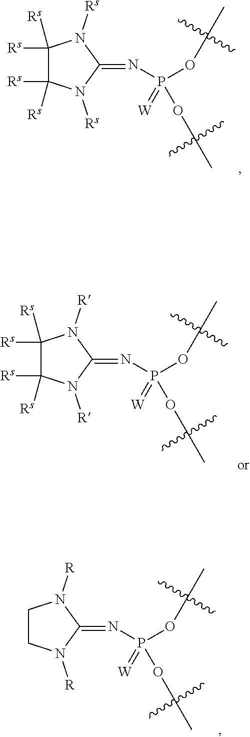

wherein each variable is independently as described herein. In some embodiments, W is O. In some embodiments, such an internucleotidic linkage is chirally controlled. Useful embodiments of various variables, e.g., R¹, R′, R^{s}, etc., include those described in 62/776,432, WO 2019/200185, and WO 2019/217784, description including embodiments of each variable is independently incorporated herein by reference.

In some embodiments, a non-negatively charged internucleotidic linkage is stereochemically controlled.

In some embodiments, provided DMD oligonucleotides can bind to a DMD transcript, and change the splicing pattern of the DMD transcript by inducing (e.g., mediating) skipping of exon 51. In some embodiments, provided DMD oligonucleotides provides exon-skipping of an exon, with efficiency greater than a comparable DMD oligonucleotide under one or more suitable conditions, e.g., as described herein. In some embodiments, a provided skipping efficiency is at least 10%, 20%, 30%, 40%, 50%, 60%, 70%, 80%, 90%, 100%, 110%, 120%, 130%, 140%, 150%, 160%, 170%, 180%, 190% more than, or 2, 3, 4, 5, 6, 7, 8, 9, 10, 11, 12, 13, 14, 15, 16, 17, 18, 19, 20, 30, 40, 50 or more fold of, that of a comparable DMD oligonucleotide under one or more suitable conditions, e.g., as described herein.

In some embodiments, compared to a reference condition, provided chirally controlled DMD oligonucleotide compositions are surprisingly effective. In some embodiments, a change is measured by increase of a desired mRNA level compared to a reference condition. In some embodiments, a change is measured by decrease of an undesired mRNA level compared to a reference condition. In some embodiments, a reference condition is absence of DMD oligonucleotide treatment. In some embodiments, a reference condition is a stereorandom composition of DMD oligonucleotides having the same base sequence and chemical modifications.

In some embodiments, a provided DMD oligonucleotide composition is characterized in that, when it is contacted with the DMD transcript in a DMD transcript splicing system, splicing of the DMD transcript is altered (e.g., exon 51 is skipped) relative to that observed under reference conditions selected from the group consisting of absence of the composition, presence of a reference composition, and combinations thereof. In some embodiments, a desired splicing product (e.g., one lacking exon 51) is increased 10%, 20%, 30%, 40%, 50%, 60%, 70%, 80%, 90%, 100%, or 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, 1000 fold or more. In some embodiments, a desired splicing reference is absent (e.g., cannot be reliably detected by quantitative PCR) under reference conditions. In some embodiments, as exemplified in the present disclosure, levels of the plurality of DMD oligonucleotides, e.g., a plurality of DMD oligonucleotides, in provided compositions are pre-determined.

In some embodiments, DMD oligonucleotides having a common base sequence may have the same pattern of nucleoside modifications. e.g., sugar modifications, base modifications, etc. In some embodiments, a pattern of nucleoside modifications may be represented by a combination of locations and modifications. In some embodiments, all non-chiral linkages (e.g., PO) may be omitted. In some embodiments, DMD oligonucleotides having the same base sequence have the same constitution.

In some embodiments, a DMD oligonucleotide composition is chirally controlled.

In some embodiments, a DMD oligonucleotide composition is not stereorandom, and is not a racemic preparation of a diastereoisomers.

As understood by a person having ordinary skill in the art, a stereorandom or racemic preparation of DMD oligonucleotides is prepared by non-stereoselective and/or low-stereoselective coupling of nucleotide monomers, typically without using any chiral auxiliaries, chiral modification reagents, and/or chiral catalysts. In some embodiments, in a substantially racemic (or chirally uncontrolled) preparation of DMD oligonucleotides, all or most coupling steps are not chirally controlled in that the coupling steps are not specifically conducted to provide enhanced stereoselectivity. An example substantially racemic preparation of DMD oligonucleotides is the preparation of phosphorothioate DMD oligonucleotides through sulfurizing phosphite triesters from commonly used phosphoramidite DMD oligonucleotide synthesis with either tetraethylthiuram disulfide or (TETD) or 3H-1, 2-bensodithiol-3-one 1, 1-dioxide (BDTD), a well-known process in the art. In some embodiments, substantially racemic preparation of DMD oligonucleotides provides substantially racemic DMD oligonucleotide compositions (or chirally uncontrolled DMD oligonucleotide compositions). In some embodiments, at least one coupling of a nucleotide monomer has a diastereoselectivity lower than about 60:40, 70:30, 80:20, 85:15, 90:10, 91:9, 92:8, 97:3, 98:2, or 99:1. In some embodiments, each internucleotidic linkage independently has a diastereoselectivity lower than about 60:40, 70:30, 80:20, 85:15, 90:10, 91:9, 92:8, 97:3, 98:2, or 99:1. In some embodiments, a diastereoselectivity is lower than about 60:40. In some embodiments, a diastereoselectivity is lower than about 70:30. In some embodiments, a diastereoselectivity is lower than about 80:20. In some embodiments, a diastereoselectivity is lower than about 90:10. In some embodiments, a diastereoselectivity is lower than about 91:9. In some embodiments, at least one internucleotidic linkage has a diastereoselectivity lower than about 90:10. In some embodiments, each internucleotidic linkage independently has a diastereoselectivity lower than about 90:10. In some embodiments, a non-chirally controlled internucleotidic linkage has a diastereomeric purity no more than 90%, 85%, 80%, 75%, 70%, 65%, 60%, or 55%. In some embodiments, the purity is no more than 90%. In some embodiments, the purity is no more than 85%. In some embodiments, the purity is no more than 80%.

In contrast, in chirally controlled DMD oligonucleotide composition, at least one and typically each chirally controlled internucleotidic linkage, such as those of DMD oligonucleotides of chirally controlled DMD oligonucleotide compositions, independently has a diastereomeric purity of 90%, 91%, 92%, 93%, 94%, 95%, 96%, 97%, 98%, 99% or more with respect to the chiral linkage phosphorus. In some embodiments, a diastereomeric purity is 95% or more. In some embodiments, a diastereomeric purity is 96% or more. In some embodiments, a diastereomeric purity is 97% or more. In some embodiments, a diastereomeric purity is 98% or more. In some embodiments, a diastereomeric purity is 99% or more. Among other things, technologies of the present disclosure routinely provide chirally controlled internucleotidic linkages with high diastereomeric purity.

As appreciated by a person having ordinary skill in the art, diastereoselectivity of a coupling or diastereomeric purity (diastereopurity) of an internucleotidic linkage can be assessed through the diastereoselectivity of a dimer formation/diastereomeric purity of the internucleotidic linkage of a dimer formed under the same or comparable conditions, wherein the dimer has the same 5′- and 3′-nucleosides and internucleotidic linkage.

In some embodiments, the present disclosure provides an oligonucleotide composition comprising a plurality of oligonucleotides, wherein oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”).

In some embodiments, the present disclosure provides an oligonucleotide composition comprising a plurality of oligonucleotides, wherein oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”).
wherein the composition is enriched, relative to a substantially racemic preparation of oligonucleotides sharing the common base sequence, for oligonucleotides of the plurality.

In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all internucleotidic linkages are chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all chiral internucleotidic linkages are chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%400%, 80%-85%, 80% 90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all phosphorothioate internucleotidic linkages are chirally controlled. In some embodiments, a percentage is at least 50%. In some embodiments, a percentage is at least 60%. In some embodiments, a percentage is at least 70%. In some embodiments, a percentage is at least 80%. In some embodiments, a percentage is at least 90%. In some embodiments, a percentage is at least 90%. In some embodiments, each chiral internucleotidic linkage is chirally controlled. In some embodiments, each phosphorothioate internucleotidic linkage is chirally controlled.

In some embodiments, the present disclosure provides a chirally controlled oligonucleotide composition of an oligonucleotide, wherein the composition is enriched, relative to a substantially racemic preparation of the oligonucleotide, for the oligonucleotide and/or pharmaceutically acceptable salt forms thereof.

In some embodiments, at least about 5%-100%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition are oligonucleotide of the plurality. In some embodiments, at least about 5%-100%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition that are of the same base sequence (e.g., a common base sequence) are oligonucleotide of the plurality. In some embodiments, an enrichment relative to a substantially racemic preparation is that at least about 5%-100%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition are oligonucleotide of the plurality. In some embodiments, an enrichment relative to a substantially racemic preparation is that at least about 5%-100%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition that are of the same base sequence (e.g., a common base sequence) are oligonucleotide of the plurality. In some embodiments, the present disclosure provides a composition of an oligonucleotide, wherein at least about 5%-100%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition are each independently the oligonucleotide in one or more of its various forms (e.g., acid, base, various salt forms, etc.). In some embodiments, the present disclosure provides a composition of an oligonucleotide, wherein at least about 5%-100%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition are each independently the oligonucleotide or a pharmaceutically acceptable salt thereof. In some embodiments, the present disclosure provides a composition of an oligonucleotide, wherein at least about 5%-100%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition that are of the same base sequence as the oligonucleotide are each independently the oligonucleotide in one or more of its various forms (e.g., acid, base, various salt forms, etc.). In some embodiments, the present disclosure provides a composition of an oligonucleotide, wherein at least about 5%-100%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition that are of the same base sequence as the oligonucleotide are each independently the oligonucleotide or a pharmaceutically acceptable salt thereof. In some embodiments, the present disclosure provides a composition of an oligonucleotide, wherein at least about 5%-100%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition that are of the same base sequence and the same patterns of nucleobase, sugar and/or internucleotidic linkage modifications (if any) as the oligonucleotide are each independently the oligonucleotide in one or more of its various forms (e.g., acid, base, various salt forms, etc.). In some embodiments, the present disclosure provides a composition of an oligonucleotide, wherein at least about 5%-100%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition that are of the same base sequence and the same patterns of nucleobase, sugar and/or internucleotidic linkage modifications (if any) as the oligonucleotide are each independently the oligonucleotide or a pharmaceutically acceptable salt thereof. In some embodiments, at least about 5%-100%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition that share one or more features as the oligonucleotide (e.g., as described above) are one or more pharmaceutically acceptable salts of the oligonucleotide. In some embodiments, a composition comprises one and no more than one pharmaceutically acceptable salt of the oligonucleotide. In some embodiments, a composition comprises two or more pharmaceutically acceptable salts of the oligonucleotide. In some embodiments, a composition is a liquid composition and an oligonucleotide and/or its one or more salt forms thereof are dissolved. In some embodiments, a percentage is at least 50%. In some embodiments, it is at least 60%. In some embodiments, it is at least 70%. In some embodiments, it is at least 80%. In some embodiments, it is at least 90%. In some embodiments, it is at least 95%. In some embodiments, base sequence of an oligonucleotide is or comprises a sequence in Table A1. In some embodiments, an oligonucleotide comprises one or more natural phosphate linkages, one or more phosphorothioate internucleotidic linkages, and one or more neutral internucleotidic linkages. In some embodiments, an oligonucleotide is an oligonucleotide described in Table A1, wherein each chiral oligonucleotide is independently Rp or Sp.

In some embodiments, the present disclosure provides chirally controlled (and/or stereochemically pure) DMD oligonucleotide compositions comprising a plurality of DMD oligonucleotides defined by having:
1) a common base sequence;
2) a common pattern of backbone linkages; and
3) a common pattern of backbone chiral centers, which composition is a substantially pure preparation of a single DMD oligonucleotide in that at least about 10% of the DMD oligonucleotides in the composition have the common base sequence and length, the common pattern of backbone linkages, and the common pattern of backbone chiral centers, wherein the oligonucleotide is provided herein (e.g., in Table A1).

In some embodiments, the present disclosure provides chirally controlled DMD oligonucleotide composition of a plurality of DMD oligonucleotides, wherein the composition is enriched, relative to a substantially racemic preparation of the same DMD oligonucleotides, for DMD oligonucleotides of a single DMD oligonucleotide type. In some embodiments, the present disclosure provides chirally controlled DMD oligonucleotide composition of a plurality of DMD oligonucleotides wherein the composition is enriched, relative to a substantially racemic preparation of the same DMD oligonucleotides, for DMD oligonucleotides of a single DMD oligonucleotide type defined by:
1) base sequence;
2) pattern of backbone linkages;
3) pattern of backbone chiral centers; and
4) pattern of backbone phosphorus modifications, wherein the oligonucleotide is provided herein (e.g., in Table A1).

In some embodiments, the present disclosure provides a chirally controlled DMD oligonucleotide composition comprising a plurality of DMD oligonucleotides of a particular DMD oligonucleotide type defined by:
1) base sequence;
2) pattern of backbone linkages;
3) pattern of backbone chiral centers; and
4) pattern of backbone phosphorus modifications,
   wherein the composition is enriched, relative to a substantially racemic preparation of DMD oligonucleotides having the same base sequence and length, for DMD oligonucleotides of the particular DMD oligonucleotide type, wherein the oligonucleotide is provided herein (e.g., in Table A1).

In some embodiments, DMD oligonucleotides of a DMD oligonucleotide type have a common pattern of backbone phosphorus modifications and a common pattern of sugar modifications. In some embodiments, DMD oligonucleotides of a DMD oligonucleotide type have a common pattern of backbone phosphorus modifications and a common pattern of base modifications. In some embodiments, DMD oligonucleotides of a DMD oligonucleotide type have a common pattern of backbone phosphorus modifications and a common pattern of nucleoside modifications. In some embodiments, DMD oligonucleotides of a particular type have the same constitution. In some embodiments, DMD oligonucleotides of a DMD oligonucleotide type are identical.

In some embodiments, a chirally controlled DMD oligonucleotide composition is a substantially pure preparation of a DMD oligonucleotide type in that DMD oligonucleotides in the composition that are not of the DMD oligonucleotide type are impurities form the preparation process of said DMD oligonucleotide type, in some case, after certain purification procedures.

In some embodiments, at least about 20%, 30%, 40%, 50%, 60%, 70%, 80%, 90%, or 95% of the DMD oligonucleotides in the composition have a common base sequence, a common pattern of backbone linkages, and a common pattern of backbone chiral centers.

In some embodiments, DMD oligonucleotides having a common base sequence, a common pattern of backbone linkages, and a common pattern of backbone chiral centers have a common pattern of backbone phosphorus modifications. In some embodiments, DMD oligonucleotides having a common base sequence, a common pattern of backbone linkages, and a common pattern of backbone chiral centers have a common pattern of backbone phosphorus modifications and a common pattern of nucleoside modifications. In some embodiments, DMD oligonucleotides having a common base sequence, a common pattern of backbone linkages, and a common pattern of backbone chiral centers have a common pattern of backbone phosphorus modifications and a common pattern of sugar modifications. In some embodiments, DMD oligonucleotides having a common base sequence, a common pattern of backbone linkages, and a common pattern of backbone chiral centers have a common pattern of backbone phosphorus modifications and a common pattern of base modifications. In some embodiments, DMD oligonucleotides having a common base sequence, a common pattern of backbone linkages, and a common pattern of backbone chiral centers are identical.

In some embodiments, purity of a chirally controlled DMD oligonucleotide composition of a DMD oligonucleotide type is expressed as the percentage of DMD oligonucleotides in the composition that are of the DMD oligonucleotide type. In some embodiments, at least about 10% of the DMD oligonucleotides in a chirally controlled DMD oligonucleotide composition are of the DMD oligonucleotide type. In some embodiments, at least about 20% of the DMD oligonucleotides in a chirally controlled DMD oligonucleotide composition are of the DMD oligonucleotide type. In some embodiments, at least about 30% of the DMD oligonucleotides in a chirally controlled DMD oligonucleotide composition are of the DMD oligonucleotide type. In some embodiments, at least about 40% of the DMD oligonucleotides in a chirally controlled DMD oligonucleotide composition are of the DMD oligonucleotide type. In some embodiments, at least about 50% of the DMD oligonucleotides in a chirally controlled DMD oligonucleotide composition are of the DMD oligonucleotide type. In some embodiments, at least about 60% of the DMD oligonucleotides in a chirally controlled DMD oligonucleotide composition are of the DMD oligonucleotide type. In some embodiments, at least about 70% of the DMD oligonucleotides in a chirally controlled DMD oligonucleotide composition are of the DMD oligonucleotide type. In some embodiments, at least about 80% of the DMD oligonucleotides in a chirally controlled DMD oligonucleotide composition are of the DMD oligonucleotide type. In some embodiments, at least about 90% of the DMD oligonucleotides in a chirally controlled DMD oligonucleotide composition are of the DMD oligonucleotide type. In some embodiments, at least about 92% of the DMD oligonucleotides in a chirally controlled DMD oligonucleotide composition are of the DMD oligonucleotide type. In some embodiments, at least about 94% of the DMD oligonucleotides in a chirally controlled DMD oligonucleotide composition are of the DMD oligonucleotide type. In some embodiments, at least about 95% of the DMD oligonucleotides in a chirally controlled DMD oligonucleotide composition are of the DMD oligonucleotide type. In some embodiments, at least about 96% of the DMD oligonucleotides in a chirally controlled DMD oligonucleotide composition are of the same DMD oligonucleotide type. In some embodiments, at least about 97% of the DMD oligonucleotides in a chirally controlled DMD oligonucleotide composition are of the DMD oligonucleotide type. In some embodiments, at least about 98% of the DMD oligonucleotides in a chirally controlled DMD oligonucleotide composition are of the DMD oligonucleotide type. In some embodiments, at least about 99% of the DMD oligonucleotides in a chirally controlled DMD oligonucleotide composition are of the DMD oligonucleotide type.

In some embodiments, purity of a chirally controlled DMD oligonucleotide composition can be controlled by stereoselectivity of each coupling step in its preparation process. In some embodiments, a coupling step has a stereoselectivity (e.g., diastereoselectivity) of 60% (60% of the new internucleotidic linkage formed from the coupling step has the intended stereochemistry). After such a coupling step, the new internucleotidic linkage formed may be referred to have a 60% purity. In some embodiments, each coupling step has a stereoselectivity of at least 60%. In some embodiments, each coupling step has a stereoselectivity of at least 70%. In some embodiments, each coupling step has a stereoselectivity of at least 80%. In some embodiments, each coupling step has a stereoselectivity of at least 85%. In some embodiments, each coupling step has a stereoselectivity of at least 90%. In some embodiments, each coupling step has a stereoselectivity of at least 91%. In some embodiments, each coupling step has a stereoselectivity of at least 92%. In some embodiments, each coupling step has a stereoselectivity of at least 93%. In some embodiments, each coupling step has a stereoselectivity of at least 94%. In some embodiments, each coupling step has a stereoselectivity of at least 95%. In some embodiments, each coupling step has a stereoselectivity of at least 96%. In some embodiments, each coupling step has a stereoselectivity of at least 97%. In some embodiments, each coupling step has a stereoselectivity of at least 98%. In some embodiments, each coupling step has a stereoselectivity of at least 99%. In some embodiments, each coupling step has a stereoselectivity of at least 99.5%. In some embodiments, each coupling step has a stereoselectivity of virtually 100%.

In some embodiments, in provided compositions, at least 0.5%, 1%, 2%, 3%, 4%, 5%, 6%, 7%, 8%, 9%, 10%, 20%, 30%, 40%, 50%, 60%, 70%, 75%, 80%, 81%, 82%, 83%, 84%, 85%, 86%, 87%, 88%, 89%, 90%, 91%, 92%, 93%, 94%, 95%, 96%, 97%, 97% or 99% of DMD oligonucleotides that have the base sequence of a particular DMD oligonucleotide type (defined by 1) base sequence; 2) pattern of backbone linkages; 3) pattern of backbone chiral centers; and 4) pattern of backbone phosphorus modifications) are DMD oligonucleotides of the particular DMD oligonucleotide type. In some embodiments, at least 0.5%, 1%, 2%, 3%, 4%, 5%, 6%, 7%, 8%, 9%, 10%, 20%, 30%, 40%, 50%, 60%, 70%, 75%, 80%, 81%, 82%, 83%, 84%, 85%, 86%, 87%, 88%, 89%, 90%, 91%, 92%, 93%, 94%, 95%, 96%, 97%, 97% or 99% of DMD oligonucleotides that have the base sequence, the pattern of backbone linkages, and the pattern of backbone phosphorus modifications of a particular DMD oligonucleotide type are DMD oligonucleotides of the particular DMD oligonucleotide type.

In some embodiments, a provided DMD oligonucleotide comprises one or more chiral, modified phosphate linkages. In some embodiments, provided chirally controlled (and/or stereochemically pure) preparations are of DMD oligonucleotides that include one or more modified backbone linkages, bases, and/or sugars.

In some embodiments, provided chirally controlled (and/or stereochemically pure) preparations are of a stereochemical purity of greater than about 80%. In some embodiments, provided chirally controlled (and/or stereochemically pure) preparations are of a stereochemical purity of greater than about 85%. In some embodiments, provided chirally controlled (and/or stereochemically pure) preparations are of a stereochemical purity of greater than about 90%. In some embodiments, provided chirally controlled (and/or stereochemically pure) preparations are of a stereochemical purity of greater than about 91%. In some embodiments, provided chirally controlled (and/or stereochemically pure) preparations are of a stereochemical purity of greater than about 92%. In some embodiments, provided chirally controlled (and/or stereochemically pure) preparations are of a stereochemical purity of greater than about 93%. In some embodiments, provided chirally controlled (and/or stereochemically pure) preparations are of a stereochemical purity of greater than about 94%. In some embodiments, provided chirally controlled (and/or stereochemically pure) preparations are of a stereochemical purity of greater than about 95%. In some embodiments, provided chirally controlled (and/or stereochemically pure) preparations are of a stereochemical purity of greater than about 96%. In some embodiments, provided chirally controlled (and/or stereochemically pure) preparations are of a stereochemical purity of greater than about 97%. In some embodiments, provided chirally controlled (and/or stereochemically pure) preparations are of a stereochemical purity of greater than about 98%. In some embodiments, provided chirally controlled (and/or stereochemically pure) preparations are of a stereochemical purity of greater than about 99%.

In some embodiments, one or more is one. In some embodiments, one or more is two. In some embodiments, one or more is three. In some embodiments, one or more is four. In some embodiments, one or more is five. In some embodiments, one or more is six. In some embodiments, one or more is seven. In some embodiments, one or more is eight. In some embodiments, one or more is nine. In some embodiments, one or more is ten. In some embodiments, one or more is at least one. In some embodiments, one or more is at least two. In some embodiments, one or more is at least three. In some embodiments, one or more is at least four. In some embodiments, one or more is at least five. In some embodiments, one or more is at least six. In some embodiments, one or more is at least seven. In some embodiments, one or more is at least eight. In some embodiments, one or more is at least nine. In some embodiments, one or more is at least ten.

In some embodiments, a base sequence, e.g., a common base sequence of a plurality of DMD oligonucleotide, a base sequence of a particular DMD oligonucleotide type, etc., comprises or is a sequence complementary to a gene or DMD transcript (e.g., of Dystrophin or DMD). In some embodiments, a common base sequence comprises or is a sequence 100/complementary to a gene.

In some embodiments, linkage phosphorus of chiral internucleotidic linkages are chirally controlled. In some embodiments, a chiral internucleotidic linkage is phosphorothioate internucleotidic linkage. In some embodiments, each chiral internucleotidic linkage in a DMD oligonucleotide of a provided composition is a phosphorothioate internucleotidic linkage.

As appreciated by those skilled in the art, internucleotidic linkages, natural phosphate linkages, phosphorothioate internucleotidic linkages, etc. may exist in their salt forms depending on pH of their environment. Unless otherwise indicated, such salt forms are included in the present application when such internucleotidic linkages are referred to.

In some embodiments, DMD oligonucleotides of the present disclosure comprise one or more modified sugar moieties. In some embodiments, DMD oligonucleotides of the present disclosure comprise one or more modified base moieties. As known by a person of ordinary skill in the art and described in the disclosure, various modifications can be introduced to sugar and base moieties. For example, in some embodiments, a modification is a modification described in U.S. Pat. No. 9,006,198, WO2014/012081, WO 2015/107425, and WO 2017/062862, the sugar and base modifications of each of which are incorporated herein by reference.

As used herein in the present disclosure, unless otherwise clear from context, (i) the term “a” or “an” may be understood to mean “at least one”; (ii) the term “or” may be understood to mean “and/or”; (iii) the terms “comprising”. “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more: (v) the terms “about” and “approximately” may be understood to permit standard variation as would be understood by those of ordinary skill in the art; and (vi) where ranges are provided, endpoints are included.

Unless otherwise specified, description of oligonucleotides and elements thereof(e.g., base sequence, sugar modifications, internucleotidic linkages, linkage phosphorus stereochemistry, patterns thereof, etc.) is from 5′ to 3′. As those skilled in the art will appreciate, in some embodiments, oligonucleotides may be provided and/or utilized as salt forms, particularly pharmaceutically acceptable salt forms, e.g., sodium salts. As those skilled in the art will also appreciate, in some embodiments, individual oligonucleotides within a composition may be considered to be of the same constitution and/or structure even though, within such composition (e.g., a liquid composition), particular such oligonucleotides might be in different salt form(s) (and may be dissolved and the oligonucleotide chain may exist as an anion form when, e.g., in a liquid composition) at a particular moment in time. For example, those skilled in the art will appreciate that, at a given pH, individual internucleotidic linkages along an oligonucleotide chain may be in an acid (H) form, or in one of a plurality of possible salt forms (e.g., a sodium salt, or a salt of a different cation, depending on which ions might be present in the preparation or composition), and will understand that, so long as their acid forms (e.g., replacing all cations, if any, with H⁺) are of the same constitution and/or structure, such individual oligonucleotides may properly be considered to be of the same constitution and/or structure.

In some embodiments, nucleobases, sugars and internucleotidic linkages, etc., that can be utilized in provided technologies are described in U.S. Pat. Nos. 9,695,211, 9,605,019, 9,598,458, US 2013/0178612, US 20150211006, US 20170037399, WO 2017/015555, WO 2017/062862, WO 2017/160741, WO 2017/192664, WO 2017/192679, WO 2017/210647, WO 2018/223056. WO 2018/237194, WO 2019/055951, WO 2019/200185, and/or WO 2019/217784, the nucleobases, sugars and internucleotidic linkages of each of which is independently incorporated herein by reference. In some embodiments, various useful technologies (e.g., nucleobases, sugars, internucleotidic linkages, stereochemistry, and patterns thereof, base sequences, oligonucleotides, compositions, methods, etc.) are described in 62/776,432, WO 2019/200185, and WO 2019/217784, each of which is independently incorporated herein by reference.

### Dystrophin

In some embodiments, the present disclosure provides technologies, e.g., DMD oligonucleotides, compositions, methods, etc., related to the dystrophin (DMD) gene or a product encoded thereby (a DMD transcript, a protein (e.g., various variants of the dystrophin protein), etc.).

In some embodiments, the present disclosure provides technologies, including DMD oligonucleotides and compositions and methods of use thereof, for treatment of muscular dystrophy, including but not limited to, Duchenne Muscular Dystrophy (also abbreviated as DMD) and Becker Muscular Dystrophy (BMD). In some embodiments, DMD comprises one or more mutations. In some embodiments, such mutations are associated with reduced biological functions of dystrophin protein in a subject suffering from or susceptible to muscular dystrophy.

In some embodiments, the dystrophin (DMD) gene or a product thereof, or a variant or portion thereof, may be referred to as DMD, BMD, CMD3B, DXS142, DXS164, DXS206, DXS230, DXS239, DXS268, DXS269, DXS270, DXS272, MRX85, or dystrophin; External IDs: OMIM: 300377 MGI: 94909; HomoloGene: 20856; GeneCards: DMD; In Human: Entrez: 1756; Ensembl: ENSG⁰⁰⁰⁰⁰¹⁹⁸⁹⁴⁷; UniProt: P11532; RefSeq (mRNA): NM_000109; NM_004006; NM_004007; NM_004009; NM_004010; RefSeq (protein): NP_000100; NP_003997; NP_004000: NP_004001; NP_004002: Location (UCSC): Chr X: 31.1-33.34 Mb; In Mouse: Entrez: 13405; Ensembl: ENSMUSG⁰⁰⁰⁰⁰⁰⁴⁵¹⁰³; UniProt: P11531; RefSeq (mRNA): NM_007868; NM_001314034; NM_001314035; NM_001314036; NM_001314037; RefSeq (protein): NP_001300963; NP_001300964; NP_001300965; NP_001300966; NP_001300967; Location (UCSC): Chr X: 82.95-85.21 Mb.

The DMD gene reportedly contains 79 exons distributed over 2.3 million bp of genetic real estate on the X chromosome; however, only approximately 14,000 bp (<1%) is reported to be used for translation into protein (coding sequence). It is reported that about 99.5% of the genetic sequence, the intronic sequences, is spliced out of the 2.3 million bp initial heteronuclear RNA DMD transcript to provide a mature 14,000 bp mRNA that includes all key information for dystrophin protein production. In some embodiments, patients with DMD have mutation(s) in the DMD gene that prevent the appropriate construction of the wild-type DMD mRNA and/or the production of the wild-type dystrophin protein, and patients with DMD often show marked dystrophin deficiency in their muscle.

In some embodiments, a dystrophin DMD transcript, e.g., mRNA, or protein encompasses those related to or produced from alternative splicing. For example, sixteen alternative DMD transcripts of the dystrophin gene were reported following an analysis of splicing patterns of the DMD gene in skeletal muscle, brain and heart tissues. Sironi et al. 2002 FEBS Letters 517: 163-166.

It is reported that dystrophin has several isoforms. In some embodiments, dystrophin refers to a specific isoform. At least three full-length dystrophin isoforms have been reported, each controlled by a tissue-specific promoter. Klamut et al. 1990 Mol. Cell. Biol. 10: 193-205; Nudel et al. 1989 Nature 337: 76-78; Gorecki et al. 1992 Hum. Mol. Genet. 1: 505-510. The muscle isoform is reportedly mainly expressed in skeletal muscle but also in smooth and cardiac muscles [Bies, R. D., Phelps, S. F., Cortez, M. D., Roberts, R., Caskey, C. T. and Chamberlain. J. S. 1992 Nucleic Acids Res. 20: 1725-1731], the brain dystrophin is reportedly specific for cortical neurons but can also be detected in heart and cerebellar neurons, while the Purkinje-cell type reportedly accounts for nearly all cerebellar dystrophin [Gorecki et al. 1992 Hum. Mol. Genet. 1: 505-510]. Alternative splicing reportedly provides a means for dystrophin diversification: the 3′ region of the gene reportedly undergoes alternative splicing resulting in tissue-specific DMD transcripts in brain neurons, cardiac Purkinje fibers, and smooth muscle cells [Bies et al. 1992 Nucleic Acids Res. 20: 1725-1731; and Feener et al. 1989 Nature 338: 509-511] while 12 patterns of alternative splicing have been reported in the 5′ region of the gene in skeletal muscle [Surono et al. 1997 Biochem. Biophys. Res. Commun. 239: 895-899].

In some embodiments, a dystrophin mRNA, gene or protein is a revertant version. Among others, revertant dystrophins were reported in, for example: Hoffman et al. 1990 J. Neurol. Sci. 99:9-25; Klein et al. 1992 Am. J. Hum. Genet. 50: 950-959; and Chelly et al. 1990 Cell 63: 1239-1348; Arahata et al. 1998 Nature 333: 861-863; Bonilla et al. 1988 Cell 54: 447-452; Fanin et al. 1992 Neur. Disord. 2: 41-45; Nicholson et al. 1989 J. Neurol. Sci. 94: 137-146: Shimizu et al. 1988 Proc. Jpn. Acad. Sci. 64: 205-208; Sicinzki et al. 1989 Science 244: 1578-1580; and Sherratt et al. Am. J. Hum. Genet. 53: 1007-1015.

All documents cited herein include supplemental data, if any.

Various mutations in the DMD gene can and/or were reported to cause muscular dystrophy, including some in exon 51.

### Muscular Dystrophy

Compositions comprising one or more DMD oligonucleotides described herein can be used to treat or delay onset of muscular dystrophy, or at least one symptom thereof. In some embodiments, muscular dystrophy (MD) is any of a group of muscle conditions, diseases, or disorders that results in (increasing) weakening and breakdown of skeletal muscles over time. The conditions, diseases, or disorders differ in which muscles are primarily affected, the degree of weakness, when symptoms begin, and how quickly symptoms worsen. Many MD patients will eventually become unable to walk. In many cases musuclar dystrophy is fatal. Some types are also associated with problems in other organs, including the central nervous system. In some embodiments, the muscular dystrophy is Duchenne (Duchenne's) Muscular Dystrophy (DMD) or Becker (Becker's) Muscular Dystrophy (BMD).

In some embodiments, a symptom of Duchenne Muscular Dystrophy is reportedly muscle weakness associated with muscle wasting, with the voluntary muscles being first affected, especially those of the hips, pelvic area, thighs, shoulders, and calves. Muscle weakness can reportedly also occur later, in the arms, neck, and other areas. Calves are reportedly often enlarged. Symptoms reportedly usually appear before age six and may appear in early infancy. Other physical symptoms reportedly are: awkward manner of walking, stepping, or running (in some cases, patients tend to walk on their forefeet, because of an increased calf muscle tone), frequent falls, fatigue, difficulty with motor skills (e.g., running, hopping, jumping), lumbar hyperlordosis, possibly leading to shortening of the hip-flexor muscles, unusual overall posture and/or manner of walking, stepping, or running, muscle contractures of Achilles tendon and hamstrings impair functionality, progressive difficulty walking, muscle fiber deformities, pseudohypertrophy (enlarging) of tongue and calf muscles, higher risk of neurobehavioral disorders (e.g., ADHD), learning disorders (e.g., dyslexia), and non-progressive weaknesses in specific cognitive skills (e.g., short-term verbal memory), which are believed to be the result of absent or dysfunctional dystrophin in the brain, eventual loss of ability to walk (usually by the age of 12), skeletal deformities (including scoliosis in some cases), and trouble getting up from lying or sitting position.

In some embodiments, Becker muscular dystrophy (BMD) is reportedly caused by mutations that give rise to shortened but in-frame DMD transcripts resulting in the production of truncated but partially functional protein(s). Such partially functional protein(s) were reported to retain the critical amino terminal, cysteine rich and C-terminal domains but usually lack elements of the central rod domains which were reported to be of less functional significance. England et al. 1990 Nature, 343, 180-182.

In some embodiments, BMD phenotypes range from mild DMD to virtually asymptomatic, depending on the precise mutation and the level of dystrophin produced. Yin et al. 2008 Hum. Mol. Genet. 17: 3909-3918.

In some embodiments, dystrophy patients with out-of-frame mutations are generally diagnosed with the more severe Duchenne Muscular Dystrophy. and dystrophy patients with in-frame mutations are generally diagnosed with the less severe Becker Muscular Dystrophy. However, a minority of patients with in-frame deletions are diagnosed with Duchenne Muscular Dystrophy, including those with deletion mutations starting or ending in exons 50 or 51, which encode part of the hinge region, such as deletions of exons 47 to 51, 48 to 51, and 49 to 53. Without wishing to be bound by any particular theory, the present disclosure notes that the patient-to-patient variability in disease severity despite the presence of the same exon deletion reportedly may be related to the effect of the specific deletion breakpoints on mRNA splicing efficiency and/or patterns; translation or DMD transcription efficiency after genome rearrangement; and stability or function of the truncated protein structure. Yokota et al. 2009 Arch. Neurol. 66: 32.

### Exon Skipping as a Treatment for Muscular Dystrophy

In some embodiments, a treatment for muscular dystrophy comprises the use of a DMD oligonucleotide which is capable of mediating skipping of Dystrophin (DMD) exon 51. In some embodiments, the present disclosure provides methods for treatment of muscular dystrophy comprising administering to a subject suffering therefrom or susceptible thereto a DMD oligonucleotide, or a composition comprising a DMD oligonucleotide. Particularly, among other things, the present disclosure demonstrates that chirally controlled DMD oligonucleotide/chirally controlled DMD oligonucleotide compositions are unexpectedly effective for modulating exon skipping compared to otherwise identical but non-chirally controlled DMD oligonucleotide/oligonucleotide compositions. In some embodiments, the present disclosure demonstrates incorporation of one or more non-negatively charged internucleotidic linkage into a DMD oligonucleotide can greatly improve delivery and/or overall exon skipping efficiency.

In some embodiments, a treatment for muscular dystrophy employs the use of a DMD oligonucleotide, wherein the DMD oligonucleotide is capable of mediating (e.g., directing) skipping of DMD exon 51. In some embodiments, a DMD oligonucleotide is capable of mediating the skipping of an exon which comprises a mutation (e.g., a frameshift, insertion, deletion, missense, or nonsense mutation, or other mutation), wherein translation of the mRNA with a skipped exon produces a truncated but functional (or largely functional) DMD protein.

In some embodiments, a composition comprising a DMD oligonucleotide is useful for treatment of a Dystrophin-related disorder of the central nervous system. In some embodiments, the present disclosure pertains to a method of treatment of a Dystrophin-related disorder of the central nervous system, wherein the method comprises the step of administering a therapeutically effective amount of a DMD oligonucleotide to a patient suffering from a Dystrophin-related disorder of the central nervous system. In some embodiments, a DMD oligonucleotide is administered outside the central nervous system (as non-limiting examples, intravenously or intramuscularly) to a patient suffering from a Dystrophin-related disorder of the central nervous system, and the DMD oligonucleotide is capable of passing through the blood-brain barrier into the central nervous system. In some embodiments, a DMD oligonucleotide is administered directly into the central nervous system (as non-limiting example, via intrathecal, intraventricular, intracranial, etc., delivery).

In some embodiments, a Dystrophin-related disorder of the central nervous system, or a symptom thereof, can be any one or more of: decreased intelligence, decreased long term memory, decreased short term memory, language impairment, epilepsy, autism spectrum disorder, attention deficit hyperactivity disorder (ADHD), obsessive-compulsive disorder, learning problem, behavioral problem, a decrease in brain volume, a decrease in grey matter volume, lower white matter fractional anisotropy, higher white matter radial diffusivity, an abnormality of skull shape, or a deleterious change in the volume or structure of the hippocampus, globus pallidus, caudate putamen, hypothalamus, anterior commissure, periaqueductal gray, internal capsule, amygdala, corpus callosum, septal nucleus, nucleus accumbens, fimbria, ventricle, or midbrain thalamus. In some embodiments, a patient exhibiting muscle-related symptoms of muscular dystrophy also exhibits symptoms of a Dystrophin-related disorder of the central nervous system.

In some embodiments, a Dystrophin-related disorder of the central nervous system is related to, associated with and/or caused by an abnormality in the level, activity, expression and/or distribution of a gene product of the Dystrophin gene, such as full-length Dystrophin or a smaller isoform of Dystrophin, including, but not limited to, Dp260, Dp140, Dp116, Dp71 or Dp40. In some embodiments, a DMD oligonucleotide is administered into the central nervous system of a muscular dystrophy patient in order to ameliorate one or more systems of a Dystrophin-related disorder of the central nervous system. In some embodiments, a Dystrophin-related disorder of the central nervous system is related to, associated with and/or caused by an abnormality in the level, activity, expression and/or distribution of a gene product of the Dystrophin gene, such as full-length Dystrophin or a smaller isoform of Dystrophin, including, but not limited to, Dp260, Dp140, Dp116, Dp71 or Dp40. In some embodiments, administration of a DMD oligonucleotide to a patient suffering from a Dystrophin-related disorder of the central nervous system increases the level, activity, and/or expression and/or improves the distribution of a gene product of the Dystrophin gene.

In some embodiments, the present disclosure provides technologies for modulating dystrophin pre-mRNA splicing, whereby exon 51 is excised to remove a mutation.

In some embodiments, in a DMD patient, a DMD gene comprises an exon comprising a mutation, and the disorder is at least partially treated by skipping of DMD exon 51.

In some embodiments, in a DMD patient, a DMD gene or DMD transcript has a mutation in an exon(s), which is a missense or nonsense mutation and/or deletion, insertion, inversion, translocation or duplication.

In some embodiments, in a treatment for muscular dystrophy, an exon of DMD (e.g., exon 51) is skipped, wherein the exon encodes a string of amino acids not essential for DMD protein function, or whose skipping can provide a fully or at least partially functional DMD protein.

In some embodiments, in a treatment for muscular dystrophy, a DMD oligonucleotide is capable of mediating skipping of DMD exon 51, thereby creating an mRNA from which can be translated into an artificially internally truncated DMD protein variant which provides at least partially improved or fully restored biological activity.

In some embodiments, an internally truncated DMD protein variant produced from a dystrophin DMD transcript with a skipped exon 51 is more functional than a terminally truncated DMD protein e.g., produced from a dystrophin DMD transcript with an out-of-frame deletion.

In some embodiments, an internally truncated DMD protein variant produced from a dystrophin DMD transcript with a skipped exon 51 is more resistant to nonsense-mediated decay, which can degrade a terminally truncated DMD protein, e.g., produced from a dystrophin DMD transcript with an out-of-frame deletion.

In some embodiments, a treatment for muscular dystrophy employs the use of a DMD oligonucleotide, wherein the DMD oligonucleotide is capable of mediating skipping of DMD exon 51.

In some embodiments, the present disclosure encompasses the recognition that the nature and location of a DMD mutation may be utilized to design an exon-skipping strategy. In some embodiments, if a DMD patient has a mutation in an exon, skipping of the mutated exon can produce an internally truncated (internally shortened) but at least partially functional DMD protein variant.

In some embodiments, a DMD patient has a mutation which alters splicing of a DMD transcript, e.g., by inactivating a site required for splicing, or activating a cryptic site so that it becomes active for splicing, or by creating an alternative (e.g., unnatural) splice site. In some embodiments, such a mutation causes production of proteins with low or no activities. In some embodiments, splicing modulation, e.g., exon skipping, suppression of such a mutation, etc., can be employed to remove or reduce effects of such a mutation. e.g., by restoring proper splicing to produce proteins with restored activities, or producing an internally truncated dystrophin protein variant with improved or restored activities, etc.

In some embodiments, restoring the reading frame can convert an out-of-frame mutation to an in-frame mutation; in some embodiments, in humans, such a change can transform severe Duchenne Muscular Dystrophy into milder Becker Muscular Dystrophy.

In some embodiments, a DMD patient or a patient suspected to have DMD is analyzed for DMD genotype prior to administration of a composition comprising a DMD oligonucleotide.

In some embodiments, a DMD patient or a patient suspected to have DMD is analyzed for DMD phenotype prior to administration of a composition comprising a DMD oligonucleotide.

In some embodiments, a DMD patient is analyzed for genotype and phenotype to determine the relationship of DMD genotype and DMD phenotype prior to administration of a composition comprising a DMD oligonucleotide.

In some embodiments, a patient is genetically verified to have dystrophy prior to administration of a composition comprising a DMD oligonucleotide.

In some embodiments, analysis of DMD genotype or genetic verification of DMD or a patient comprises determining if the patient has one or more deleterious mutations in DMD.

In some embodiments, analysis of DMD genotype or genetic verification of DMD or a patient comprises determining if the patient has one or more deleterious mutations in DMD and/or analyzing DMD splicing and/or detecting splice variants of DMD, wherein a splice variant is produced by an abnormal splicing of DMD.

In some embodiments, analysis of DMD genotype or genetic verification of DMD informs the selection of a composition comprising a DMD oligonucleotide useful for treatment.

In some embodiments, an abnormal or mutant DMD gene or a portion thereof is removed or copied from a patient or a patient's cell(s) or tissue(s) and the abnormal or mutant DMD gene, or a portion thereof comprising the abnormality or mutation, or a copy thereof, is inserted into a cell. In some embodiments, this cell can be used to test various compositions comprising a DMD oligonucleotide to predict if such a composition would be useful as a treatment for the patient. In some embodiments, the cell is a myoblast or myotubule.

In some embodiments, an individual or patient can produce, prior to treatment with a DMD oligonucleotide, one or more splice variants of DMD, often each variant being produced at a very low level. In some embodiments, any appropriate method can be used to detect low levels of splice variants being produced in a patient prior to, during or after administration of a DMD oligonucleotide.

In some embodiments, a patient and/or the tissues thereof are analyzed for production of various splicing variants of a DMD gene prior to administration of a composition comprising a DMD oligonucleotide.

In some embodiments, the present disclosure provides methods for designing a DMD oligonucleotide (e.g., a DMD oligonucleotide capable of mediating skipping of DMD exon 51). In some embodiments, the present disclosure utilizes rationale design described herein and optionally sequence walks to design DMD oligonucleotides, e.g., for testing exon skipping in one or more assays and/or conditions. In some embodiments, an efficacious DMD oligonucleotide is developed following rational design, including using various information of a given biological system.

In some embodiments, in a method for developing DMD oligonucleotides, DMD oligonucleotides are designed to anneal to one or more potential splicing-related motifs and then tested for their ability to mediate exon skipping.

### Example Technologies for Assessing Oligonucleotides and Oligonucleotide Compositions

Various technologies for assessing properties and/or activities of DMD oligonucleotides can be utilized in accordance with the present disclosure, e.g., US 20170037399, WO 2017/015555, WO 2017/015575, WO 2017/192664, WO 2017/062862, WO 2017/192679, WO 2017/210647, etc.

For example, DMD oligonucleotides can be evaluated for their ability to mediate exon skipping in various assays, including in vitro and in vivo assays, in accordance with the present disclosure. In vitro assays can be performed in various test cells described herein or known in the art, including but not limited to, Δ48-50 Patient-Derived Myoblast Cells. In vivo tests can be performed in test animals described herein or known in the art, including but not limited to, a mouse, rat, cat, pig, dog, monkey, or non-human primate.

As non-limiting examples, a number of assays are described below for assessing properties/activities of DMD oligonucleotides. Various other suitable assays are available and may be utilized to assess DMD oligonucleotide properties/activities, including those of DMD oligonucleotides not designed for exon skipping (e.g., for DMD oligonucleotides that may involve RNase H for reducing levels of target DMD transcripts, assays described in US 20170037399, WO 2017/015555, WO 2017/015575, WO 2017/192664, WO 2017/192679, WO 2017/210647, etc.).

A DMD oligonucleotide can be evaluated for its ability to mediate skipping of exon 51 in the Dystrophin RNA, which can be tested, as non-limiting examples, using nested PCR, qRT-PCR, and/or sequencing.

A DMD oligonucleotide can be evaluated for its ability to mediate protein restoration (e.g., production of an internally truncated Dystrophin protein variant lacking the amino acids corresponding to the codons encoded in the skipped exon, which has improved functions compared to proteins (if any) produced prior to exon skipping), which can be evaluated by a number of methods for protein detection and/or quantification, such as western blot, immunostaining, etc. Antibodies to dystrophin are commercially available or if desired, can be developed for desired purposes.

A DMD oligonucleotide can be evaluated for its ability to mediate production of a stable restored protein. Stability of restored protein can be tested, in non-limiting examples, in assays for serum and tissue stability.

A DMD oligonucleotide can be evaluated for its ability to bind protein, such as albumin. Example related technologies include those described, e.g., in WO 2017/015555, WO 2017/015575, etc.

A DMD oligonucleotide can be evaluated for immuno activity, e.g., through assays for cytokine activation, complement activation, TLR9 activity, etc. Example related technologies include those described, e.g., in WO 2017/015555, WO 2017/015575, WO 2017/192679, WO 2017/210647, etc.

In some embodiments, efficacy of a DMD oligonucleotide can be tested, e.g., in in silico analysis and prediction, a cell-free extract, a cell transfected with artificial constructs, an animal such as a mouse with a human Dystrophin transgene or portion thereof, normal and dystrophic human myogenic cell lines, and/or clinical trials. It may be desirable to utilize more than one assay, as normal and dystrophic human myogenic cell lines may sometimes produce different efficacy results under certain conditions (Mitrpant et al. 2009 Mol. Ther. 17: 1418).

In some embodiments, DMD oligonucleotides can be tested in vitro in cells. In some embodiments, testing in vitro in cells involves gymnotic delivery of the DMD oligonucleotide(s), or delivery using a delivery agent or transfectant, many of which are known in the art and may be utilized in accordance with the present disclosure.

In some embodiments, DMD oligonucleotides can be tested in vitro in normal human skeletal muscle cells (hSkMCs). See, for example, Arechavala et al. 2007 Hum. Gene Ther. 18: 798-810.

In some embodiments, DMD oligonucleotides can be tested in a muscle explant from a DMD patient. Muscle explants from DMD patients are reported in, for example, Fletcher et al. 2006 J. Gene Med. 8: 207-216; McClorey et al. 2006 Neur. Dis. 16: 583-590; and Arechavala et al. 2007 Hum. Gene Ther. 18: 798-810.

In some embodiments, cells are or comprise cultured muscle cells from DMD patients. See, for example: Aartsma-Rus et al. 2003 Hum. Mol. Genet. 8: 907-914.

In some embodiments, an individual DMD oligonucleotide may demonstrate experiment-to-experiment variability in its ability to skip exon 51 under certain circumstances. In some embodiments, an individual DMD oligonucleotide can demonstrate variability in its ability to skip exon 51 depending on which cells are used, the growth conditions, and other experimental factors. To control variations, typically DMD oligonucleotides to be tested and control DMD oligonucleotides are assayed under the same or substantially the same conditions.

In vitro experiments also include those conducted with patient-derived myoblasts. Certain results from such experiments were described herein. In certain such experiments, cells were cultured in skeletal growth media to keep them in a dividing/immature myoblast state. The media was then changed to ‘differentiation’ media (containing insulin and 2% horse serum) concurrent with spiking DMD oligonucleotides in the media for dosing. The cells differentiated into myotubes as they were getting dosed for a suitable period of time, e.g., a total of 4d for RNA experiments and 6d for protein experiments (such conditions referenced as ‘0 d pre-differentiation’ (0 d+4 d for RNA, 0 d+6 d for protein)).

Without wishing to be bound by any particular theory, the present disclosure notes that it may be desirable to know if DMD oligonucleotides are able to enter mature myotubes and induce skipping in these cells as well as ‘immature’ cells. In some embodiments, the present disclosure provided assays to test effects of DMD oligonucleotides in myotubes. In some embodiments, a dosing schedule different from the ‘0 d pre-differentiation’ was used, wherein the myoblasts were pre-differentiated into myotubes in differentiation media for several days (4 d or 7 d or 10 d) and then DMD oligonucleotides were administered. Certain related protocols are described in Example 19.

In some embodiments, the present disclosure demonstrated that, in the pre-differentiation experiments, DMD oligonucleotides (excluding those which are PMOs) usually give about the same level of RNA skipping and dystrophin protein restoration, regardless of the number of days cells were cultured in differentiation media prior to dosing. In some embodiments, the present disclosure provides DMD oligonucleotides that may be able to enter and be active in myoblasts and in myotubes. In some embodiments, a DMD oligonucleotide is tested in vitro in Δ45-52 DMD patient cells (also designated D45-52 or del45-52) or Δ52 DMD patient cells (also designated D52 or del52) with 0, 4 or 7 days of pre-differentiation.

In some embodiments, DMD oligonucleotides can be tested in any one or more of various animal models, including non-mammalian and mammalian models; including, as non-limiting examples, Caenorhabditis, Drosophila, zebrafish, mouse, rat, cat, dog and pig. See, for example, a review in McGreevey et al. 2015 Dis. Mod. Mech. 8: 195-213.

Example use of mdx mice is reported in, for example: Lu et al. 2003 Nat. Med. 9: 1009; Jearawiriyapaisarn et al. 2008 Mol. Ther., 16, 1624-1629; Yin et al. 2008 Hum. Mol. Genet., 17, 3909-3918: Wu et al. 2009 Mol. Ther., 17, 864-871: Wu et al. 2008 Proc. Natl Acad. Sci. USA, 105, 14814-14819; Mann et al. 2001 Proc. Nat. Acad. Sci. USA 98: 42-47; and Gebski et al. 2003 Hum. Mol. Gen. 12: 1801-1811.

Efficacy of DMD oligonucleotides can be tested in dogs, such as the Golden Retriever Muscular Dystrophy (GRMD) animal model. Lu et al. 2005 Proc. Natl. Acad. Sci. USA 102:198-203; Alter et al. 2006 Nat. Med. 12:175-7; McClorey et al. 2006 Gene Ther. 13:1373-81; and Yokota et al. 2012 Nucl. Acid Ther. 22: 306.

A DMD oligonucleotide can be evaluated in vivo in a test animal for efficient delivery to various tissues (e.g., skeletal, heart and/or diaphragm muscle): this can be tested, in non-limiting examples, by hybridization ELISA and tests for distribution in animal tissue.

A DMD oligonucleotide can be evaluated in vivo in a test animal for plasma PK; this can be tested, as non-limiting examples, by assaying for AUC (area under the curve) and half-life.

In some embodiments, DMD oligonucleotides can be tested in vivo, via an intramuscular administration a muscle of a test animal.

In some embodiments, DMD oligonucleotides can be tested in vivo, via an intramuscular administration into the gastrocnemius muscle of a test animal.

In some embodiments, DMD oligonucleotides can be tested in vivo, via an intramuscular administration into the gastrocnemius muscle of a mouse.

In some embodiments, DMD oligonucleotides can be tested in vivo, via an intramuscular administration into the gastrocnemius muscle of a mouse model transgenic for the entire human dystrophin locus. See, for example: Bremmer-Bout et al. 2004 Mol. Ther. 10, 232-240.

Additional tests which can be performed to evaluate the efficacy of DMO DMD oligonucleotides include centrally nucleated fiber counts and dystrophin-positive fiber counts, and functional grip strength analysis. See, as non-limiting examples, experimental protocols reported in: Yin et al. 2009 Hum. Mol. Genet. 18: 4405-4414.

Additional methods of testing DMD oligonucleotides include, as non-limiting example, methods reported in: Kinali et al. 2009 Lancet 8: 918; Bertoni et al. 2003 Hum. Mol. Gen. 12: 1087-1099.

### Certain Examples of Oligonucleotides and Compositions

In some embodiments, the present disclosure provides DMD oligonucleotides and/or DMD oligonucleotide compositions that are useful for various purposes, e.g., modulating skipping, reducing levels of DMD transcripts, improving levels of beneficial proteins, treating conditions, diseases and disorders, etc. In some embodiments, the present disclosure provides DMD oligonucleotide compositions with improved properties, e.g., increased skipping of exon 51, reduced toxicities, etc. Among other things, DMD oligonucleotides of the present disclosure comprise chemical modifications, stereochemistry, and/or combinations thereof which can improve various properties and activities of DMD oligonucleotides. Non-limiting examples are listed in Table A1. In some embodiments, a DMD oligonucleotide type is a type as defined by the base sequence, pattern of backbone linkages, pattern of backbone chiral centers and pattern of backbone phosphorus modifications of a DMD oligonucleotide in Table A1, wherein the DMD oligonucleotide comprises at least one chirally controlled internucleotidic linkage (at least one R or S in “Stereochemistry/Linkage”).

In some embodiments, the present disclosure pertains to a DMD oligonucleotide described herein, e.g., in Table A1.

In the following table ID indicates identification or DMD oligonucleotide number; and Description indicates the modified sequence.

TABLE A1
Example Oligonucleotides.

| | | SEQ ID | Naked | Linkage/ |
|---|---|---|---|---|
| ID | Description | NO | Sequence | Stereochemistry |
| WV- | fU * SfC * SfA * SfA * SfG * SfG * SmAfA * SmGfA * SmUfG * | 1 | UCAAGGAAGAU | SSSSS SOSOS |
| 3152 | SmGfC * SfA * SfU * SfU * SfU * SfC * SfU | | GGCAUUUCU | OSOSSSSS S |
| WV- | fU * fC * fA * fA * fGfG * mAfA * mGmA * fU * mGmGfC * fA * fU * | 2 | UCAAGGAAGAU | XXXXO XOXOX |
| 7336 | fU * fU * fC * fU | | GGCAUUUCU | XOOXX XXX X |
| WV- | fC * SfU * SfC * SfC * SfG * SfG * SfU * SfU * SmCfU * SmG * SfA | 3 | CUCCGGUUCUG | SSSSS SSSOSS |
| 9517 | * SmAmGfG * SfU * SfG * SfU * SfU * SfC | | AAGGUGUUC | SOOSSSSS |
| WV- | GTTGCCTCCGGTTCTGAAGGTGTTC | 4 | GTTGCCTCCGG | OOOOO OOOOO |
| 13405 | | | TTCTGAAGGT | OOOOO OOOOO |
| | | | GTTC | OOOO |
| WV- | CTCCGGTTCTGAAGGTGTTC | 5 | CTCCGGTTCTGA | OOOOO OOOOO |
| 13406 | | | AGGTGTTC | OOOOO OOOO |
| WV- | TGCCTCCGGTTCTGAAGGTGTTCTTGTA | 6 | TGCCTCCGGTTC | OOOOO OOOOO |
| 13407 | | | TGAAGGTGTT | OOOOO OOOOO |
| | | | CTTGTA | OOOOO OO |
| WV- | fU * SfC * SfC * SfG * SfG * SfU * SfU * SmCfU * SmG * SfA * | 7 | UCCGGUUCUGA | SSSSS SSOSS |
| 13835 | SmAmGfG * SfU * SfG * SfU * SfU * SfC * SfU | | AGGUGUUCU | SOOSSSSS S |
| WV- | fC * SfU * SfCn001RfC * SfG * SfGn001RfU * SfU * SmCfU * SmG | 8 | CUCCGGUUCUG | SS nR SS nR SSOSS |
| 13864 | * SfA * SmAfG * SfG * SfU * SfGn001RfU * SfU * SfC | | AAGGUGUUC | SOSSS nR SS |
| WV- | fC * SfU * SfCn001RfC * SfG * SfGn001RfU * SfU * SmCfU * SmG | 9 | CUCCGGUUCUG | SS nR SS nR SSOSS |
| 14344 | * SfA * SmAfGfG * SfU * SfGn001RfU * SfU * SfC | | AAGGUGUUC | SOOSS nR SS |
| WV- | fU * SfC * SfAn001fA * SfG * SfGn001mAfA * SmGmA * SfU * | 10 | UCAAGGAAGAU | SS nX SS nX OSOSS |
| 14522 | SmGmGfC * SfA * SfU * SfUn001fU * SfC * SfU | | GGCAUUUCU | OOSSS nX SS |
| WV- | fU * SfC * SfAn001fA * SfG * SfGn001mAfA * SmGmA * SfU * | 11 | UCAAGGAAGAU | SS nX SS nX OSOSS |
| 14523 | SmGmGfCn001fA * SfU * SfUn001fU * SfC * SfU | | GGCAUUUCU | OO nX SS nX SS |
| WV- | fU * SfC * SfCn001RfG * SfG * SfUn001RfU * SmCfU * SmG * SfA | 12 | UCCGGUUCUGA | SS nR SS nR SOSSS |
| 14791 | * SmAmGfG * SfU * SfGn001RfU * SfU * SfC * SfU | | AGGUGUUCU | OOSS nR SSS |
| WV- | fU * SfC * SfAn001fA * SfG * SfG * SmAfA * SmGmA * SfU * | 13 | UCAAGGAAGAU | SS nX SSSOSOS SOO |
| 15860 | SmGmGfCn001fA * SfU * SfUn001fU * SfC * SfU | | GGCAUUUCU | nX SS nX SS |
| WV- | fU * SfC * SfAn001fA * SfG * SfGn001mAfA * SmGmA * SfU * | 14 | UCAAGGAAGAU | SS nX SS nX |
| 15861 | SmGmGfC * SfA * SfU * SfU * SfU * SfC * SfU | | GGCAUUUCU | OSOSSOOSSSSS S |
| WV- | fU * SfC * SfA * SfA * SfG * SfG * SmAfA * SmGmA * SfU * | 15 | UCAAGGAAGAU | SSSSS SOSOS SOO |
| 15862 | SmGmGfCn001fA * SfU * SfUn001fU * SfC * SfU | | GGCAUUUCU | nX SS nX SS |
| WV- | fU * SfC * SfAn001fA * SfG * SfG * SmA * SfA * SmGmA * SfU * | 16 | UCAAGGAAGAU | SS nX SSSSS OSS OO |
| 17859 | SmGmGfCn001fA * SfU * SfUn001fU * SfC * SfU | | GGCAUUUCU | nX SS nX SS |
| WV- | fU * SfC * SfAn001fA * SfG * SfG * SmAfA * SmGmA * SfU * | 17 | UCAAGGAAGAU | SS nX SSSOSOS SOS |
| 17860 | SmGmG * SfCn001fA * SfU * SfUn001fU * SfC * SfU | | GGCAUUUCU | nX SS nX SS |
| WV- | fU * SfC * SfAn001fA * SfG * SfG * SmA * SfA * SmGmA * SfU * | 18 | UCAAGGAAGAU | SS nX SSSSS OSSOS |
| 17861 | SmGmG * SfCn001fA * SfU * SfUn001fU * SfC * SfU | | GGCAUUUCU | nX SS nX SS |
| WV- | fU * SfC * SfAn001fA * SfG * SfG * SfA * SfA * SmGmA * SfU * | 19 | UCAAGGAAGAU | SS nX SSSSS OSSOS |
| 17862 | SmGfG * SfCn001fA * SfU * SfUn001fU * SfC * SfU | | GGCAUUUCU | nX SS nX SS |
| WV- | fU * SfC * SfAn001fA * SfG * SfGn001mA * SfA * SmGmA * SfU * | 20 | UCAAGGAAGAU | SS nX SS nX SSOSS |
| 17863 | SmGmGfC * SfA * SfU * SfUn001fU * SfC * SfU | | GGCAUUUCU | OOSSS nX SS |
| WV- | fU * SfC * SfAn001fA * SfG * SfGn001mAfA * SmGmA * SfU * | 21 | UCAAGGAAGAU | SS nX SS nX OSOSS |
| 17864 | SmGmG * SfC * SfA * SfU * SfUn001fU * SfC * SfU | | GGCAUUUCU | OSS SS nX SS |
| WV- | fU * SfC * SfAn001fA * SfG * SfGn001mA * SfA * SmGmA * SfU * | 22 | UCAAGGAAGAU | SS nX SS nX SSOSS |
| 17865 | SmGmG * SfC * SfA * SfU * SfUn001fU * SfC * SfU | | GGCAUUUCU | OSSSS nX SS |
| WV- | fU * SfC * SfAn001fA * SfG * SfGn001fA * SfA * SmGmA * SfU * | 23 | UCAAGGAAGAU | SS nX SS nX SSOSS |
| 17866 | SmGfG * SfC * SfA * SfU * SfUn001fU * SfC * SfU | | GGCAUUUCU | OSSSS nX SS |
| WV- | fU * SfG * SfAn001fA * SfA * SfUn001fC * SfU * SmG * SfC * SmC | 24 | UGAAAUCUGCC | SS nX SS nX SSSSS |
| 20034 | * SfA * SmG * SfA * SfG * SfC * SfAn001fG * SfG * SfU | | AGAGCAGGU | SSSSS nX SS |
| WV- | fU * SfG * SfAn001fA * SfA * SfUn001fC * SfU * SmG * SfC * SmC | 25 | UGAAAUCUGCC | SS nX SS nX SSSSS |
| 20034 | * SfA * SmG * SfA * SfG * SfC * SfAn001fG * SfG * SfU | | AGAGCAGGU | SSSSS nX SS |
| WV- | fA * SfA * SfUn001fC * SfU * SfGn001fC * SfC * SmA * SfG * SmA | 26 | AAUCUGCCAGA | SS nX SS nX SSSSS |
| 20037 | * SfG * SmC * SfA * SfG * SfG * SfUn001fA * SfC * SfC | | GCAGGUACC | SSSSS nX SS |
| WV- | fC * SfU * SfGn001fC * SfC * SfAn001fG * SfA * SmG * SfC * SmA | 27 | CUGCCAGAGCA | SS nX SS nX SSSSS |
| 20040 | * SfG * SmG * SfU * SfA * SfC * SfCn001fU * SfC * SfC | | GGUACCUCC | SSSSS nX SS |
| WV- | fC * SfC * SfAn001fG * SfA * SfGn001fC * SfA * SmG * SfG * SmU | 28 | CCAGAGCAGGU | SS nX SS nX SSSSS |
| 20043 | * SfA * SmC * SfC * SfU * SfC * SfCn001fA * SfA * SfC | | ACCUCCAAC | SSSSS nX SS |
| WV- | fG * SfA * SfGn001fC * SfA * SfGn001fG * SfU * SmA * SfC * SmC | 29 | GAGCAGGUACC | SS nX SS nX SSSSS |
| 20046 | * SfU * SmC * SfC * SfA * SfA * SfCn001fA * SfU * SfC | | UCCAACAUC | SSSSS nX SS |
| WV- | fC * SfA * SfGn001fG * SfU * SfAn001fC * SfC * SmU * SfC * SmC | 30 | CAGGUACCUCC | SS nX SS nX SSSSS |
| 20049 | * SfA * SmA * SfC * SfA * SfU * SfCn001fA * SfA * SfG | | AACAUCAAG | SSSSS nX SS |
| WV- | fA * SfG * SfGn001fU * SfA * SfCn001fC * SfU * SmC * SfC * SmA | 31 | AGGUACCUCCA | SS nX SS nX SSSSS |
| 20050 | * SfA * SmC * SfA * SfU * SfC * SfAn001fA * SfG * SfG | | ACAUCAAGG | SSSSS nX SS |
| WV- | fG * SfG * SfUn001fA * SfC * SfCn001fU * SfC * SmC * SfA * SmA | 32 | GGUACCUCCAA | SS nX SS nX SSSSS |
| 20051 | * SfC * SmA * SfU * SfC * SfA * SfAn001fG * SfG * SfA | | CAUCAAGGA | SSSSS nX SS |
| WV- | fG * SfU * SfAn001fC * SfC * SfUn001fC * SfC * SmA * SfA * SmC | 33 | GUACCUCCAAC | SS nX SS nX SSSSS |
| 20052 | * SfA * SmU * SfC * SfA * SfA * SfGn001fG * SfA * SfA | | AUCAAGGAA | SSSSS nX SS |
| WV- | fU * SfA * SfCn001fC * SfU * SfCn001fC * SfA * SmA * SfC * SmA | 34 | UACCUCCAACA | SS nX SS nX SSSSS |
| 20053 | * SfU * SmC * SfA * SfA * SfG * SfGn001fA * SfA * SfG | | UCAAGGAAG | SSSSS nX SS |
| WV- | fA * SfC * SfCn001fU * SfC * SfCn001fA * SfA * SmC * SfA * SmU | 35 | ACCUCCAACAU | SS nX SS nX SSSSS |
| 20054 | * SfC * SmA * SfA * SfG * SfG * SfAn001fA * SfG * SfA | | CAAGGAAGA | SSSSS nX SS |
| WV- | fC * SfC * SfUn001fC * SfC * SfAn001fA * SfC * SmA * SfU * SmC | 36 | CCUCCAACAUC | SS nX SS nX SSSSS |
| 20055 | * SfA * SmA * SfG * SfG * SfA * SfAn001fG * SfA * SfU | | AAGGAAGAU | SSSSS nX SS |
| WV- | fC * SfU * SfCn001fC * SfA * SfAn001fC * SfA * SmU * SfC * SmA | 37 | CUCCAACAUCAA | SS nX SS nX SSSSS |
| 20056 | * SfA * SmG * SfG * SfA * SfA * SfGn001fA * SfU * SfG | | GGAAGAUG | SSSSS nX SS |
| WV- | fU * SfC * SfCn001fA * SfA * SfCn001fA * SfU * SmC * SfA * SmA | 38 | UCCAACAUCAA | SS nX SS nX SSSSS |
| 20057 | * SfG * SmG * SfA * SfA * SfG * SfAn001fU * SfG * SfG | | GGAAGAUGG | SSSSS nX SS |
| WV- | fC * SfC * SfAn001fA * SfC * SfAn001fU * SfC * SmA * SfA * SmG | 39 | CCAACAUCAAG | SS nX SS nX SSSSS |
| 20058 | * SfG * SmA * SfA * SfG * SfA * SfUn001fG * SfG * SfC | | GAAGAUGGC | SSSSS nX SS |
| WV- | fC * SfA * SfAn001fC * SfA * SfUn001fC * SfA * SmA * SfG * SmG | 40 | CAACAUCAAGG | SS nX SS nX SSSSS |
| 20059 | * SfA * SmA * SfG * SfA * SfU * SfGn001fG * SfC * SfA | | AAGAUGGCA | SSSSS nX SS |
| WV- | fA * SfA * SfCn001fA * SfU * SfCn001fA * SfA * SmG * SfG * SmA | 41 | AACAUCAAGGAA | SS nX SS nX SSSSS |
| 20060 | * SfA * SmG * SfA * SfU * SfG * SfGn001fC * SfA * SfU | | GAUGGCAU | SSSSS nX SS |
| WV- | fA * SfC * SfAn001fU * SfC * SfAn001fA * SfG * SmG * SfA * SmA | 42 | ACAUCAAGGAA | SS nX SS nX SSSSS |
| 20061 | * SfG * SmA * SfU * SfG * SfG * SfCn001fA * SfU * SfU | | GAUGGCAUU | SSSSS nX SS |
| WV- | fC * SfA * SfUn001fC * SfA * SfAn001fG * SfG * SmA * SfA * SmG | 43 | CAUCAAGGAAG | SS nX SS nX SSSSS |
| 20062 | * SfA * SmU * SfG * SfG * SfC * SfAn001fU * SfU * SfU | | AUGGCAUUU | SSSSS nX SS |
| WV- | fA * SfU * SfCn001fA * SfA * SfGn001fG * SfA * SmA * SfG * SmA | 44 | AUCAAGGAAGA | SS nX SS nX SSSSS |
| 20063 | * SfU * SmG * SfG * SfC * SfA * SfUn001fU * SfU * SfC | | UGGCAUUUC | SSSSS nX SS |
| WV- | fU * SfC * SfAn001fA * SfG * SfGn001fA * SfA * SmG * SfA * SmU | 45 | UCAAGGAAGAU | SS nX SS nX SSSSS |
| 20064 | * SfG * SmG * SfC * SfA * SfU * SfUn001fU * SfC * SfU | | GGCAUUUCU | SSSSS nX SS |
| WV- | fU * SfC * SfAn001fA * SfG * SfGn001fA * SfA * SmG * SfA * SmU | 46 | UCAAGGAAGAU | SS nX SS nX SSSSS |
| 20064 | * SfG * SmG * SfC * SfA * SfU * SfUn001fU * SfC * SfU | | GGCAUUUCU | SSSSS nX SS |
| WV- | fC * SfA * SfAn001fG * SfG * SfAn001fA * SfG * SmA * SfU * SmG | 47 | CAAGGAAGAUG | SS nX SS nX SSSSS |
| 20065 | * SfG * SmC * SfA * SfU * SfU * SfUn001fC * SfU * SfA | | GCAUUUCUA | SSSSS nX SS |
| WV- | fA * SfA * SfGn001fG * SfA * SfAn001fG * SfA * SmU * SfG * SmG | 48 | AAGGAAGAUGG | SS nX SS nX SSSSS |
| 20066 | * SfC * SmA * SfU * SfU * SfU * SfCn001fU * SfA * SfG | | CAUUUCUAG | SSSSS nX SS |
| WV- | fA * SfG * SfGn001fA * SfA * SfGn001fA * SfU * SmG * SfG * SmC | 49 | AGGAAGAUGGC | SS nX SS nX SSSSS |
| 20067 | * SfA * SmU * SfU * SfU * SfC * SfUn001fA * SfG * SfU | | AUUUCUAGU | SSSSS nX SS |
| WV- | fG * SfG * SfAn001fA * SfG * SfAn001fU * SfG * SmG * SfC * SmA | 50 | GGAAGAUGGCA | SS nX SS nX SSSSS |
| 20068 | * SfU * SmU * SfU * SfC * SfU * SfAn001fG * SfU * SfU | | UUUCUAGUU | SSSSS nX SS |
| WV- | fG * SfA * SfAn001fG * SfA * SfUn001fG * SfG * SmC * SfA * SmU | 51 | GAAGAUGGCAU | SS nX SS nX SSSSS |
| 20069 | * SfU * SmU * SfC * SfU * SfA * SfGn001fU * SfU * SfU | | UUCUAGUUU | SSSSS nX SS |
| WV- | fA * SfA * SfGn001fA * SfU * SfGn001fG * SfC * SmA * SfU * SmU | 52 | AAGAUGGCAUU | SS nX SS nX SSSSS |
| 20070 | * SfU * SmC * SfU * SfA * SfG * SfUn001fU * SfU * SfG | | UCUAGUUUG | SSSSS nX SS |
| WV- | fA * SfA * SfGn001fA * SfU * SfGn001fG * SfC * SmA * SfU * SmU | 53 | AAGAUGGCAUU | SS nX SS nX SSSSS |
| 20070 | * SfU * SmC * SfU * SfA * SfG * SfUn001fU * SfU * SfG | | UCUAGUUUG | SSSSS nX SS |
| WV- | fA * SfG * SfAn001fU * SfG * SfGn001fC * SfA * SmU * SfU * SmU | 54 | AGAUGGCAUUU | SS nX SS nX SSSSS |
| 20071 | * SfC * SmU * SfA * SfG * SfU * SfUn001fU * SfG * SfG | | CUAGUUUGG | SSSSS nX SS |
| WV- | fG * SfA * SfUn001fG * SfG * SfCn001fA * SfU * SmU * SfU * SmC | 55 | GAUGGCAUUUC | SS nX SS nX SSSSS |
| 20072 | * SfU * SmA * SfG * SfU * SfU * SfUn001fG * SfG * SfA | | UAGUUUGGA | SSSSS nX SS |
| WV- | fA * SfU * SfGn001fG * SfC * SfAn001fU * SfU * SmU * SfC * SmU | 56 | AUGGCAUUUCU | SS nX SS nX SSSSS |
| 20073 | * SfA * SmG * SfU * SfU * SfU * SfGn001fG * SfA * SfG | | AGUUUGGAG | SSSSS nX SS |
| WV- | fA * SfU * SfGn001fG * SfC * SfAn001fU * SfU * SmU * SfC * SmU | 57 | AUGGCAUUUCU | SS nX SS nX SSSSS |
| 20073 | * SfA * SmG * SfU * SfU * SfU * SfGn001fG * SfA * SfG | | AGUUUGGAG | SSSSS nX SS |
| WV- | fU * SfG * SfGn001fC * SfA * SfUn001fU * SfU * SmC * SfU * SmA | 58 | UGGCAUUUCUA | SS nX SS nX SSSSS |
| 20074 | * SfG * SmU * SfU * SfU * SfG * SfGn001fA * SfG * SfA | | GUUUGGAGA | SSSSS nX SS |
| WV- | fG * SfG * SfCn001fA * SfU * SfUn001fU * SfC * SmU * SfA * SmG | 59 | GGCAUUUCUAG | SS nX SS nX SSSSS |
| 20075 | * SfU * SmU * SfU * SfG * SfG * SfAn001fG * SfA * SfU | | UUUGGAGAU | SSSSS nX SS |
| WV- | fG * SfC * SfAn001fU * SfU * SfUn001fC * SfU * SmA * SfG * SmU | 60 | GCAUUUCUAGU | SS nX SS nX SSSSS |
| 20076 | * SfU * SmU * SfG * SfG * SfA * SfGn001fA * SfU * SfG | | UUGGAGAUG | SSSSS nX SS |
| WV- | fG * SfC * SfAn001fU * SfU * SfUn001fC * SfU * SmA * SfG * SmU | 61 | GCAUUUCUAGU | SS nX SS nX SSSSS |
| 20076 | * SfU * SmU * SfG * SfG * SfA * SfGn001fA * SfU * SfG | | UUGGAGAUG | SSSSS nX SS |
| WV- | fC * SfA * SfUn001fU * SfU * SfCn001fU * SfA * SmG * SfU * SmU | 62 | CAUUUCUAGUU | SS nX SS nX SSSSS |
| 20077 | * SfU * SmG * SfG * SfA * SfG * SfAn001fU * SfG * SfG | | UGGAGAUGG | SSSSS nX SS |
| WV- | fA * SfU * SfUn001fU * SfC * SfUn001fA * SfG * SmU * SfU * SmU | 63 | AUUUCUAGUUU | SS nX SS nX SSSSS |
| 20078 | * SfG * SmG * SfA * SfG * SfA * SfUn001fG * SfG * SfC | | GGAGAUGGC | SSSSS nX SS |
| WV- | fU * SfU * SfUn001fC * SfU * SfAn001fG * SfU * SmU * SfU * SmG | 64 | UUUCUAGUUUG | SS nX SS nX SSSSS |
| 20079 | * SfG * SmA * SfG * SfA * SfU * SfGn001fG * SfC * SfA | | GAGAUGGCA | SSSSS nX SS |
| WV- | fU * SfU * SfCn001fU * SfA * SfGn001fU * SfU * SmU * SfG * SmG | 65 | UUCUAGUUUGG | SS nX SS nX SSSSS |
| 20080 | * SfA * SmG * SfA * SfU * SfG * SfGn001fC * SfA * SfG | | AGAUGGCAG | SSSSS nX SS |
| WV- | fU * SfC * SfUn001fA * SfG * SfUn001fU * SfU * SmG * SfG * SmA | 66 | UCUAGUUUGGA | SS nX SS nX SSSSS |
| 20081 | * SfG * SmA * SfU * SfG * SfG * SfCn001fA * SfG * SfU | | GAUGGCAGU | SSSSS nX SS |
| WV- | fC * SfU * SfAn001fG * SfU * SfUn001fU * SfG * SmG * SfA * SmG | 67 | CUAGUUUGGAG | SS nX SS nX SSSSS |
| 20082 | * SfA * SmU * SfG * SfG * SfC * SfAn001fG * SfU * SfU | | AUGGCAGUU | SSSSS nX SS |
| WV- | fU * SfA * SfGn001fU * SfU * SfUn001fG * SfG * SmA * SfG * SmA | 68 | UAGUUUGGAGA | SS nX SS nX SSSSS |
| 20083 | * SfU * SmG * SfG * SfC * SfA * SfGn001fU * SfU * SfU | | UGGCAGUUU | SSSSS nX SS |
| WV- | fA * SfG * SfUn001fU * SfU * SfGn001fG * SfA * SmG * SfA * SmU | 69 | AGUUUGGAGAU | SS nX SS nX SSSSS |
| 20084 | * SfG * SmG * SfC * SfA * SfG * SfUn001fU * SfU * SfC | | GGCAGUUUC | SSSSS nX SS |
| WV- | fG * SfU * SfUn001fU * SfG * SfGn001fA * SfG * SmA * SfU * SmG | 70 | GUUUGGAGAUG | SS nX SS nX SSSSS |
| 20085 | * SfG * SmC * SfA * SfG * SfU * SfUn001fU * SfC * SfC | | GCAGUUUCC | SSSSS nX SS |
| WV- | fU * SfU * SfUn001fG * SfG * SfAn001fG * SfA * SmU * SfG * SmG | 71 | UUUGGAGAUGG | SS nX SS nX SSSSS |
| 20086 | * SfC * SmA * SfG * SfU * SfU * SfUn001fC * SfC * SfU | | CAGUUUCCU | SSSSS nX SS |
| WV- | fU * SfU * SfGn001fG * SfA * SfGn001fA * SfU * SmG * SfG * SmC | 72 | UUGGAGAUGGC | SS nX SS nX SSSSS |
| 20087 | * SfA * SmG * SfU * SfU * SfU * SfCn001fC * SfU * SfU | | AGUUUCCUU | SSSSS nX SS |
| WV- | fU * SfG * SfGn001fA * SfG * SfAn001fU * SfG * SmG * SfC * SmA | 73 | UGGAGAUGGCA | SS nX SS nX SSSSS |
| 20088 | * SfG * SmU * SfU * SfU * SfC * SfCn001fU * SfU * SfA | | GUUUCCUUA | SSSSS nX SS |
| WV- | fG * SfG * SfAn001fG * SfA * SfUn001fG * SfG * SmC * SfA * SmG | 74 | GGAGAUGGCAG | SS nX SS nX SSSSS |
| 20089 | * SfU * SmU * SfU * SfC * SfC * SfUn001fU * SfA * SfG | | UUUCCUUAG | SSSSS nX SS |
| WV- | fG * SfA * SfGn001fA * SfU * SfGn001fG * SfC * SmA * SfG * SmU | 75 | GAGAUGGCAGU | SS nX SS nX SSSSS |
| 20090 | * SfU * SmU * SfC * SfC * SfU * SfUn001fA * SfG * SfU | | UUCCUUAGU | SSSSS nX SS |
| WV- | fA * SfG * SfAn001fU * SfG * SfGn001fC * SfA * SmG * SfU * SmU | 76 | AGAUGGCAGUU | SS nX SS nX SSSSS |
| 20091 | * SfU * SmC * SfC * SfU * SfU * SfAn001fG * SfU * SfA | | UCCUUAGUA | SSSSS nX SS |
| WV- | fG * SfA * SfUn001fG * SfG * SfCn001fA * SfG * SmU * SfU * SmU | 77 | GAUGGCAGUUU | SS nX SS nX SSSSS |
| 20092 | * SfC * SmC * SfU * SfU * SfA * SfGn001fU * SfA * SfA | | CCUUAGUAA | SSSSS nX SS |
| WV- | fA * SfU * SfGn001fG * SfC * SfAn001fG * SfU * SmU * SfU * SmC | 78 | AUGGCAGUUUC | SS nX SS nX SSSSS |
| 20093 | * SfC * SmU * SfU * SfA * SfG * SfUn001fA * SfA * SfC | | CUUAGUAAC | SSSSS nX SS |
| WV- | fU * SfG * SfGn001fC * SfA * SfGn001fU * SfU * SmU * SfC * SmC | 79 | UGGCAGUUUCC | SS nX SS nX SSSSS |
| 20094 | * SfU * SmU * SfA * SfG * SfU * SfAn001fA * SfC * SfC | | UUAGUAACC | SSSSS nX SS |
| WV- | fG * SfG * SfCn001fA * SfG * SfUn001fU * SfU * SmC * SfC * SmU | 80 | GGCAGUUUCCU | SS nX SS nX SSSSS |
| 20095 | * SfU * SmA * SfG * SfU * SfA * SfAn001fC * SfC * SfA | | UAGUAACCA | SSSSS nX SS |
| WV- | fG * SfC * SfAn001fG * SfU * SfUn001fU * SfC * SmC * SfU * SmU | 81 | GCAGUUUCCUU | SS nX SS nX SSSSS |
| 20096 | * SfA * SmG * SfU * SfA * SfA * SfCn001fC * SfA * SfC | | AGUAACCAC | SSSSS nX SS |
| WV- | fC * SfA * SfGn001fU * SfU * SfUn001fC * SfC * SmU * SfU * SmA | 82 | CAGUUUCCUUA | SS nX SS nX SSSSS |
| 20097 | * SfG * SmU * SfA * SfA * SfC * SfCn001fA * SfC * SfA | | GUAACCACA | SSSSS nX SS |
| WV- | fA * SfG * SfUn001fU * SfU * SfCn001fC * SfU * SmU * SfA * SmG | 83 | AGUUUCCUUAG | SS nX SS nX SSSSS |
| 20098 | * SfU * SmA * SfA * SfC * SfC * SfAn001fC * SfA * SfG | | UAACCACAG | SSSSS nX SS |
| WV- | fG * SfU * SfUn001fU * SfC * SfCn001fU * SfU * SmA * SfG * SmU | 84 | GUUUCCUUAGU | SS nX SS nX SSSSS |
| 20099 | * SfA * SmA * SfC * SfC * SfA * SfCn001fA * SfG * SfG | | AACCACAGG | SSSSS nX SS |
| WV- | fU * SfU * SfUn001fC * SfC * SfUn001fU * SfA * SmG * SfU * SmA | 85 | UUUCCUUAGUA | SS nX SS nX SSSSS |
| 20100 | * SfA * SmC * SfC * SfA * SfC * SfAn001fG * SfG * SfU | | ACCACAGGU | SSSSS nX SS |
| WV- | fU * SfU * SfCn001fC * SfU * SfUn001fA * SfG * SmU * SfA * SmA | 86 | UUCCUUAGUAA | SS nX SS nX SSSSS |
| 20101 | * SfC * SmC * SfA * SfC * SfA * SfGn001fG * SfU * SfU | | CCACAGGUU | SSSSS nX SS |
| WV- | fU * SfC * SfCn001fU * SfU * SfAn001fG * SfU * SmA * SfA * SmC | 87 | UCCUUAGUAAC | SS nX SS nX SSSSS |
| 20102 | * SfC * SmA * SfC * SfA * SfG * SfGn001fU * SfU * SfG | | CACAGGUUG | SSSSS nX SS |
| WV- | fC * SfC * SfUn001fU * SfA * SfGn001fU * SfA * SmA * SfC * SmC | 88 | CCUUAGUAACC | SS nX SS nX SSSSS |
| 20103 | * SfA * SmC * SfA * SfG * SfG * SfUn001fU * SfG * SfU | | ACAGGUUGU | SSSSS nX SS |
| WV- | fC * SfU * SfUn001fA * SfG * SfUn001fA * SfA * SmC * SfC * SmA | 89 | CUUAGUAACCA | SS nX SS nX SSSSS |
| 20104 | * SfC * SmA * SfG * SfG * SfU * SfUn001fG * SfU * SfG | | CAGGUUGUG | SSSSS nX SS |
| WV- | fU * SfU * SfAn001fG * SfU * SfAn001fA * SfC * SmC * SfA * SmC | 90 | UUAGUAACCAC | SS nX SS nX SSSSS |
| 20105 | * SfA * SmG * SfG * SfU * SfU * SfGn001fU * SfG * SfU | | AGGUUGUGU | SSSSS nX SS |
| WV- | fU * SfA * SfGn001fU * SfA * SfAn001fC * SfC * SmA * SfC * SmA | 91 | UAGUAACCACA | SS nX SS nX SSSSS |
| 20106 | * SfG * SmG * SfU * SfU * SfG * SfUn001fG * SfU * SfC | | GGUUGUGUC | SSSSS nX SS |
| WV- | fA * SfG * SfUn001fA * SfA * SfCn001fC * SfA * SmC * SfA * SmG | 92 | AGUAACCACAG | SS nX SS nX SSSSS |
| 20107 | * SfG * SmU * SfU * SfG * SfU * SfGn001fU * SfC * SfA | | GUUGUGUCA | SSSSS nX SS |
| WV- | fG * SfU * SfAn001fA * SfC * SfCn001fA * SfC * SmA * SfG * SmG | 93 | GUAACCACAGG | SS nX SS nX SSSSS |
| 20108 | * SfU * SmU * SfG * SfU * SfG * SfUn001fC * SfA * SfC | | UUGUGUCAC | SSSSS nX SS |
| WV- | fU * SfA * SfAn001fC * SfC * SfAn001fC * SfA * SmG * SfG * SmU | 94 | UAACCACAGGU | SS nX SS nX SSSSS |
| 20109 | * SfU * SmG * SfU * SfG * SfU * SfCn001fA * SfC * SfC | | UGUGUCACC | SSSSS nX SS |
| WV- | fA * SfA * SfCn001fC * SfA * SfCn001fA * SfG * SmG * SfU * SmU | 95 | AACCACAGGUU | SS nX SS nX SSSSS |
| 20110 | * SfG * SmU * SfG * SfU * SfC * SfAn001fC * SfC * SfA | | GUGUCACCA | SSSSS nX SS |
| WV- | fA * SfC * SfCn001fA * SfC * SfAn001fG * SfG * SmU * SfU * SmG | 96 | ACCACAGGUUG | SS nX SS nX SSSSS |
| 20111 | * SfU * SmG * SfU * SfC * SfA * SfCn001fC * SfA * SfG | | UGUCACCAG | SSSSS nX SS |
| WV- | fC * SfC * SfAn001fC * SfA * SfGn001fG * SfU * SmU * SfG * SmU | 97 | CCACAGGUUGU | SS nX SS nX SSSSS |
| 20112 | * SfG * SmU * SfC * SfA * SfC * SfCn001fA * SfG * SfA | | GUCACCAGA | SSSSS nX SS |
| WV- | fC * SfA * SfCn001fA * SfG * SfGn001fU * SfU * SmG * SfU * SmG | 98 | CACAGGUUGUG | SS nX SS nX SSSSS |
| 20113 | * SfU * SmC * SfA * SfC * SfC * SfAn001fG * SfA * SfG | | UCACCAGAG | SSSSS nX SS |
| WV- | fA * SfC * SfAn001fG * SfG * SfUn001fU * SfG * SmU * SfG * SmU | 99 | ACAGGUUGUGU | SS nX SS nX SSSSS |
| 20114 | * SfC * SmA * SfC * SfC * SfA * SfGn001fA * SfG * SfU | | CACCAGAGU | SSSSS nX SS |
| WV- | fC * SfA * SfGn001fG * SfU * SfUn001fG * SfU * SmG * SfU * SmC | 100 | CAGGUUGUGUC | SS nX SS nX SSSSS |
| 20115 | * SfA * SmC * SfC * SfA * SfG * SfAn001fG * SfU * SfA | | ACCAGAGUA | SSSSS nX SS |
| WV- | fA * SfG * SfGn001fU * SfU * SfGn001fU * SfG * SmU * SfC * SmA | 101 | AGGUUGUGUCA | SS nX SS nX SSSSS |
| 20116 | * SfC * SmC * SfA * SfG * SfA * SfGn001fU * SfA * SfA | | CCAGAGUAA | SSSSS nX SS |
| WV- | fG * SfG * SfUn001fU * SfG * SfUn001fG * SfU * SmC * SfA * SmC | 102 | GGUUGUGUCAC | SS nX SS nX SSSSS |
| 20117 | * SfC * SmA * SfG * SfA * SfG * SfUn001fA * SfA * SfC | | CAGAGUAAC | SSSSS nX SS |
| WV- | fG * SfU * SfUn001fG * SfU * SfGn001fU * SfC * SmA * SfC * SmC | 103 | GUUGUGUCACC | SS nX SS nX SSSSS |
| 20118 | * SfA * SmG * SfA * SfG * SfU * SfAn001fA * SfC * SfA | | AGAGUAACA | SSSSS nX SS |
| WV- | fU * SfU * SfGn001fU * SfG * SfUn001fC * SfA * SmC * SfC * SmA | 104 | UUGUGUCACCA | SS nX SS nX SSSSS |
| 20119 | * SfG * SmA * SfG * SfU * SfA * SfAn001fC * SfA * SfG | | GAGUAACAG | SSSSS nX SS |
| WV- | fU * SfG * SfUn001fG * SfU * SfCn001fA * SfC * SmC * SfA * SmG | 105 | UGUGUCACCAG | SS nX SS nX SSSSS |
| 20120 | * SfA * SmG * SfU * SfA * SfA * SfCn001fA * SfG * SfU | | AGUAACAGU | SSSSS nX SS |
| WV- | fG * SfU * SfGn001fU * SfC * SfAn001fC * SfC * SmA * SfG * SmA | 106 | GUGUCACCAGA | SS nX SS nX SSSSS |
| 20121 | * SfG * SmU * SfA * SfA * SfC * SfAn001fG * SfU * SfC | | GUAACAGUC | SSSSS nX SS |
| WV- | fU * SfG * SfUn001fC * SfA * SfCn001fC * SfA * SmG * SfA * SmG | 107 | UGUCACCAGAG | SS nX SS nX SSSSS |
| 20122 | * SfU * SmA * SfA * SfC * SfA * SfGn001fU * SfC * SfU | | UAACAGUCU | SSSSS nX SS |
| WV- | fG * SfU * SfCn001fA * SfC * SfCn001fA * SfG * SmA * SfG * SmU | 108 | GUCACCAGAGU | SS nX SS nX SSSSS |
| 20123 | * SfA * SmA * SfC * SfA * SfG * SfUn001fC * SfU * SfG | | AACAGUCUG | SSSSS nX SS |
| WV- | fU * SfC * SfAn001fC * SfC * SfAn001fG * SfA * SmG * SfU * SmA | 109 | UCACCAGAGUA | SS nX SS nX SSSSS |
| 20124 | * SfA * SmC * SfA * SfG * SfU * SfCn001fU * SfG * SfA | | ACAGUCUGA | SSSSS nX SS |
| WV- | fC * SfA * SfCn001fC * SfA * SfGn001fA * SfG * SmU * SfA * SmA | 110 | CACCAGAGUAA | SS nX SS nX SSSSS |
| 20125 | * SfC * SmA * SfG * SfU * SfC * SfUn001fG * SfA * SfG | | CAGUCUGAG | SSSSS nX SS |
| WV- | fA * SfC * SfCn001fA * SfG * SfAn001fG * SfU * SmA * SfA * SmC | 111 | ACCAGAGUAAC | SS nX SS nX SSSSS |
| 20126 | * SfA * SmG * SfU * SfC * SfU * SfGn001fA * SfG * SfU | | AGUCUGAGU | SSSSS nX SS |
| WV- | fC * SfC * SfAn001fG * SfA * SfGn001fU * SfA * SmA * SfC * SmA | 112 | CCAGAGUAACA | SS nX SS nX SSSSS |
| 20127 | * SfG * SmU * SfC * SfU * SfG * SfAn001fG * SfU * SfA | | GUCUGAGUA | SSSSS nX SS |
| WV- | fC * SfA * SfGn001fA * SfG * SfUn001fA * SfA * SmC * SfA * SmG | 113 | CAGAGUAACAG | SS nX SS nX SSSSS |
| 20128 | * SfU * SmC * SfU * SfG * SfA * SfGn001fU * SfA * SfG | | UCUGAGUAG | SSSSS nX SS |
| WV- | fA * SfG * SfAn001fG * SfU * SfAn001fA * SfC * SmA * SfG * SmU | 114 | AGAGUAACAGU | SS nX SS nX SSSSS |
| 20129 | * SfC * SmU * SfG * SfA * SfG * SfUn001fA * SfG * SfG | | CUGAGUAGG | SSSSS nX SS |
| WV- | fG * SfA * SfGn001fU * SfA * SfAn001fC * SfA * SmG * SfU * SmC | 115 | GAGUAACAGUC | SS nX SS nX SSSSS |
| 20130 | * SfU * SmG * SfA * SfG * SfU * SfAn001fG * SfG * SfA | | UGAGUAGGA | SSSSS nX SS |
| WV- | fA * SfG * SfUn001fA * SfA * SfCn001fA * SfG * SmU * SfC * SmU | 116 | AGUAACAGUCU | SS nX SS nX SSSSS |
| 20131 | * SfG * SmA * SfG * SfU * SfA * SfGn001fG * SfA * SfG | | GAGUAGGAG | SSSSS nX SS |
| WV- | fG * SfU * SfAn001fA * SfC * SfAn001fG * SfU * SmC * SfU * SmG | 117 | GUAACAGUCUG | SS nX SS nX SSSSS |
| 20132 | * SfA * SmG * SfU * SfA * SfG * SfGn001fA * SfG * SfC | | AGUAGGAGC | SSSSS nX SS |
| WV- | fU * SfA * SfAn001fC * SfA * SfGn001fU * SfC * SmU * SfG * SmA | 118 | UAACAGUCUGA | SS nX SS nX SSSSS |
| 20133 | * SfG * SmU * SfA * SfG * SfG * SfAn001fG * SfC * SfU | | GUAGGAGCU | SSSSS nX SS |
| WV- | fA * SfA * SfCn001fA * SfG * SfUn001fC * SfU * SmG * SfA * SmG | 119 | AACAGUCUGAG | SS nX SS nX SSSSS |
| 20134 | * SfU * SmA * SfG * SfG * SfA * SfGn001fC * SfU * SfA | | UAGGAGCUA | SSSSS nX SS |
| WV- | fA * SfC * SfAn001fG * SfU * SfCn001fU * SfG * SmA * SfG * SmU | 120 | ACAGUCUGAGU | SS nX SS nX SSSSS |
| 20135 | * SfA * SmG * SfG * SfA * SfG * SfCn001fU * SfA * SfA | | AGGAGCUAA | SSSSS nX SS |
| WV- | fC * SfA * SfGn001fU * SfC * SfUn001fG * SfA * SmG * SfU * SmA | 121 | CAGUCUGAGUA | SS nX SS nX SSSSS |
| 20136 | * SfG * SmG * SfA * SfG * SfC * SfUn001fA * SfA * SfA | | GGAGCUAAA | SSSSS nX SS |
| WV- | fA * SfG * SfUn001fC * SfU * SfGn001fA * SfG * SmU * SfA * SmG | 122 | AGUCUGAGUAG | SS nX SS nX SSSSS |
| 20137 | * SfG * SmA * SfG * SfC * SfU * SfAn001fA * SfA * SfA | | GAGCUAAAA | SSSSS nX SS |
| WV- | fG * SfU * SfCn001fU * SfG * SfAn001fG * SfU * SmA * SfG * SmG | 123 | GUCUGAGUAGG | SS nX SS nX SSSSS |
| 20138 | * SfA * SmG * SfC * SfU * SfA * SfAn001fA * SfA * SfU | | AGCUAAAAU | SSSSS nX SS |
| WV- | fU * SfC * SfUn001fG * SfA * SfGn001fU * SfA * SmG * SfG * SmA | 124 | UCUGAGUAGGA | SS nX SS nX SSSSS |
| 20139 | * SfG * SmC * SfU * SfA * SfA * SfAn001fA * SfU * SfA | | GCUAAAAUA | SSSSS nX SS |
| WV- | fC * SfU * SfGn001fA * SfG * SfUn001fA * SfG * SmG * SfA * SmG | 125 | CUGAGUAGGAG | SS nX SS nX SSSSS |
| 20140 | * SfC * SmU * SfA * SfA * SfA * SfAn001fU * SfA * SfU | | CUAAAAUAU | SSSSS nX SS |
| WV- | fG * SfG * SfUn001fA * SfA * SfGn001fU * SfU * SmC * SfU * SmG | 126 | GGUAAGUUCUG | SSnXSSnXSSSS |
| 20011 | * SfU * SmC * SfC * SfA * SfA * SfGn001fC * SfC * SfC | | UCCAAGCCC | SSSSSSnXSS |
| WV- | fG * SfU * SfAn001fC * SfC * SfUn001fC * SfC * SmA * SfA * SmC | 127 | GUACCUCCAAC | SSnXSSnXSSSS |
| 20052 | * SfA * SmU * SfC * SfA * SfA * SfGn001fG * SfA * SfA | | AUCAAGGAA | SSSSSSnXSS |
| WV- | fC * SfA * SfAn001fC * SfA * SfUn001fC * SfA * SmA * SfG * SmG | 128 | CAACAUCAAGG | SSnXSSnXSSSS |
| 20059 | * SfA * SmA * SfG * SfA * SfU * SfGn001fG * SfC * SfA | | AAGAUGGCA | SSSSSSnXSS |
| WV- | fG * SfA * SfUn001fG * SfG * SfCn001fA * SfU * SmU * SfU * SmC | 129 | GAUGGCAUUUC | SSnXSSnXSSSSS |
| 20072 | * SfU * SmA * SfG * SfU * SfU * SfUn001fG * SfG * SfA | | UAGUUUGGA | SSSSSnXSS |
| WV- | fA * SfU * SfGn001fG * SfC * SfAn001fU * SfU * SmU * SfC * SmU | 130 | AUGGCAUUUCU | SSnXSSnXSSSS |
| 20073 | * SfA * SmG * SfU * SfU * SfU * SfGn001fG * SfA * SfG | | AGUUUGGAG | SSSSSSnXSS |
| WV- | fU * SfG * SfGn001fC * SfA * SfUn001fU * SfU * SmC * SfU * SmA | 131 | UGGCAUUUCUA | SSnXSSnXSSSS |
| 20074 | * SfG * SmU * SfU * SfU * SfG * SfGn001fA * SfG * SfA | | GUUUGGAGA | SSSSSSnXSS |
| WV- | fG * SfG * SfCn001fA * SfU * SfUn001fU * SfC * SmU * SfA * SmG | 132 | GGCAUUUCUAG | SSnXSSnXSSSS |
| 20075 | * SfU * SmU * SfU * SfG * SfG * SfAn001fG * SfA * SfU | | UUUGGAGAU | SSSSSSnXSS |
| WV- | fG * SfC * SfAn001fU * SfU * SfUn001fC * SfU * SmA * SfG * SmU | 133 | GCAUUUCUAGU | SSnXSSnXSSSS |
| 20076 | * SfU * SmU * SfG * SfG * SfA * SfGn001fA * SfU * SfG | | UUGGAGAUG | SSSSSSnXSS |
| WV- | fG * SfC * SfAn001fG * SfU * SfUn001fU * SfC * SmC * SfU * SmU | 134 | GCAGUUUCCUU | SSnXSSnXSSSS |
| 20096 | * SfA * SmG * SfU * SfA * SfA * SfCn001fC * SfA * SfC | | AGUAACCAC | SSSSSSnXSS |
| WV- | fC * SfA * SfGn001fU * SfU * SfUn001fC * SfC * SmU * SfU * SmA | 135 | CAGUUUCCUUA | SSnXSSnXSSSS |
| 20097 | * SfG * SmU * SfA * SfA * SfC * SfCn001fA * SfC * SfA | | GUAACCACA | SSSSSSnXSS |
| WV- | fU * SfU * SfCn001fC * SfU * SfUn001fA * SfG * SmU * SfA * SmA | 136 | UUCCUUAGUAA | SSnXSSnXSSSS |
| 20101 | * SfC * SmC * SfA * SfC * SfA * SfGn001fG * SfU * SfU | | CCACAGGUU | SSSSSSnXSS |
| WV- | fU * SfU * SfGn001fU * SfG * SfUn001fC * SfA * SmC * SfC * SmA | 137 | UUGUGUCACCA | SSnXSSnXSSSS |
| 20119 | * SfG * SmA * SfG * SfU * SfA * SfAn001fC * SfA * SfG | | GAGUAACAG | SSSSSSnXSS |
| WV- | fC * SfAn001fG * SfU * SfUn001fU * SfC * SmC * SfU * SmU * SfA | 138 | CAGUUUCCUUA | SnXSSnXSSSSSSSSS |
| 30233 | * SmG * SfU * SfA * SfA * SfCn001fC * SfA * SfC | | GUAACCAC | SnXSS |
| WV- | fG * SfU * SfUn001fU * SfC * SfCn001fU * SfU * SmA * SfG * SmU | 139 | GUUUCCUUAGU | SSnXSSnXSSSSSSSS |
| 30234 | * SfA * SmA * SfC * SfC * SfA * SfCn001fA * SfG | | AACCACAG | SSnXS |
| WV- | fG * SfU * SfAn001fA * SfG * SmU * SfU * SmC * SfU * SmG * SfU | 140 | GUAAGUUCUGU | SSnXSSSSSSSSSSnX |
| 30235 | * SfC * SfC * SfAn001fA * SfG * SfC | | CCAAGC | SS |
| WV- | fGn001fU * SfAn001fA * SfG * SmU * SfU * SmC * SfU * SmG * | 141 | GUAAGUUCUGU | nXSnXSSSSSSSSSSn |
| 30236 | SfU * SfC * SfC * SfAn001fA * SfG * SfC | | CCAAGC | XSS |
| WV- | fU * SfA * SfCn001fC * SfU * SfCn001fC * SfA * SmA * SfC * SmA | 142 | UACCUCCAACA | SSnXSSnXSSSSSSSS |
| 30285 | * SfU * SmC * SfA * SfA * SfG * SfGn001fA * SfA | | UCAAGGAA | SSnXS |
| WV- | fG * SfU * SfA * SfC * SfC * SfU * SfC * SfC * SmA * SfA * SmC * | 143 | GUACCUCCAAC | SSSSSSSSSSSSSSSS |
| 31200 | SfA * SmU * SfC * SfA * SfA * SfG * SfG * SfA * SfA | | AUCAAGGAA | SSS |
| WV- | fU * SfG * SfG * SfC * SfA * SfU * SfU * SfU * SmC * SfU * SmA * | 144 | UGGCAUUUCUA | SSSSSSSSSSSSSSSS |
| 31211 | SfG * SmU * SfU * SfU * SfG * SfG * SfA * SfG * SfA | | GUUUGGAGA | SSS |
| WV- | fG * SfG * SfC * SfA * SfU * SfU * SfU * SfC * SmU * SfA * SmG * | 145 | GGCAUUUCUAG | SSSSSSSSSSSSSSSS |
| 31212 | SfU * SmU * SfU * SfG * SfG * SfA * SfG * SfA * SfU | | UUUGGAGAU | SSS |
| WV- | fU * SfG * SfG * SfC * SfA * SfG * SfU * SfU * SmU * SfC * SmC * | 146 | UGGCAGUUUCC | SSSSSSSSSSSSSSSS |
| 31214 | SfU * SmU * SfA * SfG * SfU * SfA * SfA * SfC * SfC | | UUAGUAACC | SSS |
| WV- | fG * SfG * SfUn001RfA * SfA * SfGn001RmUfU * SmCfU * SmGfU | 147 | GGUAAGUUCUG | SSn RSSn ROSOSOSO |
| 31537 | * SmCfC * SfA * SfA * SfGn001RfC * SfC * SfC | | UCCAAGCCC | SSSnRSS |
| WV- | fG * SfU * SfAn001RfC * SfC * SfUn001RmCfC * SmAfA * SmCfA * | 148 | GUACCUCCAAC | SSn RSSn ROSOSOSO |
| 31538 | SmUfC * SfA * SfA * SfGn001RfG * SfA * SfA | | AUCAAGGAA | SSSnRSS |
| WV- | fC * SfA * SfAn001RfC * SfA * SfUn001RmCfA * SmAfG * SmGfA * | 149 | CAACAUCAAGG | SSn RSSn ROSOSOSO |
| 31539 | SmAfG * SfA * SfU * SfGn001RfG * SfC * SfA | | AAGAUGGCA | SSSnRSS |
| WV- | fA * SfU * SfGn001RfG * SfC * SfAn001RmUfU * SmUfC * SmUfA | 150 | AUGGCAUUUCU | SSn RSSn ROSOSOSO |
| 31540 | * SmGfU * SfU * SfU * SfGn001RfG * SfA * SfG | | AGUUUGGAG | SSSnRSS |
| WV- | fU * SfG * SfGn001RfC * SfA * SfUn001RmUfU * SmCfU * SmAfG | 151 | UGGCAUUUCUA | SSn RSSn ROSOSOSO |
| 31541 | * SmUfU * SfU * SfG * SfGn001RfA * SfG * SfA | | GUUUGGAGA | SSSnRSS |
| WV- | fG * SfG * SfCn001RfA * SfU * SfUn001RmUfC * SmUfA * SmGfU | 152 | GGCAUUUCUAG | SSn RSSn ROSOSOSO |
| 31542 | * SmUfU * SfG * SfG * SfAn001RfG * SfA * SfU | | UUUGGAGAU | SSSnRSS |
| WV- | fG * SfC * SfAn001RfU * SfU * SfUn001RmCfU * SmAfG * SmUfU | 153 | GCAUUUCUAGU | SSn RSSn ROSOSOSO |
| 31543 | * SmUfG * SfG * SfA * SfGn001RfA * SfU * SfG | | UUGGAGAUG | SSSnRSS |
| WV- | fU * SfG * SfGn001RfC * SfA * SfGn001RmUfU * SmUfC * SmCfU | 154 | UGGCAGUUUCC | SSn RSSn ROSOSOSO |
| 31544 | * SmUfA * SfG * SfU * SfAn001RfA * SfC * SfC | | UUAGUAACC | SSSnRSS |
| WV- | fC * SfA * SfGn001RfU * SfU * SfUn001RmCfC * SmUfU * SmAfG | 155 | CAGUUUCCUUA | SSn RSSn ROSOSOSO |
| 31545 | * SmUfA * SfA * SfC * SfCn001RfA * SfC * SfA | | GUAACCACA | SSSnRSS |
| WV- | fA * SfG * SfUn001RfU * SfU * SfCn001RmCfU * SmUfA * SmGfU | 156 | AGUUUCCUUAG | SSn RSSn ROSOSOSO |
| 31546 | * SmAfA * SfC * SfC * SfAn001RfC * SfA * SfG | | UAACCACAG | SSSnRSS |
| WV- | fU * SfU * SfCn001RfC * SfU * SfUn001RmAfG * SmUfA * SmAfC | 157 | UUCCUUAGUAA | SSn RSSn ROSOSOSO |
| 31547 | * SmCfA * SfC * SfA * SfGn001RfG * SfU * SfU | | CCACAGGUU | SSSnRSS |
| WV- | fU * SfU * SfGn001RfU * SfG * SfUn001RmCfA * SmCfC * SmAfG | 158 | UUGUGUCACCA | SSn RSSn ROSOSOSO |
| 31548 | * SmAfG * SfU * SfA * SfAn001RfC * SfA * SfG | | GAGUAACAG | SSSnRSS |
| WV- | fG * SfG * SfUn001RfA * SfA * SfGn001RmUfU * SmCmU * SfG * | 159 | GGUAAGUUCUG | SSnRSSnROSOSSOO |
| 31549 | SmUmCfC * SfA * SfA * SfGn001RfC * SfC * SfC | | UCCAAGCCC | SSSnRSS |
| WV- | fG * SfU * SfAn001RfC * SfC * SfUn001RmCfC * SmAmA * SfC * | 160 | GUACCUCCAAC | SSnRSSnROSOSSOO |
| 31550 | SmAmUfC * SfA * SfA * SfGn001RfG * SfA * SfA | | AUCAAGGAA | SSSnRSS |
| WV- | fC * SfA * SfAn001RfC * SfA * SfUn001RmCfA * SmAmG * SfG * | 161 | CAACAUCAAGG | SSnRSSnROSOSSOO |
| 31551 | SmAmAfG * SfA * SfU * SfGn001RfG * SfC * SfA | | AAGAUGGCA | SSSnRSS |
| WV- | fA * SfU * SfGn001RfG * SfC * SfAn001RmUfU * SmUmC * SfU * | 162 | AUGGCAUUUCU | SSnRSSnROSOSSOO |
| 31552 | SmAmGfU * SfU * SfU * SfGn001RfG * SfA * SfG | | AGUUUGGAG | SSSnRSS |
| WV- | fU * SfG * SfGn001RfC * SfA * SfUn001RmUfU * SmCmU * SfA * | 163 | UGGCAUUUCUA | SSnRSSnROSOSSOO |
| 31553 | SmGmUfU * SfU * SfG * SfGn001RfA * SfG * SfA | | GUUUGGAGA | SSSnRSS |
| WV- | fG * SfG * SfCn001RfA * SfU * SfUn001RmUfC * SmUmA * SfG * | 164 | GGCAUUUCUAG | SSnRSSnROSOSSOO |
| 31554 | SmUmUfU * SfG * SfG * SfAn001RfG * SfA * SfU | | UUUGGAGAU | SSSnRSS |
| WV- | fG * SfC * SfAn001RfU * SfU * SfUn001RmCfU * SmAmG * SfU * | 165 | GCAUUUCUAGU | SSnRSSnROSOSSOO |
| 31555 | SmUmUfG * SfG * SfA * SfGn001RfA * SfU * SfG | | UUGGAGAUG | SSSnRSS |
| WV- | fU * SfG * SfGn001RfC * SfA * SfGn001RmUfU * SmUmC * SfC * | 166 | UGGCAGUUUCC | SSnRSSnROSOSSOO |
| 31556 | SmUmUfA * SfG * SfU * SfAn001RfA * SfC * SfC | | UUAGUAACC | SSSnRSS |
| WV- | fC * SfA * SfGn001RfU * SfU * SfUn001RmCfC * SmUmU * SfA * | 167 | CAGUUUCCUUA | SSnRSSnROSOSSOO |
| 31557 | SmGmUfA * SfA * SfC * SfCn001RfA * SfC * SfA | | GUAACCACA | SSSnRSS |
| WV- | fA * SfG * SfUn001RfU * SfU * SfCn001RmCfU * SmUmA * SfG * | 168 | AGUUUCCUUAG | SSnRSSnROSOSSOO |
| 31558 | SmUmAfA * SfC * SfC * SfAn001RfC * SfA * SfG | | UAACCACAG | SSSnRSS |
| WV- | fU * SfU * SfCn001RfC * SfU * SfUn001RmAfG * SmUmA * SfA * | 169 | UUCCUUAGUAA | SSnRSSnROSOSSOO |
| 31559 | SmCmCfA * SfC * SfA * SfGn001RfG * SfU * SfU | | CCACAGGUU | SSSnRSS |
| WV- | fU * SfU * SfGn001RfU * SfG * SfUn001RmCfA * SmCmC * SfA * | 170 | UUGUGUCACCA | SSnRSSnROSOSSOO |
| 31560 | SmGmAfG * SfU * SfA * SfAn001RfC * SfA * SfG | | GAGUAACAG | SSSnRSS |
| WV- | fG * SfG * SfUn001RfA * SfA * SfGn001RfU * SfU * SmCfU * SmG | 171 | GGUAAGUUCUG | SSn RSSn RSSOSSSOS |
| 31561 | * SfU * SmCfC * SfA * SfA * SfGn001RfC * SfC * SfC | | UCCAAGCCC | SSnRSS |
| WV- | fG * SfU * SfAn001RfC * SfC * SfUn001RfC * SfC * SmAfA * SmC | 172 | GUACCUCCAAC | SSn RSSn RSSOSSSOS |
| 31562 | * SfA * SmUfC * SfA * SfA * SfGn001RfG * SfA * SfA | | AUCAAGGAA | SSnRSS |
| WV- | fC * SfA * SfAn001RfC * SfA * SfUn001RfC * SfA * SmAfG * SmG | 173 | CAACAUCAAGG | SSn RSSn RSSOSSSOS |
| 31563 | * SfA * SmAfG * SfA * SfU * SfGn001RfG * SfC * SfA | | AAGAUGGCA | SSnRSS |
| WV- | fA * SfU * SfGn001RfG * SfC * SfAn001RfU * SfU * SmUfC * SmU | 174 | AUGGCAUUUCU | SSn RSSn RSSOSSSOS |
| 31564 | * SfA * SmGfU * SfU * SfU * SfGn001RfG * SfA * SfG | | AGUUUGGAG | SSnRSS |
| WV- | fU * SfG * SfGn001RfC * SfA * SfUn001RfU * SfU * SmCfU * SmA | 175 | UGGCAUUUCUA | SSn RSSn RSSOSSSOS |
| 31565 | * SfG * SmUfU * SfU * SfG * SfGn001RfA * SfG * SfA | | GUUUGGAGA | SSnRSS |
| WV- | fG * SfG * SfCn001RfA * SfU * SfUn001RfU * SfC * SmUfA * SmG | 176 | GGCAUUUCUAG | SSn RSSn RSSOSSSOS |
| 31566 | * SfU * SmUfU * SfG * SfG * SfAn001RfG * SfA * SfU | | UUUGGAGAU | SSnRSS |
| WV- | fG * SfC * SfAn001RfU * SfU * SfUn001RfC * SfU * SmAfG * SmU | 177 | GCAUUUCUAGU | SSnRSSnRSSOSSSOS |
| 31567 | * SfU * SmUfG * SfG * SfA * SfGn001RfA * SfU * SfG | | UUGGAGAUG | SSnRSS |
| WV- | fU * SfG * SfGn001RfC * SfA * SfGn001RfU * SfU * SmUfC * SmC | 178 | UGGCAGUUUCC | SSn RSSn RSSOSSSOS |
| 31568 | * SfU * SmUfA * SfG * SfU * SfAn001RfA * SfC * SfC | | UUAGUAACC | SSnRSS |
| WV- | fC * SfA * SfGn001RfU * SfU * SfUn001RfC * SfC * SmUfU * SmA | 179 | CAGUUUCCUUA | SSn RSSn RSSOSSSOS |
| 31569 | * SfG * SmUfA * SfA * SfC * SfCn001RfA * SfC * SfA | | GUAACCACA | SSnRSS |
| WV- | fA * SfG * SfUn001RfU * SfU * SfCn001RfC * SfU * SmUfA * SmG | 180 | AGUUUCCUUAG | SSn RSSn RSSOSSSOS |
| 31570 | * SfU * SmAfA * SfC * SfC * SfAn001RfC * SfA * SfG | | UAACCACAG | SSnRSS |
| WV- | fU * SfU * SfCn001RfC * SfU * SfUn001RfA * SfG * SmUfA * SmA | 181 | UUCCUUAGUAA | SSn RSSn RSSOSSSOS |
| 31571 | * SfC * SmCfA * SfC * SfA * SfGn001RfG * SfU * SfU | | CCACAGGUU | SSnRSS |
| WV- | fU * SfU * SfGn001RfU * SfG * SfUn001RfC * SfA * SmCfC * SmA | 182 | UUGUGUCACCA | SSn RSSn RSSOSSSOS |
| 31572 | * SfG * SmAfG * SfU * SfA * SfAn001RfC * SfA * SfG | | GAGUAACAG | SSnRSS |
| WV- | fG * SfG * SfUn001RfA * SfA * SfGn001RmUfU * SfC * SmU * SfG | 183 | GGUAAGUUCUG | SSn RSSn ROSSSSOSS |
| 31573 | * SmUmC * SfC * SfA * SfA * SfGn001RfC * SfC * SfC | | UCCAAGCCC | SSnRSS |
| WV- | fG * SfU * SfAn001RfC * SfC * SfUn001RmCfC * SfA * SmA * SfC | 184 | GUACCUCCAAC | SSn RSSn ROSSSSOSS |
| 31574 | * SmAmU * SfC * SfA * SfA * SfGn001RfG * SfA * SfA | | AUCAAGGAA | SSnRSS |
| WV- | fC * SfA * SfAn001RfC * SfA * SfUn001RmCfA * SfA * SmG * SfG | 185 | CAACAUCAAGG | SSn RSSn ROSSSSOSS |
| 31575 | * SmAmA * SfG * SfA * SfU * SfGn001RfG * SfC * SfA | | AAGAUGGCA | SSnRSS |
| WV- | fA * SfU * SfGn001RfG * SfC * SfAn001RmUfU * SfU * SmC * SfU | 186 | AUGGCAUUUCU | SSn RSSn ROSSSSOSS |
| 31576 | * SmAmG * SfU * SfU * SfU * SfGn001RfG * SfA * SfG | | AGUUUGGAG | SSnRSS |
| WV- | fU * SfG * SfGn001RfC * SfA * SfUn001RmUfU * SfC * SmU * SfA | 187 | UGGCAUUUCUA | SSn RSSn ROSSSSOSS |
| 31577 | * SmGmU * SfU * SfU * SfG * SfGn001RfA * SfG * SfA | | GUUUGGAGA | SSnRSS |
| WV- | fG * SfG * SfCn001RfA * SfU * SfUn001RmUfC * SfU * SmA * SfG | 188 | GGCAUUUCUAG | SSn RSSn ROSSSSOSS |
| 31578 | * SmUmU * SfU * SfG * SfG * SfAn001RfG * SfA * SfU | | UUUGGAGAU | SSnRSS |
| WV- | fG * SfC * SfAn001RfU * SfU * SfUn001RmCfU * SfA * SmG * SfU | 189 | GCAUUUCUAGU | SSn RSSn ROSSSSOSS |
| 31579 | * SmUmU * SfG * SfG * SfA * SfGn001RfA * SfU * SfG | | UUGGAGAUG | SSnRSS |
| WV- | fU * SfG * SfGn001RfC * SfA * SfGn001RmUfU * SfU * SmC * SfC | 190 | UGGCAGUUUCC | SSn RSSn ROSSSSOSS |
| 31580 | * SmUmU * SfA * SfG * SfU * SfAn001RfA * SfC * SfC | | UUAGUAACC | SSnRSS |
| WV- | fC * SfA * SfGn001RfU * SfU * SfUn001RmCfC * SfU * SmU * SfA | 191 | CAGUUUCCUUA | SSn RSSn ROSSSSOSS |
| 31581 | * SmGmU * SfA * SfA * SfC * SfCn001RfA * SfC * SfA | | GUAACCACA | SSnRSS |
| WV- | fA * SfG * SfUn001RfU * SfU * SfCn001RmCfU * SfU * SmA * SfG | 192 | AGUUUCCUUAG | SSn RSSn ROSSSSOSS |
| 31582 | * SmUmA * SfA * SfC * SfC * SfAn001RfC * SfA * SfG | | UAACCACAG | SSnRSS |
| WV- | fU * SfU * SfCn001RfC * SfU * SfUn001RmAfG * SfU * SmA * SfA | 193 | UUCCUUAGUAA | SSn RSSn ROSSSSOSS |
| 31583 | * SmCmC * SfA * SfC * SfA * SfGn001RfG * SfU * SfU | | CCACAGGUU | SSnRSS |
| WV- | fU * SfU * SfGn001RfU * SfG * SfUn001RmCfA * SfC * SmC * SfA | 194 | UUGUGUCACCA | SSn RSSn ROSSSSOSS |
| 31584 | * SmGmA * SfG * SfU * SfA * SfAn001RfC * SfA * SfG | | GAGUAACAG | SSnRSS |
| WV- | fU * SfC * SfAn001RfA * SfG * SfGn001RmAfA * SmGfA * SmUfG | 195 | UCAAGGAAGAU | SSnRSSnROSOSOSO |
| 31585 | * SmGfC * SfA * SfU * SfUn001RfU * SfC * SfU | | GGCAUUUCU | SSSnRSS |
| WV- | fU * SfC * SfAn001RfA * SfG * SfGn001RmAfA * SmGmA * SfU * | 196 | UCAAGGAAGAU | SSnRSSnROSOSSOO |
| 31586 | SmGmGfC * SfA * SfU * SfUn001RfU * SfC * SfU | | GGCAUUUCU | SSSnRSS |
| WV- | fU * SfC * SfAn001RfA * SfG * SfGn001RfA * SfA * SmGfA * SmU | 197 | UCAAGGAAGAU | SSnRSSnRSSOSSSOS |
| 31587 | * SfG * SmGfC * SfA * SfU * SfUn001RfU * SfC * SfU | | GGCAUUUCU | SSnRSS |
| WV- | fU * SfC * SfAn001RfA * SfG * SfGn001RmAfA * SfG * SmA * SfU | 198 | UCAAGGAAGAU | SSnRSSnROSSSSOSS |
| 31588 | * SmGmG * SfC * SfA * SfU * SfUn001RfU * SfC * SfU | | GGCAUUUCU | SSnRSS |
| WV- | fC * SfU * SfUn001fC * SfU * SfGn001fC * SfC * SmA * SfA * SmC | 199 | CUUCUGCCAAC | SSnX SSnX SSSSS |
| 19886 | * SfU * SmU * SfU * SfU * SfA * SfUn001fC * SfA * SfU | | UUUUAUCAU | SSSSS nX SS |
| WV- | fU * SfU * SfCn001fU * SfG * SfCn001fC * SfA * SmA * SfC * SmU | 200 | UUCUGCCAACU | SSnX SSnX SSSSS |
| 19887 | * SfU * SmU * SfU * SfA * SfU * SfCn001fA * SfU * SfU | | UUUAUCAUU | SSSSS nX SS |
| WV- | fU * SfC * SfUn001fG * SfC * SfCn001fA * SfA * SmC * SfU * SmU | 201 | UCUGCCAACUU | SSnX SSnX SSSSS |
| 19888 | * SfU * SmU * SfA * SfU * SfC * SfAn001fU * SfU * SfU | | UUAUCAUUU | SSSSS nX SS |
| WV- | fC * SfU * SfGn001fC * SfC * SfAn001fA * SfC * SmU * SfU * SmU | 202 | CUGCCAACUUU | SSnX SSnX SSSSS |
| 19889 | * SfU * SmA * SfU * SfC * SfA * SfUn001fU * SfU * SfU | | UAUCAUUUU | SSSSS nX SS |
| WV- | fU * SfG * SfCn001fC * SfA * SfAn001fC * SfU * SmU * SfU * SmU | 203 | UGCCAACUUUU | SSnX SSnX SSSSS |
| 19890 | * SfA * SmU * SfC * SfA * SfU * SfUn001fU * SfU * SfU | | AUCAUUUUU | SSSSS nX SS |
| WV- | fG * SfC * SfCn001fA * SfA * SfCn001fU * SfU * SmU * SfU * SmA | 204 | GCCAACUUUUA | SSnX SSnX SSSSS |
| 19891 | * SfU * SmC * SfA * SfU * SfU * SfUn001fU * SfU * SfU | | UCAUUUUUU | SSSSS nX SS |
| WV- | fC * SfC * SfAn001fA * SfC * SfUn001fU * SfU * SmU * SfA * SmU | 205 | CCAACUUUUAU | SSnX SSnX SSSSS |
| 19892 | * SfC * SmA * SfU * SfU * SfU * SfUn001fU * SfU * SfC | | CAUUUUUUC | SSSSS nX SS |
| WV- | fC * SfA * SfAn001fC * SfU * SfUn001fU * SfU * SmA * SfU * SmC | 206 | CAACUUUUAUC | SSnX SSnX SSSSS |
| 19893 | * SfA * SmU * SfU * SfU * SfU * SfUn001fU * SfC * SfU | | AUUUUUUCU | SSSSS nX SS |
| WV- | fA * SfA * SfCn001fU * SfU * SfUn001fU * SfA * SmU * SfC * SmA | 207 | AACUUUUAUCA | SSnX SSnX SSSSS |
| 19894 | * SfU * SmU * SfU * SfU * SfU * SfUn001fC * SfU * SfC | | UUUUUUCUC | SSSSS nX SS |
| WV- | fA * SfC * SfUn001fU * SfU * SfUn001fA * SfU * SmC * SfA * SmU | 208 | ACUUUUAUCAU | SSnX SSnX SSSSS |
| 19895 | * SfU * SmU * SfU * SfU * SfU * SfCn001fU * SfC * SfA | | UUUUUCUCA | SSSSS nX SS |
| WV- | fC * SfU * SfUn001fU * SfU * SfAn001fU * SfC * SmA * SfU * SmU | 209 | CUUUUAUCAUU | SSnX SSnX SSSSS |
| 19896 | * SfU * SmU * SfU * SfU * SfC * SfUn001fC * SfA * SfU | | UUUUCUCAU | SSSSS nX SS |
| WV- | fU * SfU * SfUn001fU * SfA * SfUn001fC * SfA * SmU * SfU * SmU | 210 | UUUUAUCAUUU | SSnX SSnX SSSSS |
| 19897 | * SfU * SmU * SfU * SfC * SfU * SfCn001fA * SfU * SfA | | UUUCUCAUA | SSSSS nX SS |
| WV- | fU * SfU * SfUn001fA * SfU * SfCn001fA * SfU * SmU * SfU * SmU | 211 | UUUAUCAUUUU | SSnX SSnX SSSSS |
| 19898 | * SfU * SmU * SfC * SfU * SfC * SfAn001fU * SfA * SfC | | UUCUCAUAC | SSSSS nX SS |
| WV- | fU * SfU * SfAn001fU * SfC * SfAn001fU * SfU * SmU * SfU * SmU | 212 | UUAUCAUUUUU | SSnX SSnX SSSSS |
| 19899 | * SfU * SmC * SfU * SfC * SfA * SfUn001fA * SfC * SfC | | UCUCAUACC | SSSSS nX SS |
| WV- | fU * SfA * SfUn001fC * SfA * SfUn001fU * SfU * SmU * SfU * SmU | 213 | UAUCAUUUUUU | SSnX SSnX SSSSS |
| 19900 | * SfC * SmU * SfC * SfA * SfU * SfAn001fC * SfC * SfU | | CUCAUACCU | SSSSS nX SS |
| WV- | fA * SfU * SfCn001fA * SfU * SfUn001fU * SfU * SmU * SfU * SmC | 214 | AUCAUUUUUUC | SSnX SSnX SSSSS |
| 19901 | * SfU * SmC * SfA * SfU * SfA * SfCn001fC * SfU * SfU | | UCAUACCUU | SSSSS nX SS |
| WV- | fU * SfC * SfAn001fU * SfU * SfUn001fU * SfU * SmU * SfC * SmU | 215 | UCAUUUUUUCU | SSnX SSnX SSSSS |
| 19902 | * SfC * SmA * SfU * SfA * SfC * SfCn001fU * SfU * SfC | | CAUACCUUC | SSSSS nX SS |
| WV- | fC * SfA * SfUn001fU * SfU * SfUn001fU * SfU * SmC * SfU * SmC | 216 | CAUUUUUUCUC | SSnX SSnX SSSSS |
| 19903 | * SfA * SmU * SfA * SfC * SfC * SfUn001fU * SfC * SfU | | AUACCUUCU | SSSSS nX SS |
| WV- | fA * SfU * SfUn001fU * SfU * SfUn001fU * SfC * SmU * SfC * SmA | 217 | AUUUUUUCUCA | SSnX SSnX SSSSS |
| 19904 | * SfU * SmA * SfC * SfC * SfU * SfUn001fC * SfU * SfG | | UACCUUCUG | SSSSS nX SS |
| WV- | fU * SfU * SfUn001fU * SfU * SfUn001fC * SfU * SmC * SfA * SmU | 218 | UUUUUUCUCAU | SSnX SSnX SSSSS |
| 19905 | * SfA * SmC * SfC * SfU * SfU * SfCn001fU * SfG * SfC | | ACCUUCUGC | SSSSS nX SS |
| WV- | fU * SfU * SfUn001fU * SfU * SfCn001fU * SfC * SmA * SfU * SmA | 219 | UUUUUCUCAUA | SSnX SSnX SSSSS |
| 19906 | * SfC * SmC * SfU * SfU * SfC * SfUn001fG * SfC * SfU | | CCUUCUGCU | SSSSS nX SS |
| WV- | fU * SfU * SfUn001fU * SfC * SfUn001fC * SfA * SmU * SfA * SmC | 220 | UUUUCUCAUAC | SSnX SSnX SSSSS |
| 19907 | * SfC * SmU * SfU * SfC * SfU * SfGn001fC * SfU * SfU | | CUUCUGCUU | SSSSS nX SS |
| WV- | fU * SfU * SfUn001fC * SfU * SfCn001fA * SfU * SmA * SfC * SmC | 221 | UUUCUCAUACC | SSnX SSnX SSSSS |
| 19908 | * SfU * SmU * SfC * SfU * SfG * SfCn001fU * SfU * SfG | | UUCUGCUUG | SSSSS nX SS |
| WV- | fU * SfU * SfCn001fU * SfC * SfAn001fU * SfA * SmC * SfC * SmU | 222 | UUCUCAUACCU | SSnX SSnX SSSSS |
| 19909 | * SfU * SmC * SfU * SfG * SfC * SfUn001fU * SfG * SfA | | UCUGCUUGA | SSSSS nX SS |
| WV- | fU * SfC * SfUn001fC * SfA * SfUn001fA * SfC * SmC * SfU * SmU | 223 | UCUCAUACCUU | SSnX SSnX SSSSS |
| 19910 | * SfC * SmU * SfG * SfC * SfU * SfUn001fG * SfA * SfU | | CUGCUUGAU | SSSSS nX SS |
| WV- | fC * SfU * SfCn001fA * SfU * SfAn001fC * SfC * SmU * SfU * SmC | 224 | CUCAUACCUUC | SSnX SSnX SSSSS |
| 19911 | * SfU * SmG * SfC * SfU * SfU * SfGn001fA * SfU * SfG | | UGCUUGAUG | SSSSS nX SS |
| WV- | fU * SfC * SfAn001fU * SfA * SfCn001fC * SfU * SmU * SfC * SmU | 225 | UCAUACCUUCU | SSnX SSnX SSSSS |
| 19912 | * SfG * SmC * SfU * SfU * SfG * SfAn001fU * SfG * SfA | | GCUUGAUGA | SSSSS nX SS |
| WV- | fC * SfA * SfUn001fA * SfC * SfCn001fU * SfU * SmC * SfU * SmG | 226 | CAUACCUUCUG | SSnX SSnX SSSSS |
| 19913 | * SfC * SmU * SfU * SfG * SfA * SfUn001fG * SfA * SfU | | CUUGAUGAU | SSSSS nX SS |
| WV- | fA * SfU * SfAn001fC * SfC * SfUn001fU * SfC * SmU * SfG * SmC | 227 | AUACCUUCUGC | SSnX SSnX SSSSS |
| 19914 | * SfU * SmU * SfG * SfA * SfU * SfGn001fA * SfU * SfC | | UUGAUGAUC | SSSSS nX SS |
| WV- | fU * SfA * SfCn001fC * SfU * SfUn001fC * SfU * SmG * SfC * SmU | 228 | UACCUUCUGCU | SSnX SSnX SSSSS |
| 19915 | * SfU * SmG * SfA * SfU * SfG * SfAn001fU * SfC * SfA | | UGAUGAUCA | SSSSS nX SS |
| WV- | fA * SfC * SfCn001fU * SfU * SfCn001fU * SfG * SmC * SfU * SmU | 229 | ACCUUCUGCUU | SSnX SSnX SSSSS |
| 19916 | * SfG * SmA * SfU * SfG * SfA * SfUn001fC * SfA * SfU | | GAUGAUCAU | SSSSS nX SS |
| WV- | fC * SfC * SfUn001fU * SfC * SfUn001fG * SfC * SmU * SfU * SmG | 230 | CCUUCUGCUUG | SSnX SSnX SSSSS |
| 19917 | * SfA * SmU * SfG * SfA * SfU * SfCn001fA * SfU * SfC | | AUGAUCAUC | SSSSS nX SS |
| WV- | fC * SfU * SfUn001fC * SfU * SfGn001fC * SfU * SmU * SfG * SmA | 231 | CUUCUGCUUGA | SSnX SSnX SSSSS |
| 19918 | * SfU * SmG * SfA * SfU * SfC * SfAn001fU * SfC * SfU | | UGAUCAUCU | SSSSS nX SS |
| WV- | fU * SfU * SfCn001fU * SfG * SfCn001fU * SfU * SmG * SfA * SmU | 232 | UUCUGCUUGAU | SSnX SSnX SSSSS |
| 19919 | * SfG * SmA * SfU * SfC * SfA * SfUn001fC * SfU * SfC | | GAUCAUCUC | SSSSS nX SS |
| WV- | fU * SfC * SfUn001fG * SfC * SfUn001fU * SfG * SmA * SfU * SmG | 233 | UCUGCUUGAUG | SSnX SSnX SSSSS |
| 19920 | * SfA * SmU * SfC * SfA * SfU * SfCn001fU * SfC * SfG | | AUCAUCUCG | SSSSS nX SS |
| WV- | fC * SfU * SfGn001fC * SfU * SfUn001fG * SfA * SmU * SfG * SmA | 234 | CUGCUUGAUGA | SSnX SSnX SSSSS |
| 19921 | * SfU * SmC * SfA * SfU * SfC * SfUn001fC * SfG * SfU | | UCAUCUCGU | SSSSS nX SS |
| WV- | fU * SfG * SfCn001fU * SfU * SfGn001fA * SfU * SmG * SfA * SmU | 235 | UGCUUGAUGAU | SSnX SSnX SSSSS |
| 19922 | * SfC * SmA * SfU * SfC * SfU * SfCn001fG * SfU * SfU | | CAUCUCGUU | SSSSS nX SS |
| WV- | fG * SfC * SfUn001fU * SfG * SfAn001fU * SfG * SmA * SfU * SmC | 236 | GCUUGAUGAUC | SSnX SSnX SSSSS |
| 19923 | * SfA * SmU * SfC * SfU * SfC * SfGn001fU * SfU * SfG | | AUCUCGUUG | SSSSS nX SS |
| WV- | fC * SfU * SfUn001fG * SfA * SfUn001fG * SfA * SmU * SfC * SmA | 237 | CUUGAUGAUCA | SSnX SSnX SSSSS |
| 19924 | * SfU * SmC * SfU * SfC * SfG * SfUn001fU * SfG * SfA | | UCUCGUUGA | SSSSS nX SS |
| WV- | fU * SfU * SfGn001fA * SfU * SfGn001fA * SfU * SmC * SfA * SmU | 238 | UUGAUGAUCAU | SSnX SSnX SSSSS |
| 19925 | * SfC * SmU * SfC * SfG * SfU * SfUn001fG * SfA * SfU | | CUCGUUGAU | SSSSS nX SS |
| WV- | fU * SfG * SfAn001fU * SfG * SfAn001fU * SfC * SmA * SfU * SmC | 239 | UGAUGAUCAUC | SSnX SSnX SSSSS |
| 19926 | * SfU * SmC * SfG * SfU * SfU * SfGn001fA * SfU * SfA | | UCGUUGAUA | SSSSS nX SS |
| WV- | fG * SfA * SfUn001fG * SfA * SfUn001fC * SfA * SmU * SfC * SmU | 240 | GAUGAUCAUCU | SSnX SSnX SSSSS |
| 19927 | * SfC * SmG * SfU * SfU * SfG * SfAn001fU * SfA * SfU | | CGUUGAUAU | SSSSS nX SS |
| WV- | fA * SfU * SfGn001fA * SfU * SfCn001fA * SfU * SmC * SfU * SmC | 241 | AUGAUCAUCUC | SSnX SSnX SSSSS |
| 19928 | * SfG * SmU * SfU * SfG * SfA * SfUn001fA * SfU * SfC | | GUUGAUAUC | SSSSS nX SS |
| WV- | fU * SfG * SfAn001fU * SfC * SfAn001fU * SfC * SmU * SfC * SmG | 242 | UGAUCAUCUCG | SSnX SSnX SSSSS |
| 19929 | * SfU * SmU * SfG * SfA * SfU * SfAn001fU * SfC * SfC | | UUGAUAUCC | SSSSS nX SS |
| WV- | fG * SfA * SfUn001fC * SfA * SfUn001fC * SfU * SmC * SfG * SmU | 243 | GAUCAUCUCGU | SSnX SSnX SSSSS |
| 19930 | * SfU * SmG * SfA * SfU * SfA * SfUn001fC * SfC * SfU | | UGAUAUCCU | SSSSS nX SS |
| WV- | fA * SfU * SfCn001fA * SfU * SfCn001fU * SfC * SmG * SfU * SmU | 244 | AUCAUCUCGUU | SSnX SSnX SSSSS |
| 19931 | * SfG * SmA * SfU * SfA * SfU * SfCn001fC * SfU * SfC | | GAUAUCCUC | SSSSS nX SS |
| WV- | fU * SfC * SfAn001fU * SfC * SfUn001fC * SfG * SmU * SfU * SmG | 245 | UCAUCUCGUUG | SSnX SSnX SSSSS |
| 19932 | * SfA * SmU * SfA * SfU * SfC * SfCn001fU * SfC * SfA | | AUAUCCUCA | SSSSS nX SS |
| WV- | fC * SfA * SfUn001fC * SfU * SfC001fG * SfU * SmU * SfG * SmA | 246 | CAUCUCGUUGA | SSnX SSnX SSSSS |
| 19933 | * SfU * SmA * SfU * SfC * SfC * SfUn001fC * SfA * SfA | | UAUCCUCAA | SSSSS nX SS |
| WV- | fA * SfU * SfCn001fU * SfC * SfGn001fU * SfU * SmG * SfA * SmU | 247 | AUCUCGUUGAU | SSnX SSnX SSSSS |
| 19934 | * SfA * SmU * SfC * SfC * SfU * SfCn001fA * SfA * SfG | | AUCCUCAAG | SSSSS nX SS |
| WV- | fU * SfC * SfUn001fC * SfG * SfUn001fU * SfG * SmA * SfU * SmA | 248 | UCUCGUUGAUA | SSnX SSnX SSSSS |
| 19935 | * SfU * SmC * SfC * SfU * SfC * SfAn001fA * SfG * SfG | | UCCUCAAGG | SSSSS nX SS |
| WV- | fC * SfU * SfCn001fG * SfU * SfUn001fG * SfA * SmU * SfA * SmU | 249 | CUCGUUGAUAU | SSnX SSnX SSSSS |
| 19936 | * SfC * SmC * SfU * SfC * SfA * SfAn001fG * SfG * SfU | | CCUCAAGGU | SSSSS nX SS |
| WV- | fU * SfC * SfGn001fU * SfU * SfGn001fA * SfU * SmA * SfU * SmC | 250 | UCGUUGAUAUC | SSnX SSnX SSSSS |
| 19937 | * SfC * SmU * SfC * SfA * SfA * SfGn001fG * SfU * SfC | | CUCAAGGUC | SSSSS nX SS |
| WV- | fC * SfG * SfUn001fU * SfG * SfAn001fU * SfA * SmU * SfC * SmC | 251 | CGUUGAUAUCC | SSnX SSnX SSSSS |
| 19938 | * SfU * SmC * SfA * SfA * SfG * SfGn001fU * SfC * SfA | | UCAAGGUCA | SSSSS nX SS |
| WV- | fG * SfU * SfUn001fG * SfA * SfUn001fA * SfU * SmC * SfC * SmU | 252 | GUUGAUAUCCU | SSnX SSnX SSSSS |
| 19939 | * SfC * SmA * SfA * SfG * SfG * SfUn001fC * SfA * SfC | | CAAGGUCAC | SSSSS nX SS |
| WV- | fU * SfU * SfGn001fA * SfU * SfAn001fU * SfC * SmC * SfU * SmC | 253 | UUGAUAUCCUC | SSnX SSnX SSSSS |
| 19940 | * SfA * SmA * SfG * SfG * SfU * SfCn001fA * SfC * SfC | | AAGGUCACC | SSSSS nX SS |
| WV- | fU * SfG * SfAn001fU * SfA * SfUn001fC * SfC * SmU * SfC * SmA | 254 | UGAUAUCCUCA | SSnX SSnX SSSSS |
| 19941 | * SfA * SmG * SfG * SfU * SfC * SfAn001fC * SfC * SfC | | AGGUCACCC | SSSSS nX SS |
| WV- | fG * SfA * SfUn001fA * SfU * SfCn001fC * SfU * SmC * SfA * SmA | 255 | GAUAUCCUCAA | SSnX SSnX SSSSS |
| 19942 | * SfG * SmG * SfU * SfC * SfA * SfCn001fC * SfC * SfA | | GGUCACCCA | SSSSS nX SS |
| WV- | fA * SfU * SfAn001fU * SfC * SfCn001fU * SfC * SmA * SfA * SmG | 256 | AUAUCCUCAAG | SSnX SSnX SSSSS |
| 19943 | * SfG * SmU * SfC * SfA * SfC * SfCn001fC * SfA * SfC | | GUCACCCAC | SSSSS nX SS |
| WV- | fU * SfA * SfUn001fC * SfC * SfUn001fC * SfA * SmA * SfG * SmG | 257 | UAUCCUCAAGG | SSnX SSnX SSSSS |
| 19944 | * SfU * SmC * SfA * SfC * SfC * SfCn001fA * SfC * SfC | | UCACCCACC | SSSSS nX SS |
| WV- | fA * SfU * SfCn001fC * SfU * SfCn001fA * SfA * SmG * SfG * SmU | 258 | AUCCUCAAGGU | SSnX SSnX SSSSS |
| 19945 | * SfC * SmA * SfC * SfC * SfC * SfAn001fC * SfC * SfA | | CACCCACCA | SSSSS nX SS |
| WV- | fU * SfC * SfCn001fU * SfC * SfAn001fA * SfG * SmG * SfU * SmC | 259 | UCCUCAAGGUC | SSnX SSnX SSSSS |
| 19946 | * SfA * SmC * SfC * SfC * SfA * SfCn001fC * SfA * SfU | | ACCCACCAU | SSSSS nX SS |
| WV- | fC * SfC * SfUn001fC * SfA * SfAn001fG * SfG * SmU * SfC * SmA | 260 | CCUCAAGGUCA | SSnX SSnX SSSSS |
| 19947 | * SfC * SmC * SfC * SfA * SfC * SfCn001fA * SfU * SfC | | CCCACCAUC | SSSSS nX SS |
| WV- | fC * SfU * SfCn001fA * SfA * SfGn001fG * SfU * SmC * SfA * SmC | 261 | CUCAAGGUCAC | SSnX SSnX SSSSS |
| 19948 | * SfC * SmC * SfA * SfC * SfC * SfAn001fU * SfC * SfA | | CCACCAUCA | SSSSS nX SS |
| WV- | fU * SfC * SfAn001fA * SfG * SfGn001fU * SfC * SmA * SfC * SmC | 262 | UCAAGGUCACC | SSnX SSnX SSSSS |
| 19949 | * SfC * SmA * SfC * SfC * SfA * SfUn001fC * SfA * SfC | | CACCAUCAC | SSSSS nX SS |
| WV- | fC * SfA * SfAn001fG * SfG * SfUn001fC * SfA * SmC * SfC * SmC | 263 | CAAGGUCACCC | SSnX SSnX SSSSS |
| 19950 | * SfA * SmC * SfC * SfA * SfU * SfCn001fA * SfC * SfC | | ACCAUCACC | SSSSS nX SS |
| WV- | fA * SfA * SfGn001fG * SfU * SfCn001fA * SfC * SmC * SfC * SmA | 264 | AAGGUCACCCA | SSnX SSnX SSSSS |
| 19951 | * SfC * SmC * SfA * SfU * SfC * SfAn001fC * SfC * SfC | | CCAUCACCC | SSSSS nX SS |
| WV- | fA * SfG * SfGn001fU * SfC * SfAn001fC * SfC * SmC * SfA * SmC | 265 | AGGUCACCCAC | SSnX SSnX SSSSS |
| 19952 | * SfC * SmA * SfU * SfC * SfA * SfCn001fC * SfC * SfU | | CAUCACCCU | SSSSS nX SS |
| WV- | fG * SfG * SfUn001fC * SfA * SfCn001fC * SfC * SmA * SfC * SmC | 266 | GGUCACCCACC | SSnX SSnX SSSSS |
| 19953 | * SfA * SmU * SfC * SfA * SfC * SfCn001fC * SfU * SfC | | AUCACCCUC | SSSSS nX SS |
| WV- | fA * SfC * SfCn001fC * SfA * SfCn001fC * SfA * SmU * SfC * SmA | 267 | ACCCACCAUCA | SSnX SSnX SSSSS |
| 19957 | * SfC * SmC * SfC * SfU * SfC * SfUn001fG * SfU * SfG | | CCCUCUGUG | SSSSS nX SS |
| WV- | fC * SfC * SfCn001fA * SfC * SfCn001fA * SfU * SmC * SfA * SmC | 268 | CCCACCAUCAC | SSnX SSnX SSSSS |
| 19958 | * SfC * SmC * SfU * SfC * SfU * SfGn001fU * SfG * SfA | | CCUCUGUGA | SSSSS nX SS |
| WV- | fC * SfC * SfAn001fC * SfC * SfAn001fU * SfC * SmA * SfC * SmC | 269 | CCACCAUCACC | SSnX SSnX SSSSS |
| 19959 | * SfC * SmU * SfC * SfU * SfG * SfUn001fG * SfA * SfU | | CUCUGUGAU | SSSSS nX SS |
| WV- | fC * SfA * SfCn001fC * SfA * SfUn001fC * SfA * SmC * SfC * SmC | 270 | CACCAUCACCC | SSnX SSnX SSSSS |
| 19960 | * SfU * SmC * SfU * SfG * SfU * SfGn001fA * SfU * SfU | | UCUGUGAUU | SSSSS nX SS |
| WV- | fA * SfC * SfCn001fA * SfU * SfCn001fA * SfC * SmC * SfC * SmU | 271 | ACCAUCACCCU | SSnX SSnX SSSSS |
| 19961 | * SfC * SmU * SfG * SfU * SfG * SfAn001fU * SfU * SfU | | CUGUGAUUU | SSSSS nX SS |
| WV- | fC * SfC * SfAn001fU * SfC * SfAn001fC * SfC * SmC * SfU * SmC | 272 | CCAUCACCCUC | SSnX SSnX SSSSS |
| 19962 | * SfU * SmG * SfU * SfG * SfA * SfUn001fU * SfU * SfU | | UGUGAUUUU | SSSSS nX SS |
| WV- | fC * SfA * SfUn001fC * SfA * SfCn001fC * SfC * SmU * SfC * SmU | 273 | CAUCACCCUCU | SSnX SSnX SSSSS |
| 19963 | * SfG * SmU * SfG * SfA * SfU * SfUn001fU * SfU * SfA | | GUGAUUUUA | SSSSS nX SS |
| WV- | fA * SfU * SfCn001fA * SfC * SfCn001fC * SfU * SmC * SfU * SmG | 274 | AUCACCCUCUG | SSnX SSnX SSSSS |
| 19964 | * SfU * SmG * SfA * SfU * SfU * SfUn001fU * SfA * SfU | | UGAUUUUAU | SSSSS nX SS |
| WV- | fU * SfC * SfAn001fC * SfC * SfCn001fU * SfC * SmU * SfG * SmU | 275 | UCACCCUCUGU | SSnX SSnX SSSSS |
| 19965 | * SfG * SmA * SfU * SfU * SfU * SfUn001fA * SfU * SfA | | GAUUUUAUA | SSSSS nX SS |
| WV- | fC * SfA * SfCn001fC * SfC * SfUn001fC * SfU * SmG * SfU * SmG | 276 | CACCCUCUGUG | SSnX SSnX SSSSS |
| 19966 | * SfA * SmU * SfU * SfU * SfU * SfAn001fU * SfA * SfA | | AUUUUAUAA | SSSSS nX SS |
| WV- | fA * SfC * SfCn001fC * SfU * SfCn001fU * SfG * SmU * SfG * SmA | 277 | ACCCUCUGUGA | SSnX SSnX SSSSS |
| 19967 | * SfU * SmU * SfU * SfU * SfA * SfUn001fA * SfA * SfC | | UUUUAUAAC | SSSSS nX SS |
| WV- | fC * SfC * SfCn001fU * SfC * SfUn001fG * SfU * SmG * SfA * SmU | 278 | CCCUCUGUGAU | SSnX SSnX SSSSS |
| 19968 | * SfU * SmU * SfU * SfA * SfU * SfAn001fA * SfC * SfU | | UUUAUAACU | SSSSS nX SS |
| WV- | fC * SfC * SfUn001fC * SfU * SfGn001fU * SfG * SmA * SfU * SmU | 279 | CCUCUGUGAUU | SSnX SSnX SSSSS |
| 19969 | * SfU * SmU * SfA * SfU * SfA * SfAn001fC * SfU * SfU | | UUAUAACUU | SSSSS nX SS |
| WV- | fC * SfU * SfCn001fU * SfG * SfUn001fG * SfA * SmU * SfU * SmU | 280 | CUCUGUGAUUU | SSnX SSnX SSSSS |
| 19970 | * SfU * SmA * SfU * SfA * SfA * SfCn001fU * SfU * SfG | | UAUAACUUG | SSSSS nX SS |
| WV- | fU * SfC * SfUn001fG * SfU * SfGn001fA * SfU * SmU * SfU * SmU | 281 | UCUGUGAUUUU | SSnX SSnX SSSSS |
| 19971 | * SfA * SmU * SfA * SfA * SfC * SfUn001fU * SfG * SfA | | AUAACUUGA | SSSSS nX SS |
| WV- | fC * SfU * SfGn001fU * SfG * SfAn001fU * SfU * SmU * SfU * SmA | 282 | CUGUGAUUUUA | SSnX SSnX SSSSS |
| 19972 | * SfU * SmA * SfA * SfC * SfU * SfUn001fG * SfA * SfU | | UAACUUGAU | SSSSS nX SS |
| WV- | fU * SfG * SfUn001fG * SfA * SfUn001fU * SfU * SmU * SfA * SmU | 283 | UGUGAUUUUAU | SSnX SSnX SSSSS |
| 19973 | * SfA * SmA * SfC * SfU * SfU * SfGn001fA * SfU * SfC | | AACUUGAUC | SSSSS nX SS |
| WV- | fG * SfU * SfGn001fA * SfU * SfUn001fU * SfU * SmA * SfU * SmA | 284 | GUGAUUUUAUA | SSnX SSnX SSSSS |
| 19974 | * SfA * SmC * SfU * SfU * SfG * SfAn001fU * SfC * SfA | | ACUUGAUCA | SSSSS nX SS |
| WV- | fU * SfG * SfAn001fU * SfU * SfUn001fU * SfA * SmU * SfA * SmA | 285 | UGAUUUUAUAA | SSnX SSnX SSSSS |
| 19975 | * SfC * SmU * SfU * SfG * SfA * SfUn001fC * SfA * SfA | | CUUGAUCAA | SSSSS nX SS |
| WV- | fG * SfA * SfUn001fU * SfU * SfUn001fA * SfU * SmA * SfA * SmC | 286 | GAUUUUAUAAC | SSnX SSnX SSSSS |
| 19976 | * SfU * SmU * SfG * SfA * SfU * SfCn001fA * SfA * SfG | | UUGAUCAAG | SSSSS nX SS |
| WV- | fA * SfU * SfUn001fU * SfU * SfAn001fU * SfA * SmA * SfC * SmU | 287 | AUUUUAUAACU | SSnX SSnX SSSSS |
| 19977 | * SfU * SmG * SfA * SfU * SfC * SfAn001fA * SfG * SfC | | UGAUCAAGC | SSSSS nX SS |
| WV- | fU * SfU * SfUn001fU * SfA * SfUn001fA * SfA * SmC * SfU * SmU | 288 | UUUUAUAACUU | SSnX SSnX SSSSS |
| 19978 | * SfG * SmA * SfU * SfC * SfA * SfAn001fG * SfC * SfA | | GAUCAAGCA | SSSSS nX SS |
| WV- | fU * SfU * SfUn001fA * SfU * SfAn001fA * SfC * SmU * SfU * SmG | 289 | UUUAUAACUUG | SSnX SSnX SSSSS |
| 19979 | * SfA * SmU * SfC * SfA * SfA * SfGn001fC * SfA * SfG | | AUCAAGCAG | SSSSS nX SS |
| WV- | fU * SfU * SfAn001fU * SfA * SfAn001fC * SfU * SmU * SfG * SmA | 290 | UUAUAACUUGA | SSnX SSnX SSSSS |
| 19980 | * SfU * SmC * SfA * SfA * SfG * SfCn001fA * SfG * SfA | | UCAAGCAGA | SSSSS nX SS |
| WV- | fU * SfA * SfUn001fA * SfA * SfCn001fU * SfU * SmG * SfA * SmU | 291 | UAUAACUUGAU | SSnX SSnX SSSSS |
| 19981 | * SfC * SmA * SfA * SfG * SfC * SfAn001fG * SfA * SfG | | CAAGCAGAG | SSSSS nX SS |
| WV- | fA * SfU * SfAn001fA * SfC * SfUn001fU * SfG * SmA * SfU * SmC | 292 | AUAACUUGAUC | SSnX SSnX SSSSS |
| 19982 | * SfA * SmA * SfG * SfC * SfA * SfGn001fA * SfG * SfA | | AAGCAGAGA | SSSSS nX SS |
| WV- | fU * SfA * SfAn001fC * SfU * SfUn001fG * SfA * SmU * SfC * SmA | 293 | UAACUUGAUCA | SSnX SSnX SSSSS |
| 19983 | * SfA * SmG * SfC * SfA * SfG * SfAn001fG * SfA * SfA | | AGCAGAGAA | SSSSS nX SS |
| WV- | fA * SfA * SfCn001fU * SfU * SfGn001fA * SfU * SmC * SfA * SmA | 294 | AACUUGAUCAA | SSnX SSnX SSSSS |
| 19984 | * SfG * SmC * SfA * SfG * SfA * SfGn001fA * SfA * SfA | | GCAGAGAAA | SSSSS nX SS |
| WV- | fA * SfC * SfUn001fU * SfG * SfAn001fU * SfC * SmA * SfA * SmG | 295 | ACUUGAUCAAG | SSnX SSnX SSSSS |
| 19985 | * SfC * SmA * SfG * SfA * SfG * SfAn001fA * SfA * SfG | | CAGAGAAAG | SSSSS nX SS |
| WV- | fC * SfU * SfUn001fG * SfA * SfUn001fC * SfA * SmA * SfG * SmC | 296 | CUUGAUCAAGC | SSnX SSnX SSSSS |
| 19986 | * SfA * SmG * SfA * SfG * SfA * SfAn001fA * SfG * SfC | | AGAGAAAGC | SSSSS nX SS |
| WV- | fU * SfU * SfGn001fA * SfU * SfCn001fA * SfA * SmG * SfC * SmA | 297 | UUGAUCAAGCA | SSnX SSnX SSSSS |
| 19987 | * SfG * SmA * SfG * SfA * SfA * SfAn001fG * SfC * SfC | | GAGAAAGCC | SSSSS nX SS |
| WV- | fU * SfG * SfAn001fU * SfC * SfAn001fA * SfG * SmC * SfA * SmG | 298 | UGAUCAAGCAG | SSnX SSnX SSSSS |
| 19988 | * SfA * SmG * SfA * SfA * SfA * SfGn001fC * SfC * SfA | | AGAAAGCCA | SSSSS nX SS |
| WV- | fG * SfA * SfUn001fC * SfA * SfAn001fG * SfC * SmA * SfG * SmA | 299 | GAUCAAGCAGA | SSnX SSnX SSSSS |
| 19989 | * SfG * SmA * SfA * SfA * SfG * SfCn001fC * SfA * SfG | | GAAAGCCAG | SSSSS nX SS |
| WV- | fG * SfA * SfUn001fC * SfA * SfAn001fG * SfC * SmA * SfG * SmA | 300 | GAUCAAGCAGA | SSnX SSnX SSSSS |
| 19989 | * SfG * SmA * SfA * SfA * SfG * SfCn001fC * SfA * SfG | | GAAAGCCAG | SSSSS nX SS |
| WV- | fA * SfU * SfCn001fA * SfA * SfGn001fC * SfA * SmG * SfA * SmG | 301 | AUCAAGCAGAG | SSnX SSnX SSSSS |
| 19990 | * SfA * SmA * SfA * SfG * SfC * SfCn001fA * SfG * SfU | | AAAGCCAGU | SSSSS nX SS |
| WV- | fU * SfC * SfAn001fA * SfG * SfCn001fA * SfG * SmA * SfG * SmA | 302 | UCAAGCAGAGA | SSnX SSnX SSSSS |
| 19991 | * SfA * SmA * SfG * SfC * SfC * SfAn001fG * SfU * SfC | | AAGCCAGUC | SSSSS nX SS |
| WV- | fC * SfA * SfAn001fG * SfC * SfAn001fG * SfA * SmG * SfA * SmA | 303 | CAAGCAGAGAA | SSnX SSnX SSSSS |
| 19992 | * SfA * SmG * SfC * SfC * SfA * SfGn001fU * SfC * SfG | | AGCCAGUCG | SSSSS nX SS |
| WV- | fA * SfA * SfGn001fC * SfA * SfGn001fA * SfG * SmA * SfA * SmA | 304 | AAGCAGAGAAA | SSnX SSnX SSSSS |
| 19993 | * SfG * SmC * SfC * SfA * SfG * SfUn001fC * SfG * SfG | | GCCAGUCGG | SSSSS nX SS |
| WV- | fA * SfG * SfCn001fA * SfG * SfAn001fG * SfA * SmA * SfA * SmG | 305 | AGCAGAGAAAG | SSnX SSnX SSSSS |
| 19994 | * SfC * SmC * SfA * SfG * SfU * SfCn001fG * SfG * SfU | | CCAGUCGGU | SSSSS nX SS |
| WV- | fG * SfC * SfAn001fG * SfA * SfGn001fA * SfA * SmA * SfG * SmC | 306 | GCAGAGAAAGC | SSnX SSnX SSSSS |
| 19995 | * SfC * SmA * SfG * SfU * SfC * SfGn001fG * SfU * SfA | | CAGUCGGUA | SSSSS nX SS |
| WV- | fC * SfA * SfGn001fA * SfG * SfAn001fA * SfA * SmG * SfC * SmC | 307 | CAGAGAAAGCC | SSnX SSnX SSSSS |
| 19996 | * SfA * SmG * SfU * SfC * SfG * SfGn001fU * SfA * SfA | | AGUCGGUAA | SSSSS nX SS |
| WV- | fA * SfG * SfAn001fG * SfA * SfAn001fA * SfG * SmC * SfC * SmA | 308 | AGAGAAAGCCA | SSnX SSnX SSSSS |
| 19997 | * SfG * SmU * SfC * SfG * SfG * SfUn001fA * SfA * SfG | | GUCGGUAAG | SSSSS nX SS |
| WV- | fG * SfA * SfGn001fA * SfA * SfAn001fG * SfC * SmC * SfA * SmG | 309 | GAGAAAGCCAG | SSnX SSnX SSSSS |
| 19998 | * SfU * SmC * SfG * SfG * SfU * SfAn001fA * SfG * SfU | | UCGGUAAGU | SSSSS nX SS |
| WV- | fA * SfG * SfAn001fA * SfA * SfGn001fC * SfC * SmA * SfG * SmU | 310 | AGAAAGCCAGU | SSnX SSnX SSSSS |
| 19999 | * SfC * SmG * SfG * SfU * SfA * SfAn001fG * SfU * SfU | | CGGUAAGUU | SSSSS nX SS |
| WV- | fG * SfA * SfAn001fA * SfG * SfCn001fC * SfA * SmG * SfU * SmC | 311 | GAAAGCCAGUC | SSnX SSnX SSSSS |
| 20000 | * SfG * SmG * SfU * SfA * SfA * SfGn001fU * SfU * SfC | | GGUAAGUUC | SSSSS nX SS |
| WV- | fA * SfA * SfAn001fG * SfC * SfCn001fA * SfG * SmU * SfC * SmG | 312 | AAAGCCAGUCG | SSnX SSnX SSSSS |
| 20001 | * SfG * SmU * SfA * SfA * SfG * SfUn001fU * SfC * SfU | | GUAAGUUCU | SSSSS nX SS |
| WV- | fA * SfA * SfGn001fC * SfC * SfAn001fG * SfU * SmC * SfG * SmG | 313 | AAGCCAGUCGG | SSnX SSnX SSSSS |
| 20002 | * SfU * SmA * SfA * SfG * SfU * SfUn001fC * SfU * SfG | | UAAGUUCUG | SSSSS nX SS |
| WV- | fA * SfG * SfCn001fC * SfA * SfGn001fU * SfC * SmG * SfG * SmU | 314 | AGCCAGUCGGU | SSnX SSnX SSSSS |
| 20003 | * SfA * SmA * SfG * SfU * SfU * SfCn001fU * SfG * SfU | | AAGUUCUGU | SSSSS nX SS |
| WV- | fG * SfC * SfCn001fA * SfG * SfUn001fC * SfG * SmG * SfU * SmA | 315 | GCCAGUCGGUA | SSnX SSnX SSSSS |
| 20004 | * SfA * SmG * SfU * SfU * SfC * SfU n001fG * SfU * SfC | | AGUUCUGUC | SSSSS nX SS |
| WV- | fC * SfC * SfAn001fG * SfU * SfCn001fG * SfG * SmU * SfA * SmA | 316 | CCAGUCGGUAA | SSnX SSnX SSSSS |
| 20005 | * SfG * SmU * SfU * SfC * SfU * SfGn001fU * SfC * SfC | | GUUCUGUCC | SSSSS nX SS |
| WV- | fC * SfA * SfGn001fU * SfC * SfGn001fG * SfU * SmA * SfA * SmG | 317 | CAGUCGGUAAG | SSnX SSnX SSSSS |
| 20006 | * SfU * SmU * SfC * SfU * SfG * SfUn001fC * SfC * SfA | | UUCUGUCCA | SSSSS nX SS |
| WV- | fA * SfG * SfUn001fC * SfG * SfGn001fU * SfA * SmA * SfG * SmU | 318 | AGUCGGUAAGU | SSnX SSnX SSSSS |
| 20007 | * SfU * SmC * SfU * SfG * SfU * SfCn001fC * SfA * SfA | | UCUGUCCAA | SSSSS nX SS |
| WV- | fG * SfU * SfCn001fG * SfG * SfUn001fA * SfA * SmG * SfU * SmU | 319 | GUCGGUAAGUU | SSnX SSnX SSSSS |
| 20008 | * SfC * SmU * SfG * SfU * SfC * SfCn001fA * SfA * SfG | | CUGUCCAAG | SSSSS nX SS |
| WV- | fU * SfC * SfGn001fG * SfU * SfAn001fA * SfG * SmU * SfU * SmC | 320 | UCGGUAAGUUC | SSnX SSnX SSSSS |
| 20009 | * SfU * SmG * SfU * SfC * SfC * SfAn001fA * SfG * SfC | | UGUCCAAGC | SSSSS nX SS |
| WV- | fC * SfG * SfGn001fU * SfA * SfAn001fG * SfU * SmU * SfC * SmU | 321 | CGGUAAGUUCU | SSnX SSnX SSSSS |
| 20010 | * SfG * SmU * SfC * SfC * SfA * SfAn001fG * SfC * SfC | | GUCCAAGCC | SSSSS nX SS |
| WV- | fC * SfG * SfGn001fU * SfA * SfAn001fG * SfU * SmU * SfC * SmU | 322 | CGGUAAGUUCU | SSnX SSnX SSSSS |
| 20010 | * SfG * SmU * SfC * SfC * SfA * SfAn001fG * SfC * SfC | | GUCCAAGCC | SSSSS nX SS |
| WV- | fG * SfU * SfAn001fA * SfG * SfUn001fU * SfC * SmU * SfG * SmU | 323 | GUAAGUUCUGU | SSnX SSnX SSSSS |
| 20012 | * SfC * SmC * SfA * SfA * SfG * SfCn001fC * SfC * SfG | | CCAAGCCCG | SSSSS nX SS |
| WV- | fU * SfA * SfAn001fG * SfU * SfUn001fC * SfU * SmG * SfU * SmC | 324 | UAAGUUCUGUC | SSnX SSnX SSSSS |
| 20013 | * SfC * SmA * SfA * SfG * SfC * SfCn001fC * SfG * SfG | | CAAGCCCGG | SSSSS nX SS |
| WV- | fA * SfA * SfGn001fU * SfU * SfCn001fU * SfG * SmU * SfC * SmC | 325 | AAGUUCUGUCC | SSnX SSnX SSSSS |
| 20014 | * SfA * SmA * SfG * SfC * SfC * SfCn001fG * SfG * SfU | | AAGCCCGGU | SSSSS nX SS |
| WV- | fA * SfG * SfUn001fU * SfC * SfUn001fG * SfU * SmC * SfC * SmA | 326 | AGUUCUGUCCA | SSnX SSnX SSSSS |
| 20015 | * SfA * SmG * SfC * SfC * SfC * SfGn001fG * SfU * SfU | | AGCCCGGUU | SSSSS nX SS |
| WV- | fA * SfG * SfUn001fU * SfC * SfUn001fG * SfU * SmC * SfC * SmA | 327 | AGUUCUGUCCA | SSnX SSnX SSSSS |
| 20015 | * SfA * SmG * SfC * SfC * SfC * SfGn001fG * SfU * SfU | | AGCCCGGUU | SSSSS nX SS |
| WV- | fG * SfU * SfUn001fC * SfU * SfGn001fU * SfC * SmC * SfA * SmA | 328 | GUUCUGUCCAA | SSnX SSnX SSSSS |
| 20016 | * SfG * SmC * SfC * SfC * SfG * SfGn001fU * SfU * SfG | | GCCCGGUUG | SSSSS nX SS |
| WV- | fU * SfU * SfCn001fU * SfG * SfUn001fC * SfC * SmA * SfA * SmG | 329 | UUCUGUCCAAG | SSnX SSnX SSSSS |
| 20017 | * SfC * SmC * SfC * SfG * SfG * SfUn001fU * SfG * SfA | | CCCGGUUGA | SSSSS nX SS |
| WV- | fU * SfC * SfUn001fG * SfU * SfCn001fC * SfA * SmA * SfG * SmC | 330 | UCUGUCCAAGC | SSnX SSnX SSSSS |
| 20018 | * SfC * SmC * SfG * SfG * SfU * SfUn001fG * SfA * SfA | | CCGGUUGAA | SSSSS nX SS |
| WV- | fC * SfU * SfGn001fU * SfC * SfCn001fA * SfA * SmG * SfC * SmC | 331 | CUGUCCAAGCC | SSnX SSnX SSSSS |
| 20019 | * SfC * SmG * SfG * SfU * SfU * SfGn001fA * SfA * SfA | | CGGUUGAAA | SSSSS nX SS |
| WV- | fU * SfG * SfUn001fC * SfC * SfAn001fA * SfG * SmC * SfC * SmC | 332 | UGUCCAAGCCC | SSnX SSnX SSSSS |
| 20020 | * SfG * SmG * SfU * SfU * SfG * SfAn001fA * SfA * SfU | | GGUUGAAAU | SSSSS nX SS |
| WV- | fG * SfU * SfCn001fC * SfA * SfAn001fG * SfC * SmC * SfC * SmG | 333 | GUCCAAGCCCG | SSnX SSnX SSSSS |
| 20021 | * SfG * SmU * SfU * SfG * SfA * SfAn001fA * SfU * SfC | | GUUGAAAUC | SSSSS nX SS |
| WV- | fU * SfC * SfCn001fA * SfA * SfGn001fC * SfC * SmC * SfG * SmG | 334 | UCCAAGCCCGG | SSnX SSnX SSSSS |
| 20022 | * SfU * SmU * SfG * SfA * SfA * SfAn001fU * SfC * SfU | | UUGAAAUCU | SSSSS nX SS |
| WV- | fC * SfC * SfAn001fA * SfG * SfCn001fC * SfC * SmG * SfG * SmU | 335 | CCAAGCCCGGU | SSnX SSnX SSSSS |
| 20023 | * SfU * SmG * SfA * SfA * SfA * SfUn001fC * SfU * SfG | | UGAAAUCUG | SSSSS nX SS |
| WV- | fC * SfA * SfAn001fG * SfC * SfCn001fC * SfG * SmG * SfU * SmU | 336 | CAAGCCCGGUU | SSnX SSnX SSSSS |
| 20024 | * SfG * SmA * SfA * SfA * SfU * SfCn001fU * SfG * SfC | | GAAAUCUGC | SSSSS nX SS |
| WV- | fA * SfA * SfGn001fC * SfC * SfCn001fG * SfG * SmU * SfU * SmG | 337 | AAGCCCGGUUG | SSnX SSnX SSSSS |
| 20025 | * SfA * SmA * SfA * SfU * SfC * SfUn001fG * SfC * SfC | | AAAUCUGCC | SSSSS nX SS |
| WV- | fA * SfG * SfCn001fC * SfC * SfGn001fG * SfU * SmU * SfG * SmA | 338 | AGCCCGGUUGA | SSnX SSnX SSSSS |
| 20026 | * SfA * SmA * SfU * SfC * SfU * SfGn001fC * SfC * SfA | | AAUCUGCCA | SSSSS nX SS |
| WV- | fG * SfC * SfCn001fC * SfG * SfGn001fU * SfU * SmG * SfA * SmA | 339 | GCCCGGUUGAA | SSnX SSnX SSSSS |
| 20027 | * SfA * SmU * SfC * SfU * SfG * SfCn001fC * SfA * SfG | | AUCUGCCAG | SSSSS nX SS |
| WV- | fC * SfC * SfCn001fG * SfG * SfUn001fU * SfG * SmA * SfA * SmA | 340 | CCCGGUUGAAA | SSnX SSnX SSSSS |
| 20028 | * SfU * SmC * SfU * SfG * SfC * SfCn001fA * SfG * SfA | | UCUGCCAGA | SSSSS nX SS |
| WV- | fC * SfC * SfGn001fG * SfU * SfUn001fG * SfA * SmA * SfA * SmU | 341 | CCGGUUGAAAU | SSnX SSnX SSSSS |
| 20029 | * SfC * SmU * SfG * SfC * SfC * SfAn001fG * SfA * SfG | | CUGCCAGAG | SSSSS nX SS |
| WV- | fC * SfG * SfGn001fU * SfU * SfGn001fA * SfA * SmA * SfU * SmC | 342 | CGGUUGAAAUC | SSnX SSnX SSSSS |
| 20030 | * SfU * SmG * SfC * SfC * SfA * SfGn001fA * SfG * SfC | | UGCCAGAGC | SSSSS nX SS |
| WV- | fG * SfG * SfUn001fU * SfG * SfAn001fA * SfA * SmU * SfC * SmU | 343 | GGUUGAAAUCU | SSnX SSnX SSSSS |
| 20031 | * SfG * SmC * SfC * SfA * SfG * SfAn001fG * SfC * SfA | | GCCAGAGCA | SSSSS nX SS |
| WV- | fG * SfU * SfUn001fG * SfA * SfAn001fA * SfU * SmC * SfU * SmG | 344 | GUUGAAAUCUG | SSnX SSnX SSSSS |
| 20032 | * SfC * SmC * SfA * SfG * SfA * SfGn001fC * SfA * SfG | | CCAGAGCAG | SSSSS nX SS |
| WV- | fU * SfU * SfGn001fA * SfA * SfAn001fU * SfC * SmU * SfG * SmC | 345 | UUGAAAUCUGC | SSnX SSnX SSSSS |
| 20033 | * SfC * SmA * SfG * SfA * SfG * SfCn001fA * SfG * SfG | | CAGAGCAGG | SSSSS nX SS |
| WV- | fG * SfA * SfAn001fA * SfU * SfCn001fU * SfG * SmC * SfC * SmA | 346 | GAAAUCUGCCA | SSnX SSnX SSSSS |
| 20035 | * SfG * SmA * SfG * SfC * SfA * SfGn001fG * SfU * SfA | | GAGCAGGUA | SSSSS nX SS |
| WV- | fA * SfA * SfAn001fU * SfC * SfUn001fG * SfC * SmC * SfA * SmG | 347 | AAAUCUGCCAG | SSnX SSnX SSSSS |
| 20036 | * SfA * SmG * SfC * SfA * SfG * SfGn001fU * SfA * SfC | | AGCAGGUAC | SSSSS nX SS |
| WV- | fA * SfU * SfCn001fU * SfG * SfCn001fC * SfA * SmG * SfA * SmG | 348 | AUCUGCCAGAG | SSnX SSnX SSSSS |
| 20038 | * SfC * SmA * SfG * SfG * SfU * SfAn001fC * SfC * SfU | | CAGGUACCU | SSSSS nX SS |
| WV- | fU * SfC * SfUn001fG * SfC * SfCn001fA * SfG * SmA * SfG * SmC | 349 | UCUGCCAGAGC | SSnX SSnX SSSSS |
| 20039 | * SfA * SmG * SfG * SfU * SfA * SfCn001fC * SfU * SfC | | AGGUACCUC | SSSSS nX SS |
| WV- | fU * SfG * SfCn001fC * SfA * SfGn001fA * SfG * SmC * SfA * SmG | 350 | UGCCAGAGCAG | SSnX SSnX SSSSS |
| 20041 | * SfG * SmU * SfA * SfC * SfC * SfUn001fC * SfC * SfA | | GUACCUCCA | SSSSS nX SS |
| WV- | fG * SfC * SfCn001fA * SfG * SfAn001fG * SfC * SmA * SfG * SmG | 351 | GCCAGAGCAGG | SSnX SSnX SSSSS |
| 20042 | * SfU * SmA * SfC * SfC * SfU * SfCn001fC * SfA * SfA | | UACCUCCAA | SSSSS nX SS |
| WV- | fC * SfA * SfGn001fA * SfG * SfCn001fA * SfG * SmG * SfU * SmA | 352 | CAGAGCAGGUA | SSnX SSnX SSSSS |
| 20044 | * SfC * SmC * SfU * SfC * SfC * SfAn001fA * SfC * SfA | | CCUCCAACA | SSSSS nX SS |
| WV- | fA * SfG * SfAn001fG * SfC * SfAn001fG * SfG * SmU * SfA * SmC | 353 | AGAGCAGGUAC | SSn SSnX SSSSS |
| 20045 | * SfC * SmU * SfC * SfC * SfA * SfAn001fC * SfA * SfU | | CUCCAACAU | SSSSS nX SS |
| WV- | fA * SfG * SfCn001fA * SfG * SfGn001fU * SfA * SmC * SfC * SmU | 354 | AGCAGGUACCU | SSnX SSnX SSSSS |
| 20047 | * SfC * SmC * SfA * SfA * SfC * SfAn001fU * SfC * SfA | | CCAACAUCA | SSSSS nX SS |
| WV- | fG * SfC * SfAn001fG * SfG * SfUn001fA * SfC * SmC * SfU * SmC | 355 | GCAGGUACCUC | SSnX SSnX SSSSS |
| 20048 | * SfC * SmA * SfA * SfC * SfA * SfUn001fC * SfA * SfA | | CAACAUCAA | SSSSS nX SS |
| WV- | fU * SfC * SfA * SfA * SfG * SfG * SfA * SfA * SmG * SfA * SmU * | 356 | UCAAGGAAGAU | SSSSS SSSSS SSSSS |
| 31179 | SfG * SmG * SfC * SfA * SfU * SfU * SfU * SfC * SfU | | GGCAUUUCU | SSSS |
| WV- | fC * SfA * SfA * SfG * SfG * SfU * SfC * SfA * SmC * SfC * SmC * | 357 | CAAGGUCACCC | SSSSS SSSSS SSSSS |
| 31180 | SfA * SmC * SfC * SfA * SfU * SfC * SfA * SfC * SfC | | ACCAUCACC | SSSS |
| WV- | fG * SfU * SfC * SfA * SfC * SfC * SfC * SfA * SmC * SfC * SmA * | 358 | GUCACCCACCA | SSSSS SSSSS SSSSS |
| 31181 | SfU * SmC * SfA * SfC * SfC * SfC * SfU * SfC * SfU | | UCACCCUCU | SSSS |
| WV- | fU * SfC * SfA * SfC * SfC * SfC * SfA * SfC * SmC * SfA * SmU * | 359 | UCACCCACCAU | SSSSS SSSSS SSSSS |
| 31182 | SfC * SmA * SfC * SfC * SfC * SfU * SfC * SfU * SfG | | CACCCUCUG | SSSS |
| WV- | fC * SfA * SfC * SfC * SfC * SfA * SfC * SfC * SmA * SfU * SmC * | 360 | CACCCACCAUC | SSSSS SSSSS SSSSS |
| 31183 | SfA * SmC * SfC * SfC * SfU * SfC * SfU * SfG * SfU | | ACCCUCUGU | SSSS |
| WV- | fG * SfA * SfU * SfC * SfA * SfA * SfG * SfC * SmA * SfG * SmA * | 361 | GAUCAAGCAGA | SSSSS SSSSS SSSSS |
| 31184 | SfG * SmA * SfA * SfA * SfG * SfC * SfC * SfA * SfG | | GAAAGCCAG | SSSS |
| WV- | fC * SfA * SfA * SfG * SfC * SfA * SfG * SfA * SmG * SfA * SmA * | 362 | CAAGCAGAGAA | SSSSS SSSSS SSSSS |
| 31185 | SfA * SmG * SfC * SfC * SfA * SfG * SfU * SfC * SfG | | AGCCAGUCG | SSSS |
| WV- | fA * SfA * SfG * SfC * SfC * SfA * SfG * SfU * SmC * SfG * SmG * | 363 | AAGCCAGUCGG | SSSSS SSSSS SSSSS |
| 31186 | SfU * SmA * SfA * SfG * SfU * SfU * SfC * SfU * SfG | | UAAGUUCUG | SSSS |
| WV- | fA * SfG * SfU * SfC * SfG * SfG * SfU * SfA * SmA * SfG * SmU * | 364 | AGUCGGUAAGU | SSSSS SSSSS SSSSS |
| 31187 | SfU * SmC * SfU * SfG * SfU * SfC * SfC * SfA * SfA | | UCUGUCCAA | SSSS |
| WV- | fG * SfU * SfC * SfG * SfG * SfU * SfA * SfA * SmG * SfU * SmU * | 365 | GUCGGUAAGUU | SSSSS SSSSS SSSSS |
| 31188 | SfC * SmU * SfG * SfU * SfC * SfC * SfA * SfA * SfG | | CUGUCCAAG | SSSS |
| WV- | fU * SfC * SfG * SfG * SfU * SfA * SfA * SfG * SmU * SfU * SmC * | 366 | UCGGUAAGUUC | SSSSS SSSSS SSSSS |
| 31189 | SfU * SmG * SfU * SfC * SfC * SfA * SfA * SfG * SfC | | UGUCCAAGC | SSSS |
| WV- | fC * SfG * SfG * SfU * SfA * SfA * SfG * SfU * SmU * SfC * SmU * | 367 | CGGUAAGUUCU | SSSSS SSSSS SSSSS |
| 31190 | SfG * SmU * SfC * SfC * SfA * SfA * SfG * SfC * SfC | | GUCCAAGCC | SSSS |
| WV- | fG * SfG * SfU * SfA * SfA * SfG * SfU * SfU * SmC * SfU * SmG * | 368 | GGUAAGUUCUG | SSSSS SSSSS SSSSS |
| 31191 | SfU * SmC * SfC * SfA * SfA * SfG * SfC * SfC * SfC | | UCCAAGCCC | SSSS |
| WV- | fG * SfU * SfA * SfA * SfG * SfU * SfU * SfC * SmU * SfG * SmU * | 369 | GUAAGUUCUGU | SSSSS SSSSS SSSSS |
| 31192 | SfC * SmC * SfA * SfA * SfG * SfC * SfC * SfC * SfG | | CCAAGCCCG | SSSS |
| WV- | fU * SfA * SfA * SfG * SfU * SfU * SfC * SfU * SmG * SfU * SmC * | 370 | UAAGUUCUGUC | SSSSS SSSSS SSSSS |
| 31193 | SfC * SmA * SfA * SfG * SfC * SfC * SfC * SfG * SfG | | CAAGCCCGG | SSSS |
| WV- | fA * SfA * SfG * SfU * SfU * SfC * SfU * SfG * SmU * SfC * SmC * | 371 | AAGUUCUGUCC | SSSSS SSSSS SSSSS |
| 31194 | SfA * SmA * SfG * SfC * SfC * SfC * SfG * SfG * SfU | | AAGCCCGGU | SSSS |
| WV- | fA * SfG * SfU * SfU * SfC * SfU * SfG * SfU * SmC * SfC * SmA * | 372 | AGUUCUGUCCA | SSSSS SSSSS SSSSS |
| 31195 | SfA * SmG * SfC * SfC * SfC * SfG * SfG * SfU * SfU | | AGCCCGGUU | SSSS |
| WV- | fG * SfU * SfU * SfC * SfU * SfG * SfU * SfC * SmC * SfA * SmA * | 373 | GUUCUGUCCAA | SSSSS SSSSS SSSSS |
| 31196 | SfG * SmC * SfC * SfC * SfG * SfG * SfU * SfU * SfG | | GCCCGGUUG | SSSS |
| WV- | fU * SfU * SfC * SfU * SfG * SfU * SfC * SfC * SmA * SfA * SmG * | 374 | UUCUGUCCAAG | SSSSS SSSSS SSSSS |
| 31197 | SfC * SmC * SfC * SfG * SfG * SfU * SfU * SfG * SfA | | CCCGGUUGA | SSSS |
| WV- | fU * SfC * SfU * SfG * SfU * SfC * SfC * SfA * SmA * SfG * SmC * | 375 | UCUGUCCAAGC | SSSSS SSSSS SSSSS |
| 31198 | SfC * SmC * SfG * SfG * SfU * SfU * SfG * SfA * SfA | | CCGGUUGAA | SSSS |
| WV- | fG * SfU * SfC * SfC * SfA * SfA * SfG * SfC * SmC * SfC * SmG * | 376 | GUCCAAGCCCG | SSSSS SSSSS SSSSS |
| 31199 | SfG * SmU * SfU * SfG * SfA * SfA * SfA * SfU * SfC | | GUUGAAAUC | SSSS |
| WV- | fG * SfC * SfA * SfU * SfU * SfU * SfC * SfU * SmA * SfG * SmU * | 377 | GCAUUUCUAGU | SSSSS SSSSS SSSSS |
| 31213 | SfU * SmU * SfG * SfG * SfA * SfG * SfA * SfU * SfG | | UUGGAGAUG | SSSS |
| WV- | fG * SfG * SfC * SfA * SfG * SfU * SfU * SfU * SmC * SfC * SmU * | 378 | GGCAGUUUCCU | SSSSS SSSSS SSSSS |
| 31215 | SfU * SmA * SfG * SfU * SfA * SfA * SfC * SfC * SfA | | UAGUAACCA | SSSS |
| WV- | fG * SfC * SfA * SfG * SfU * SfU * SfU * SfC * SmC * SfU * SmU * | 379 | GCAGUUUCCUU | SSSSS SSSSS SSSSS |
| 31216 | SfA * SmG * SfU * SfA * SfA * SfC * SfC * SfA * SfC | | AGUAACCAC | SSSS |
| WV- | fC * SfA * SfG * SfU * SfU * SfU * SfC * SfC * SmU * SfU * SmA * | 380 | CAGUUUCCUUA | SSSSS SSSSS SSSSS |
| 31217 | SfG * SmU * SfA * SfA * SfC * SfC * SfA * SfC * SfA | | GUAACCACA | SSSS |
| WV- | fA * SfG * SfU * SfU * SfU * SfC * SfC * SfU * SmU * SfA * SmG * | 381 | AGUUUCCUUAG | SSSSS SSSSS SSSSS |
| 31218 | SfU * SmA * SfA * SfC * SfC * SfA * SfC * SfA * SfG | | UAACCACAG | SSSS |
| WV- | fG * SfU * SfU * SfU * SfC * SfC * SfU * SfU * SmA * SfG * SmU * | 382 | GUUUCCUUAGU | SSSSS SSSSS SSSSS |
| 31219 | SfA * SmA * SfC * SfC * SfA * SfC * SfA * SfG * SfG | | AACCACAGG | SSSS |
| WV- | fU * SfU * SfU * SfC * SfC * SfU * SfU * SfA * SmG * SfU * SmA * | 383 | UUUCCUUAGUA | SSSSS SSSSS SSSSS |
| 31220 | SfA * SmC * SfC * SfA * SfC * SfA * SfG * SfG * SfU | | ACCACAGGU | SSSS |
| WV- | fU * SfU * SfC * SfC * SfU * SfU * SfA * SfG * SmU * SfA * SmA * | 384 | UUCCUUAGUAA | SSSSS SSSSS SSSSS |
| 31221 | SfC * SmC * SfA * SfC * SfA * SfG * SfG * SfU * SfU | | CCACAGGUU | SSSS |
| WV- | fU * SfC * SfC * SfU * SfU * SfA * SfG * SfU * SmA * SfA * SmC * | 385 | UCCUUAGUAAC | SSSSS SSSSS SSSSS |
| 31222 | SfC * SmA * SfC * SfA * SfG * SfG * SfU * SfU * SfG | | CACAGGUUG | SSSS |
| WV- | fC * SfC * SfU * SfU * SfA * SfG * SfU * SfA * SmA * SfC * SmC * | 386 | CCUUAGUAACC | SSSSS SSSSS SSSSS |
| 31223 | SfA * SmC * SfA * SfG * SfG * SfU * SfU * SfG * SfU | | ACAGGUUGU | SSSS |
| WV- | fG * SfU * SfU * SfG * SfU * SfG * SfU * SfC * SmA * SfC * SmC * | 387 | GUUGUGUCACC | SSSSS SSSSS SSSSS |
| 31224 | SfA * SmG * SfA * SfG * SfU * SfA * SfA * SfC * SfA | | AGAGUAACA | SSSS |
| WV- | fU * SfU * SfG * SfU * SfG * SfU * SfC * SfA * SmC * SfC * SmA * | 388 | UUGUGUCACCA | SSSSS SSSSS SSSSS |
| 31225 | SfG * SmA * SfG * SfU * SfA * SfA * SfC * SfA * SfG | | GAGUAACAG | SSSS |
| WV- | fU * SfG * SfU * SfG * SfU * SfC * SfA * SfC * SmC * SfA * SmG * | 389 | UGUGUCACCAG | SSSSS SSSSS SSSSS |
| 31226 | SfA * SmG * SfU * SfA * SfA * SfC * SfA * SfG * SfU | | AGUAACAGU | SSSS |
| WV- | fG * SfUn001RfU * SfU * SfCn001RfC * SfU * SmUfA * SmG * SfU | 390 | GUUUCCUUAGU | SnRSSnRSS OSSS |
| 32693 | * SmAfA * SfC * SfC * SfAn001RfC * SfA * SfG | | AACCACAG | OSSSnRSS |
| WV- | fG * SfUn001RfU * SfU * SfCn001RmCfU * SfU * SmA * SfG * | 391 | GUUUCCUUAGU | SnRSSnR OSSSS |
| 32694 | SmUmA * SfA * SfC * SfC * SfAn001RfC * SfA * SfG | | AACCACAG | OSSSSnRSS |

Spaces in Table A1 are utilized for formatting and readability, e.g., OXXXXX XXXXX XXXXX XXXX illustrates the same stereochemistry as OXXXXXXXXXXXXXXXXXXX; * S and *S both indicate phosphorothioate internucleotidic linkage wherein the linkage phosphorus has Sp configuration; etc.

All DMD oligonucleotides listed in Tables A1 are single-stranded. As described in the present application, they may be used as a single strand, or as a strand to form complexes with one or more other strands.

Some sequences, due to their length, are divided into multiple lines.

As appreciated by those skilled in the art, nucleoside units are unmodified and contain unmodified nucleobases and 2′-deoxy sugars (two 2′-H) unless otherwise indicated (e.g., with r, m, m5, eo, etc.); linkages, unless otherwise indicated, are natural phosphate linkages; and acidic/basic groups may independently exist in their salt forms.
ID: Identification number for an oligonucleotide.
WV-13405, WV-13406 and WV-13407 are fully PMO (morpholino oligonucleotides).
n001: non-negatively charged linkage

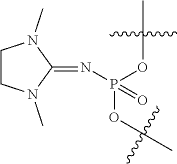 [SMILES: *OP(=O)(N=C1N(C)CCN1C)O*]

(which is stereorandom unless otherwise indicated (e.g., as n001R, or n001S));
n001R: n001 being chirally controlled and having the Rp configuration;
n001S: n001 being chirally controlled and having the Sp configuration;
nX: in Linkage/Stereochemistry, nO or nX indicates a stereorandom n001;
nR: in Linkage/Stereochemistry, nR indicates n001 being chirally controlled and having the Rp configuration;
nS: in Linkage/Stereochemistry, nS indicates n001 being chirally controlled and having the Sp configuration;
F, f: 2′-F modification on the following nucleoside (e.g., fA

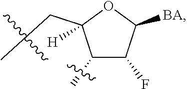

wherein BA is nucleobase A));
m: 2′-OMe modification on the following nucleoside (e.g., mA

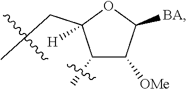

wherein BA is nucleobase A));
*, PS: Phosphorothioate;
*R, R, Rp: Phosphorothioate in Rp conformation;
*S, S, Sp: Phosphorothioate in Sp conformation;
X: Phosphorothioate stereorandom;
O, PO: phosphodiester (phosphate). When no internucleotidic linkage is specified between two nucleoside units, the internucleotidic linkage is a phosphodiester linkage (natural phosphate linkage).

In some embodiments, each phosphorothioate internucleotidic linkage of a DMD oligonucleotide is independently a chirally controlled internucleotidic linkage. In some embodiments, a provided DMD oligonucleotide composition is a chirally controlled DMD oligonucleotide composition of a DMD oligonucleotide type listed in Table A1, wherein each phosphorothioate internucleotidic linkage of the DMD oligonucleotide is independently a chirally controlled internucleotidic linkage.

In some embodiments, the present disclosure provides compositions comprising or consisting of a plurality of provided DMD oligonucleotides (e.g., chirally controlled DMD oligonucleotide compositions). In some embodiments, all DMD oligonucleotides of the plurality are of the same type, i.e., all have the same base sequence, pattern of backbone linkages, pattern of backbone chiral centers, and pattern of backbone phosphorus modifications. In some embodiments, all DMD oligonucleotides of the same type are structural identical. In some embodiments, provided compositions comprise DMD oligonucleotides of a plurality of DMD oligonucleotides types, typically in controlled amounts. In some embodiments, a provided chirally controlled DMD oligonucleotide composition comprises a combination of two or more provided DMD oligonucleotide types.

In some embodiments, a DMD oligonucleotide composition of the present disclosure is a chirally controlled DMD oligonucleotide composition, wherein the sequence of the DMD oligonucleotides of its plurality comprises or consists of a base sequence listed in Table A1.

In some embodiments, base sequences of oligonucleotides are or comprise a sequence described in Table A1. In some embodiments, a base sequence is or comprises AGUUUCCUUAGUAACCACAG (SEQ ID NO: 392). In some embodiments, a base sequence is or comprises UGGCAUUUCUAGUUUGGAGA (SEQ ID NO: 393). In some embodiments, a base sequence is or comprises GGUAAGUUCUGUCCAAGCCC (SEQ ID NO: 394). In some embodiments, a base sequence is or comprises GGUAAGUUCUGUCCAAGCCC (SEQ ID NO: 394). In some embodiments, a base sequence is or comprises AUGGCAUUUCUAGUUUGGAG (SEQ ID NO: 395). In some embodiments, a base sequence is or comprises GCAUUUCUAGUUUGGAGAUG (SEQ ID NO: 396). In some embodiments, a base sequence is or comprises CAGUUUCCUUAGUAACCACA (SEQ ID NO: 397). In some embodiments, a base sequence is or comprises UUCCUUAGUAACCACAGGUU (SEQ ID NO: 398). In some embodiments, a base sequence is or comprises GUACCUCCAACAUCAAGGAA (SEQ ID NO: 399). In some embodiments, a base sequence is or comprises GGCAUUUCUAGUUUGGAGAU (SEQ ID NO: 400). In some embodiments, a base sequence is or comprises UGGCAGUUUCCUUAGUAACC (SEQ ID NO: 401). In some embodiments, a base sequence is or comprises GGUAAGUUCUGUCCAAGCCC (SEQ ID NO: 394). In some embodiments, a base sequence is or comprises CAACAUCAAGGAAGAUGGCA (SEQ ID NO: 407). In some embodiments, a base sequence is or comprises AUGGCAUUUCUAGUUUGGAG (SEQ ID NO: 395).

In some embodiments, the present disclosure provides a DMD oligonucleotide composition, wherein the DMD oligonucleotide is WV-20011.

In some embodiments, the present disclosure provides a DMD oligonucleotide composition, wherein the DMD oligonucleotide is WV-20052.

In some embodiments, the present disclosure provides a DMD oligonucleotide composition, wherein the DMD oligonucleotide is WV-20059.

In some embodiments, the present disclosure provides a DMD oligonucleotide composition, wherein the DMD oligonucleotide is WV-20072.

In some embodiments, the present disclosure provides a DMD oligonucleotide composition, wherein the DMD oligonucleotide is WV-20073.

In some embodiments, the present disclosure provides a DMD oligonucleotide composition, wherein the DMD oligonucleotide is WV-20074.

In some embodiments, the present disclosure provides a DMD oligonucleotide composition, wherein the DMD oligonucleotide is WV-20075.

In some embodiments, the present disclosure provides a DMD oligonucleotide composition, wherein the DMD oligonucleotide is WV-20076.

In some embodiments, the present disclosure provides a DMD oligonucleotide composition, wherein the DMD oligonucleotide is WV-20096.

In some embodiments, the present disclosure provides a DMD oligonucleotide composition, wherein the DMD oligonucleotide is WV-20097.

In some embodiments, the present disclosure provides a DMD oligonucleotide composition, wherein the DMD oligonucleotide is WV-20101.

In some embodiments, the present disclosure provides a DMD oligonucleotide composition, wherein the DMD oligonucleotide is WV-20119.

In some embodiments, the present disclosure provides a chirally controlled composition of DMD oligonucleotide WV-20011.

In some embodiments, the present disclosure provides a chirally controlled composition of DMD oligonucleotide WV-20052.

In some embodiments, the present disclosure provides a chirally controlled composition of DMD oligonucleotide WV-20059.

In some embodiments, the present disclosure provides a chirally controlled composition of DMD oligonucleotide WV-20072.

In some embodiments, the present disclosure provides a chirally controlled composition of DMD oligonucleotide WV-20073.

In some embodiments, the present disclosure provides a chirally controlled composition of DMD oligonucleotide WV-20074.

In some embodiments, the present disclosure provides a chirally controlled composition of DMD oligonucleotide WV-20075.

In some embodiments, the present disclosure provides a chirally controlled composition of DMD oligonucleotide WV-20076.

In some embodiments, the present disclosure provides a chirally controlled composition of DMD oligonucleotide WV-20096.

In some embodiments, the present disclosure provides a chirally controlled composition of DMD oligonucleotide WV-20097.

In some embodiments, the present disclosure provides a chirally controlled composition of DMD oligonucleotide WV-20101.

In some embodiments, the present disclosure provides a chirally controlled composition of DMD oligonucleotide WV-20119.

In some embodiments, the present disclosure provides a chirally controlled composition of DMD oligonucleotide WV-20011.

In some embodiments, the present disclosure provides a chirally controlled composition of DMD oligonucleotide WV-20052.

In some embodiments, the present disclosure provides a chirally controlled composition of DMD oligonucleotide WV-20059.

In some embodiments, the present disclosure provides a chirally controlled composition of DMD oligonucleotide WV-20072.

In some embodiments, the present disclosure provides a chirally controlled composition of DMD oligonucleotide WV-20073.

In some embodiments, the present disclosure provides a chirally controlled composition of DMD oligonucleotide WV-20074.

In some embodiments, the present disclosure provides a chirally controlled composition of DMD oligonucleotide WV-20075.

In some embodiments, the present disclosure provides a chirally controlled composition of DMD oligonucleotide WV-20076.

In some embodiments, the present disclosure provides a chirally controlled composition of DMD oligonucleotide WV-20096.

In some embodiments, the present disclosure provides a chirally controlled composition of DMD oligonucleotide WV-20097.

In some embodiments, the present disclosure provides a chirally controlled composition of DMD oligonucleotide WV-20101.

In some embodiments, the present disclosure provides a chirally controlled composition of DMD oligonucleotide WV-20119.

As described herein, in some embodiments, the present disclosure provides oligonucleotides and compositions (e.g., chirally controlled oligonucleotide compositions, pharmaceutically acceptable compositions, etc.) useful for preventing and/or treating a condition, disorder or disease (e.g., BMD, DMD, etc.) amenable to exon skipping, e.g., exon 51 skipping. In some embodiments, the present disclosure provides methods for preventing and/or treating a condition, disorder or disease (e.g., BMD, DMD, etc.) amenable to exon skipping, e.g., exon 51 skipping, comprising administering to a subject susceptible thereto or suffering therefrom a therapeutically effective amount of an oligonucleotide or a pharmaceutically acceptable salt thereof, or a composition. In some embodiments, an oligonucleotide may be administered in a composition comprising various forms of the oligonucleotide, e.g., a liquid composition comprising one or more dissolved acid and/or one or more salt forms of the oligonucleotide in a buffer system. In some embodiments, a salt is a sodium salt. In some embodiments, an oligonucleotide is WV-31582 or a pharmaceutically acceptable salt thereof. In some embodiments, an oligonucleotide is WV-31565 or a pharmaceutically acceptable salt thereof. In some embodiments, an oligonucleotide is WV-31568 or a pharmaceutically acceptable salt thereof. In some embodiments, an oligonucleotide is WV-31561 or a pharmaceutically acceptable salt thereof. In some embodiments, an oligonucleotide is WV-31576 or a pharmaceutically acceptable salt thereof. In some embodiments, an oligonucleotide is WV-31567 or a pharmaceutically acceptable salt thereof. In some embodiments, an oligonucleotide is WV-31569 or a pharmaceutically acceptable salt thereof. In some embodiments, an oligonucleotide is WV-31583 or a pharmaceutically acceptable salt thereof. In some embodiments, an oligonucleotide is WV-31562 or a pharmaceutically acceptable salt thereof. In some embodiments, an oligonucleotide is WV-31578 or a pharmaceutically acceptable salt thereof. In some embodiments, an oligonucleotide is WV-31580 or a pharmaceutically acceptable salt thereof. In some embodiments, an oligonucleotide is WV-31573 or a pharmaceutically acceptable salt thereof. In some embodiments, an oligonucleotide is WV-31563 or a pharmaceutically acceptable salt thereof. In some embodiments, an oligonucleotide is WV-31564 or a pharmaceutically acceptable salt thereof. In some embodiments, a salt is a sodium salt. In some embodiments, provided oligonucleotides are of high diastereopurity, e.g., 1%, 5%, 10%, 15%, 20%, 25%, 30%, 35%, 40%, 45%, 50%, 55%, 60%, 65%, 70%, 75%, 80%, 85%, 90%, 95%, or more. In some embodiments, it is at least 10%. In some embodiments, it is at least 20%. In some embodiments, it is at least 30%. In some embodiments, it is at least 40%. In some embodiments, it is at least 50%. In some embodiments, it is at least 60%. In some embodiments, it is at least 70%. In some embodiments, it is at least 80%. In some embodiments, it is at least 90%.

As described herein, in some embodiments, the present disclosure provides a chirally controlled oligonucleotide composition, wherein a level (e.g., at least about 1%, 5%, 10%, 15%, 20%, 25%, 30%, 35%, 40%, 45%, 50%, 55%, 60%, 65%, 70%, 75%, 80%, 85%, 90%, 95%, or more) of all oligonucleotides in the composition each independently have the structure of a single oligonucleotide or a salt thereof. In some embodiments, the present disclosure provides a chirally controlled oligonucleotide composition, wherein a level (e.g., at least about 1%, 5%, 10%, 15%, 20%, 25%, 30%, 35%, 40%, 45%, 50%, 55%, 60%, 65%, 70%, 75%, 80%, 85%, 90%, 95%, or more) of all oligonucleotides that share a common base sequence in the composition each independently have the structure of a single oligonucleotide or a salt thereof. In some embodiments, a level is at least 10%. In some embodiments, a level is at least 20%. In some embodiments, a level is at least 30%. In some embodiments, a level is at least 40%. In some embodiments, a level is at least 50%. In some embodiments, a level is at least 60%. In some embodiments, a level is at least 70%. In some embodiments, a level is at least 80%. In some embodiments, a level is at least 90%. In some embodiments, each salt is independently a pharmaceutically acceptable salt. In some embodiments, a salt is a sodium salt. In some embodiments, a single oligonucleotide is WV-31582. In some embodiments, a single oligonucleotide is WV-31565. In some embodiments, a single oligonucleotide is WV-31568. In some embodiments, a single oligonucleotide is WV-31561. In some embodiments, a single oligonucleotide is WV-31576. In some embodiments, a single oligonucleotide is WV-31567. In some embodiments, a single oligonucleotide is WV-31569. In some embodiments, a single oligonucleotide is WV-31583. In some embodiments, a single oligonucleotide is WV-31562. In some embodiments, a single oligonucleotide is WV-31578. In some embodiments, a single oligonucleotide is WV-31580. In some embodiments, a single oligonucleotide is WV-31573. In some embodiments, a single oligonucleotide is WV-31563. In some embodiments, a single oligonucleotide is WV-31564. In some embodiments, a chirally controlled oligonucleotide composition is a pharmaceutical composition comprising a therapeutically effective amount of a single oligonucleotide which may exist in various forms (e.g., an acid form, and/or one or more pharmaceutically acceptable salt forms). In some embodiments, a pharmaceutical composition may additionally comprise a pharmaceutically acceptable carrier and other components as described herein. In some embodiments, a pharmaceutical composition is a liquid composition, e.g., a buffer solution having a suitable pH (e.g., about 7, about 7-8, about 7.4, etc.), which comprises one or more dissolved oligonucleotides.

In some embodiments, such a provided oligonucleotide composition may be chirally controlled, and comprises a plurality of the oligonucleotides, wherein one or more (e.g., 1, 2, 3, 4, 5, 6, 7, 8, 9, 10, 11, 12, 13, 14, 15, 16, 17, 18, 19, 20 or more) internucleotidic linkages are chirally controlled. In some embodiments, each chiral internucleotidic linkage is independently chirally controlled. In some embodiments, a chirally controlled internucleotidic linkage is one that of S, R, nR or nS as indicated in “Linkage/Stereochemistry” in Table A1.

In some embodiments, the present disclosure provides a chirally controlled DMD oligonucleotide composition, wherein the DMD oligonucleotide is capable of mediating skipping of a DMD exon and the DMD oligonucleotide is

In some embodiments, the present disclosure provides a chirally controlled DMD oligonucleotide composition, wherein the DMD oligonucleotide is capable of mediating skipping of DMD exon 51 and the DMD oligonucleotide is WV-20011.

In some embodiments, the present disclosure provides a chirally controlled DMD oligonucleotide composition, wherein the DMD oligonucleotide is capable of mediating skipping of DMD exon 51 and the DMD oligonucleotide is WV-20052.

In some embodiments, the present disclosure provides a chirally controlled DMD oligonucleotide composition, wherein the DMD oligonucleotide is capable of mediating skipping of DMD exon 51 and the DMD oligonucleotide is WV-20059.

In some embodiments, the present disclosure provides a chirally controlled DMD oligonucleotide composition, wherein the DMD oligonucleotide is capable of mediating skipping of DMD exon 51 and the DMD oligonucleotide is WV-20072.

In some embodiments, the present disclosure provides a chirally controlled DMD oligonucleotide composition, wherein the DMD oligonucleotide is capable of mediating skipping of DMD exon 51 and the DMD oligonucleotide is WV-20073.

In some embodiments, the present disclosure provides a chirally controlled DMD oligonucleotide composition, wherein the DMD oligonucleotide is capable of mediating skipping of DMD exon 51 and the DMD oligonucleotide is WV-20074.

In some embodiments, the present disclosure provides a chirally controlled DMD oligonucleotide composition, wherein the DMD oligonucleotide is capable of mediating skipping of DMD exon 51 and the DMD oligonucleotide is WV-20075.

In some embodiments, the present disclosure provides a chirally controlled DMD oligonucleotide composition, wherein the DMD oligonucleotide is capable of mediating skipping of DMD exon 51 and the DMD oligonucleotide is WV-20076.

In some embodiments, the present disclosure provides a chirally controlled DMD oligonucleotide composition, wherein the DMD oligonucleotide is capable of mediating skipping of DMD exon 51 and the DMD oligonucleotide is WV-20096.

In some embodiments, the present disclosure provides a chirally controlled DMD oligonucleotide composition, wherein the DMD oligonucleotide is capable of mediating skipping of DMD exon 51 and the DMD oligonucleotide is WV-20097.

In some embodiments, the present disclosure provides a chirally controlled DMD oligonucleotide composition, wherein the DMD oligonucleotide is capable of mediating skipping of DMD exon 51 and the DMD oligonucleotide is WV-20101.

In some embodiments, the present disclosure provides a chirally controlled DMD oligonucleotide composition, wherein the DMD oligonucleotide is capable of mediating skipping of DMD exon 51 and the DMD oligonucleotide is WV-20119.

In some experiments, provided DMD oligonucleotides can provide surprisingly high skipping of exon 51, e.g., when compared to those of Drisapersen and/or Eteplirsen. For example, various chirally controlled DMD oligonucleotide compositions each showed a superior capability, in some embodiments many fold higher, to mediate skipping of exon 51 in dystrophin, compared to Drisapersen and/or Eteplirsen. Certain data are provided in the present disclosure as examples.

In some embodiments, when assaying example DMD oligonucleotides in mice, DMD oligonucleotides are intravenous injected via tail vein in male C57BL/10ScSndmdmdx mice (4-5 weeks old), at tested amounts, e.g., 10 mg/kg, 30 mg/kg, etc. In some embodiments, tissues are harvested at tested times, e.g., on Day, e.g., 2, 7 and/or 14, etc., after injection, in some embodiments, fresh-frozen in liquid nitrogen and stored in −80° C. until analysis.

Various assays can be used to assess DMD oligonucleotide levels in accordance with the present disclosure. In some embodiments, hybrid-ELISA is used to quantify DMD oligonucleotide levels in tissues using test article serial dilution as standard curve: for example, in an example procedure, maleic anhydride activated 96-well plate (Pierce 15110) was coated with 50 pd of capture probe at 500 nM in 2.5% NaHCO3 (Gibco, 25080-094) for 2 hours at 37° C. The plate was then washed 3 times with PBST (PBS+0.1% Tween-20), and blocked with 5% fat free milk-PBST at 37° C. for 1 hour. Test article DMD oligonucleotide was serial diluted into matrix. This standard together with original samples were diluted with lysis buffer (4 M Guanidine; 0.33% N-Lauryl Sarcosine: 25 mM Sodium Citrate; 10 mM DTI) so that DMD oligonucleotide amount in all samples is less than 100 ng/mL. 20 μl of diluted samples were mixed with 180 μl of 333 nM detection probe diluted in PBST, then denatured in PCR machine (65° C., 10 min, 95° C., 15 min, 4° C. ∞). 50 μl of denatured samples were distributed in blocked ELISA plate in triplicates, and incubated overnight at 4° C. After 3 washes of PBST, 1:2000 streptavidin-AP in PBST was added, 50 μl per well and incubated at room temperature for 1 hour. After extensive wash with PBST, 100 μl of AttoPhos (Promega S1000) was added, incubated at room temperature in dark for 10 min and read on plate reader (Molecular Device, M5) fluorescence channel: Ex435 nm, Em555 nm. Oligonucleotides in samples were calculated according to standard curve by 4-parameter regression.

In some embodiments, provided DMD oligonucleotides are stable in both plasma and tissue homogenates.

### Example Dystrophin Oligonucleotides and Compositions for Exon Skipping of Exon 51

In some embodiments, the present disclosure provides DMD oligonucleotides, DMD oligonucleotide compositions, and methods of use thereof for mediating skipping of exon 51 in DMD (e.g., of mouse, human, etc.).

In some embodiments, a provided DMD oligonucleotide and/or composition is capable of mediating skipping of exon 51.

In some embodiments, non-limiting examples of such DMD oligonucleotides and compositions include those of: WV-20011, WV-20052, WV-20059, WV-20072, WV-20073, WV-20074, WV-20075, WV-20076, WV-20096, WV-20097, WV-20101, and WV-20119, and other DMD oligonucleotides having a base sequence which comprises at least 15 contiguous bases of any of these DMD oligonucleotides.

In some embodiments, the sequence of the region of interest for exon 51 skipping differs between the mouse and human.

Various assays can be utilized to assess DMD oligonucleotides for exon skipping in accordance with the present disclosure. In some embodiments, in order to test the efficacy of a particular combination of chemistry and stereochemistry of a DMD oligonucleotide intended for exon 51 skipping in human, a corresponding DMD oligonucleotide can be prepared which has the mouse sequence, and then tested in mouse. The present disclosure recognizes that in the human and mouse homologs of exon 51, a few differences exist (underlined below) (SEQ ID NOS: 402 and 403):

M
GTGGTTACTAAGGAAACTGTCATCTCCAAACTAGAAATGCCATCTTCT
TTGCTGTTGGAG
H
GTGGTTACTAAGGAAACTGCCATCTCCAAACTAGAAATGCCATCTTCC
TTGATGTTGGAG

where M is Mouse, nt 7571-7630; and H is Human, nt 7665-7724.

Because of these differences, slightly different DMD oligonucleotides for skipping exon 51 can be prepared for testing in mouse and human. As a non-limiting example, the following DMD oligonucleotide sequences can be used for testing in human and mouse (SEQ ID NOS: 404 and 405):

HUMAN DMD oligonucleotide sequence:
UCAAGGAAGAUGGCAUUUCU
MOUSE DMD oligonucleotide sequence:
GCAAAGAAGAUGGCAUUUCU

Mismatches between human and mouse are underlined.

A DMD oligonucleotide intended for treating a human subject can be constructed with a particular combination of base sequence (e.g., 1 UCAAGGAAGAUGGCAUUUCU) (SEQ ID NO: 404), and a particular pattern of chemistry, internucleotidic linkages, stereochemistry, and additional chemical moieties (if any). Such a DMD oligonucleotide can be tested in vitro in human cells or in vivo in human subjects, but may have limited suitability for testing in mouse, for example, because base sequences of the two have mismatches.

A corresponding DMD oligonucleotide can be constructed with the corresponding mouse base sequence (GCAAAGAAGAUGGCAUUUCU) (SEQ ID NO: 405) and the same pattern of chemistry, internucleotidic linkages, stereochemistry, and additional chemical moieties (if any). Such a DMD oligonucleotide can be tested in vivo in mouse. Several DMD oligonucleotides comprising the mouse base sequence were constructed and tested.

In some embodiments, a human DMD exon skipping DMD oligonucleotide can be tested in a mouse which has been modified to comprise a DMD gene comprising the human sequence.

Various DMD oligonucleotides comprising various patterns of modifications are described herein. The Tables below show test results of certain DMD oligonucleotides. Generally, numbers indicate the amount of skipping, wherein 100 would indicate 100% skipping and 0 would indicate no skipping, unless otherwise indicated. To assay exon skipping of DMD, DMD oligonucleotides were tested in vitro in Δ52 human patient-derived myoblast cells and/or Δ45-52 human patient-derived myoblast cells (human cells wherein the exon 52 or exons 45-52 were already deleted). Unless noted otherwise, in various experiments, DMD oligonucleotides were delivered gymnotically.

TABLE 1
Activity of certain DMD oligonucleotides
Activity of various DMD exon 51 DMD oligonucleotides was tested in vitro.
Numbers indicate amount of skipping DMD exon 51 (as a percentage
of total DMD mRNA, where 100 would represent 100% skipped).
Amounts tested were: 10, 3.3 and 1.1 uM.

| Conc. | 10 | 3.3 | 1.1 | Conc. | 10 | 3.3 | 1.1 |
|---|---|---|---|---|---|---|---|
| WV- | 20.8 | 9 | 4.1 | WV- | 36.9 | 10.4 | 4.7 |
| 3152 | 22 | 10 | 4.9 | 14522 | 27.4 | 10.4 | 4.2 |
| | 17.3 | 9.3 | 3.2 | | 21 | 12.6 | 5.6 |
| | 21.3 | 7.2 | 4.4 | | 26.5 | 10.4 | 5.7 |
| WV- | 27.4 | 13.2 | 12.7 | WV- | 27.2 | 8.1 | 6.2 |
| 15860 | 30.4 | 15.4 | 9 | 14523 | 28.3 | 8.5 | 4.9 |
| | 33 | 14.2 | 6 | | 18.4 | 9.1 | 3.6 |
| | 33.4 | 16.9 | 5.9 | | 18.7 | 9.6 | 4.4 |
| WV- | 26.6 | 9.2 | 5.6 | Mock | 0.21 | | |
| 15861 | 28.5 | 6.1 | 5.4 | | 0.35 | | |
| | 34.1 | 8.2 | 5.2 | | 0.48 | | |
| | 29.9 | 11.1 | 4 | | 0.24 | | |
| WV- | 30.7 | | 7.8 | | | | |
| 15862 | 33.3 | | 7.2 | | | | |
| | 21.9 | 15.1 | 6.8 | | | | |
| | 26.4 | 13.2 | 7.2 | | | | |

TABLE 2
Activity of certain DMD oligonucleotides
Oligonucleotides for skipping DMD exon 51 were tested in vitro.
Numbers indicate amount of skipping DMD exon 51 (as a percentage of
total DMD mRNA, where 100 would represent 100% skipped).
Concentrations of DMD oligonucleotides used: 10, 3.3 and 1.1 uM.

| | 10 uM | 3.3 uM | 1.1 uM | | 10 uM | 3.3 uM | 1.1 uM |
|---|---|---|---|---|---|---|---|
| Mock | 0.2 | 0.3 | 0.2 | WV- | 37.6 | 22.6 | 9 |
| | 0.3 | 0.2 | 0.3 | 17861 | 38.8 | 22.5 | 8.9 |
| | 0.2 | 0 | 0.2 | | 40.7 | 24.4 | 13.2 |
| | 0.2 | 0.6 | 0.2 | | 41.7 | 25.4 | 11.6 |
| WV- | 3.1 | 1.6 | 0.7 | WV- | 38.4 | 18.9 | 8.1 |
| 7336 | 8.9 | 1.8 | 0.1 | 17862 | 34.1 | 19.6 | 9 |
| | 5.4 | 1.4 | 0.9 | | 34.8 | 26 | 10 |
| | 4.9 | 1.5 | 0.7 | | 36.1 | 21.4 | 9.5 |
| WV- | 32.4 | 26.5 | 7.5 | WV- | 32.7 | 18.2 | 9.2 |
| 3152 | 27.2 | 22.2 | 8.4 | 17863 | 35.1 | 18.9 | 9.3 |
| | 28 | 14.5 | 7.6 | | 34.8 | 18.2 | 8.6 |
| | 26.8 | 14.8 | 7.3 | | 30.7 | 17 | 9 |
| WV- | 43.3 | 25.7 | 10.2 | WV- | 37.3 | 23.6 | 11.7 |
| 15860 | 37.9 | 23.8 | 9.6 | 17864 | 41.4 | 23.3 | 10.6 |
| | 38.4 | 24.5 | 11.2 | | 39.9 | 20.6 | 17.5 |
| | 42.4 | 21.9 | 11 | | 38.8 | 21.7 | 10.2 |
| WV- | 42.3 | 26.7 | 16.3 | WV- | 35.9 | 16.5 | 9.3 |
| 17859 | 41.3 | 26 | 16.8 | 17865 | 34 | 16.7 | 7.5 |
| | 39.9 | 22.9 | 15.5 | | 34.4 | 17.5 | 11.9 |
| | 48.6 | 23.6 | 14.9 | | 34.1 | 17.8 | 9.8 |
| WV- | 38.1 | 19.3 | 11.7 | WV- | 48.7 | 28.4 | 17.7 |
| 17860 | 35.3 | 19.2 | 12 | 17866 | 43.3 | 28.6 | 13.1 |
| | 41 | 28.2 | 16.4 | | 44.5 | 24.8 | 15.4 |
| | 40.4 | 21.9 | 11.1 | | 45.1 | 30.5 | 16.3 |

TABLE 3
Activity of certain DMD oligonucleotides
Oligonucleotides for skipping DMD exon 51 were tested in vitro.
Numbers indicate amount of skipping DMD exon 51
(as a percentage of total DMD mRNA,
where 100 would represent 100% skipped).
Concentrations of DMD oligonucleotides used: 10 and 3.3 uM.

| | 10 uM | 3.3 uM | | 10 uM | 3.3 uM |
|---|---|---|---|---|---|
| Mock | 0 | 0 | WV- | 14.6 | 4.8 |
| | 0 | 0 | 20058 | 12 | 3.7 |
| | 0 | 0 | | 12.6 | 3.5 |
| | 0 | 0 | | | |
| WV- | 15.9 | 7 | WV- | 35.8 | 26.5 |
| 20034 | 17.1 | 8.4 | 20061 | 39.3 | 24.2 |
| | 16.1 | 7.3 | | 39.9 | 22.8 |
| | 15.3 | 7.2 | | | |
| WV- | 29.7 | 18.3 | WV- | 26.5 | 17.6 |
| 20037 | 27.2 | 17.5 | 20064 | 24.5 | 16.4 |
| | 26.6 | 19.4 | | 27.5 | 17.1 |
| | 29.2 | 18.4 | | | |
| WV- | 9.6 | 4.9 | WV- | 15.7 | 8.3 |
| 20040 | 9.1 | 5.2 | 20067 | 16.8 | 9.3 |
| | 11.4 | 3.5 | | 17.3 | 8.6 |
| | 10.9 | 2.9 | | 16.3 | 8.7 |
| WV- | 20.2 | 9.6 | WV- | 41.3 | 26.4 |
| 20043 | 20.4 | 9.8 | 20070 | 31.7 | 22.3 |
| | 18.9 | 9.8 | | 39.7 | 27.2 |
| | 21 | 10.4 | | 38.4 | 26.9 |
| WV- | 28.5 | 14.7 | WV- | 30.9 | 21.1 |
| 20046 | 29.8 | 14.2 | 20073 | 26.9 | 17.9 |
| | 29.2 | 15.8 | | 31.1 | 20.2 |
| | 26.6 | 14.5 | | 30.7 | 22.2 |
| WV- | 20.9 | 11.6 | WV- | 23.2 | 16.8 |
| 20049 | 18.6 | 12.2 | 20076 | 18.9 | 11.4 |
| | 18.4 | 11.7 | | 21.8 | 16.9 |
| | | | | 22.8 | 15.8 |
| WV- | 28.8 | 18.8 | WV- | 35.7 | 24.8 |
| 20052 | 30.1 | 18.6 | 3152 | 33.5 | 24.9 |
| | 29.6 | 20.1 | | 32.1 | 25.3 |
| WV- | 26.8 | 17 | WV- | 41.9 | 27.5 |
| 20055 | 25.3 | 16.6 | 15860 | 43.6 | 30.7 |
| | 24.1 | 17 | | 42.4 | 30 |

TABLE 4A
Activity of certain DMD oligonucleotides
Oligonucleotides for skipping DMD exon 51 were tested in vitro.
Oligonucleotides were dosed 4 d at 10 uM.
Numbers indicate amount of skipping DMD exon 51 (as a percentage
of total DMD mRNA, where 100 would represent 100% skipped).

| | | | | | | | | | |
|---|---|---|---|---|---|---|---|---|---|
| WV-3152 | 19 | 20 | 12 | 14 | WV-20093 | 35 | 34 | 35 | 38 |
| WV-15860 | 29 | 31 | 26 | 23 | WV-20092 | 25 | 26 | 25 | 25 |
| WV-20140 | 1 | 1 | 1 | 1 | WV-20091 | 28 | 27 | 30 | 32 |
| WV-20139 | 3 | 3 | 2 | 2 | WV-20090 | 21 | 19 | 22 | 22 |
| WV-20138 | | | 2 | 3 | WV-20089 | 8 | 7 | 8 | 9 |
| WV-20137 | 4 | 5 | | | WV-20088 | 22 | 21 | 26 | 25 |
| WV-20136 | | | | | WV-20087 | 28 | 28 | 33 | 32 |
| WV-20135 | 5 | 5 | 5 | 5 | WV-20086 | 25 | 25 | 27 | 26 |
| WV-20134 | 5 | 6 | 5 | 4 | WV-20085 | 33 | 31 | 30 | 31 |
| WV-20133 | 17 | 17 | 13 | 13 | WV-20084 | 21 | 22 | 21 | 21 |
| WV-20132 | 8 | 8 | 6 | 6 | WV-20083 | 21 | 21 | 19 | 17 |
| WV-20131 | 14 | 16 | 12 | 12 | WV-20082 | 42 | 37 | 32 | 30 |
| WV-20130 | 10 | 9 | 8 | 8 | WV-20081 | 41 | 41 | 30 | 30 |
| WV-20129 | 12 | 14 | 11 | 11 | WV-20080 | 49 | 44 | 26 | 25 |
| WV-20128 | 9 | 9 | 8 | 8 | WV-20079 | 42 | 38 | 53 | 51 |
| WV-20127 | | | 8 | 8 | WV-20078 | 27 | 28 | 36 | 35 |
| WV-20126 | 7 | 8 | 8 | 7 | WV-20077 | 10 | 10 | 10 | 10 |
| WV-20125 | 8 | 8 | 8 | 8 | WV-20076 | 45 | 45 | 45 | 41 |
| WV-20124 | 22 | 21 | 21 | 21 | WV-20075 | 40 | 31 | 37 | 42 |
| WV-20123 | 13 | 13 | 14 | 12 | WV-20074 | 55 | 57 | 53 | 56 |
| WV-20122 | 11 | 12 | 12 | 11 | WV-20073 | 51 | 55 | 51 | 50 |
| WV-20121 | 21 | 22 | 22 | 21 | WV-20072 | 41 | 36 | 37 | 36 |
| WV-20120 | 28 | 30 | 32 | 33 | WV-20071 | 42 | 40 | 44 | 46 |
| WV-20119 | 52 | 50 | | | WV-20070 | 18 | 18 | 25 | 25 |
| WV-20118 | 39 | 37 | 27 | 26 | WV-20069 | 11 | 11 | 10 | 9 |
| WV-20117 | 18 | 17 | 15 | 18 | WV-20068 | 20 | 17 | 20 | 18 |
| WV-20116 | 20 | 20 | 17 | 17 | WV-20067 | 12 | 9 | 11 | 11 |
| WV-20115 | 8 | 8 | 8 | 6 | WV-20066 | 12 | 11 | 13 | 12 |
| WV-20114 | 19 | 20 | 15 | 14 | WV-20065 | 16 | 15 | 16 | 14 |
| WV-20113 | 20 | 18 | 17 | 15 | WV-20064 | 37 | 35 | 37 | 36 |
| WV-20112 | 16 | 15 | 12 | 12 | WV-20063 | 19 | | 24 | 22 |
| WV-20111 | 31 | 30 | 33 | 31 | WV-20062 | 6 | 6 | 7 | 7 |
| WV-20110 | 14 | 14 | 14 | 12 | WV-20061 | 24 | 23 | 26 | 24 |
| WV-20109 | 20 | 21 | 25 | 24 | WV-20060 | 16 | 17 | 16 | 17 |
| WV-20108 | 27 | 25 | 22 | 22 | WV-20059 | 55 | 42 | 62 | 67 |
| WV-20107 | 20 | 19 | 16 | 14 | WV-20058 | 28 | 30 | 33 | 33 |
| WV-20106 | 44 | 42 | 34 | 37 | WV-20057 | 37 | 38 | 37 | 34 |
| WV-20105 | 23 | 22 | 18 | 18 | WV-20056 | 35 | 34 | 33 | 35 |
| WV-20104 | 41 | 40 | 33 | 28 | WV-20055 | | | 40 | 40 |
| WV-20103 | 48 | 52 | 53 | 53 | WV-20054 | 25 | 25 | 35 | 36 |
| WV-20102 | 54 | 52 | 55 | 59 | WV-20053 | 43 | 45 | 46 | 46 |
| WV-20101 | 38 | 39 | 38 | 43 | WV-20052 | 47 | 47 | 53 | 46 |
| WV-20100 | 52 | 51 | 48 | 50 | WV-20051 | 30 | 33 | 30 | 30 |
| WV-20099 | 53 | 51 | 47 | 48 | WV-20050 | 29 | 28 | 28 | 26 |
| WV-20098 | 46 | 44 | 45 | 46 | WV-20049 | 41 | 41 | 38 | 38 |
| WV-20097 | 47 | 46 | 51 | 48 | WV-20049 | 24 | 23 | 22 | 21 |
| WV-20096 | 45 | 41 | 42 | 43 | | | | | |
| WV-20095 | 43 | 41 | 50 | 47 | | | | | |
| WV-20094 | 55 | 50 | 57 | 55 | | | | | |

Various oligonucleotides which had been shown to induce skipping of exon 51 in DMD transcripts were further tested for their ability to facilitate production of corresponding internally truncated DMD protein. Experiments measured production of a protein which was recognized by anti-Dystrophin antibody (Abcam, Cambridge, MA) and which was of a size corresponding to that which would be theoretically produced by transcription of a DMD transcript in which exon 51 was skipped. Experiments were performed in vitro in delta48-50 cells, treated gymnotically with 5 uM of oligonucleotide, and 7 day treatment. Oligonucleotide WV-3152 (at 5 uM) produced 18% internally-truncated DMD protein, normalized to the wild-type dystrophin level observed in wild-type (healthy) human immortalized myoblasts; and WV-15860 (5 uM), 31%.

TABLE 4B
Activity of certain DMD oligonucleotides Patient Δ48-50 cells
were dosed for 4d with oligonucleotides in differentiation
media. RNA was harvested by Trizol extraction.
TaqMan signal was normalized to SFSR9 internal control.
Numbers indicate amount of skipping DMD exon 51
(as a percentage of total DMD mRNA, where 100
would represent 100% skipped).

| | 10 uM | 3.3 uM | | 10 uM | 3.3 uM |
|---|---|---|---|---|---|
| Mock | 0 | 0 | WV- | 14.6 | 4.8 |
| | 0 | 0 | 20058 | 12 | 3.7 |
| | 0 | 0 | | 12.6 | 3.5 |
| | 0 | 0 | | | |
| WV- | 15.9 | 7 | WV- | 35.8 | 26.5 |
| 20034 | 17.1 | 8.4 | 20061 | 39.3 | 24.2 |
| | 16.1 | 7.3 | | 39.9 | 22.8 |
| | 15.3 | 7.2 | | | |
| WV- | 29.7 | 18.3 | WV- | 26.5 | 17.6 |
| 20037 | 27.2 | 17.5 | 20064 | 24.5 | 16.4 |
| | 26.6 | 19.4 | | 27.5 | 17.1 |
| | 29.2 | 18.4 | | | |
| WV- | 9.6 | 4.9 | WV- | 15.7 | 8.3 |
| 20040 | 9.1 | 5.2 | 20067 | 16.8 | 9.3 |
| | 11.4 | 3.5 | | 17.3 | 8.6 |
| | 10.9 | 2.9 | | 16.3 | 8.7 |
| WV- | 20.2 | 9.6 | WV- | 41.3 | 26.4 |
| 20043 | 20.4 | 9.8 | 20070 | 31.7 | 22.3 |
| | 18.9 | 9.8 | | 39.7 | 27.2 |
| | 21 | 10.4 | | 38.4 | 26.9 |
| WV- | 28.5 | 14.7 | WV- | 30.9 | 21.1 |
| 20046 | 29.8 | 14.2 | 20073 | 26.9 | 17.9 |
| | 29.2 | 15.8 | | 31.1 | 20.2 |
| | 26.6 | 14.5 | | 30.7 | 22.2 |
| WV- | 20.9 | 11.6 | WV- | 23.2 | 16.8 |
| 20049 | 18.6 | 12.2 | 20076 | 18.9 | 11.4 |
| | 18.4 | 11.7 | | 21.8 | 16.9 |
| | | | | 22.8 | 15.8 |
| WV- | 28.8 | 18.8 | WV- | 35.7 | 24.8 |
| 20052 | 30.1 | 18.6 | 3152 | 33.5 | 24.9 |
| | 29.6 | 20.1 | | 32.1 | 25.3 |
| WV- | 26.8 | 17 | WV- | 41.9 | 27.5 |
| 20055 | 25.3 | 16.6 | 15860 | 43.6 | 30.7 |
| | 24.1 | 17 | | 42.4 | 30 |

TABLE 4C
Activity of certain DMD oligonucleotides Patient-derived
Δ48-50 cells were dosed with oligonucleotide in
differentiation media under free-uptake conditions for 4 days.
RNA harvested by Trizol extraction. TaqMan signal for DMD ‘skipped’
and DMD ‘total’ transcripts were normalized to SFSR9 internal control.
Numbers indicate amount of skipping DMD exon 51 (as a percentage
of total DMD mRNA, where 100 would represent 100% skipped).

| | 10 uM | 3.3 uM | 1.1 uM |
|---|---|---|---|
| Mock | 0.2 | 0.3 | 0.2 |
| | 0.3 | 0.2 | 0.3 |
| | 0.2 | 0 | 0.2 |
| | 0.2 | 0.6 | 0.2 |
| WV- | 3.1 | 1.6 | 0.7 |
| 7336 | 8.9 | 1.8 | 0.1 |
| | 5.4 | 1.4 | 0.9 |
| | 4.9 | 1.5 | 0.7 |
| WV- | 32.4 | 26.5 | 7.5 |
| 3152 | 27.2 | 22.2 | 8.4 |
| | 28 | 14.5 | 7.6 |
| | 26.8 | 14.8 | 7.3 |
| WV- | 43.3 | 25.7 | 10.2 |
| 15860 | 37.9 | 23.8 | 9.6 |
| | 38.4 | 24.5 | 11.2 |
| | 42.4 | 21.9 | 11 |
| WV- | 42.3 | 26.7 | 16.3 |
| 17859 | 41.3 | 26 | 16.8 |
| | 39.9 | 22.9 | 15.5 |
| | 48.6 | 23.6 | 14.9 |
| WV- | 38.1 | 19.3 | 11.7 |
| 17860 | 35.3 | 19.2 | 12 |
| | 41 | 28.2 | 16.4 |
| | 40.4 | 21.9 | 11.1 |
| WV- | 37.6 | 22.6 | 9 |
| 17861 | 38.8 | 22.5 | 8.9 |
| | 40.7 | 24.4 | 13.2 |
| | 41.7 | 25.4 | 11.6 |
| WV- | 38.4 | 18.9 | 8.1 |
| 17862 | 34.1 | 19.6 | 9 |
| | 34.8 | 26 | 10 |
| | 36.1 | 21.4 | 9.5 |
| WV- | 32.7 | 18.2 | 9.2 |
| 17863 | 35.1 | 18.9 | 9.3 |
| | 34.8 | 18.2 | 8.6 |
| | 30.7 | 17 | 9 |
| WV- | 37.3 | 23.6 | 11.7 |
| 17864 | 41.4 | 23.3 | 10.6 |
| | 39.9 | 20.6 | 17.5 |
| | 38.8 | 21.7 | 10.2 |
| WV- | 35.9 | 16.5 | 9.3 |
| 17865 | 34 | 16.7 | 7.5 |
| | 34.4 | 17.5 | 11.9 |
| | 34.1 | 17.8 | 9.8 |
| WV- | 48.7 | 28.4 | 17.7 |
| 17866 | 43.3 | 28.6 | 13.1 |
| | 44.5 | 24.8 | 15.4 |
| | 45.1 | 30.5 | 16.3 |

TABLE 4D
Activity of certain DMD oligonucleotides Patient-derived Δ48-50 cells
were dosed with oligonucleotide in differentiation media under
free-uptake conditions for 4 days. RNA from 24WP harvested by
bead-based extraction. TaqMan signal for DMD
‘skipped’ and DMD ‘total’ transcripts were normalized to SFSR9
internal control. Numbers indicate amount of skipping
DMD exon 51 (as a percentage of total DMD mRNA,
where 100 would represent 100% skipped).

| | 10 uM | 3.3 uM | | 10 uM | 3.3 uM |
|---|---|---|---|---|---|
| Mock | 0 | 0 | WV- | 14.6 | 4.8 |
| | 0 | 0 | 20058 | 12 | 3.7 |
| | 0 | 0 | | 12.6 | 3.5 |
| | 0 | 0 | | | |
| WV- | 15.9 | 7 | WV- | 35.8 | 26.5 |
| 20034 | 17.1 | 8.4 | 20061 | | |
| | 16.1 | 7.3 | | 39.3 | 24.2 |
| | 15.3 | 7.2 | | 39.9 | 22.8 |
| WV- | 29.7 | 18.3 | WV- | 26.5 | 17.6 |
| 20037 | 27.2 | 17.5 | 20064 | 24.5 | 16.4 |
| | 26.6 | 19.4 | | 27.5 | 17.1 |
| | 29.2 | 18.4 | | | |
| WV- | 9.6 | 4.9 | WV- | 15.7 | 8.3 |
| 20040 | 9.1 | 5.2 | 20067 | 16.8 | 9.3 |
| | 11.4 | 3.5 | | 17.3 | 8.6 |
| | 10.9 | 2.9 | | 16.3 | 8.7 |
| WV- | 20.2 | 9.6 | WV- | 41.3 | 26.4 |
| 20043 | 20.4 | 9.8 | 20070 | 31.7 | 22.3 |
| | 18.9 | 9.8 | | 39.7 | 27.2 |
| | 21 | 10.4 | | 38.4 | 26.9 |
| WV- | 28.5 | 14.7 | WV- | 30.9 | 21.1 |
| 20046 | 29.8 | 14.2 | 20073 | 26.9 | 17.9 |
| | 29.2 | 15.8 | | 31.1 | 20.2 |
| | 26.6 | 14.5 | | 30.7 | 22.2 |
| WV- | 20.9 | 11.6 | WV- | 23.2 | 16.8 |
| 20049 | | | 20076 | 18.9 | 11.4 |
| | 18.6 | 12.2 | | 21.8 | 16.9 |
| | 18.4 | 11.7 | | 22.8 | 15.8 |
| WV- | 28.8 | 18.8 | WV- | 35.7 | 24.8 |
| 20052 | 30.1 | 18.6 | 3152 | 33.5 | 24.9 |
| | 29.6 | 20.1 | | 32.1 | 25.3 |
| WV- | 26.8 | 17 | WV- | 41.9 | 27.5 |
| 20055 | 25.3 | 16.6 | 15860 | 43.6 | 30.7 |
| | 24.1 | 17 | | 42.4 | 30 |

TABLE 4E
Activity of certain DMD oligonucleotides
Conditions and parameters: Δ48-50 cells (Delta 48-50) (no pre-differentiation) in
96 WP in biological duplicate at 10 uM for 4 days
Samples were lysed and baked in bead lysis buffer, frozen at −80
Bead-based extraction with manual (vs Bravo) washes
Numbers indicate amount of skipping DMD exon 51 (as a percentage of total
DMD mRNA, where 100 would represent 100% skipped).

| | | | | | | | | | |
|---|---|---|---|---|---|---|---|---|---|
| WV-3152 | 19 | 20 | 12 | 14 | WV-20094 | 55 | 50 | 57 | 55 |
| WV-15860 | 29 | 31 | 26 | 23 | WV-20093 | 35 | 34 | 35 | 38 |
| WV-20140 | 1 | 1 | 1 | 1 | WV-20092 | 25 | 26 | 25 | 25 |
| WV-20139 | 3 | 3 | 2 | 2 | WV-20091 | 28 | 27 | 30 | 32 |
| WV-20138 | | | 2 | 3 | WV-20090 | 21 | 19 | 22 | 22 |
| WV-20137 | 4 | 5 | | | WV-20089 | 8 | 7 | 8 | 9 |
| WV-20136 | | | | | WV-20088 | 22 | 21 | 26 | 25 |
| WV-20135 | 5 | 5 | 5 | 5 | WV-20087 | 28 | 28 | 33 | 32 |
| WV-20134 | 5 | 6 | 5 | 4 | WV-20086 | 25 | 25 | 27 | 26 |
| WV-20133 | 17 | 17 | 13 | 13 | WV-20085 | 33 | 31 | 30 | 31 |
| WV-20132 | 8 | 8 | 6 | 6 | WV-20084 | 21 | 22 | 21 | 21 |
| WV-20131 | 14 | 16 | 12 | 12 | WV-20083 | 21 | 21 | 19 | 17 |
| WV-20130 | 10 | 9 | 8 | 8 | WV-20082 | 42 | 37 | 32 | 30 |
| WV-20129 | 12 | 14 | 11 | 11 | WV-20081 | 41 | 41 | 30 | 30 |
| WV-20128 | 9 | 9 | 8 | 8 | WV-20080 | 49 | 44 | 26 | 25 |
| WV-20127 | | | 8 | 8 | WV-20079 | 42 | 38 | 53 | 51 |
| WV-20126 | 7 | 8 | 8 | 7 | WV-20078 | 27 | 28 | 36 | 35 |
| WV-20125 | 8 | 8 | 8 | 8 | WV-20077 | 10 | 10 | 10 | 10 |
| WV-20124 | 22 | 21 | 21 | 21 | WV-20076 | 45 | 45 | 45 | 41 |
| WV-20123 | 13 | 13 | 14 | 12 | WV-20075 | 40 | 31 | 37 | 42 |
| WV-20122 | 11 | 12 | 12 | 11 | WV-20074 | 55 | 57 | 53 | 56 |
| WV-20121 | 21 | 22 | 22 | 21 | WV-20073 | 51 | 55 | 51 | 50 |
| WV-20120 | 28 | 30 | 32 | 33 | WV-20072 | 41 | 36 | 37 | 36 |
| WV-20119 | 52 | 50 | | | WV-20071 | 42 | 40 | 44 | 46 |
| WV-20118 | 39 | 37 | 27 | 26 | WV-20070 | 18 | 18 | 25 | 25 |
| WV-20117 | 18 | 17 | 15 | 18 | WV-20069 | 11 | 11 | 10 | 9 |
| WV-20116 | 20 | 20 | 17 | 17 | WV-20068 | 20 | 17 | 20 | 18 |
| WV-20115 | 8 | 8 | 8 | 6 | WV-20067 | 12 | 9 | 11 | 11 |
| WV-20114 | 19 | 20 | 15 | 14 | WV-20066 | 12 | 11 | 13 | 12 |
| WV-20113 | 20 | 18 | 17 | 15 | WV-20065 | 16 | 15 | 16 | 14 |
| WV-20112 | 16 | 15 | 12 | 12 | WV-20064 | 37 | 35 | 37 | 36 |
| WV-20111 | 31 | 30 | 33 | 31 | WV-20063 | 19 | | 24 | 22 |
| WV-20110 | 14 | 14 | 14 | 12 | WV-20062 | 6 | 6 | 7 | 7 |
| WV-20109 | 20 | 21 | 25 | 24 | WV-20061 | 24 | 23 | 26 | 24 |
| WV-20108 | 27 | 25 | 22 | 22 | WV-20060 | 16 | 17 | 16 | 17 |
| WV-20107 | 20 | 19 | 16 | 14 | WV-20059 | 55 | 42 | 62 | 67 |
| WV-20106 | 44 | 42 | 34 | 37 | WV-20058 | 28 | 30 | 33 | 33 |
| WV-20105 | 23 | 22 | 18 | 18 | WV-20057 | 37 | 38 | 37 | 34 |
| WV-20104 | 41 | 40 | 33 | 28 | WV-20056 | 35 | 34 | 33 | 35 |
| WV-20103 | 48 | 52 | 53 | 53 | WV-20055 | | | 40 | 40 |
| WV-20102 | 54 | 52 | 55 | 59 | WV-20054 | 25 | 25 | 35 | 36 |
| WV-20101 | 38 | 39 | 38 | 43 | WV-20053 | 43 | 45 | 46 | 46 |
| WV-20100 | 52 | 51 | 48 | 50 | WV-20052 | 47 | 47 | 53 | 46 |
| WV-20099 | 53 | 51 | 47 | 48 | WV-20051 | 30 | 33 | 30 | 30 |
| WV-20098 | 46 | 44 | 45 | 46 | WV-20050 | 29 | 28 | 28 | 26 |
| WV-20097 | 47 | 46 | 51 | 48 | WV-20049 | 41 | 41 | 38 | 38 |
| WV-20096 | 45 | 41 | 42 | 43 | WV-20049 | 24 | 23 | 22 | 21 |
| WV-20095 | 43 | 41 | 50 | 47 | | | | | |

TABLE 4F
Activity of certain DMD oligonucleotides
Delta 48-50 cells were treated under free uptake conditions with 10 uM of oligonucleotide in
differentiation media for four days. RNA was extracted with Agilent bead-based protocol
and reverse transcribed with random hexamers. TaqMan assays were targeted toward the total
DMD transcript or the exon-junction corresponding to the skipped transcript, each run in
multiplex with hSFSR9 internal control.
Numbers indicate amount of skipping DMD exon 51 (as a percentage of total DMD
mRNA, where 100 would represent 100% skipped).

| | | | | | | | | | |
|---|---|---|---|---|---|---|---|---|---|
| WV-3152 | 19 | 20 | 12 | 14 | WV-20094 | 55 | 50 | 57 | 55 |
| WV-15860 | 29 | 31 | 26 | 23 | WV-20093 | 35 | 34 | 35 | 38 |
| WV-20140 | 1 | 1 | 1 | 1 | WV-20092 | 25 | 26 | 25 | 25 |
| WV-20139 | 3 | 3 | 2 | 2 | WV-20091 | 28 | 27 | 30 | 32 |
| WV-20138 | | | 2 | 3 | WV-20090 | 21 | 19 | 22 | 22 |
| WV-20137 | 4 | 5 | | | WV-20089 | 8 | 7 | 8 | 9 |
| WV-20136 | | | | | WV-20088 | 22 | 21 | 26 | 25 |
| WV-20135 | 5 | 5 | 5 | 5 | WV-20087 | 28 | 28 | 33 | 32 |
| WV-20134 | 5 | 6 | 5 | 4 | WV-20086 | 25 | 25 | 27 | 26 |
| WV-20133 | 17 | 17 | 13 | 13 | WV-20085 | 33 | 31 | 30 | 31 |
| WV-20132 | 8 | 8 | 6 | 6 | WV-20084 | 21 | 22 | 21 | 21 |
| WV-20131 | 14 | 16 | 12 | 12 | WV-20083 | 21 | 21 | 19 | 17 |
| WV-20130 | 10 | 9 | 8 | 8 | WV-20082 | 42 | 37 | 32 | 30 |
| WV-20129 | 12 | 14 | 11 | 11 | WV-20081 | 41 | 41 | 30 | 30 |
| WV-20128 | 9 | 9 | 8 | 8 | WV-20080 | 49 | 44 | 26 | 25 |
| WV-20127 | | | 8 | 8 | WV-20079 | 42 | 38 | 53 | 51 |
| WV-20126 | 7 | 8 | 8 | 7 | WV-20078 | 27 | 28 | 36 | 35 |
| WV-20125 | 8 | 8 | 8 | 8 | WV-20077 | 10 | 10 | 10 | 10 |
| WV-20124 | 22 | 21 | 21 | 21 | WV-20076 | 45 | 45 | 45 | 41 |
| WV-20123 | 13 | 13 | 14 | 12 | WV-20075 | 40 | 31 | 37 | 42 |
| WV-20122 | 11 | 12 | 12 | 11 | WV-20074 | 55 | 57 | 53 | 56 |
| WV-20121 | 21 | 22 | 22 | 21 | WV-20073 | 51 | 55 | 51 | 50 |
| WV-20120 | 28 | 30 | 32 | 33 | WV-20072 | 41 | 36 | 37 | 36 |
| WV-20119 | 52 | 50 | | | WV-20071 | 42 | 40 | 44 | 46 |
| WV-20118 | 39 | 37 | 27 | 26 | WV-20070 | 18 | 18 | 25 | 25 |
| WV-20117 | 18 | 17 | 15 | 18 | WV-20069 | 11 | 11 | 10 | 9 |
| WV-20116 | 20 | 20 | 17 | 17 | WV-20068 | 20 | 17 | 20 | 18 |
| WV-20115 | 8 | 8 | 8 | 6 | WV-20067 | 12 | 9 | 11 | 11 |
| WV-20114 | 19 | 20 | 15 | 14 | WV-20066 | 12 | 11 | 13 | 12 |
| WV-20113 | 20 | 18 | 17 | 15 | WV-20065 | 16 | 15 | 16 | 14 |
| WV-20112 | 16 | 15 | 12 | 12 | WV-20064 | 37 | 35 | 37 | 36 |
| WV-20111 | 31 | 30 | 33 | 31 | WV-20063 | 19 | | 24 | 22 |
| WV-20110 | 14 | 14 | 14 | 12 | WV-20062 | 6 | 6 | 7 | 7 |
| WV-20109 | 20 | 21 | 25 | 24 | WV-20061 | 24 | 23 | 26 | 24 |
| WV-20108 | 27 | 25 | 22 | 22 | WV-20060 | 16 | 17 | 16 | 17 |
| WV-20107 | 20 | 19 | 16 | 14 | WV-20059 | 55 | 42 | 62 | 67 |
| WV-20106 | 44 | 42 | 34 | 37 | WV-20058 | 28 | 30 | 33 | 33 |
| WV-20105 | 23 | 22 | 18 | 18 | WV-20057 | 37 | 38 | 37 | 34 |
| WV-20104 | 41 | 40 | 33 | 28 | WV-20056 | 35 | 34 | 33 | 35 |
| WV-20103 | 48 | 52 | 53 | 53 | WV-20055 | | | 40 | 40 |
| WV-20102 | 54 | 52 | 55 | 59 | WV-20054 | 25 | 25 | 35 | 36 |
| WV-20101 | 38 | 39 | 38 | 43 | WV-20053 | 43 | 45 | 46 | 46 |
| WV-20100 | 52 | 51 | 48 | 50 | WV-20052 | 47 | 47 | 53 | 46 |
| WV-20099 | 53 | 51 | 47 | 48 | WV-20051 | 30 | 33 | 30 | 30 |
| WV-20098 | 46 | 44 | 45 | 46 | WV-20050 | 29 | 28 | 28 | 26 |
| WV-20097 | 47 | 46 | 51 | 48 | WV-20049 | 41 | 41 | 38 | 38 |
| WV-20096 | 45 | 41 | 42 | 43 | WV-20049 | 24 | 23 | 22 | 21 |
| WV-20095 | 43 | 41 | 50 | 47 | | | | | |

TABLE 4G
Activity of certain DMD oligonucleotides
Delta 48-50 cells were treated under free uptake conditions with 10 uM of oligonucleotide
in differentiation media for four days. RNA was extracted with Agilent bead-based
protocol and reverse transcribed with random hexamers. TaqMan assays were targeted
toward the total DMD transcript or the exon-junction corresponding to
the skipped transcript, each run in multiplex with hSFSR9 internal control.
Numbers indicate amount of skipping DMD exon 51 (as a percentage of total
DMD mRNA, where 100 would represent 100% skipped).

| | | | | | | | | | |
|---|---|---|---|---|---|---|---|---|---|
| WV-3152 | 19 | 20 | 12 | 14 | WV-20094 | 55 | 50 | 57 | 55 |
| WV-15860 | 29 | 31 | 26 | 23 | WV-20093 | 35 | 34 | 35 | 38 |
| WV-20140 | 1 | 1 | 1 | 1 | WV-20092 | 25 | 26 | 25 | 25 |
| WV-20139 | 3 | 3 | 2 | 2 | WV-20091 | 28 | 27 | 30 | 32 |
| WV-20138 | | | 2 | 3 | WV-20090 | 21 | 19 | 22 | 22 |
| WV-20137 | 4 | 5 | | | WV-20089 | 8 | 7 | 8 | 9 |
| WV-20136 | | | | | WV-20088 | 22 | 21 | 26 | 25 |
| WV-20135 | 5 | 5 | 5 | 5 | WV-20087 | 28 | 28 | 33 | 32 |
| WV-20134 | 5 | 6 | 5 | 4 | WV-20086 | 25 | 25 | 27 | 26 |
| WV-20133 | 17 | 17 | 13 | 13 | WV-20085 | 33 | 31 | 30 | 31 |
| WV-20132 | 8 | 8 | 6 | 6 | WV-20084 | 21 | 22 | 21 | 21 |
| WV-20131 | 14 | 16 | 12 | 12 | WV-20083 | 21 | 21 | 19 | 17 |
| WV-20130 | 10 | 9 | 8 | 8 | WV-20082 | 42 | 37 | 32 | 30 |
| WV-20129 | 12 | 14 | 11 | 11 | WV-20081 | 41 | 41 | 30 | 30 |
| WV-20128 | 9 | 9 | 8 | 8 | WV-20080 | 49 | 44 | 26 | 25 |
| WV-20127 | | | 8 | 8 | WV-20079 | 42 | 38 | 53 | 51 |
| WV-20126 | 7 | 8 | 8 | 7 | WV-20078 | 27 | 28 | 36 | 35 |
| WV-20125 | 8 | 8 | 8 | 8 | WV-20077 | 10 | 10 | 10 | 10 |
| WV-20124 | 22 | 21 | 21 | 21 | WV-20076 | 45 | 45 | 45 | 41 |
| WV-20123 | 13 | 13 | 14 | 12 | WV-20075 | 40 | 31 | 37 | 42 |
| WV-20122 | 11 | 12 | 12 | 11 | WV-20074 | 55 | 57 | 53 | 56 |
| WV-20121 | 21 | 22 | 22 | 21 | WV-20073 | 51 | 55 | 51 | 50 |
| WV-20120 | 28 | 30 | 32 | 33 | WV-20072 | 41 | 36 | 37 | 36 |
| WV-20119 | 52 | 50 | | | WV-20071 | 42 | 40 | 44 | 46 |
| WV-20118 | 39 | 37 | 27 | 26 | WV-20070 | 18 | 18 | 25 | 25 |
| WV-20117 | 18 | 17 | 15 | 18 | WV-20069 | 11 | 11 | 10 | 9 |
| WV-20116 | 20 | 20 | 17 | 17 | WV-20068 | 20 | 17 | 20 | 18 |
| WV-20115 | 8 | 8 | 8 | 6 | WV-20067 | 12 | 9 | 11 | 11 |
| WV-20114 | 19 | 20 | 15 | 14 | WV-20066 | 12 | 11 | 13 | 12 |
| WV-20113 | 20 | 18 | 17 | 15 | WV-20065 | 16 | 15 | 16 | 14 |
| WV-20112 | 16 | 15 | 12 | 12 | WV-20064 | 37 | 35 | 37 | 36 |
| WV-20111 | 31 | 30 | 33 | 31 | WV-20063 | 19 | | 24 | 22 |
| WV-20110 | 14 | 14 | 14 | 12 | WV-20062 | 6 | 6 | 7 | 7 |
| WV-20109 | 20 | 21 | 25 | 24 | WV-20061 | 24 | 23 | 26 | 24 |
| WV-20108 | 27 | 25 | 22 | 22 | WV-20060 | 16 | 17 | 16 | 17 |
| WV-20107 | 20 | 19 | 16 | 14 | WV-20059 | 55 | 42 | 62 | 67 |
| WV-20106 | 44 | 42 | 34 | 37 | WV-20058 | 28 | 30 | 33 | 33 |
| WV-20105 | 23 | 22 | 18 | 18 | WV-20057 | 37 | 38 | 37 | 34 |
| WV-20104 | 41 | 40 | 33 | 28 | WV-20056 | 35 | 34 | 33 | 35 |
| WV-20103 | 48 | 52 | 53 | 53 | WV-20055 | | | 40 | 40 |
| WV-20102 | 54 | 52 | 55 | 59 | WV-20054 | 25 | 25 | 35 | 36 |
| WV-20101 | 38 | 39 | 38 | 43 | WV-20053 | 43 | 45 | 46 | 46 |
| WV-20100 | 52 | 51 | 48 | 50 | WV-20052 | 47 | 47 | 53 | 46 |
| WV-20099 | 53 | 51 | 47 | 48 | WV-20051 | 30 | 33 | 30 | 30 |
| WV-20098 | 46 | 44 | 45 | 46 | WV-20050 | 29 | 28 | 28 | 26 |
| WV-20097 | 47 | 46 | 51 | 48 | WV-20049 | 41 | 41 | 38 | 38 |
| WV-20096 | 45 | 41 | 42 | 43 | WV-20049 | 24 | 23 | 22 | 21 |
| WV-20095 | 43 | 41 | 50 | 47 | | | | | |

TABLE 4H
Activity of certain DMD oligonucleotides
Delta 48-50 cells were treated under free uptake conditions with
10 uM of oligonucleotide in differentiation
media for four days. Numbers indicate amount of skipping
DMD exon 51 (as a percentage of total DMD mRNA,
where 100 would represent 100% skipped).
As in other experiments, multiple numbers
for an oligonucleotide indicate replicates.

| | | | | | |
|---|---|---|---|---|---|
| Mock | 0.2 | 0.2 | WV-20004 | 42.7 | 45.8 |
| WV-20119 | 47.7 | 45.9 | WV-20003 | 35.9 | 28.3 |
| WV-3152 | 25.2 | 15 | WV-20002 | 44.6 | 38.8 |
| WV-20064 | 44.5 | 30.4 | WV-20001 | 33 | 17.5 |
| WV-20048 | 38.3 | 36.8 | WV-20000 | 31.9 | 19.4 |
| WV-20047 | 37.2 | 35.1 | WV-19999 | 29.9 | 25.1 |
| WV-20045 | 45.4 | 38.7 | WV-19998 | 30.3 | 24.1 |
| WV-20044 | 30.4 | 29.6 | WV-19997 | 35 | 25.4 |
| WV-20043 | 38.3 | 32.1 | WV-19996 | 14.3 | 11.2 |
| WV-20042 | 42.3 | 34.6 | WV-19995 | 16.3 | 11.5 |
| WV-20041 | 37.9 | 25.2 | WV-19994 | 20.5 | 15 |
| WV-20040 | 31.4 | 24.9 | WV-19993 | 30.6 | 20.6 |
| WV-20039 | 39.3 | 30 | WV-19992 | 41.5 | 35.9 |
| WV-20038 | 27.8 | 15.4 | WV-19991 | 38.7 | 35.2 |
| WV-20037 | 34.6 | 24.2 | WV-19990 | 27.6 | 21.4 |
| WV-20036 | 22.9 | 21.5 | WV-19989 | 45 | 33 |
| WV-20035 | 27.8 | 16.2 | WV-19988 | 37.4 | 34.4 |
| WV-20034 | 25.1 | 13.3 | WV-19987 | 46 | 39.5 |
| WV-20033 | 36.8 | 26.3 | WV-19986 | 41.2 | 35.2 |
| WV-20032 | 34.1 | 22.6 | WV-19985 | 50.6 | 42.7 |
| WV-20031 | 37.4 | 29.3 | WV-19984 | 39 | 34.3 |
| WV-20030 | 32.4 | 31.9 | WV-19983 | 34.4 | 27.1 |
| WV-20029 | 45.1 | 32.6 | WV-19982 | 38 | 33.3 |
| WV-20028 | 39.3 | 41 | WV-19981 | 32.8 | 23.8 |
| WV-20027 | 45.5 | 38.8 | WV-19980 | 46.4 | 37 |
| WV-20026 | 41.1 | 28.3 | WV-19979 | 45.4 | 41.6 |
| WV-20025 | 43.6 | 32.6 | WV-19978 | 33.1 | 22.1 |
| WV-20024 | 32.1 | 20.4 | WV-19977 | 39.3 | 30.9 |
| WV-20023 | 29.5 | 19.1 | WV-19976 | 31 | 21.6 |
| WV-20022 | 43 | 39.3 | WV-19975 | 23.4 | 19.4 |
| WV-20021 | 49.2 | 33.1 | WV-19974 | 28 | 15.3 |
| WV-20020 | 43.3 | 42.3 | WV-19973 | 31.9 | 22 |
| WV-20019 | 26.5 | 16.4 | WV-19972 | 30.8 | 28.9 |
| WV-20018 | 43.2 | 37.4 | WV-19971 | 26.5 | 14.4 |
| WV-20017 | 49.6 | 33.2 | WV-19970 | 23.8 | 15.5 |
| WV-20016 | 48.9 | 45.1 | WV-19969 | 11.7 | 7.9 |
| WV-20015 | 45 | 40.7 | WV-19968 | 2.3 | 1.5 |
| WV-20014 | 44.1 | 39.3 | WV-19967 | 2.4 | 1.1 |
| WV-20013 | 64.3 | 40.8 | WV-19966 | 1.9 | 1 |
| WV-20012 | 48.5 | 46.3 | WV-19965 | 1.7 | 0.7 |
| WV-20011 | 54.5 | 49.9 | WV-19964 | 0.8 | 0.7 |
| WV-20010 | 46.4 | 34.6 | WV-19963 | 1.7 | 0.7 |
| WV-20009 | 51.2 | 44.7 | WV-19962 | 0.3 | 0.4 |
| WV-20008 | 45.6 | 43.2 | WV-19961 | 4.1 | 2.1 |
| WV-20007 | 43.5 | 39.3 | WV-19960 | 2.8 | 1.9 |
| WV-20006 | 43.9 | 38.8 | WV-19959 | 14 | 10.6 |
| WV-20005 | 41 | 28.8 | WV-19958 | 8.3 | 5.7 |
| | | | WV-19957 | 5.5 | 4.5 |

TABLE 4I
Activity of certain DMD oligonucleotides
Delta 48-50 cells were treated under free uptake conditions
with 1 uM of oligonucleotide in differentiation media for four days.
Numbers indicate amount of
skipping DMD exon 51 (as a percentage of total
DMD mRNA, where 100 would represent 100% skipped).

| | | | | | |
|---|---|---|---|---|---|
| Mock | 0.6 | 0.2 | WV-20003 | 4.9 | 12.6 |
| WV-3152 | 6 | 6.1 | WV-20002 | 26.8 | 27.4 |
| WV-20064 | 22.3 | 22 | WV-20001 | 13.3 | 15.4 |
| WV-20119 | 36.3 | 29.9 | WV-20000 | 10.1 | 17.5 |
| WV-20048 | 10.2 | 12.2 | WV-19999 | 3.9 | 4.8 |
| WV-20047 | 8.8 | 10 | WV-19998 | 5.1 | 5.6 |
| WV-20045 | 9.6 | 12.6 | WV-19997 | 11.8 | 13.4 |
| WV-20044 | 3.6 | 2.9 | WV-19996 | 3 | 2.7 |
| WV-20043 | 3.4 | 2.1 | WV-19995 | 0.9 | 1.3 |
| WV-20042 | 8.7 | 15.2 | WV-19994 | 4.1 | 4.3 |
| WV-20041 | 8.9 | 8.1 | WV-19993 | 9 | 10.7 |
| WV-20040 | 3.6 | 3.7 | WV-19992 | 25.2 | 22.4 |
| WV-20039 | 1.3 | 2.4 | WV-19991 | 7.8 | 14 |
| WV-20038 | 4 | 3 | WV-19990 | 5 | 4.5 |
| WV-20037 | 6.3 | 7.3 | WV-19989 | 16 | 22 |
| WV-20036 | 5.3 | 3.6 | WV-19988 | 9.3 | 5.9 |
| WV-20035 | 3.1 | 4.3 | WV-19987 | 4.5 | 10.3 |
| WV-20034 | 4.5 | 3.9 | WV-19986 | 6.2 | 5.2 |
| WV-20033 | 8.7 | 10.6 | WV-19985 | 13.9 | 14.8 |
| WV-20032 | 8.8 | 12.4 | WV-19984 | 7 | 6.8 |
| WV-20031 | 4 | 4.2 | WV-19983 | 1.2 | 3.5 |
| WV-20030 | 4.7 | 3.6 | WV-19982 | 2.2 | 4.1 |
| WV-20029 | 3 | 4.6 | WV-19981 | 2.1 | 4.4 |
| WV-20028 | 10 | 4.5 | WV-19980 | 4 | 2.7 |
| WV-20027 | 2.3 | 4.3 | WV-19979 | 4.4 | 9 |
| WV-20026 | 9.6 | 7.7 | WV-19978 | 4.1 | 4.5 |
| WV-20025 | 11.1 | 12.1 | WV-19977 | 5.4 | 8.1 |
| WV-20024 | 9.6 | 8.6 | WV-19976 | 4.5 | 3.1 |
| WV-20023 | 3.3 | 5.7 | WV-19975 | 2.4 | 2.7 |
| WV-20022 | 13.5 | 16.7 | WV-19974 | 5.5 | 6 |
| WV-20021 | 18.5 | 25.2 | WV-19973 | 10.5 | 9.5 |
| WV-20020 | 10.1 | 5.2 | WV-19972 | 10.1 | 8.6 |
| WV-20019 | 2.6 | 3.5 | WV-19971 | 3.6 | 4 |
| WV-20018 | 20.4 | 20.1 | WV-19970 | 7.3 | 11.2 |
| WV-20017 | 20.7 | 27.2 | WV-19969 | 3.4 | 5.5 |
| WV-20016 | 39.4 | 39.6 | WV-19968 | 1.4 | 1.2 |
| WV-20015 | 9.4 | 19.7 | WV-19967 | 0.8 | 1 |
| WV-20014 | 16.8 | 38.7 | WV-19966 | 0.6 | 1.1 |
| WV-20013 | 25.1 | 31.5 | WV-19965 | 0.4 | 0.7 |
| WV-20012 | 16.8 | 7.4 | WV-19964 | 0.4 | 0.3 |
| WV-20011 | 25.1 | 42.8 | WV-19963 | 0.5 | 0.6 |
| WV-20010 | 20.6 | 26.5 | WV-19962 | 0.6 | 1 |
| WV-20009 | 39.2 | 38.9 | WV-19961 | 1 | 1.4 |
| WV-20008 | 30.6 | 40.1 | WV-19960 | 0.8 | 1.1 |
| WV-20007 | 15.3 | 16.7 | WV-19959 | 2.1 | 4 |
| WV-20006 | 14 | 16.7 | WV-19958 | 2.8 | 4.5 |
| WV-20005 | 13.7 | 13.8 | WV-19957 | 1.4 | 1.9 |
| WV-20004 | 9 | 7.5 | | | |

TABLE 4J
Activity of certain DMD oligonucleotides
Delta 48-50 cells were treated under free uptake conditions with
10 uM of oligonucleotide in differentiation media for four days.
Numbers indicate amount of skipping DMD exon 51 (as a percentage
of total DMD mRNA, where 100 would represent 100% skipped).

| | | | | | |
|---|---|---|---|---|---|
| Mock | 0 | 0 | WV-19921 | 4 | 3 |
| WV-3152 | 56 | 51 | WV-19920 | 5 | 5 |
| WV-20064 | 73 | 64 | WV-19919 | 2 | 2 |
| WV-20102 | 78 | 81 | WV-19918 | 3 | 3 |
| WV-20119 | 79 | 80 | WV-19917 | 2 | 1 |
| WV-19953 | 18 | 17 | WV-19916 | 1 | 1 |
| WV-19952 | 22 | 21 | WV-19915 | 2 | 3 |
| WV-19951 | 32 | 27 | WV-19914 | 1 | 2 |
| WV-19950 | 52 | 46 | WV-19913 | 1 | 1 |
| WV-19949 | 31 | 35 | WV-19912 | 1 | 1 |
| WV-19948 | 18 | 22 | WV-19911 | 3 | 2 |
| WV-19947 | 14 | 11 | WV-19910 | 2 | 2 |
| WV-19946 | 18 | 15 | WV-19909 | 2 | 3 |
| WV-19945 | 10 | 10 | WV-19908 | 1 | 1 |
| WV-19944 | 37 | 34 | WV-19907 | 2 | 2 |
| WV-19943 | 15 | 14 | WV-19906 | 1 | 1 |
| WV-19942 | 43 | 45 | WV-19905 | 1 | 2 |
| WV-19941 | 38 | 37 | WV-19904 | 3 | 3 |
| WV-19940 | 37 | 40 | WV-19903 | 5 | 6 |
| WV-19939 | 36 | 36 | WV-19902 | 9 | 13 |
| WV-19938 | 20 | 20 | WV-19901 | 20 | 18 |
| WV-19937 | 4 | 4 | WV-19900 | 26 | 24 |
| WV-19936 | 10 | 10 | WV-19899 | 20 | 23 |
| WV-19935 | 11 | 10 | WV-19898 | 17 | 18 |
| WV-19934 | 14 | 12 | WV-19897 | 3 | 4 |
| WV-19933 | 9 | 10 | WV-19896 | 2 | 2 |
| WV-19932 | 15 | 18 | WV-19895 | 0 | 0 |
| WV-19931 | 12 | 13 | WV-19894 | 0 | 0 |
| WV-19930 | 13 | 13 | WV-19893 | 0 | 0 |
| WV-19929 | 26 | 24 | WV-19892 | 0 | 0 |
| WV-19928 | 17 | 17 | WV-19891 | 0 | 0 |
| WV-19927 | 7 | 6 | WV-19890 | 0 | 0 |
| WV-19926 | 8 | 9 | WV-19889 | 0 | 0 |
| WV-19925 | 3 | 3 | WV-19888 | 0 | 0 |
| WV-19924 | 3 | 3 | WV-19887 | 0 | 0 |
| WV-19923 | 3 | 3 | WV-19886 | 0 | 0 |
| WV-19922 | 3 | 3 | | | |

TABLE 4K
Activity of certain DMD oligonucleotides
Delta 48-50 cells were treated under free uptake conditions with 3,
1 or 0.3 uM of oligonucleotide in differentiation media for three days.
Numbers indicate amount of skipping DMD exon 51 (as a percentage of
total DMD mRNA, where 100 would represent 100% skipped).

| | 3 uM | | l uM | | 0.3 uM | |
|---|---|---|---|---|---|---|
| Water | 0.1 | 0.1 | 0.3 | 0.1 | 0.2 | 0.4 |
| WV-3152 | 12.4 | 12 | 4.3 | 3.8 | 1.6 | 1.5 |
| WV-20064 | 23.6 | 22.3 | 6.9 | 4.7 | 1.7 | 0.4 |
| WV-20120 | 17.9 | 21.6 | 3.3 | 4.4 | 0.9 | 0.9 |
| WV-20119 | 30.3 | 38.3 | 7.4 | 7 | 1.6 | 1.5 |
| WV-20118 | 26.4 | 29.4 | 6.5 | 5 | 1.5 | 1 |
| WV-20103 | 26.2 | 27.6 | 8.9 | 9.6 | 1.7 | 1.9 |
| WV-20102 | 25.7 | 29 | 11.6 | 12.8 | 1.8 | 2.7 |
| WV-20101 | 27.1 | 30.2 | 8.8 | 10.3 | 1.8 | 1.7 |
| WV-20100 | 33.9 | 35.7 | 9.9 | 11.6 | 2.1 | 2.2 |
| WV-20099 | 37.4 | 38.3 | 12.1 | 12.4 | 2.7 | 2.1 |
| WV-20098 | 35.5 | 34.1 | 11.1 | 12.9 | 2.2 | 2.5 |
| WV-20097 | 24.1 | 28.6 | 10.6 | 8.3 | 2.4 | 2.2 |
| WV-20096 | 29.8 | 29.9 | 8.9 | 8.9 | 1.2 | 2.2 |
| WV-20095 | 42.2 | 37.7 | 8.7 | 9.1 | 1.8 | 2 |
| WV-20094 | 36.7 | 43.9 | 10.9 | 14.9 | 2.2 | 2.1 |
| WV-20076 | 28.4 | 32 | 8.8 | 10.1 | 1.9 | 2 |
| WV-20075 | 37.5 | 36.7 | 9.1 | 11.1 | 1.7 | 2 |
| WV-20074 | 35.8 | 43.5 | 10.5 | 11.4 | 2 | 2 |
| WV-20073 | 29.7 | 35.4 | 9.1 | 8.1 | 1.3 | 1.5 |
| WV-20072 | 30.2 | 34.4 | 4.9 | 5.5 | 0.7 | 0.7 |
| WV-20071 | 33.7 | 37.5 | 10.8 | 8.8 | 1.8 | 1.6 |
| WV-20059 | 34.8 | 42.8 | 16.4 | 15.7 | 2.6 | 2.5 |
| WV-20058 | 22.9 | 26 | 5.3 | 6 | 1.1 | 0.8 |
| WV-20057 | 20.4 | 30.7 | 5.8 | 7.9 | 1.7 | 1.2 |
| WV-20054 | 17.1 | 27 | 5.1 | 5.1 | 1.1 | 1.5 |
| WV-20053 | 48.8 | 36.2 | 6.6 | 11.8 | 2.1 | 1.9 |
| WV-20052 | 46.5 | 52.9 | 13.9 | 16.5 | 1.5 | 3.6 |
| WV-20021 | 27.5 | 26.8 | 5.6 | 5.7 | 0 | 0 |
| WV-20018 | 32.2 | 24.2 | 5.1 | 4.9 | 1.2 | 1.5 |
| WV-20017 | 34.5 | 36.6 | 4.1 | 5 | 1.2 | 1 |
| WV-20016 | 28.8 | 32.4 | 5.7 | 6.9 | 1.6 | 2.1 |
| WV-20015 | 29 | 54 | 10 | 12 | 2 | 2 |
| WV-20014 | 21 | 39 | 8 | 5 | 1 | 2 |
| WV-20013 | 13 | 14 | 9 | 6 | 1 | 1 |
| WV-20012 | 42 | 44 | 12 | 12 | 2 | 2 |
| WV-20011 | 39 | 64 | 20 | 17 | 3 | 5 |
| WV-20010 | 28 | 33 | 7 | 11 | 2 | 2 |
| WV-20009 | 35 | 36 | 12 | 11 | 2 | 2 |
| WV-20008 | 34 | 34 | 13 | 10 | 1 | 2 |
| WV-20007 | 52 | 31 | 6 | 6 | 0 | 1 |
| WV-20002 | 27 | 49 | 8 | 7 | 1 | 1 |
| WV-19992 | 28 | 35 | 7 | 5 | 1 | 1 |
| WV-19989 | 20 | 22 | 6 | 5 | 2 | 1 |
| WV-19950 | 22 | 37 | 8 | 6 | 3 | 2 |

TABLE 5A
Activity of certain DMD oligonucleotides.
Delta 48-50 cells were treated under free uptake conditions with
5 or 1 uM of oligonucleotide in differentiation media for four days.
Numbers indicate amount of skipping DMD exon 51 (as a percentage of
total DMD mRNA, where 100 would represent 100% skipped).

| | 5 uM | | 1 uM | |
|---|---|---|---|---|
| WV-20052 | 4.7 | 3.1 | 0.9 | 1.4 |
| WV-31538 | 1.4 | 0.7 | 0.3 | 0.1 |
| WV-31550 | 1.2 | 1.1 | 0.3 | 0.2 |
| WV-31562 | 3.5 | 2.4 | 0.6 | 0.5 |
| WV-31574 | 3.4 | 1.4 | 0.7 | 1.1 |
| WV-31200 | 2.5 | 2 | 0.1 | 0.2 |
| WV-20074 | 6.3 | 6.8 | 1.3 | 1.2 |
| WV-31541 | 2.4 | 1.6 | 0.5 | 0.5 |
| WV-31553 | 1.9 | 2.6 | 0.1 | 0.4 |
| WV-31565 | 6 | 4.6 | 0.6 | 0.8 |
| WV-31577 | 1.7 | 0.5 | 0.5 | 0.4 |
| WV-31211 | 2.1 | | | 0.2 |
| WV-20075 | 2.9 | 3 | 0.6 | 1.8 |
| WV-31542 | 1.6 | 2.1 | 0.8 | 0.4 |
| WV-31554 | 2 | 3 | 1.1 | 1 |
| WV-31566 | 2.7 | 3.5 | 1.5 | 1.5 |
| WV-31578 | 3.1 | 4 | 2 | 2.3 |
| WV-31212 | 1 | 1.7 | 0 | 0 |
| WV-20094 | 7.6 | 5.5 | 1.9 | 1.7 |
| WV-31544 | 1.6 | 1.4 | 0.4 | 0.2 |
| WV-31556 | 1.2 | 1.6 | 0.9 | 0.9 |
| WV-31568 | 2.9 | 3.6 | 0.5 | 0.4 |
| WV-31580 | 1.5 | 4.5 | 0.4 | 0.3 |
| WV-31214 | 2.1 | | 0.1 | 0.1 |

TABLE 5B
Activity of certain DMD oligonucleotides.
Delta 48-50 cells were treated under free uptake conditions with
5 uM of oligonucleotide in differentiation media for four days.
Numbers indicate amount of skipping DMD exon 51 (as a percentage
of total DMD mRNA, where 100 would represent 100% skipped).

| | 5 uM | | | 5 uM | |
|---|---|---|---|---|---|
| H₂O | 0 | 0 | WV-31567 | 5.5 | 4.6 |
| WV-3152 | 2.8 | 2.9 | WV-31579 | 3.7 | 4 |
| WV-20011 | 9.7 | 11.5 | WV-31588 | 3.8 | 3.8 |
| WV-31537 | 6.3 | 5.7 | WV-20097 | 10.6 | 10.8 |
| WV-31549 | 3.6 | 3.4 | WV-31545 | 2.8 | 2.8 |
| WV-31561 | 6.9 | 7.5 | WV-31557 | 1.9 | 2.1 |
| WV-31573 | 7.9 | 8.1 | WV-31569 | 7.9 | 8.1 |
| WV-31585 | 2.8 | 2.7 | WV-31581 | 4.6 | 4.4 |
| WV-20059 | 9.2 | 9 | | | |
| WV-31539 | 3 | 3.1 | WV-20098 | 7.4 | 7.9 |
| WV-31551 | 2.3 | 2.2 | WV-31546 | 4.7 | 4.5 |
| WV-31563 | 5.6 | 5.5 | WV-31558 | 5.2 | 5.6 |
| WV-31575 | 3.6 | 3.7 | WV-31570 | 6.4 | 7.1 |
| WV-31586 | 2.5 | 2.4 | WV-31582 | 8.5 | 8.4 |
| WV-20073 | 10.1 | 10.6 | | | |
| WV-31540 | 3.6 | 3.8 | WV-20101 | 6.2 | 6.5 |
| WV-31552 | 4.2 | 4.1 | WV-31547 | 3 | 3.5 |
| WV-31564 | 7.7 | 7.6 | WV-31559 | 2 | 2.2 |
| WV-31576 | 8 | 7.5 | WV-31571 | 4.5 | 5.1 |
| WV-31587 | 2.9 | 3 | WV-31583 | 5.5 | 5.6 |
| WV-20076 | 8.6 | 8.4 | | | |
| WV-31543 | 2.7 | 2.6 | WV-20119 | 7.3 | 7.1 |
| WV-31555 | 1.8 | 1.7 | WV-31548 | 1.4 | 1.4 |
| WV-31572 | 3.9 | 3.9 | WV-31560 | 1.4 | 1.6 |
| WV-31584 | 4.9 | 5 | | | |

TABLE 5C
Activity of certain DMD oligonucleotides.
Delta 48-50 cells were treated under free uptake conditions with 5
or 1 uM of oligonucleotide in differentiation media for four days.
Numbers indicate amount of skipping DMD exon 51 (as a percentage
of total DMD mRNA, where 100 would represent 100% skipped).

| | 5 uM | | 1 uM | |
|---|---|---|---|---|
| water | 0 | 0 | 0 | 0 |
| WV-3152 | 13.2 | 9.3 | 3.3 | 3.4 |
| WV-31562 | 26.2 | 17 | 5.5 | 4.1 |
| WV-31574 | | 16.3 | 5.2 | 5.2 |
| WV-31565 | 47.5 | 23.7 | 11.1 | 10.6 |
| WV-31566 | 26 | 16.7 | 4.9 | 4.6 |
| WV-31578 | 34 | 23.7 | 7.2 | 8.2 |
| WV-31568 | 40.2 | 34.7 | 9.1 | 8.6 |
| WV-31580 | 40.6 | 27.6 | 8.8 | 9.3 |
| WV-31561 | 36.3 | 22.4 | 8.8 | 7.5 |
| WV-31573 | 36.5 | 28.2 | 9.6 | 7.9 |
| WV-31563 | 26.2 | 24.7 | 5.7 | 4.5 |
| WV-31564 | 38.2 | 33.6 | 9.5 | 6.8 |
| WV-31576 | 45 | 38.8 | 11.5 | 8.2 |
| WV-31567 | 26.4 | 19.8 | 6 | 4.1 |
| WV-31579 | 20.6 | 23.4 | 4.2 | 4.1 |
| WV-31569 | 38.8 | 26.5 | 6.5 | 5.1 |
| WV-31581 | 19.6 | 16.6 | 4 | 2.6 |
| WV-31570 | 26.6 | 26.2 | 6.8 | 6.4 |
| WV-31582 | 43.1 | 27.3 | 13 | 10.8 |
| WV-31571 | 18.6 | 13.9 | 3.1 | 3.7 |
| WV-31583 | 24.8 | 18.1 | 4.6 | 4.9 |
| WV-31572 | 18 | 14 | 2.8 | 3.4 |
| WV-31584 | 20.5 | 17.9 | 4.3 | 3.9 |

TABLE 5D
Activity of certain DMD oligonucleotides.
Delta 48-50 cells were treated under free uptake conditions with 5 or
1 uM of oligonucleotide in differentiation media for four days.
Numbers indicate amount of skipping DMD exon 51 (as a percentage
of total DMD mRNA, where 100 would represent 100% skipped).

| | 5 uM | | 1 uM | |
|---|---|---|---|---|
| Mock | 0 | 0 | 0 | 0 |
| WV-3152 | 1.9 | 1 | 0.7 | 0 |
| WV-20011 | 5.8 | 6 | 3.3 | 3 |
| WV-20059 | 7.4 | 5.1 | 2.7 | 1.1 |
| WV-20052 | 5.3 | 3 | 2.2 | 0.9 |
| WV-30285 | 5.9 | 5.4 | 1.9 | 0.6 |
| WV-20053 | 6.7 | 5.1 | 1.7 | 1.5 |
| WV-20071 | 5.3 | 3.5 | 1.5 | 0.7 |
| WV-20072 | 3.6 | 4.3 | 0.6 | 0.2 |
| WV-20073 | 6.6 | 2.4 | 0.6 | 0.9 |
| WV-20074 | 6.2 | 1.1 | 1.7 | 1.9 |
| WV-20075 | 3.2 | 2.4 | 1.2 | 1.2 |
| WV-20076 | 4.1 | 4.5 | 1.9 | 2 |
| WV-20096 | 4.4 | 8.1 | 2.6 | 2 |
| WV-30233 | 7.7 | 4.6 | 3 | 1.7 |
| WV-20097 | 6 | 7 | 3 | 2.6 |
| WV-20098 | 5.1 | 4.3 | 1.3 | 1.4 |
| WV-30234 | 5.6 | 7.8 | 0.7 | 1.9 |
| WV-20099 | 8.2 | 6.8 | 2.5 | 1.8 |
| WV-20009 | 9.2 | 8.5 | 2.3 | 0.9 |
| WV-30235 | 1.7 | 0.5 | 0.8 | 0.1 |
| WV-30236 | 4.2 | 0.6 | 0.2 | 0.1 |

TABLE 5E
Activity of certain DMD oligonucleotides.
Delta 48-50 cells were treated under free
uptake conditions with 5 uM of oligonucleotide
in differentiation media for four days.
Numbers indicate amount of skipping
DMD exon 51 (as a percentage of total
DMD mRNA, where 100 would represent 100% skipped).

| | 5 uM | | | 5 uM | |
|---|---|---|---|---|---|
| water | 0.1 | 0.0 | WV-31197 | 0.0 | 2.0 |
| WV-3152 | 2.0 | 2.1 | WV-31198 | 1.4 | 1.6 |
| WV-31565 | 5.0 | 5.5 | WV-31199 | 1.1 | 1.1 |
| WV-31578 | 4.2 | 4.8 | WV-31200 | 1.6 | 1.6 |
| WV-31567 | 3.0 | 4.1 | WV-31201 | 1.3 | 1.4 |
| WV-31569 | 7.4 | 6.7 | WV-31202 | 1.3 | 1.1 |
| WV-31582 | 8.5 | 8.2 | WV-31203 | 1.1 | 1.0 |
| WV-32693 | 8.5 | 7.3 | WV-31204 | 0.8 | 0.9 |
| WV-32694 | 11.2 | 11.6 | WV-31205 | 1.1 | 1.3 |
| WV-31570 | 6.6 | 6.0 | WV-31206 | 0.8 | 0.8 |
| WV-31561 | 4.0 | 3.9 | WV-31207 | 2.2 | 1.9 |
| WV-31573 | 5.5 | 5.6 | WV-31208 | 1.3 | 1.3 |
| WV-31179 | 1.6 | 1.8 | WV-31209 | 1.6 | 1.7 |
| WV-31180 | 1.0 | 1.1 | WV-31210 | 3.4 | 3.6 |
| WV-31181 | 0.9 | 0.8 | WV-31211 | 3.4 | 3.4 |
| WV-31182 | 1.3 | 1.6 | WV-31212 | 1.7 | 1.9 |
| WV-31183 | 1.0 | 0.9 | WV-31213 | 1.7 | 1.7 |
| WV-31184 | 1.0 | 0.6 | WV-31214 | 3.1 | 2.9 |
| WV-31185 | 1.3 | 1.3 | WV-31215 | 1.5 | 2.0 |
| WV-31186 | 1.4 | 1.5 | WV-31216 | 1.9 | 2.2 |
| WV-31187 | 1.0 | 1.1 | WV-31217 | 2.6 | 3.7 |
| WV-31188 | 1.8 | 2.0 | WV-31218 | 1.4 | 1.8 |
| WV-31189 | 3.2 | 3.1 | WV-31219 | 2.5 | 2.6 |
| WV-31190 | 2.7 | 2.4 | WV-31220 | 2.3 | 1.9 |
| WV-31191 | 3.2 | 3.0 | WV-31221 | 1.8 | 1.3 |
| WV-31192 | 3.5 | 4.0 | WV-31222 | 1.9 | 1.5 |
| WV-31193 | 0.0 | 2.1 | WV-31223 | 1.3 | 1.1 |
| WV-31194 | 0.0 | 1.9 | WV-31224 | 1.1 | 1.0 |
| WV-31195 | 0.0 | 2.6 | WV-31225 | 2.1 | 1.7 |
| WV-31196 | 0.0 | 2.1 | WV-31226 | 0.9 | 0.9 |

TABLE 5F
Activity of certain DMD oligonucleotides.
Delta 48-50 cells were treated under free uptake conditions with
5 uM of oligonucleotide in differentiation media for four days.
Numbers indicate amount of skipping DMD exon 51 (as a percentage of
total DMD mRNA, where 100 would represent 100% skipped).

| | 5 uM | | | 5 uM | |
|---|---|---|---|---|---|
| water | 0.0 | 0.0 | WV-31197 | 0.0 | 1.6 |
| WV-3152 | 2.7 | 1.3 | WV-31198 | 2.2 | 2.4 |
| WV-31565 | 8.4 | 11.0 | WV-31199 | 1.5 | 1.6 |
| WV-31578 | 7.2 | 8.7 | WV-31200 | 1.9 | 2.4 |
| WV-31567 | 2.7 | 7.3 | WV-31201 | 1.8 | 2.4 |
| WV-31569 | 14.3 | 14.7 | WV-31202 | 1.7 | 1.7 |
| WV-31582 | 14.7 | 16.9 | WV-31203 | 0.8 | 1.4 |
| WV-32693 | 15.6 | 15.0 | WV-31204 | 1.7 | 1.2 |
| WV-32694 | 24.1 | 23.5 | WV-31205 | 1.0 | 2.1 |
| WV-31570 | 6.9 | 10.6 | WV-31206 | 1.5 | 1.2 |
| WV-31561 | 6.8 | 4.9 | WV-31207 | 0.0 | 1.7 |
| WV-31573 | 8.8 | 8.6 | WV-31208 | 0.0 | 1.4 |
| WV-31179 | 2.3 | 2.8 | WV-31209 | 0.0 | 2.5 |
| WV-31180 | 1.7 | 1.0 | WV-31210 | 6.4 | 5.8 |
| WV-31181 | 1.2 | 1.1 | WV-31211 | 6.2 | 4.5 |
| WV-31182 | 2.0 | 2.2 | WV-31212 | 2.8 | 3.0 |
| WV-31183 | 1.6 | 1.5 | WV-31213 | 2.8 | 2.7 |
| WV-31184 | 1.2 | 1.3 | WV-31214 | 4.7 | 4.4 |
| WV-31185 | 1.8 | 1.9 | WV-31215 | 2.3 | 2.2 |
| WV-31186 | 1.9 | 2.3 | WV-31216 | 2.5 | 2.4 |
| WV-31187 | 1.6 | 1.8 | WV-31217 | 4.9 | 1.7 |
| WV-31188 | 2.6 | 2.9 | WV-31218 | 2.7 | 2.6 |
| WV-31189 | 4.9 | 4.9 | WV-31219 | 4.7 | 4.0 |
| WV-31190 | 3.8 | 3.8 | WV-31220 | 3.5 | 1.8 |
| WV-31191 | 5.2 | 4.5 | WV-31221 | 2.8 | 2.3 |
| WV-31192 | 5.9 | 6.6 | WV-31222 | 2.8 | 2.3 |
| WV-31193 | 0.0 | 4.1 | WV-31223 | 1.8 | 1.7 |
| WV-31194 | 0.0 | 2.7 | WV-31224 | 1.5 | 1.6 |
| WV-31195 | 0.0 | 1.3 | WV-31225 | 3.5 | 1.6 |
| WV-31196 | 4.0 | 6.2 | WV-31226 | 1.8 | 1.5 |

Additional information related to DMD oligonucleotides, the activity thereof, the synthesis and use thereof, and other aspects thereof, is available in International patent app1ications WO 2019/200185 and WO 2019/217784, the DMD oligonucleotides of which are herein incorporated by reference.

### Example Methods for Preparing Oligonucleotides and Compositions

Among other things, the present disclosure provides technologies (methods, reagents, conditions, purification processes, etc.) for preparing oligonucleotides and oligonucleotide compositions, including chirally controlled oligonucleotides and chirally controlled oligonucleotide nucleotides. Various technologies (methods, reagents, conditions, purification processes, etc.), as described herein, can be utilized to prepare provided oligonucleotides and compositions thereof in accordance with the present disclosure, including but not limited to those described in U.S. Pat. Nos. 9,695,211, 9,605,019, 9,598,458, US 2013/0178612, US 20150211006. US 20170037399, WO 2017/015555, WO 2017/062862, WO 2017/160741, WO 2017/192664, WO 2017/192679, WO 2017/210647, WO 2018/223056, WO 2018/237194, WO 2019/055951, WO 2019/200185, and/or WO 2019/217784, the preparation technologies of each of which are incorporated herein by reference.

In some embodiments, the present disclosure provides chirally controlled oligonucleotides, e.g., chirally controlled DMD oligonucleotides. In some embodiments, a provided chirally controlled DMD oligonucleotide is over 50% pure. In some embodiments, a provided chirally controlled DMD oligonucleotide is over about 55% pure. In some embodiments, a provided chirally controlled DMD oligonucleotide is over about 60% pure. In some embodiments, a provided chirally controlled DMD oligonucleotide is over about 65% pure. In some embodiments, a provided chirally controlled DMD oligonucleotide is over about 70% pure. In some embodiments, a provided chirally controlled DMD oligonucleotide is over about 75% pure. In some embodiments, a provided chirally controlled DMD oligonucleotide is over about 80% pure. In some embodiments, a provided chirally controlled DMD oligonucleotide is over about 85% pure. In some embodiments, a provided chirally controlled DMD oligonucleotide is over about 90% pure. In some embodiments, a provided chirally controlled DMD oligonucleotide is over about 91% pure. In some embodiments, a provided chirally controlled DMD oligonucleotide is over about 92% pure. In some embodiments, a provided chirally controlled DMD oligonucleotide is over about 93% pure. In some embodiments, a provided chirally controlled DMD oligonucleotide is over about 94% pure. In some embodiments, a provided chirally controlled DMD oligonucleotide is over about 95% pure. In some embodiments, a provided chirally controlled DMD oligonucleotide is over about 96% pure. In some embodiments, a provided chirally controlled DMD oligonucleotide is over about 97% pure. In some embodiments, a provided chirally controlled DMD oligonucleotide is over about 98% pure. In some embodiments, a provided chirally controlled DMD oligonucleotide is over about 99% pure. In some embodiments, a provided chirally controlled DMD oligonucleotide is over about 99.5% pure. In some embodiments, a provided chirally controlled DMD oligonucleotide is over about 99.6% pure. In some embodiments, a provided chirally controlled DMD oligonucleotide is over about 99.7% pure. In some embodiments, a provided chirally controlled DMD oligonucleotide is over about 99.8% pure. In some embodiments, a provided chirally controlled DMD oligonucleotide is over about 99.9% pure. In some embodiments, a provided chirally controlled DMD oligonucleotide is over at least about 99/o pure.

In some embodiments, a chirally controlled oligonucleotide composition, e.g., a chirally controlled DMD oligonucleotide composition, is a composition designed to comprise a single oligonucleotide type. In certain embodiments, such compositions are about 50% diastereomerically pure. In some embodiments, such compositions are about 50% diastereomerically pure. In some embodiments, such compositions are about 50% diastereomerically pure. In some embodiments, such compositions are about 55% diastereomerically pure. In some embodiments, such compositions are about 60% diastereomerically pure. In some embodiments, such compositions are about 65% diastereomerically pure. In some embodiments, such compositions are about 70% diastereomerically pure. In some embodiments, such compositions are about 75% diastereomerically pure. In some embodiments, such compositions are about 80% diastereomerically pure. In some embodiments, such compositions are about 85% diastereomerically pure. In some embodiments, such compositions are about 90% diastereomerically pure. In some embodiments, such compositions are about 91% diastereomerically pure. In some embodiments, such compositions are about 92% diastereomerically pure. In some embodiments, such compositions are about 93% diastereomerically pure. In some embodiments, such compositions are about 94% diastereomerically pure. In some embodiments, such compositions are about 95% diastereomerically pure. In some embodiments, such compositions are about 96% diastereomerically pure. In some embodiments, such compositions are about 97% diastereomerically pure. In some embodiments, such compositions are about 98% diastereomerically pure. In some embodiments, such compositions are about 99% diastereomerically pure. In some embodiments, such compositions are about 99.5% diastereomerically pure. In some embodiments, such compositions are about 99.6% diastereomerically pure. In some embodiments, such compositions are about 99.7% diastereomerically pure. In some embodiments, such compositions are about 99.8% diastereomerically pure. In some embodiments, such compositions are about 99.9% diastereomerically pure. In some embodiments, such compositions are at least about 99% diastereomerically pure.

Among other things, the present disclosure recognizes the challenge of stereoselective (rather than stereorandom or racemic) preparation of oligonucleotides, e.g., DMD oligonucleotides. Among other things, the present disclosure provides methods and reagents for stereoselective preparation of oligonucleotides comprising multiple (e.g., more than 5, 6, 7, 8, 9, or 10) internucleotidic linkages, and particularly for DMD oligonucleotides comprising multiple (e.g., more than 5, 6, 7, 8, 9, or 10) chiral internucleotidic linkages. In some embodiments, in a stereorandom or racemic preparation of oligonucleotides such as DMD oligonucleotides, at least one chiral internucleotidic linkage is formed with less than 90:10, 95:5, 96:4, 97:3, or 98:2 diastereoselectivity. In some embodiments, for a stereoselective or chirally controlled preparation of oligonucleotides such as DMD oligonucleotides, each chiral internucleotidic linkage is formed with greater than 90:10, 95:5, 96:4, 97:3, or 98:2 diastereoselectivity. In some embodiments, for a stereoselective or chirally controlled preparation of DMD oligonucleotides, each chiral internucleotidic linkage is formed with greater than 95:5 diastereoselectivity. In some embodiments, for a stereoselective or chirally controlled preparation of DMD oligonucleotides, each chiral internucleotidic linkage is formed with greater than 96:4 diastereoselectivity. In some embodiments, for a stereoselective or chirally controlled preparation of DMD oligonucleotides, each chiral internucleotidic linkage is formed with greater than 97:3 diastereoselectivity. In some embodiments, for a stereoselective or chirally controlled preparation of DMD oligonucleotides, each chiral internucleotidic linkage is formed with greater than 98:2 diastereoselectivity. In some embodiments, for a stereoselective or chirally controlled preparation of DMD oligonucleotides, each chiral internucleotidic linkage is formed with greater than 99:1 diastereoselectivity. In some embodiments, diastereoselectivity of a chiral internucleotidic linkage in an oligonucleotide, e.g., a DMD oligonucleotide may be measured through a model reaction, e.g. formation of a dimer under essentially the same or comparable conditions wherein the dimer has the same internucleotidic linkage as the chiral internucleotidic linkage, the 5′-nucleoside of the dimer is the same as the nucleoside to the 5′-end of the chiral internucleotidic linkage, and the 3′-nucleoside of the dimer is the same as the nucleoside to the 3′-end of the chiral internucleotidic linkage.

In some embodiments, a chirally controlled DMD oligonucleotide composition is a composition designed to comprise multiple DMD oligonucleotide types. In some embodiments, methods of the present disclosure allow for the generation of a library of chirally controlled DMD oligonucleotides such that a pre-selected amount of any one or more chirally controlled DMD oligonucleotide types can be mixed with any one or more other chirally controlled DMD oligonucleotide types to create a chirally controlled DMD oligonucleotide composition. In some embodiments, the pre-selected amount of a DMD oligonucleotide type is a composition having any one of the above-described diastereomeric purities.

In some embodiments, the present disclosure provides methods for making a chirally controlled oligonucleotide (e.g., a DMD oligonucleotide) comprising steps of:
(1) coupling;
(2) capping;
(3) optionally modifying;
(4) deblocking; and
(5) repeating steps (1)-(4) until a desired length is achieved.

In some embodiments, the present disclosure provides a method, e.g., for preparing a DMD oligonucleotide, comprising one or more cycles, each of which independently comprises:
(1) a coupling step:
(2) optionally a pre-modification capping step;
(3) a modification step;
(4) optionally a post-modification capping step; and
(5) optionally a de-blocking step.

In some embodiments, a cycle comprises one or more pre-modification capping steps. In some embodiments, a cycle comprises one or more post-modification capping steps. In some embodiments, a cycle comprises one or more pre- and post-modification capping steps. In some embodiments, a cycle comprises one or more de-blocking steps. In some embodiments, a cycle comprises a coupling step, a pre-modification capping step, a modification step, a post-modification capping step, and a de-blocking step. In some embodiments, a cycle comprises a coupling step, a pre-modification capping step, a modification step, and a de-blocking step. In some embodiments, a cycle comprises a coupling step, a modification step, a post-modification capping step and a de-blocking step. In some embodiments, comprise a coupling step, a pre-modification capping step, a modification step, a post-modification capping step, and a de-blocking step. In some embodiments, one or more cycles comprise a coupling step, a pre-modification capping step, a modification step, and a de-blocking step. In some embodiments, one or more cycles comprise a coupling step, a modification step, a post-modification capping step and a de-blocking step.

When describing the provided methods, the word “cycle” has its ordinary meaning as understood by a person of ordinary skill in the art. In some embodiments, one round of steps (1)-(4) is referred to as a cycle. In some embodiments, some cycles comprise modifying. In some embodiments, some cycles do not comprise modifying. In some embodiments, some cycles comprise and some cycles do not comprise modifying. In some embodiments, each cycle independently comprises a modifying step. In some embodiments, each cycle does not comprise a cycling step.

In some embodiments, to form a chirally controlled internucleotidic linkage, a chirally pure phosphoramidite comprising a chiral auxiliary is utilized to stereoselectively form the chirally controlled internucleotidic linkage. Various phosphoramidite and chiral auxiliaries, e.g., those described in U.S. Pat. No. 9,695,211. U.S. Pat. Nos. 9,605,019, 9,598,458, US 2013/0178612, US 20150211006, US 20170037399, WO 2017/015555, WO 2017/062862, WO 2017/160741, WO 2017/192664, WO 2017/192679, WO 2017/210647, WO 2018/223056, WO 2018/237194, WO 2019/055951, WO 2019/200185, and/or WO 2019/217784, the phosphoramidite and chiral auxiliaries of each of which are incorporated herein by reference, may be utilized in accordance with the present disclosure.

In some embodiments, such an internucleotidic linkage is a neutral internucleotidic linkage. In some embodiments, such an internucleotidic linkage is a chirally controlled internucleotidic linkage. In some embodiments, such an internucleotidic linkage comprises a chiral auxiliary moiety. In some embodiments, such an internucleotidic linkage comprises no chiral auxiliary moiety. In some embodiments, a chiral auxiliary moiety falls off during modification.

Provided technologies provide various advantages. Among other things, as demonstrated herein, provided technologies can greatly improve oligonucleotide synthesis crude purity and yield, particularly for modified and/or chirally pure oligonucleotides such as DMD oligonucleotides that provide a number of properties and activities that are critical for therapeutic purposes. With the capability to provide unexpectedly high crude purity and yield for therapeutically important DMD oligonucleotides, provided technologies can significantly reduce manufacturing costs (through, e.g., simplified purification, greatly improved overall yields, etc.). In some embodiments, provided technologies can be readily scaled up to produce DMD oligonucleotides in sufficient quantities and qualities for clinical purposes. In some embodiments, provided technologies comprising chiral auxiliaries that comprise electron-withdrawing groups in G² (e.g., PSM chiral auxiliaries) are particularly useful for preparing chirally controlled internucleotidic linkages comprising P—N bonds (e.g., non-negatively charged internucleotidic linkages such as n001) and can significantly simplify manufacture operations, reduce cost, and/or facilitate downstream formation.

In some embodiments, provided technologies provides improved reagents compatibility. For example, as demonstrated in the present disclosure, provided technologies provide flexibility to use different reagent systems for oxidation, sulfurization and/or azide reactions, particularly for chirally controlled DMD oligonucleotide synthesis.

Among other things, the present disclosure provides DMD oligonucleotide compositions of high crude purity. In some embodiments, the present disclosure provides chirally controlled DMD oligonucleotide composition of high crude purity. In some embodiments, the present disclosure provides chirally controlled DMD oligonucleotide of high crude purity. In some embodiments, the present disclosure provides DMD oligonucleotide of high crude purity and/or high stereopurity.

### Support and Linkers

In some embodiments, oligonucleotides can be prepared in solution. In some embodiments, oligonucleotides can be prepared using a support. In some embodiments, oligonucleotides are prepared using a solid support. Suitable support that can be utilized in accordance with the present disclosure include, e.g., solid support described in U.S. Pat. Nos. 9,695,211, 9,605,019, 9,598,458, US 2013/0178612, US 20150211006, US 20170037399, WO 2017/015555, WO 2017/062862, WO 2017/160741, WO 2017/192664, WO 2017/192679, WO 2017/210647, WO 2018/223056. WO 2018/237194, WO 2019/055951, WO 2019/200185, and/or WO 2019/217784, the solid support of each of which is incorporated herein by reference.

In some embodiments, a linker moiety is utilized to connect an oligonucleotide chain to a support during synthesis. Suitable linkers are widely utilized in the art, and include those described in U.S. Pat. Nos. 9,695,211, 9,605,019, 9,598,458, US 2013/0178612, US 20150211006, US 20170037399, WO 2017/015555, WO 2017/062862, WO 2017/160741, WO 2017/192664, WO 2017/192679, WO 2017/210647, WO 2018/223056, WO 2018/237194, WO 2019/055951, WO 2019/200185, and/or WO 2019/217784, the linker of each of which is incorporated herein by reference

In some embodiments, the linking moiety is a succinamic acid linker, or a succinate linker (—CO—CH₂—CH₂—CO—), or an oxalyl linker (—CO—CO—). In some embodiments, the linking moiety and the nucleoside are bonded together through an ester bond. In some embodiments, a linking moiety and a nucleoside are bonded together through an amide bond. In some embodiments, a linking moiety connects a nucleoside to another nucleotide or nucleic acid. Suitable linkers are disclosed in, for example. Oligonucleotides And Analogues A Practical Approach, Ekstein, F. Ed., IRL Press, N.Y., 1991, Chapter 1 and Solid-Phase Supports for Oligonucleotide Synthesis, Pon, R. T., Curr. Prot. Nucleic Acid Chem., 2000, 3.1.1-3.1.28. In some embodiments, a universal linker (UnyLinker) is used to attached the oligonucleotide to the solid support (Ravikumar et al., Org. Process Res. Dev., 2008, 12 (3), 399-410). In some embodiments, other universal linkers are used (Pon, R. T., Curr. Prot. Nucleic Acid Chem., 2000, 3.1.1-3.1.28). In some embodiments, various orthogonal linkers (such as disulfide linkers) are used (Pon, R. T., Curr. Prot. Nucleic Acid Chem., 2000, 3.1.1-3.1.28).

Among other things, the present disclosure recognizes that a linker can be chosen or designed to be compatible with a set of reaction conditions employed in oligonucleotide synthesis. In some embodiments, to avoid degradation of oligonucleotides and to avoid desulfurization, auxiliary groups are selectively removed before de-protection. In some embodiments, DPSE group can selectively be removed by F ions. In some embodiments, the present disclosure provides linkers that are stable under a DPSE de-protection condition. e.g., 0.1M TBAF in MeCN, 0.5M HF—Et₃N in THF or MeCN, etc. In some embodiments, a provided linker is a linker as exemplified below:

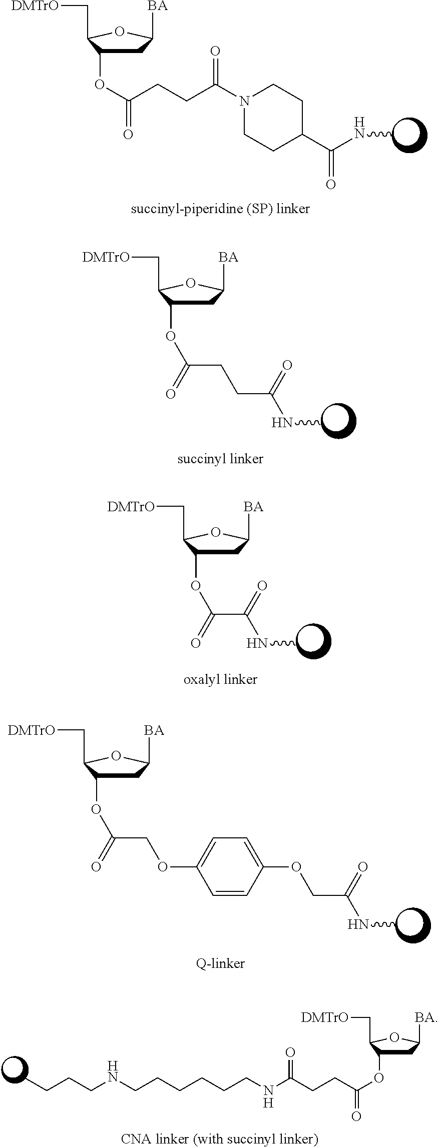

### Solvents

Syntheses of oligonucleotides are generally performed in aprotic organic solvents. In some embodiments, a solvent is a nitrile solvent such as, e.g., acetonitrile. In some embodiments, a solvent is a basic amine solvent such as, e.g., pyridine. In some embodiments, a solvent is an ethereal solvent such as, e.g., tetrahydrofuran. In some embodiments, a solvent is a halogenated hydrocarbon such as, e.g., dichloromethane. In some embodiments, a mixture of solvents is used. In certain embodiments a solvent is a mixture of any one or more of the above-described classes of solvents.

In some embodiments, when an aprotic organic solvent is not basic, a base is present in the reacting step. In some embodiments where a base is present, the base is an amine base such as, e.g., pyridine, quinoline, or N,N-dimethylaniline. Example other amine bases include pyrrolidine, piperidine, N-methyl pyrrolidine, pyridine, quinoline, N,N-dimethylaminopyridine (DMAP), or N,N-dimethylaniline.

In some embodiments, a base is other than an amine base.

In some embodiments, an aprotic organic solvent is anhydrous. In some embodiments, an anhydrous aprotic organic solvent is freshly distilled. In some embodiments, a freshly distilled anhydrous aprotic organic solvent is a basic amine solvent such as, e.g., pyridine. In some embodiments, a freshly distilled anhydrous aprotic organic solvent is an ethereal solvent such as, e.g., tetrahydrofuran. In some embodiments, a freshly distilled anhydrous aprotic organic solvent is a nitrile solvent such as, e.g., acetonitrile.

### Chiral Reagents/Chiral Auxiliaries

In some embodiments, chiral reagents (may also be referred to as chiral auxiliaries) are used to confer stereoselectivity in the production of chirally controlled oligonucleotides. Many chiral reagents, also referred to by those of skill in the art and herein as chiral auxiliaries, may be used in accordance with methods of the present disclosure. Examples of such chiral reagents are described herein and in U.S. Pat. Nos. 9,695,211, 9,605,019, 9,598,458, US 2013/0178612, US 20150211006. US 20170037399, WO 2017/015555, WO 2017/062862, WO 2017/160741, WO 2017/192664, WO 2017/192679, WO 2017/210647, WO 2018/098264, WO 2018/223056, WO 2018/237194, WO 2019/055951. WO 2019/200185, and/or WO 2019/217784, the chiral auxiliaries of each of which is incorporated by reference.

In some embodiments, a chiral reagent is a compound of Formula 3-AA:

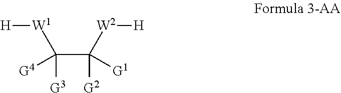

wherein each variable is independently as described herein.

In some embodiments of Formula 3-AA, W¹ and W² are independently —NG⁵-, —O—, or —S—; G¹, G², G³, G⁴, and G⁵ are independently hydrogen, or an optionally substituted group selected from aliphatic, alkyl, aralkyl, cycloalkyl, cycloalkylalkyl, heteroaliphatic, heterocyclyl, heteroaryl, or aryl; or two of G¹, G², G³, G⁴, and G⁵ are G⁶ (taken together to form an optionally substituted saturated, partially unsaturated or unsaturated carbocyclic or heteroatom-containing ring of up to about 20 ring atoms which is monocyclic or polycyclic, fused or unfused), and no more than four of G¹, G², G³, G⁴ and G⁵ are G⁶. Similarly to the compounds of Formula 3-I, any of G¹, G², G³, G⁴, or G⁵ are optionally substituted by oxo, thioxo, alkyl, alkenyl, alkynyl, heteroaryl, or aryl moieties. In some embodiments, such substitution induces stereoselectivity in chirally controlled oligonucleotide production. In some embodiments, a heteroatom-containing moiety, e.g., heteroaliphatic, heterocyclyl, heteroaryl, etc., has 1-5 heteroatoms. In some embodiments, the heteroatoms are selected from nitrogen, oxygen, sulfur and silicon. In some embodiments, at least one heteroatom is nitrogen. In some embodiments, aliphatic, alkyl, aralkyl, cycloalkyl, cycloalkylalkyl, heteroaliphatic, heterocyclyl, heteroaryl, or aryl groups have 1-20, 1-15, 1-10, 1-9, 1-8, 1-7 or 1-6 carbon atoms.

In some embodiments, W¹ is —NG⁵-O—. In some embodiments, W¹ is —NG⁵-O—, wherein the —O— is bonded to —H. In some embodiments, W¹ is —NG⁵-. In some embodiments, G⁵ and one of G³ and G⁴ are taken together to form an optionally substituted 3-10 membered ring having 0-3 heteroatoms in addition to the nitrogen atom of W¹. In some embodiments, G⁵ and G³ are taken together to form an optionally substituted 3-10 membered ring having 0-3 heteroatoms in addition to the nitrogen atom of W¹. In some embodiments, G⁵ and G⁴ are taken together to form an optionally substituted 3-10 membered ring having 0-3 heteroatoms in addition to the nitrogen atom of W¹. In some embodiments, a formed ring is an optionally substituted 4, 5, 6, 7, or 8 membered ring. In some embodiments, a formed ring is an optionally substituted 4-membered ring. In some embodiments, a formed ring is an optionally substituted 5-membered ring. In some embodiments, a formed ring is an optionally substituted 6-membered ring. In some embodiments, a formed ring is an optionally substituted 7-membered ring.

In some embodiments, a provided chiral reagent has the structure of

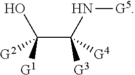

In some embodiments, a provided chiral reagent has the structure of

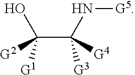

In some embodiments, a provided chiral reagent has the structure of

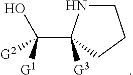

In some embodiments, a provided chiral reagent has the structure of

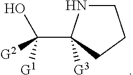

In some embodiments, a provided chiral reagent has the structure of

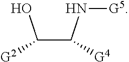

In some embodiments, a provided chiral reagent has the structure of

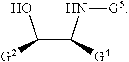

In some embodiments, a provided chiral reagent has the structure of

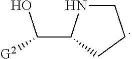

In some embodiments, a provided chiral reagent has the structure of

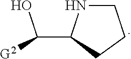

In some embodiments, W¹ is —NG⁵, W² is O, each of G¹ and G³ is independently hydrogen or an optionally substituted group selected from C₁₋₁₀ aliphatic, heterocyclyl, heteroaryl and aryl, G² is —C(R)₂Si(R)₃, and G⁴ and G⁵ are taken together to form an optionally substituted saturated, partially unsaturated or unsaturated heteroatom-containing ring of up to about 20 ring atoms which is monocyclic or polycyclic, fused or unfused. In some embodiments, each R is independently hydrogen, or an optionally substituted group selected from C₁-C₆ aliphatic, carbocyclyl, aryl, heteroaryl, and heterocyclyl. In some embodiments, G² is —C(R)₂Si(R)₃, wherein —C(R)₂— is optionally substituted —CH₂—, and each R of —Si(R)₃ is independently an optionally substituted group selected from C₁₋₁₀ in aliphatic, heterocyclyl, heteroaryl and aryl. In some embodiments, at least one R of —Si(R)₃ is independently optionally substituted C₁₋₁₀ alkyl. In some embodiments, at least one R of —Si(R)₃ is independently optionally substituted phenyl. In some embodiments, one R of —Si(R)₃ is independently optionally substituted phenyl, and each of the other two R is independently optionally substituted C₁₋₁₀ alkyl. In some embodiments, one R of —Si(R)₃ is independently optionally substituted C₁₋₁₀ alkyl, and each of the other two R is independently optionally substituted phenyl. In some embodiments, G² is optionally substituted —CH₂Si(Ph)(Me)₂. In some embodiments, G² is optionally substituted —CH₂Si(Me)(Ph)₂. In some embodiments, G² is —CH₂Si(Me)(Ph)₂. In some embodiments, G⁴ and G⁵ are taken together to form an optionally substituted saturated 5-6 membered ring containing one nitrogen atom (to which G⁵ is attached). In some embodiments, G⁴ and G⁵ are taken together to form an optionally substituted saturated 5-membered ring containing one nitrogen atom. In some embodiments. G¹ is hydrogen. In some embodiments. G³ is hydrogen. In some embodiments, both G¹ and G³ are hydrogen.

In some embodiments, W¹ is —NG⁵, W² is O, each of G¹ and G³ is independently R¹, G² is —R¹, and G⁴ and G⁵ are taken together to form an optionally substituted saturated, partially unsaturated or unsaturated heteroatom-containing ring of up to about 20 ring atoms which is monocyclic or polycyclic, fused or unfused, wherein R¹ is an optionally substituted group selected from C₁₋₂₀ aliphatic, C₁₋₂₀ aliphatic having 1-5 heteroatoms, C₆₋₂₀ aryl, C₅₋₂₀ heteroaryl having 1-5 heteroatoms and combinations thereof (e.g., aliphatic-aryl/heteroaryl, heteroaliphatic-aryl/heteroaryl). In some embodiments, each of G¹ and G³ is independently R. In some embodiments, each of G¹ and G³ is independently —H. In some embodiments, G² is connected to the rest of the molecule through a carbon atom, and the carbon atom is substituted with one or more electron-withdrawing groups. In some embodiments, G² is methyl substituted with one or more electron-withdrawing groups. In some embodiments, (G² is methyl substituted with one and no more than one electron-withdrawing group. In some embodiments, G² is methyl substituted with two or more electron-withdrawing groups. Among other things, a chiral auxiliary having G² comprising an electron-withdrawing group can be readily removed by a base (base-labile, e.g., under an anhydrous condition substantially free of water; in many instances, preferably before oligonucleotides comprising internucleotidic linkages comprising such chiral auxiliaries are exposed to conditions/reagent systems comprising a substantial amount of water, particular in the presence of a base(e.g., cleavage conditions/reagent systems using NH₄OH)) and provides various advantages as described herein, e.g., high crude purity, high yield, high stereoselectivity, more simplified operation, fewer steps, further reduced manufacture cost, and/or more simplified downstream formulation (e.g., low amount of salt(s) after cleavage), etc. In some embodiments, as described in the Examples, such auxiliaries may provide alternative or additional chemical compatibility with other functional and/or protection groups. In some embodiments, as demonstrated in the Examples, base-labile chiral auxiliaries are particularly useful for construction of chirally controlled non-negatively charged internucleotidic linkages (e.g., neutral internucleotidic linkages such as n001); in some instances, as demonstrated in the Examples, they can provide significantly improved yield and/or crude purity with high stereoselectivity, e.g., when utilized with removal using a base under an anhydrous condition. In some embodiments, such a chiral auxiliary is bonded to a linkage phosphorus via an oxygen atom (e.g., which corresponds to a —OH group in a corresponding chiral auxiliary compound), the carbon atom in the chiral auxiliary to which the oxygen is bonded (the alpha carbon) also bonds to —H (in addition to other groups; in some embodiments, a secondary carbon), and the next carbon atom (the beta carbon) in the chiral auxiliary is boned to one or two electron-withdrawing groups. In some embodiments, —W²—H is —OH. In some embodiments, G¹ is —H. In some embodiments, G² comprises one or two electron-withdrawing groups or can otherwise facilitate remove of the chiral auxiliary by a base. In some embodiments, G¹ is —H, G² comprises one or two electron-withdrawing groups, —W²—H is —OH. In some embodiments, G¹ is —H, G² comprises one or two electron-withdrawing groups, —W²—H is —OH, —W¹—H is —NG⁵-H, and one of G³ and G⁴ is taken together with G⁵ to form with their intervening atoms a ring as described herein (e.g., an optionally substituted 3-20 membered monocyclic, bicyclic or polycyclic ring having in addition to the nitrogen atom to which G⁵ is on, 0-5 heteroatoms (e.g., an optionally substituted 3, 4, 5, or 6-membered monocyclic saturated ring having in addition to the nitrogen atom to which G⁵ is on no other heteroatoms)).

As appreciated by those skilled in the art, various electron-withdrawing groups are known in the art and can be utilized in accordance with the present disclosure. In some embodiments, an electronic-withdrawing group comprises and/or is connected to the carbon atom through, e.g., —S(O)—, —S(O)₂—, —P(O)(R¹)—, —P(S)R¹—, or —C(O)—. In some embodiments, an electron-withdrawing group is —CN, —NO₂, halogen, —C(O)R¹, —C(O)OR′, —C(O)N(R′)₂, —S(O)R¹, —S(O)₂R¹, —P(W)(R¹)₂, —P(O)(R¹)₂, —P(O)(OR′)₂, or —P(S)(R¹)₂. In some embodiments, an electron-withdrawing group is aryl or heteroaryl, e.g., phenyl, substituted with one or more of —CN, —NO₂, halogen, —C(O)R¹, —C(O)OR′, —C(O)N(R′)₂, —S(O)R¹, —S(O)₂R¹, —P(W)R¹)₂, —P(O)(R¹)₂, —P(O)(OR′)₂, or —P(S)(R¹)₂.

In some embodiments, G² is -L′-L″-R′, wherein L′ is —C(R)₂—or optionally substituted —CH₂—, and L″ is —P(O)(R′)—, —P(O)(R′)O—, —P(O)(OR′)—, —P(O)(OR′)O—, —P(O)[N(R′)]—, —P(O)[N(R′)]O—, —P(O)[N(R′)][N(R′)]—, —P(S)(R′)—, —S(O)₂—, —S(O)₂—, —S(O)₂O—, —S(O)—, —C(O)—, —C(O)N(R′)—, or —S—, wherein each R′ is independently R¹ as described herein. In some embodiments, L′ is —C(R)₂—. In some embodiments, L′ is optionally substituted —CH₂—.

In some embodiments, L′ is —C(R)₂—. In some embodiments, each R is independently hydrogen, or an optionally substituted group selected from C₁-C₆ aliphatic, carbocyclyl, aryl, heteroaryl, and heterocyclyl. In some embodiments, L′ is —CH₂—. In some embodiments, L″ is —P(O)(R′)—, —P(S)(R′)—, —S(O)₂—. In some embodiments, G² is -L′-C(O)N(R′)₂. In some embodiments, G² is -L′-P(O)(R′)₂. In some embodiments, G² is -L′-P(S)(R′)₂. In some embodiments, each R′ is independently optionally substituted aliphatic, heteroaliphatic, aryl, or heteroaryl as described in the present disclosure (e.g., those embodiments described for R). In some embodiments, each R′ is independently optionally substituted phenyl. In some embodiments, each R′ is independently optionally substituted phenyl wherein one or more substituents are independently selected from —CN, —OMe, —Cl, —Br, and —F. In some embodiments, each R′ is independently substituted phenyl wherein one or more substituents are independently selected from —CN, —OMe, —Cl, —Br, and —F. In some embodiments, each R′ is independently substituted phenyl wherein the substituents are independently selected from —CN, —OMe, —Cl, —Br, and —F. In some embodiments, each R′ is independently mono-substituted phenyl, wherein the substituent is independently selected from —CN, —OMe, —Cl, —Br, and —F. In some embodiments, two R′ are the same. In some embodiments, two R′ are different. In some embodiments, G² is -L′-S(O)R′. In some embodiments, G² is -L′-C(O)N(R′)₂. In some embodiments, G² is -L′-S(O)₂R′. In some embodiments, R′ is optionally substituted aliphatic, heteroaliphatic, aryl, or heteroaryl as described in the present disclosure (e.g., those embodiments described for R). In some embodiments, R′ is optionally substituted phenyl. In some embodiments, R′ is optionally substituted phenyl wherein one or more substituents are independently selected from —CN, —OMe, —Cl, —Br, and —F. In some embodiments, R′ is substituted phenyl wherein one or more substituents are independently selected from —CN, —OMe, —Cl, —Br, and —F. In some embodiments, R′ is substituted phenyl wherein each substituent is independently selected from —CN, —OMe, —Cl, —Br, and —F. In some embodiments, R′ is mono-substituted phenyl. In some embodiments, R′ is mono-substituted phenyl, wherein the substituent is independently selected from —CN, —OMe, —Cl, —Br, and —F. In some embodiments, a substituent is an electron-withdrawing group. In some embodiments, an electron-withdrawing group is —CN, —NO₂, halogen, —C(O)R¹, —C(O)OR′, —C(O)N(R′)₂, —S(O)R¹, —S(O)₂R¹, —P(W)(R¹)₂, —P(O)(R¹)₂, —P(O)(OR′)₂, or —P(S)(R¹)₂.

In some embodiments, G² is optionally substituted —CH₂-L″-R, wherein each of L″ and R is independently as described in the present disclosure. In some embodiments, G² is optionally substituted —CH(-L″-R)₂, wherein each of L″ and R is independently as described in the present disclosure. In some embodiments, G² is optionally substituted —CH(—S—R)₂. In some embodiments, G² is optionally substituted —CH₂—S—R In some embodiments, the two R groups are taken together with their intervening atoms to form a ring. In some embodiments, a formed ring is an optionally substituted 5, 6, 7-membered ring having 0-2 heteroatoms in addition to the intervening heteroatoms. In some embodiments, G² is optionally substituted

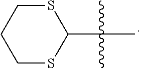 [SMILES: *C1SCCCS1]

In some embodiments, G² is

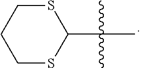 [SMILES: *C1SCCCS1]

In some embodiments, —S— may be converted to —S(O)— or —S(O)₂—, e.g., by oxidation, e.g., to facilitate removal by a base.

In some embodiments, G² is -L′-R′, wherein each variable is as described in the present disclosure. In some embodiments, G² is —CH₂—R′. In some embodiments, G² is —CH(R′)₂. In some embodiments, G² is —C(R′)₃. In some embodiments, R′ is optionally substituted aryl or heteroaryl. In some embodiments, R′ is substituted aryl or heteroaryl wherein one or more substituents are independently an electron-withdrawing group. In some embodiments, -L′- is optionally substituted —CH₂—, and R′ is R, wherein R is optionally substituted aryl or heteroaryl. In some embodiments, R is substituted aryl or heteroaryl wherein one or more substituents are independently an electron-withdrawing group. In some embodiments, R is substituted aryl or heteroaryl wherein each substituent is independently an electron-withdrawing group. In some embodiments, R is aryl or heteroaryl substituted with two or more substituents, wherein each substituent is independently an electron-withdrawing group. In some embodiments, an electron-withdrawing group is —CN, —NO₂, halogen, —C(O)R¹, —C(O)OR′, —C(O)N(R′)₂, —S(O)R¹, —S(O)₂R¹, —P(W)(R¹)₂, —P(O)(R¹)₂, —P(O)(OR′)₂, or —P(S)(R¹)₂. In some embodiments, R′ is

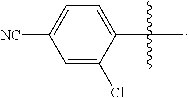 [SMILES: *c1ccc(C#N)cc1Cl]

In some embodiments, R′ is p-NO₂Ph-. In some embodiments, R′ is

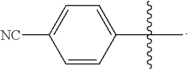 [SMILES: *c1ccc(C#N)cc1]

In some embodiments, R′ is

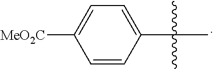 [SMILES: *c1ccc(C(=O)OC)cc1]

In some embodiments, R′ is

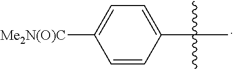

In some embodiments, R′ is

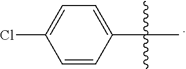 [SMILES: *c1ccc(Cl)cc1]

In some embodiments, R′ is

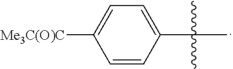

In some embodiments, G² is

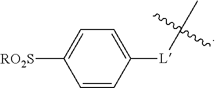

In some embodiments, R′ is

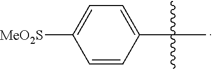 [SMILES: *c1ccc(S(C)(=O)=O)cc1]

In some embodiments, R′ is

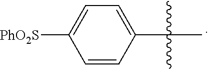

In some embodiments, R′ is 2,4,6-trichlorophenyl. In some embodiments, R′ is 2,4,6-trifluorophenyl. In some embodiments, G² is —CH(4-chlorophenyl)₂. In some embodiments, G² is —CH(R′)₂, wherein each R′ is

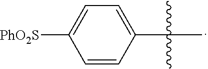

In some embodiments, G² is —CH(R′)₂, wherein each R′ is

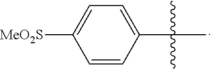 [SMILES: *c1ccc(S(C)(=O)=O)cc1]

In some embodiments, R′ is —C(O)R. In some embodiments, R′ is CH₃C(O)—.

In some embodiments, G² is -L′-S(O)₂R′, wherein each variable is as described in the present disclosure. In some embodiments, G² is —CH₂—S(O)₂R′. In some embodiments, G² is -L′-S(O)R′, wherein each variable is as described in the present disclosure. In some embodiments, G² is —CH₂—S(O)R′. In some embodiments, G² is -L′-C(O)₂R′, wherein each variable is as described in the present disclosure. In some embodiments, G² is —CH₂—C(O)₂R′. In some embodiments, G² is -L′-C(O)R′, wherein each variable is as described in the present disclosure. In some embodiments, G² is —CH₂—C(O)R′. In some embodiments, -L′- is optionally substituted —CH₂—, and R′ is R. In some embodiments, R is optionally substituted aryl or heteroaryl. In some embodiments, R is optionally substituted aliphatic. In some embodiments, R is optionally substituted heteroaliphatic. In some embodiments, R is optionally substituted heteroaryl. In some embodiments, R is optionally substituted aryl. In some embodiments, R is optionally substituted phenyl. In some embodiments, R is not phenyl, or mono-, di- or tri-substituted phenyl, wherein each substituent is selected from —NO₂, halogen, —CN, —C₁₋₃ alkyl, and C₁₋₃ alkyloxy. In some embodiments, R is substituted aryl or heteroaryl wherein one or more substituents are independently an electron-withdrawing group. In some embodiments, R is substituted aryl or heteroaryl wherein each substituent is independently an electron-withdrawing group. In some embodiments, R is aryl or heteroaryl substituted with two or more substituents, wherein each substituent is independently an electron-withdrawing group. In some embodiments, an electron-withdrawing group is —CN, —NO₂, halogen, —C(O)R¹, —C(O)OR′, —C(O)N(R′)₂, —S(O)R¹, —S(O)₂R¹, —P(W)(R¹)₂, —P(O)(R′)₂, —P(O)(OR′)₂, or —P(S)(R¹)₂. In some embodiments, R′ is phenyl. In some embodiments. R′ is substituted phenyl. In some embodiments, R′ is

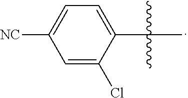 [SMILES: *c1ccc(C#N)cc1Cl]

In some embodiments, R′ is

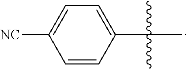 [SMILES: *c1ccc(C#N)cc1]

In some embodiments, R′ is

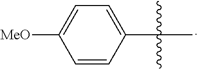 [SMILES: *c1ccc(OC)cc1]

In some embodiments, R′ is optionally substituted C₁₋₆ aliphatic. In some embodiments, R′ is t-butyl. In some embodiments, R′ is isopropyl. In some embodiments, R′ is methyl. In some embodiments, G² is —CH₂C(O)OMe. In some embodiments, G² is —CH₂C(O)Ph. In some embodiments, G² is —CH₂C(O)-tBu.

In some embodiments, G² is -L′-NO₂. In some embodiments, G² is —CH₂—NO₂. In some embodiments, G² is -L-S(O)₂N(R′)₂. In some embodiments, G² is —CH₂—S(O)₂N(R′)₂. In some embodiments, G² is -L′-S(O)₂NHR′. In some embodiments, G² is —CH₂—S(O)₂NHR′. In some embodiments, R′ is methyl. In some embodiments, G² is —CH₂—S(O)₂NH(CH₃). In some embodiments, R′ is —CH₂Ph. In some embodiments, G² is —CH₂—S(O)₂NH(CH₂Ph). In some embodiments, G² is —CH₂—S(O)₂N(CH₂Ph)₂. In some embodiments, R′ is phenyl. In some embodiments, G² is —CH₂—S(O)₂NHPh. In some embodiments, G² is —CH₂—S(O)₂N(CH₃)Ph. In some embodiments, G² is —CH₂—S(O)₂N(CH₃)₂. In some embodiments, G² is —CH₂—S(O)₂NH(CH₂Ph). In some embodiments, G² is —CH₂—S(O)₂NHPh. In some embodiments, G² is —CH₂—S(O)₂NH(CH₂Ph). In some embodiments, G² is —CH₂—S(O)₂N(CH₃)₂. In some embodiments, G² is —CH₂—S(O)₂N(CH₃)Ph. In some embodiments, G² is -L′-S(O)₂N(R′)(OR′). In some embodiments, G² is —CH₂—S(O)₂N(R′)(OR′). In some embodiments, each R′ is methyl. In some embodiments, G² is —CH₂—S(O)₂N(CH₃)(OCH₃). In some embodiments, G² is —CH₂—S(O)₂N(Ph)(OCH₃). In some embodiments, G² is —CH₂—S(O)₂N(CH₂Ph)(OCH₃). In some embodiments, G² is —CH₂—S(O)₂N(CH₂Ph)(OCH₃). In some embodiments, G² is -L′-S(O)₂OR′. In some embodiments, G² is —CH₂—S(O)₂OR′. In some embodiments, G² is —CH₂—S(O)₂OPh. In some embodiments, G² is —CH₂—S(O)₂OCH₃. In some embodiments, G² is —CH₂—S(O)₂OCH₂Ph.

In some embodiments, G² is -L′-P(O)(R′)₂. In some embodiments, G² is —CH₂—P(O)(R′)₂. In some embodiments. G² is -L′-P(O)[N(R′)₂]₂. In some embodiments, G² is —CH₂—P(O)[N(R′)₂]₂. In some embodiments, G² is -L′-P(O)[O(R′)₂]₂. In some embodiments, G² is —CH₂—P(O)[O(R′)₂]₂. In some embodiments, G² is -L′-P(O)(R′)[N(R′)₂]₂. In some embodiments, G² is —CH₂—P(O)(R′)[N(R′)₂]. In some embodiments, G² is -L′-P(O)(R′)[O(R′)]. In some embodiments, G² is —CH₂—P(O)(R′)[O(R′)]. In some embodiments, G² is -L′-P(O)(OR′)[N(R′)₂]. In some embodiments, G² is —CH₂—P(O)(OR′)[N(R′)₂]. In some embodiments, G² is -L′-C(O)N(R′)₂, wherein each variable is as described in the present disclosure. In some embodiments, G² is —CH₂—C(O)N(R′)₂. In some embodiments, each R′ is independently R. In some embodiments, one R′ is optionally substituted aliphatic, and one R is optionally substituted aryl. In some embodiments, one R′ is optionally substituted C₁₋₆ aliphatic, and one R is optionally substituted phenyl. In some embodiments, each R′ is independently optionally substituted C₁₋₆ aliphatic. In some embodiments, G² is —CH₂—P(O)(CH₃)Ph. In some embodiments, G² is —CH₂—P(O)(CH₃)₂. In some embodiments, G² is —CH₂—P(O)(Ph)₂. In some embodiments, G² is —CH₂—P(O)(OCH₃)₂. In some embodiments, G² is —CH₂—P(O)(CH₂Ph)₂. In some embodiments, G² is —CH₂—P(O)[N(CH₃)Ph]₂. In some embodiments, G² is —CH₂—P(O)[N(CH₃)₂]. In some embodiments, G² is —CH₂—P(O)[N(CH₂Ph)₂]². In some embodiments, G² is —CH₂—P(O)(OCH₃)₂. In some embodiments, G² is —CH₂—P(O)(OPh)₂.

In some embodiments, G² is -L′-SR′. In some embodiments, G² is —CH₂—SR′. In some embodiments, R′ is optionally substituted phenyl. In some embodiments, R′ is phenyl.

In some embodiments, a provided chiral reagent has the structure of

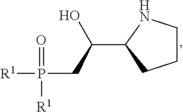

wherein each R¹ is independently as described in the present disclosure. In some embodiments, a provided chiral reagent has the structure of

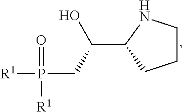

wherein each R¹ is independently as described in the present disclosure. In some embodiments, each R¹ is independently R as described in the present disclosure. In some embodiments, each R¹ is independently R, wherein R is optionally substituted aliphatic, aryl, heteroaliphatic, or heteroaryl as described in the present disclosure. In some embodiments, each R¹ is phenyl. In some embodiments, R¹ is -L-R′. In some embodiments, R¹ is -L-R′, wherein L is —O—, —S—, or —N(R′). In some embodiments, a provided chiral reagent has the structure of

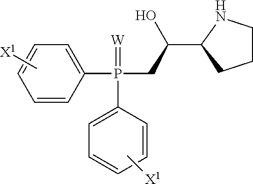

wherein each X¹ is independently —H, an electron-withdrawing group, —NO₂, —CN, —OR, —Cl, —Br, or —F, and W is O or S. In some embodiments, a provided chiral reagent has the structure of

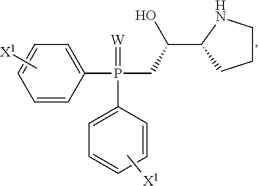

wherein each X¹ is independently —H, an electron-withdrawing group, —NO₂, —CN, —OR, —Cl, —Br, or —F, and W is O or S. In some embodiments, each X¹ is independently —CN, —OR, —Cl, —Br, or —F, wherein R is not —H. In some embodiments, R is optionally substituted C₁₋₆ aliphatic. In some embodiments, R is optionally substituted C₁₋₆ alkyl. In some embodiments, R is —CH₃. In some embodiments, one or more X¹ are independently electron-withdrawing groups (e.g., —CN, —NO₂, halogen, —C(O)R¹, —C(O)OR′, —C(O)N(R′)₂, —S(O)R¹, —S(O)₂R¹, —P(W)(R¹)₂, —P(O)(R¹)₂, —P(O)(OR′)₂, —P(S)(R¹)₂, etc.).

In some embodiments, a provided chiral reagent has the structure of

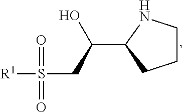

wherein R¹ is as described in the present disclosure. In some embodiments, a provided chiral reagent has the structure of

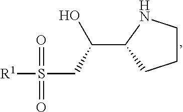

wherein R¹ is as described in the present disclosure. In some embodiments, R¹ is R as described in the present disclosure. In some embodiments, R¹ is R, wherein R is optionally substituted aliphatic, aryl, heteroaliphatic, or heteroaryl as described in the present disclosure. In some embodiments. R¹ is -L-R′. In some embodiments, R¹ is -L-R′, wherein L is —O—, —S—, or —N(R′). In some embodiments, a provided chiral reagent has the structure of

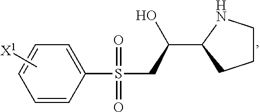

wherein X¹ is —H, an electron-withdrawing group, —NO₂. —CN, —OR, —Cl, —Br, or —F, and W is O or S. In some embodiments, a provided chiral reagent has the structure of

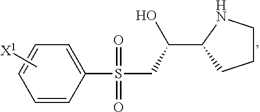

wherein X¹ is —H, an electron-withdrawing group, —NO₂, —CN, —OR, —Cl, —Br, or —F, and W is O or S. In some embodiments, X¹ is —CN, —OR, —Cl, —Br, or —F, wherein R is not —H. In some embodiments, R is optionally substituted C₁₋₆ aliphatic. In some embodiments, R is optionally substituted C₁₋₆ alkyl. In some embodiments, R is —CH₃. In some embodiments. X¹ is an electron-withdrawing group (e.g., —CN, —NO₂, halogen, —C(O)R¹, —C(O)OR′, —C(O)N(R′)₂, —S(O)R¹, —S(O)₂R¹, —P(W)(R¹)₂, —P(O)(R¹)₂, —P(O)(OR′)₂, —P(S)(R¹)₂, etc.). In some embodiments, X¹ is an electron-withdrawing group that is not —CN, —NO₂, or halogen. In some embodiments, X¹ is not —H, —CN, —NO₂, halogen, or C₁₋₃ alkyloxy.

In some embodiments, G² is —CH(R²¹)—CH(R²²)═C(R²³)(R²⁴), wherein each of R²¹, R²², R²³, and R²⁴ is independently R. In some embodiments, R²² and R²³ are both R, and the two R groups are taken together with their intervening atoms to form an optionally substituted aryl or heteroaryl ring as described herein. In some embodiments, one or more substituents are independently electron-withdrawing groups. In some embodiments, R²¹ and R²⁴ are both R, and the two R groups are taken together with their intervening atoms to form an optionally substituted ring as described herein. In some embodiments, R²¹ and R²⁴ are both R, and the two R groups are taken together with their intervening atoms to form an optionally substituted saturated or partially saturated ring as described herein. In some embodiments, R²² and R²³ are both R, and the two R groups are taken together with their intervening atoms to form an optionally substituted aryl or heteroaryl ring as described herein, and R²¹ and R²⁴ are both R, and the two R groups are taken together with their intervening atoms to form an optionally substituted partially saturated ring as described herein. In some embodiments, R²¹ is —H. In some embodiments, R²⁴ is —H. In some embodiments, G² is optionally substituted

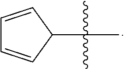 [SMILES: *C1C=CC=C1]

In some embodiments, G² is optionally substituted

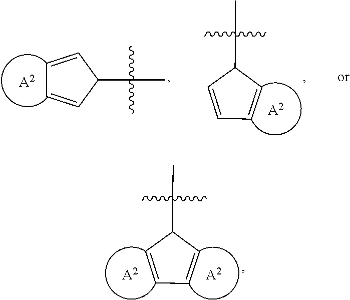

wherein each Ring A² is independently a 3-15 membered monocyclic, bicyclic or polycyclic ring as described herein. In some embodiments, Ring A² is an optionally substituted 5-10 membered monocyclic aryl or heteroaryl ring having 1-5 heteroatoms as described herein. In some embodiments, Ring A² is an optionally substituted phenyl ring as described herein. In some embodiments, In some embodiments, G² is optionally substituted

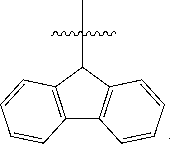 [SMILES: *C1c2ccccc2-c2ccccc21]

In some embodiments, G² is

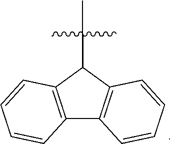 [SMILES: *C1c2ccccc2-c2ccccc21]

In some embodiments, G² is

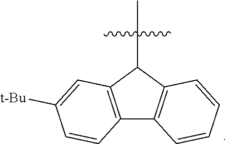 [SMILES: *C1c2ccccc2-c2ccc(C(C)(C)C)cc21]

In some embodiments, G² is

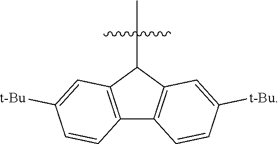 [SMILES: *C1c2cc(C(C)(C)C)ccc2-c2ccc(C(C)(C)C)cc21]

In some embodiments, a chiral auxiliary is a DPSE auxiliary. In some embodiments, a chiral auxiliary is a PSM auxiliary.

In some embodiments, when contacted with a base, a chiral auxiliary moiety, e.g., of an internucleotidic linkage, whose corresponding compound is a compound of Formula 3-1 or 3-AA may be released as an alkene, which has the same structure as a product formed by elimination of a water molecule from the corresponding compound (elimination of —W²—H═—OH and an alpha-H of G²). In some embodiments, such an alkene has the structure of (electron-withdrawing group)₂=C(R¹)-L-N(R⁵)(R⁶), (electron-withdrawing group)H═C(R¹)-L-N(R⁵)(R⁶), CH(-L″-R′)═C(R′)-L-N(R⁵)(R⁶) wherein the CH group is optionally substituted, or C^{x}═C(R¹)-L-N(R⁵)(R⁶), wherein C^{x} is optionally substituted

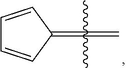 [SMILES: *=C1C=CC=C1]

and may be optionally fused with one or more optionally substituted rings, and each other variable is independently as described herein. In some embodiments, C^{x} is optionally substituted

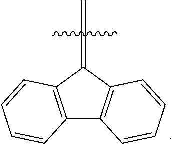 [SMILES: *=C1c2ccccc2-c2ccccc21]

In some embodiments, C^{x} is

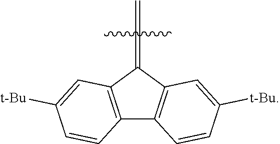 [SMILES: *=C1c2cc(C(C)(C)C)ccc2-c2ccc(C(C)(C)C)cc21]

In some embodiments, such an alkene is

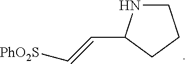

In some embodiments, such an alkene is

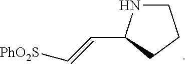

In some embodiments, such an alkene is

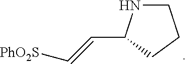

In some embodiments, a chiral reagent is an aminoalcohol. In some embodiments, a chiral reagent is an aminothiol. In some embodiments, a chiral reagent is an aminophenol. In some embodiments, a chiral reagent is (S)- and (R)-2-methylamino-1-phenylethanol, (1R, 2S)-ephedrine, or (1R, 2S)-2-methylamino-1,2-diphenylethanol.

In some embodiments of the disclosure, a chiral reagent is a compound of one of the following formulae:

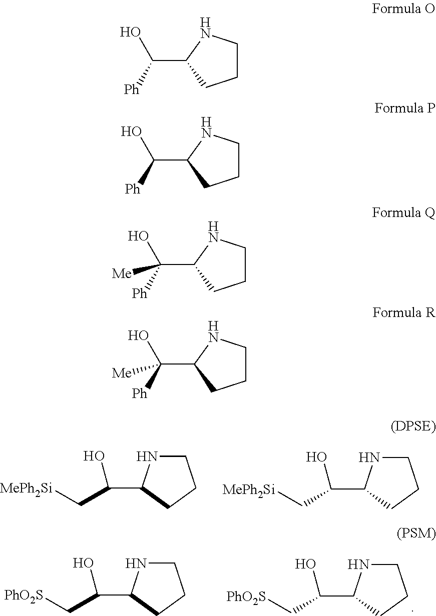

As appreciated by those skilled in the art, chiral reagents are typically stereopure or substantially stereopure, and are typically utilized as a single stereoisomer substantially free of other stereoisomers. In some embodiments, compounds of the present disclosure are stereopure or substantially stereopure.

As demonstrated herein, when used for preparing a chiral internucleotidic linkage, to obtain stereoselectivity generally stereochemically pure chiral reagents are utilized. Among other things, the present disclosure provides stereochemically pure chiral reagents, including those having structures described.

The choice of chiral reagent, for example, the isomer represented by Formula Q or its stereoisomer, Formula R, permits specific control of chirality at a linkage phosphorus. Thus, either an Rp or Sp configuration can be selected in each synthetic cycle, permitting control of the overall three dimensional structure of a chirally controlled DMD oligonucleotide. In some embodiments, a chirally controlled DMD oligonucleotide has all Rp stereocenters. In some embodiments of the disclosure, a chirally controlled DMD oligonucleotide has all Sp stereocenters. In some embodiments of the disclosure, each linkage phosphorus in the chirally controlled DMD oligonucleotide is independently Rp or Sp. In some embodiments of the disclosure, each linkage phosphorus in the chirally controlled DMD oligonucleotide is independently Rp or Sp, and at least one is Rp and at least one is Sp. In some embodiments, the selection of Rp and Sp centers is made to confer a specific three dimensional superstructure to a chirally controlled DMD oligonucleotide. Examples of such selections are described in further detail herein.

In some embodiments, a provided DMD oligonucleotide comprise a chiral auxiliary moiety, e.g., in an internucleotidic linkage. In some embodiments, a chiral auxiliary is connected to a linkage phosphorus. In some embodiments, a chiral auxiliary is connected to a linkage phosphorus through W². In some embodiments, a chiral auxiliary is connected to a linkage phosphorus through W², wherein W² is O. Optionally, W¹, e.g., when W¹ is —NG⁵-, is capped during DMD oligonucleotide synthesis. In some embodiments, W¹ in a chiral auxiliary in a DMD oligonucleotide is capped, e.g., by a capping reagent during DMD oligonucleotide synthesis. In some embodiments, W¹ may be purposeful capped to modulate DMD oligonucleotide property. In some embodiments, W¹ is capped with —R¹. In some embodiments, R¹ is —C(O)R′. In some embodiments, R′ is optionally substituted C₁₋₆ aliphatic. In some embodiments, R′ is methyl.

In some embodiments, a chiral reagent for use in accordance with the present disclosure is selected for its ability to be removed at a particular step in the above-depicted cycle. For example, in some embodiments it is desirable to remove a chiral reagent during the step of modifying the linkage phosphorus. In some embodiments, it is desirable to remove a chiral reagent before the step of modifying the linkage phosphorus. In some embodiments, it is desirable to remove a chiral reagent after the step of modifying the linkage phosphorus. In some embodiments, it is desirable to remove a chiral reagent after a first coupling step has occurred but before a second coupling step has occurred, such that a chiral reagent is not present on the growing DMD oligonucleotide during the second coupling (and likewise for additional subsequent coupling steps). In some embodiments, a chiral reagent is removed during the “deblock” reaction that occurs after modification of the linkage phosphorus but before a subsequent cycle begins. Example methods and reagents for removal are described herein.

In some embodiments, removal of chiral auxiliary is achieved when performing the modification and/or deblocking step, as illustrated in Scheme I. It can be beneficial to combine chiral auxiliary removal together with other transformations, such as modification and deblocking. A person of ordinary skill in the art would appreciate that the saved steps/transformation could improve the overall efficiency of synthesis, for instance, with respect to yield and product purity, especially for longer DMD oligonucleotides. One example wherein the chiral auxiliary is removed during modification and/or deblocking is illustrated in Scheme I.

In some embodiments, a chiral reagent for use in accordance with methods of the present disclosure is characterized in that it is removable under certain conditions. For instance, in some embodiments, a chiral reagent is selected for its ability to be removed under acidic conditions. In certain embodiments, a chiral reagent is selected for its ability to be removed under mildly acidic conditions. In certain embodiments, a chiral reagent is selected for its ability to be removed by way of an E1 elimination reaction (e.g., removal occurs due to the formation of a cation intermediate on the chiral reagent under acidic conditions, causing the chiral reagent to cleave from the DMD oligonucleotide). In some embodiments, a chiral reagent is characterized in that it has a structure recognized as being able to accommodate or facilitate an E1 elimination reaction. One of skill in the relevant arts will appreciate which structures would be envisaged as being prone toward undergoing such elimination reactions.

In some embodiments, a chiral reagent is selected for its ability to be removed with a nucleophile. In some embodiments, a chiral reagent is selected for its ability to be removed with an amine nucleophile. In some embodiments, a chiral reagent is selected for its ability to be removed with a nucleophile other than an amine.

In some embodiments, a chiral reagent is selected for its ability to be removed with a base. In some embodiments, a chiral reagent is selected for its ability to be removed with an amine. In some embodiments, a chiral reagent is selected for its ability to be removed with a base other than an amine.

In some embodiments, chirally pure phosphoramidites comprising chiral auxiliaries may be isolated before use. In some embodiments, chirally pure phosphoramidites comprising chiral auxiliaries may be used without isolation—in some embodiments, they may be used directly after formation.

### Activation

As appreciated by those skilled in the art, DMD oligonucleotide preparation may use various conditions, reagents, etc. to active a reaction component, e.g., during phosphoramidite preparation, during one or more steps during in the cycles, during post-cycle cleavage/deprotection, etc. Various technologies for activation can be utilized in accordance with the present disclosure, including but not limited to those described in U.S. Pat. No. 9,695,211. U.S. Pat. Nos. 9,605,019, 9,598,458, US 2013/0178612, US 20150211006, US 20170037399, WO 2017/015555, WO 2017/062862, WO 2017/160741, WO 2017/192664, WO 2017/192679, WO 2017/210647, WO 2018/223056, WO 2018/237194, WO 2019/055951. WO 2019/200185, and/or WO 2019/217784, the activation technologies of each of which are incorporated by reference. Certain activation technologies, e.g., reagents, conditions, methods, etc. are illustrated in the Examples.

### Coupling

In some embodiments, cycles of the present disclosure comprise stereoselective condensation/coupling steps to form chirally controlled internucleotidic linkages. For condensation, often an activating reagent is used, such as 4,5-dicyanoimidazole (DCI), 4,5-dichloroimidazole, 1-phenylimidazolium triflate (PhIMT), benzimidazolium triflate (BIT), benztriazole, 3-nitro-1,2,4-triazole (NT), tetrazole, 5-ethylthiotetrazole (ETT), 5-benzylthiotetrazole (BTT), 5-(4-nitrophenyl)tetrazole, N-cyanomethylpyrrolidinium triflate (CMPT), N-cyanomethylpiperidinium triflate, N-cyanomethyldimethylammonium triflate, etc. Suitable conditions and reagents, including chiral phosphoramidites, include those described in U.S. Pat. Nos. 9,695,211, 9,605,019, 9,598,458, US 2013/0178612, US 20150211006, US 20170037399, WO 2017/015555, WO 2017/062862, WO 2017/160741, WO 2017/192664, WO 2017/192679, WO 2017/210647, WO 2018/223056, WO 2018/237194. WO 2019/055951, WO 2019/200185, and/or WO 2019/217784, the condensation reagents, conditions and methods of each of which are incorporated by reference. Certain coupling technologies, e.g., reagents, conditions, methods, etc. are illustrated in the Examples.

In some embodiments, a chiral phosphoramidite for coupling has the structure of

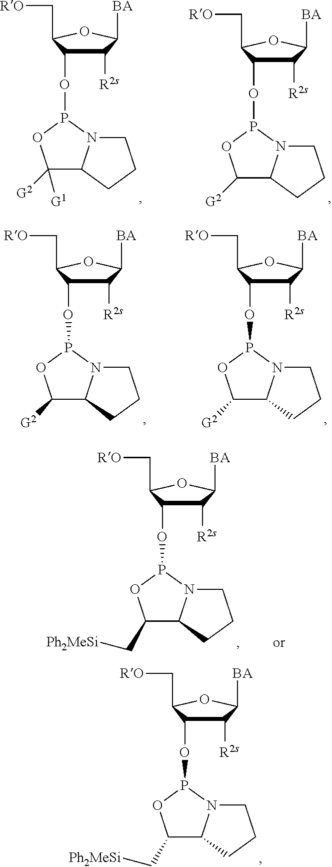

wherein R^{2s} is —H, —F, or —OR, and each other variable is independently as described in the present disclosure. In some embodiments, G¹ or G² comprises an electron-withdrawing group as described in the present disclosure. In some embodiments, a chiral phosphoramidite for coupling has the structure of

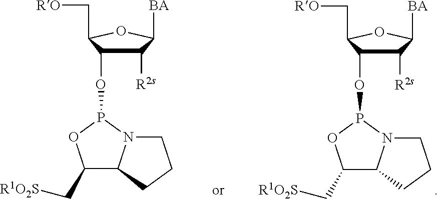

wherein each variable is independently as described in the present disclosure. In some embodiments, R¹ is R′ as described in the present disclosure. In some embodiments, R¹ is R as described in the present disclosure. In some embodiments, R is optionally substituted phenyl as described in the present disclosure. In some embodiments, R is phenyl. In some embodiments, R is optionally substituted C₁₋₆ aliphatic as described in the present disclosure. In some embodiments, R is optionally substituted C₁₋₆ alkyl as described in the present disclosure. For example, in some embodiments, R is methyl; in some embodiments, R is isopropyl; in some embodiments, R is t-butyl; etc. In some embodiments, R′ is a 5′-blocking group in oligonucleotide synthesis, e.g., DMTr. In some embodiments, BA is an optionally protected nucleobase as described herein. In some embodiments, BA is optionally substituted A, T, G. C. U or a tautomer thereof. In some embodiments, BA is a protected nucleobase. In some embodiments, BA is optionally substituted protected A, T, G, C, U or a tautomer thereof. In some embodiments, R′ is a protection group. In some embodiments, R′ is DMTr. In some embodiments, R^{2s} is —H, —F, or —OMe. In some embodiments, R^{2s} is —H. In some embodiments, R^{2s} is —F. In some embodiments, R^{2s} is —OMe.

In some embodiments, an internucleotidic linkage formed in a coupling step comprising,

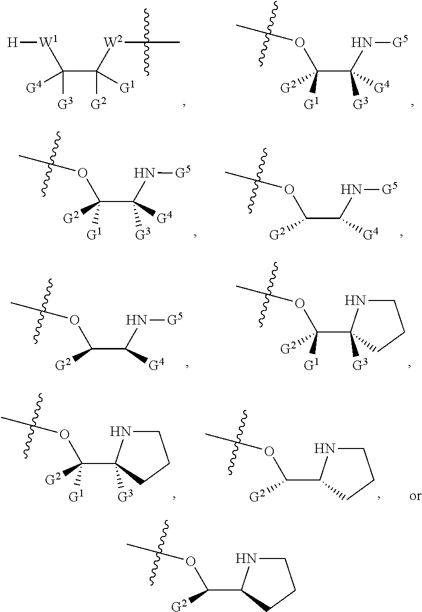

bonded to the linkage phosphorus, wherein each variable is independently in accordance with the present disclosure.

In some embodiments, a coupling forms an internucleotidic linkage with a stereoselectivity of 80%, 85%, 90%, 91%, 92%, 93%, 94%, 95%, 96%, 97%, 98%, 99% or more. In some embodiments, the stereoselectivity is 85% or more. In some embodiments, the stereoselectivity is 85% or more. In some embodiments, the stereoselectivity is 90% or more. In some embodiments, the stereoselectivity is 91% or more. In some embodiments, the stereoselectivity is 92% or more. In some embodiments, the stereoselectivity is 93% or more. In some embodiments, the stereoselectivity is 94% or more. In some embodiments, the stereoselectivity is 95% or more. In some embodiments, the stereoselectivity is 96% or more. In some embodiments, the stereoselectivity is 97% or more. In some embodiments, the stereoselectivity is 98% or more. In some embodiments, the stereoselectivity is 99% or more.

### Capping

If the final nucleic acid is larger than a dimer, the unreacted —OH moiety is generally capped with a blocking/capping group. Chiral auxiliaries in oligonucleotides may also be capped with a blocking group to form a capped condensed intermediate. Suitable capping technologies (e.g., reagents, conditions, etc.) include those described in U.S. Pat. Nos. 9,695,211, 9,605,019, 9,598,458, US 2013/0178612, US 20150211006, US 20170037399. WO 2017/015555, WO 2017/062862, WO 2017/160741, WO 2017/192664, WO 2017/192679, WO 2017/210647, WO 2018/223056, WO 2018/237194, WO 2019/055951, WO 2019/200185, and/or WO 2019/217784, the capping technologies of each of which are incorporated by reference. In some embodiments, a capping reagent is a carboxylic acid or a derivate thereof. In some embodiments, a capping reagent is R′COOH. In some embodiments, a capping step introduces R′COO— to unreacted 5′-OH group and/or amino groups in chiral auxiliaries. In some embodiments, a cycle may comprise two or more capping steps. In some embodiments, a cycle comprises a first capping before modification of a coupling product (e.g., converting P(III) to P(V)), and another capping after modification of a coupling product. In some embodiments, a first capping is performed under an amidation condition, e.g., which comprises an acylating reagent (e.g., an anhydride having the structure of (RC(O))₂O, (e.g., Ac₂O)) and a base (e.g., 2,6-lutidine). In some embodiments, a first capping caps an amino group, e.g., that of a chiral auxiliary in an internucleotidic linkage. In some embodiments, an internucleotidic linkage formed in a capping step comprises,

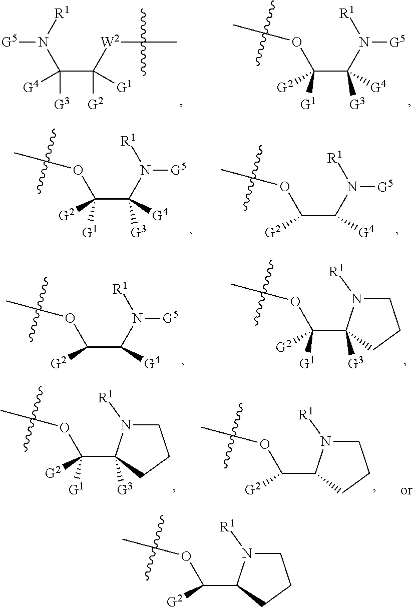

wherein each variable is independently in accordance with the present disclosure. In some embodiments, R¹ is R—C(O)—. In some embodiments, R is CH₃—. In some embodiments, each chirally controlled coupling (e.g., using a chiral auxiliary) is followed with a first capping. Typically, cycles for non-chirally controlled coupling using traditional phosphoramidite to construct natural phosphate linkages do not contain a first capping. In some embodiments, a second capping is performed, e.g., under an esterification condition (e.g., capping conditions of traditional phosphoramidite oligonucleotide synthesis) wherein free 5′-OH are capped.

Certain capping technologies, e.g., reagents, conditions, methods, etc. are illustrated in the Examples.

### Modifying

In some embodiments, an internucleotidic linkage wherein its linkage phosphorus exists as P(III) is modified to form another modified internucleotidic linkage. In many embodiments. P(III) is modified by reaction with an electrophile. Various types of reactions suitable for P(III) may be utilized in accordance with the present disclosure. Suitable modifying technologies (e.g., reagents (e.g., sulfurization reagent, oxidation reagent, etc.), conditions, etc.) include those described in U.S. Pat. Nos. 9,695,211, 9,605,019, 9,598,458, US 2013/0178612, US 20150211006, US 20170037399, WO 2017/015555. WO 2017/062862, WO 2017/160741, WO 2017/192664, WO 2017/192679, WO 2017/210647, WO 2018/223056, WO 2018/237194, WO 2019/055951, WO 2019/200185, and/or WO 2019/217784, the modifying technologies of each of which are incorporated by reference.

In some embodiments, as illustrated in the Examples, the present disclosure provides modifying reagents for introducing non-negatively charged internucleotidic linkages including neutral internucleotidic linkages.

In some embodiments, modifying is within a cycle. In some embodiments, modifying can be outside of a cycle. For example, in some embodiments, one or more modifying steps can be performed after the DMD oligonucleotide chain has been reached to introduce modifications simultaneously at one or more internucleotidic linkages and/or other locations.

In some embodiments, modifying comprises use of click chemistry, e.g., wherein an alkyne group of a DMD oligonucleotide, e.g., of an internucleotidic linkage, is reacted with an azide. Various reagents and conditions for click chemistry can be utilized in accordance with the present disclosure. In some embodiments, an azide has the structure of R¹—N₃, wherein R¹ is as described in the present disclosure. In some embodiments, R¹ is optionally substituted C₁₋₆ alkyl. In some embodiments, R¹ is isopropyl.

In some embodiments, as demonstrated in the examples, a P(III) linkage can be converted into a non-negatively charged internucleotidic linkage by reacting the P(III) linkage with an azide or an azido imidazolinium salt (e.g., a compound comprising

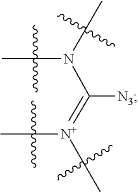 [SMILES: *N(*)C(N=[N+]=[N-])=[N+](*)*]

in some embodiments, referred to as an azide reaction) under suitable conditions. In some embodiments, an azido imidazolinium salt is a salt of PF₆⁻. In some embodiments, an azido imidazolinium salt is a salt of

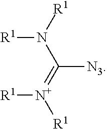

In some embodiments, a useful reagent is a salt of

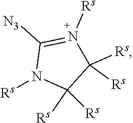

wherein each R^{s} is independently R¹. In some embodiments, a useful reagent is a salt of

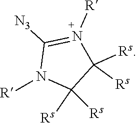

Such reagents comprising nitrogen cations also contain counter anions (e.g., Q⁻ as described in the present disclosure), which are widely known in the art and are contained in various chemical reagents. In some embodiments, a useful reagent is Q⁺Q⁻, wherein Q⁺ is

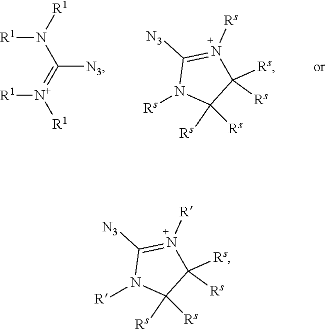

and Q⁻ is a counter anion. In some embodiments, Q⁺ is

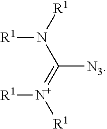

In some embodiments, Q⁺ is

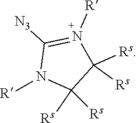

In some embodiments, Q⁺ is

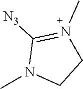

As appreciated by those skilled in the art, in a compound having the structure of Q⁺Q⁻, typically the number of positive charges in Q⁺ equals the number of negative charges in Q⁻. In some embodiments, Q⁺ is a monovalent cation and Q⁻ is a monovalent anion. In some embodiments, Q⁻ is F⁻, Cl⁻, Br⁻, BF₄⁻, PF₆⁻, TfO⁻, Tf₂N⁻, AsF₆⁻, ClO₄⁻, or SbF₆⁻. In some embodiments, Q⁻ is PF₆⁻. Those skilled in the art readily appreciate that many other types of counter anions are available and can be utilized in accordance with the present disclosure. In some embodiments, an azido imidazolinium salt is 2-azido-1,3-dimethylimidazolinium hexafluorophosphate.

In some embodiments, a P(III) linkage is reacted with an electrophile having the structure of R-G^{Z}, wherein R is as described in the present disclosure, and G^{Z} is a leaving group, e.g., —Cl, —Br, —I, —OTf, —Oms, —OTosyl, etc. In some embodiments, R is —CH₃. In some embodiments, R is —CH₂CH₃. In some embodiments, R is —CH₂CH₂CH₃. In some embodiments, R is —CH₂OCH₃. In some embodiments, R is CH₃CH₂OCH₂—. In some embodiments, R is PhCH₂OCH₂—. In some embodiments, R is HC≡C—CH₂—. In some embodiments. R is H₃C—C≡C—CH₂—. In some embodiments. R is CH₂═CHCH₂—. In some embodiments, R is CH₃SCH₂—. In some embodiments, R is —CH₂COOCH₃. In some embodiments, R is —CH₂COOCH₂CH₃. In some embodiments, R is —CH₂CONHCH₃.

In some embodiments, after a modifying step, a P(III) linkage phosphorus is converted into a P(V) internucleotidic linkage. In some embodiments, a P(III) linkage phosphorus is converted into a P(V) internucleotidic linkage, and all groups bounded to the linkage phosphorus remain unchanged. In some embodiments, a linkage phosphorus is converted from P into P(═O). In some embodiments, a linkage phosphorus is converted from P into P(═S). In some embodiments, a linkage phosphorus is converted from P into P(═N-L-R⁵). In some embodiments, a linkage phosphorus is converted from P into

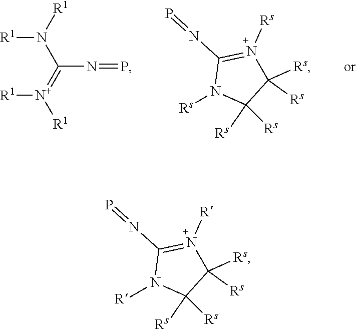

wherein each variable is independently as described in the present disclosure. In some embodiments, P is converted into

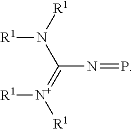

In some embodiments, P is converted into

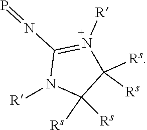

In some embodiments, P is converted into

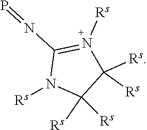

In some embodiments, P is converted into

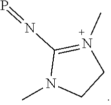

As appreciated by those skilled in the art, for each cation there typically exists a counter anion so that the total number of positive charges equals the total number of negative charges in a system (e.g., compound, composition, etc.). In some embodiments, a counter anion is Q⁻ as described in the present disclosure (e.g., F⁻, Cl⁻, Br⁻, BF₄⁻, PF₆⁻, TfO⁻, Tf₂N⁻, AsF₆⁻, ClO₄⁻, SbF₆⁻, etc.).

In some embodiments, such an internucleotidic linkage is chirally controlled. In some embodiments, all such internucleotidic linkages are chirally controlled. In some embodiments, linkage phosphorus of at least one of such internucleotidic linkages is Rp. In some embodiments, linkage phosphorus of at least one of such internucleotidic linkages is Sp. In some embodiments, linkage phosphorus of at least one of such internucleotidic linkages is Rp, and linkage phosphorus of at least one of such internucleotidic linkages is Sp. In some embodiments, DMD oligonucleotides of the present disclosure comprises one or more (e.g., 1-5, 1-10, 1-15, 1-20, 1-25, 1-30, 1-40, 1-50, 1, 2, 3, 4, 5, 6, 7, 8, 9, 10, 11, 12, 13, 14, 15, 16, 17, 18, 19, 20, 21, 22, 23, 24, 25, etc.) such internucleotidic linkages. In some embodiments, such DMD oligonucleotide further comprise one or more other types of internucleotidic linkages, e.g., one or more natural phosphate linkages, and/or one or more phosphorothioate internucleotidic linkages (e.g., in some embodiments, one or more of which are independently chirally controlled; in some embodiments, each of which is independently chirally controlled; in some embodiments, at least one is Rp; in some embodiments, at least one is Sp; in some embodiments, at least one is Rp and at least one is Sp; etc.) In some embodiments, such DMD oligonucleotides are stereopure (substantially free of other stereoisomers). In some embodiments, the present disclosure provides chirally controlled DMD oligonucleotide compositions of such DMD oligonucleotides. In some embodiments, the present disclosure provides chirally pure DMD oligonucleotide compositions of such DMD oligonucleotides.

In some embodiments, modifying proceeds with a stereoselectivity of 80%, 85%, 90%, 91%, 92%, 93%, 94%, 95%, 96%, 97%, 98%, 99% or more. In some embodiments, the stereoselectivity is 85% or more. In some embodiments, the stereoselectivity is 85% or more. In some embodiments, the stereoselectivity is 90% or more. In some embodiments, the stereoselectivity is 91% or more. In some embodiments, the stereoselectivity is 92% or more. In some embodiments, the stereoselectivity is 93% or more. In some embodiments, the stereoselectivity is 94% or more. In some embodiments, the stereoselectivity is 95% or more. In some embodiments, the stereoselectivity is 96% or more. In some embodiments, the stereoselectivity is 97% or more. In some embodiments, the stereoselectivity is 98% or more. In some embodiments, the stereoselectivity is 99% or more. In some embodiments, modifying is stereospecific.

### Deblocking

In some embodiments, a cycle comprises a cycle step. In some embodiments, the 5′ hydroxyl group of the growing DMD oligonucleotide is blocked (i.e., protected) and must be deblocked in order to subsequently react with a nucleoside coupling partner.

In some embodiments, acidification is used to remove a blocking group. Suitable deblocking technologies (e.g., reagents, conditions, etc.) include those described in U.S. Pat. Nos. 9,695,211, 9,605,019, 9,598,458, US 2013/0178612, US 20150211006, US 20170037399, WO 2017/015555, WO 2017/062862, WO 2017/160741, WO 2017/192664, WO 2017/192679, WO 2017/210647, WO 2018/223056, WO 2018/237194, WO 2019/055951, WO 2019/200185, and/or WO 2019/217784, the deblocking technologies of each of which are incorporated by reference. Certain deblocking technologies, e.g., reagents, conditions, methods, etc. are illustrated in the Examples.

### Cleavage and Deprotection

At certain stage, e.g., after the desired DMD oligonucleotide lengths have been achieved, cleavage and/or deprotection are performed to deprotect blocked nucleobases etc. and cleave the DMD oligonucleotide products from support. In some embodiments, cleavage and deprotection are performed separately. In some embodiments, cleavage and deprotection are performed in one step, or in two or more steps but without separation of products in between. In some embodiments, cleavage and/or deprotection utilizes basic conditions and elevated temperature. In some embodiments, for certain chiral auxiliaries, a fluoride condition is required (e.g., TBAF, HF-ET₃N, etc., optionally with additional base). Suitable cleavage and deprotection technologies (e.g., reagents, conditions, etc.) include those described in U.S. Pat. Nos. 9,695,211, 9,605,019, 9,598,458, US 2013/0178612, US 20150211006, US 20170037399, WO 2017/015555, WO 2017/062862, WO 2017/160741, WO 2017/192664, WO 2017/192679, WO 2017/210647, WO 2018/223056, WO 2018/237194, WO 2019/055951, WO 2019/200185, and/or WO 2019/217784, the cleavage and deprotection technologies of each of which are incorporated by reference. Certain cleavage and deprotection technologies, e.g., reagents, conditions, methods, etc. are illustrated in the Examples.

In some embodiments, certain chiral auxiliaries are removed under basic conditions. In some embodiments, DMD oligonucleotides are contacted with a base, e.g., an amine having the structure of N(R)₃, to remove certain chiral auxiliaries (e.g., those comprising an electronic-withdrawing group in G² as described in the present disclosure). In some embodiments, a base is NHR₂. In some embodiments, each R is independently optionally substituted C₁₋₆ aliphatic. In some embodiments, each R is independently optionally substituted C₁₋₆ alkyl. In some embodiments, an amine is DEA. In some embodiments, an amine is TEA. In some embodiments, an amine is provided as a solution, e.g., an acetonitrile solution. In some embodiments, such contact is performed under anhydrous conditions. In some embodiments, such a contact is performed immediately after desired DMD oligonucleotide lengths are achieved (e.g., first step post synthesis cycles). In some embodiments, such a contact is performed before removal of chiral auxiliaries and/or protection groups and/or cleavage of DMD oligonucleotides from a solid support. In some embodiments, contact with a base may remove cyanoethyl groups utilized in standard DMD oligonucleotide synthesis, providing an natural phosphate linkage which may exist in a salt form (with the cation being, e.g., an ammonium salt).

### Cycles

Suitable cycles for preparing DMD oligonucleotides of the present disclosure include those described in U.S. Pat. Nos. 9,695,211, 9,605,019, 9,598,458, US 2013/0178612, US 20150211006, US 20170037399, WO 2017/015555, WO 2017/062862, WO 2017/160741, WO 2017/192664, WO 2017/192679, WO 2017/210647 (e.g., Schemes I, I-b, I-c, I-d, I-c, I-f, etc.), WO 2018/223056, WO 2018/237194, WO 2019/055951, WO 2019/200185, and/or WO 2019/217784, the cycles of each of which are incorporated by reference. For example, in some embodiments, an example cycle is Scheme I-f. Certain cycles are illustrated in the Examples (e.g., for preparation of natural phosphate linkages, utilizing other chiral auxiliaries, etc.).

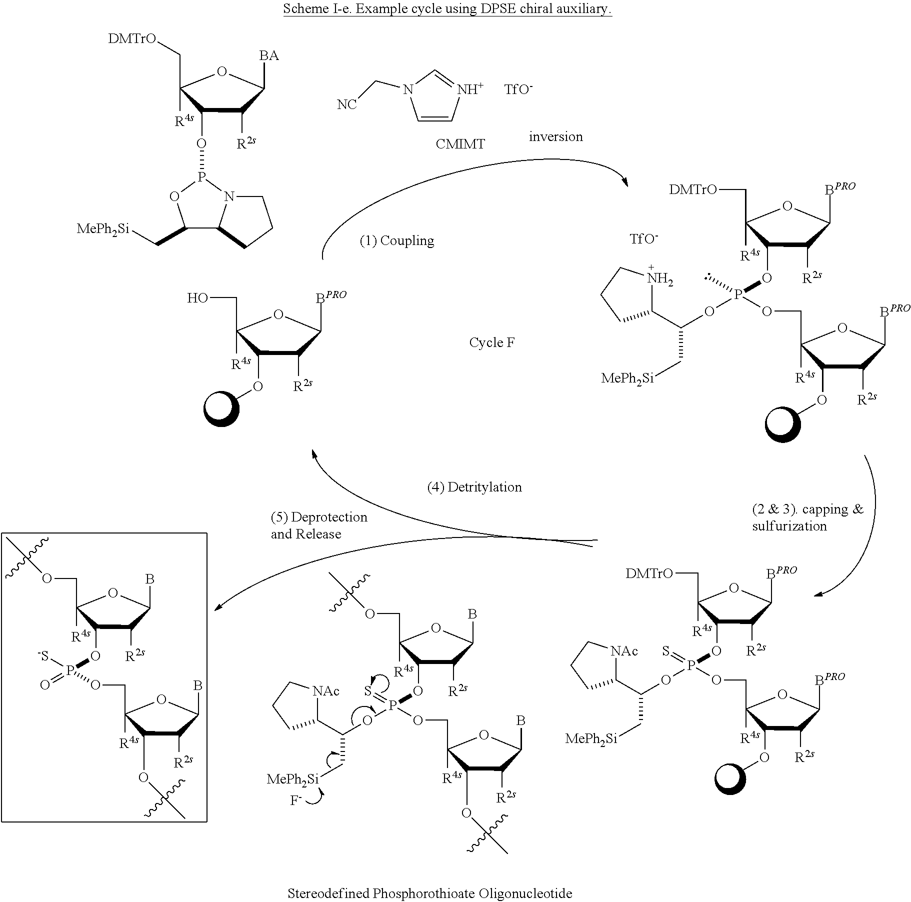

In some embodiments, R^{2s} is H or —OR¹, wherein R¹ is not hydrogen. In some embodiments, R^{2s} is H or —OR¹, wherein R¹ is optionally substituted C₁₋₆ alkyl. In some embodiments, R^{2s} is H. In some embodiments, R^{2s} is —OMe. In some embodiments, R^{2s} is —OCH₂CH₂OCH₃. In some embodiments, R^{2s} is —F. In some embodiments, R^{4s} is —H. In some embodiments, R^{4s} and R^{2s} are taken together to form a bridge -L-O—as described in the present disclosure. In some embodiments, the —O—is connected to the carbon at the 2′ position. In some embodiments, L is —CH₂—. In some embodiments, L is —CH(Me)-. In some embodiments, L is —(R)—CH(Me)-. In some embodiments, L is —(S)—CH(Me)-.

### Purification and Characterization

Various purification and/or characterization technologies (methods, instruments, protocols, etc.) can be utilized to purify and/or characterize DMD oligonucleotides and DMD oligonucleotide compositions in accordance with the present disclosure. In some embodiments, purification is performed using various types of HPLC/UPLC technologies. In some embodiments, characterization comprises MS, NMR, UV, etc. In some embodiments, purification and characterization may be performed together, e.g., HPLC-MS, UPLC-MS, etc. Example purification and characterization technologies include those described in U.S. Pat. Nos. 9,695,211, 9,605,019, 9,598,458, US 2013/0178612, US 20150211006, US 20170037399, WO 2017/015555, WO 2017/062862, WO 2017/160741, WO 2017/192664, WO 2017/192679, WO 2017/210647, WO 2018/223056, WO 2018/237194, WO 2019/055951, WO 2019/200185, and/or WO 2019/217784, the purification and characterization technologies of each of which are incorporated by reference.

In some embodiments, the present disclosure provides methods for preparing provided DMD oligonucleotide and DMD oligonucleotide compositions. In some embodiments, a provided method comprises providing a provided chiral reagent having the structure of formula 3-AA as described herein. In some embodiments, a provided method comprises providing a provided chiral reagent having the structure of

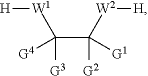

wherein W¹ is —NG⁵, W² is O, each of G¹ and G³ is independently hydrogen or an optionally substituted group selected from C₁₋₁₀ aliphatic, heterocyclyl, heteroaryl and aryl, G² is —C(R)₂Si(R)₃ or —C(R)₂SO₂R¹, and G⁴ and G⁵ are taken together to form an optionally substituted saturated, partially unsaturated or unsaturated heteroatom-containing ring of up to about 20 ring atoms which is monocyclic or polycyclic, fused or unfused, wherein each R is independently hydrogen, or an optionally substituted group selected from C₁-C₆ aliphatic, carbocyclyl, aryl, heteroaryl, and heterocyclyl.

In some embodiments, a provided chiral reagent has the structure of

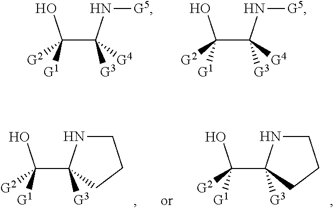

wherein each variable is independently as described in the present disclosure. In some embodiments, a provided methods comprises providing a phosphoramidite comprising a moiety from a chiral reagent having the structure of

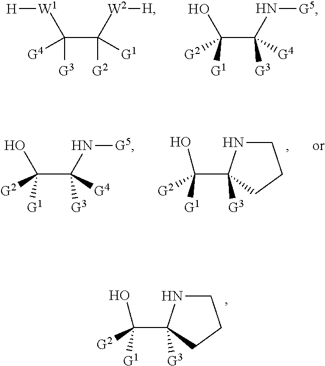

wherein —W¹H and —W²H, or the hydroxyl and amino groups, form bonds with the phosphorus atom of the phosphoramidite. In some embodiments, —W¹H and —W²H, or the hydroxyl and amino groups, form bonds with the phosphorus atom of the phosphoramidite, e.g., in

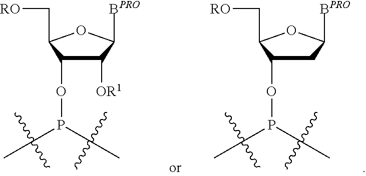

In some embodiments, a phosphoramidite has the structure of

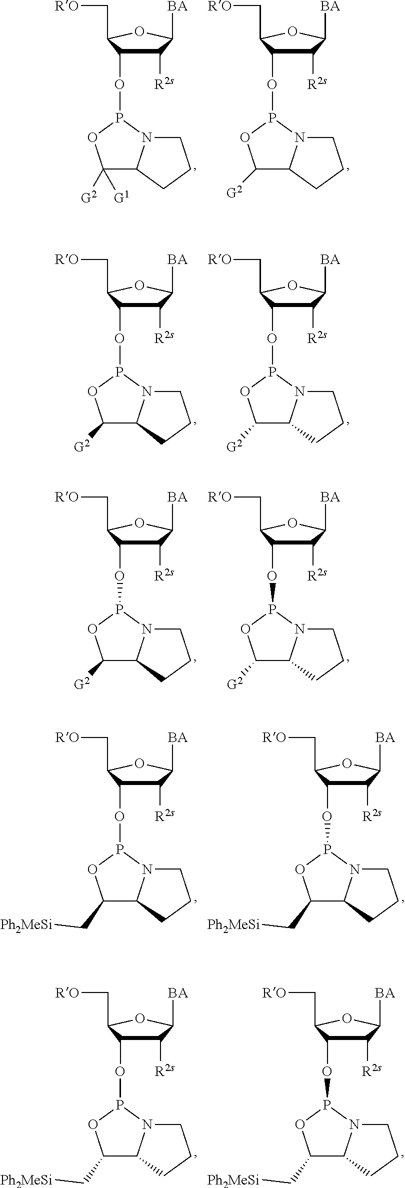

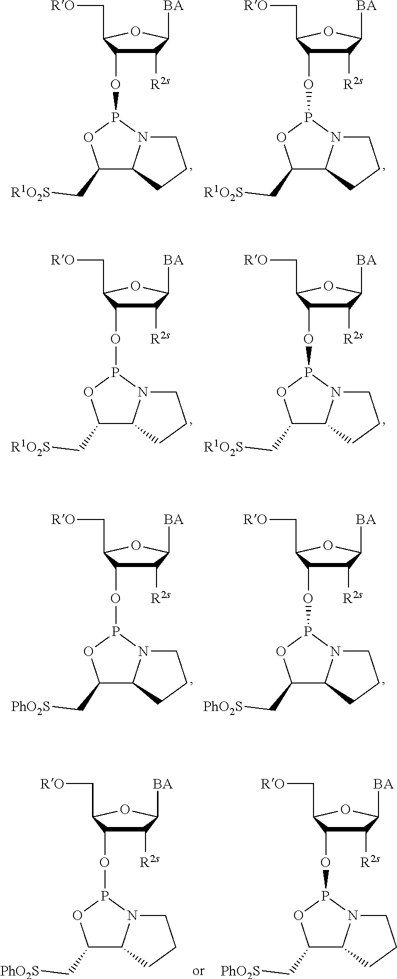

wherein each other variable is independently as described in the present disclosure. In some embodiments, R^{2s} is —H. In some embodiments, R^{2s} is —F. In some embodiments, R^{2s} is —OMe. In some embodiments, R′ is DMTr. In some embodiments, BA is optionally substituted A, T, C, G, U or an optionally substituted tautomer of A, T, C, G, or U.

In some embodiments. G² is —C(R)₂Si(R)₃, wherein —C(R)₂— is optionally substituted —CH₂—, and each R of —Si(R)₃ is independently an optionally substituted group selected from C₁₋₁₀ aliphatic, heterocyclyl, heteroaryl and aryl. In some embodiments, at least one R of —Si(R)₃ is independently optionally substituted C₁₋₁₀ alkyl. In some embodiments, at least one R of —Si(R)₃ is independently optionally substituted phenyl. In some embodiments, one R of —Si(R)₃ is independently optionally substituted phenyl, and each of the other two R is independently optionally substituted C₁₋₁₀ alkyl. In some embodiments, one R of —Si(R)₃ is independently optionally substituted C₁₋₁₀ alkyl, and each of the other two R is independently optionally substituted phenyl. In some embodiments, G² is optionally substituted —CH₂Si(Ph)(Me)₂. In some embodiments, G² is optionally substituted —CH₂Si(Me)(Ph)₂. In some embodiments, G² is —CH₂Si(Me)(Ph)₂. In some embodiments, G⁴ and G⁵ are taken together to form an optionally substituted saturated 5-6 membered ring containing one nitrogen atom (to which G⁵ is attached). In some embodiments, G⁴ and G⁵ are taken together to form an optionally substituted saturated 5-membered ring containing one nitrogen atom. In some embodiments, G¹ is hydrogen. In some embodiments, G³ is hydrogen. In some embodiments, both G¹ and G³ are hydrogen. In some embodiments, both G¹ and G³ are hydrogen, G² is —C(R)₂Si(R)₃, wherein —C(R)₂— is optionally substituted —CH₂—, and each R of —Si(R)₃ is independently an optionally substituted group selected from C₁₋₁₀ aliphatic, heterocyclyl, heteroaryl and aryl, and G⁴ and G⁵ are taken together to form an optionally substituted saturated 5-membered ring containing one nitrogen atom. In some embodiments, a provided method further comprises providing a fluoro-containing reagent. In some embodiments, a provided fluoro-containing reagent removes a chiral reagent, or a product formed from a chiral reagent, from oligonucleotides after synthesis. Various known fluoro-containing reagents, including those F⁻ sources for removing —SiR₃ groups, can be utilized in accordance with the present disclosure, for example, TBAF, HF₃-Et₃N etc. In some embodiments, a fluoro-containing reagent provides better results, for example, shorter treatment time, lower temperature, less de-sulfurization, etc, compared to traditional methods, such as concentrated ammonia. In some embodiments, for certain fluoro-containing reagent, the present disclosure provides linkers for improved results, for example, less cleavage of DMD oligonucleotides from support during removal of chiral reagent (or product formed therefrom during DMD oligonucleotide synthesis). In some embodiments, a provided linker is an SP linker. In some embodiments, the present disclosure demonstrated that a HF-base complex can be utilized, such as HF—NR₃, to control cleavage during removal of chiral reagent (or product formed therefrom during DMD oligonucleotide synthesis). In some embodiments, HF—NR₃ is HF-NEt₃. In some embodiments, HF—NR₃ enables use of traditional linkers, e.g., succinyl linker.

In some embodiments, as described herein, G² comprises an electron-withdrawing group, e.g., at its a position. In some embodiments, G² is methyl substituted with one or more electron-withdrawing groups. In some embodiments, an electronic-withdrawing group comprises and/or is connected to the carbon atom through, e.g., —S(O)—, —S(O)₂—, —P(O)(R¹)—, —P(S)R¹—, or —C(O)—. In some embodiments, an electron-withdrawing group is —CN, —NO₂, halogen, —C(O)R¹, —C(O)OR′, —C(O)N(R′)₂, —S(O)R¹, —S(O)₂R¹, —P(W)(R¹)₂, —P(O)(R¹)₂, —P(O)(OR′)₂, or —P(S)(R¹)₂. In some embodiments, an electron-withdrawing group is aryl or heteroaryl, e.g., phenyl, substituted with one or more of —CN, —NO₂, halogen, —C(O)R¹, —C(O)OR′, —C(O)N(R′)₂, —S(O)R¹, —S(O)₂R¹, —P(W)(R¹)₂, —P(O)(R¹)₂, —P(O)(OR′)₂, or —P(S)(R¹)₂. In some embodiments, G² is —CH₂S(O)R′. In some embodiments, G² is —CH₂S(O)₂R′. In some embodiments, G² is —CH₂P(O)(R′)₂. Additional example embodiments are described, e.g., as for chiral reagents/auxiliaries.

Confirmation that a stereocontrolled oligonucleotide (e.g., one prepared by a method described herein or in the art) comprises the intended stereocontrolled (chirally controlled) internucleotidic linkage can be performed using a variety of suitable technologies. A stereocontrolled (chirally controlled) oligonucleotide comprises at least one stereocontrolled internucleotidic linkage, which can be, e.g., a stereocontrolled internucleotidic linkage comprising a phosphorus, a stereocontrolled phosphorothioate internucleotidic linkage (PS) in the Rp configuration, a PS in the Sp configuration, etc. Useful technologies include, as non-limiting examples: NMR (e.g., ID (one-dimensional) and/or 2D (two-dimensional) ¹H-³¹P HETCOR (heteronuclear correlation spectroscopy)), HPLC, RP-HPLC, mass spectrometry, LC-MS, and/or stereospecific nucleases. In some embodiments, stereospecific nucleases include: benzonase, micrococcal nuclease, and svPDE (snake venomc phosphodiesterase), which are specific for internucleotidic linkages in the Rp configuration (e.g., a PS in the Rp configuration); and nuclease P1, mung bean nuclease, and nuclease Si, which are specific for internucleotidic linkages in the Sp configuration (e.g., a PS in the Sp configuration).

In some embodiments, the present disclosure pertains to a method of confirming or identifying the stereochemistry pattern of the backbone of an oligonucleotide, e.g., a DMD oligonucleotide and/or stereochemistry of particular internucleotidic linkages. In some embodiments, a DMD oligonucleotide comprises a stereocontrolled internucleotidic linkage comprising a phosphorus, a stereocontrolled phosphorothioate (PS) in the Rp configuration, or a PS in the Sp configuration. In some embodiments, a DMD oligonucleotide comprises at least one stereocontrolled internucleotidic linkage and at least one internucleotidic linkage which is not stereocontrolled. In some embodiments, a method comprises digestion of a DMD oligonucleotide with a stereospecific nuclease. In some embodiments, a stereospecific nuclease is selected from: benzonase, micrococcal nuclease, and svPDE (snake venom phosphodiesterase), which are specific for internucleotidic linkages in the Rp configuration (e.g., a PS in the Rp configuration); and nuclease P1, mung bean nuclease, and nuclease S1, which are specific for internucleotidic linkages in the Sp configuration (e.g., a PS in the Sp configuration). In some embodiments, a DMD oligonucleotide or fragments thereof produced by digestion with a stereospecific nuclease are analyzed. In some embodiments, a DMD oligonucleotide or fragments thereof(e.g., produced by digestion with a stereospecific nuclease) are analyzed by NMR. 1D (one-dimensional) and/or 2D (two-dimensional) ¹H-³¹P HETCOR (heteronuclear correlation spectroscopy), HPLC, RP-HPLC, mass spectrometry, LC-MS, UPLC, etc. In some embodiments, a DMD oligonucleotide or fragments thereof are compared with chemically synthesized fragments of the DMD oligonucleotide having a known pattern of stereochemistry.

Without wishing to be bound by any particular theory, the present disclosure notes that, in at least some cases, stereospecificity of a particular nuclease may be altered by a modification (e.g., 2′-modification) of a sugar, by a base sequence, or by a stereochemical context. For example, in some embodiments, benzonase and micrococcal nuclease, which are specific for Rp internucleotidic linkages, were both unable to cleave an isolated PS Rp internucleotidic linkage flanked by PS Sp internucleotidic linkages.

Various techniques and materials can be utilized. In some embodiments, the present disclosure provides useful combinations of technologies. For example, in some embodiments, stereochemistry of one or more particular internucleotidic linkages of a DMD oligonucleotide can be confirmed by digestion of the DMD oligonucleotide with a stereospecific nuclease and analysis of the resultant fragments (e.g., nuclease digestion products) by any of a variety of techniques (e.g., separation based on mass-to-charge ratio, NMR, HPLC, mass spectrometry, etc.). In some embodiments, stereochemistry of products of digesting a DMD oligonucleotide with a stereospecific nuclease can be confirmed by comparison (e.g., NMR, HPLC, mass spectrometry, etc.) with chemically synthesized fragments (e.g., dimers, trimers, tetramers, etc.) produced, e.g., via technologies that control stereochemistry.

In yet another example, a different DMD oligonucleotide was tested to confirm that the internucleotidic linkages were in the intended configurations. The DMD oligonucleotide is capable of skipping exon 51 of DMD; the majority of the nucleotides in the DMD oligonucleotide were 2′-F and the remainder were 2′-OMe; the majority of the internucleotidic linkages in the DMD oligonucleotide were PS in the Sp configuration and the remainder were PO. This DMD oligonucleotide was tested by digestion with stereospecific nucleases, and the resultant digestion fragments were analyzed (e.g., by LC-MS and by comparison with chemically synthesized fragments of known stereochemistry). The results confirmed that the DMD oligonucleotide had the intended pattern of stereocontrolled internucleotidic linkages.

In some embodiments, NMR is useful for characterization and/or confirming stereochemistry. In a set of example experiments, a set of DMD oligonucleotides comprising a stereocontrolled CpG motif were tested to confirm the intended stereochemistry of the CpG motif. Oligonucleotides of the set comprise a motif having the structure of pCpGp, wherein C is Cytosine, G is Guanine, and p is a phosphorothioate which is stereorandom or stereocontrolled (e.g., in the Rp or Sp configuration). For example, one DMD oligonucleotide comprises a pCpGp structure, wherein the pattern of stereochemistry of the phosphorothioates (e.g., the ppp) was RRR; in another DMD oligonucleotide, the pattern of stereochemistry of the ppp was RSS; in another DMD oligonucleotide, the pattern of stereochemistry of the ppp was RSR; etc. In the set, all possible patterns of stereochemistry of the ppp were represented. In the portion of the DMD oligonucleotide outside the pCpGp structure, all the internucleotidic linkages were PO; all nucleosides in the DMD oligonucleotides were 2′-deoxy. These various DMD oligonucleotides were tested in NMR, without digestion with a stereospecific nuclease, and distinctive patterns of peaks were observed, indicating that each PS which was Rp or Sp produced a unique peak, and confirming that the DMD oligonucleotides comprised stereocontrolled PS internucleotidic linkages of the intended stereochemistry.

Stereochemistry patterns of the internucleotidic linkages of various other stereocontrolled DMD oligonucleotides were confirmed, wherein the DMD oligonucleotides comprise a variety of chemical modifications and patterns of stereochemistry.

Technologies for Assessing, Detecting and/or Quantifying Oligonucleotides

In some embodiments, the present disclosure provides technologies for assessing, detecting and/or quantifying oligonucleotides. In some embodiments, the present disclosure pertains to a method of assessing oligonucleotide levels. e.g., in samples. In some embodiments, a method utilizes, is or comprises a hybridization enzyme-linked immunosorbent assay (HELISA).

In some embodiments, HELISA can be used to quantify oligonucleotides (which may be referred to as analytes for various samples). In some embodiments, test samples include, but are not limited to, plasma, cerebrospinal fluid, urine, and tissue homogenates (e.g., brain, spinal cord, liver, kidney, spleen, and other tissues). In some embodiments, provided technologies, e.g., HELISA, are useful for pharmacokinetic and toxicokinetic evaluation of oligonucleotides or oligonucleotide compositions, e.g., during development, clinical trials, post-approval, etc.

In some embodiments, the present disclosure provides a method, comprising
obtaining a capture probe oligonucleotide whose base sequence is or comprises a sequence that is complementary to a base sequence of a first oligonucleotide or a portion thereof;
contacting a capture probe oligonucleotide with a first oligonucleotide, and hybridizing the capture probe oligonucleotide with the first oligonucleotide;
obtaining a detection probe oligonucleotide whose base sequence is or comprises a sequence that is complementary to that of a portion of the capture probe oligonucleotide, wherein the base sequence of the portion of the capture probe oligonucleotide is not complementary to the base sequence of the first oligonucleotide:
contacting the detection probe oligonucleotide with the capture probe oligonucleotide, and hybridizing the detection probe oligonucleotide with the capture probe oligonucleotide which is hybridized with the first oligonucleotide;
covalently linking the first oligonucleotide and the detection probe oligonucleotide; and
removing detection probe oligonucleotides that are not covalently linked to the first oligonucleotide.

In some embodiments, provided methods are useful for detecting and/or quantifying a first oligonucleotide in a sample.

In some embodiments, a capture probe oligonucleotide comprises a tag. In some embodiments, a tag can be utilized to immobilize a capture probe oligonucleotide. In some embodiments, a tag is on a 3′-end of an oligonucleotide. In some embodiments, a tag is biotin.

In some embodiments, a detection probe oligonucleotide comprises a label for detection, quantification, etc. Useful label are widely available and can be utilized in accordance with the present disclosure. For example, in some embodiments, a label is an antigen which can be detected by an antibody, which can then be assessed through another assay (e.g., as in ELISA or technologies similarly thereto). In some embodiments, a label a fluorescent label. In some embodiments, a label is a radioactive label.

In some embodiments, oligonucleotides are covalently linked through, e.g., ligation. As appreciated by those skilled in the art, various ligation technology are available and can be utilized in accordance with the present disclosure.

In some embodiments, removing detection probe oligonucleotides that are not covalently linked to the first oligonucleotide comprises removing free detection probe oligonucleotides, and complexes that are not formed by a capture probe oligonucleotide and a ligation product formed by a first oligonucleotide and a detection probe oligonucleotide, wherein the capture probe oligonucleotide is complementary to the ligation product. In some embodiments, a capture probe oligonucleotide and a ligation product are of the same length, and are fully complementary to each other. In some embodiments, after removing, substantially all label are in complexes formed by ligation products and capture probe oligonucleotides. In some embodiments, removing are performed by contacting a system with a S1 nuclease, wherein single-stranded oligonucleotides and/or oligonucleotide strands with mismatches are digested.

In some embodiments, provided methods comprise detecting and/or quantifying detection probe oligonucleotides. As appreciated by those skilled in the art, various technologies can be utilized for detection and/or quantification, e.g., those in ELISA and/or similar technologies. In some embodiments, through detection/quantification of detect probe oligonucleotides, a first oligonucleotide is detected and/or quantified.

In some embodiments, a detection probe oligonucleotide is 9 nucleosides in length. In some embodiments, a first oligonucleotide is 20 nucleosides in length. In some embodiments, a capture probe oligonucleotide has a length which is the sum of a detection probe oligonucleotide and a first oligonucleotide (e.g., if a first oligonucleotide is a 20mer, a detection probe oligonucleotide is a 9mer, a capture probe oligonucleotide is a 29mer).

In some embodiments, the present disclosure provide a complex comprising a provided oligonucleotide and a capture probe oligonucleotide. In some embodiments, the present disclosure provides an oligonucleotide which is a ligation product of a provided oligonucleotide and a detection probe oligonucleotide. In some embodiments, the present disclosure provides a complex comprising a capture probe oligonucleotide and a ligation product oligonucleotide. In some embodiments, a capture probe oligonucleotide is immobilized, e.g., to a surface, a solid support, etc.

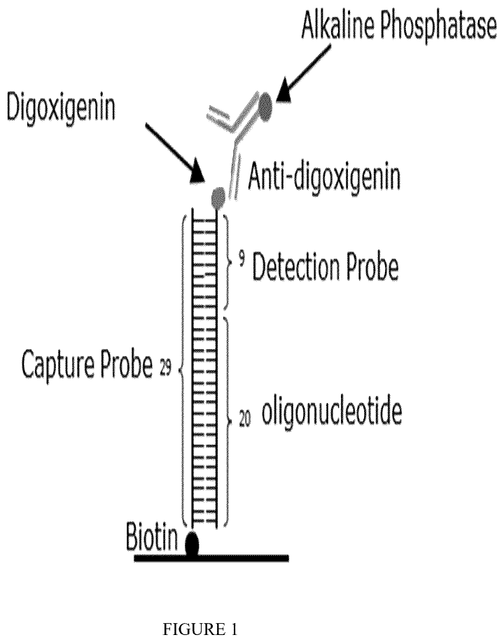

FIG. 1 describes a useful example. In FIG. 1, an assay employs a capture probe oligonucleotide that base pairs to an oligonucleotides, leaving an overhang on the 5′ end of the capture probe. The capture probe is covalently tagged with biotin on the 3′ end, which allows the capture probe: oligonucleotide complex to be pulled down on a plate pre-coated with molecules such as avidin, neutravidin, streptavidin, anti-biotin antibodies and other biotin binding molecules. A detection probe oligonucleotide, in this case a short 9-mer oligonucleotide with a covalent 3-detector tag, such as digoxigenin or others, is then added along with T4 DNA ligase. The detection probe oligonucleotide base pairs to the 5′ overhang on the capture probe. Base pairing of the detection probe to the capture probe provides a template for T4 DNA ligase to ligate the 3′ end of the oligonucleotide to the 5′ end of the detection probe. Among other things, ligation is performed so that, if desired, detection probe oligonucleotide remains hybridized to capture probes. After ligation, S1 nuclease is used to clip all unpaired capture probes from biotin or non-double-stranded complexes. As shown in FIG. 1, when ligation of the detection probe to the full-length parent oligonucleotide proceeds efficiently, a full 29-mer double-stranded complex is formed, which is resistant to cleavage and degradation by Si nuclease. In a situation where the detector tag is digoxigenin, addition of alkaline phosphatase (AP)-conjugated anti-digoxigenin antibody provides detection and/or quantification of the complex via addition of the AP substrate, whereby dephosphorylation by AP yields a fluorescent signal.

In some embodiments, it was observed that sensitivity and/or specificity may be impacted by covalent ligation of first oligonucleotides to the detection probe. In some embodiments, without covalent linking (e.g., ligation), a detection probe oligonucleotide may not remain bound to a capture probe until detection/quantification. Therefore, if T4 DNA ligase mediated ligation is not efficient, significant impairment of the signal will result. In some embodiments, covalent linking, e.g., ligation, of a first oligonucleotide can different a first oligonucleotide (an analyte) from its shorter metabolites (e.g., 3′ N-1 mer (e.g., if N is 20, 19-mer)). In some embodiments, shorter metabolite oligonucleotides are not long enough to template ligation, e.g., T4 DNA mediated ligation. In some embodiments, complexes comprising un-ligated detection probe oligonucleotides are removed. In some embodiments, such complexes are labile to degradation mediated by, e.g., Si nuclease. Various technologies can be utilized in accordance with the present disclosure to improve linking efficiency, e.g., ligation efficiency, and thus the overall detection/quantification efficiency. In some embodiments, polyethylene glycol (PEG) has been utilized to improve provided technologies, e.g., HELISA assay.

In some embodiments, provided technologies comprise utilization of a detergent, e.g., PEG, DMSO and/or betaine. In some embodiments, provided technologies comprise utilization of a detergent, e.g., PEG. In some embodiments, provided technologies comprise utilization of dimethylsulfoxide (DMSO). In some embodiments, provided technologies comprise utilization of betaine.

In some embodiments, it was observed that addition of 10-20% solutions of PEG (ranging from 2000 to 6000 average molecular weight), 5% dimethylsulfoxide (DMSO), and/or 0.5 M-1.5 M betaine enhanced overall signal with minimal impact on background signal. In some embodiments, signals were improved by about 10 fold, thereby significantly improving the sensitivity of the method. In some embodiments, for PEG, a dose-dependent increase in signal was observed when increasing the percentage of the polymer from 5-20%, equivalent across different average molecular weight PEG species (2000, 4000, and 6000) (Table 6).

TABLE 6
Concentration dependent effect of different PEG molecular weight species.
Data in this table are average background subtracted responses of duplicate measurements for conditions
indicated. A 20-mer oligonucleotide was utilized in the assay for assessment using suitable capture and
detection probe oligonucleotides; it comprises various nucleobase, sugar and internucleotidic linkage
modifications and each of its chiral internucleotidic linkage is independently chirally controlled.

| | | PEG Molecular Weight | | |
|---|---|---|---|---|
| Oligonucleotide | PEG Percentage | 2000 | 4000 | 6000 |
| 0.5 ng/mL | 0% | 23.25 | | |
| | 5% | 1.16 | −1.82 | 7.77 |
| | 10% | 247.06 | 228.36 | 287.64 |
| | 20% | 473.16 | 491.90 | 479.65 |
| 5 ng/mL | 0% | 348.29 | | |
| | 5% | 1318.61 | 1359.53 | 1454.66 |
| | 10% | 5095.92 | 4977.37 | 5647.40 |
| | 20% | 6538.09 | 6766.78 | 7238.19 |
| 50 ng/mL | 0% | 3983.78 | | |
| | 5% | 14661.62 | 15994.26 | 16711.34 |
| | 10% | 32132.79 | 32123.30 | 32665.46 |
| | 20% | 35261.80 | 35880.18 | 36638.60 |

In some embodiments, for betaine, a dose-dependent increase in signal was observed, e.g., when increasing the concentration of betaine from 0.1 M, 0.5 M to 1.5 M (Table 7). In some embodiments, for DMSO, 5% is sufficient to yield an increase in signal comparable to 10% PEG-6000 or 1.5 M betaine. In some embodiments, combination of 10% PEG-6000 with betaine or DMSO also provided increase in signal.

TABLE 7
Chemical additives improve signal responses, with or without inclusion of PEG.
Data in this table are average background subtracted responses of duplicate measurements
for conditions indicated. The same oligonucleotide was utilized for assessment as in Table 6.

| | No PEG-6000 | | | | | 10% PEG-6000 | | | | |
|---|---|---|---|---|---|---|---|---|---|---|
| Oligo- | | 5% | 0.1 M | 0.5 M | 1. 5 M | | 5% | 0.1 M | 0.5 M | 1.5 M |
| nucleotide | None | DMSO | Betaine | Betaine | Betaine | None | DMSO | Betaine | Betaine | Betaine |
| 0.8 ng/mL | 4.56 | 68.62 | 0.12 | 23.33 | 63.61 | 78.11 | 140.73 | 81.18 | 83.26 | 75.98 |
| 4 ng/mL | 180.98 | 1290.61 | 109.44 | 476.83 | 1317.92 | 1589.65 | 2461.54 | 1445.09 | 1535.48 | 1383.11 |
| 20 ng/mL | 1955.16 | 11290.71 | 1040.88 | 4475.63 | 10729.77 | 14453.51 | 16738.76 | 12272.30 | 14716.33 | 11991.78 |

Evaluation of PEG-6000 for various other oligonucleotides comprising various sequences and/or chemical modifications yielded similar sensitivity gains.

TABLE 8
PEG can improve signals. Various concentrations of a 20-mer oligonucleotide were utilized (can
be utilized as calibration curves; oligonucleotide for this Table (different from the oligonucleotide for
Tables 6 and 7) comprises various nucleobase, sugar and internucleotidic linkage modifications and each
of its chiral internucleotidic linkage is independently chirally controlled). Data in this table are average
background subtracted responses of duplicate measurements.

| Oligonucleotide | No PEG-6000 | 10% PEG-6000 |
|---|---|---|
| 5.00 | 3481.04 | 26267.22 |
| 2.50 | 2375.74 | 18706.35 |
| 1.25 | 1883.35 | 12769.13 |
| 0.625 | 2162.84 | 8071.38 |
| 0.313 | 946.97 | 5173.12 |
| 0.156 | 566.97 | 2426.78 |
| 0.0781 | 334.41 | 1469.73 |
| 0.0391 | 185.66 | 853.73 |
| 0.0195 | 139.88 | 372.95 |
| 0.00977 | 53.42 | 150.14 |

### Biological Applications, Example Use, and Dosing Regimens

As described herein, provided compositions and methods are useful for various purposes, e.g., those described in U.S. Pat. Nos. 9,695,211, 9,605,019, 9,598,458, US 2013/0178612, US 20150211006, US 20170037399, WO 2017/015555, WO 2017/062862, WO 2017/160741, WO 2017/192664, WO 2017/192679, and/or WO 2017/210647.

In some embodiments, provided technologies skip exon 51 in a target DMD transcript. A number of DMD oligonucleotides comprising various types of modified internucleotidic linkages, including many comprising non-negatively charged internucleotidic linkages (e.g., n001), which have various base sequences and/or target various nucleic acids (e.g., DMD transcripts of various genes) were prepared, and various useful properties, activities, and/or advantages were demonstrated.

In some embodiments, the present disclosure provides methods for modulating level of a DMD transcript or a product encoded thereby in a system, comprising administering an effective amount of a provided DMD oligonucleotide or a composition thereof. In some embodiments, the present disclosure provides methods for modulating level of a DMD transcript or a product encoded thereby in a system, comprising contacting the DMD transcript a provided DMD oligonucleotide or a composition thereof. In some embodiments, a system is an in vitro system. In some embodiments, a system is a cell. In some embodiments, a system is a tissue. In some embodiments, a system is an organ. In some embodiments, a system is an organism. In some embodiments, a system is a subject. In some embodiments, a system is a human. In some embodiments, modulating level of a DMD transcript decreases level of the DMD transcript. In some embodiments, modulating level of a DMD transcript increases level of the DMD transcript.

In some embodiments, the present disclosure provides methods for preventing or treating a condition, disease, or disorder associated with a nucleic acid sequence or a product encoded thereby, comprising administering to a subject suffering therefrom or susceptible thereto an effective amount of a provided DMD oligonucleotide or composition thereof, wherein the DMD oligonucleotide or composition thereof modulate level of a DMD transcript of the nucleic acid sequence. In some embodiments, a nucleic acid sequence is a gene. In some embodiments, modulating level of a DMD transcript decreases level of the DMD transcript. In some embodiments, modulating level of a DMD transcript increases level of the DMD transcript.

In some embodiments, change of the level of a modulated DMD transcript, e.g., through knock-down, exon skipping, etc., is at least 1.1, 1.2, 1.3, 1.4, 1.5, 2, 3, 4, 5, 6, 7, 8, 9, 10, 15, 20, 25, 30, 40, 50, 100, 200, 500, or 1000 fold.

In some embodiments, provided DMD oligonucleotides and DMD oligonucleotide compositions modulate splicing. In some embodiments, provided DMD oligonucleotides and DMD oligonucleotide compositions promote exon skipping, thereby produce a level of a DMD transcript which has increased beneficial functions that the DMD transcript prior to exon skipping. In some embodiments, a beneficial function is encoding a protein that has increased biological functions. In some embodiments, the present disclosure provides methods for modulating splicing, comprising administering to a splicing system a provided DMD oligonucleotide or DMD oligonucleotide composition, wherein splicing of at least one DMD transcript is altered (e.g., skipping of exon 51 is increased). In some embodiments, level of at least one splicing product is increased at least 1.1, 1.2, 1.3, 1.4, 1.5, 2, 3, 4, 5, 6, 7, 8, 9, 10, 15, 20, 25, 30, 40, 50, 100, 200, 500, or 1000 fold. In some embodiments, the present disclosure provides methods for modulating DMD splicing, comprising administering to a splicing system a provided DMD oligonucleotide or composition thereof.

In some embodiments, the present disclosure provides methods for preventing or treating DMD, comprising administering to a subject susceptible thereto or suffering therefrom a pharmaceutical composition comprising an effective amount of a provided DMD oligonucleotide or DMD oligonucleotide composition.

In some embodiments, provided compositions and methods provide improved splicing patterns of DMD transcripts compared to a reference pattern, which is a pattern from a reference condition selected from the group consisting of absence of the composition, presence of a reference composition, and combinations thereof. An improvement can be an improvement of any desired biological functions. In some embodiments, for example, in DMD, an improvement is production of an mRNA from which a dystrophin protein with improved biological activities is produced.

In some embodiments, particularly useful and effective are chirally controlled DMD oligonucleotides and chirally controlled DMD oligonucleotide compositions, wherein the DMD oligonucleotides (or DMD oligonucleotides of a plurality in chirally controlled DMD oligonucleotide compositions) optionally comprises one or more non-negatively charged internucleotidic linkages. Among other things, such DMD oligonucleotides and DMD oligonucleotide compositions can provide greatly improved effects, better delivery, lower toxicity, etc.

In some embodiments, a provided DMD oligonucleotide composition is administered at a dose and/or frequency lower than that of an otherwise comparable reference DMD oligonucleotide composition with comparable effect in altering the splicing of a target DMD transcript. In some embodiments, a stereocontrolled (chirally controlled) DMD oligonucleotide composition is administered at a dose and/or frequency lower than that of an otherwise comparable stereorandom reference DMD oligonucleotide composition with comparable effect in altering the splicing of the target DMD transcript. If desired, a provided composition can also be administered at higher dose/frequency due to its lower toxicities.

In some embodiments, provided DMD oligonucleotides, compositions and methods have low toxicities, e.g., when compared to a reference composition. As widely known in the art, DMD oligonucleotides can induce toxicities when administered to, e.g., cells, tissues, organism, etc. In some embodiments, DMD oligonucleotides can induce undesired immune response. In some embodiments, DMD oligonucleotide can induce complement activation. In some embodiments, DMD oligonucleotides can induce activation of the alternative pathway of complement. In some embodiments, DMD oligonucleotides can induce inflammation. Among other things, the complement system has strong cytolytic activity that can damages cells and should therefore be modulated to reduce potential injuries. In some embodiments, DMD oligonucleotide-induced vascular injury is a recurrent challenge in the development of DMD oligonucleotides for e.g., pharmaceutical use. In some embodiments, a primary source of inflammation when high doses of DMD oligonucleotides are administered involves activation of the alternative complement cascade. In some embodiments, complement activation is a common challenge associated with phosphorothioate-containing DMD oligonucleotides, and there is also a potential of some sequences of phosphorothioates to induce innate immune cell activation. In some embodiments, cytokine release is associated with administration of DMD oligonucleotides. For example, in some embodiments, increases in interleukin-6 (IL-6) monocyte chemoattractant protein (MCP-1) and/or interleukin-12 (IL-12) is observed. See, e.g., Frazier, Antisense Oligonucleotide Therapies: The Promise and the Challenges from a Toxicologic Pathologist's Perspective. Toxicol Pathol., 43: 78-89, 2015; and Engelhardt, et al., Scientific and Regulatory Policy Committee Points-to-consider Paper: Drug-induced Vascular Injury Associated with Nonsmall Molecule Therapeutics in Preclinical Development: Part 2. Antisense Oligonucleotides. Toxicol Pathol. 43: 935-944, 2015.

Oligonucleotide compositions as provided herein can be used as agents for modulating a number of cellular processes and machineries, including but not limited to, DMD transcription, translation, immune responses, epigenetics, etc. In addition, DMD oligonucleotide compositions as provided herein can be used as reagents for research and/or diagnostic purposes. One of ordinary skill in the art will readily recognize that the present disclosure herein is not limited to particular use but is applicable to any situations where the use of synthetic oligonucleotides is desirable. Among other things, provided compositions are useful in a variety of therapeutic, diagnostic, agricultural, and/or research applications.

Various dosing regimens can be utilized to administer provided chirally controlled DMD oligonucleotide compositions, e.g., those described in in U.S. Pat. Nos. 9,695,211, 9,605,019, 9,598,458, US 2013/0178612, US 20150211006, US 20170037399, WO 2017/015555, WO 2017/062862, WO 2017/160741, WO 2017/192664. WO 2017/192679, and/or WO 2017/210647, the dosing regimens of each of which is incorporated herein by reference.

In some embodiments, with their low toxicity, provided DMD oligonucleotides and compositions can be administered in higher dosage and/or with higher frequency. In some embodiments, with their improved delivery (and other properties), provided compositions can be administered in lower dosages and/or with lower frequency to achieve biological effects, for example, clinical efficacy.

A single dose can contain various amounts of DMD oligonucleotides. In some embodiments, a single dose can contain various amounts of a type of chirally controlled DMD oligonucleotide, as desired suitable by the application. In some embodiments, a single dose contains about 1, 5, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300 or more (e.g., about 350, 400, 450, 500, 550, 600, 650, 700, 750, 800, 850, 900, 950, 1000 or more) mg of a type of chirally controlled DMD oligonucleotide. In some embodiments, a chirally controlled DMD oligonucleotide is administered at a lower amount in a single dose, and/or in total dose, than a chirally uncontrolled DMD oligonucleotide. In some embodiments, a chirally controlled DMD oligonucleotide is administered at a lower amount in a single dose, and/or in total dose, than a chirally uncontrolled DMD oligonucleotide due to improved efficacy. In some embodiments, a chirally controlled DMD oligonucleotide is administered at a higher amount in a single dose, and/or in total dose, than a chirally uncontrolled DMD oligonucleotide. In some embodiments, a chirally controlled DMD oligonucleotide is administered at a higher amount in a single dose, and/or in total dose, than a chirally uncontrolled DMD oligonucleotide due to improved safety.

### Pharmaceutical Compositions

When used as therapeutics, a provided DMD oligonucleotide or DMD oligonucleotide composition described herein is administered as a pharmaceutical composition. In some embodiments, the pharmaceutical composition comprises a therapeutically effective amount of a provided DMD oligonucleotides, or a pharmaceutically acceptable salt thereof, and at least one pharmaceutically acceptable inactive ingredient selected from pharmaceutically acceptable diluents, pharmaceutically acceptable excipients, and pharmaceutically acceptable carriers. In some embodiments, in provided compositions provided DMD oligonucleotides may exist as salts, preferably pharmaceutically acceptable salts, e.g., sodium salts, ammonium salts, etc. In some embodiments, a salt of a provided DMD oligonucleotide comprises two or more cations, for example, in some embodiments, up to the number of negatively charged acidic groups (e.g., phosphate, phosphorothioate, etc.) in a DMD oligonucleotide. As appreciated by those skilled in the art, DMD oligonucleotides described herein may be provided and/or utilized in a salt form, particularly a pharmaceutically acceptable salt form.

In some embodiments, the present disclosure provides salts of provided DMD oligonucleotides, e.g., chirally controlled DMD oligonucleotides, and pharmaceutical compositions thereof. In some embodiments, a salt is a pharmaceutically acceptable salt. In some embodiments, each hydrogen ion that may be donated to a base (e.g., under conditions of an aqueous solution, a pharmaceutical composition, etc.) is replaced by a non-H* cation. For example, in some embodiments, a pharmaceutically acceptable salt of a DMD oligonucleotide is an all-metal ion salt, wherein each hydrogen ion (for example, of —OH, —SH, etc., acidic enough in water) of each internucleotidic linkage (e.g., a natural phosphate linkage, a phosphorothioate diester linkage, etc.) is replaced by a metal ion. In some embodiments, a provided salt is an all-sodium salt. In some embodiments, a provided pharmaceutically acceptable salt is an all-sodium salt. In some embodiments, a provided salt is an all-sodium salt, wherein each internucleotidic linkage which is a natural phosphate linkage (acid form —O—P(O)(OH)—O—), if any, exists as its sodium salt form (—O—P(O)(ONa)—O—), and each internucleotidic linkage which is a phosphorothioate diester linkage (phosphorothioate internucleotidic linkage; acid form —O—P(O)(SH)—O—), if any, exists as its sodium salt form (—O—P(O)(SNa)—O—).

In some embodiments, the pharmaceutical composition is formulated for intravenous injection, oral administration, buccal administration, inhalation, nasal administration, topical administration, ophthalmic administration or otic administration. In some embodiments, the pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

In some embodiments, the present disclosure provides a pharmaceutical composition comprising chirally controlled DMD oligonucleotide, or composition thereof, in admixture with a pharmaceutically acceptable excipient. One of skill in the art will recognize that the pharmaceutical compositions include the pharmaceutically acceptable salts of the chirally controlled DMD oligonucleotide, or composition thereof, described above.

A variety of supramolecular nanocarriers can be used to deliver nucleic acids. Example nanocarriers include, but are not limited to liposomes, cationic polymer complexes and various polymeric. Complexation of nucleic acids with various polycations is another approach for intracellular delivery; this includes use of PEGlyated polycations, polyethyleneamine (PEI) complexes, cationic block co-polymers, and dendrimers. Several cationic nanocarriers, including PEI and polyamidoamine dendrimers help to release contents from endosomes. Other approaches include use of polymeric nanoparticles, polymer micelles, quantum dots and lipoplexes. In some embodiments, a DMD oligonucleotide is conjugated to another molecular.

Additional nucleic acid delivery strategies are known in addition to the example delivery strategies described herein.

In therapeutic and/or diagnostic applications, the compounds of the disclosure can be formulated for a variety of modes of administration, including systemic and topical or localized administration. Techniques and formulations generally may be found in Remington, The Science and Practice of Pharmacy, (20th ed. 2000).

Provided DMD oligonucleotides, and compositions thereof, are effective over a wide dosage range. For example, in the treatment of adult humans, dosages from about 0.01 to about 1000 mg, from about 0.5 to about 100 mg, from about 1 to about 50 mg per day, and from about 5 to about 100 mg per day are examples of dosages that may be used. The exact dosage will depend upon the route of administration, the form in which the compound is administered, the subject to be treated, the body weight of the subject to be treated, and the preference and experience of the attending physician.

Pharmaceutically acceptable salts are generally well known to those of ordinary skill in the art, and may include, by way of example but not limitation, acetate, benzenesulfonate, besylate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington, The Science and Practice of Pharmacy (20th ed. 2000). Preferred pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

As appreciated by a person having ordinary skill in the art, DMD oligonucleotides may be formulated as a number of salts for, e.g., pharmaceutical uses. In some embodiments, a salt is a metal cation salt and/or ammonium salt. In some embodiments, a salt is a metal cation salt of a DMD oligonucleotide. In some embodiments, a salt is an ammonium salt of a DMD oligonucleotide. Representative alkali or alkaline earth metal salts include sodium, lithium, potassium, calcium, magnesium, and the like. In some embodiments, a salt is a sodium salt of a DMD oligonucleotide. In some embodiments, pharmaceutically acceptable salts include, when appropriate, nontoxic ammonium, quaternary ammonium, and amine cations formed with DMD oligonucleotides. As appreciated by a person having ordinary skill in the art, a salt of a DMD oligonucleotide may contain more than one cations, e.g., sodium ions, as there may be more than one anions within a DMD oligonucleotide.

Depending on the specific conditions being treated, such agents may be formulated into liquid or solid dosage forms and administered systemically or locally. The agents may be delivered, for example, in a timed- or sustained- low release form as is known to those skilled in the art. Techniques for formulation and administration may be found in Remington, The Science and Practice of Pharmacy (20th ed. 2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection.

Compounds, e.g., DMD oligonucleotides, can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure may also be formulated by methods known to those of skill in the art, and may include, for example, but not limited to, examples of solubilizing, diluting, or dispersing substances such as, saline, preservatives, such as benzyl alcohol, absorption promoters, and fluorocarbons.

In certain embodiments, DMD oligonucleotides and compositions are delivered to the CNS. In certain embodiments, DMD oligonucleotides and compositions are delivered to the cerebrospinal fluid. In certain embodiments, DMD oligonucleotides and compositions are administered to the brain parenchyma. In certain embodiments, DMD oligonucleotides and compositions are delivered to an animal/subject by intrathecal administration, or intracerebroventricular administration. Broad distribution of DMD oligonucleotides and compositions, described herein, within the central nervous system may be achieved with intraparenchymal administration, intrathecal administration, or intracerebroventricular administration.

In certain embodiments, parenteral administration is by injection, by, e.g., a syringe, a pump, etc. In certain embodiments, the injection is a bolus injection. In certain embodiments, the injection is administered directly to a tissue, such as striatum, caudate, cortex, hippocampus and cerebellum.

In certain embodiments, methods of specifically localizing a pharmaceutical agent, such as by bolus injection, decreases median effective concentration (EC50) by a factor of 20, 25, 30, 35, 40, 45 or 50. In certain embodiments, the targeted tissue is brain tissue. In certain embodiments the targeted tissue is striatal tissue. In certain embodiments, decreasing EC50 is desirable because it reduces the dose required to achieve a pharmacological result in a patient in need thereof.

In certain embodiments, a DMD oligonucleotide is delivered by injection or infusion once every month, every two months, every 90 days, every 3 months, every 6 months, twice a year or once a year.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of an active compound into preparations which can be used pharmaceutically. The preparations formulated for oral administration may be in the form of tablets, dragees, capsules, or solutions.

Pharmaceutical preparations for oral use can be obtained by combining an active compound with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, an active compound may be dissolved or suspended in suitable liquids, such as fatty oils, liquid paraffin, or liquid polyethylene glycols (PEGs). In addition, stabilizers may be added.

In some embodiments, any DMD oligonucleotide, or combination thereof, described herein, or any composition comprising a DMD oligonucleotide described herein, can be combined with any pharmaceutical preparation described herein or known in the art.

### Combination Therapy

In some embodiments, a subject is administered an additional treatment (including, but not limited to, a therapeutic agent or method) in additional to provided DMD oligonucleotide or DMD oligonucleotide composition, e.g., a composition comprising a DMD oligonucleotide. In some embodiments, a composition comprising a DMD oligonucleotide(s) (or two or more compositions, each comprising a DMD oligonucleotide) is administered to a patient along with an additional treatment.

In some embodiments, the present disclosure pertains to a method for treating muscular dystrophy, Duchenne (Duchenne's) muscular dystrophy (DMD), or Becker (Becker's) muscular dystrophy (BMD), comprising (a) administering to a subject susceptible thereto or suffering therefrom a composition comprising a provided DMD oligonucleotide, and (b) administering to the subject an additional treatment which is capable of preventing, treating, ameliorating or slowing the progress of muscular dystrophy. In some embodiments, an additional treatment is a composition comprising a second DMD oligonucleotide.

In some embodiments, an additional treatment is capable of preventing, treating, ameliorating or slowing the progress of muscular dystrophy by itself. In some embodiments, an additional treatment is capable of preventing, treating, ameliorating or slowing the progress of muscular dystrophy when administered with a provided DMD oligonucleotide.

In some embodiments, an additional treatment is administered to the subject prior to, after or simultaneously with a composition comprising a provided DMD oligonucleotide, e.g., a provided DMD oligonucleotide. In some embodiments, a composition comprises both a DMD oligonucleotide(s) and an additional treatment. In some embodiments, a DMD oligonucleotide(s) and an additional treatment(s) are in separate compositions. In some embodiments, the present disclosure provides technologies (e.g., compositions, methods, etc.) for combination therapy, for example, with other therapeutic agents and/or medical procedures. In some embodiments, provided DMD oligonucleotides and/or compositions may be used together with one or more other therapeutic agents. In some embodiments, provided compositions comprise provided DMD oligonucleotides, and one or more other therapeutic agents. In some embodiments, the one or more other therapeutic agents may have one or more different targets, and/or one or more different mechanisms toward targets, when compared to provided DMD oligonucleotides in the composition. In some embodiments, a therapeutic agent is a DMD oligonucleotide. In some embodiments, a therapeutic agent is a small molecule drug. In some embodiments, a therapeutic agent is a protein. In some embodiments, a therapeutic agent is an antibody. A number of therapeutic agents may be utilized in accordance with the present disclosure. For example, DMD oligonucleotides for DMD may be used together with one or more therapeutic agents that modulate utrophin production (utrophin modulators). In some embodiments, a utrophin modulator promotes production of utrophin. In some embodiments, a utrophin modulator is ezutromid. In some embodiments, a utrophin modulator is

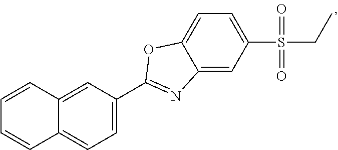 [SMILES: CCS(=O)(=O)c1ccc2oc(-c3ccc4ccccc4c3)nc2c1]

or a pharmaceutically acceptable salt thereof. In some embodiments, provided DMD oligonucleotides or compositions thereof are administered prior to, concurrently with, or subsequent to one or more other therapeutic agents and/or medical procedures. In some embodiments, provided DMD oligonucleotides or compositions thereof are administered concurrently with one or more other therapeutic agents and/or medical procedures. In some embodiments, provided DMD oligonucleotides or compositions thereof are administered prior to one or more other therapeutic agents and/or medical procedures. In some embodiments, provided DMD oligonucleotides or compositions thereof are administered subsequent to one or more other therapeutic agents and/or medical procedures. In some embodiments, provide compositions comprise one or more other therapeutic agents.

In some embodiments, a composition comprising a DMD oligonucleotide is co-administered with an additional agent in order to improve skipping of a DMD exon of interest. In some embodiments, an additional agent is an antibody, DMD oligonucleotide, protein or small molecule. In some embodiments, an additional agent interferes with a protein involved in splicing. In some embodiments, an additional agent interferes with a protein involved in splicing, wherein the protein is a SR protein.

In some embodiments, an additional agent interferes with a protein involved in splicing, wherein the protein is a SR protein, which contains a protein domain with one or more long repeats of serine (S) and arginine (R) amino acid residues. SR proteins are reportedly heavily phosphorylated in cells and are involved in constitutive and alternative splicing. Long et al. 2009 Biochem. J. 417: 15-27; Shepard et al. 2009 Genome Biol. 10: 242. In some embodiments, an additional agent is a chemical compound that inhibits or decreases a SR protein kinase. In some embodiments, a chemical compound that inhibits or decreases a SR protein kinase is SRPIN340. SRPIN340 is reported in, for example, Fukuhura et al. 2006 Proc. Natl. Acad. Sci. USA103: 11329-11333. In some embodiments, a chemical compound is a kinase inhibitor specific for Cdc-like kinases (Clks) that are also able to phosphorylate SR proteins. In some embodiments, a kinase inhibitor specific for Cdc-like kinases (Clks) that are also able to phosphorylate SR proteins is TG⁰⁰³. TG⁰⁰³ reportedly affected splicing both in vitro and in vivo. Nowak et al. 2010 J. Biol. Chem. 285: 5532-5540; Muraki et al. 2004 J. Biol. Chem. 279: 24246-24254; Yomoda et al. 2008 Genes Cells 13: 233-244; and Nishida et al. 2011 Nat Commun. 2:308.

In some embodiments, in a patient afflicted with muscular dystrophy, muscle tissue is replaced by fat and connective tissue, and affected muscles may look larger due to increased fat content, a condition known as pseudohypertrophy. In some embodiments, a composition comprising a DMD oligonucleotide(s) is administered along with a treatment which reduces or prevents development of fat or fibrous or connective tissue, or replacement of muscle tissue by fat or fibrous or connective tissue.

In some embodiments, a composition comprising a DMD oligonucleotide(s) is administered along with a treatment which reduces or prevents development of fat or fibrous or connective tissue, or replacement of muscle tissue by fat or fibrous or connective tissue, wherein the treatment is an antibody to connective tissue growth factor (CTGF), a central mediator of fibrosis (e.g., FG-3019). In some embodiments, a composition comprising a DMD oligonucleotide(s) is administered along with an agent which reduces the fat content of the human body.

Additional treatments include: slowing the progression of the disease by immune modulators (eg, steroids and transforming growth factor-beta inhibitors), inducing or introducing proteins that may compensate for dystrophin deficiency in the myofiber (eg, utrophin, biglycan, and laminin), or bolstering the muscle's regenerative response (eg, myostatin and activin 2B).

In some embodiments, an additional treatment is a small molecule capable of restoring normal balance of calcium within muscle cells.

In some embodiments, an additional treatment is a small molecule capable of restoring normal balance of calcium within muscle cells by correcting the activity of a type of channel called the ryanodine receptor calcium channel complex (RyR). In some embodiments, such a small molecule is Rycal ARM210 (ARMGO Pharma, Tarry Town, NY).

In some embodiments, an additional treatment is a flavonoid.

In some embodiments, an additional treatment is a flavonoid such as Epicatechin. Epicatechin is a flavonoid found in dark chocolate harvested from the cacao tree which has been reported in animals and humans to increase the production of new mitochondria in heart and muscle (e.g., mitochondrial biogenesis) while concurrently stimulating the regeneration of muscle tissue.

In some embodiments, an additional treatment is follistatin gene therapy.

In some embodiments, an additional treatment is adeno-associated virus delivery of follistatin 344 to increase muscle strength and prevent muscle wasting and fibrosis.

In some embodiments, an additional treatment is glucocorticoid.

In some embodiments, an additional treatment is prednisone.

In some embodiments, an additional treatment is deflazacort.

In some embodiments, an additional treatment is vamorolone (VBP15).

In some embodiments, an additional treatment is delivery of an exogenous Dystrophin gene or synthetic version or portion thereof, such as a microdystrophin gene.

In some embodiments, an additional treatment is delivery of an exogenous Dystrophin gene or portion thereof, such as a microdystrophin gene, such as SGT-001, an adeno-associated viral (AAV) vector-mediated gene transfer system for delivery of a synthetic dystrophin gene or microdystrophin (Solid BioSciences, Cambridge, Mass.).

In some embodiments, an additional treatment is stem cell treatment.

In some embodiments, an additional treatment is a steroid.

In some embodiments, an additional treatment is a corticosteroid.

In some embodiments, an additional treatment is prednisone.

In some embodiments, an additional treatment is a beta-2 agonist.

In some embodiments, an additional treatment is an ion channel inhibitor.

In some embodiments, an additional treatment is a calcium channel inhibitor.

In some embodiments, an additional treatment is a calcium channel inhibitor which is a xanthin. In some embodiments, an additional treatment is a calcium channel inhibitor which is methylxanthine. In some embodiments, an additional treatment is a calcium channel inhibitor which is pentoxifylline. In some embodiments, an additional treatment is a calcium channel inhibitor which is a methylxanthine derivative selected from: pentoxifylline, furafylline, lisofylline, propentofylline, pentifylline, theophylline, torbafylline, albifylline, enprofylline and derivatives thereof.

In some embodiments, an additional treatment is a treatment for heart disease or cardiovascular disease.

In some embodiments, an additional treatment is a blood pressure medicine.

In some embodiments, an additional treatment is surgery.

In some embodiments, an additional treatment is surgery to fix shortened muscles, straighten the spine, or treat a heart or lung problem.

In some embodiments, an additional treatment is a brace, walker, standing walker, or other mechanical aid for walking.

In some embodiments, an additional treatment is exercise and/or physical therapy.

In some embodiments, an additional treatment is assisted ventilation.

In some embodiments, an additional treatment is anticonvulsant, immunosuppressant or treatment for constipation.

In some embodiments, an additional treatment is an inhibitor of NF-κB.

In some embodiments, an additional treatment comprises salicylic acid and/or docosahexaenoic acid (DHA).

In some embodiments, an additional treatment is edasalonexent (CAT-1004, Catabasis), a conjugate of salicylic acid and docosahexaenoic acid (DHA).

In some embodiments, an additional treatment is a cell-based therapeutic.

In some embodiments, an additional treatment is comprises allogeneic cardiosphere-derived cells.

In some embodiments, an additional treatment is CAP-1002 (Capricor).

In some embodiments, y, t, n and m, e.g., in a stereochemistry pattern, each are independently 1-20 as described in the present disclosure. In some embodiments, y is 1. In some embodiments, y is at least 2, 3, 4, 5, 6, 7, 8, 9, 10, 11, 12, 13, 14, or 15. In some embodiments, y is 2, 3, 4, 5, 6, 7, 8, 9, 10, 11, 12, 13, 14, or 15. In some embodiments, y is 1, 2, 3, 4, 5, 6, 7, 8, 9, or 10. In some embodiments, y is 1. In some embodiments, y is 2. In some embodiments, y is 3. In some embodiments, y is 4. In some embodiments, y is 5. In some embodiments, y is 6. In some embodiments, y is 7. In some embodiments, y is 8. In some embodiments, y is 9. In some embodiments, y is 10.

In some embodiments, n is 1. In some embodiments, n is at least 2, 3, 4, 5, 6, 7, 8, 9, 10, 11, 12, 13, 14, or 15. In some embodiments, n is 2, 3, 4, 5, 6, 7, 8, 9, 10, 11, 12, 13, 14, or 15. In some embodiments, n is 1-10. In some embodiments, n is 1, 2, 3, 4, 5, 6, 7 or 8. In some embodiments, n is 1. In some embodiments, n is 2, 3, 4, 5, 6, 7 or 8. In some embodiments, n is 3, 4, 5, 6, 7 or 8. In some embodiments, n is 4, 5, 6, 7 or 8. In some embodiments, n is 5, 6, 7 or 8. In some embodiments, n is 6, 7 or 8. In some embodiments, n is 7 or 8. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, m is 0-50. In some embodiments, m is 1-50. In some embodiments, m is 1. In some embodiments, m is 2-50. In some embodiments, m is at least 2, 3, 4, 5, 6, 7, 8, 9, 10, 11, 12, 13, 14, or 15. In some embodiments, m is 2, 3, 4, 5, 6, 7 or 8. In some embodiments, m is 3, 4, 5, 6, 7 or 8. In some embodiments, m is 4, 5, 6, 7 or 8. In some embodiments, m is 5, 6, 7 or 8. In some embodiments, m is 6, 7 or 8. In some embodiments, m is 7 or 8. In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12. In some embodiments, m is 13. In some embodiments, m is 14. In some embodiments, m is 15. In some embodiments, m is 16. In some embodiments, m is 17. In some embodiments, m is 18. In some embodiments, m is 19. In some embodiments, m is 20. In some embodiments, m is 21. In some embodiments, m is 22. In some embodiments, in is 23. In some embodiments, m is 24. In some embodiments, m is 25. In some embodiments, m is at least 2. In some embodiments, m is at least 3. In some embodiments, m is at least 4. In some embodiments, m is at least 5. In some embodiments, m is at least 6. In some embodiments, m is at least 7. In some embodiments, m is at least 8. In some embodiments, m is at least 9. In some embodiments, m is at least 10. In some embodiments, in is at least 11. In some embodiments, m is at least 12. In some embodiments, m is at least 13. In some embodiments, m is at least 14. In some embodiments, m is at least 15. In some embodiments, m is at least 16. In some embodiments, m is at least 17. In some embodiments, m is at least 18. In some embodiments, m is at least 19. In some embodiments, m is at least 20. In some embodiments, m is at least 21. In some embodiments, m is at least 22. In some embodiments, m is at least 23. In some embodiments, m is at least 24. In some embodiments, m is at least 25. In some embodiments, m is at least greater than 25.

In some embodiments, t is 1-20. In some embodiments, t is 1. In some embodiments, t is at least 2, 3, 4, 5, 6, 7, 8, 9, 10, 11, 12, 13, 14, or 15. In some embodiments, t is 2, 3, 4, 5, 6, 7, 8, 9, 10, 11, 12, 13, 14, or 15. In some embodiments, t is 1-5. In some embodiments, t is 2. In some embodiments, t is 3. In some embodiments, t is 4. In some embodiments, t is 5. In some embodiments, t is 6. In some embodiments, t is 7. In some embodiments, t is 8. In some embodiments, t is 9. In some embodiments, t is 10. In some embodiments, t is 11. In some embodiments, t is 12. In some embodiments, t is 13. In some embodiments, t is 14. In some embodiments, t is 15. In some embodiments, t is 16. In some embodiments, t is 17. In some embodiments, t is 18. In some embodiments, t is 19. In some embodiments, t is 20.

In some embodiments, each of t and m is independently at least 2, 3, 4, 5, 6, 7, 8, 9, 10, 11, 12, 13, 14, or 15. In some embodiments, each of t and m is independently at least 3. In some embodiments, each oft and m is independently at least 4. In some embodiments, each oft and m is independently at least 5. In some embodiments, each oft and m is independently at least 6. In some embodiments, each oft and m is independently at least 7. In some embodiments, each of t and m is independently at least 8. In some embodiments, each of t and m is independently at least 9. In some embodiments, each of t and m is independently at least 10.

As used in the present disclosure, in some embodiments, “one or more” is 1-200, 1-150, 1-100, 1-90, 1-80, 1-70, 1-60, 1-50, 1-40, 1-30, or 1, 2, 3, 4, 5, 6, 7, 8, 9, 10, 11, 12, 13, 14, 15, 16, 17, 18, 19, 20, 21, 22, 23, 24, or 25. In some embodiments, “one or more” is one. In some embodiments, “one or more” is two. In some embodiments, “one or more” is three. In some embodiments, “one or more” is four. In some embodiments, “one or more” is five. In some embodiments, “one or more” is six. In some embodiments, “one or more” is seven. In some embodiments, “one or more” is eight. In some embodiments, “one or more” is nine. In some embodiments, “one or more” is ten. In some embodiments, “one or more” is at least one. In some embodiments, “one or more” is at least two. In some embodiments, “one or more” is at least three. In some embodiments, “one or more” is at least four. In some embodiments, “one or more” is at least five. In some embodiments, “one or more” is at least six. In some embodiments, “one or more” is at least seven. In some embodiments, “one or more” is at least eight. In some embodiments, “one or more” is at least nine. In some embodiments, “one or more” is at least ten. As used in the present disclosure, in some embodiments, “at least one” is 1-200, 1-150, 1-100, 1-90, 1-80, 1-70, 1-60, 1-50, 1-40, 1-30, or 1, 2, 3, 4, 5, 6, 7, 8, 9, 10, 11, 12, 13, 14, 15, 16, 17, 18, 19, 20, 21, 22, 23, 24, or 25. In some embodiments, “at least one” is one. In some embodiments, “at least one” is two. In some embodiments, “at least one” is three. In some embodiments, “at least one” is four. In some embodiments, “at least one” is five. In some embodiments, “at least one” is six. In some embodiments, “at least one” is seven. In some embodiments, “at least one” is eight. In some embodiments, “at least one” is nine. In some embodiments, “at least one” is ten.

Among other things, the present disclosure provides the following Example Embodiments:

1. An oligonucleotide having the structure of:

(SEQ ID NO: 172)
fG*SfU*SfAn001RfC*SfC*SfUn001RfC*SfC*SmAfA*SmC*
SfA*SmUfC*SfA*SfA*SfGn001RfG*SfA*SfA;
(SEQ ID NO: 175)
fU*SfG*SfGn001RfC*SfA*SfUn001RfU*SfU*SmCfU*SmA*
SfG*SmUfU*SfU*SfG*SfGn001RfA*SfG*SfA;
(SEQ ID NO: 188)
fG*SfG*SfCn001RfA*SfU*SfUn001RmUfC*SfU*SmA*SfG*
SmUmU*SfU*SfG*SfG*SfAn001RfG*SfA*SfU;
(SEQ ID NO: 178)
fU*SfG*SfGn001RfC*SfA*SfGn001RfU*SfU*SmUfC*SmC*
SfU*SmUfA*SfG*SfU*SfAn001RfA*SfC*SfC;
(SEQ ID NO: 190)
fU*SfG*SfGn001RfC*SfA*SfGn001RmUfU*SfU*SmC*SfC*
SmUmU*SfA*SfG*SfU*SfAn001RfA*SfC*SfC;
(SEQ ID NO: 171)
fG*SfG*SfUn001RfA*SfA*SfGn001RfU*SfU*SmCfU*SmG*
SfU*SmCfC*SfA*SfA*SfGn001RfC*SfC*SfC;
(SEQ ID NO: 183)
fG*SfG*SfUn001RfA*SfA*SfGn001RmUfU*SfC*SmU*SfG*
SmUmC*SfC*SfA*SfA*SfGn001RfC*SfC*SfC;
(SEQ ID NO: 173)
fC*SfA*SfAn001RfC*SfA*SfUn001RfC*SfA*SmAfG*SmG*
SfA*SmAfG*SfA*SfU*SfGn001RfG*SfC*SfA;
(SEQ ID NO: 174)
fA*SfU*SfGn001RfG*SfC*SfAn001RfU*SfU*SmUfC*SmU*
SfA*SmGfU*SfU*SfU*SfGn001RfG*SfA*SfG;
(SEQ ID NO: 186)
fA*SfU*SfGn001RfG*SfC*SfAn001RmUfU*SfU*SmC*SfU*
SmAmG*SfU*SfU*SfU*SfGn001RfG*SfA*SIG;
(SEQ ID NO: 177)
fG*SfC*SfAn001RfU*SfU*SfUn001RfC*SfU*SmAfG*SmU*
SfU*SmUfG*SfG*SfA*SfGn001RfA*SfU*SfG;
(SEQ ID NO: 179)
fC*SfA*SfGn001RfU*SfU*SfUn001RfC*SfC*SmUfU*SmA*
SfG*SmUfA*SfA*SfC*SfCn001RfA*SfC*SfA;
(SEQ ID NO: 192)
fA*SfG*SfUn001RfU*SfU*SfCn001RmCfU*SfU*SmA*SfG*
SmUmA*SfA*SfC*SfC*SfAn001RfC*SfA*SfG;
(SEQ ID NO: 193)
fU*SfU*SfCn001RfC*SfU*SfUn001RmAfG*SfU*SmA*SfA*
SmCmC*SfA*SfC*SfA*SfGn001RfG*SfU*SfU;

or a pharmaceutically acceptable salt form thereof, wherein:
f represents a 2′-F modified nucleoside;
*S represents a Sp phosphorothioate;
m represents a 2′-OMe modified nucleoside; and

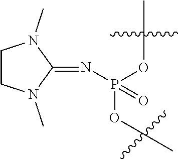 [SMILES: *OP(=O)(N=C1N(C)CCN1C)O*]

n001R is wherein the phosphorus is of the Rp configuration.

2. The oligonucleotide of Embodiments 1, wherein the oligonucleotide is in a salt form.

3. The oligonucleotide of Embodiment 2, wherein the salt form is a sodium salt.

4. The oligonucleotide of Embodiment 3, wherein the number of sodium ions in the sodium salt equals the total number of phosphorothioate and phosphate linkages in the oligonucleotide.

5. A chirally controlled oligonucleotide composition comprising a plurality of the oligonucleotide of any one of Embodiments 1-4, wherein it is enriched, relative to a substantially racemic preparation of oligonucleotides of the same base sequence of the oligonucleotide for the oligonucleotide.

6. A pharmaceutical composition, comprising a therapeutically effective amount of the oligonucleotide of any one of Embodiments 1-4 and a pharmaceutically acceptable inactive ingredient selected from pharmaceutically acceptable diluents, pharmaceutically acceptable excipients, and pharmaceutically acceptable carriers.

7. The pharmaceutical composition of Embodiment 10, wherein the pharmaceutical composition is a solution.

8. An oligonucleotide composition for use in treatment of a disease, said use comprising altering splicing of a target transcript,
wherein: the oligonucleotide composition being characterized in that, when it is contacted with the target transcript in a transcript splicing system, splicing of the transcript is altered relative to that observed under reference conditions selected from the group consisting of absence of the composition, presence of a reference composition, and combinations thereof.

9. The oligonucleotide composition for use of Embodiment 8, wherein
(a) the splicing of the target transcript is altered relative to absence of the composition, preferably wherein the target transcript is pre-mRNA of dystrophin, and wherein the alteration is that one or more exon is skipped at an increased level relative to absence of the composition, more preferably wherein exon 51 of dystrophin is skipped at an increased level relative to absence of the composition; or
(b) wherein the oligonucleotide composition is a composition of any one of Embodiments 6-7.

10. An oligonucleotide of any one of Embodiments 1 to 4, or a composition of any one of Embodiments 6-7 for use in treating Duchenne muscular dystrophy, said use comprising administering to a subject susceptible thereto or suffering therefrom an oligonucleotide of any one of Embodiments 1 to 9, or a composition of any one of Embodiments 9-13.

11. A method for preventing or treating DMD, comprising administering to a subject susceptible thereto or suffering therefrom an effective amount of a DMD oligonucleotide.

12. The method of Embodiment 15, wherein the subject has a mutation of the DMD gene that is amenable to exon 51 skipping, and the DMD oligonucleotide can provide exon 51 skipping.

13. The method of Embodiment 11, wherein the subject has a frameshift mutation of the DMD gene that is amenable to exon 51 skipping, and the DMD oligonucleotide can provide exon 51 skipping.

14. The method of Embodiment 11, wherein the oligonucleotide is an oligonucleotide of any one of Embodiments 1-4.

15. The method of Embodiment 15, wherein the oligonucleotide is administered in a composition of any one of Embodiments 5-7.

16. A method for preparing an oligonucleotide, comprising using of a chiral auxiliary, phosphoramidite or an azide reagent, or a condition described in the specification.

17. An oligonucleotide, chiral auxiliary, phosphoramidite, composition or method described in the specification.

Among other things, the present disclosure provides the following Example Embodiments:

1. An oligonucleotide composition, comprising a plurality of oligonucleotides of a particular oligonucleotide type defined by:
1) base sequence;
2) pattern of backbone linkages;
3) pattern of backbone chiral centers; and
4) pattern of backbone phosphorus modifications,
   wherein:
oligonucleotides of the plurality comprise at least 1, 2, 3, 4, 5, 6, 7, 8, 9, 10, 11, 12, 13, 14, 15, 16, 17, 18, 19, or 20 chirally controlled internucleotidic linkages; and
the oligonucleotide composition being characterized in that, when it is contacted with a transcript in a transcript splicing system, splicing of the transcript is altered relative to that observed under a reference condition selected from the group consisting of absence of the composition, presence of a reference composition, and combinations thereof.

2. The composition of any one of the preceding embodiments, wherein the transcript is a Dystrophin transcript.

3. The composition of any one of the preceding embodiments, wherein splicing of the transcript is altered such that the level of skipping of exon 45, 51, or 53, or multiple exons is increased.

4. The composition of any one of the preceding embodiments, wherein each chiral internucleotidic linkage of the oligonucleotides of the plurality is independently a chirally controlled internucleotidic linkage.

5. The composition of any one of the preceding embodiments, wherein each chiral modified internucleotidic linkage independently has a stereopurity of at least 90%, 91%, 92%, 93%, 94%, 95%, 96%, 97%, 98%, or 99% at its chiral linkage phosphorus.

6. The composition of any one of the preceding embodiments, wherein the base sequence is or comprises or comprises 15 contiguous bases of the base sequence of any oligonucleotide in Table A1.

7. The composition of any one of the preceding embodiments, wherein the pattern of backbone linkages comprises at least one non-negatively charged internucleotidic linkage.

8. The composition of any one of the preceding embodiments, wherein the pattern of backbone linkages comprises at least one non-negatively charged internucleotidic linkage which is a neutral internucleotidic linkage.

9. The composition of any one of the preceding embodiments, wherein the pattern of backbone linkages comprises at least one neutral internucleotidic linkage which is or comprises a triazole, neutral triazole, alkyne, or a cyclic guanidine.

10. The composition of any one of the preceding embodiments, wherein the oligonucleotide type comprises any of: cholesterol; L-carnitine (amide and carbamate bond); Folic acid; Gambogic acid; Cleavable lipid (1,2-dilaurin and ester bond); Insulin receptor ligand; CPP; Glucose (tri- and hex-antennary): or Mannose (tri- and hex-antennary, alpha and beta).

11. The composition of any one of the preceding embodiments, wherein the oligonucleotide type is any oligonucleotide listed in Table A1.

12. A composition comprising a plurality of oligonucleotides of a particular oligonucleotide type defined by:
1) base sequence;
2) pattern of backbone linkages;
3) pattern of backbone chiral centers; and
4) pattern of backbone phosphorus modifications,
which composition is chirally controlled and it is enriched, relative to a substantially racemic preparation of oligonucleotides having the same base sequence, pattern of backbone linkages and pattern of backbone phosphorus modifications, for oligonucleotides of the particular oligonucleotide type, wherein:
the oligonucleotide composition is characterized in that, when it is contacted with a transcript in a transcript splicing system, splicing of the transcript is altered in that level of skipping of an exon is increased relative to that observed under a reference condition selected from the group consisting of absence of the composition, presence of a reference composition, and combinations thereof.

13. The composition of any one of the preceding embodiments, wherein the transcript is a Dystrophin transcript.

14. The composition of any one of the preceding embodiments, wherein the exon is DMD exon 45, 51 or 53 or multiple DMD exons, and wherein the splicing of the transcript is altered such that the level of skipping of exon 45, 51, or 53, or multiple exons is increased.

15. The composition of any one of the preceding embodiments, wherein the pattern of backbone chiral centers comprises at least one Sp.

16. The composition of any one of the preceding embodiments, wherein the pattern of backbone chiral centers comprises at least one Rp.

17. The composition of any one of the preceding embodiments, wherein the composition is a chirally pure composition.

18. The composition of any one of the preceding embodiments, wherein each chiral modified internucleotidic linkage independently has a stereopurity of at least 90%, 91%, 92%, 93%, 94%, 95/, 96%, 97%, 98%, or 99% at its chiral linkage phosphorus.

19. The composition of any one of the preceding embodiments, wherein the base sequence is or comprises or comprises 15 contiguous bases of the base sequence of any oligonucleotide in Table A1.

20. The composition of any one of the preceding embodiments, wherein the pattern of backbone linkages comprises at least one non-negatively charged internucleotidic linkage.

21. The composition of any one of the preceding embodiments, wherein the pattern of backbone linkages comprises at least one non-negatively charged internucleotidic linkage which is a neutral internucleotidic linkage.

22. The composition of any one of the preceding embodiments, wherein the pattern of backbone linkages comprises at least one neutral internucleotidic linkage which is or comprises a triazole, neutral triazole, alkyne, or a cyclic guanidine.

23. The composition of any one of the preceding embodiments, wherein the oligonucleotide type comprises any of: cholesterol: L-carnitine (amide and carbamate bond); Folic acid: Gambogic acid; Cleavable lipid (1,2-dilaurin and ester bond); Insulin receptor ligand; CPP; Glucose (tri- and hex-antennary); or Mannose (tri- and hex-antennary, alpha and beta).

24. The composition of any one of the preceding embodiments, wherein the oligonucleotide type is any oligonucleotide listed in Table A1.

25. A composition comprising a plurality of oligonucleotides of a particular oligonucleotide type defined by:
1) base sequence;
2) pattern of backbone linkages; and
3) pattern of backbone phosphorus modifications, wherein:
oligonucleotides of the plurality comprise at least 1, 2, 3, 4, 5, 6, 7, 8, 9, 10, 11, 12, 13, 14, 15, 16, 17, 18, 19, or 20 non-negatively charged internucleotidic linkages;
the oligonucleotide composition is characterized in that, when it is contacted with a transcript in a transcript splicing system, splicing of the transcript is altered in that level of skipping of an exon is increased relative to that observed under a reference condition selected from the group consisting of absence of the composition, presence of a reference composition, and combinations thereof.

26. The composition of any one of the preceding embodiments, wherein the transcript is a Dystrophin transcript.

27. The composition of any one of the preceding embodiments, wherein the exon is DMD exon 45, 51, or 53 or multiple DMD exons, and the splicing of the transcript is altered such that the level of skipping of exon 45, 51, or 53, or multiple exons is increased.

28. The composition of any one of the preceding embodiments, wherein each non-negatively charged internucleotidic linkage is independently an internucleotidic linkage at least 50% of which exists in its non-negatively charged form at pH 7.4.

29. The composition of any one of the preceding embodiments, wherein each non-negatively charged internucleotidic linkage is independently a neutral internucleotidic linkage, wherein at least 50% of the internucleotidic linkage exists in its neutral form at pH 7.4.

30. The composition of any one of the preceding embodiments, wherein the neutral form of each non-negatively charged internucleotidic linkage independently has a pKa no less than 8, 9, 10, 11, 12, 13, or 14.

31. The composition of any one of the preceding embodiments, wherein the neutral form of each non-negatively charged internucleotidic linkage, when the units which it connects are replaced with —CH₃, independently has a pKa no less than 8, 9, 10, 11, 12, 13, or 14.

32. The composition of any one of the preceding embodiments, wherein the reference condition is absence of the composition.

33. The composition of any one of the preceding embodiments, wherein the reference condition is presence of a reference composition.

34. The composition of any one of the preceding embodiments, wherein the reference composition is an otherwise identical composition wherein the oligonucleotides of the plurality comprise no chirally controlled internucleotidic linkages.

35. The composition of any one of the preceding embodiments, wherein the reference composition is an otherwise identical composition wherein the oligonucleotides of the plurality comprise no non-negatively charged internucleotidic linkages.

36. The composition of any one of the preceding embodiments, wherein the pattern of backbone linkages comprises one or more backbone linkages selected from phosphodiester, phosphorothioate and phosphodithioate linkages.

37. The composition of any one of the preceding embodiments, wherein the oligonucleotides of the plurality each comprise one or more sugar modifications.

38. The composition of any one of the preceding embodiments, wherein the sugar modifications comprise one or more modifications selected from: 2′-O-methyl, 2′-MOE, 2′-F, morpholino and bicyclic sugar moieties.

39. The composition of any one of the preceding embodiments, wherein one or more sugar modifications are 2′-F modifications.

40. The composition of any one of the preceding embodiments, wherein the oligonucleotides of the plurality each comprise a 5′-end region comprising 1, 2, 3, 4, 5, 6, 7, 8, 9, 10 or more nucleoside units comprising a 2′-F modified sugar moiety.

41. The composition of any one of the preceding embodiments, wherein the oligonucleotides of the plurality each comprise a 3′-end region comprising 1, 2, 3, 4, 5, 6, 7, 8, 9, 10 or more nucleoside units comprising a 2′-F modified sugar moiety.

42. The composition of any one of the preceding embodiments, wherein the oligonucleotides of the plurality each comprise a middle region between the 5′-end region and the 3′-region comprising 1, 2, 3, 4, 5, 6, 7, 8, 9, 10 or more nucleotidic units comprising a phosphodiester linkage.

43. The composition of any one of the preceding embodiments, wherein the base sequence is or comprises or comprises 15 contiguous bases of the base sequence of any oligonucleotide in Table A1.

44. The composition of any one of the preceding embodiments, wherein the pattern of backbone linkages comprises at least one non-negatively charged internucleotidic linkage.

45. The composition of any one of the preceding embodiments, wherein the pattern of backbone linkages comprises at least one non-negatively charged internucleotidic linkage which is a neutral internucleotidic linkage.

46. The composition of any one of the preceding embodiments, wherein the pattern of backbone linkages comprises at least one neutral internucleotidic linkage which is or comprises a triazole, neutral triazole, alkyne, or a cyclic guanidine.

47. The composition of any one of the preceding embodiments, wherein the oligonucleotide type comprises any of: cholesterol; L-carnitine (amide and carbamate bond); Folic acid; Gambogic acid; Cleavable lipid (1,2-dilaurin and ester bond); Insulin receptor ligand; CPP; Glucose (tri- and hex-antennary); or Mannose (tri- and hex-antennary, alpha and beta).

48. The composition of any one of the preceding embodiments, wherein the oligonucleotide type is any oligonucleotide listed in Table A1.

49. A composition comprising a plurality of oligonucleotides of a particular oligonucleotide type defined by:
1) base sequence;
2) pattern of backbone linkages; and
3) pattern of backbone phosphorus modifications,
   wherein:
oligonucleotides of the plurality comprise:
1) a 5′-end region comprising 1, 2, 3, 4, 5, 6, 7, 8, 9, 10 or more nucleoside units comprising a 2′-F modified sugar moiety:
2) a 3′-end region comprising 1, 2, 3, 4, 5, 6, 7, 8, 9, 10 or more nucleoside units comprising a 2′-F modified sugar moiety; and
3) a middle region between the 5′-end region and the 3′-region comprising 1, 2, 3, 4, 5, 6, 7, 8, 9, 10 or more nucleotidic units comprising a phosphodiester linkage.

50. The composition of embodiment 43 or 49, wherein the oligonucleotide composition is characterized in that, when it is contacted with a transcript in a transcript splicing system, splicing of the transcript is altered in that level of skipping of an exon is increased relative to that observed under a reference condition selected from the group consisting of absence of the composition, presence of a reference composition, and combinations thereof.

51. The composition of any one of the preceding embodiments, wherein the transcript is a Dystrophin transcript.

52. The composition of any one of the preceding embodiments, wherein the exon is DMD exon 45, 51, or 53 or multiple DMD exons, and the splicing of the transcript is altered such that the level of skipping of exon 45, 51, or 53, or multiple exons is increased.

53. The composition of any one of the preceding embodiments, wherein the 5′-end region comprises 1 or more nucleoside units not comprising a 2′-F modified sugar moiety.

54. The composition of any one of the preceding embodiments, wherein the 3′-end region comprises 1 or more nucleoside units not comprising a 2′-F modified sugar moiety.

55. The composition of any one of the preceding embodiments, wherein the middle region comprises 1 or more nucleotidic units comprising no phosphodiester linkage.

56. The composition of any one of the preceding embodiments, wherein the first of the 1, 2, 3, 4, 5, 6, 7, 8, 9, 10 or more nucleoside units comprising a 2′-F modified sugar moiety and a modified internucleotidic linkage of the 5′-end is the first, second, third, fourth or fifth nucleoside unit of the oligonucleotide from the 5′-end, and the last of the 1, 2, 3, 4, 5, 6, 7, 8, 9, 10 or more nucleoside units comprising a 2′-F modified sugar moiety and a modified internucleotidic linkage of the 3′-end is the last, second last, third last, fourth last, or fifth last nucleoside unit of the oligonucleotide.

57. The composition of any one of the preceding embodiments, wherein the 5′-end region comprising 2, 3, 4, 5, 6, 7, 8, 9, 10 or more consecutive nucleoside units comprising a 2′-F modified sugar moiety.

58. The composition of any one of the preceding embodiments, wherein the 5′-end region comprising 5, 6, 7, 8, 9, 10 or more consecutive nucleoside units comprising a 2′-F modified sugar moiety.

59. The composition of any one of the preceding embodiments, wherein the 3′-end region comprising 2, 3, 4, 5, 6, 7, 8, 9, 10 or more consecutive nucleoside units comprising a 2′-F modified sugar moiety.

60. The composition of any one of the preceding embodiments, wherein the 3′-end region comprising 5, 6, 7, 8, 9, 10 or more consecutive nucleoside units comprising a 2′-F modified sugar moiety.

61. The composition of any one of the preceding embodiments, wherein each internucleotidic linkage between two nucleoside units comprising a 2-F modified sugar moiety in the 5′-end region is independently a modified internucleotidic linkage.

62. The composition of any one of the preceding embodiments, wherein each internucleotidic linkage between two nucleoside units comprising a 2′-F modified sugar moiety in the 3′-end region is independently a modified internucleotidic linkage.

63. The composition of embodiment 61 or 62, wherein each modified internucleotidic linkage is independently a chiral internucleotidic linkage.

64. The composition of embodiment 61 or 62, wherein each modified internucleotidic linkage is independently a chirally controlled internucleotidic linkage.

65. The composition of embodiment 61 or 62, wherein each modified internucleotidic linkage is a phosphorothioate internucleotidic linkage.

66. The composition of embodiment 61 or 62, wherein each modified internucleotidic linkage is a chirally controlled phosphorothioate internucleotidic linkage.

67. The composition of embodiment 61 or 62, wherein each modified internucleotidic linkage is a Sp chirally controlled phosphorothioate internucleotidic linkage.

68. The composition of any one of the preceding embodiments, wherein the middle region comprises 1, 2, 3, 4, 5, 6, 7, 8, 9, 10 or more natural phosphate linkages.

69. The composition of any one of the preceding embodiments, wherein the middle region comprises 1, 2, 3, 4, 5, 6, 7, 8, 9, 10 or more natural phosphate linkages each independently between a nucleoside unit comprising a 2′-OR¹ modified sugar moiety and a nucleoside unit comprising a 2′-F modified sugar moiety, or between two nucleoside units each independently comprising a 2′-OR¹ modified sugar moiety, wherein R¹ is optionally substituted C₁₋₆ alkyl.

70. The composition of any one of the preceding embodiments, wherein the middle region comprises 1, 2, 3, 4, 5, 6, 7, 8, 9, 10 or more non-negatively charged internucleotidic linkages.

71. The composition of any one of the preceding embodiments, wherein the middle region comprises 1, 2, 3, 4, 5, 6, 7, 8, 9, 10 or more non-negatively charged internucleotidic linkages each independently between a nucleoside unit comprising a 2′-OR¹ modified sugar moiety and a nucleoside unit comprising a 2′-F modified sugar moiety, or between two nucleoside units each independently comprising a 2′-OR¹ modified sugar moiety, wherein R¹ is optionally substituted C₁₋₆ alkyl.

72. The composition of embodiment 69 or 71, wherein 2′-OR¹ is 2′-OCH₃.

73. The composition of embodiment 69 or 71, wherein 2′-OR¹ is 2′-OCH₂CH₂OCH₃.

74. The composition of any one of the preceding embodiments, wherein the 5′-end region comprises at least 2, 3, 4, 5, 6, 7, 8, 9, or 10 chiral modified internucleotidic linkages.

75. The composition of any one of the preceding embodiments, wherein the 5′-end region comprises at least 2, 3, 4, 5, 6, 7, 8, 9, or 10 consecutive chiral modified internucleotidic linkages.

76. The composition of any one of the preceding embodiments, wherein each internucleotidic linkage in the 5′-end region is a chiral modified internucleotidic linkage.

77. The composition of any one of the preceding embodiments, wherein the 3′-end region comprises at least 2, 3, 4, 5, 6, 7, 8, 9, or 10 chiral modified internucleotidic linkages.

78. The composition of any one of the preceding embodiments, wherein the 3′-end region comprises at least 2, 3, 4, 5, 6, 7, 8, 9, or 10 consecutive chiral modified internucleotidic linkages.

79. The composition of any one of the preceding embodiments, wherein each internucleotidic linkage in the 3′-end region is a chiral modified internucleotidic linkage.

80. The composition of any one of the preceding embodiments, wherein the middle region comprises at least 2, 3, 4, 5, 6, 7, 8, 9, or 10 chiral modified internucleotidic linkages.

81. The composition of any one of the preceding embodiments, wherein the middle region comprises at least 2, 3, 4, 5, 6, 7, 8, 9, or 10 consecutive chiral modified internucleotidic linkages.

82. The composition of any one of embodiments 74-81, wherein each chiral modified internucleotidic linkage is independently a chirally controlled internucleotidic linkage.

83. The composition of any one of embodiments 74-81, wherein each chiral modified internucleotidic linkage is independently a chirally controlled internucleotidic linkage wherein its chirally controlled linkage phosphorus has a Sp configuration.

84. The composition of any one of embodiments 74-83, wherein each chiral modified internucleotidic linkage is independently a chirally controlled phosphorothioate internucleotidic linkage.

85. The composition of any one of the preceding embodiments, wherein the middle region comprises at least 2, 3, 4, 5, 6, 7, 8, 9, or 10 non-negatively charged internucleotidic linkages.

86. The composition of any one of the preceding embodiments, wherein the middle region comprises at least 2, 3, 4, 5, 6, 7, 8, 9, or 10 neutral internucleotidic linkages.

87. The composition of any one of the preceding embodiments, wherein a neutral internucleotidic linkage is a chiral internucleotidic linkage.

88. The composition of any one of the preceding embodiments, wherein a neutral internucleotidic linkage is a chirally controlled internucleotidic linkage independently of Rp or Sp at its linkage phosphorus.

89. The composition of any one of the preceding embodiments, wherein the base sequence comprises a sequence having no more than 5 mismatches from a 20 base long portion of the dystrophin gene or its complement.

90. The composition of any one of the preceding embodiments, wherein the length of the base sequence of the oligonucleotides of the plurality is no more than 50 bases.

91. The composition of any one of the preceding embodiments, wherein the pattern of backbone chiral centers comprises at least 3, 4, 5, 6, 7, 8, 9, 10, 11, 12, 13, 14, 15, 16, 17, 18, 19, 20, 21, 22, 23, 24, or 25 chirally controlled centers independently of Rp or Sp.

92. The composition of any one of the preceding embodiments, wherein the pattern of backbone chiral centers comprises at least 5 chirally controlled centers independently of Rp or Sp.

93. The composition of any one of the preceding embodiments, wherein the pattern of backbone chiral centers comprises at least 6 chirally controlled centers independently of Rp or Sp.

94. The composition of any one of the preceding embodiments, wherein the pattern of backbone chiral centers comprises at least 10 chirally controlled centers independently of Rp or Sp.

95. The composition of any one of the preceding embodiments, wherein the oligonucleotides of the particular oligonucleotide type are capable of mediating skipping of one or more exons of the dystrophin gene.

96. The composition of any one of the preceding embodiments, wherein the oligonucleotides of the plurality are capable of mediating the skipping of exon 45, 51 or 53 of the dystrophin gene.

97. The composition of embodiment 96, wherein the oligonucleotides of the plurality are capable of mediating the skipping of exon 45 of the dystrophin gene.

98. The composition of embodiment 96, wherein the oligonucleotides of the plurality are capable of mediating the skipping of exon 51 of the dystrophin gene.

99. The composition of embodiment 96, wherein the oligonucleotides of the plurality are capable of mediating the skipping of exon 53 of the dystrophin gene.

100. The composition of embodiment 97, wherein the base sequence comprises a sequence having no more than 5 mismatches from the sequence of any oligonucleotide disclosed herein.

101. The composition of embodiment 97, wherein the base sequence comprises or is the sequence of any oligonucleotide disclosed herein.

102. The composition of embodiment 97, wherein the base sequence is that of any oligonucleotide disclosed herein.

103. The composition of embodiment 97, wherein the base sequence comprises a sequence having no more than 5 mismatches from the sequence of any oligonucleotide disclosed herein.

104. The composition of embodiment 97, wherein the base sequence comprises or is any oligonucleotide disclosed herein.

105. The composition of embodiment 97, wherein the base sequence is any oligonucleotide disclosed herein.

106. The composition of any of the preceding embodiments, wherein the oligonucleotides of the plurality are any oligonucleotide disclosed herein.

107. The composition of embodiment 18, wherein oligonucleotides of the particular oligonucleotide type are any oligonucleotide disclosed herein.

108. The composition of any one of the preceding embodiments, wherein the base sequence is or comprises or comprises 15 contiguous bases of the base sequence of any oligonucleotide in Table A1.

109. The composition of any one of the preceding embodiments, wherein the pattern of backbone linkages comprises at least one non-negatively charged internucleotidic linkage.

110. The composition of any one of the preceding embodiments, wherein the oligonucleotides comprise 1, 2, 3, 4, 5, 6, 7, 8, 9, 10 or more non-negatively charged internucleotidic linkages.

111. The composition of any one of the preceding embodiments, wherein the oligonucleotides comprise 1, 2, 3, 4, 5, 6, 7, 8, 9, 10 or more chirally controlled non-negatively charged internucleotidic linkages.

112. The composition of any one of the preceding embodiments, wherein the oligonucleotides comprise 2, 3, 4, 5, 6, 7, 8, 9, 10 or more consecutive non-negatively charged internucleotidic linkages.

113. The composition of any one of the preceding embodiments, wherein the oligonucleotides comprise 2, 3, 4, 5, 6, 7, 8, 9, 10 or more consecutive chirally controlled non-negatively charged internucleotidic linkages.

114. The composition of any one of the preceding embodiments, wherein the oligonucleotides comprise a wing-core-wing, core-wing, or wing-core structure.

115. The composition of any one of the preceding embodiments, wherein a wing comprises 1, 2, 3, 4, 5, 6, 7, 8, 9, 10 or more non-negatively charged internucleotidic linkages.

116. The composition of any one of the preceding embodiments, wherein the oligonucleotides comprise a wing-core-wing, core-wing, or wing-core structure, and wherein a wing comprises 1, 2, 3, 4, 5, 6, 7, 8, 9, 10 or more chirally controlled non-negatively charged internucleotidic linkages.

117. The composition of any one of the preceding embodiments, wherein the oligonucleotides comprise a wing-core-wing, core-wing, or wing-core structure, and wherein a wing comprises 2, 3, 4, 5, 6, 7, 8, 9, 10 or more consecutive non-negatively charged internucleotidic linkages.

118. The composition of any one of the preceding embodiments, wherein the oligonucleotides comprise a wing-core-wing, core-wing, or wing-core structure, and wherein a wing comprises 2, 3, 4, 5, 6, 7, 8, 9, 10 or more consecutive chirally controlled non-negatively charged internucleotidic linkages.

119. The composition of any one of the preceding embodiments, wherein the oligonucleotides comprise or consist of a wing-core-wing structure, and wherein only one wing comprise one or more non-negatively charged internucleotidic linkages.

120. The composition of any one of the preceding embodiments, wherein the oligonucleotides comprise a wing-core-wing, core-wing, or wing-core structure, and wherein a core comprises 1, 2, 3, 4, 5, 6, 7, 8, 9, 10 or more non-negatively charged internucleotidic linkages.

121. The composition of any one of the preceding embodiments, wherein the oligonucleotides comprise a wing-core-wing, core-wing, or wing-core structure, and wherein a core comprises 1, 2, 3, 4, 5, 6, 7, 8, 9, 10 or more chirally controlled non-negatively charged internucleotidic linkages.

122. The composition of any one of the preceding embodiments, wherein the oligonucleotides comprise a wing-core-wing, core-wing, or wing-core structure, and wherein a core comprises 2, 3, 4, 5, 6, 7, 8, 9, 10 or more consecutive non-negatively charged internucleotidic linkages.

123. The composition of any one of the preceding embodiments, wherein the oligonucleotides comprise a wing-core-wing, core-wing, or wing-core structure, and wherein a core comprises 2, 3, 4, 5, 6, 7, 8, 9, 10 or more consecutive chirally controlled non-negatively charged internucleotidic linkages.

124. The composition of any one of the preceding embodiments, wherein 40%, 45%, 50%,

55%, 60%, 65%, 70%, 75%, 80%, 85%, 90%, 95%, or 100% of internucleotidic linkages of a wing is independently a non-negatively charged internucleotidic linkage, a natural phosphate internucleotidic linkage or a Rp chiral internucleotidic linkage.

125. The composition of any one of the preceding embodiments, wherein 40%, 45%, 50%,

55%, 60%, 65%, 70%, 75%, 80%, 85%, 90%, 95%, or 100% of internucleotidic linkages of a wing is independently a non-negatively charged internucleotidic linkage or a natural phosphate internucleotidic linkage.

126. The composition of any one of the preceding embodiments, wherein 40%, 45%, 50%,

55%, 60%, 65%, 70%, 75%, 80%, 85%, 90%, 95%, or 100% of internucleotidic linkages of a wing is independently a non-negatively charged internucleotidic linkage.

127. The composition of any one of embodiments 124-126, wherein the percentage is 50% or more.

128. The composition of any one of embodiments 124-126, wherein the percentage is 60% or more.

129. The composition of any one of embodiments 124-126, wherein the percentage is 75% or more.

130. The composition of any one of embodiments 124-126, wherein the percentage is 80% or more.

131. The composition of any one of embodiments 124-126, wherein the percentage is 90% or more.

132. The composition of any one of the preceding embodiments, wherein the oligonucleotides each comprise a non-negatively charged internucleotidic linkage and a natural phosphate internucleotidic linkage.

133. The composition of any one of the preceding embodiments, wherein the oligonucleotides each comprise a non-negatively charged internucleotidic linkage, a natural phosphate internucleotidic linkage and a Rp chiral internucleotidic linkage.

134. The composition of any one of the preceding embodiments, wherein a wing comprises a non-negatively charged internucleotidic linkage and a natural phosphate internucleotidic linkage.

135. The composition of any one of the preceding embodiments, wherein a wing comprises a non-negatively charged internucleotidic linkage, a natural phosphate internucleotidic linkage and a Rp chiral internucleotidic linkage.

136. The composition of any one of the preceding embodiments, wherein a core comprises 1, 2, 3, 4, 5, 6, 7, 8, 9, 10 or more non-negatively charged internucleotidic linkages.

137. The composition of any one of the preceding embodiments, wherein all non-negatively charged internucleotidic linkages of the same oligonucleotide have the same constitution.

138. The composition of any one of the preceding embodiments, wherein each of the non-negatively charged internucleotidic linkages independently has the structure of formula I-n-1, I-n-2, I-n-3, I-n-4, II, 11-a-1, II-a-2, 11-b-1, II-b-2, II-c-1, II-c-2, II-d-1, II-d-2, or a salt form thereof.

139. The composition of any one of the preceding embodiments, wherein each of the non-negatively charged internucleotidic linkages independently has the structure of formula I-n-1, I-n-2, I-n-3, I-n-4, II, 11-a-1, II-a-2, 11-b-1, II-b-2, II-c-1, II-c-2, II-d-1, II-d-2, or a salt form thereof.

140. The composition of any one of the preceding embodiments, wherein each of the non-negatively charged internucleotidic linkages independently has the structure of formula II, II-a- 1, II-a-2, II-b-1, II-b-2, II-c-1, II-c-2, II-d-1, II-d-2, or a salt form thereof.

141. The composition of any one of the preceding embodiments, wherein each of the non-negatively charged internucleotidic linkages independently has the structure of formula II, II-a-1, II-a-2, II-b-1, II-b-2, II-c-1, II-c-2, II-d-1, II-d-2, or a salt form thereof.

142. The composition of any one of the preceding embodiments, wherein the pattern of backbone linkages comprises at least one non-negatively charged internucleotidic linkage which is a neutral internucleotidic linkage.

143. The composition of any one of the preceding embodiments, wherein the pattern of backbone linkages comprises at least one neutral internucleotidic linkage which is or comprises a triazole, neutral triazole, alkyne, or a cyclic guanidine.

144. The composition of any one of the preceding embodiments, wherein the oligonucleotide type comprises any of: cholesterol; L-carnitine (amide and carbamate bond); Folic acid; Gambogic acid; Cleavable lipid (1,2-dilaurin and ester bond); Insulin receptor ligand; CPP: Glucose (tri- and hex-antennary); or Mannose (tri- and hex-antennary, alpha and beta).

145. The composition of any one of the preceding embodiments, wherein the oligonucleotide type is any oligonucleotide listed in Table A1.

146. The composition of any one of the preceding embodiments, wherein each of the oligonucleotides comprises a chemical moiety conjugated to the oligonucleotide chain of the oligonucleotide optionally through a linker moiety, wherein the chemical moiety comprises a carbohydrate moiety, a peptide moiety, a receptor ligand moiety, or a moiety having the structure of —N(R¹)₂, —N(R¹)₃, or —N═C(N(R¹)₂)₂.

147. The composition of any one of the preceding embodiments, wherein each of the oligonucleotides comprises a chemical moiety conjugated to the oligonucleotide chain of the oligonucleotide optionally through a linker moiety, wherein the chemical moiety comprises a guanidine moiety.

148. The composition of any one of the preceding embodiments, wherein each of the oligonucleotides comprises a chemical moiety conjugated to the oligonucleotide chain of the oligonucleotide optionally through a linker moiety, wherein the chemical moiety comprises —N═C(N(CH₃)₂)₂.

149. The composition of any one of the preceding embodiments, wherein at least 5%, 10%, 20%, 30%, 40%, 50%, 60%, 70%, 80%, or 90% of the oligonucleotides in the composition that have the base sequence of the particular oligonucleotide type are oligonucleotides of the particular oligonucleotide type.

150. The composition of any one of the preceding embodiments, wherein at least 5%, 10%, 20%, 30%, 40%, 50%, 60%, 70%, 80%, or 90% of the oligonucleotides in the composition that have the base sequence, pattern of backbone linkages, and pattern of backbone phosphorus modifications of the particular oligonucleotide type are oligonucleotides of the particular oligonucleotide type.

151. The composition of any one of the preceding embodiments, wherein the oligonucleotides of the particular type are structurally identical.

152. The composition of any one of the preceding embodiments, wherein a non-negatively charged internucleotidic linkage is a phosphoramidate linkage.

153. The composition of any one of the preceding embodiments, wherein a non-negatively charged internucleotidic linkage comprises a guanidine moiety.

154. The composition of any one of the preceding embodiments, wherein a non-negatively charged internucleotidic linkage has the structure of formula I:

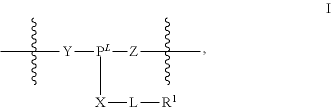

or a salt form thereof, wherein:
P^{L} is P(═W), P, or P—B(R)₃;
W is O, N(-L-R⁵), S or Se;
each of R¹ and R⁵ is independently —H, -L-R′, halogen, —CN, —NO₂, -L-Si(R′)₃, —OR′, —SR′, or —N(R′)₂;
each of X, Y and Z is independently —O—, —S—, —N(-L-R⁵)—, or L;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced with C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more CH or carbon atoms are optionally and independently replaced with Cy^{L};
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
each Cy^{L} is independently an optionally substituted trivalent or tetravalent group selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
each R′ is independently —R, —C(O)R, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or
two or more R groups on the same atom are optionally and independently taken together with the atom to form an optionally substituted, 3-30 membered, monocyclic, bicyclic or polycyclic ring having, in addition to the atom, 0-10 heteroatoms, or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

155. The composition of any one of the preceding embodiments, wherein each non-negatively charged internucleotidic linkage independently has the structure of formula I or a salt form thereof.

156. The composition of any one of the preceding embodiments, wherein a non-negatively charged internucleotidic linkage has the structure of formula I-n-1 or a salt form thereof:

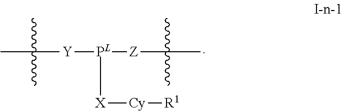

157. The composition of any one of the preceding embodiments, wherein each non-negatively charged internucleotidic linkage independently has the structure of formula I-n-1 or a salt form thereof.

158. The composition of any one of the preceding embodiments, wherein a non-negatively charged internucleotidic linkage has the structure of formula I-n-2 or a salt form thereof:

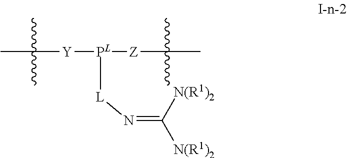

159. The composition of any one of the preceding embodiments, wherein a non-negatively charged internucleotidic linkage has the structure of formula I-n-3 or a salt form thereof:

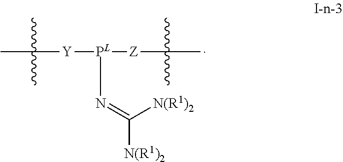

160. The composition of any one of the preceding embodiments, wherein each non-negatively charged internucleotidic linkage independently has the structure of formula I-n-3 or a salt form thereof.

161. The composition of any one of the preceding embodiments, wherein a non-negatively charged internucleotidic linkage has the structure of formula I-n-3 or a salt form thereof, wherein one R′ from one —N(R′)₂ and one R′ from the other —N(R′)₂ are taken together with their intervening atoms to form an optionally substituted, 3-30 membered, monocyclic, bicyclic or polycyclic ring having, in addition to the intervening atoms, 0-10 heteroatoms.

162. The composition of any one of the preceding embodiments, wherein each non-negatively charged internucleotidic linkage independently has the structure of formula I-n-3 or a salt form thereof, wherein one R′ from one —N(R′)₂ and one R′ from the other —N(R′)₂ are taken together with their intervening atoms to form an optionally substituted, 3-30 membered, monocyclic, bicyclic or polycyclic ring having, in addition to the intervening atoms, 0-10 heteroatoms.

163. The composition of any one of the preceding embodiments, wherein a non-negatively charged internucleotidic linkage has the structure of formula I-n-3 or a salt form thereof, wherein one R′ from one —N(R′)₂ and one R′ from the other —N(R′)₂ are taken together with their intervening atoms to form an optionally substituted 5-membered monocyclic ring having no more than two nitrogen atoms.

164. The composition of any one of the preceding embodiments, wherein each non-negatively charged internucleotidic linkage independently has the structure of formula I-n-3 or a salt form thereof, wherein one R′ from one —N(R′)₂ and one R′ from the other —N(R′)₂ are taken together with their intervening atoms to form an optionally substituted 5-membered monocyclic ring having no more than two nitrogen atoms.

165. The composition of any one of embodiments 159-162, wherein the ring formed is a saturated ring.

166. The composition of any one of embodiments 159-162, wherein the ring formed is a partially unsaturated ring.

167. The composition of any one of the preceding embodiments, wherein a non-negatively charged internucleotidic linkage has the structure of formula I-n-4 or a salt form thereof:

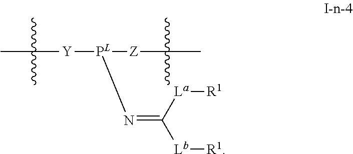

168. The composition of embodiment 167, wherein L^{a} is a covalent bond.

169. The composition of embodiment 167, wherein L^{a} is —N(R¹)—.

170. The composition of embodiment 167, wherein L^{a} is —N(R′)—.

171. The composition of embodiment 167, wherein L^{a} is —N(R)—.

172. The composition of embodiment 167, wherein L^{a} is —S(O)—.

173. The composition of embodiment 167, wherein L^{a} is —S(O)₂—.

174. The composition of embodiment 167, wherein L^{a} is —S(O)₂N(R′)—.

175. The composition of any one of embodiments 167-174, wherein L^{b} is a covalent bond.

176. The composition of any one of embodiments 167-174, wherein L^{b} is —N(R¹)—.

177. The composition of any one of embodiments 167-174, wherein L^{b} is —N(R′)—.

178. The composition of any one of embodiments 167-174, wherein L^{b} is —N(R)—.

179. The composition of any one of embodiments 167-174, wherein L^{b} is —S(O)—.

180. The composition of any one of embodiments 167-174, wherein L^{b} is —S(O)₂—.

181. The composition of any one of embodiments 167-174, wherein L^{b} is —S(O)₂N(R′)—.

182. The composition of any one of the preceding embodiments, wherein a non-negatively charged internucleotidic linkage has the structure of formula II:

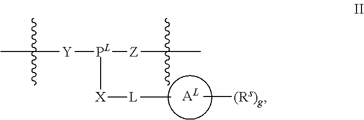

or a salt form thereof, wherein:
P^{L} is P(═W), P, or P→B(R′)₃;
W is O, N(-L-R⁵), S or Se;
each of X, Y and Z is independently —O—, —S—, —N(-L-R⁵)—, or L;
R⁵ is —H, -L-R′, halogen, —CN, —NO₂, -L-Si(R′)₃, —OR′, —SR′, or —N(R)₂;
Ring A^{L} is an optionally substituted 3-20 membered monocyclic, bicyclic or polycyclic ring having 0-10 heteroatoms;
each R^{s} is independently —H, halogen, —CN, —N₃, —NO, —NO₂, -L-R′, -L-Si(R)₃, -L-OR′, -L-SR′, -L-N(R′)₂, —O-L-R′, —O-L-Si(R)₃, —O-L-OR′, —O-L-SR′, or —O-L-N(R′)₂;
g is 0-20;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced with C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more CH or carbon atoms are optionally and independently replaced with Cy^{L};
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
each Cy^{L} is independently an optionally substituted trivalent or tetravalent group selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms:
each R′ is independently —R, —C(O)R, —C(O)OR, or —S(O)₂R;
each R is independently —H. or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or
two or more R groups on the same atom are optionally and independently taken together with the atom to form an optionally substituted, 3-30 membered, monocyclic, bicyclic or polycyclic ring having, in addition to the atom, 0-10 heteroatoms, or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

183. The composition of any one of the preceding embodiments, wherein each non-negatively charged internucleotidic linkage independently has the structure of formula II, or a salt form thereof.

184. The composition any one of the preceding embodiments, wherein a non-negatively charged internucleotidic linkage has the structure of formula II-a-1:

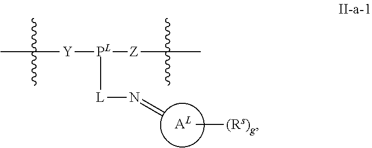

or a salt form thereof.

185. The composition any one of the preceding embodiments, wherein a non-negatively charged internucleotidic linkage has the structure of formula II-a-2:

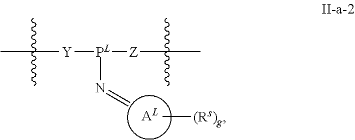

or a salt form thereof.

186. The composition of any one of the preceding embodiments, wherein each non-negatively charged internucleotidic linkage independently has the structure of formula II-a-1 or II-a-2, or a salt form thereof.

187. The composition of any one of embodiments 182-186, wherein a non-negatively charged internucleotidic linkage has the structure of formula II-b-1:

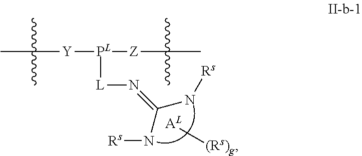

or a salt form thereof, wherein g is 0-18.

188. The composition of any one of embodiments 182-187, wherein a non-negatively charged internucleotidic linkage has the structure of formula II-b-2:

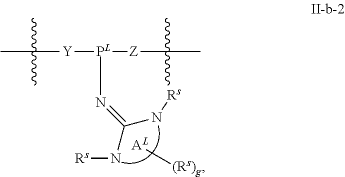

or a salt form thereof, wherein g is 0-18.

189. The composition of any one of the preceding embodiments, wherein each non-negatively charged internucleotidic linkage independently has the structure of formula II-b-1 or II-b-2, or a salt form thereof.

190. The composition of any one of embodiments 182-188, wherein Ring A^{L} is an optionally substituted 3-20 membered monocyclic ring having 0-10 heteroatoms (in addition to the two nitrogen atoms for formula II-b-1 or II-b-2).

191. The composition of any one of embodiments 182-188, wherein Ring A^{L} is an optionally substituted 5-membered monocyclic saturated ring.

192. The composition of any one of embodiments 182-191, wherein a non-negatively charged internucleotidic linkage has the structure of formula II-c-1:

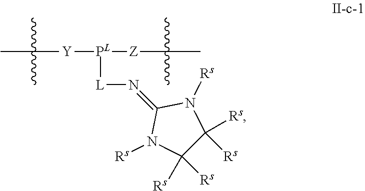

or a salt form thereof, wherein g is 04.

193. The composition of any one of embodiments 182-193, wherein a non-negatively charged internucleotidic linkage has the structure of formula II-c-2:

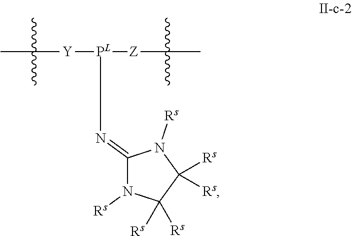

or a salt form thereof, wherein g is 04.

194. The composition of any one of the preceding embodiments, wherein each non-negatively charged internucleotidic linkage independently has the structure of formula II-c-1 or II-c-2, or a salt form thereof.

195. The composition of any one of embodiments 182-193, wherein each non-negatively charged internucleotidic linkage has the same structure.

196. The composition of any one of the preceding embodiments, wherein, if applicable, each internucleotidic linkage in the oligonucleotides of the plurality that is not a non-negatively charged internucleotidic linkage independently has the structure of formula I.

197. The composition of any one of the preceding embodiments, wherein each internucleotidic linkage in the oligonucleotides of the plurality independently has the structure of formula I.

198. The composition of any one of the preceding embodiments, wherein one or more P^{L} is P(═W).

199. The composition of any one of the preceding embodiments, wherein each P^{L} is independently P(═W).

200. The composition of any one of the preceding embodiments, wherein one or more W is O.

201. The composition of any one of the preceding embodiments, wherein each W is O.

202. The composition of any one of the preceding embodiments, wherein one or more W is S.

203. The composition of any one of the preceding embodiments, wherein one or more W is independently N(-L-R⁵).

204. The composition of any one of the preceding embodiments, wherein one or more internucleotidic linkage independently has the structure of formula III or salt form thereof:

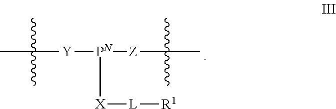

205. The composition of embodiment 204, wherein P^{N} is P(═N-L-R⁵).

206. The composition of embodiment 204, wherein P^{N} is

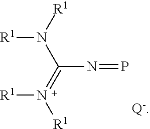

207. The composition of embodiment 204, wherein P^{N} is

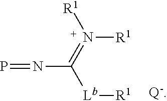

208. The composition of embodiment 207, wherein L^{a} is a covalent bond.

209. The composition of embodiment 207, wherein L^{a} is —N(R¹)—.

210. The composition of embodiment 207, wherein L^{a} is —N(R′)—.

211. The composition of embodiment 207, wherein L^{a} is —N(R)—.

212. The composition of embodiment 207, wherein U is —S(O)—.

213. The composition of embodiment 207, wherein L^{a} is —S(O)₂—.

214. The composition of embodiment 207, wherein L^{a} is —S(O)₂N(R′)—.

215. The composition of embodiment 204, wherein P^{N} is

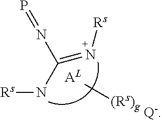

216. The composition of embodiment 204, wherein P^{N} is

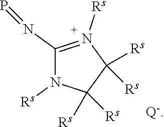

217. The composition of embodiment 204, wherein P^{N} is

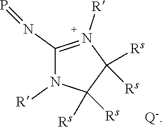

218. The composition of any one of the preceding embodiments, wherein one or more Y is O.

219. The composition of any one of the preceding embodiments, wherein each Y is O.

220. The composition of any one of the preceding embodiments, wherein one or more Z is O.

221. The composition of any one of the preceding embodiments, wherein each Z is O.

222. The composition of any one of the preceding embodiments, wherein one or more X is O.

223. The composition of any one of the preceding embodiments, wherein one or more X is S.

224. The composition of any one of the preceding embodiments, wherein a non-negatively charged internucleotidic linkage has the structure of

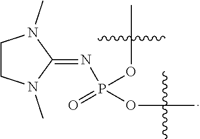

225. The composition of any one of the preceding embodiments, wherein a non-negatively charged internucleotidic linkage has the structure of

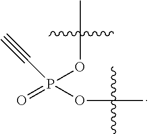

226. The composition of any one of the preceding embodiments, wherein a non-negatively charged internucleotidic linkage has the structure of

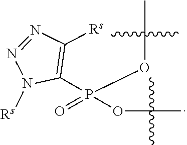

227. The composition of any one of the preceding embodiments, wherein for each internucleotidic linkage of formula I or a salt fore thereof that is not a non-negatively charged internucleotidic linkage, X is independently O or S, and -L-R¹ is —H (natural phosphate linkage or phosphorothioate linkage, respectively).

228. The composition of any one of the preceding embodiments, wherein each phosphorothioate linkage, if any, in the oligonucleotides of the plurality is independently a chirally controlled internucleotidic linkage.

229. The composition of any one of the preceding embodiments, wherein at least one non-negatively charged internucleotidic linkage is a chirally controlled internucleotidic linkage.

230. The composition of any one of the preceding embodiments, wherein at least one non-negatively charged internucleotidic linkage is a chirally controlled internucleotidic linkage.

231. The composition of any one of the preceding embodiments, wherein the oligonucleotides of the plurality comprise a targeting moiety wherein the targeting moiety is independently connected to an oligonucleotide backbone through a linker.

232. The composition of embodiment 231, wherein the targeting moiety is a carbohydrate moiety.

233. The composition of embodiment 231 or 232, wherein the targeting moiety comprises or is a GalNac moiety.

234. The composition of any one of the preceding embodiments, wherein the oligonucleotides of the plurality comprise a lipid moiety wherein the lipid moiety is independently connected to an oligonucleotide backbone through a linker.

235. The composition of any one of the preceding embodiments, wherein oligonucleotides of the plurality exist as salts, wherein one or more non-neutral internucleotidic linkages at the condition of the composition independently exist as a salt form.

236. The composition of any one of the preceding embodiments, wherein oligonucleotides of the plurality exist as salts, wherein one or more negatively-charged internucleotidic linkages at the condition of the composition independently exist as a salt form.

237. The composition of any one of the preceding embodiments, wherein oligonucleotides of the plurality exist as salts, wherein one or more negatively-charged internucleotidic linkages at the condition of the composition independently exist as a metal salt.

238. The composition of any one of the preceding embodiments, wherein oligonucleotides of the plurality exist as salts, wherein each negatively-charged internucleotidic linkage at the condition of the composition independently exists as a metal salt.

239. The composition of any one of the preceding embodiments, wherein oligonucleotides of the plurality exist as salts, wherein each negatively-charged internucleotidic linkage at the condition of the composition independently exists as sodium salt.

240. The composition of any one of the preceding embodiments, wherein oligonucleotides of the plurality exist as salts, wherein each negatively-charged internucleotidic linkage is independently a natural phosphate linkage (the neutral form of which is —O—P(O)(OH)—O) or phosphorothioate internucleotidic linkage (the neutral form of which is —O—P(O)(SH)—O).

241. An oligonucleotide composition, comprising a plurality of oligonucleotides of a particular oligonucleotide type defined by:
1) base sequence;
2) pattern of backbone linkages:
3) pattern of backbone chiral centers; and
4) pattern of backbone phosphorus modifications,
   wherein:
oligonucleotides of the plurality comprise at least 1, 2, 3, 4, 5, 6, 7, 8, 9, 10, 11, 12, 13, 14, 15, 16, 17, 18, 19, or 20 chirally controlled internucleotidic linkages; and oligonucleotides of the plurality comprise at least 1, 2, 3, 4, 5, 6, 7, 8, 9, 10, 11, 12, 13, 14, 15, 16, 17, 18, 19, or 20 non-negatively charged internucleotidic linkages.

242. The composition of any one of the preceding embodiments, wherein at least one non-negatively charged internucleotidic linkage is a neutral internucleotidic linkage.

243. The composition of any one of the preceding embodiments, wherein a neutral internucleotidic linkage is or comprises a triazole, neutral triazole, alkyne, or a cyclic guanidine.

244. The oligonucleotide composition of any one of the preceding embodiments, wherein the oligonucleotide composition is characterized in that, when it is contacted with a transcript in a transcript splicing system, splicing of the transcript is altered relative to that observed under a reference condition selected from the group consisting of absence of the composition, presence of a reference composition, and combinations thereof.

245. The oligonucleotide composition of any one of the preceding embodiments, wherein the transcript is a Dystrophin transcript.

246. The oligonucleotide composition of any one of the preceding embodiments, wherein the splicing of the transcript is altered such that the level of skipping of exon 45, 51, or 53, or multiple exons is increased.

247. The oligonucleotide composition of any one of the preceding embodiments, wherein the oligonucleotide composition is capable of mediating knockdown of a target gene.

248. An oligonucleotide composition, comprising a plurality of oligonucleotides of a particular oligonucleotide type defined by:
1) base sequence;
2) pattern of backbone linkages;
3) pattern of backbone chiral centers; and
4) pattern of backbone phosphorus modifications,
   wherein:
the oligonucleotides of the plurality comprise cholesterol; L-carnitine (amide and carbamate bond); Folic acid: Cleavable lipid (1,2-dilaurin and ester bond); Insulin receptor ligand; Gambogic acid; CPP; Glucose (tri- and hex-antennary); or Mannose (tri- and hex-antennary, alpha and beta).

249. The composition of embodiment 248, wherein the oligonucleotides of the plurality comprise at least 1, 2, 3, 4, 5, 6, 7, 8, 9, 10, 11, 12, 13, 14, 15, 16, 17, 18, 19, or 20 chirally controlled internucleotidic linkages.

250. The composition of any one of the preceding embodiments, wherein the oligonucleotide composition is characterized in that, when it is contacted with a transcript in a transcript splicing system, splicing of the transcript is altered relative to that observed under a reference condition selected from the group consisting of absence of the composition, presence of a reference composition, and combinations thereof.

251. The composition of any one of the preceding embodiments, wherein the transcript is a Dystrophin transcript.

252. The composition of any one of the preceding embodiments, wherein the splicing of the transcript is altered such that the level of skipping of exon 45, 51, or 53, or multiple exons is increased.

253. The composition of any one of the preceding embodiments, wherein the oligonucleotide composition is capable of mediating knockdown of a target gene.

254. An oligonucleotide composition comprising a plurality of oligonucleotides, wherein oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”).

255. The composition of embodiment 254, wherein oligonucleotides of the plurality share the same linkage phosphorus stereochemistry independently at five or more internucleotidic linkages.

256. The composition of embodiment 254, wherein oligonucleotides of the plurality share the same linkage phosphorus stereochemistry independently at five or more phosphorothioate internucleotidic linkages.

257. The composition of any one of embodiments 254-256, wherein oligonucleotides of the plurality share the same linkage phosphorus stereochemistry independently at each phosphorothioate internucleotidic linkages.

258. The composition of any one of embodiments 254-256, wherein oligonucleotides of the plurality share the same linkage phosphorus stereochemistry independently at each chiral internucleotidic linkages.

259. The composition of any one of embodiments 254-258, wherein oligonucleotides of the plurality comprise one or more (e.g., 1, 2, 3, 4, 5, 6, 7, 8, 9, or 10) neutral internucleotidic linkages.

260. The composition of any one of embodiments 254-258, wherein oligonucleotides of the plurality comprise one or more (e.g., 1, 2, 3, 4, 5, 6, 7, 8, 9, or 10) n001.

261. The composition of any one of embodiments 254-260, wherein oligonucleotides of the plurality comprise one or more (e.g., 1, 2, 3, 4, 5, 6, 7, 8, 9, or 10) natural phosphate linkages.

262. The composition of any one of embodiments 254-261, wherein the base sequence is or comprises UCAAGGAAGAUGGCAUUUCU (SEQ ID NO: 406).

263. The composition of any one of embodiments 254-261, wherein the base sequence is or comprises GGUAAGUUCUGUCCAAGCCC (SEQ ID NO: 394).

264. The composition of any one of embodiments 254-261, wherein the base sequence is or comprises GUACCUCCAACAUCAAGGAA (SEQ ID NO: 399).

265. The composition of any one of embodiments 254-261, wherein the base sequence is or comprises CAACAUCAAGGAAGAUGGCA (SEQ ID NO: 407).

266. The composition of any one of embodiments 254-261, wherein the base sequence is or comprises GAUGGCAUUUCUAGUUUGGA (SEQ ID NO: 408).

267. The composition of any one of embodiments 254-261, wherein the base sequence is or comprises AUGGCAUUUCUAGUUUGGAG (SEQ ID NO: 395).

268. The composition of any one of embodiments 254-261, wherein the base sequence is or comprises UGGCAUUUCUAGUUUGGAGA (SEQ ID NO: 393).

269. The composition of any one of embodiments 254-261, wherein the base sequence is or comprises GGCAUUUCUAGUUUGGAGAU (SEQ ID NO: 400).

270. The composition of any one of embodiments 254-261, wherein the base sequence is or comprises GCAUUUCUAGUUUGGAGAUG (SEQ ID NO: 396).

271. The composition of any one of embodiments 254-261, wherein the base sequence is or comprises GCAGUUUCCUUAGUAACCAC (SEQ ID NO: 409).

272. The composition of any one of embodiments 254-261, wherein the base sequence is or comprises CAGUUUCCUUAGUAACCACA (SEQ ID NO: 397).

273. The composition of any one of embodiments 254-261, wherein the base sequence is or comprises UUCCUUAGUAACCACAGGUU (SEQ ID NO: 398).

274. The composition of any one of embodiments 254-261, wherein the base sequence is or comprises UUGUGUCACCAGAGUAACAG (SEQ ID NO: 410).

275. The composition of any one of embodiments 254-261, wherein the base sequence is or comprises UGGCAGUUUCCUUAGUAACC (SEQ ID NO: 401).

276. The composition of any one of embodiments 254-261, wherein the base sequence is or comprises AGUUUCCUUAGUAACCACAG (SEQ ID NO: 392).

277. The composition of any one of the preceding embodiments, wherein at least 5%, 10%, 20%, 30%, 40%, 50%, 60%, 70%, 80%, or 90% of the oligonucleotides in the composition that have the base sequence of the oligonucleotides of the plurality are oligonucleotides of the plurality.

278. The composition of any one of the preceding embodiments, wherein at least 5%, 10%, 20%, 30%, 40%, 50%, 60%, 70%, 80%, or 90% of the oligonucleotides in the composition that have the base sequence, pattern of backbone linkages, and pattern of backbone phosphorus modifications of the oligonucleotides of plurality are oligonucleotides of the plurality.

279. The composition of any one of the preceding embodiments, wherein oligonucleotides of the plurality have the same constitution, and at least 5%, 10%, 20%, 30%, 40%, 50%, 60%, 70%, 80%, or 90% of the oligonucleotides in the composition that have the same constitution are oligonucleotides of the plurality.

280. The composition of any one of the preceding embodiments, wherein each heteroatom is independently boron, nitrogen, oxygen, silicon, sulfur, or phosphorus.

281. A pharmaceutical composition comprising an oligonucleotide composition of any one of the preceding embodiments and a pharmaceutically acceptable carrier.

282. A composition, wherein a level of all oligonucleotides in the composition are oligonucleotides each independently having the structure of:
fG*SfU*SfAn001RfC*SfC*SfUn001RfC*SfC*SmAfA*SmC*SfA*SmUfC*SfA*SfA*SfGn001RfG*SfA*SfA (SEQ ID NO: 172), or a pharmaceutically acceptable salt thereof, wherein:
f represents a 2-F modified nucleoside;
*S represents a Sp phosphorothioate;
m represents a 2′-OMe modified nucleoside; and
n001R is

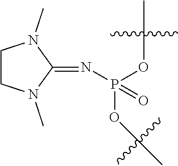 [SMILES: *OP(=O)(N=C1N(C)CCN1C)O*]

wherein the phosphorus is of the Rp configuration.

283. A composition, wherein a level of all oligonucleotides in the composition that have the base sequence of GUACCUCCAACAUCAAGGAA (SEQ ID NO: 411) are oligonucleotides each independently having the structure of:
fG*SfU*SfAn001RfC*SfC*SfUn001RfC*SfC*SmAfA*SmC*SfA*SmUfC*SfA*SfA*SfGn001RfG*SfA*SfA (SEQ ID NO: 172), or a pharmaceutically acceptable salt thereof, wherein:
f represents a 2-F modified nucleoside;
*S represents a Sp phosphorothioate;
m represents a 2′-OMe modified nucleoside; and
n001R is

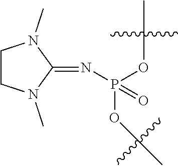 [SMILES: *OP(=O)(N=C1N(C)CCN1C)O*]

wherein the phosphorus is of the Rp configuration.

284. A composition, wherein a level of all oligonucleotides in the composition are oligonucleotides each independently having the structure of:
fU*SfG*SfGn001RfC*SfA*SfUn001RfU*SfU*SmCfU*SmA*SfG*SmUfU*SfU*SfG*SfGn001RfA*SfG*SfA (SEQ ID NO: 175), or a pharmaceutically acceptable salt thereof, wherein:
f represents a 2-F modified nucleoside;
*S represents a Sp phosphorothioate;
m represents a 2′-OMe modified nucleoside; and
n001R is

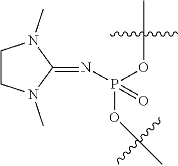 [SMILES: *OP(=O)(N=C1N(C)CCN1C)O*]

wherein the phosphorus is of the Rp configuration.

285. A composition, wherein a level of all oligonucleotides in the composition that have the base sequence of UGGCAUUUCUAGUUUGGAGA (SEQ ID NO: 412) are oligonucleotides each independently having the structure of:
fU*SfG* SfGn001RfC* SfA*SfUn001RfU* SfU*SmCfU*SmA*SfG*SmUfU* SfU* SfG*SfGn001 RfA*SfG*SfA (SEQ ID NO: 175), or a pharmaceutically acceptable salt thereof, wherein:
f represents a 2′-F modified nucleoside;
*S represents a Sp phosphorothioate;
m represents a 2′-OMe modified nucleoside; and
n001R is

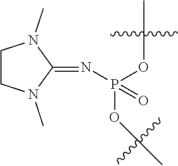 [SMILES: *OP(=O)(N=C1N(C)CCN1C)O*]

wherein the phosphorus is of the Rp configuration.

286. A composition, wherein a level of all oligonucleotides in the composition are oligonucleotides each independently having the structure of:
fG*SfG*SfCn001RfA*SfU*SfUn001RmUfC*SfU*SmA*SfG*SmUmU*SfU*SfG*SfG*SfAn001RfG*SfA*SfU (SEQ ID NO: 188), or a pharmaceutically acceptable salt thereof, wherein:
f represents a 2′-F modified nucleoside;
*S represents a Sp phosphorothioate;
m represents a 2′-OMe modified nucleoside; and
n001R is

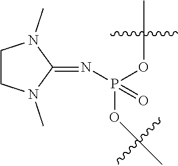 [SMILES: *OP(=O)(N=C1N(C)CCN1C)O*]

wherein the phosphorus is of the Rp configuration.

287. A composition, wherein a level of all oligonucleotides in the composition that have the base sequence of GGCAUUUCUAGUUUGGAGAU (SEQ ID NO: 413) are oligonucleotides each independently having the structure of:
fG*SfG*SfCn001RfA*SfU*SfUn001RmUfC*SfU*SmA*SfG*SmUmU*SfU*SfG*SfG*SfAn001RfG*SfA*SfU (SEQ ID NO: 188), or a pharmaceutically acceptable salt thereof, wherein:
f represents a 2′-F modified nucleoside;
*S represents a Sp phosphorothioate;
m represents a 2′-OMe modified nucleoside; and
n001R is
f represents a 2′-F modified nucleoside;
*S represents a Sp phosphorothioate;
m represents a 2′-OMe modified nucleoside; and

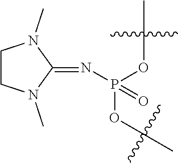 [SMILES: *OP(=O)(N=C1N(C)CCN1C)O*]

wherein the phosphorus is of the Rp configuration.

288. A composition, wherein a level of all oligonucleotides in the composition are oligonucleotides each independently having the structure of:
fU*SfG*SfGn001RfC*SfA*SfGn001RfU*SfU*SmUfC*SmC*SfU*SmUfA*SfG*SfU*SfAn00 RfA*SfC*SfC (SEQ ID NO: 178), or a pharmaceutically acceptable salt thereof, wherein:
f represents a 2′-F modified nucleoside;
*S represents a Sp phosphorothioate;
m represents a 2′-OMe modified nucleoside; and
n001R is

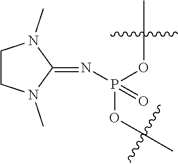 [SMILES: *OP(=O)(N=C1N(C)CCN1C)O*]

wherein the phosphorus is of the Rp configuration.

289. A composition, wherein a level of all oligonucleotides in the composition that have the base sequence of UGGCAGUUUCCUUAGUAACC (SEQ ID NO: 414) are oligonucleotides each independently having the structure of:
fU*SfG*SfGn001RfC*SfA*SfGn001RfU*SfU*SmUfC*SmC*SfU*SmUfA*SfG*SfU*SfAn001RfA*SfC*SfC (SEQ ID NO: 178), or a pharmaceutically acceptable salt thereof, wherein:
f represents a 2′-F modified nucleoside;
*S represents a Sp phosphorothioate;
m represents a 2′-OMe modified nucleoside; and
n001R is

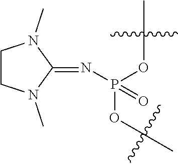 [SMILES: *OP(=O)(N=C1N(C)CCN1C)O*]

wherein the phosphorus is of the Rp configuration.

290. A composition, wherein a level of all oligonucleotides in the composition are oligonucleotides each independently having the structure of:
fU*SfG*SfGn001RfC*SfA*SfGn001RmUfU* SfU*SmC*SfC*SmUmU*SfA*SfG*SfU*SfAn001RfA*SfC*SfC (SEQ ID NO: 190), or a pharmaceutically acceptable salt thereof, wherein:
f represents a 2′-F modified nucleoside;
*S represents a Sp phosphorothioate;
m represents a 2′-OMe modified nucleoside; and
n001R is

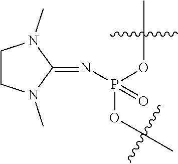 [SMILES: *OP(=O)(N=C1N(C)CCN1C)O*]

wherein the phosphorus is of the Rp configuration.

291. A composition, wherein a level of all oligonucleotides in the composition that have the base sequence of UGGCAGUUUCCUUAGUAACC (SEQ ID NO: 415) are oligonucleotides each independently having the structure of:
fU*SfG*SfGn001RfC*SfA*SfGn001RmUfU*SfU*SmC*SfC*SmUmU*SfA*SfG*SfU*SfAn001RfA*SfC*SfC (SEQ ID NO: 190), or a pharmaceutically acceptable salt thereof, wherein:
f represents a 2′-F modified nucleoside;
*S represents a Sp phosphorothioate;
m represents a 2′-OMe modified nucleoside; and
n001R is

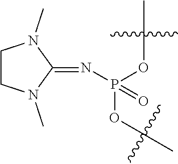 [SMILES: *OP(=O)(N=C1N(C)CCN1C)O*]

wherein the phosphorus is of the Rp configuration.

292. A composition, wherein a level of all oligonucleotides in the composition are oligonucleotides each independently having the structure of:
fG* SfG*SfUn001RfA*SfA*SfGn001RfU* SfU*SmCfU*SmG* SfU*SmCfC*SfA*SfA*SfGn001RfC*SfC*SfC (SEQ ID NO: 171), or a pharmaceutically acceptable salt thereof, wherein:
f represents a 2′-F modified nucleoside;
*S represents a Sp phosphorothioate;
m represents a 2′-OMe modified nucleoside; and
n001R is

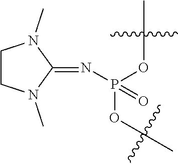 [SMILES: *OP(=O)(N=C1N(C)CCN1C)O*]

wherein the phosphorus is of the Rp configuration.

293. A composition, wherein a level of all oligonucleotides in the composition that have the base sequence of GGUAAGUUCUGUCCAAGCCC (SEQ ID NO: 416) are oligonucleotides each independently having the structure of:
fG* SfG*SfUn001RfA*SfA*SfGn001RfU* SfU*SmCfU*SmG* SfU*SmCfC*SfA*SfA*SfGn001RfC*SfC*SfC (SEQ ID NO: 171), or a pharmaceutically acceptable salt thereof, wherein:
f represents a 2′-F modified nucleoside;
*S represents a Sp phosphorothioate;
m represents a 2′-OMe modified nucleoside; and
n001R is

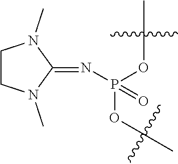 [SMILES: *OP(=O)(N=C1N(C)CCN1C)O*]

wherein the phosphorus is of the Rp configuration.

294. A composition, wherein a level of all oligonucleotides in the composition are oligonucleotides each independently having the structure of:
fG*SfG*SfUn001RfA*SfA*SfGn001 RmUfU*SfC*SmU*SfG*SmUmC*SfC*SfA*SfA*SfGn001RfC*SfC*SfC (SEQ ID NO: 183), or a pharmaceutically acceptable salt thereof, wherein:
f represents a 2′-F modified nucleoside;
*S represents a Sp phosphorothioate;
m represents a 2′-OMe modified nucleoside; and
n001R is

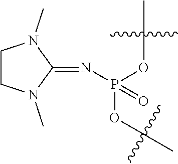 [SMILES: *OP(=O)(N=C1N(C)CCN1C)O*]

wherein the phosphorus is of the Rp configuration.

295. A composition, wherein a level of all oligonucleotides in the composition that have the base sequence of GGUAAGUUCUGUCCAAGCCC (SEQ ID NO: 417) are oligonucleotides each independently having the structure of:
fG*SfG*SfUn001RfA*SfA*SfGn001RmUfU*SfC*SmU*SfG*SmUmC*SfC*SfA*SfA*SfGn001RfC*SfC*SfC (SEQ ID NO: 183), or a pharmaceutically acceptable salt thereof, wherein:
f represents a 2′-F modified nucleoside;
*S represents a Sp phosphorothioate;
m represents a 2′-OMe modified nucleoside; and
n001R is

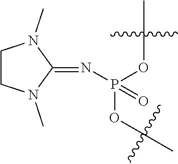 [SMILES: *OP(=O)(N=C1N(C)CCN1C)O*]

wherein the phosphorus is of the Rp configuration.

296. A composition, wherein a level of all oligonucleotides in the composition are oligonucleotides each independently having the structure of:
fC*SfA* SfAn0011RfC*SfA*SfUn001RC*SfA*SmAfG*SmG*SfA*SmAfG*SfA*SfU*SfGn001RfG*SfC*SfA (SEQ ID NO: 173), or a pharmaceutically acceptable salt thereof, wherein:
f represents a 2′-F modified nucleoside;
*S represents a Sp phosphorothioate;
m represents a 2′-OMe modified nucleoside; and
n001R is

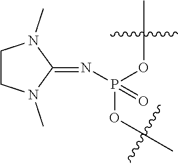 [SMILES: *OP(=O)(N=C1N(C)CCN1C)O*]

wherein the phosphorus is of the Rp configuration.

297. A composition, wherein a level of all oligonucleotides in the composition that have the base sequence of CAACAUCAAGGAAGAUGGCA (SEQ ID NO: 418) are oligonucleotides each independently having the structure of:
fC*SfA*SfAn001RfC*SfA*SfUn001RfC*SfA*SmAfG*SmG*SfA*SmAfG*SfA*SfU*SfGn001RfG*SfC*SfA (SEQ ID NO: 173), or a pharmaceutically acceptable salt thereof, wherein:
f represents a 2′-F modified nucleoside;
*S represents a Sp phosphorothioate;
m represents a 2′-OMe modified nucleoside; and
n001R is

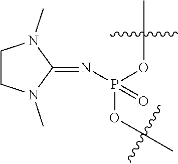 [SMILES: *OP(=O)(N=C1N(C)CCN1C)O*]

wherein the phosphorus is of the Rp configuration.

298. A composition, wherein a level of all oligonucleotides in the composition are oligonucleotides each independently having the structure of:
fA*SfU*SfGn001RfG*SfC*SfAn001RfU*SfU*SmUfC*SmU*SfA*SmGfU*SfU*SfU*SfGn001RfG*SfA*SfG (SEQ ID NO: 174), or a pharmaceutically acceptable salt thereof, wherein:
f represents a 2′-F modified nucleoside;
*S represents a Sp phosphorothioate;
m represents a 2′-OMe modified nucleoside; and
n001R is

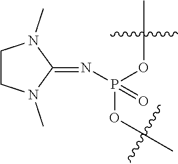 [SMILES: *OP(=O)(N=C1N(C)CCN1C)O*]

10 wherein the phosphorus is of the Rp configuration.

299. A composition, wherein a level of all oligonucleotides in the composition that have the base sequence of AUGGCAUUUCUAGUUUGGAG (SEQ ID NO: 419) are oligonucleotides each independently having the structure of:
fA*SfU*SfGn001RfG*SfC*SfAn001RfU*SfU*SmUfC*SmU*SfA*SmGfU*SfU*SfU*SfGn001RfG*SfA*SfG (SEQ ID NO: 174), or a pharmaceutically acceptable salt thereof, wherein:
f represents a 2′-F modified nucleoside;
*S represents a Sp phosphorothioate;
m represents a 2′-OMe modified nucleoside; and
n001R is

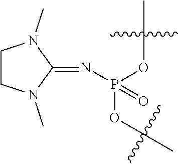 [SMILES: *OP(=O)(N=C1N(C)CCN1C)O*]

wherein the phosphorus is of the Rp configuration.

300. A composition, wherein a level of all oligonucleotides in the composition are oligonucleotides each independently having the structure of:
fA*SfU*SfGn001RfG*SfC*SfAn001RmUfU*SfU*SmC*SfU*SmAmG*SfU*SfU*SfU*SfGn001RfG*SfA*SfG (SEQ ID NO: 186), or a pharmaceutically acceptable salt thereof, wherein:
f represents a 2′-F modified nucleoside;
*S represents a Sp phosphorothioate;
m represents a 2′-OMe modified nucleoside; and
n001R is

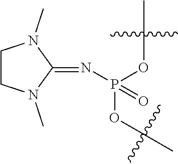 [SMILES: *OP(=O)(N=C1N(C)CCN1C)O*]

wherein the phosphorus is of the Rp configuration.

301. A composition, wherein a level of all oligonucleotides in the composition that have the base sequence of AUGGCAUUUCUAGUUUGGAG (SEQ ID NO: 420) are oligonucleotides each independently having the structure of:
fA*SfU*SfGn001RfG*SfC*SfAn001RmUfU*SfU*SmC*SfU*SmAmG*SfU*SfU*SfU*SfGn001RfG*SfA*SfG (SEQ ID NO: 186), or a pharmaceutically acceptable salt thereof, wherein:
f represents a 2′-F modified nucleoside;
*S represents a Sp phosphorothioate;
m represents a 2′-OMe modified nucleoside; and
n001R is

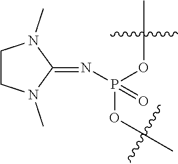 [SMILES: *OP(=O)(N=C1N(C)CCN1C)O*]

wherein the phosphorus is of the Rp configuration.

302. A composition, wherein a level of all oligonucleotides in the composition are oligonucleotides each independently having the structure of:
fG*SfC*SfAn001RfU*SfU*SfUn001RfC*SfU*SmAfG*SmU*SfU*SmUfG*SfG*SfA*SfGn001RfA*SfU*SfG (SEQ ID NO: 177), or a pharmaceutically acceptable salt thereof, wherein:
f represents a 2′-F modified nucleoside;
*S represents a Sp phosphorothioate;
m represents a 2′-OMe modified nucleoside; and
n001R is

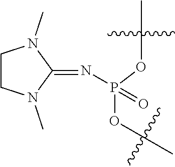 [SMILES: *OP(=O)(N=C1N(C)CCN1C)O*]

wherein the phosphorus is of the Rp configuration.

303. A composition, wherein a level of all oligonucleotides in the composition that have the base sequence of GCAUUUCUAGUUUGGAGAUG (SEQ ID NO: 421) are oligonucleotides each independently having the structure of:
fG*SfC*SfAn001RfU*SfU*SfUn001RfC*SfU*SmAfG*SmU*SfU*SmUfG*SfG*SfA*SfGn001 RfA*SfU*SfG (SEQ ID NO: 177), or a pharmaceutically acceptable salt thereof, wherein:
f represents a 2′-F modified nucleoside;
*S represents a Sp phosphorothioate;
m represents a 2′-OMe modified nucleoside; and
n001R is

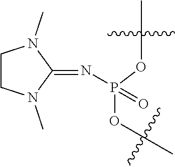 [SMILES: *OP(=O)(N=C1N(C)CCN1C)O*]

wherein the phosphorus is of the Rp configuration.

304. A composition, wherein a level of all oligonucleotides in the composition are oligonucleotides each independently having the structure of:
fC*SfA* SfGn001RfU*SfU*SfUn001RfC*SfC*SmUfU*SmA*SfG*SmUfA*SfA*SfC*SfUn001RfA*SfC*SfA (SEQ ID NO: 179), or a pharmaceutically acceptable salt thereof, wherein:
f represents a 2′-F modified nucleoside;
*S represents a Sp phosphorothioate;
m represents a 2′-OMe modified nucleoside; and
n001R is

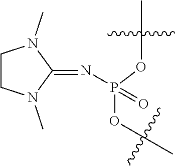 [SMILES: *OP(=O)(N=C1N(C)CCN1C)O*]

wherein the phosphorus is of the Rp configuration.

305. A composition, wherein a level of all oligonucleotides in the composition that have the base sequence of CAGUUUCCUUAGUAACCACA (SEQ ID NO: 422) are oligonucleotides each independently having the structure of:
fC*SfA* SfGn001RfU**SfU*SfUn001RfC*SfC*SmUfU*SmA*SfG*SmUfA*SfA*SfC*SfUn001RfA*SfC*SfA (SEQ ID NO: 179), or a pharmaceutically acceptable salt thereof, wherein:
f represents a 2′-F modified nucleoside;
*S represents a Sp phosphorothioate;
m represents a 2′-OMe modified nucleoside; and
n001R is

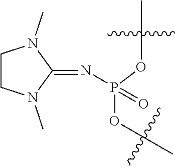 [SMILES: *OP(=O)(N=C1N(C)CCN1C)O*]

wherein the phosphorus is of the Rp configuration.

306. A composition, wherein a level of all oligonucleotides in the composition are oligonucleotides each independently having the structure of:
fA*SfG*SfUn001RfU*SfU*SfCn001RmCfU*SfU*SmA*SfG*SmUmA*SfA*SfC*SfC*SfAn001RfC*SfA*SfG (SEQ ID NO: 192), or a pharmaceutically acceptable salt thereof, wherein:
f represents a 2′-F modified nucleoside;
*S represents a Sp phosphorothioate;
m represents a 2′-OMe modified nucleoside; and
n001R is

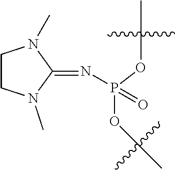 [SMILES: *OP(=O)(N=C1N(C)CCN1C)O*]

wherein the phosphorus is of the Rp configuration.

307. A composition, wherein a level of all oligonucleotides in the composition that have the base sequence of AGUUUCCUUAGUAACCACAG (SEQ ID NO: 392) are oligonucleotides each independently having the structure of:
fA* SfG*SfUn001RfU*SfU*SfCn001RmCfU* SfU*SmA*SfG*SmUmA* SfA* SfC*SfC*SfAn001RfC*SfA*SfU (SEQ ID NO: 192), or a pharmaceutically acceptable salt thereof, wherein:
f represents a 2′-F modified nucleoside;
*S represents a Sp phosphorothioate;
m represents a 2′-OMe modified nucleoside; and
n001R

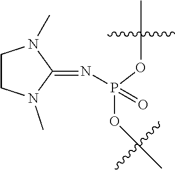 [SMILES: *OP(=O)(N=C1N(C)CCN1C)O*]

is wherein the phosphorus is of the Rp configuration.

308. A composition, wherein a level of all oligonucleotides in the composition are oligonucleotides each independently having the structure of:
fU*SfU*SfCn001RfC*SfU*SfUn001RmAfG*SfU*SmA*SfA*SmCmC*SfA*SfC*SfA*SfGn001RfG*SfU*SfU (SEQ ID NO: 193), or a pharmaceutically acceptable salt thereof, wherein:
f represents a 2′-F modified nucleoside;
*S represents a Sp phosphorothioate;
m represents a 2′-OMe modified nucleoside; and
n001R is

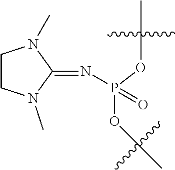 [SMILES: *OP(=O)(N=C1N(C)CCN1C)O*]

wherein the phosphorus is of the Rp configuration.

309. A composition, wherein a level of all oligonucleotides in the composition that have the base sequence of UUCCUUAGUAACCACAGGUU (SEQ ID NO: 423) are oligonucleotides each independently having the structure of:
fU*SfU*SfCn001RfC*SfU*SfUn001RmAfG*SfU*SmA*SfA*SmCmC*SfA*SfC*SfA*SfGn001RfG*SfU*SfU (SEQ ID NO: 193), or a pharmaceutically acceptable salt thereof, wherein:
f represents a 2′-F modified nucleoside;
*S represents a Sp phosphorothioate;
m represents a 2′-OMe modified nucleoside; and
n001R is

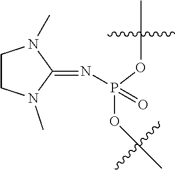 [SMILES: *OP(=O)(N=C1N(C)CCN1C)O*]

wherein the phosphorus is of the Rp configuration.

310. The composition of any one of embodiments 282-309, wherein the composition is a liquid composition, wherein the oligonucleotides are one or more salts dissolved in the composition. 311. The composition of any one of embodiments 282-310, wherein the oligonucleotides are each independently a pharmaceutically acceptable salt. 312. The composition of any one of embodiments 282-311, wherein the oligonucleotides are each independently a pharmaceutically acceptable salt independently selected from a sodium salt, a potassium salt and an ammonium (e.g., having the structure of [N(R)₄]⁺ (e.g., wherein each R is independently —H or optionally substituted C₁₋₆ alkyl)) salt.

313. The composition of any one of embodiments 282-312, wherein the oligonucleotides are each independently a sodium salt.

314. The composition of any one of embodiments 282-313, wherein the level is about 1%-100% (e.g., about 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10/, 15%, 20%, 25%, 30%, 35%, 40%, 45%, 50%, 55%, 60%, 65%, 70%, 75%, 80%, 85%, 90%, 95%, or 99%).

315. The composition of any one of embodiments 282-313, wherein the level is about 50% or more.

316. The composition of any one of embodiments 282-313, wherein the level is about 60% or more.

317. The composition of any one of embodiments 282-313, wherein the level is about 70% or more.

318. The composition of any one of embodiments 282-313, wherein the level is about 80% or more.

319. The composition of any one of embodiments 282-313, wherein the level is about 85% or more.

320. The composition of any one of embodiments 282-313, wherein the level is about 90% or more.

321. The composition of any one of embodiments 282-313, wherein the level is about 95% or more.

322. An oligonucleotide having the structure of:
fG* SfU* SfAn001RfC*SfC*SfUn001RfC*SfC*SmAfA*SmC*SfA*SmUfC*SfA*SfA*SfGn001RfG*SfA*SfA (SEQ ID NO: 172), or a pharmaceutically acceptable salt thereof, wherein:
f represents a 2′-F modified nucleoside:
S represents a Sp phosphorothioate;
m represents a 2′-OMe modified nucleoside; and
n001R is

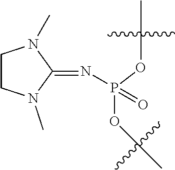 [SMILES: *OP(=O)(N=C1N(C)CCN1C)O*]

wherein the phosphorus is of the Rp configuration.

323. An oligonucleotide having the structure of:
fG*SfG*SfGn001RfC*SfA*SfUn001RfU*SfU*SmCfU*SmA*SfG*SmUfU*SfU*SfG*SfGn001RfA*SfG*SfA (SEQ ID NO: 175), or a pharmaceutically acceptable salt thereof, wherein:
f represents a 2′-F modified nucleoside;
*S represents a Sp phosphorothioate;
m represents a 2′-OMe modified nucleoside; and
n001R is

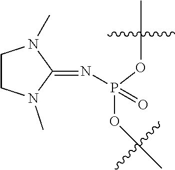 [SMILES: *OP(=O)(N=C1N(C)CCN1C)O*]

wherein the phosphorus is of the Rp configuration.

324. An oligonucleotide having the structure of:
fG*SfG*SfCn001RfA*SfU*SfUn001RmUfC*SfU*SmA*SfG*SmUmU*SfU*SfG*SfG*SfAn001RfG*SfA*SfU (SEQ ID NO: 188), or a pharmaceutically acceptable salt thereof, wherein:
f represents a 2′-F modified nucleoside;
*S represents a Sp phosphorothioate;
m represents a 2′-OMe modified nucleoside; and
n001R is

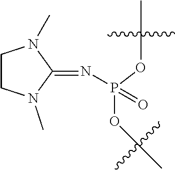 [SMILES: *OP(=O)(N=C1N(C)CCN1C)O*]

wherein the phosphorus is of the Rp configuration.

325. An oligonucleotide having the structure of:
fU*SfG*SfGn001RfC*SfA*SfGn001RfU*SfU*SmUfC*SmC*SfU*SmUfA*SfG* SfU*SfAn001 RfA*SfC*SfC (SEQ ID NO: 178), or a pharmaceutically acceptable salt thereof, wherein:
f represents a 2′-F modified nucleoside;
*S represents a Sp phosphorothioate;
m represents a 2′-OMe modified nucleoside; and
n001R is

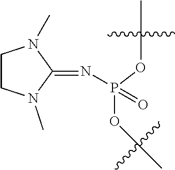 [SMILES: *OP(=O)(N=C1N(C)CCN1C)O*]

wherein the phosphorus is of the Rp configuration.

326. An oligonucleotide having the structure of:
fU*SfG*SfGn001RfC*SfA*SfGn001RmUfU*SfU*SmC*SfC*SmUmU*SfA*SfG*SfU*SfAn001RfA*SfC*SfC (SEQ ID NO: 190), or a pharmaceutically acceptable salt thereof, wherein:
f represents a 2′-F modified nucleoside;
*S represents a Sp phosphorothioate;
m represents a 2′-OMe modified nucleoside; and
n001R

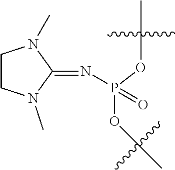 [SMILES: *OP(=O)(N=C1N(C)CCN1C)O*]

is wherein the phosphorus is of the Rp configuration.

327. An oligonucleotide having the structure of:
fG*SfG*SfUn001RfA*SfA*SfGn001RfU*SfU*SmCfU*SmG*SfU*SmCfC*SfA*SfA*SfGn001RfC*SfC*SfC (SEQ ID NO: 171), or a pharmaceutically acceptable salt thereof, wherein:
f represents a 2′-F modified nucleoside;
*S represents a Sp phosphorothioate;
m represents a 2′-OMe modified nucleoside; and
n001R is

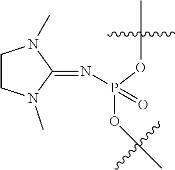 [SMILES: *OP(=O)(N=C1N(C)CCN1C)O*]

wherein the phosphorus is of the Rp configuration.

328. An oligonucleotide having the structure of:
fG*SfG*SfUn001RfA*SfA*SfUn001RmUfU*Sf*SmUmC*SfC*SfA*SfGn001RfC*SfC*SfC (SEQ ID NO: 183), or a pharmaceutically acceptable salt thereof, wherein:
f represents a 2′-F modified nucleoside;
*S represents a Sp phosphorothioate;
m represents a 2′-OMe modified nucleoside; and
n001R is

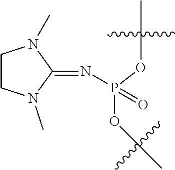 [SMILES: *OP(=O)(N=C1N(C)CCN1C)O*]

wherein the phosphorus is of the Rp configuration.

329. An oligonucleotide having the structure of:
fC*SfA*SfAn001RfC*SfA*SfUn0001RfC*SfA*SmAfG*SmG*SfA*SmAfG*SfA*SfU*SfGn001RfG*SfC*SfA (SEQ ID NO: 173), or a pharmaceutically acceptable salt thereof, wherein:
f represents a 2′-F modified nucleoside;
*S represents a Sp phosphorothioate;
m represents a 2′-OMe modified nucleoside; and
n001R is

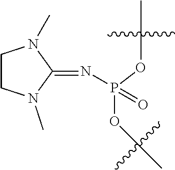 [SMILES: *OP(=O)(N=C1N(C)CCN1C)O*]

wherein the phosphorus is of the Rp configuration.

330. An oligonucleotide having the structure of:
fA*SfU*SfGn001RfG*SfC*SfAn001RfU*SfU*SmUfC*SmU*SfA*SmGfU*SfU*SfU*SfGn001RfG*SfA*SfG (SEQ ID NO: 174), or a pharmaceutically acceptable salt thereof, wherein:
f represents a 2-F modified nucleoside;
*S represents a Sp phosphorothioate;
m represents a 2′-OMe modified nucleoside; and
n001R is

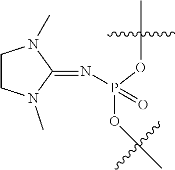 [SMILES: *OP(=O)(N=C1N(C)CCN1C)O*]

wherein the phosphorus is of the Rp configuration.

331. An oligonucleotide having the structure of:
fA*SfU*SfGn001RfG*SfC*SfAn001RmUfU*SfU*SmC*SfU*SmAmG*SfU*SfU*SfU*SfGn001RfG*SfA*SfG (SEQ ID NO: 186), or a pharmaceutically acceptable salt thereof, wherein:
f represents a 2′-F modified nucleoside;
*S represents a Sp phosphorothioate;
m represents a 2′-OMe modified nucleoside; and
n001R is

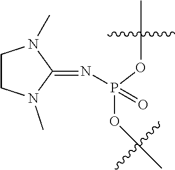 [SMILES: *OP(=O)(N=C1N(C)CCN1C)O*]

wherein the phosphorus is of the Rp configuration.

332. An oligonucleotide having the structure of:
fG*SfC*SfAn001RfU*SfU*SfUn001RfC*SfU*SmAfG*SmU*SfU*SmUfG*SfG*SfA*SfGn001 RfA*SfU*SfG (SEQ ID NO: 177), or a pharmaceutically acceptable salt thereof, wherein:
f represents a 2′-F modified nucleoside;
*S represents a Sp phosphorothioate;
m represents a 2′-OMe modified nucleoside; and
n001R is

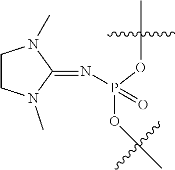 [SMILES: *OP(=O)(N=C1N(C)CCN1C)O*]

wherein the phosphorus is of the Rp configuration.

333. An oligonucleotide having the structure of:
fU*SfA*SfGn001RfU*SfU*SfUn001RfC*SfC*SmUfU*SmA*SfG*SmUfA*SfA*SfC*Sfn001RfA*SfC*SfA (SEQ ID NO: 179), or a pharmaceutically acceptable salt thereof, wherein:
f represents a 2′-F modified nucleoside;
*S represents a Sp phosphorothioate;
m represents a 2′-OMe modified nucleoside; and
n001R is

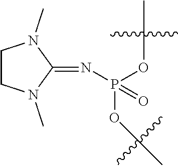 [SMILES: *OP(=O)(N=C1N(C)CCN1C)O*]

wherein the phosphorus is of the Rp configuration.

334. An oligonucleotide having the structure of:
fA* SfG*SfUn001RfU*SfU*SfCn001RmCfU*SfU*SmA*SfG*SmUmA*SfA*SfC*SfC*SfAn001RfC*SfA*SfG (SEQ ID NO: 192), or a pharmaceutically acceptable salt thereof, wherein:
f represents a 2′-F modified nucleoside;
*S represents a Sp phosphorothioate;
m represents a 2′-OMe modified nucleoside; and
n001R is

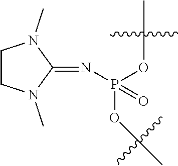 [SMILES: *OP(=O)(N=C1N(C)CCN1C)O*]

f represents a 2′-F modified nucleoside;
*S represents a Sp phosphorothioate;
m represents a 2′-OMe modified nucleoside; and
wherein the phosphorus is of the Rp configuration.

335. An oligonucleotide having the structure of:
fU*SfU*SfCn001RfC*SfU*SfUn001RmAfG*SfU*SmA*SfA*SmCmC*SfA*SfC*SfA*SfGn001RfG*SfU*SfU (SEQ ID NO: 193), or a pharmaceutically acceptable salt thereof, wherein:
n001R is

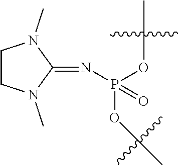 [SMILES: *OP(=O)(N=C1N(C)CCN1C)O*]

wherein the phosphorus is of the Rp configuration.

336. The oligonucleotide of any one of embodiments 322-335, wherein the oligonucleotide is a pharmaceutically acceptable salt.

337. The oligonucleotide of any one of embodiments 322-336, wherein the oligonucleotide is a pharmaceutically acceptable salt selected from a sodium salt, a potassium salt and an ammonium (e.g., having the structure of [N(R)₄]⁺ (e.g., wherein each R is independently —H or optionally substituted C₁₋₆ alkyl)) salt.

338. The oligonucleotide of any one of embodiments 322-337, wherein the oligonucleotide is a sodium salt.

339. The oligonucleotide of any one of embodiments 322-338, wherein the oligonucleotide has a diastereomeric purity of about 1%-100% (e.g., about 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 99%).

340. The oligonucleotide of any one of embodiments 322-339, wherein the diastereomeric purity is about 50% or more.

341. The oligonucleotide of any one of embodiments 322-339, wherein the diastereomeric purity is about 60% or more.

342. The oligonucleotide of any one of embodiments 322-339, wherein the diastereomeric purity is about 70% or more.

343. The oligonucleotide of any one of embodiments 322-339, wherein the diastereomeric purity is about 80% or more.

344. The oligonucleotide of any one of embodiments 322-339, wherein the diastereomeric purity is about 85% or more.

345. The oligonucleotide of any one of embodiments 322-339, wherein the diastereomeric purity is about 90% or more.

346. The oligonucleotide of any one of embodiments 322-339, wherein the diastereomeric purity is about 95% or more.

347. A pharmaceutical composition, comprising an effective amount of an oligonucleotide of any one of embodiments 322-346, and a pharmaceutically acceptable carrier.

348. A method for altering splicing of a target transcript, comprising administering an oligonucleotide or oligonucleotide composition of any one of the preceding embodiments.

349. The method of embodiment 348, wherein the splicing of the target transcript is altered relative to absence of the composition.

350. The method of any one of the preceding embodiments, wherein the alteration is that one or more exon is skipped at an increased level relative to absence of the composition.

351. The method of any one of the preceding embodiments, wherein the target transcript is pre-mRNA of dystrophin.

352. The method of any one of the preceding embodiments, wherein exon 45 of dystrophin is skipped at an increased level relative to absence of the composition.

353. The method of any one of the preceding embodiments, wherein exon 51 of dystrophin is skipped at an increased level relative to absence of the composition.

354. The method of any one of embodiments 348-351, wherein exon 53 of dystrophin is skipped at an increased level relative to absence of the composition.

355. The method of any one of the preceding embodiments, wherein a protein encoded by the mRNA with the exon skipped provides one or more functions better than a protein encoded by the corresponding mRNA without the exon skipping.

356. A method for treating muscular dystrophy, Duchenne (Duchenne's) muscular dystrophy (DMD), or Becker (Becker's) muscular dystrophy (BMD), comprising administering to a subject susceptible thereto or suffering therefrom a composition of any one of the preceding embodiments.

357. A method for treating muscular dystrophy, Duchenne (Duchenne's) muscular dystrophy (DMD), or Becker (Becker's) muscular dystrophy (BMD), comprising administering to a subject susceptible thereto or suffering therefrom a composition comprising any oligonucleotide disclosed herein.

358. A method for treating muscular dystrophy, Duchenne (Duchenne's) muscular dystrophy (DMD), or Becker (Becker's) muscular dystrophy (BMD), comprising (a) administering to a subject susceptible thereto or suffering therefrom a composition comprising any oligonucleotide disclosed herein, and (b) administering to the subject additional treatment which is capable of preventing, treating, ameliorating or slowing the progress of muscular dystrophy, Duchenne (Duchenne's) muscular dystrophy (DMD), or Becker (Becker's) muscular dystrophy (BMD).

359. The method of embodiment 358, wherein the additional treatment is a second oligonucleotide.

360. The composition of any of the preceding embodiments, wherein the transcript splicing system comprises a myoblast or myotubule.

361. The composition of any of the preceding embodiments, wherein the transcript splicing system comprises a myoblast cell.

362. The composition of any of the preceding embodiments, wherein the transcript splicing system comprises a myoblast cell, which is contacted with the composition after 0, 4 or 7 days of pre-differentiation.

363. A composition comprising a combination comprising: (a) a first composition of any of the preceding embodiments; (b) a second composition of any of the preceding embodiments, and, optionally (c) a third composition of any of the preceding embodiments, wherein the first, second and third compositions are different.

364. A method for preparing an oligonucleotide or an oligonucleotide composition thereof, comprising providing a compound having the structure of:

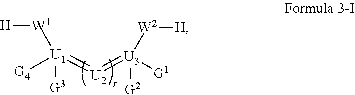

or a salt thereof.

365. A method for preparing an oligonucleotide or an oligonucleotide composition thereof, comprising providing a compound having the structure of:

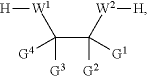

or a salt thereof.

366. A method for preparing an oligonucleotide or an oligonucleotide composition thereof, comprising providing a compound having the structure of

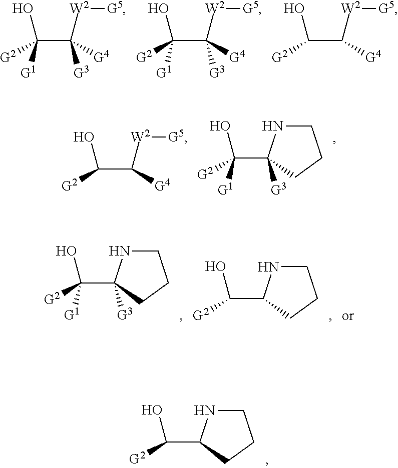

or a salt thereof.

367. The method of any one of embodiments 364-366, wherein the compound is stereochemically pure.

368. The method of any one of embodiments 364-367, wherein the compound is a compound of Tables CA-1, CA-2, CA-3, CA-4, CA-5, CA-6, CA-7, CA-8, CA-9, CA-10, CA-11, or CA-12, or a related diastereomer or enantiomer thereof.

369. The method of any one of embodiments 364-367, wherein the compound is a compound of Table CA-2 or a related diastereomer or enantiomer thereof.

370. The method of any one of embodiments 364-367, wherein the compound is a compound of Table CA-3 or a related diastereomer or enantiomer thereof.

371. The method of any one of embodiments 364-367, wherein the compound is a compound of Table CA-4 or a related diastereomer or enantiomer thereof.

372. The method of any one of embodiments 364-367, wherein the compound is a compound of Table CA-5 or a related diastereomer or enantiomer thereof.

373. The method of any one of embodiments 364-367, wherein the compound is a compound of Table CA-6 or a related diastereomer or enantiomer thereof.

374. The method of any one of embodiments 364-367, wherein the compound is a compound of Table CA-7 or a related diastereomer or enantiomer thereof.

375. The method of any one of embodiments 364-367, wherein the compound is a compound of Table CA-8 or a related diastereomer or enantiomer thereof.

376. The method of any one of embodiments 364-367, wherein the compound is a compound of Table CA-9 or a related diastereomer or enantiomer thereof.

377. The method of any one of embodiments 364-367, wherein the compound is a compound of Table CA-10 or a related diastereomer or enantiomer thereof.

378. The method of any one of embodiments 364-367, wherein the compound is a compound of Table CA-11 or a related diastereomer or enantiomer thereof.

379. The method of any one of embodiments 364-367, wherein the compound is a compound of Table CA-12 or a related diastereomer or enantiomer thereof.

380. A method for preparing an oligonucleotide or an oligonucleotide composition thereof, comprising providing a phosphoramidite compound comprising a chiral auxiliary moiety having the structure of

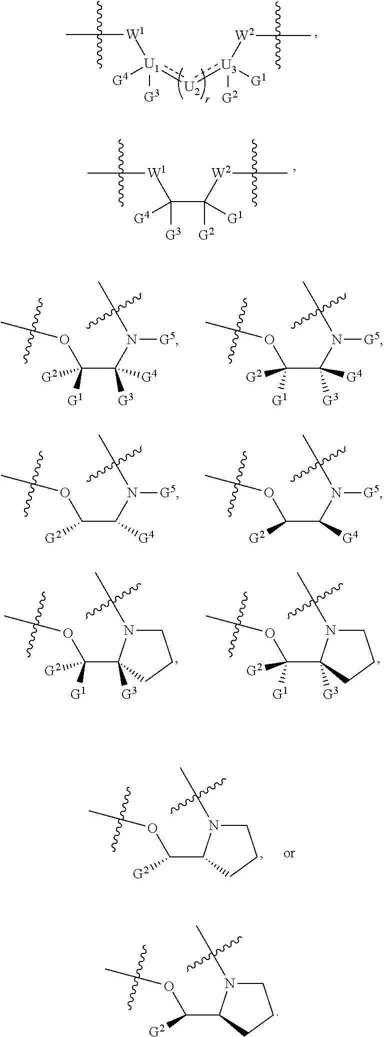

381. A method for preparing an oligonucleotide or an oligonucleotide composition thereof, comprising providing a phosphoramidite compound having the structure of:

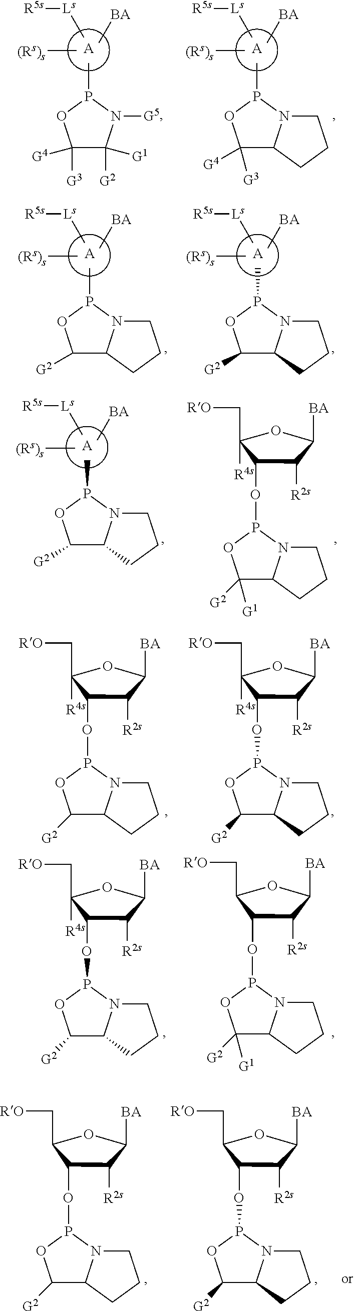

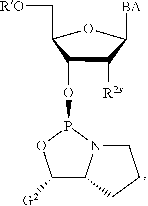

or a salt thereof.

382. The method of any one of embodiments 364-381, wherein W¹ is —NG⁵-.

383. The method of any one of embodiments 364-382, wherein G⁵ and one of G³ and G⁴ are taken together to form an optionally substituted 3-8 membered saturated ring having 0-3 heteroatoms in addition to the nitrogen of —NG⁵-.

384. The method of any one of embodiments 364-382, wherein G⁵ and one of G³ and G⁴ are taken together to form an optionally substituted 5-membered saturated ring having no heteroatoms in addition to the nitrogen of —NG⁵-.

385. The method of any one of embodiments 364-384, wherein W² is —O—.

386. The method of any one of embodiments 364-385, wherein G² comprises an electron-withdrawing group.

387. The method of any one of embodiments 364-385, wherein G² is methyl substituted with one or more electron-withdrawing groups.

388. The method of any one of embodiments 386-387, wherein an electron-withdrawing group is —CN, —NO₂, halogen, —C(O)R¹, —C(O)OR′, —C(O)N(R′)₂, —S(O)R¹, —S(O)₂R¹, —P(W)(R¹)₂, —P(O)(R¹)₂, —P(O)(OR′)₂, or —P(S)X(R¹)₂, or aryl or heteroaryl substituted with one or more of —CN, —NO₂, halogen, —C(O)R¹, —C(O)OR′, —C(O)N(R′)₂, —S(O)R¹, —S(O)₂R¹, —P(W)(R¹)₂, —P(O)(R¹)₂, —P(O)(OR′)₂, or —P(S)(R¹)₂.

389. The method of any one of embodiments 386-387, wherein an electron-withdrawing group is —CN, —NO₂, halogen, —C(O)R¹, —C(O)OR′, —C(O)N(R′)₂, —S(O)R¹, —S(O)₂R¹, —P(W)(R¹)₂, —P(O)(R¹)₂, —P(O)(OR′)₂, or —P(S)(R¹)₂, or phenyl substituted with one or more of —CN, —NO₂, halogen, —C(O)R¹, —C(O)OR′, —C(O)N(R′)₂, —S(O)R¹, —S(O)₂R¹, —P(W)(R¹)₂, —P(O)(R′)₂, —P(O)(OR′)₂, or —P(S)(R¹)₂.

390. The method of any one of embodiments 386-387, wherein an electron-withdrawing group is —CN, —NO₂, halogen, —C(O)R¹, —C(O)OR′, —C(O)N(R′)₂, —S(O)R¹, —S(O)₂R¹, —P(W)(R¹)₂, —P(O)(R¹)₂, —P(O)(OR′)₂, or —P(S)(R¹)₂.

391. The method of any one of embodiments 364-386, wherein G² is -L′-L″-R′, wherein L′ is —C(R)₂— or optionally substituted —CH₂—, and L″ is a covalent bond, —P(O)(R′)—, —P(O)(R′)O—, —P(O)(OR′)—, —P(O)(OR′)O—, —P(O)[N(R′)]—, —P(O)[N(R′)]O—, —P(O)[N(R′)][N(R′)]—, —P(S)(R′)—, —S(O)₂—, —S(O)₂—, —S(O)₂O—, —S(O)—, —C(O)—, or —C(O)N(R′)—.

392. The method of any one of embodiments 364-386, wherein G² is -L′-L″-R′, wherein L′ is —C(R)₂—or optionally substituted —CH₂—, and L″ is —P(O)(R′)—, —P(O)(R′)O—, —P(O)(OR′)—, —P(O)(OR′)O—, —P(O)[N(R′)]—, —P(O)[N(R′)]O—, —P(O)[N(R′)][N(R′)]—, —P(S)(R′)—, —S(O)₂—, —S(O)₂—, —S(O)₂O—, —S(O)—, —C(O)—, or —C(O)N(R′)—.

393. The method of any one of embodiments 364-392, wherein G² is -L′-S(O)₂R′.

394. The method of embodiment 393, wherein R′ is optionally substituted C₁₋₆ aliphatic.

395. The method of embodiment 393, wherein R′ is optionally substituted C₁₋₆ alkyl.

396. The method of embodiment 393, wherein R′ is methyl, isopropyl or t-butyl.

397. The method of embodiment 393, wherein R′ is optionally substituted phenyl.

398. The method of embodiment 393, wherein R′ is phenyl.

399. The method of embodiment 393, wherein R′ is substituted phenyl.

400. The method of any one of embodiments 364-392, wherein G² is -L′-P(O)(R′)₂.

401. The method of embodiment 400, wherein one R′ is optionally substituted C₁₋₆ aliphatic.

402. The method of embodiment 400, wherein one R′ is optionally substituted C₁₋₆ alkyl.

403. The method of embodiment 400, wherein one R′ is optionally substituted phenyl.

404. The method of embodiment 400, wherein one R′ is phenyl.

405. The method of embodiment 400, wherein one R′ is substituted phenyl.

406. The method of any one of embodiments 401-405, wherein the other R′ is optionally substituted C₁₋₆ aliphatic.

407. The method of any one of embodiments 401-405, wherein the other R′ is optionally substituted C₁₋₆ alkyl.

408. The method of any one of embodiments 401-405, wherein the other R′ is optionally substituted phenyl.

409. The method of any one of embodiments 401-405, wherein the other R′ is phenyl.

410. The method of any one of embodiments 401-405, wherein the other R′ is substituted phenyl.

411. The method of any one of embodiments 391-410, wherein L′ is —C(R′)₂—.

412. The method of any one of embodiments 391-410, wherein L′ is optionally substituted —CH₂—.

413. The method of any one of embodiments 391-410, wherein L′ is —CH₂—.

414. The method of any one of embodiments 364-413, comprising providing one or more additional compounds, wherein each compound is independently a compound of any one of embodiments 364-413.

415. The method of embodiment 414, wherein an additional compound has a different structure than the compound.

416. The method of embodiment 414, wherein in an additional compound, G² is -L′-Si(R)₃, wherein each R is independently not —H.

417. The method of embodiment 414, wherein in an additional compound, G² is —CH₂SiCH₃Ph₂.

418. The method of any one of embodiments 364-417, comprising one or more cycles, each of which independently comprises or consisting of:
1) deblocking;
2) coupling;
3) optionally a first capping;
4) modifying; and
5) optionally a second capping.

419. A method for preparing an oligonucleotide or a composition thereof, comprising one or more cycles, each of which independently comprises or consisting of:
1) deblocking;
2) coupling;
3) optionally a first capping;
4) modifying; and
5) optionally a second capping.

420. The method of any one of embodiments 418-419, wherein at least one cycle comprises or consists of 1) to 5).

421. The method of any one of embodiments 418-420, wherein the steps are performed sequentially from 1) to 5).

422. The method of any one of embodiments 418-421, wherein the cycles are performed until a desired length of an oligonucleotide is achieved.

423. The method of any one of embodiments 418-422, wherein deblocking removes a protection group on 5′-OH and provides a free 5′-OH.

424. The method of embodiment 423, wherein the protection group is R′—C(O)—.

425. The method of embodiment 423, wherein the protection group is DMTr.

426. The method of any one of embodiments 423-425, comprising contacting the oligonucleotides to be de-blocked with an acid.

427. The method of any one of embodiments 364-426, comprising a coupling that comprises: 1) providing a phosphoramidite; and 2) reacting the phosphoramidite with an oligonucleotide, wherein a P—O bond is formed between the phosphorus of the phosphoramidite and the 5′-OH of the oligonucleotide.

428. The method of any one of embodiments 364-427, comprising a coupling that comprises: 1) providing a phosphoramidite; and 2) reacting the phosphoramidite with an oligonucleotide, wherein a P—O bond is formed between the phosphorus of the phosphoramidite and the 5′-OH of the oligonucleotide, wherein the phosphoramidite is a compound of any one of embodiments 380-413.

429. The method of any one of embodiments 364-428, comprising a coupling that comprises: 1) providing a phosphoramidite; and 2) reacting the phosphoramidite with an oligonucleotide, wherein a P—O bond is formed between the phosphorus of the phosphoramidite and the 5′-OH of the oligonucleotide, wherein the phosphoramidite is a compound of any one of embodiments 380-385, wherein G² is -L′-Si(R)₃, wherein each R is independently not —H.

430. The method of embodiment 429, wherein G² is —CH₂SiCH₃Ph₂.

431. The method of any one of embodiments 428-430, wherein the coupling forms an internucleotidic linkage with a stereoselectivity of 85%, 90%, 91%, 92%, 93%, 94%, 95%, 96%, 97%, 98%, 99% or more.

432. The method of embodiment 431, wherein the internucleotidic linkage formed is an internucleotidic linkage of formula I or a salt form thereof.

433. The method of embodiment 432, wherein —X-L-R¹ is

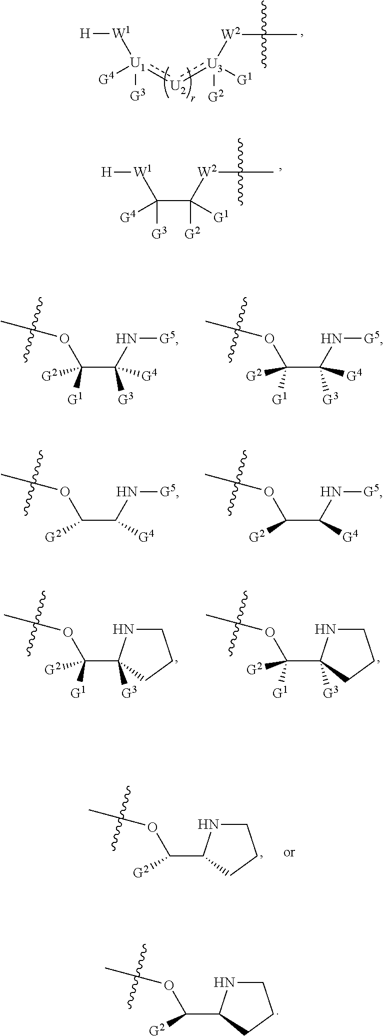

434. The method of embodiment 432 or 433, wherein P^{L} is P.

435. The method of any one of embodiments 364-434, comprising a coupling that comprises: 1) providing a phosphoramidite; and 2) reacting the phosphoramidite with an oligonucleotide, wherein a P—O bond is formed between the phosphorus of the phosphoramidite and the 5′-OH of the oligonucleotide, wherein the phosphoramidite is a standard phosphoramidite for oligonucleotide synthesis wherein the phosphorus atom is bonded to a protected nucleoside, —N(i-Pr)₂, and 2-cyanoethyl.

436. The method of any one of embodiments 364-435, comprising a first capping comprises: 1) providing an acylating reagent, and 2) contacting an oligonucleotide with the acylating reagent, wherein the first capping caps an amino group of an internucleotidic linkage.

437. The method of any one of embodiments 364-436, comprising a first capping which forms an internucleotidic linkage of formula I or a salt form thereof, wherein —X-L-R¹ is

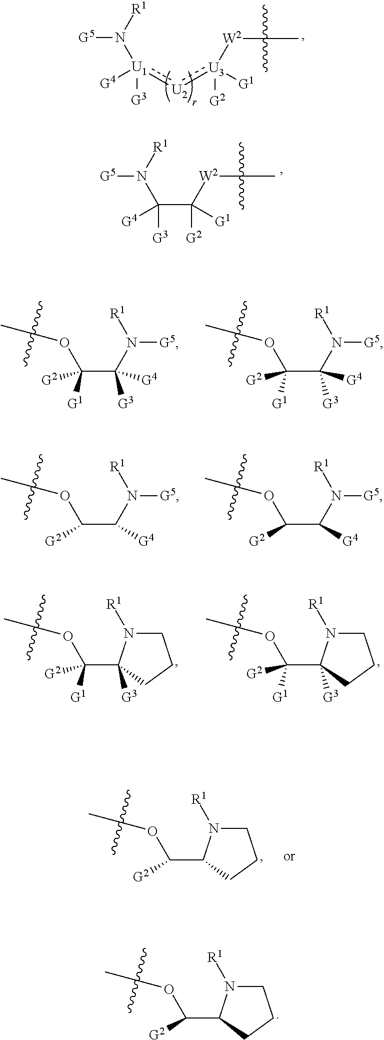

438. The method of embodiment 437, wherein P^{L} is P and R¹ is —C(O)R.

439. The method of any one of embodiments 364-438, wherein a first capping is performed after each coupling of embodiment 431.

440. The method of any one of embodiments 364-439, comprising a modifying step which is or comprises sulfurization.

441. The method of embodiment 440, wherein the sulfurization installs ═S on a linkage phosphorus.

442. The method of embodiment 440 or 441, wherein the sulfurization forms an internucleotidic linkage of formula I or a salt form thereof, wherein P^{L} is P(═S).

443. The method of embodiment 442, wherein —X-L-R¹ is

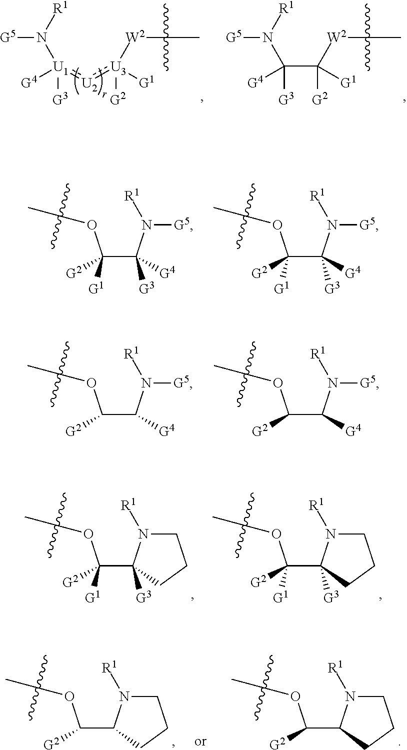

444. the method of embodiment 443, wherein R¹ is —C(O)R.

445. The method of any one of embodiments 364-444, comprising a modifying step which is or comprises oxidation.

446. The method of embodiment 440, wherein the sulfurization installs ═O on a linkage phosphorus.

447. The method of any one of embodiments 364-446, comprising a modifying step which installs ═N-L-R⁵ on a linkage phosphorus.

448. The method of any one of embodiments 364-446, comprising a modifying step which converts a linkage phosphorus into

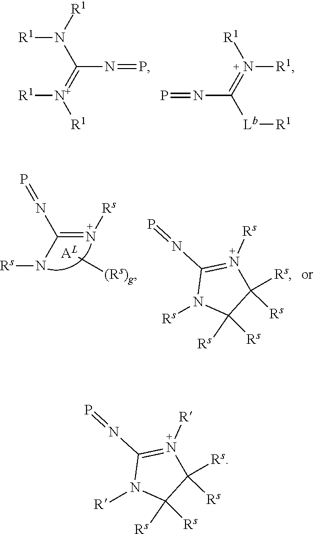

449. The method of any one of embodiments 364448, comprising a modifying step which comprises contact the oligonucleotide with an azido imidazolinium salt.

450. The method of any one of embodiments 364-448, comprising a modifying step which comprises contact the oligonucleotide with a compound comprising

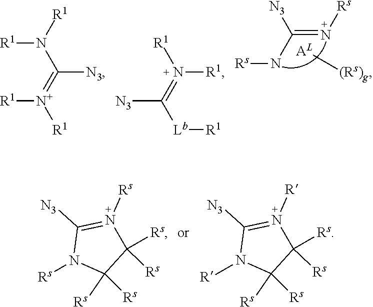

451. The method of any one of embodiments 364-448, comprising a modifying step which comprises contact the oligonucleotide with a compound having the structure of

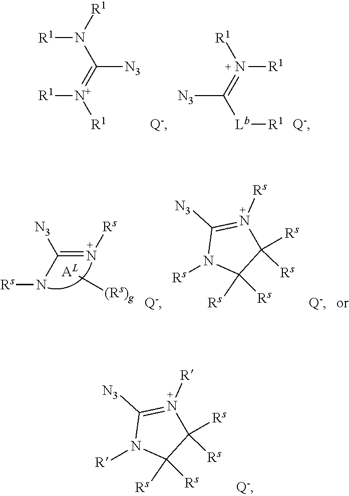

wherein Q⁻ is an anion.

452. The method of embodiment 451, wherein Q⁻ is F⁻, Cl⁻, Br⁻, BF₄⁻, PF₆⁻, TfO⁻, Tf₂N⁻, AsF₆⁻, ClO₄⁻, or SbF₆⁻.

453. The method of embodiment 452, wherein Q⁻ is PF₆⁻.

454. The method of any one of embodiments 364-454, wherein a modifying step forms an internucleotidic linkage of formula I or a salt form thereof, wherein P^{L} is P(═N-L-R⁵).

455. The method of any one of embodiments 364-454, wherein a modifying step forms an internucleotidic linkage of formula III or a salt form thereof.

456. The method of embodiment 454 or 455, wherein —X-L-R¹ is

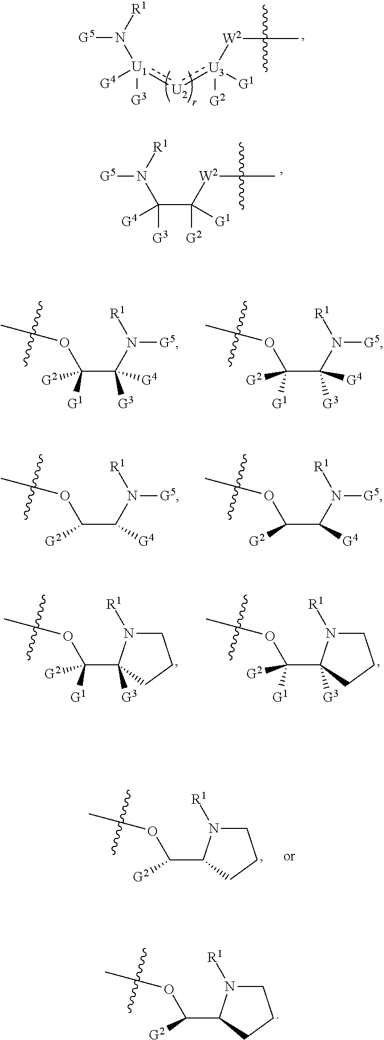

457. The method of embodiment 456, wherein R¹ is —C(O)R.

458. The method of any one of embodiments 364-457, comprising a second capping which caps free 5′-OH.

459. The method of any one of embodiments 364-458, comprising a second capping which caps free 5′-OH, wherein a second capping is performed in each cycle.

460. The method of any one of embodiments 364-458, comprising a second capping which caps free 5′-OH, wherein a second capping is performed in each cycle that is followed by another cycle.

461. The method of any one of embodiments 458-460, wherein a 5′-OH is capped as —OAc.

462. The method of any one of embodiments 364-461, wherein the oligonucleotide is attached to a solid support.

463. The method of embodiment 462, wherein the solid support is CPG.

464. The method of any one of embodiments 462-463, comprising a contact in which the oligonucleotide is contacted with a base.

465. The method of embodiment 464, wherein the contact is performed substantially absent of water.

466. The method of embodiment 464 or 465, wherein the contact is after the oligonucleotide length is achieved before deprotection and cleavage of oligonucleotide.

467. The method of any one of embodiments 464-466, wherein the base is an amine base having the structure of NR₃.

468. The method of embodiment 467, wherein the base is triethylamine.

469. The method of embodiment 467, wherein the base is N. N-diethylamine.

470. The method of any one of embodiments 464-469, wherein the contact removes a chiral auxiliary.

471. The method of any one of embodiments 464-470, wherein the contact removes a —X-L-R¹ group.

472. The method of embodiment 471, wherein —X-L-R¹ is

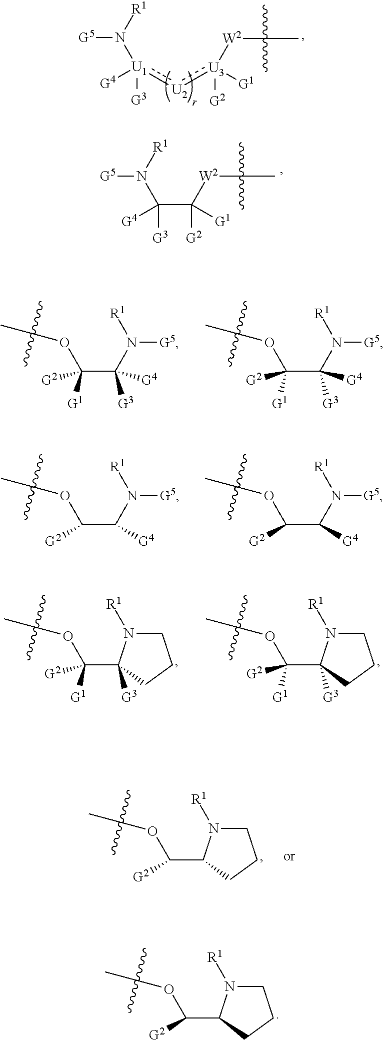

473. The method of any one of embodiments 464-472, wherein the contact forms an internucleotidic linkage of formula I-n-1, I-n-2, I-n-3, I-n-4, II, II-a-1, II-a-2, II-b-1, II-b-2, II-c-1, II-c-2, II-d-1, or II-d-2, wherein P^{L} is P(O).

474. The method of any one of embodiments 456-473, wherein G² comprises an electron-withdrawing group.

475. The method of any one of embodiments 456-474, wherein G² is methyl substituted with one or more electron-withdrawing groups.

476. The method of any one of embodiments 474-475, wherein an electron-withdrawing group is —CN, —NO₂, halogen, —C(O)R¹, —C(O)OR′, —C(O)N(R′)₂, —S(O)R¹, —S(O)₂R¹, —P(W)(R¹)₂, —P(O)(R¹)₂, —P(O)(OR′)₂, or —P(S)(R¹)₂, or aryl or heteroaryl substituted with one or more of —CN, —NO₂, halogen, —C(O)R¹, —C(O)OR′, —C(O)N(R′)₂, —S(O)R¹, —S(O)₂R¹, —P(W)(R¹)₂, —P(O)(R¹)₂, —P(O)(OR′)₂, or —P(S)(R¹)₂.

477. The method of any one of embodiments 474-475, wherein an electron-withdrawing group is —CN, —NO₂, halogen, —C(O)R¹, —C(O)OR′, —C(O)N(R′)₂, —S(O)R¹, —S(O)₂R¹, —P(W)(R¹)₂, —P(O)(R¹)₂, —P(O)(OR′)₂, or —P(S)(R¹)₂, or phenyl substituted with one or more of —CN, —NO₂, halogen, —C(O)R¹, —C(O)OR′, —C(O)N(R′)₂, —S(O)R¹, —S(O)₂R¹, —P(W)(R¹)₂, —P(O)(R¹)₂, —P(O)(OR′)₂, or —P(S)(R¹)₂.

478. The method of any one of embodiments 474-475, wherein an electron-withdrawing group is —CN, —NO₂, halogen, —C(O)R¹, —C(O)OR′, —C(O)N(R′)₂, —S(O)R¹, —S(O)₂R¹, —P(W)(R¹)₂, —P(O)(R¹)₂, —P(O)(OR′)₂, or —P(S)(R¹)₂.

479. The method of any one of embodiments 456-478, wherein G² is -L′-L″-R′, wherein L′ is —C(R)₂—or optionally substituted —CH₂—, and L″ is a covalent bond, —P(O)(R′)—, —P(O)(R′)O—, —P(O)(OR′)—, —P(O)(OR′)O—, —P(O)[N(R′)]—, —P(O)[N(R′)]O—, —P(O)[N(R′)][N(R′)]—, —P(S)(R′)—, —S(O)₂—, —S(O)₂—, —S(O)₂O—, —S(O)—, —C(O)—, or —C(O)N(R′)—.

480. The method of any one of embodiments 456-478, wherein G² is -L′-L″-R′, wherein L′ is —C(R)₂—or optionally substituted —CH₂—, and L″ is —P(O)(R′)—, —P(O)(R′)O—, —P(O)(OR′)—, —P(O)(OR′)O—, —P(O)[N(R′)]—, —P(O)[N(R′)]O—, —P(O)[N(R′)][N(R′)]—, —P(S)(R′)—, —S(O)₂—, —S(O)₂—, —S(O)₂O—, —S(O)—, —C(O)—, or —C(O)N(R′)—.

481. The method of any one of embodiments 456-480, wherein G² is -L′-S(O)₂R′.

482. The method of embodiment 481, wherein R′ is optionally substituted C₁₋₆ aliphatic.

483. The method of embodiment 481, wherein R′ is optionally substituted C₁₋₆ alkyl.

484. The method of embodiment 481, wherein R′ is methyl, isopropyl or t-butyl.

485. The method of embodiment 481, wherein R′ is optionally substituted phenyl.

486. The method of embodiment 481, wherein R′ is phenyl.

487. The method of embodiment 481, wherein R′ is substituted phenyl.

488. The method of any one of embodiments 456-480, wherein G² is -L′-P(O)(R′)₂.

489. The method of embodiment 488, wherein one R′ is optionally substituted C₁₋₆ aliphatic.

490. The method of embodiment 488, wherein one R′ is optionally substituted C₁₋₆ alkyl.

491. The method of embodiment 488, wherein one R′ is optionally substituted phenyl.

492. The method of embodiment 488, wherein one R′ is phenyl.

493. The method of embodiment 488, wherein one R′ is substituted phenyl.

494. The method of any one of embodiments 489-493, wherein the other R′ is optionally substituted C₁₋₆ aliphatic.

495. The method of any one of embodiments 489-493, wherein the other R′ is optionally substituted C₁₋₆ alkyl.

496. The method of any one of embodiments 401-405, wherein the other R′ is optionally substituted phenyl.

497. The method of any one of embodiments 401-405, wherein the other R′ is phenyl.

498. The method of any one of embodiments 401-405, wherein the other R′ is substituted phenyl.

499. The method of any one of embodiments 479-498, wherein L′ is —C(R′)₂—.

500. The method of any one of embodiments 479-498, wherein L′ is optionally substituted —CH₂—.

501. The method of any one of embodiments 479-498, wherein L′ is —CH₂—.

502. The method of any one of embodiments 464-501, wherein the contact removes 2′-cyanoethyl.

503. The method of any one of embodiments 464-502, wherein the contact forms a natural phosphate linkage or a salt form thereof.

504. The method of any one of embodiments 364-502, comprising removing of another chiral auxiliary or group that having a different structure than that of any one of embodiments 470-502.

505. The method of any one of embodiments 364-502, comprising removing of

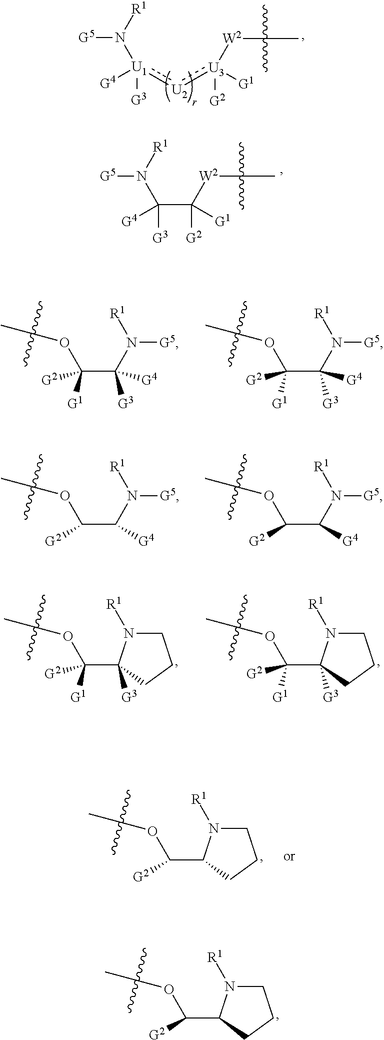

wherein G² is -L′-Si(R)₃, wherein each R is independently not —H.

506. The method of embodiment 505, wherein G² is —CH₂SiCH₃Ph₂.

507. The method of any one of embodiments 504-506, comprising contacting an oligonucleotide with a fluoride.

508. The method of any one of embodiments 504-506, comprising contacting an oligonucleotide with a solution comprising TEA-HF and a base.

509. The method of any one of embodiments 364-508, comprising cleaving oligonucleotide from a solid support.

510. The method of any one of embodiments 364-509, wherein the oligonucleotide or a composition thereof is an oligonucleotide or composition of any one of embodiments 1-347.

511. The compound of any one of embodiments 364-413, or a related diastereomer or enantiomer.

512. The method of any one of the preceding embodiments, wherein each heteroatom is independently boron, nitrogen, oxygen, silicon, sulfur, or phosphorus.

### EXEMPLIFICATION

The foregoing has been a description of certain non-limiting embodiments of the disclosure. Accordingly, it is to be understood that embodiments of the disclosure herein described are merely illustrative of applications of principles of the disclosure. Reference herein to details of illustrated embodiments is not intended to limit the scope of any claims.

Certain methods for preparing, and for assessing properties and/or activities of, oligonucleotides and oligonucleotide compositions are widely known in the art, including but not limited to those described in U.S. Pat. Nos. 9,394,333, 9,744,183, 9,605,019, 9,598,458, US 2015/0211006. US 2017/0037399, WO 2017/015555, WO 2017/192664, WO 2017/015575, WO2017/062862, WO 2017/160741, WO 2017/192679, and WO 2017/210647, the methods and reagents of each of which are incorporated herein by reference. Applicant describes herein example methods for preparing provided DMD oligonucleotides and DMD oligonucleotide compositions.

Functions and advantage of certain embodiments of the present disclosure may be more fully understood from the examples described below. The following examples are intended to illustrate certain benefits of such embodiments.

### Example 1. Example Synthesis of DMD Oligonucleotide Compositions

Certain technologies for preparing DMD oligonucleotide and compositions thereof are widely known in the art. In some embodiments, DMD oligonucleotides and DMD oligonucleotide compositions of the present disclosure were prepared using technologies, e.g., reagents (e.g., solid supports, coupling reagents, cleavage reagents, phosphoramidites, etc.), chiral auxiliaries, solvents (e.g., for reactions, washing, etc.), cycles, reaction conditions (e.g., time, temperature, etc.), etc., described in one or more of U.S. Pat. Nos. 9,394,333, 9,744,183, 9,605,019, 9,598,458, US 2015/0211006, US 2017/0037399, WO 2017/015555, WO 2017/192664, WO 2017/015575, WO2017/062862, WO 2017/160741, WO 2017/192679, WO 2017/210647, WO 2018/223056, WO 2018/237194, and WO 2019/055951.

### Example 2. Example Synthesis of Phosphoramidate Internucleotidic Linkages Comprising a Cyclic Guanidine Moiety

As illustrated herein, phosphoramidate internucleotidic linkages can be readily prepared from phosphite internucleotidic linkages, including stereopure phosphite internucleotidic linkages, in accordance with the present disclosure.

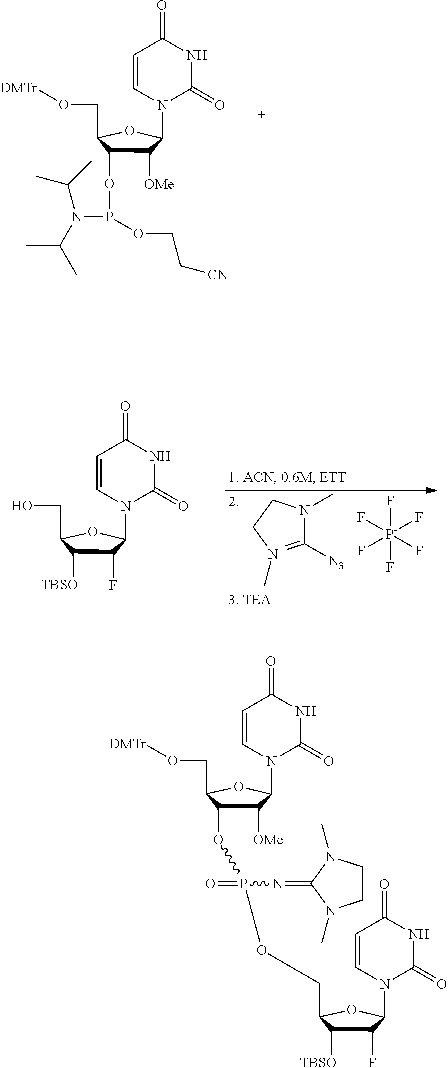

To a stirred solution of amidite (474 mg, 0.624 mmol, 1.5 equiv., pre-dried by co-evaporation with dry acetonitrile and under vacuum for a minimum of 12 h) and TBS protected alcohol (150 mg, 0.41 mmol, pre-dried by co-evaporation with dry acetonitrile and under vacuum for a minimum of 12 h) in dry acetonitrile (5.2 ml) was added 5-(ethylthio)-JH-tetrazole (ETT, 2.08 ml, 0.6M, 3 equiv.) under argon atmosphere at room temperature. The reaction mixture was stirred for 5 mins then monitored by LCMS and then a solution of 2-azido-1,3-dimethylimidazolinium hexafluorophosphate (356 mg, 1.24 mmol, 3 equiv.) in acetonitrile (1 ml) was added. Once the reaction was completed (after ~5 mins, monitored by LCMS) then triethylamine (0.17 ml, 1.24 mmol, 3 equiv) was added and the reaction was monitored by LCMS. The reaction mixture was concentrated under reduced pressure and then redissolved in dichloromethane (50 ml), washed with water (25 ml), saturated aq. sodium bicarbonate (25 ml), and brine (25 ml), and dried with magnesium sulfate. The solvent was removed under reduced pressure. The crude product was purified by silica gel column (80 g) using DCM (5% triethyl amine) and MeOH as eluent. Product-containing fractions were collected and the solvent was evaporated. The resulted product may contain TEA·HCl salt. To remove the salt, the product was re-dissolved in DCM (50 ml) and washed with saturated aq. sodium bicarbonate (20 ml) and brine (20 ml) then dried with magnesium sulfate and the solvent was evaporated. A pale yellow solid was obtained. Yield: 440 mg (89%). ³¹P NMR (162 MHz, CDCl₃) δ −1.34, −1.98. MS calculated for C₅₁H₆₅FN₇O₁₄PSi [M]⁺ 1078.17, Observed: 1078.57 [M+H]⁺.

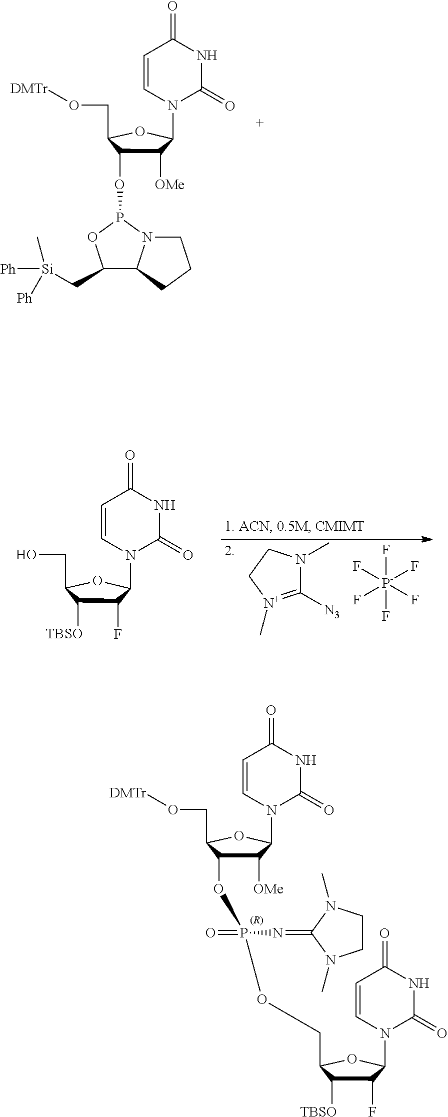

### Synthesis of Stereopure (Rp) Dimer.

To a stirred solution of L-DPSE chiral amidite (1.87 g, 2.08 mmol, 1.5 equiv., pre-dried by co-evaporation with dry acetonitrile and under vacuum for a minimum of 12 h) and TBS protected alcohol (500 mg, 1.38 mmol, pre-dried by co-evaporation with dry acetonitrile and under vacuum for a minimum of 12 h) in dry acetonitrile (18 mL) was added 2-(1H-imidazol-1-yl) acetonitrile trifluoromethanesulfonate (CMIMT, 5.54 mL, 0.5M, 2 equiv.) under argon atmosphere at room temperature. The resulting reaction mixture was stirred for 5 mins then monitored by LCMS and then a solution of 2-azido-1,3-dimethylimidazolinium hexafluorophosphate (1.18 g, 4.16 mmol, 3 equiv.) in acetonitrile (2 mL) was added. Once the reaction was completed (after ~5 mins, monitored by LCMS), the reaction mixture was concentrated under reduced pressure and then redissolved in dichloromethane (70 mL), washed with water (40 mL), saturated aq. sodium bicarbonate (40 mL) and brine (40 mL), and dried with magnesium sulfate. The solvent was removed under reduced pressure. The crude product was purified by silica gel column (120 g) using DCM (5% triethyl amine) and MeOH as eluent. Product containing fractions were collected and the solvent was evaporated. The resulted product contained TEA·HCl salt. To remove the salt, the product was re-dissolved in DCM (50 mL) and washed with saturated aq. sodium bicarbonate (20 mL) and brine (20 mL) and then dried with magnesium sulfate and the solvent was evaporated. A pale yellow foamy solid was obtained. Yield: 710 mg (47%). ³¹P NMR (162 MHz, CDCl₃) δ −1.38. MS calculated for C₅₁H₆₅FN₇O₁₄PSi [M]⁺ 1078.17, Observed: 1078.19.

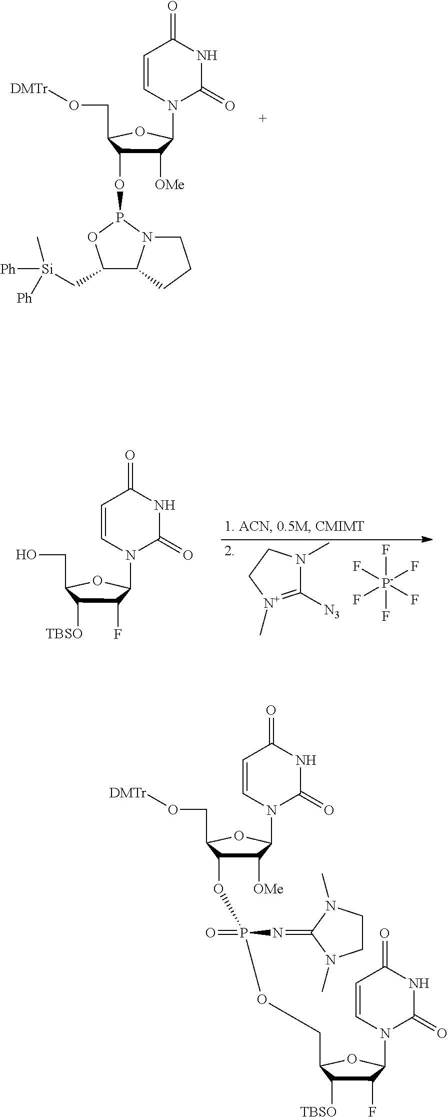

### Synthesis of Stereopure (Sp) Dimer

The same procedure was followed as for the Rp dimer. In place of L-DPSE chiral amidite, D-DPSE chiral amidite was used. A pale yellow foamy solid was obtained. Yield: 890 mg (59%). ³¹P NMR (162 MHz, CDCl₃) δ −1.93. MS calculated for C₅₁H₆₅FN₇O₁₄PSi [M]⁺ 1078.17, Observed: 1078.00.

In an example ³¹P NMR (internal standard of phosphoric acid at δ 0.0), the stereorandom preparation showed two peaks at −1.34 and −1.98, respectively; the stereopure Rp preparation showed a peak at −1.93, and the stereopure Sp preparation showed a peak at −1.38.

### Example 3. Preparation of DMD Oligonucleotides with Internucleotidic Linkages Comprising Neutral Guanidinium Group

In accordance with technologies described in the present disclosure, DMD oligonucleotides with various neutral and/or cationic internucleotidic linkages (e.g., at physiological pH) can be prepared. Illustrated below are preparation of DMD oligonucleotides comprising representative such internucleotidic linkages.

WV-11237 is a DMD oligonucleotide comprising four internucleotidic linkages having the structure of

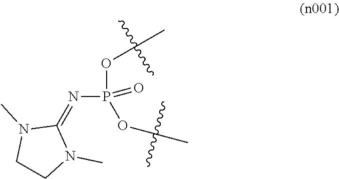 [SMILES: *OP(=O)(N=C1N(C)CCN1C)O*]

to introduce a neutral nature to the backbone and reduce the overall negative charges of the backbone. Expected molecular weight: 7113.4.

As an example, one preparation of WV-11237, including certain synthetic conditions and analytical results, is described below. Briefly, stereopure internucleotidic linkages were constructed using L-DPSE amidites and typical DPSE coupling cycles comprising Detritylation->Coupling->Pre-Cap->Thiolation->Post-Cap. Cycles for the n001 internucleotidic linkages were modified and comprised Detritylation->Coupling->Dimethyl imidazolium treatment->Post-cap. Compared to certain oxidation cycles, oxidation steps of oxidizing the P(III), e.g., with I₂-Pyridine-water, was replaced with the dimethyl imidazolium treatment.

Certain conditions and/or results of an example preparation.
Synthetic scale: 127 μmol

| Synthetic Steps | Conditions |
|---|---|
| Detritylation | 3% DCA in Toluene; 300 cm/hr, 436 UV watch |
| Coupling | 2.5 eq. of 0.2 M chiral amidite, 67% of 0.6 M CMIMT |
| | Recycle time: 10 min |
| Pre-Cap B | Reagent: 20:30:50::Acetic anhydride:Lutidine:Acetonitrile |
| | 1.5 CV, 3 min CT |
| Thiolation | Reagent: 0.2 M Xanthane Hydride |
| | 0.6 CV, 6 min CT |
| Capping (1:1 Cap A + Cap B) | 0.4 CV, 0.8 min CT |

Cap A = 20%:80% = NMI:ACN (v/v)
Cap B = 20%:30%:50% = Ac₂O:2,6-Lutidine:ACN (v/v/v)

| Synthetic Steps | Conditions |
|---|---|
| Detritylation | 3% DCA in Toluene; 300 cm/hr, |
| | 436 UV watch |
| Coupling | 2.5 eq. of 0.2 M standard amidite, 67% |
| | of 0.6 M ETTRecycle time: 8 min |
| Dimethyl imidazolium treatment: | 2.30 CV, 5 min CT, 3.5 eq. |
| Capping (1:1 Cap A + Cap B) | 0.4 CV, 0.8 min CT |

### Synthesis Process Parameters:

Synthesizer: AKTA Oligopilot 100
Solid Support: CPG 2′Fluoro-U, (85 umol/g)
Synthetic scale: 127 umol; 1.5 gm
Column diameter: 20 mm
Column volume: 6.3 mL

### Stereopure Coupling Reagents:

Monomer: 0.2M in MeCN (2′Fluoro-dA-L-DPSE, 2′Fluoro-dG-L-DPSE, 2′-OMe-A-L-DPSE); 0.2M in
20% isobutyronitrle/MeCN (2′Fluoro-dC-L-DPSE, 2′Fluoro-U-L-DPSE)
Deblocking: 3% DCA in Toluene
Activator: 0.6M CMIMT in MeCN
Sulfurization: 0.2M Xanthane Hydride in pyridine
Cap A: 20% NMI in MeCN
Cap B: Acetic anhydride, Lutidine, MeCN (20:30:50)
Pre-Cap: Neat Cap B

### Stereorandom Coupling Reagents:

Monomer: 0.2M in MeCN (2′OMeA and 2′OMeG)
Deblocking: 3% DCA in Toluene
Activator: 0.6M ETI in MeCN
2-Azido-1,3-dimethylimidazolinium-hexafluorophosphate: 0.1M in MeCN
Cap A: 20% NMI in MeCN
Cap B: Acetic anhydride, Lutidine, MeCN

### Deprotection Condition:

One pot deprotection by first treating the support with 5M TEA·HF in DMSO, H₂O, Triethylamine (pH 6.8). Incubation: 3 h, room temperature, 80 μL/μmol. Followed by addition of aqueous ammonia (200 μL/μmol). Incubation: 24 h, 35° C. The deprotected material was sterile filtered using 0.45 μm filters.
Yield: 72 O.D./μmol

### Recipe for 5× Solution of TEA·HF in DMSO/Water, 5/1, v/v:

| | | Volume | |
|---|---|---|---|
| Reagent | Solvents/Reagents | (mL) | Total Volume (mL) |
| (5×) TEA•HF | DMSO | 55.0 | 100 |
| in | Water | 11.0 | |
| DMSO/Water, | Triethylamine (TEA) | 9.0 | |
| 5/1, v/v | Triethylamine | 25.0 | |
| | trihydrofluoride | | |
| | (TEA•3HF) | | |

In an example crude UPLC chromatogram, there were four distinct peaks all having same desired molecular weight of 7113.2:

| | RT | Area | % Area | Height |
|---|---|---|---|---|
| 9 | 7.843 | 402732 | 16.75 | 212901 |
| 10 | 7.884 | 941388 | 39.14 | 327190 |
| 11 | 7.968 | 595232 | 24.75 | 275741 |
| 12 | 8.025 | 353090 | 14.68 | 150141 |

The example final QC UPLC chromatogram showed four distinct peaks all having the desired molecular weight of 7113.2 (% Purity 95.32). Crude LC-MS showed a single peak of desired molecular weight of 7113.2 (data not shown). The example final QC LC-MS showed a major peak with the desired molecular weight of 7113.1.

Other DMD oligonucleotides may be prepared using similar cycle conditions or variants thereof depending on specific chemistries of each DMD oligonucleotides.

### Example 4. Chirally Controlled Non-Negatively Charged Internucleotidic Linkages

### Dimer Synthesis.

This procedure is to make stereopure dimer phosphate backbone followed by incorporating it to selective sites of oligonucleotides (e.g., DMD oligonucleotides, single-stranded RNAi agents or ssRNA, etc.). A second approach is to synthesize molecules using an automated DMD oligonucleotide synthesizer to introduce a non-negatively charged internucleotidic linkage, e.g., a neutral internucleotidic linkage, at a specific site or full DMD oligonucleotide.

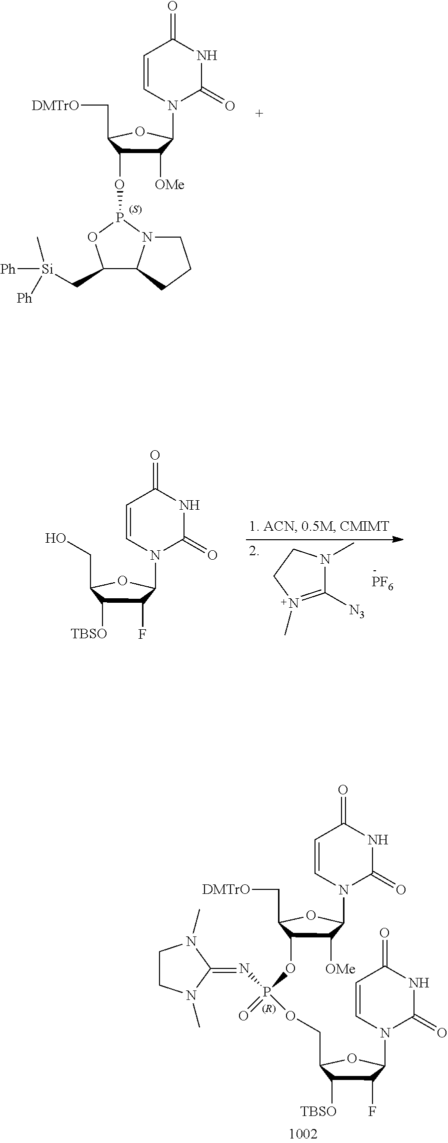

General experimental procedure (B) for stereopure (Rp) dimer: To a stirred solution of L (or) D-DPSE chiral amidite (1.87 g, 2.08 mmol, 1.5 equiv., pre-dried by co-evaporation with dry acetonitrile and kept it under vacuum for minimum 12 h) and TBS protected alcohol (500 mg, 1.38 mmol, pre-dried by co-evaporation with dry acetonitrile and kept it under vacuum for minimum 12 h) in dry acetonitrile (18 mL) was added 2-(1H-imidazol-1-yl) acetonitrile trifluoromethanesulfonate (CMIMT, 5.54 mL, 0.5M, 2 equiv.) under argon atmosphere at room temperature. Resulting reaction mixture was stirred for 5 mins then monitored by LCMS and then a solution of 2-azido-1,3-dimethylimidazolinium hexafluorophosphate (1.18 g, 4.16 mmol, 3 equiv.) in acetonitrile (2 mL) was added. Once the reaction was completed (after ~5 mins, monitored by LCMS) then the reaction mixture was concentrated under reduced pressure and then redissolved in dichloromethane (70 mL) washed with water (40 mL), saturated aq. sodium bicarbonate (40 mL) and brine (40 mL) dried with magnesium sulfate. Solvent was removed under reduced pressure. The crude product was purified by silica gel column (120 g) using DCM (2% triethyl amine) and MeOH as eluent. Product containing fractions are evaporated. Pale yellow foamy solid 1002 was obtained. Yield: 710 mg (47%). ³¹P NMR (162 MHz, CDCl₃) δ −1.38. MS (ES) m/z calculated for C₅₁H₆₅FN₇O₁₄PSi [M]⁺ 1077.40, Observed: 1078.19 [M+H]⁺.

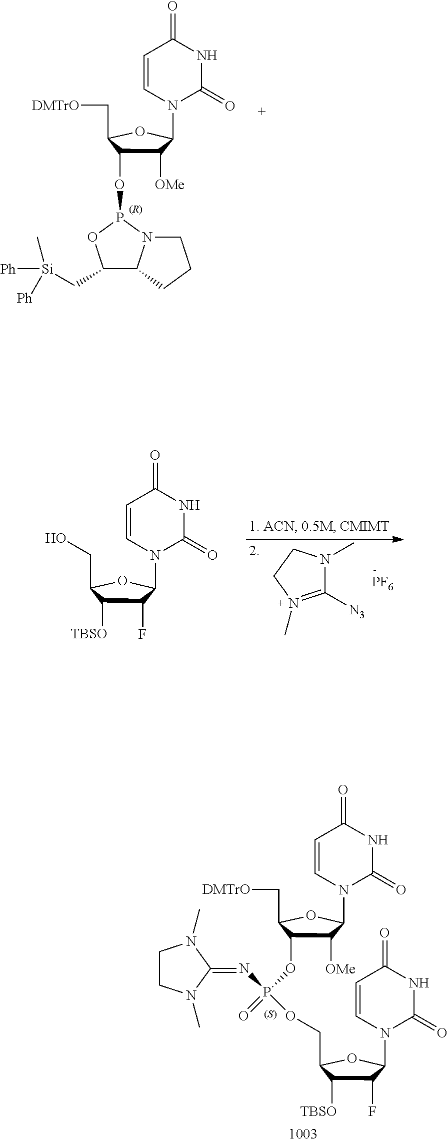

Stereopure (Sp) dimer 1003: The procedure B was followed as shown above. D-DPSE chiral amidite was used. Pale yellow foamy solid was obtained. Yield: 890 mg (59%). ³¹P NMR (162 MHz, CDCl₃) δ −1.93. MS (ES) m/z calculated for C₅₁H₆₅FN₇O₁₄PSi [M]⁺ 1077.40, Observed: 1078.00 [M+H]⁺.

General experimental procedure (C) for deprotection of TBS group: To a stirred solution of TBS protected compound (9.04 mmol) in THF (70 mL), was added TBAF (1.0 M, 13.6 mmol) at rt. The reaction mixture was stirred at room temperature for 2-4 h. LCMS showed there was no starting material left, then concentrated followed by purification using ISCO-combiflash system (330 g gold rediSep high performance silica column pre-equilibrated 3 CV with 2% TEA in DCM) and DCM/Methanol/2% TEA as a gradient eluent. Product containing column fractions were pooled together and evaporated followed by drying under high vacuum afforded the pure product.

General experimental procedure (D) for chiral amidites: The TBS deprotected compound (2.5 mmol) was dried by co-evaporation with 80 mL of anhydrous toluene (30 mL×2) at 35° C. and dried under at high vacuum for overnight. Then dried it was dissolved in dry THF (30 mL), followed by the addition of triethylamine (17.3 mmol) then the reaction mixture was cooled to −65° C. [for Guanine flavors: TMS-Cl, 2.5 mmol was added at −65° C., for non-Guanine flavors no TMS-Cl was added]. The THF solution of [(1R,3S,3aS)-1-chloro-3-((methyldiphenylsilyl)methyl)tetrahydro-1H,3H-pyrrolo[1,2-c][1,3,2]oxazaphosphole (or) (1S,3R,3aR)-1-chloro-3-((methyldiphenylsilyl)methyl)tetrahydro-1H,3H-pyrrolo[1,2-c][1,3,2]oxazaphosphole (1.8 equiv.) was added through syringe to the above reaction mixture over 2 min then gradually warmed to room temperature. After 20-30 min, at rt, TLC as well as LCMS indicated starting material was converted to product (reaction time: I h). Then the reaction mixture was filtered under argon using air free filter tube, washed with THF and dried under rotary evaporation at 26° C. afforded crude solid material, which was purified by ISCO-combiflash system (40 g gold rediSep high performance silica column (pre-equilibrated 3 CV with CH₃CN/5% TEA then 3 CV with DCM/5% TEA) using DCM/CH₃CN/5% TEA as a solvent (compound eluted at 10-40 DCM/CH₃CN/5% TEA). After evaporation of column fractions pooled together was dried under high vacuum afforded white solid to give isolated yield.

³¹P NMR (internal standard of Phosphoric acid at δ 0.0): 1001: −1.34 and −1.98. 1002: −1.93. 1003: −1.38. ¹H NMR of 1001, 1002, and 1003 demonstrated different chemical shifts for multiple hydrogens of the diastereomers. LCMS showed different retention times for the two diastereomers as well. Under one condition, the following retention times were observed: 1.90 and 2.15 for 1001, 1.92 for one diastereomer, and 2.17 for the other.

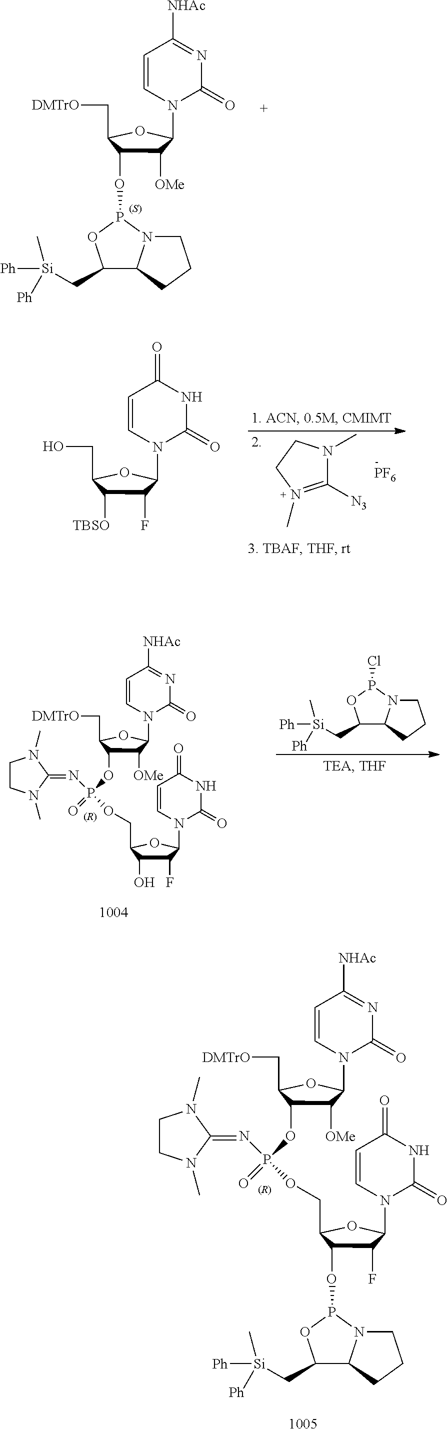

Compound 1004: Procedures B and C followed, Off-white foamy solid, Yield: (36%). ³¹P NMR (162 MHz, CDCl₃) δ −1.23. MS (ES) m/z calculated for C₄₇H₅₄FN₈O₁₄P [M]⁺ 1004.34, Observed: 1043.21 [M+K]⁺.

Compound 1005: Procedure D used, Off-white foamy solid, Yield: (81%). ³¹P NMR (162 MHz, CDCl₃) δ 154.43, −2.52. MS (ES) m/z calculated for C₆₆H₇₆FN₉O₁₅P₂Si [M]⁺ 1343.46, Observed: 1344.85 [M+H]⁺.

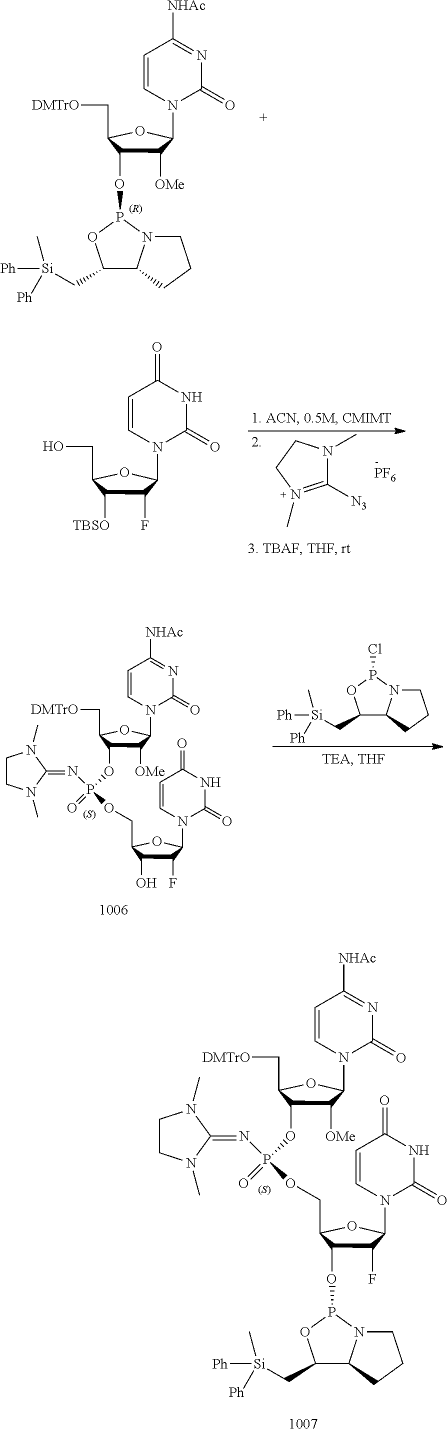

Compound 1006: Procedures B, and C followed, Off-white foamy solid, Yield: (47%). ³¹P NMR (162 MHz, CDCl₃) δ −2.54. MS (ES) m/z calculated for C₄₇H₅₄FN₈O₁₄P [M]⁺ 1004.34, Observed: 1043.12 [M+K]⁺.

Compound 1007: Procedure D used, Off-white foamy solid, Yield: (81%). ³¹P NMR (162 MHz, CDCl₃) δ 153.55, −2.20. MS (ES) m/z calculated for C₆₆H₇₆FN₉O₁₅P₂Si [M]⁺ 1343.46, Observed: 1344.75 [M+H]⁺.

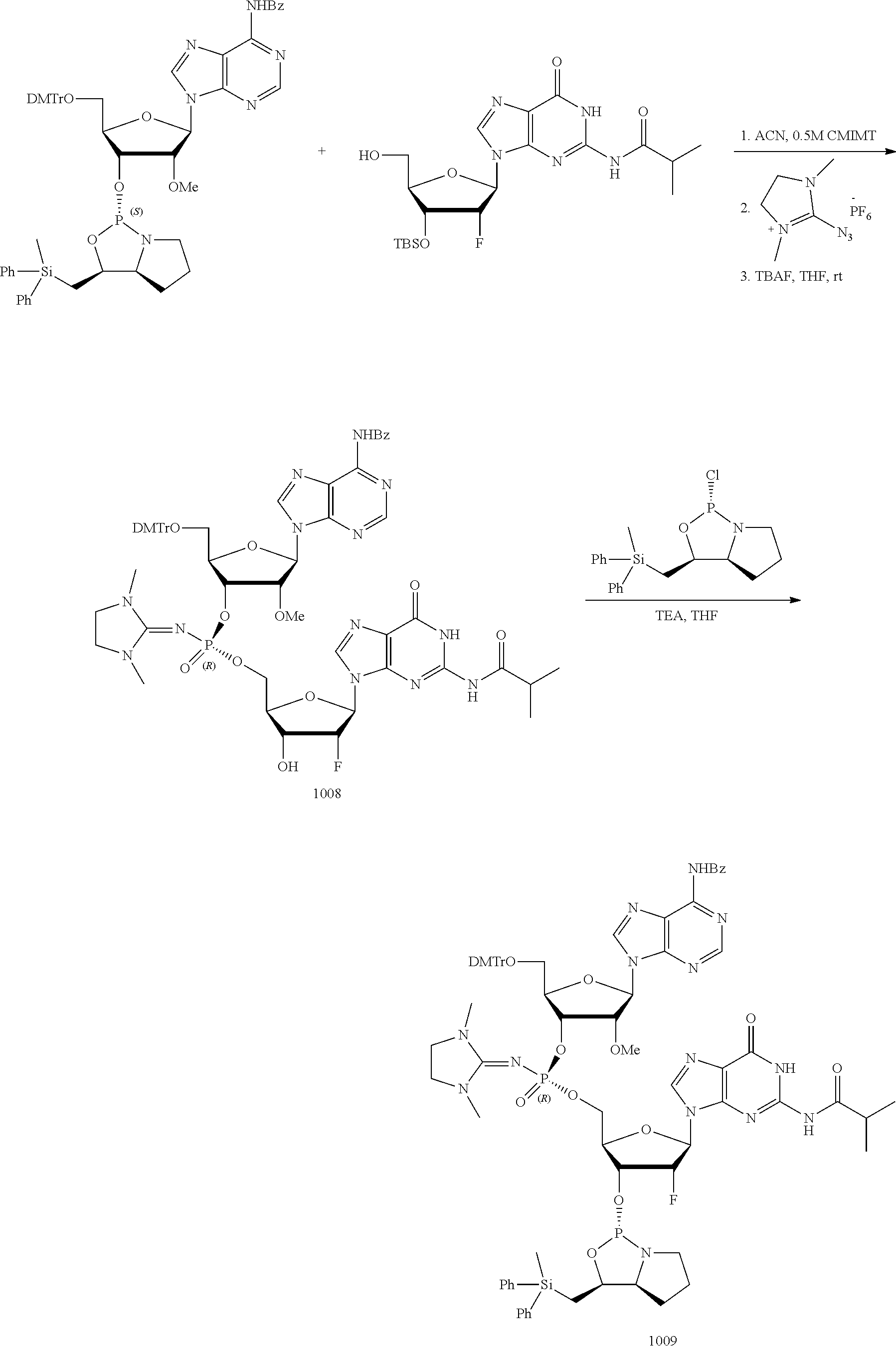

Compound 1008: Procedures B and C followed. Off-white foamy solid, Yield: (36%). ³¹P NMR (162 MHz, CDCl₃) δ −1.38. MS (ES) m/z calculated for C₃₈H₆₃FN₁₃O₁₃P [M]⁺ 1199.43, Observed: 1200.76 [M+H]⁺.

Compound 1009: Procedure D used. Off-white foamy solid, Yield: (60%). ³¹P NMR (162 MHz, CDCl₃) δ 157.26, −2.86. MS (ES) m/z calculated for C₇₇H₈₅FN₁₄O₁₄P₂Si [M]⁺ 1538.55, Observed: 1539.93 [M+H]⁺.

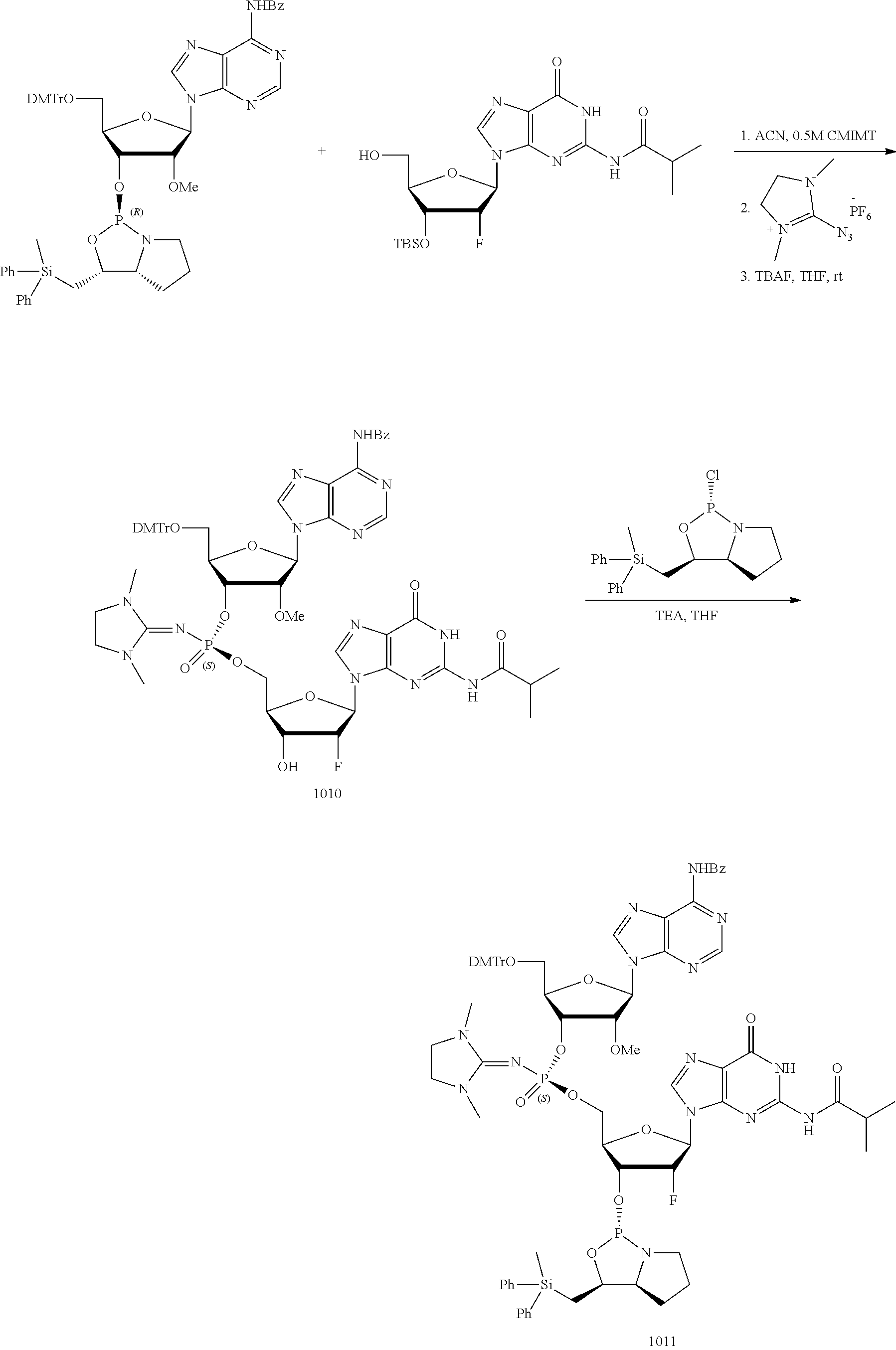

Compound 1010: Procedures B and C followed, Off-white foamy solid, Yield: (36%). ³¹P NMR (162 MHz, CDCl₃) δ −2.82. MS (ES) m/z calculated for C₅₈H₃FN₁₃O₁₃P [M]⁺ 1199.43, Observed: 1200.19 [M+H]⁺.

Compound 1011: Procedure D used, Off-white foamy solid, Yield: (63%). ³¹P NMR (162 MHz, CDCl₃) δ 159.56, −2.99. MS (ES) m/z calculated for C₇₇H₈₅FN₁₄O₁₄P₂Si [M]⁺ 1538.55, Observed: 1539.83 [M+H]⁺.

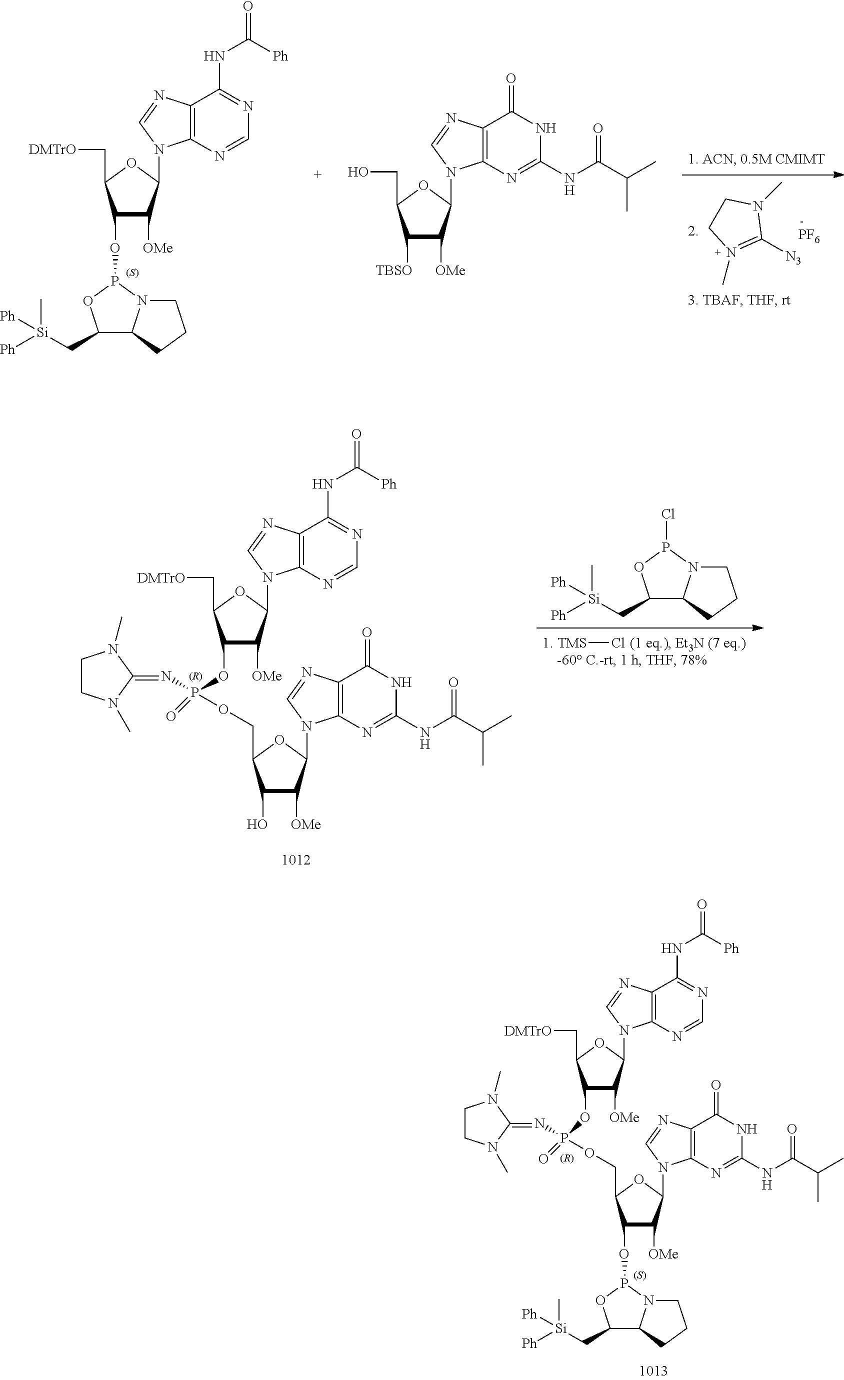

Compound 1012: Procedures B and C followed, Off-white foamy solid, Yield: (36%). [α]=−25.74 (c 1.06, CHCl₃). ³¹P NMR (162 MHz, Chloroform-d) δ −1.83. ¹H NMR (400 MHz, Chloroform-d) δ 12.14 (s, 1H), 11.28 (s, 1H), 9.15 (s, 1H), 8.56 (s, 1H), 8.25-7.94 (m, 2H), 7.90 (s, 1H), 7.72-7.48 (m, 2H), 7.44 (dd, J=8.2, 6.7 Hz, 2H), 7.35-7.26 (m, 2H), 7.24-7.02 (m, 8H), 6.81-6.56 (m, 4H), 6.04 (d, J=5.2 Hz, 1H), 5.67 (d, J=5.5 Hz, I H), 4.83 (dt, J=8.6, 4.4 Hz, l H), 4.71-4.54 (m, 2H), 4.49 (dt, J=14.2, 4.8 Hz, 2H), 4.35 (ddt, J=11.0, 5.1, 3.2 Hz, 1H), 4.28-4.09 (m, 2H), 3.68 (s, 6H), 3.37 (d, J=3.3 Hz, 7H), 3.33-3.17 (m, 5H), 2.82 (s, 5H), 2.74-2.60 (m, 1H), 1.92 (s, 2H), 1.72-1.50 (m, 1H), 1.08 (d, J=6.9 Hz, 3H), 0.94 (d, J=6.9 Hz, 3H). MS (ES) m/z calculated for C₅₉H₆₆N₁₃O₁₄P 1211.45 [M]⁺, Observed: 1212.42 [M+H]⁺.

Compound 1013: Procedure D used, Off-white foamy solid, Yield: (78%). [α]=−15.48 (c 0.96, CHCl₃). ³¹P NMR (162 MHz, Chloroform-d) δ 159.42, −2.47. MS (ES) m % z calculated for C₇₈H₈₈N₁₄O₁₅P₂Si 1550.57 [M]⁺, Observed: 1551.96 [M+H]⁺.

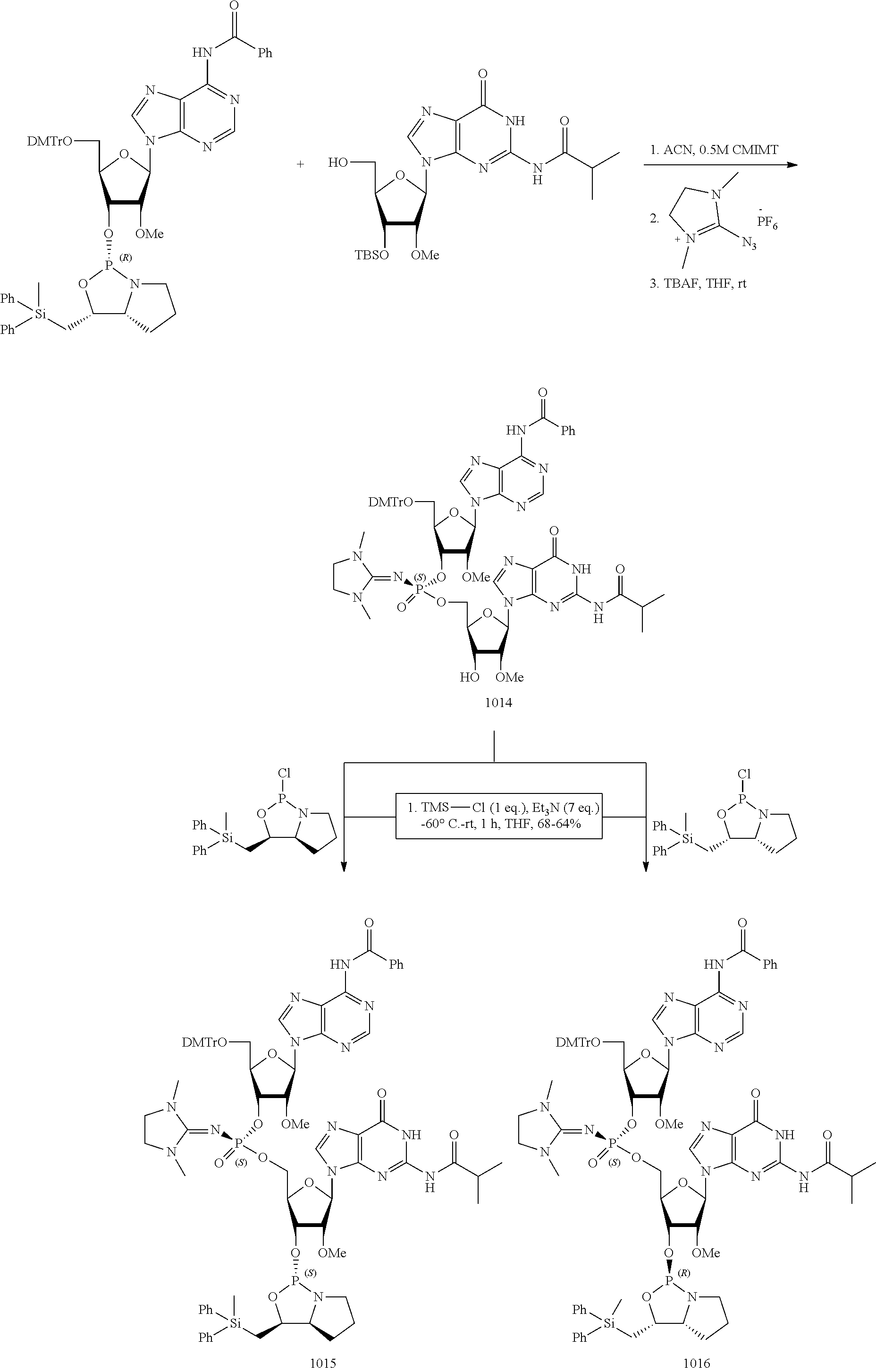

Compound 1014: Procedures B and C followed, Off-white foamy solid, Yield: (30%). [α]=−21.45 (c 0.55, CHCl₃). MS (ES) m/z calculated for C₅₉HN₁₃O₁₄P 1211.45 [M]⁺, Observed: 1212.80 [M+H]⁺.

Compound 1015: Procedure D used, Off-white foamy solid, Yield: (68%). [α]=−15.63 (c 1.44, CHCl₃). MS (ES) m/z Calculated for C₇₈H₈₈N₁₄O₁₅P₂Si 1550.57 [M]⁺, Observed: 1551.77 [M+H]⁺.

Compound 1016: Procedure D used, Off-white foamy solid, Yield: (64%). ³¹P NMR (162 MHz, CDCl₃) δ 156.64, −2.67. MS (ES) m/z Calculated for C₇₈H₈₈N₁₄O₁₅P₂Si 1550.57 [M]⁺, Observed: 1551.77 [M+H]⁺.

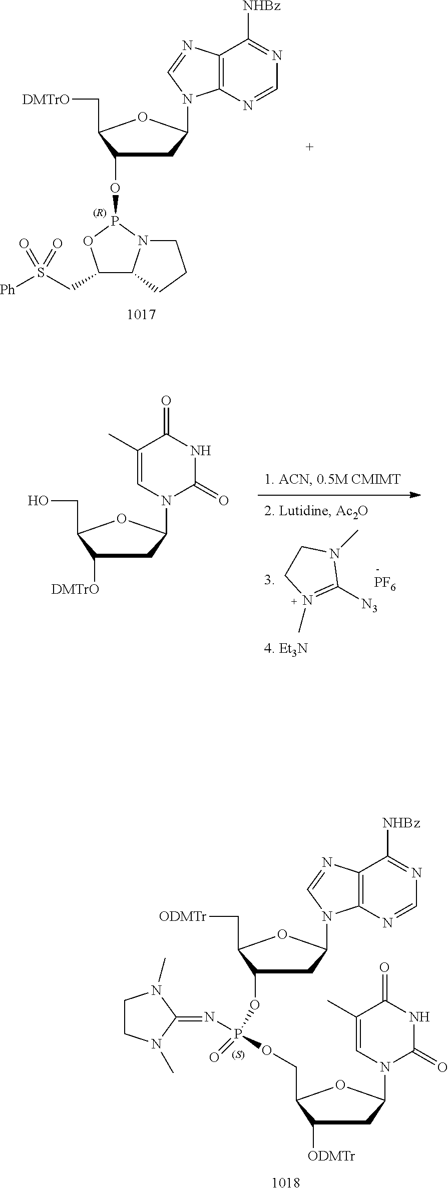

General experimental procedure (E) for stereopure dimer using sulfonyl amidite: To a stirred solution of stereopure sulfonyl amidite 1017 (259 mg, 0.275 mmol, 1.5 equiv) and TBS protected alcohol (100 mg, 0.18 mmol) in dry acetonitrile (2 mL) was added 2-(1H-imidazol-1-yl) acetonitrile trifluoromethanesulfonate (CMIMT, 0.73 mL, 0.36 mmol, 0.5M, 2 equiv.) under argon atmosphere at room temperature. Resulting reaction mixture was stirred for 5 mins and monitored by LCMS then a mixture of acetic anhydride (2M in ACN, 0.18 ml, 0.36 mmol, 2 equ) and lutidine (2M in ACN, 0.18 ml, 0.36 mmol, 2 equ) was added then stirred for ~5 mins then a solution of 2-azido-1,3-dimethylimidazolinium hexafluorophosphate (104.7 mg, 0.367 mmol, 2 equiv.) in acetonitrile (1 mL) was added. Once the reaction was completed (after ~5 mins, monitored by LCMS) then triethylamine (0.13 mL, 0.91 mmol, 5 equiv.) was added and monitored by LCMS. Once the reaction was completed, it was concentrated under reduced pressure and then re-dissolved in dichloromethane (50 mL) washed with water (25 mL), saturated aq. Sodium bicarbonate (25 mL) and brine (25 mL) dried with magnesium sulfate. Solvent was removed under reduced pressure. The crude product was purified by silica gel column (80 g) using DCM (2% triethylamine) and MeOH as eluent. Product containing fractions collected and evaporated. Off white solid 1018 obtained. Yield: 204 mg (82%). ³¹P NMR (162 MHz, CDCl₃) δ −1.87. MS (ES) m % z calculated for C₇₄H₇₅FN₁₀O₁₄P [M]⁺ 1359.44, Observed: 1360.39 [M+H]⁺.

Additional phosphoramidites that may be utilized for synthesis include:

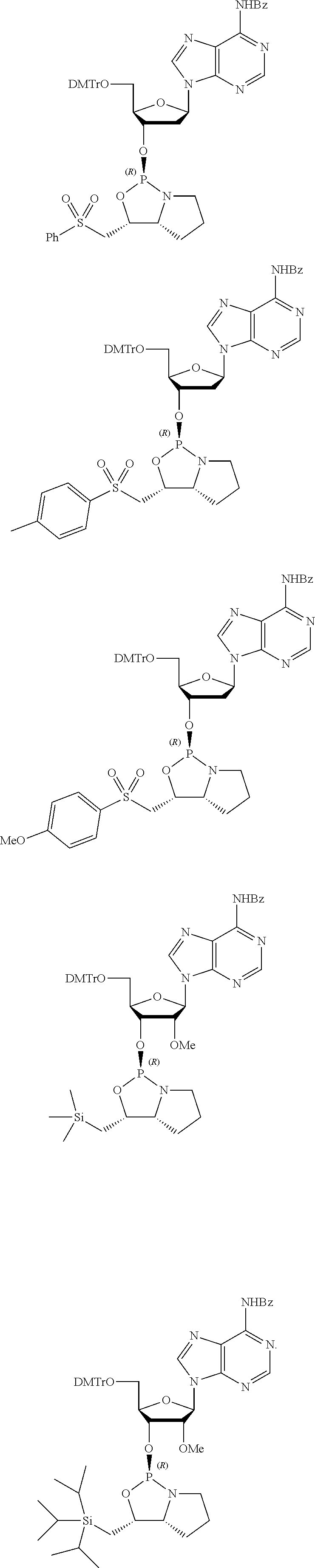

Additional useful chiral auxiliaries include:

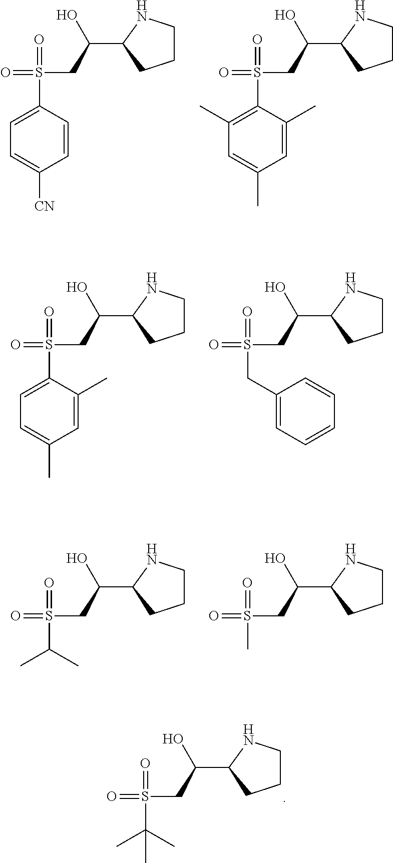

Other phosphoramidites and chiral auxiliaries, such as those described in U.S. Pat. Nos. 9,695,211, 9,605,019, 9,598,458, US 2013/0178612, US 20150211006, US 20170037399, WO 2017/015555, WO 2017/062862, WO 2017/160741, WO 2017/192664, WO 2017/192679, WO 2017/210647, WO 2018/098264, WO 2018/223056, and/or WO 2018/237194, the chiral auxiliaries and phosphoramidites of each of which is incorporated by reference.

### Example 5. Example Technologies for Chirally Controlled Oligonucleotide Preparation—Example Useful Chiral Auxiliaries

Among other things, the present disclosure provides technologies (e.g., chiral auxiliaries, phosphoramidites, cycles, conditions, reagents, etc.) that are useful for preparing chirally controlled internucleotidic linkages. In some embodiments, provided technologies are particularly useful for preparing certain internucleotidic linkages, e.g., non-negatively charged internucleotidic linkages, neutral internucleotidic linkages, etc., comprising P—N═, wherein P is the linkage. In some embodiments, the linkage phosphorus is trivalent. Certain example technologies (chiral auxiliaries and their preparations, phosphoramidites and their preparations, cycles, conditions, reagents, etc.) are described in the Examples herein. Among other things, such chiral auxiliaries provide milder reaction conditions, higher functional group compatibility, alternative deprotection and/or cleavage conditions, higher crude and/or purified yields, higher crude purity, higher product purity, and/or higher (or substantially the same or comparable) stereoselectivity when compared to a reference chiral auxiliary e.g. of formula O, P, Q, R or DPSE).

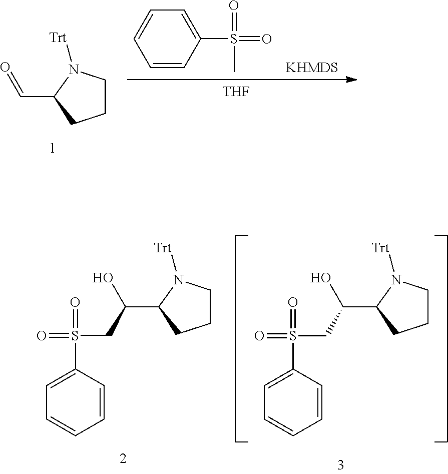

Two batches in parallel: To a solution of methylsulfonylbenzene (102.93 g, 658.96 mmol, 1.5 eq.) in THF (600 mL) was added KHMDS (1 M. 658.96 mL, 1.5 eq.) dropwise at −70° C., and warmed to −30° C. slowly over 30 min. The mixture was then cooled to −70° C. A solution of compound 1 (150 g, 439.31 mmol, 1 eq.) in THF (400 mL) was added dropwise at −70° C. The mixture was stirred at −70° C. for 3 hr. TLC (Petroleum ether: Ethyl acetate=3:1, Rf=0.1) indicated compound 1 was consumed completely and one major new spot with larger polarity was detected. Combined 2 batches. The reaction mixture was quenched by added to the sat. NH₄Cl (aq. 1000 mL), and then extracted with EtOAc (1000 mL×3). The combined organic layers were dried over Na₂SO₄, filtered, and concentrated under reduced pressure to give 1000 mL solution. Then added the MeOH (600 mL), concentrated under reduced pressure to give 1000 mL solution, then filtered the residue and washed with MeOH (150 mL); the residue was dissolved with THF (1000 mL) and MeOH (600 mL), then concentrated under reduced pressure to give 1000 mL solution. Then filtered to give a residue and washed with MeOH (150 mL). And repeat one more time. Compound 2 (248 g, crude) was obtained as a white solid. And the combined mother solution was concentrated under reduced pressure to give compound 3 (200 g, crude) as yellow oil.

Compound 2: ¹H NMR (400 MHz, CHLOROFORM-d) δ=7.80 (d, J=7.5 Hz, 2H), 7.74-7.66 (m, 1H), 7.61-7.53 (m, 2H), 7.47 (d, J=7.5 Hz, 6H), 7.24-7.12 (m, 9H), 4.50-4.33 (m, 1H), 3.33 (s, 1H), 3.26 (ddd, J=2.9, 5.2, 8.2 Hz, 1H), 3.23-3.10 (m, 2H), 3.05-2.91 (m, 2H), 1.59-1.48 (m, 1H), 1.38-1.23 (m, 1H), 1.19-1.01 (m, 1H), 0.31-0.12 (m, 1H).

### Preparation of Compound WV-CA-108.

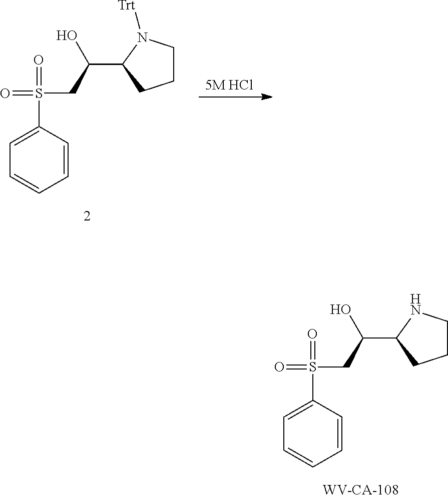

To a solution of compound 2 (248 g, 498.35 mmol, 1 eq.) in THF (1 L) was added HCl (5 M, 996.69 mL, 10 eq.). The mixture was stirred at 15° C. for 1 hr. TLC (Petroleum ether: Ethyl acetate=3:1, Rf=0.03) indicated compound 2 was consumed completely and one major new spot with larger polarity was detected. The resulting mixture was washed with MTBE (500 mL×3). The combined organic layers were back-extracted with water (100 mL). The combined aqueous layer was adjusted to pH 12 with 5M NaOH aq. and extracted with DCM (500 mL×3). The combined organic layers were dried over anhydrous Na₂SO₄, filtered and concentrated to afford a white solid. WV-CA-108 (122.6 g, crude) was obtained as a white solid.

¹H NMR (400 MHz, CHLOROFORM-d) δ=7.95 (d, J=7.5 Hz, 2H), 7.66 (t, J=7.5 Hz, 1H), 7.57 (t, J=7.7 Hz, 2H), 4.03 (ddd, J=2.6, 5.3, 8.3 Hz, 1H), 3.37-3.23 (m, 2H), 3.20-3.14 (m, 1H), 2.91-2.75 (m, 3H), 2.69 (br s, 1H), 1.79-1.54 (m, 5H); ¹³C NMR (101 MHz, CHLOROFORM-d) δ=139.58, 133.83, 129.28, 127.98, 67.90, 61.71, 59.99, 46.88, 25.98, 25.84; LCMS [M+H]⁺: 256.1. LCMS purity: 100%. SFC 100% purity.

### Preparation of Compound WV-CA-237.

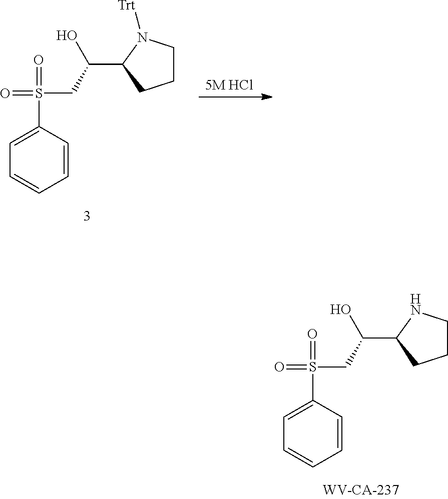

To a solution of compound 3 (400.00 g, 803.78 mmol) in THF (1.5 L) was added HCl (5 M, 1.61 L). The mixture was stirred at 15° C. for 2 hr. TLC indicated compound 3 was consumed completely and one major new spot with larger polarity was detected. The resulting mixture was washed with MTBE (500 mL×3). The combined aqueous layer was adjusted to pH 12 with 5M NaOH aq. and extracted with DCM (500 mL×1) and EtOAc (1000 mL×2). The combined organic layers were dried over anhydrous Na₂SO₄, filtered, and concentrated to afford as a brown solid. WV-CA-237 (100 g, crude) was obtained as a brown solid.

The residue was purified by column chromatography (SiO₂, Petroleum ether/Ethyl acetate=3/1 to Ethyl acetate:Methanol=1:2) to give 24 g crude. Then the 4 g residue was purified by prep-HPLC (column: Phenomenex luna C18 250×50 mm×10 um; mobile phase: [water (0.05% HCl)-ACN]; B %: 2%→20%, 15 min) to give desired compound (2.68 g, yield 65%,) as a white solid. WV-CA-237 (2.68 g) was obtained as a white solid. WV-CA-237: ¹H NMR (400 MHz, CHLOROFORM-d) δ=7.98-7.88 (m, 2H), 7.68-7.61 (m, 1H), 7.60-7.51 (m, 2H), 4.04 (dt, J=2.4, 5.6 Hz, 1H), 3.85 (ddd, J=3.1, 5.6, 8.4 Hz, 1H), 3.37-3.09 (m, 3H), 2.95-2.77 (m, 3H), 1.89-1.53 (m, 4H), 1.53-1.39 (m, 1H); ¹³C NMR (101 MHz, CHLOROFORM-d) δ=139.89, 133.81, 133.70, 129.26, 129.16, 128.05, 127.96, 68.20, 61.77, 61.61, 61.01, 60.05, 46.67, 28.02, 26.24, 25.93; LCMS [M+H]⁺: 256.1. LCMS purity: 80.0%. SFC dr=77.3:22.7.

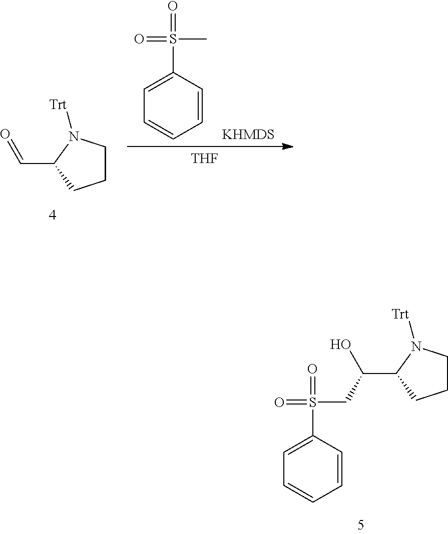

To a solution of compound 4 (140 g, 410.02 mmol) in THF (1400 mL) was added methylsulfonylbenzene (96.07 g, 615.03 mmol), then added KHMDS (1 M, 615.03 mL) in 0.5 hr. The mixture was stirred at −70~−40° C. for 3 hr. TLC indicated compound 4 was consumed and one new spot formed. The reaction mixture was quenched by addition sat. NH₄Cl aq. 3000 mL at 0° C., and then diluted with EtOAc (3000 mL) and extracted with EtOAc (2000 mL×3). Dried over Na₂SO₄, filtered, and concentrated under reduced pressure to give a residue. To the crude was added THF (1000 mL) and MeOH (1500 mL), concentrated under reduced pressure at 45° C. until about 1000 mL residue remained, filtered the solid. Repeat 3 times. Compound 5 (590 g, 72.29% yield) was obtained as a yellow solid. ¹H NMR (400 MHz, CHLOROFORM-d) δ=7.81 (d, J=7.5 Hz, 2H), 7.75-7.65 (m, 1H), 7.62-7.53 (m, 2H), 7.48 (br d, J=7.2 Hz, 6H), 7.25-7.11 (m, 9H), 4.50-4.37 (m, 1H), 3.31-3.11 (m, 3H), 3.04-2.87 (m, 2H), 1.60-1.48 (m, 1H), 1.39-1.24 (m, 1H), 1.11 (dtd, J=4.5, 8.8, 12.8 Hz, 1H), 0.32-0.12 (m, 1H).

### Preparation of Compound WV-CA-236.

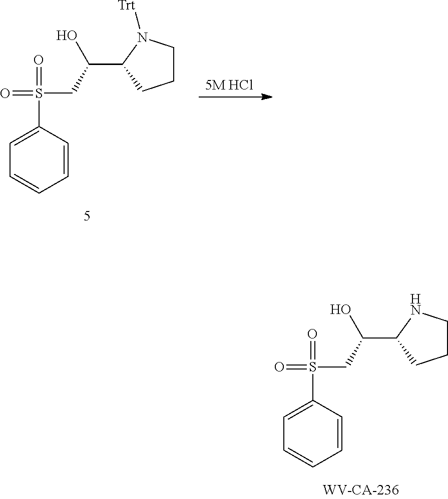

To a solution of compound 5 (283 g, 568.68 mmol) in THF (1100 mL) was added HCl (5 M, 1.14 L). The mixture was stirred at 25° C. for 2 hr. TLC indicated compound 5 was consumed and two new spots formed. The reaction mixture was washed with MTBE (1000 mL×3), then the aqueous phase was basified by addition NaOH (5M) until pH=12 at 0° C., and then extracted with DCM (1000 mL×3) to give a residue, dried over Na₂SO₄, filtered, and concentrated under reduced pressure to give a residue. Compound WV-CA-236 (280 g, 1.10 mol, 96.42% yield) was obtained as a yellow solid.

The crude product was added HCl/EtOAc (1400 mL, 4M) at 0° C., 2 hr later, filtered the white solid and washed the solid with MeOH (1000 mL×3). LCMS showed the solid contained another peak (MS=297). Then the white solid was added H₂O (600 mL) and washed with DCM (300 mL×3). The aqueous phase was added NaOH (5 M) until pH=12. Then diluted with DCM (800 mL) and extracted with DCM (800 mL×4). The combined organic layer was dried over Na₂SO₄, filtered, and concentrated under reduced pressure to give the product. Compound WV-CA-236 (280 g) was obtained as a yellow solid. ¹H NMR (400 MHz, CHLOROFORM-d) 5=8.01-7.89 (m, 2H), 7.69-7.62 (m, 1H), 7.61-7.51 (m, 2H), 4.05 (ddd, J=2.8, 5.2, 8.4 Hz, 1H), 3.38-3.22 (m, 2H), 3.21-3.08 (m, 1H), 2.95-2.72 (m, 4H), 1.85-1.51 (m, 4H); ¹³C NMR (101 MHz, CHLOROFORM-d) δ=139.75, 133.76, 129.25, 127.94, 67.57, 61.90, 60.16, 46.86, 25.86. LCMS [M+H]⁺: 256. LCMS purity: 95.94. SFC purity: 99.86%.

### Example 6. Example Technologies for Chirally Controlled Oligonucleotide Preparation—Example Useful Phosphoramidites

Among other things, the present disclosure provides phosphoramidites useful for oligonucleotide synthesis. In some embodiments, provided phosphoramidites are particularly useful for preparation of chirally controlled internucleotidic linkages. In some embodiments, provided phosphoramidites are particularly useful for preparing chirally controlled internucleotidic linkages, e.g., non-negatively charged internucleotidic linkages or neutral internucleotidic linkages, etc., that comprise P—N═. In some embodiments, the linkage phosphorus is trivalent. In some embodiments, the linkage phosphorus is pentavalent.

General Procedure I for Chlorotriazine: In some embodiments, in an example procedure, a chiral auxiliary (174.54 mmol) was dried by azeotropic evaporation with anhydrous toluene (80 mL×3) at 35° C. in a rota-evaporator and dried under high vacuum for overnight. A solution of this dried chiral auxiliary (174.54 mmol) and 4-methylmorpholine (366.54 mmol) dissolved in anhydrous THF (200 mL) was added to an ice-cooled (isopropyl alcohol-dry ice bath) solution of trichlorophosphine (37.07 g, 16.0 mL, 183.27 mmol) in anhydrous THF (150 mL) placed in three neck round bottomed flask through cannula under Argon (start Temp: −10.0° C., Max: temp 0° C., 28 min addition) and the reaction mixture was warmed at 15° C. for 1 hr. After that the precipitated white solid was filtered by vacuum under argon using airfree filter tube (Chemglass: Filter Tube, 24/40 Inner Joints, 80 mm OD Medium Frit, Airfree, Schlenk). The solvent was removed with rota-evaporator under argon at low temperature (25° C.) and the crude semi-solid obtained was dried under vacuum overnight (-15 h) and was used for the next step directly.

General Procedure I for Chlorotriazine: In some embodiments, in an example procedure, a chiral auxiliary (174.54 mmol) was dried by azeotropic evaporation with anhydrous toluene (80 mL×3) at 35° C. in a rota-evaporator and dried under high vacuum for overnight. A solution of this dried chiral auxiliary (174.54 mmol) and 4-methylmorpholine (366.54 mmol) dissolved in anhydrous THF (200 mL) was added to an ice-cooled (isopropyl alcohol-dry ice bath) solution of trichlorophosphine (37.07 g, 16.0 mL, 183.27 mmol) in anhydrous THF (150 mL) placed in three neck round bottomed flask through cannula under Argon (start Temp: −10.0° C., Max: temp 0° C., 28 min addition) and the reaction mixture was warmed at 15° C. for 1 hr. After that the precipitated white solid was filtered by vacuum under argon using airfree filter tube (Chemglass: Filter Tube, 24/40 Inner Joints, 80 mm OD Medium Frit, Airfree, Schlenk). The solvent was removed with rota-evaporator under argon at low temperature (25° C.) and the crude semi-solid obtained was dried under vacuum overnight (-15 h) and was used for the next step directly.

General Procedure III for Coupling: In some embodiments, in an example procedure, a nucleoside (9.11 mmol) was dried by co-evaporation with 60 mL of anhydrous toluene (60 mL×2) at 35° C. and dried under high vacuum for overnight. The dried nucleoside was dissolved in dry THF (78 mL), followed by the addition of triethylamine (63.80 mmol) and then cooled to −5° C. under Argon (for 2′F-dG/2′OMe-dG case 0.95 eq of TMS-Cl used). The THF solution of the crude (made from general procedure I (or) II, 14.57 mmol), was added through cannula over 3 min then gradually warmed to room temperature. After 1 hr at room temperature, TLC indicated conversion of SM to product (total reaction time I h), the reaction mixture was then quenched with H₂O (4.55 mmol) at 0° C., and anhydrous MgSO₄ (9.11 mmol) was added and stirred for 10 min. Then the reaction mixture was filtered under argon using airfree filter tube, washed with THF, and dried under rotary evaporation at 26° C. to afford white crude solid product, which was dried under high vacuum overnight. The crude product was purified by ISCO-Combiflash system (rediSep high performance silica column pre-equilibrated with Acetonitrile) using Ethyl acetate/Hexane with 1% TEA as a solvent (compound eluted at 100% EtOAc/Hexanes/1% Et₃N) (for 2′F-dG case Acetonitrile/Ethyl acetate with 1% TEA used). After evaporation of column fractions pooled together, the residue was dried under high vacuum to afford the product as a white solid.

### Preparation of Amidites (1030-1039).

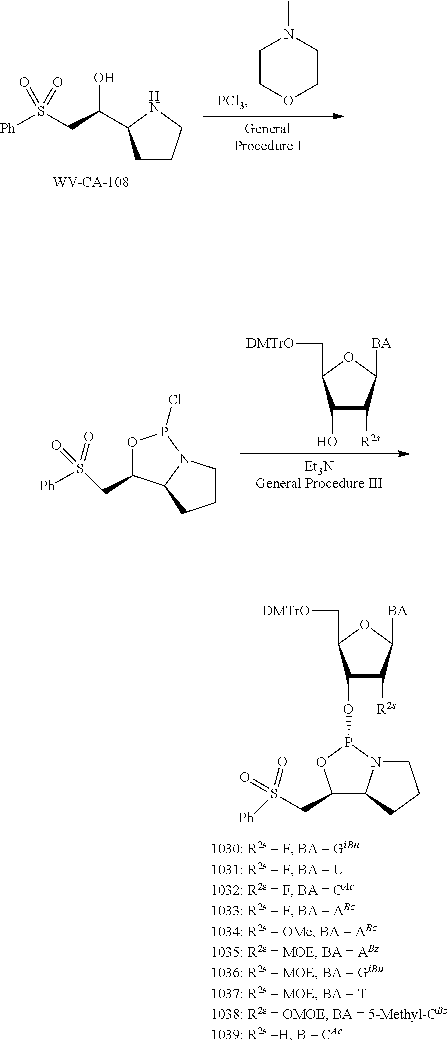

Preparation of 1030: General Procedure I followed by General Procedure III used. Off-white foamy solid. Yield: (73%). ³¹P NMR (162 MHz, CDCl₃) δ 153.32. (ES) m/z Calculated for C₄₇H₅₀FN₆O₁₀PS: 940.98 [M]⁺, Observed: 941.78 [M+H]⁺.

Preparation of 1031: General Procedure I followed by General Procedure III used. Off-white foamy solid. Yield: (78%). ³¹P NMR (162 MHz, CDCl₃) δ 153.62. (ES) m/z Calculated for C₄₂H₄₃FN₃O₁₀PS: 831.85 [M]⁺, Observed: 870.58 [M+K]⁺.

Preparation of 1032: General Procedure I followed by General Procedure III used. Off-white foamy solid. Yield: (68%). ³¹P NMR (162 MHz, CDCl₃) δ 153.95. (ES) m % z Calculated for C₄₄H₄₆FN₄O₁₀PS: 872.26 [M]⁺, Observed: 873.62 [M+H]⁺.

Preparation of 1033: General Procedure I followed by General Procedure III used, white foamy solid. Yield: (87%). ³¹P NMR (162 MHz, CDCl₃) δ 151.70. (ES) m/z Calculated for C₅₀H₄₈FN₆O₉PS: 958.29 [M]⁺, Observed: 959.79, 960.83 [M+H]⁺.

Preparation of 1034: General Procedure I followed by General Procedure III used. Off-white foamy solid. Yield: (65%). ³¹P NMR (162 MHz, CDCl₃) δ 154.80. (ES) m/z Calculated for C₅₁H₅₁N₆O₁₀PS: 971.31 [M]⁺, Observed: 971.81 [M+H]⁺.

Preparation of 1035: General Procedure I followed by General Procedure III used. Off-white foamy solid. Yield: (76%). ³¹P NMR (162 MHz, CDCl₃) δ 156.50. (ES) m/z Calculated for C₅₃H₅₅N₆O₁₁PS: 1014.33 [M]⁺, Observed: 1015.81 [M+H]⁺.

Preparation of 1036: General Procedure I followed by General Procedure III used. Off-white foamy solid. Yield: (78%). ³¹P NMR (162 MHz, CDCl₃) δ 156.40. (ES) m/z Calculated for C₅₀H₅₇N₆O₁₂PS: 996.34 [M]⁺, Observed: 997.90 [M+H]⁺.

Preparation of 1037: General Procedure I followed by General Procedure III used. Off-white foamy solid. Yield: (73%). ³¹P NMR (162 MHz, CDCl₃) δ 154.87. (ES) m/z Calculated for C₄₆H₅₂N₃O₁₂PS: 901.30 [M]⁺, Observed: 940.83 [M+K]⁺.

Preparation of 1038: General Procedure I followed by General Procedure III used. Off-white foamy solid. Yield: (75%). ³¹P NMR (162 MHz, CDCl₃) δ 154.94. (ES) m/z Calculated for C₅₃H₅₇N₄O₁₂PS: 1004.34 [M]⁺, Observed: 1005.86 [M+H]⁺.

Preparation of 1039: General Procedure I followed by General Procedure III used. Off-white foamy solid. Yield: (80%). ³¹P NMR (162 MHz, CDCl₃) δ 153.52. (ES) m/z Calculated for C₄₄H₄₇N₄O₁₀PS: 854.28 [M]⁺, Observed: 855.41 [M+H]⁺.

### Preparation of Amidites (1040-1049).

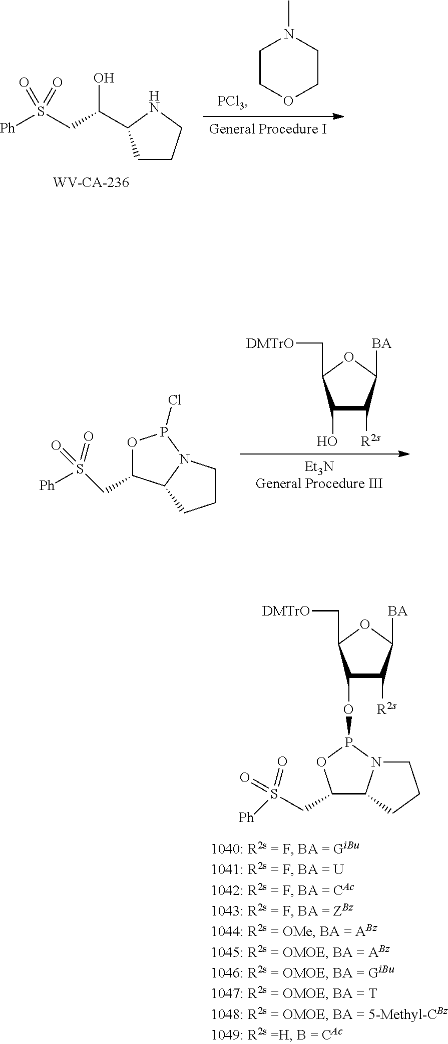

Preparation of 1040: General Procedure I followed by General Procedure III used. Off-white foamy solid. Yield: (78%). ³¹P NMR (162 MHz, CDCl₃) δ 157.80. (ES) m/z Calculated for C₄₇H₅₀FN₆O₁₀PS: 940.98 [M]⁺, Observed: 941.68 [M+H]⁺.

Preparation of 1041: General Procedure I followed by General Procedure III used. Off-white foamy solid. Yield: (78%). ³¹P NMR (162 MHz, CDCl₃) δ 157.79. (ES) m/z Calculated for C₄₂H₄₃FN₃O₁₀PS: 831.85 [M]⁺, Observed: 870.68 [M+K]⁺.

Preparation of 1042: General Procedure I followed by General Procedure III used. Off-white foamy solid. Yield: (78%). P NMR (162 MHz, CDCl₃) δ 158.07. (ES) m/z Calculated for C₄₄H₄₆FN₄O₁₀PS: 872.26 [M]⁺, Observed: 873.62 [M+H]⁺.

Preparation of 1043: General Procedure I followed by General Procedure III used, white foamy solid. Yield: (86%). ³¹P NMR (162 MHz, CDCl₃) δ 156.48. (ES) m/z Calculated for C₅₀H₄₈FN₆O₉PS: 958.29 [M]⁺, Observed: 959.79, 960.83 [M+H]*.

Preparation of 1044: General Procedure I followed by General Procedure III used. Off-white foamy solid. Yield: (65%). ³¹P NMR (162 MHz, CDCl₃) δ 154.80. (ES) m-z Calculated for C₅₁H₅₁MN₆O₁₀PS: 971.31 [M]⁺, Observed: 971.81 [M+H]⁺.

Preparation of 1045: General Procedure I followed by General Procedure III used. Off-white foamy solid. Yield: (77%). ³¹P NMR (162 MHz, CDCl₃) δ 154.74. (ES) m/z Calculated for C₅₃H₅₅N₆O₁₁PS: 1014.33 [M]⁺, Observed: 1015.81 [M+H]⁺.

Preparation of 1046: General Procedure I followed by General Procedure III used. Off-white foamy solid. Yield: (76%). ³¹P NMR (162 MHz, CDCl₃) δ 155.05. (ES) m/z Calculated for C₅₀H₅₇N₆O₁₂PS: 996.34 [M]⁺, Observed: 997.90 [M+H]⁺.

Preparation of 1047: General Procedure I followed by General Procedure III used. Off-white foamy solid. Yield: (75%). ³¹P NMR (162 MHz, CDCl₃) δ 155.44. (ES) m/z Calculated for C₄₆H₅₂N₃O₁₂PS: 901.30 [M]⁺, Observed: 940.83 [M+K]⁺.

Preparation of 1048: General Procedure I followed by General Procedure III used. Off-white foamy solid. Yield: (73%). ³¹P NMR (162 MHz, CDCl₃) δ 155.96. (ES) m/z Calculated for C₅₃H₅₇N₄O₁₂PS: 1004.34 [M]⁺, Observed: 1005.86 [M+H]⁺.

Preparation of 1049: General Procedure I followed by General Procedure III used. Off-white foamy solid. Yield: (80%). ³¹P NMR (162 MHz, CDCl₃) δ 156.37. (ES) m/z Calculated for C₄₄H₄₇N₄O₁₀PS: 854.28 [M]⁺, Observed: 855.31 [M+H]⁺.

### Example 7. Example Technologies for Chirally Controlled Oligonucleotide Preparation—Example Cycles, Conditions and Reagents for Oligonucleotide Synthesis

In some embodiments, the present disclosure provides technologies (e.g., reagents, solvents, conditions, cycle parameters, cleavage methods, deprotection methods, purification methods, etc.) that are particularly useful for preparing chirally controlled internucleotidic linkages. In some embodiments, such internucleotidic linkages, e.g., non-negatively charged internucleotidic linkages or neutral internucleotidic linkages, etc., comprise P—N═, wherein P is the linkage phosphorus. In some embodiments, the linkage phosphorus is trivalent. In some embodiments, the linkage phosphorus is pentavalent. As demonstrated herein, technologies of the present disclosure can provide mild reaction conditions, high functional group compatibility, alternative deprotection and/or cleavage conditions, high crude and/or purified yields, high crude purity, high product purity, and/or high stereoselectivity.

In some embodiments, a cycle for preparing natural phosphate linkages comprises or consists of deprotection (e.g., detritylation), coupling, oxidation (e.g., using I₂/Pyr/Water or other suitable methods available in the art) and capping (e.g., cap 2 described herein or other suitable methods available in the art). An example cycle is depicted below, wherein B1 and B2 are independently nucleobases. As appreciated by those skilled in the art, various modifications, e.g., sugar modifications, base modifications, etc. are compatible and may be included.

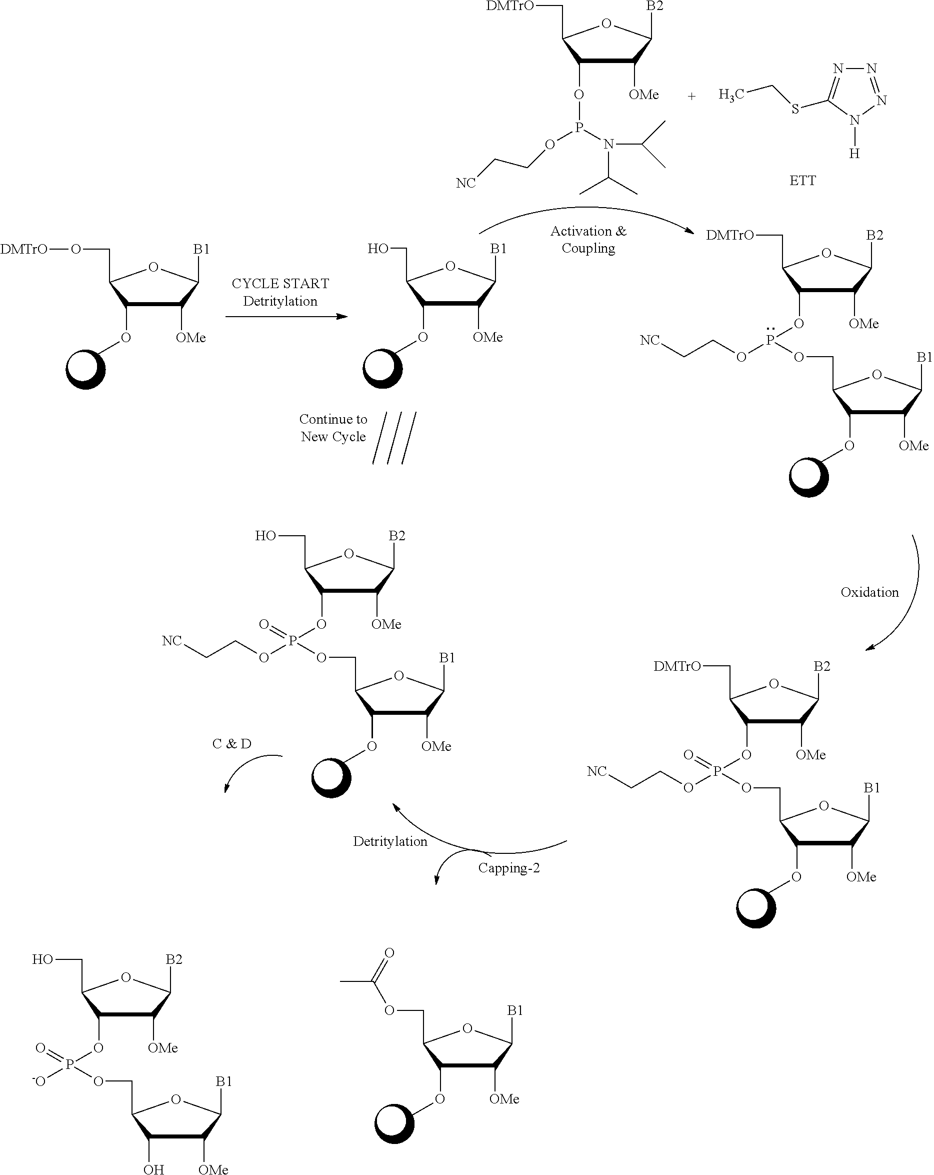

In some embodiments, a cycle for preparing non-natural phosphate linkages (e.g., phosphorothioate internucleotidic linkages) comprises or consists of deprotection (e.g., detritylation), coupling, a first capping (e.g., capping-1 as described herein), modification (e.g., thiolation using XH or other suitable methods available in the art), and a second capping (e.g., capping-2 as described herein or other suitable methods available in the art). An example cycle is depicted below, wherein B1 and B2 are independently nucleobases. As appreciated by those skilled in the art, various modifications, e.g., sugar modifications, base modifications, etc. are compatible and may be included. In some embodiments, a cycle using a DPSE chiral auxiliary is referred to as a DPSE cycle or DPSE amidite cycle.

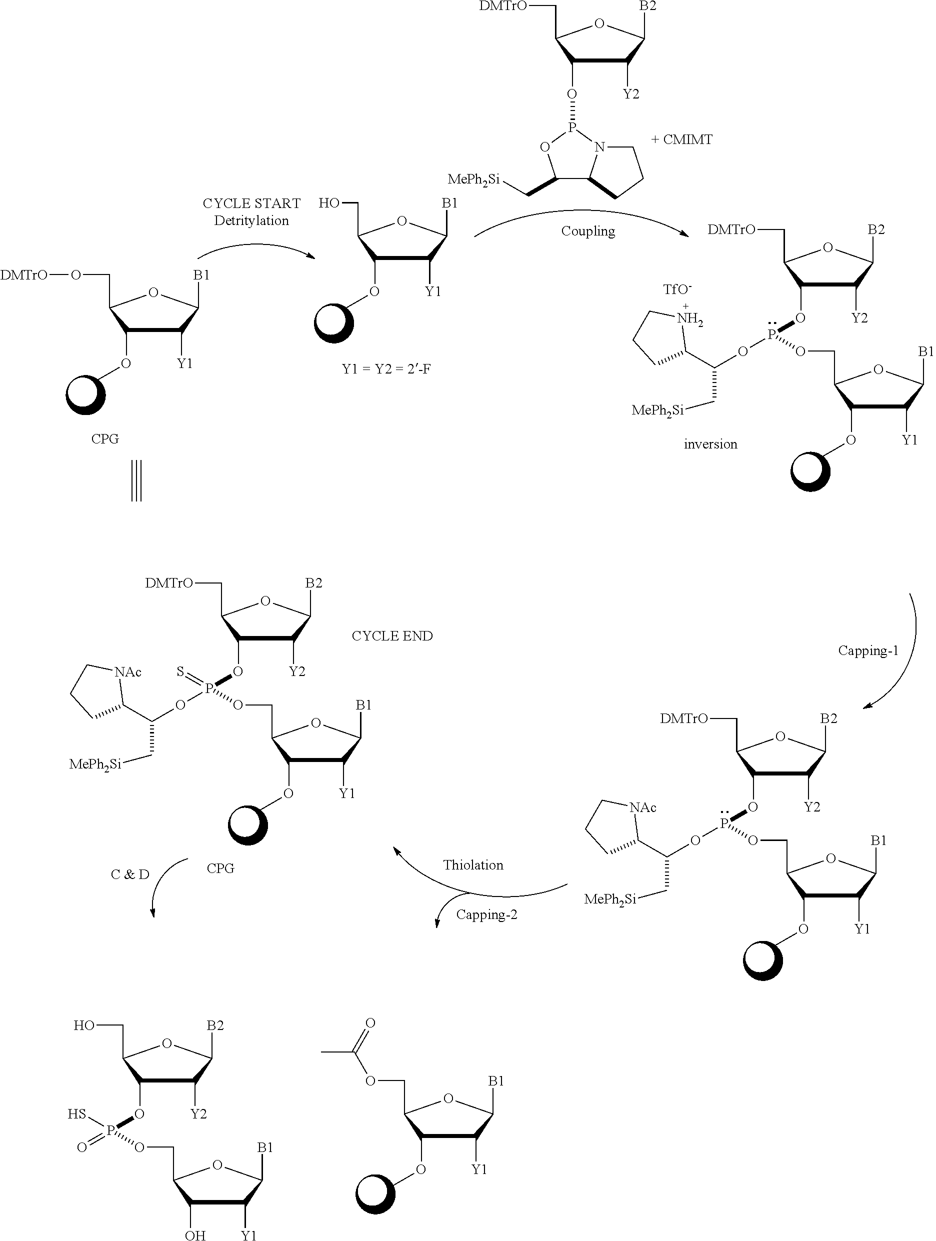

In some embodiments, a cycle for preparing non-natural phosphate linkages (e.g., certain non-negatively charged internucleotidic linkages, neutral internucleotidic linkages, etc.) or a salt form thereof, comprises or consists of deprotection (e.g., detritylation), coupling, a first capping (e.g., capping-1 as described herein), modification (e.g., using ADIH or other suitable methods available in the art), and a second capping (e.g., capping-2 as described herein or other suitable methods available in the art). An example cycle is depicted below, wherein B1 and B2 are independently nucleobases. As appreciated by those skilled in the art, various modifications, e.g., sugar modifications, base modifications, etc. are compatible and may be included. In some embodiments, a cycle using a PSM chiral auxiliary is referred to as a PSM cycle or PSM amidite cycle.

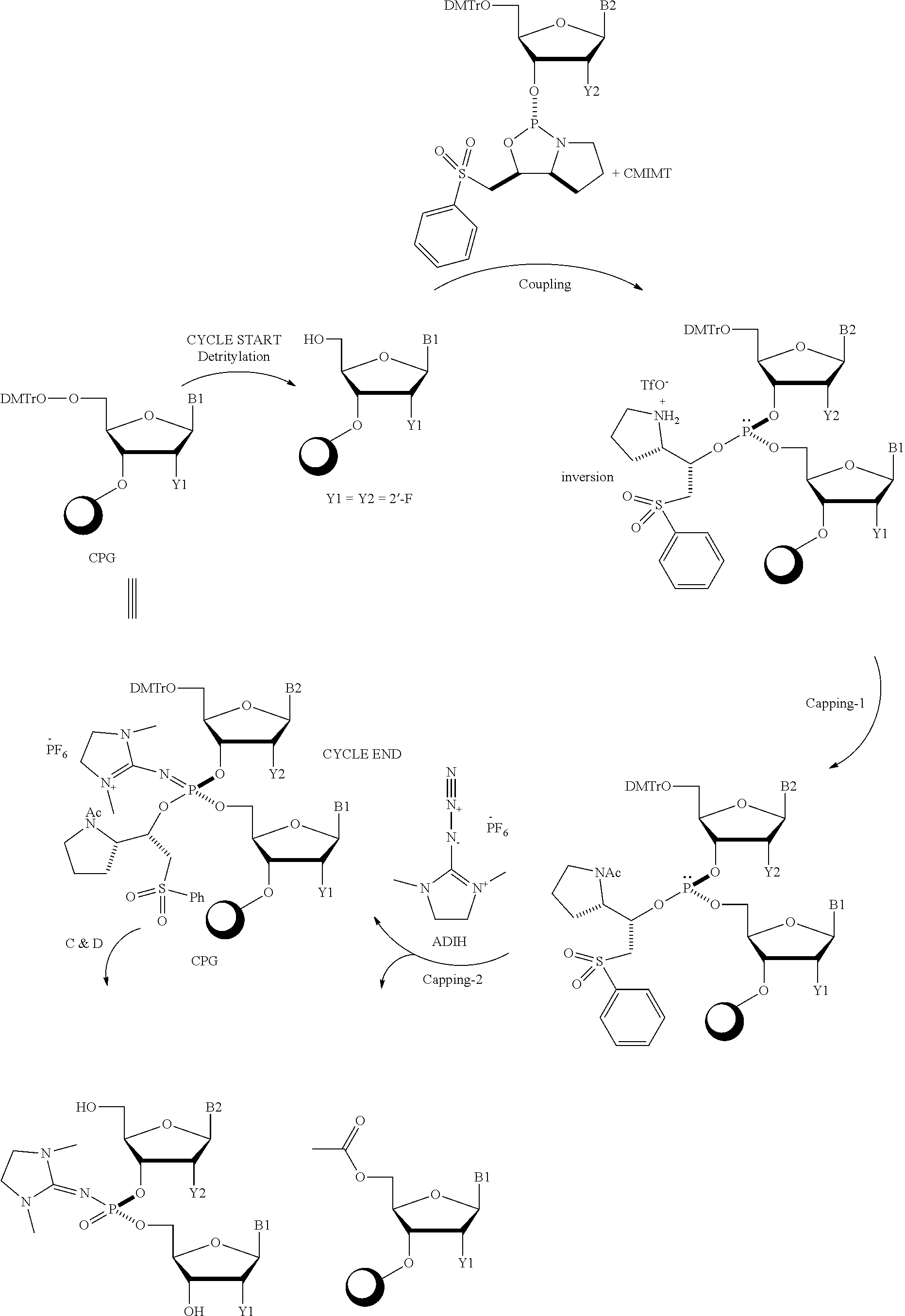

Various cleavage and deprotection methods may be utilized in accordance with the present disclosure. In some embodiments, as appreciated by those skilled in the art, parameters of cleavage and deprotection (e.g., bases, solvents, temperatures, equivalents, time, etc.) can be adjusted in view of, e.g., structures of DMD oligonucleotides to be prepared (e.g., nucleobases, sugars, internucleotidic linkages, and modifications/protections thereof), solid supports, reaction scales, etc. In some embodiments, cleavage and deprotection comprise one, or two or more, individual steps. For example, in some embodiments, a two-step cleavage and deprotection is utilized. In some embodiments, a cleavage and deprotection step comprises a fluoride-containing reagent (e.g., TEA-HF, optionally buffered with additional bases such as TEA) in a suitable solvent (e.g., DMSO/H₂O) at a suitable amount (e.g., about 100 or more (e.g., 100±5) mL/mmol) and is performed at a suitable temperature (e.g., about 0-100, 0-80, 0-50, 0-40, 0-30, 0, 10, 20, 30, 40, 50, 60, 70, 80, 90 or 100° C. (e.g., in one example, 27 t 2° C.)) for a suitable period of time (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more hours (e.g., in one example, 6±0.5 h)). In some embodiments, a cleavage and deprotection step comprises a suitable base (e.g., NR₃) in a suitable solvent (e.g., water) (e.g., conc. NH₄OH) at a suitable amount (e.g., about 200 or more (e.g., 200±5) mL/mmol) and is performed at a suitable temperature (e.g., about 0-100, 0-80, 0-50, 0-40, 0-30, 0, 10, 20, 30, 40, 50, 60, 70, 80, 90 or 100° C. (e.g., in one example, 37 t 2° C.)) for a suitable period of time (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more hours (e.g., in one example, 24±1 h)). In some embodiments, cleavage and deprotection comprises or consists of two steps, wherein one step (e.g., step 1) is 1×TEA-HF in DMSO/H₂O. 100±5 mL/mmol, 27±2° C. and 6±0.5 h, and the other step (e.g., step 2) is conc. NH₄OH, 200±5 mL/mmol, 37±2° C. and 24±1 h. Certain examples of cleavage and deprotection processes are described here.

As appreciated by those skilled in the art, DMD oligonucleotide synthesis is often performed on solid support. Many types of solid support are commercially available and/or can be otherwise prepared/obtained and can be utilized in accordance with the present disclosure. In some embodiments, a solid support is CPG. In some embodiments, a solid support is NittoPhase HL. Types and sizes of solid support can be selected based on desired applications, and in some cases, for a specific use one type of solid support may perform better than the other. In some embodiments, it was observed that for certain preparations CPG can deliver higher crude yields and/or purities compared to certain polymer solid supports such as NittoPhase HL.

Amidites are typically dissolved in solvents at suitable concentrations. In some embodiments, amidites are dissolved in ACN. In some embodiments, amidites are dissolved in a mixture of two or more solvents. In some embodiments, amidites are dissolved in a mixture of ACN and IBN (e.g., 20% ACN/80% IBN). Various concentrations of amidites may be utilized, and may be adjusted in view of specific conditions (e.g., solid support, DMD oligonucleotides to be prepared, reaction times, scales, etc.). In some embodiments, a concentration of about 0.01-0.5, 0.05-0.5, 0.1-0.5, 0.05, 0.1, 0.15, 0.2, 0.25, 0.3, 0.35, 0.4, 0.45 or 0.5 M is utilized. In some embodiments, a concentration of about 0.2 M is utilized. In many embodiments, amidite solutions are dried. In some embodiments, 3 Å molecular sieves are utilized to dry amidite solutions (or keep amidite solutions dry). In some embodiments, molecular sieves are utilized at about 15-20% v/v.

Various equivalents of amidites may be useful for DMD oligonucleotide synthesis. As those skilled in the art will appreciate, equivalents of amidites can be adjusted in view of specific conditions (e.g., solid support, DMD oligonucleotides to be prepared, reaction times, scales, etc.), and the same or different equivalents may be utilized during synthesis. In some embodiments, equivalents of amidites are about 1, 1.5, 2, 2.5, 3, 3.5, 4, 4.5, 5 or more. In some embodiments, a suitable equivalent is about 2. In some embodiments, a suitable equivalent is about 2.5. In some embodiments, a suitable equivalent is about 3. In some embodiments, a suitable equivalent is about 3.5. In some embodiments, a suitable equivalent is about 4.

A number of activators are available in the art and may be utilized in accordance with the present disclosure. In some embodiments, an activator is ETT. In some embodiments, an activator is CMIMT. In some embodiments, CMIMT is utilized for chirally controlled synthesis. As appreciated by those skilled in the art, the same or different activators may be utilized for different amidites, and may be utilized at different amounts. In some embodiments, activators are utilized at about 40-100%, e.g., 40%, 50%, 60%, 70%, 80% or 90% delivery. In some embodiments, a delivery is about 60% (e.g., for ETT). In some embodiments, a delivery is about 70% (e.g., for CMIMT). In some embodiments, molar ratio of activator/amidite is about 1, 2, 3, 4, 5, 6, 7, 8, 9, 10 or more. In some embodiments, a molar ratio is about 3-6. In some embodiments, a molar ratio is about 1. In some embodiments, a molar ratio is about 2. In some embodiments, a molar ratio is about 3. In some embodiments, a molar ratio is about 4. In some embodiments, a molar ratio is about 5. In some embodiments, a molar ratio is about 6. In some embodiments, a molar ratio is about 7. In some embodiments, a molar ratio is about 8. In some embodiments, a molar ratio is about 9. In some embodiments, a molar ratio is about 10. In some embodiments, a molar ratio is about 2-5, 2-4 or 3-4 (e.g., for ETT). In some embodiments, a molar ratio is about 3.7 (e.g., for ETT). In some embodiments, a molar ratio is about 3-8, 4-8, 4-7, 4-6, 5-7, 5-8 or 5-6 (e.g., for CMIMT). In some embodiments, a molar ratio is about 5.8 (e.g., for CMIMT).

As appreciated by those skilled in the art, various suitable flowrates and reaction times may be utilized for DMD oligonucleotide synthesis, and may be adjusted according to DMD oligonucleotides to be prepared, scales, synthetic setups, etc. In some embodiments, a recycle flow rate utilized for synthesis is about 200 cm/h. In some embodiments, a recycle time is about 1-10 minutes. In some embodiments, a recycle time is about 8 minutes. In some embodiments, a recycle time is about 10 minutes.

Many technologies are available to modify P(III) linkages, e.g., after coupling. For example, various methods are available to convert a P(III) linkage to a P(V) P(═O)-type linkage, e.g., via oxidation. In some embodiments, 2/Pyr/H₂O is utilized. Similarly, many methods are available to convert a P(III) linkage to a P(V) P(═S)-type linkage, e.g., via sulfurization. In some embodiments, as illustrated herein, XH is utilized as a thiolation reagent. Technologies for converting P(III) linkages to P(V) P(═N—)-type linkages are also widely available and can be utilized in accordance with the present disclosure. In some embodiments, as illustrated herein ADIH is employed. Suitable reaction parameters are described herein. In some embodiments, ADIH is used at a concentration of about 0.01-0.5, 0.05-0.5, 0.1-0.5, 0.05, 0.1, 0.15, 0.2, 0.25, 0.3, 0.35, 0.4, 0.45 or 0.5 M. In some embodiments, concentration of ADIH is about 0.25 M. In some embodiments, concentration of ADIH is about 0.3 M. In some embodiments, ADIH is utilized at about 1-50, 1-40, 1-30, 1-25, 1-20, 1-10, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45 or 50 or more equivalent. In some embodiments, equivalent of ADIH is about 7.5. In some embodiments, equivalent of ADIH is about 10. In some embodiments, equivalent of ADIH is about 15. In some embodiments, equivalent of ADIH is about 20. In some embodiments, equivalent of ADIH is about 23. In some embodiments, equivalent of ADIH is about 25. In some embodiments, equivalent of ADIH is about 30. In some embodiments, equivalent of ADIH is about 35. In some embodiments, one experiment, ADIH was utilized at 15.2 equivalent, and 15 min contact time. In some embodiments, depending on amidites, concentrations, equivalents, contact times, etc. of reagents, e.g., ADIH, may be adjusted.

Technologies of the present disclosure are suitable for preparation at various scales. In some embodiments, synthesis are performed at hundreds of umol or more. In some embodiments, a scale is about 200 umol. In some embodiments, a scale is about 300 umol. In some embodiments, a scale is about 400 umol. In some embodiments, a scale is about 500 umol. In some embodiments, a scale is about 550 umol. In some embodiments, a scale is about 600 umol. In some embodiments, a scale is about 650 umol. In some embodiments, a scale is about 700 umol. In some embodiments, a scale is about 750 umol. In some embodiments, a scale is about 800 umol. In some embodiments, a scale is about 850 umol. In some embodiments, a scale is about 900 umol. In some embodiments, a scale is about 950 umol. In some embodiments, a scale is about 1, 2, 3, 4, 5, 6, 7, 8, 9, 10, 11, 12, 13, 14, 15, 16, 17, 18, 19, 20, 21, 22, 23, 24, or 25, or more mmol. In some embodiments, a scale is about 1 mmol or more. In some embodiments, a scale is about 2 mmol or more. In some embodiments, a scale is about 5 mmol or more. In some embodiments, a scale is about 10 mmol or more. In some embodiments, a scale is about 15 mmol or more. In some embodiments, a scale is about 20 mmol or more. In some embodiments, a scale is about 25 mmol or more.

In some embodiments, observed yields were 85-90 OD/umol (e.g., 85,000 OD/mmol for a 10.2 mmol synthesis, with 58.4% crude purity (% FLP)).

Technologies of the present disclosure, among other things, can provide various advantages when utilized for preparing DMD oligonucleotides comprising chirally controlled internucleotidic linkages. e.g., those comprising P—N═ wherein P is a linkage phosphorus. For example, as demonstrated herein, technologies of the present disclosure can provide high crude purities and yields (e.g., in many embodiments, about 55-60% full-length product for a 20-mer DMD oligonucleotide) with minimal amount of shorter DMD oligonucleotides (e.g., from incomplete coupling, decomposition, etc.). Such high crude yields and/or purities, among other things, can significantly reduce downstream purification and can significantly reduce production cost and cost of goods, and in some embodiments, greatly facilitate or make possible large scale commercial production, clinical trials and/or commercial sales.

### Example Procedure for Preparing Chirally Controlled DMD Oligonucleotide Compositions—WV-13864.

Described below are example procedures for preparing WV-13864 using controlled pore glass (CPG) low bulk density solid support(e.g., 2′-fC (acetyl) via CNA linker CPG (600 Å LBD)). Useful phosphoramidites include 5′-ODMTr-2′-F-dA(N6-Bz)-(L)-DPSE phosphoramidite, 5′-ODMTr-2′-F-dC(N4-Ac)-(L)-DPSE phosphoramidite, 5′-ODMTr-2′-F-dG(N2-iBu)-(L)-DPSE phosphoramidite, 5′-ODMTr-2′-F-dU-(L)-DPSE phosphoramidite, 5′-ODMTr-2′-OMe-G(N²-iBu)-(L)-DPSE phosphoramidite, 5′-ODMTr-2′-F-dC(N4-Ac)-(L)-PSM phosphoramidite, 5′-ODMTr-2′-F-dG(N2-iBu)-(L)-PSM phosphoramidite, 5′-DMT-2′-OMe-A (Bz)-p-Cyanoethyl phosphoramidite, and 5′-DMT-2′-OMe-C(Ac)-β-Cyanoethyl phosphoramidite.

0.1 M Xanthane hydride solution (XH) was used for thiolation. Neutral PN linkages were formed utilizing 0.3 M of 2-azido-1,3-dimethyl-imidazolinium hexafluorophosphate (ADIH) in acetonitrile. Oxidation solution was 0.04-0.06 M iodine in pyridine/water, 90/10, v/v. Cap A was N-Methylimidazole in acetonitrile, 20/80, v/v. Cap B was acetic anhydride/2,6-Lutidine/Acetonitrile, 20/30/50, v/v/v. Deblocking was performed using 3% dichloroacetic acid in toluene. NH₄OH used was 28-30% concentrated ammonium hydroxide.

### Detritylation.

To initiate the synthesis, the 5′-ODMTr-2′-F-dC(N4-Ac)-CPG solid support was subjected to acid catalyzed removal of the DMTr protecting group from the 5′-hydroxyl by treatment with 3% (DCA) in toluene. The DMTr removal step was usually visualized with strong red or orange color and can be monitored by UV watch command at the wavelength of 436 nm.

DMTr removal can be repeated at the beginning of a synthesis cycle. In every case, following detritylation, the support-bound material was washed with acetonitrile in preparation for the next step of the synthesis.

### Coupling.

Amidites were dissolved either in acetonitrile (ACN) or in 20% isobutyronitrile (IBN)/80% ACN at a concentration of 0.2M without density correction. The solutions were dried over molecular sieves (3 Å) not less than 4 h before use (15-20%, v/v).

| Amidite | Solvent | Concentration | MS3Å |
|---|---|---|---|
| 5′-ODMTr-2′-OMe-A(N6-Bz)-CE | ACN | 0.2M | 15-20%, v/v |
| 5′-ODMTr-2′-OMe-C(N4-Ac)-CE | ACN | 0.2M | 15-20%, v/v |
| 5′-ODMTr-2′-F-dA(N6-Bz)-(L)-DPSE | ACN | 0.2M | 15-20%, v/v |
| 5′-ODMTr-2′-F-dC(N4-Ac)-(L)-DPSE | ACN | 0.2M | 15-20%, v/v |
| 5′-ODMTr-2′-F-dU-(L)-DPSE | 20% IBN/80% ACN | 0.2M | 15-20%, v/v |
| 5′-ODMTr-2′-F-dG(N2-iBu)-(L)-DPSE | ACN | 0.2M | 15-20%, v/v |
| 5′-ODMTr-2′-OMe-G(N2-iBu)-(L)- | 20% IBN/80% ACN | 0.2M | 15-20%, v/v |
| DPSE | | | |
| 5′-ODMTr-2′-F-dC(N4-Ac)-(L)-PSM | ACN | 0.2M | 15-20%, v/v |
| 5′-ODMTr-2′-F-dG(N2-iBu)-(L)-PSM | ACN | 0.2M | 15-20%, v/v |

Dual activators (CMIMT and ET) coupling approach were utilized. Both activators were dissolved in ACN at a concentration of 0.5M. CMIMT has been used for chirally controlled coupling with CMIMT to amidite molar ratio of 5.833/1. ETT was used for the coupling of standard amidites (for natural phosphate linkages) with ETT to amidite molar ratio of 3.752/1. Recycle time for all DPSE and PSM amidites was 10 min except mG-L-DPSE which was 8 min. All standard amidites were coupled for 8 min.

### Cap-1 (Capping-1, First Capping).

Cap B (Ac₂O/2,6-lutidine/MeCN (2:3:5, v/v/v)) was used. In some embodiments, Cap-1 capped secondary amine groups, e.g., on the chiral auxiliaries. In some embodiments, incomplete protection of secondary amines may lead side reaction resulting in a failed coupling or formation of one or more by-products. In some embodiments, Cap-1 may not be an efficient condition for esterification (e.g., a condition less efficient than Cap-2 (the second capping) for capping unreacted 5′-OH).

### Thiolation for DPSE Cycles.

Following Cap-1, phosphite intermediates, P(11I), were modified with sulfurizing reagent. In an example preparation, 1.2 CV (6-7 equivalent) of sulfurizing reagent (0.1 M XH/pyridine-ACN, 1:1, v/v) was delivered through the synthetic column via flow through mode over 6 min contact time to form P(V).

### Azide Reaction for PSM Cycles.

After Cap-1, a suitable reagent (e.g., comprising —N₃ such as ADIH), in ACN was used to form neutral internucleotidic linkages (PN linkages). In an example preparation, 10.3 eq. of 0.25 M ADIH over 10 min contact time for fG-L-PSM and 25.8 eq. of 0.3 M ADIH over 15 min contact time for fC-L-PSM were utilized in the respective cycles.

### Oxidation for Standard Nucleotide Cycles.

Cap-1 step was not necessary for standard amidite cycle. After coupling of a standard amidite onto the solid support, the phosphite intermediate, P(III), was oxidized with 0.05 M of iodine/water/pyridine solution to form P(V). In an example preparation, 3.5 eq. of oxidation solution delivered to the column by a flow through mode over 2 min contact time for efficient oxidation.

### Cap-2 (Capping-2, a Second Capping).

Coupling efficiency on the solid phase DMD oligonucleotide synthesis for each cycle was approx. 97-100% and monitored by, e.g., release of DMTr cation. Residual uncoupled 5′-hydroxyl groups, typically 1-3% by detrit monitoring, on the solid support were blocked with Cap A (20% N-Methylimidazole in acetonitrile (NMI/ACN=20/80, v/v)) and Cap B (20%:30%:50%=Ac₂O:2,6-Lutidine: ACN (v/v/v)) reagents (e.g., 1:1). Both reagents (e.g., 0.4 CV) were delivered to the column by flow through mode over 0.8 min contact time to prevent formation of failure sequences. Uncapped amine groups may also be protected in this step.

As illustrated herein, in some embodiments, a DPSE amidite or DPSE cycle is Detritylation->Coupling ->Cap-1 (Capping-1, first capping) ->Thiolation ->Cap-2 (Capping-1, Post-capping, second capping); in some embodiments, a PSM amidite or PSM cycle is Detritylation ->Coupling ->Cap-1 (Capping-1, first capping) ->Azide reaction ->Cap-2 (Capping-1, Post-capping, second capping); in some embodiments, a standard amidite or standard cycle (traditional, non-chirally controlled) is Detritylation ->Coupling ->Oxidation ->Cap-2 (Capping-1, Post-capping, second capping).

Synthetic cycles were selected and repeated until the desired length was achieved.

Amine wash.

In some embodiments, provided technologies are particularly effective for preparing DMD oligonucleotides comprising internucleotidic linkages that comprise P—N═, wherein P is the linkage phosphorus. In some embodiments, provided technologies comprise contacting a DMD oligonucleotide intermediate with a base. In some embodiments, a contact is performed after desired DMD oligonucleotide lengths have been achieved. In some embodiments, such a contact provides a DMD oligonucleotide comprising internucleotidic linkages that comprise P—N═, wherein P is the linkage phosphorus. In some embodiments, a contact removes a chiral auxiliary (e.g., those with a G² that is connected to the rest of the molecule through a carbon atom, and the carbon atom is connected to at least one electron-withdrawing group (e.g., WV-CA-231, WV-CA-236, WV-CA-240, etc.)). In some embodiments, a contact is performed utilizing a base or a solution of a base which is substantially free of OH or water (anhydrous). In some embodiments, a base is an amine (e.g., N(R)₃). In some embodiments, a base is N. N-diethylamine (DEA). In some embodiments, a base solution is 20% DEA/ACN. In some embodiments, such a contact with a base lowers levels of by-products which, at one or more locations of internucleotidic linkages that comprise P—N═, have instead natural phosphate linkages.

In an example preparation, an on-column amine wash was performed after completion of DMD oligonucleotide nucleotide synthesis cycles, by five column volume of 20% DEA in acetonitrile over 15 min contact time.

In some embodiments, contact with a base may also remove 2-cyanoethyl group used for construction of standard natural phosphate linkage. In some embodiments, contact with a base provide a natural phosphate linkage (e.g., in a salt form in which the cation is the corresponding ammonium salt of the amine base).

### Cleavage and Deprotection.

After contact with a base, DMD oligonucleotides are exposed to further cleavage and deprotection. In an example preparation, auxiliary removal (e.g., DPSE), cleavage & deprotection was a two steps process. In step 1, CPG solid support with DMD oligonucleotides was treated with 1×TEA-HF solution (DMSO:Water:TEA·3HF:TEA=43:8.6:2.8:1=v/v/v/v, 100±5 uL/umol) for 6±0.5 h at 27±2° C. The bulk slurry was then treated with concentrated ammonium hydroxide (28-30%, 200±10 mL/mmol) for 24±1 h at 37±2° C. (step 2) to release DMD oligonucleotide from the solid support. Crude product was collected by filtration. Filtrates were combined with washes (e.g., water) of the solid support. In some embodiments, observed yields were about 80-90 OD/umole.

Among other things, provided technologies provided high crude purities and/or yields. In many preparations (various scales, reagents concentrations, reaction times, etc.), about 55-60% crude purities (% FLP) were obtained, with minimal amount of shorter DMD oligonucleotides (e.g., from incomplete coupling, decomposition, side-reactions, etc.). In many embodiments, amounts of the most significant shorter DMD oligonucleotide is no more than about 2-10%, often no more than 2-4% (e.g., in some embodiments, as low as about 2% (the most significant shorter DMD oligonucleotide being N-3)).

Various technologies are available for DMD oligonucleotide purification and can be utilized in accordance with the present disclosure. In some embodiments, crude products were further purified (e.g., over 90% purity) using, e.g., AEX purification, and/or UF/DF.

Using technologies described herein, various DMD oligonucleotides comprising diverse base sequences, modifications (e.g., nucleobase, sugar, and internucleotidic linkage modifications) and/or patterns thereof, linkage phosphorus stereochemistry and/or patterns thereof, etc. were prepared at various scales from umol to mmol. Such DMD oligonucleotides have various targets and may function through various mechanisms. Certain such DMD oligonucleotides were presented in the Tables of the present disclosure.

As appreciated by those skilled in the art, examples described herein are for illustration only. Those skilled in the art will appreciate that various conditions, parameters, etc. may be adjusted according to, e.g., instrumentation, scales, reagents, reactants, desired outcomes, etc. Certain results may be further improved using various technologies in accordance with the present disclosure. Among other things, provided DMD oligonucleotides and compositions thereof can provide significantly improved properties and/or skipping of exon 51, e.g., in various assays and in vivo models, and may be particularly useful for preventing and/or treating various conditions, disorders or diseases. Certain data are provided in Examples herein.

### Example & Timelines for ‘Pre-Differentiation’ of Patient Myoblasts for Gymnotic Dosing

Various technologies, e.g., those described in U.S. Pat. Nos. 9,394,333, 9,744,183, 9,605,019, 9,598,458, US 2015/0211006, US 2017/0037399, WO 2017/015555, WO 2017/192664, WO 2017/015575, WO2017/062862, WO 2017/160741, WO 2017/192679, and WO 2017/210647, etc., can be utilized in accordance with the present disclosure to assess properties and/or activities of technologies of the present disclosure. In some embodiments, technologies of the present disclosure, e.g., DMD oligonucleotides and compositions and methods of use thereof, demonstrate unexpectedly superior results compared to a suitable reference technology (e.g., a technology based on a stereorandom composition of DMD oligonucleotides having the same base sequence but no neutral and/or cationic internucleotidic linkages at physiological pH). Described below are example technologies that can be useful for assessing properties and/or activities of DMD oligonucleotides described in the present disclosure. Those skilled in the art understand that conditions illustrated below may be varied/modified, and additionally and/or alternatively, other suitable reagents, temperatures, conditions, time periods, amounts, etc., may be utilized in accordance with the present disclosure.

Unless otherwise noted, in various experiments, cells and animals used in experiments were used in conditions typical for those cells or animals. Unless otherwise noted, in in vitro experiments, various cells were grown under standard conditions (e.g., the most common conditions used for a particular cell type, cell line or a similar cell type or line), e.g., with ordinary growth medium, normal temp (37 C), and gravity and atmospheric pressure typical of Cambridge. MA. Animals were kept under standard laboratory conditions, generally at room temperature, or a few degrees cooler, with normal conditions of feeding, cage size, etc. Neither cells nor animals, unless otherwise described, were subjected to extremes of temperature (e.g., cold shock or heat shock), pressure, gravity, ambient sound, food or nutrient deprivation, etc.

### Maintenance of Patient Derived Myoblast Cell Lines:

DMD Δ52 and DMD Δ45-52 myoblast cells were maintained in complete Skeletal Muscle Growth Medium (Promocell, Heidelberg, Germany) supplemented with 5% FBS, IX Penicillin-Streptomycin and 1× L-Glutamine. Flasks or plates were coated with Matrigel:DMEM solution (1:100) for a suitable period of time, e.g., 30 mins, after which Matrigel:DMEM solution was removed via aspiration before seeding of cells in complete Skeletal Muscle Growth Medium.

### Standard Dosing Procedure (0 Das Pre-Differentiation)

On Day 1: Coat suitable cell growth containers, e.g., 6-well plates or 24-well plates, with Matrigel: DMEM Solution. Incubate at a condition, e.g., 37° C., 5% CO₂ for a suitable period of time, e.g., 30 mins. Aspirate, and seed a suitable number of cells to cell growth containers, e.g., 150K cells/well in a total of 1500 μl of complete growth medium in 6-well plate, and 30K cells/well in 500 ul of growth medium in a 24-well plate. Incubate at a suitable condition for a suitable period of time, e.g., 37° C., 5% CO₂ overnight.

On Day 2: Prepare a suitable Differentiation medium, e.g., DMEM+5% Horse Serum+10 μg/ml Insulin. Prepare suitable DMD oligonucleotide dilutions in Differentiation Medium, e.g., serial dilutions of 30 uM, 10 uM, 3.33 uM, 1.11 uM, 0.37 uM. Aspirate growth medium off of adherent cells, and add DMD oligonucleotide:Differentiation Medium solution to cells. Oligonucleotides remain on cells (no media change) until cell harvesting.

On Day 6: Obtain RNA. In a typical procedure, a suitable number of cells, e.g., cells from wells of a 24-well plate, were washed, e.g., with cold PBS, followed by addition of a suitable amount of a reagent for RNA extraction and storage of sample/RNA extraction, e.g., 500 ul/well TRIZOL in 24-well plate and freezing plate at −80° C. or continuing with RNA extraction to obtain RNA.

On Day 8: Obtain protein. In a typical procedure, a suitable number of cells, e.g., cells in wells of 6-well plate, were washe, e.g., with cold PBS. A suitable amount of a suitable lysis buffer was then added—e.g., in a typical procedure, 200 ul/well of RIPA supplemented with protease inhibitors for a 6-well plate. After lysis the sample can be stored, e.g., freezing at −80° C., or continue with protein extraction.

Other suitable procedures may be employed, for example, those described below. As appreciated by those skilled in the art, many parameters, such as reagents, temperatures, conditions, time periods, amounts, etc., may be modified.

### 4 Days Pre-Differentiation Dosing Procedure

On Day 1: Coat 6-well plates or 24-well plates with Matrigel: DMEM Solution. Incubate at 37° C., 5% CO₂ for 30 mins. Aspirate, seed 150K cells/well in a total of 1500 μl of complete growth medium in 6-well plate, and 30K cells/well in 500 ul of growth medium in a 24-well plate. Incubate at 37° C., 5% CO₂ overnight.

On Day 2: Prepare Differentiation medium as follows: DMEM+5% Horse Serum+10 μg/ml Insulin. Aspirate Growth Media and replace with Differentiation Media.

On Day 6: Cells have differentiated for 4 days. Prepare DMD oligonucleotide dilutions in Differentiation Medium, for example serial dilutions of 30 uM, 10 uM, 3.33 uM, 1.11 uM, 0.37 uM. Aspirate Differentiation medium off of adherent cells, and add DMD oligonucleotide:Differentiation Medium solution to cells. Oligonucleotides remain on cells (no media change) until cell harvesting.

On Day 10: Wash cells in 24-well plate with cold PBS, add 500 ul/well TRIZOL in 24-well plate and freeze plate at −80° C. or continue with RNA extraction.

On Day 12: Wash cells in 6-well plate with cold PBS. Add 200 ul/well of RIPA supplemented with protease inhibitors. Freeze plate at −80° C. or continue with protein extraction.

7 days Pre-Differentiation Dosing Procedure

On Day 1: Coat 6-well plates or 24-well plates with Matrigel: DMEM Solution. Incubate at 37° C., 5% CO₂ for 30 mins. Aspirate, seed 150K cells/well in a total of 1500 μl of complete growth medium in 6-well plate, and 30K cells/well in 500 ul of growth medium in a 24-well plate. Incubate at 37° C., 5% CO₂ overnight.

On Day 2: Prepare Differentiation medium as follows: DMEM+5% Horse Serum+10 μg/ml Insulin. Aspirate Growth Media and replace with Differentiation Media.

On Day 9: Cells have differentiated for 7 days. Prepare DMD oligonucleotide dilutions in Differentiation Medium, for example serial dilutions of 30 uM, 10 uM, 3.33 uM, 1.11 uM, 0.37 uM. Aspirate Differentiation medium off of adherent cells, and add DMD oligonucleotide:Differentiation Medium solution to cells. Oligonucleotides remain on cells (no media change) until cell harvesting.

On Day 13: Wash cells in 24-well plate with cold PBS, add 500 ul/well TRIZOL in 24-well plate and freeze plate at −80° C. or continue with RNA extraction.

On Day 15: Wash cells in 6-well plate with cold PBS. Add 200 ul/well of RIPA supplemented with protease inhibitors. Freeze plate at −80° C. or continue with protein extraction.

### 10 Das Pre-Differentiation Dosing Procedure

On Day 1: Coat 6-well plates or 24-well plates with Matrigel: DMEM Solution. Incubate at 37° C., 5% CO₂ for 30 mins. Aspirate, seed 150K cells/well in a total of 1500 μl of complete growth medium in 6-well plate, and 30K cells/well in 500 ul of growth medium in a 24-well plate. Incubate at 37° C., 5% CO₂ overnight.

On Day 2: Prepare Differentiation medium as follows: DMEM+5% Horse Serum+10 μg/ml Insulin. Aspirate Growth Media and replace with Differentiation Media.

On Day 12: Cells have differentiated for 10 days. Prepare DMD oligonucleotide dilutions in Differentiation Medium, for example serial dilutions of 30 uM, 10 uM, 3.33 uM, 1.11 uM, 0.37 uM. Aspirate Differentiation medium off of adherent cells, and add DMD oligonucleotide:Differentiation Medium solution to cells. Oligonucleotides remain on cells (no media change) until cell harvesting.

On Day 16: Wash cells in 24-well plate with cold PBS, add 500 ul/well TRIZOL in 24-well plate and freeze plate at −80° C. or continue with RNA extraction.

On Day 18: Wash cells in 6-well plate with cold PBS. Add 200 ul/well of RIPA supplemented with protease inhibitors. Freeze plate at −80° C. or continue with protein extraction.

### Example 9. Provided Oligonucleotides and Oligonucleotide Compositions can Provide Functional DMD Proteins

DMDdelta48-50 myoblasts were cultured in differentiation media with oligonucleotide under free uptake conditions for six or seven days. Differentiated myotubes were washed with PBS and lysed in 75 μl of RIPA lysis buffer supplemented with protease inhibitor. Total protein concentration was determined by dilution in Pierce Protein 660 nm Assay Reagent, by comparison to a standard curve of bovine serum albumin.

Dystrophin quantitation was performed on a WES™ instrument, according to the manufacturer's instructions using a 66 kDa-440 kDa Separation Module, the Anti-Rabbit Detection Module and the Anti-Mouse Detection Module depending on the primary antibody used. Samples were diluted to 0.5 ug/μl in 0.1× Sample Buffer (10× Sample Buffer from the Separation Module), mixed with Fluorescent Master Mix (from the Separation Module) and denatured at 95° C. for 5 mins. A calibration curve was generated using lysate from immortalized healthy human myotubes (“wild type”) diluted into lysate from mock-treated DMD myotubes. The samples, blocking reagent (antibody diluent), primary antibodies (1:50 Anti-Dystrophin [Abcam], 1:1000 Anti-Vinculin [Thermo] in Protein Simple Antibody Diluent), Horseradish peroxide (HRP)-conjugated secondary antibodies (ready to use anti-mouse diluted 1:10 in ready to use anti-rabbit) plus chemiluminescent substrate were pipetted into the plate (part of Separation Module). Instrument default settings were used: stacking and separation at 475 V for 30 min; blocking reagent for 5 min, primary and secondary antibody both for 30 min; Luminol/peroxide chemiluminescence detection for about 15 min (exposures of 1, 2, 4, 8, 16, 32, 64, 128, 512 see). The chemiluminescence produced was automatically quantified by area under the curve (AUC) of detected peaks by the Compass software and displayed as an electropherogram or as a virtual blot-like image. Standard curves were generated by plotting % standard versus the ratio of (AUC of dystrophin)/(AUC of vinculin). The calculated concentrations of the samples were interpolated from this standard curve (Table 9).

TABLE 9
Certain protein production data.

| | | WV- | WV- | WV- | WV- | WV- | WV- | WV- | WV- | WV- | WV- | WV- | WV- | WV- | WV- | WV- |
|---|---|---|---|---|---|---|---|---|---|---|---|---|---|---|---|---|
| | water | 3152 | 31562 | 31565 | 31578 | 31568 | 31580 | 31561 | 31573 | 31563 | 31564 | 31576 | 31567 | 31569 | 31582 | 31583 |
| Bio rep 1 | 0.1 | 1 | 1.3 | 2.8 | 2.6 | 3.3 | 3 | 2.5 | 2.4 | 1.5 | 2.7 | 2.3 | 1.5 | 1.8 | 3.3 | 1.9 |
| Bio rep 2 | 0.1 | 1 | 1.2 | 2.2 | 2.6 | 2.6 | 2.5 | | 2.4 | 1.7 | 2.9 | 5.9 | 2.7 | 0.2 | 7.2 | 2.1 |

### Example 10. In Silico Sequence Design

An in silico analysis was performed to determine sequence complementarity between oligonucleotides (e.g., of 20 nucleoside in length) of interest and sequences within DNA or RNA of three key species: Homo sapiens (human), Mus musculus (mouse), and Macaca fascicularis (monkey). Multiple software/programs can be utilized for sequence alignment in accordance with the present disclosure. In some embodiments, sequence searches were performed with the bowtie and blastn software programs. Complementary ‘hits’ were identified and recorded if they contained two or fewer mismatches, of, e.g., a 20 nucleotide length.

In some embodiments, in silico design was used to determine complementarity between oligonucleotides and mouse and monkey Dystrophin transcript. A perfect match between oligonucleotides and both human and animal (e.g., monkey) Dystrophin transcripts, in some embodiments, enable studies, e.g., toxicology studies, using the same oligonucleotides and oligonucleotide compositions in various species. While not wishing to be bound by theory, in some embodiments, a non-perfect match (e.g., of 18 or 19 nucleobases of a 20-nucleobase long oligonucleotide) may result in less efficient target engagement. Table 10 showed certain examples of oligonucleotides with complementarity between human, mouse and monkey Dystrophin transcripts.

TABLE 10
Oligonucleotides and sequences with
complementarity among three species
(human, mouse, monkey).

| | SEQ | |
|---|---|---|
| | ID | |
| Oligonucleotide | NO | Base Sequence |
| WV-20011 | 424 | GGUAAGUUCUGUCCAAGCCC |
| WV-20052 | 425 | GUACCUCCAACAUCAAGGAA |
| WV-20059 | 426 | CAACAUCAAGGAAGAUGGCA |
| WV-20073 | 427 | AUGGCAUUUCUAGUUUGGAG |
| WV-20074 | 428 | UGGCAUUUCUAGUUUGGAGA |
| WV-20075 | 429 | GGCAUUUCUAGUUUGGAGAU |
| WV-20076 | 430 | GCAUUUCUAGUUUGGAGAUG |
| WV-20094 | 431 | UGGCAGUUUCCUUAGUAACC |
| WV-20097 | 432 | CAGUUUCCUUAGUAACCACA |
| WV-20098 | 433 | AGUUUCCUUAGUAACCACAG |
| WV-20101 | 434 | UUCCUUAGUAACCACAGGUU |
| WV-20119 | 435 | UUGUGUCACCAGAGUAACAG |

In some embodiments, the present disclosure provides oligonucleotides whose base sequences are or comprise a base sequence in Table 10.

In some embodiments, in silico design was used to determine complementarity between oligonucleotides and mouse and murine Dystrophin transcripts. Because mice are amenable to genetic manipulation, it is feasible that a murine model of muscular dystrophy could be developed by deleting exons from murine Dystrophin gene. In some embodiments, a complete match to mouse dystrophin may enable studies in a Dystrophin internal-deletion mouse model. Among other things, data from mouse models, e.g., data of exon skipping for reading frame restoration and/or dystrophin protein production/restoration, in vivo in a mouse model can be useful for development of therapeutic agents for humans.

### EQUIVALENTS

Having described some illustrative embodiments of the disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the disclosure. In particular, although many of the examples presented herein involve specific combinations of method acts or system elements, it should be understood that those acts and those elements may be combined in other ways to accomplish the same objectives. Acts, elements, and features discussed only in connection with one embodiment are not intended to be excluded from a similar role in other embodiments. Further, for the one or more means-plus-function limitations, if any, recited in the following claims, the means are not intended to be limited to the means disclosed herein for performing the recited function, but are intended to cover in scope any means, known now or later developed, for performing the recited function.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

The foregoing written specification is considered to be sufficient to enable one skilled in the art to practice the invention. The present disclosure is not to be limited in scope by examples provided. Examples are intended as illustration of one or more aspect of an invention and other functionally equivalent embodiments are within the scope of the invention. Various modifications in addition to those shown and described herein will become apparent to those skilled in the art from the foregoing description and fall within the scope of the appended claims. Advantages and objects of the invention are not necessarily encompassed by each embodiment of the invention.

1-59. (canceled)

60. An oligonucleotide, wherein the oligonucleotide is:
fG*SfU*SfAn001RfC*SfC*SfUn001RfC*SfC*SmAfA*SmC*SfA*SmUfC*SfA*SfA*SfGn001RfG*SfA*SfA (SEQ ID NO: 172) or a salt thereof, wherein:
f represents a 2′-F modified nucleoside;
m represents a 2′-OMe modified nucleoside;
*S represents a Sp phosphorothioate; and
n001R is 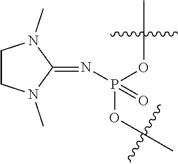 [SMILES: *OP(=O)(N=C1N(C)CCN1C)O*]
wherein the phosphorus is of the Rp configuration.

61. The oligonucleotide of claim 60, wherein the oligonucleotide is a pharmaceutically acceptable salt.

62. The oligonucleotide of claim 60, wherein the oligonucleotide is a sodium salt.

63. The oligonucleotide of claim 60, wherein each chiral internucleotidic linkage of the oligonucleotide independently has a diastereopurity of at least 90%.

64. An oligonucleotide, wherein the oligonucleotide is:
fA*SfU*SfGn001RfG*SfC*SfAn001RfU*SfU*SmUfC*SmU*SfA*SmGfU*SfU*SfA*SfGn001RfG*SfA*SfG (SEQ ID NO: 174) or a salt thereof, wherein:
f represents a 2′-F modified nucleoside;
m represents a 2′-OMe modified nucleoside;
*S represents a Sp phosphorothioate; and
n001R is 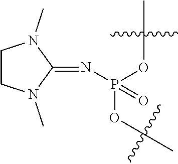 [SMILES: *OP(=O)(N=C1N(C)CCN1C)O*]
wherein the phosphorus is of the Rp configuration.

65. The oligonucleotide of claim 64, wherein the oligonucleotide is a pharmaceutically acceptable salt.

66. The oligonucleotide of claim 64, wherein the oligonucleotide is a sodium salt.

67. The oligonucleotide of claim 64, wherein each chiral internucleotidic linkage of the oligonucleotide independently has a diastereopurity of at least 90%.

68. An oligonucleotide, wherein the oligonucleotide is:
fU*SfG*SfSGn001RfC*SfA*SfGn001RfU*SfU*SmUfC*SmC*SfU*SmUfA*SfG*SfU*SfAn001RfA*SfC*SfC (SEQ ID NO: 178) or a salt thereof, wherein:
f represents a 2′-F modified nucleoside;
m represents a 2′-OMe modified nucleoside;
*S represents a Sp phosphorothioate; and
n001R is 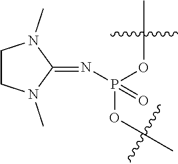 [SMILES: *OP(=O)(N=C1N(C)CCN1C)O*]
wherein the phosphorus is of the Rp configuration.

69. The oligonucleotide of claim 68, wherein the oligonucleotide is a pharmaceutically acceptable salt.

70. The oligonucleotide of claim 68, wherein the oligonucleotide is a sodium salt.

71. The oligonucleotide of claim 68, wherein each chiral internucleotidic linkage of the oligonucleotide independently has a diastereopurity of at least 90%.


SEQUENCE LISTING (440 sequences)

SEQ ID NO: 1 - RNA, 20 nt, synthetic construct
  misc_feature 1..20: note = Synthetic
  source 1..20: mol_type = other RNA
tcaaggaagatggcatttct

SEQ ID NO: 2 - RNA, 20 nt, synthetic construct
  misc_feature 1..20: note = Synthetic
  source 1..20: mol_type = other RNA
tcaaggaagatggcatttct

SEQ ID NO: 3 - RNA, 20 nt, synthetic construct
  misc_feature 1..20: note = Synthetic
  source 1..20: mol_type = other RNA
ctccggttctgaaggtgttc

SEQ ID NO: 4 - DNA, 25 bp, synthetic construct
  misc_feature 1..25: note = Synthetic
  source 1..25: mol_type = other DNA
gttgcctccggttctgaaggtgttc

SEQ ID NO: 5 - DNA, 20 bp, synthetic construct
  misc_feature 1..20: note = Synthetic
  source 1..20: mol_type = other DNA
ctccggttctgaaggtgttc

SEQ ID NO: 6 - DNA, 28 bp, synthetic construct
  misc_feature 1..28: note = Synthetic
  source 1..28: mol_type = other DNA
tgcctccggttctgaaggtgttcttgta

SEQ ID NO: 7 - RNA, 20 nt, synthetic construct
  misc_feature 1..20: note = Synthetic
  source 1..20: mol_type = other RNA
tccggttctgaaggtgttct

SEQ ID NO: 8 - RNA, 20 nt, synthetic construct
  misc_feature 1..20: note = Synthetic
  source 1..20: mol_type = other RNA
ctccggttctgaaggtgttc

SEQ ID NO: 9 - RNA, 20 nt, synthetic construct
  misc_feature 1..20: note = Synthetic
  source 1..20: mol_type = other RNA
ctccggttctgaaggtgttc

SEQ ID NO: 10 - RNA, 20 nt, synthetic construct
  misc_feature 1..20: note = Synthetic
  source 1..20: mol_type = other RNA
tcaaggaagatggcatttct

SEQ ID NO: 11 - RNA, 20 nt, synthetic construct
  misc_feature 1..20: note = Synthetic
  source 1..20: mol_type = other RNA
tcaaggaagatggcatttct

SEQ ID NO: 12 - RNA, 20 nt, synthetic construct
  misc_feature 1..20: note = Synthetic
  source 1..20: mol_type = other RNA
tccggttctgaaggtgttct

SEQ ID NO: 13 - RNA, 20 nt, synthetic construct
  misc_feature 1..20: note = Synthetic
  source 1..20: mol_type = other RNA
tcaaggaagatggcatttct

SEQ ID NO: 14 - RNA, 20 nt, synthetic construct
  misc_feature 1..20: note = Synthetic
  source 1..20: mol_type = other RNA
tcaaggaagatggcatttct

SEQ ID NO: 15 - RNA, 20 nt, synthetic construct
  misc_feature 1..20: note = Synthetic
  source 1..20: mol_type = other RNA
tcaaggaagatggcatttct

SEQ ID NO: 16 - RNA, 20 nt, synthetic construct
  misc_feature 1..20: note = Synthetic
  source 1..20: mol_type = other RNA
tcaaggaagatggcatttct

SEQ ID NO: 17 - RNA, 20 nt, synthetic construct
  misc_feature 1..20: note = Synthetic
  source 1..20: mol_type = other RNA
tcaaggaagatggcatttct

SEQ ID NO: 18 - RNA, 20 nt, synthetic construct
  misc_feature 1..20: note = Synthetic
  source 1..20: mol_type = other RNA
tcaaggaagatggcatttct

SEQ ID NO: 19 - RNA, 20 nt, synthetic construct
  misc_feature 1..20: note = Synthetic
  source 1..20: mol_type = other RNA
tcaaggaagatggcatttct

SEQ ID NO: 20 - RNA, 20 nt, synthetic construct
  misc_feature 1..20: note = Synthetic
  source 1..20: mol_type = other RNA
tcaaggaagatggcatttct

SEQ ID NO: 21 - RNA, 20 nt, synthetic construct
  misc_feature 1..20: note = Synthetic
  source 1..20: mol_type = other RNA
tcaaggaagatggcatttct

SEQ ID NO: 22 - RNA, 20 nt, synthetic construct
  misc_feature 1..20: note = Synthetic
  source 1..20: mol_type = other RNA
tcaaggaagatggcatttct

SEQ ID NO: 23 - RNA, 20 nt, synthetic construct
  misc_feature 1..20: note = Synthetic
  source 1..20: mol_type = other RNA
tcaaggaagatggcatttct

SEQ ID NO: 24 - RNA, 20 nt, synthetic construct
  misc_feature 1..20: note = Synthetic
  source 1..20: mol_type = other RNA
tgaaatctgccagagcaggt

SEQ ID NO: 25 - RNA, 20 nt, synthetic construct
  misc_feature 1..20: note = Synthetic
  source 1..20: mol_type = other RNA
tgaaatctgccagagcaggt

SEQ ID NO: 26 - RNA, 20 nt, synthetic construct
  misc_feature 1..20: note = Synthetic
  source 1..20: mol_type = other RNA
aatctgccagagcaggtacc

SEQ ID NO: 27 - RNA, 20 nt, synthetic construct
  misc_feature 1..20: note = Synthetic
  source 1..20: mol_type = other RNA
ctgccagagcaggtacctcc

SEQ ID NO: 28 - RNA, 20 nt, synthetic construct
  misc_feature 1..20: note = Synthetic
  source 1..20: mol_type = other RNA
ccagagcaggtacctccaac

SEQ ID NO: 29 - RNA, 20 nt, synthetic construct
  misc_feature 1..20: note = Synthetic
  source 1..20: mol_type = other RNA
gagcaggtacctccaacatc

SEQ ID NO: 30 - RNA, 20 nt, synthetic construct
  misc_feature 1..20: note = Synthetic
  source 1..20: mol_type = other RNA
caggtacctccaacatcaag

SEQ ID NO: 31 - RNA, 20 nt, synthetic construct
  misc_feature 1..20: note = Synthetic
  source 1..20: mol_type = other RNA
aggtacctccaacatcaagg

SEQ ID NO: 32 - RNA, 20 nt, synthetic construct
  misc_feature 1..20: note = Synthetic
  source 1..20: mol_type = other RNA
ggtacctccaacatcaagga

SEQ ID NO: 33 - RNA, 20 nt, synthetic construct
  misc_feature 1..20: note = Synthetic
  source 1..20: mol_type = other RNA
gtacctccaacatcaaggaa

SEQ ID NO: 34 - RNA, 20 nt, synthetic construct
  misc_feature 1..20: note = Synthetic
  source 1..20: mol_type = other RNA
tacctccaacatcaaggaag

SEQ ID NO: 35 - RNA, 20 nt, synthetic construct
  misc_feature 1..20: note = Synthetic
  source 1..20: mol_type = other RNA
acctccaacatcaaggaaga

SEQ ID NO: 36 - RNA, 20 nt, synthetic construct
  misc_feature 1..20: note = Synthetic
  source 1..20: mol_type = other RNA
cctccaacatcaaggaagat

SEQ ID NO: 37 - RNA, 20 nt, synthetic construct
  misc_feature 1..20: note = Synthetic
  source 1..20: mol_type = other RNA
ctccaacatcaaggaagatg

SEQ ID NO: 38 - RNA, 20 nt, synthetic construct
  misc_feature 1..20: note = Synthetic
  source 1..20: mol_type = other RNA
tccaacatcaaggaagatgg

SEQ ID NO: 39 - RNA, 20 nt, synthetic construct
  misc_feature 1..20: note = Synthetic
  source 1..20: mol_type = other RNA
ccaacatcaaggaagatggc

SEQ ID NO: 40 - RNA, 20 nt, synthetic construct
  misc_feature 1..20: note = Synthetic
  source 1..20: mol_type = other RNA
caacatcaaggaagatggca

SEQ ID NO: 41 - RNA, 20 nt, synthetic construct
  misc_feature 1..20: note = Synthetic
  source 1..20: mol_type = other RNA
aacatcaaggaagatggcat

SEQ ID NO: 42 - RNA, 20 nt, synthetic construct
  misc_feature 1..20: note = Synthetic
  source 1..20: mol_type = other RNA
acatcaaggaagatggcatt

SEQ ID NO: 43 - RNA, 20 nt, synthetic construct
  misc_feature 1..20: note = Synthetic
  source 1..20: mol_type = other RNA
catcaaggaagatggcattt

SEQ ID NO: 44 - RNA, 20 nt, synthetic construct
  misc_feature 1..20: note = Synthetic
  source 1..20: mol_type = other RNA
atcaaggaagatggcatttc

SEQ ID NO: 45 - RNA, 20 nt, synthetic construct
  misc_feature 1..20: note = Synthetic
  source 1..20: mol_type = other RNA
tcaaggaagatggcatttct

SEQ ID NO: 46 - RNA, 20 nt, synthetic construct
  misc_feature 1..20: note = Synthetic
  source 1..20: mol_type = other RNA
tcaaggaagatggcatttct

SEQ ID NO: 47 - RNA, 20 nt, synthetic construct
  misc_feature 1..20: note = Synthetic
  source 1..20: mol_type = other RNA
caaggaagatggcatttcta

SEQ ID NO: 48 - RNA, 20 nt, synthetic construct
  misc_feature 1..20: note = Synthetic
  source 1..20: mol_type = other RNA
aaggaagatggcatttctag

SEQ ID NO: 49 - RNA, 20 nt, synthetic construct
  misc_feature 1..20: note = Synthetic
  source 1..20: mol_type = other RNA
aggaagatggcatttctagt

SEQ ID NO: 50 - RNA, 20 nt, synthetic construct
  misc_feature 1..20: note = Synthetic
  source 1..20: mol_type = other RNA
ggaagatggcatttctagtt

SEQ ID NO: 51 - RNA, 20 nt, synthetic construct
  misc_feature 1..20: note = Synthetic
  source 1..20: mol_type = other RNA
gaagatggcatttctagttt

SEQ ID NO: 52 - RNA, 20 nt, synthetic construct
  misc_feature 1..20: note = Synthetic
  source 1..20: mol_type = other RNA
aagatggcatttctagtttg

SEQ ID NO: 53 - RNA, 20 nt, synthetic construct
  misc_feature 1..20: note = Synthetic
  source 1..20: mol_type = other RNA
aagatggcatttctagtttg

SEQ ID NO: 54 - RNA, 20 nt, synthetic construct
  misc_feature 1..20: note = Synthetic
  source 1..20: mol_type = other RNA
agatggcatttctagtttgg

SEQ ID NO: 55 - RNA, 20 nt, synthetic construct
  misc_feature 1..20: note = Synthetic
  source 1..20: mol_type = other RNA
gatggcatttctagtttgga

SEQ ID NO: 56 - RNA, 20 nt, synthetic construct
  misc_feature 1..20: note = Synthetic
  source 1..20: mol_type = other RNA
atggcatttctagtttggag

SEQ ID NO: 57 - RNA, 20 nt, synthetic construct
  misc_feature 1..20: note = Synthetic
  source 1..20: mol_type = other RNA
atggcatttctagtttggag

SEQ ID NO: 58 - RNA, 20 nt, synthetic construct
  misc_feature 1..20: note = Synthetic
  source 1..20: mol_type = other RNA
tggcatttctagtttggaga

SEQ ID NO: 59 - RNA, 20 nt, synthetic construct
  misc_feature 1..20: note = Synthetic
  source 1..20: mol_type = other RNA
ggcatttctagtttggagat

SEQ ID NO: 60 - RNA, 20 nt, synthetic construct
  misc_feature 1..20: note = Synthetic
  source 1..20: mol_type = other RNA
gcatttctagtttggagatg

SEQ ID NO: 61 - RNA, 20 nt, synthetic construct
  misc_feature 1..20: note = Synthetic
  source 1..20: mol_type = other RNA
gcatttctagtttggagatg

SEQ ID NO: 62 - RNA, 20 nt, synthetic construct
  misc_feature 1..20: note = Synthetic
  source 1..20: mol_type = other RNA
catttctagtttggagatgg

SEQ ID NO: 63 - RNA, 20 nt, synthetic construct
  misc_feature 1..20: note = Synthetic
  source 1..20: mol_type = other RNA
atttctagtttggagatggc

SEQ ID NO: 64 - RNA, 20 nt, synthetic construct
  misc_feature 1..20: note = Synthetic
  source 1..20: mol_type = other RNA
tttctagtttggagatggca

SEQ ID NO: 65 - RNA, 20 nt, synthetic construct
  misc_feature 1..20: note = Synthetic
  source 1..20: mol_type = other RNA
ttctagtttggagatggcag

SEQ ID NO: 66 - RNA, 20 nt, synthetic construct
  misc_feature 1..20: note = Synthetic
  source 1..20: mol_type = other RNA
tctagtttggagatggcagt

SEQ ID NO: 67 - RNA, 20 nt, synthetic construct
  misc_feature 1..20: note = Synthetic
  source 1..20: mol_type = other RNA
ctagtttggagatggcagtt

SEQ ID NO: 68 - RNA, 20 nt, synthetic construct
  misc_feature 1..20: note = Synthetic
  source 1..20: mol_type = other RNA
tagtttggagatggcagttt

SEQ ID NO: 69 - RNA, 20 nt, synthetic construct
  misc_feature 1..20: note = Synthetic
  source 1..20: mol_type = other RNA
agtttggagatggcagtttc

SEQ ID NO: 70 - RNA, 20 nt, synthetic construct
  misc_feature 1..20: note = Synthetic
  source 1..20: mol_type = other RNA
gtttggagatggcagtttcc

SEQ ID NO: 71 - RNA, 20 nt, synthetic construct
  misc_feature 1..20: note = Synthetic
  source 1..20: mol_type = other RNA
tttggagatggcagtttcct

SEQ ID NO: 72 - RNA, 20 nt, synthetic construct
  misc_feature 1..20: note = Synthetic
  source 1..20: mol_type = other RNA
ttggagatggcagtttcctt

SEQ ID NO: 73 - RNA, 20 nt, synthetic construct
  misc_feature 1..20: note = Synthetic
  source 1..20: mol_type = other RNA
tggagatggcagtttcctta

SEQ ID NO: 74 - RNA, 20 nt, synthetic construct
  misc_feature 1..20: note = Synthetic
  source 1..20: mol_type = other RNA
ggagatggcagtttccttag

SEQ ID NO: 75 - RNA, 20 nt, synthetic construct
  misc_feature 1..20: note = Synthetic
  source 1..20: mol_type = other RNA
gagatggcagtttccttagt

SEQ ID NO: 76 - RNA, 20 nt, synthetic construct
  misc_feature 1..20: note = Synthetic
  source 1..20: mol_type = other RNA
agatggcagtttccttagta

SEQ ID NO: 77 - RNA, 20 nt, synthetic construct
  misc_feature 1..20: note = Synthetic
  source 1..20: mol_type = other RNA
gatggcagtttccttagtaa

SEQ ID NO: 78 - RNA, 20 nt, synthetic construct
  misc_feature 1..20: note = Synthetic
  source 1..20: mol_type = other RNA
atggcagtttccttagtaac

SEQ ID NO: 79 - RNA, 20 nt, synthetic construct
  misc_feature 1..20: note = Synthetic
  source 1..20: mol_type = other RNA
tggcagtttccttagtaacc

SEQ ID NO: 80 - RNA, 20 nt, synthetic construct
  misc_feature 1..20: note = Synthetic
  source 1..20: mol_type = other RNA
ggcagtttccttagtaacca

SEQ ID NO: 81 - RNA, 20 nt, synthetic construct
  misc_feature 1..20: note = Synthetic
  source 1..20: mol_type = other RNA
gcagtttccttagtaaccac

SEQ ID NO: 82 - RNA, 20 nt, synthetic construct
  misc_feature 1..20: note = Synthetic
  source 1..20: mol_type = other RNA
cagtttccttagtaaccaca

SEQ ID NO: 83 - RNA, 20 nt, synthetic construct
  misc_feature 1..20: note = Synthetic
  source 1..20: mol_type = other RNA
agtttccttagtaaccacag

SEQ ID NO: 84 - RNA, 20 nt, synthetic construct
  misc_feature 1..20: note = Synthetic
  source 1..20: mol_type = other RNA
gtttccttagtaaccacagg

SEQ ID NO: 85 - RNA, 20 nt, synthetic construct
  misc_feature 1..20: note = Synthetic
  source 1..20: mol_type = other RNA
tttccttagtaaccacaggt

SEQ ID NO: 86 - RNA, 20 nt, synthetic construct
  misc_feature 1..20: note = Synthetic
  source 1..20: mol_type = other RNA
ttccttagtaaccacaggtt

SEQ ID NO: 87 - RNA, 20 nt, synthetic construct
  misc_feature 1..20: note = Synthetic
  source 1..20: mol_type = other RNA
tccttagtaaccacaggttg

SEQ ID NO: 88 - RNA, 20 nt, synthetic construct
  misc_feature 1..20: note = Synthetic
  source 1..20: mol_type = other RNA
ccttagtaaccacaggttgt

SEQ ID NO: 89 - RNA, 20 nt, synthetic construct
  misc_feature 1..20: note = Synthetic
  source 1..20: mol_type = other RNA
cttagtaaccacaggttgtg

SEQ ID NO: 90 - RNA, 20 nt, synthetic construct
  misc_feature 1..20: note = Synthetic
  source 1..20: mol_type = other RNA
ttagtaaccacaggttgtgt

SEQ ID NO: 91 - RNA, 20 nt, synthetic construct
  misc_feature 1..20: note = Synthetic
  source 1..20: mol_type = other RNA
tagtaaccacaggttgtgtc

SEQ ID NO: 92 - RNA, 20 nt, synthetic construct
  misc_feature 1..20: note = Synthetic
  source 1..20: mol_type = other RNA
agtaaccacaggttgtgtca

SEQ ID NO: 93 - RNA, 20 nt, synthetic construct
  misc_feature 1..20: note = Synthetic
  source 1..20: mol_type = other RNA
gtaaccacaggttgtgtcac

SEQ ID NO: 94 - RNA, 20 nt, synthetic construct
  misc_feature 1..20: note = Synthetic
  source 1..20: mol_type = other RNA
taaccacaggttgtgtcacc

SEQ ID NO: 95 - RNA, 20 nt, synthetic construct
  misc_feature 1..20: note = Synthetic
  source 1..20: mol_type = other RNA
aaccacaggttgtgtcacca

SEQ ID NO: 96 - RNA, 20 nt, synthetic construct
  misc_feature 1..20: note = Synthetic
  source 1..20: mol_type = other RNA
accacaggttgtgtcaccag

SEQ ID NO: 97 - RNA, 20 nt, synthetic construct
  misc_feature 1..20: note = Synthetic
  source 1..20: mol_type = other RNA
ccacaggttgtgtcaccaga

SEQ ID NO: 98 - RNA, 20 nt, synthetic construct
  misc_feature 1..20: note = Synthetic
  source 1..20: mol_type = other RNA
cacaggttgtgtcaccagag

SEQ ID NO: 99 - RNA, 20 nt, synthetic construct
  misc_feature 1..20: note = Synthetic
  source 1..20: mol_type = other RNA
acaggttgtgtcaccagagt

SEQ ID NO: 100 - RNA, 20 nt, synthetic construct
  misc_feature 1..20: note = Synthetic
  source 1..20: mol_type = other RNA
caggttgtgtcaccagagta

SEQ ID NO: 101 - RNA, 20 nt, synthetic construct
  misc_feature 1..20: note = Synthetic
  source 1..20: mol_type = other RNA
aggttgtgtcaccagagtaa

SEQ ID NO: 102 - RNA, 20 nt, synthetic construct
  misc_feature 1..20: note = Synthetic
  source 1..20: mol_type = other RNA
ggttgtgtcaccagagtaac

SEQ ID NO: 103 - RNA, 20 nt, synthetic construct
  misc_feature 1..20: note = Synthetic
  source 1..20: mol_type = other RNA
gttgtgtcaccagagtaaca

SEQ ID NO: 104 - RNA, 20 nt, synthetic construct
  misc_feature 1..20: note = Synthetic
  source 1..20: mol_type = other RNA
ttgtgtcaccagagtaacag

SEQ ID NO: 105 - RNA, 20 nt, synthetic construct
  misc_feature 1..20: note = Synthetic
  source 1..20: mol_type = other RNA
tgtgtcaccagagtaacagt

SEQ ID NO: 106 - RNA, 20 nt, synthetic construct
  misc_feature 1..20: note = Synthetic
  source 1..20: mol_type = other RNA
gtgtcaccagagtaacagtc

SEQ ID NO: 107 - RNA, 20 nt, synthetic construct
  misc_feature 1..20: note = Synthetic
  source 1..20: mol_type = other RNA
tgtcaccagagtaacagtct

SEQ ID NO: 108 - RNA, 20 nt, synthetic construct
  misc_feature 1..20: note = Synthetic
  source 1..20: mol_type = other RNA
gtcaccagagtaacagtctg

SEQ ID NO: 109 - RNA, 20 nt, synthetic construct
  misc_feature 1..20: note = Synthetic
  source 1..20: mol_type = other RNA
tcaccagagtaacagtctga

SEQ ID NO: 110 - RNA, 20 nt, synthetic construct
  misc_feature 1..20: note = Synthetic
  source 1..20: mol_type = other RNA
caccagagtaacagtctgag

SEQ ID NO: 111 - RNA, 20 nt, synthetic construct
  misc_feature 1..20: note = Synthetic
  source 1..20: mol_type = other RNA
accagagtaacagtctgagt

SEQ ID NO: 112 - RNA, 20 nt, synthetic construct
  misc_feature 1..20: note = Synthetic
  source 1..20: mol_type = other RNA
ccagagtaacagtctgagta

SEQ ID NO: 113 - RNA, 20 nt, synthetic construct
  misc_feature 1..20: note = Synthetic
  source 1..20: mol_type = other RNA
cagagtaacagtctgagtag

SEQ ID NO: 114 - RNA, 20 nt, synthetic construct
  misc_feature 1..20: note = Synthetic
  source 1..20: mol_type = other RNA
agagtaacagtctgagtagg

SEQ ID NO: 115 - RNA, 20 nt, synthetic construct
  misc_feature 1..20: note = Synthetic
  source 1..20: mol_type = other RNA
gagtaacagtctgagtagga

SEQ ID NO: 116 - RNA, 20 nt, synthetic construct
  misc_feature 1..20: note = Synthetic
  source 1..20: mol_type = other RNA
agtaacagtctgagtaggag

SEQ ID NO: 117 - RNA, 20 nt, synthetic construct
  misc_feature 1..20: note = Synthetic
  source 1..20: mol_type = other RNA
gtaacagtctgagtaggagc

SEQ ID NO: 118 - RNA, 20 nt, synthetic construct
  misc_feature 1..20: note = Synthetic
  source 1..20: mol_type = other RNA
taacagtctgagtaggagct

SEQ ID NO: 119 - RNA, 20 nt, synthetic construct
  misc_feature 1..20: note = Synthetic
  source 1..20: mol_type = other RNA
aacagtctgagtaggagcta

SEQ ID NO: 120 - RNA, 20 nt, synthetic construct
  misc_feature 1..20: note = Synthetic
  source 1..20: mol_type = other RNA
acagtctgagtaggagctaa

SEQ ID NO: 121 - RNA, 20 nt, synthetic construct
  misc_feature 1..20: note = Synthetic
  source 1..20: mol_type = other RNA
cagtctgagtaggagctaaa

SEQ ID NO: 122 - RNA, 20 nt, synthetic construct
  misc_feature 1..20: note = Synthetic
  source 1..20: mol_type = other RNA
agtctgagtaggagctaaaa

SEQ ID NO: 123 - RNA, 20 nt, synthetic construct
  misc_feature 1..20: note = Synthetic
  source 1..20: mol_type = other RNA
gtctgagtaggagctaaaat

SEQ ID NO: 124 - RNA, 20 nt, synthetic construct
  misc_feature 1..20: note = Synthetic
  source 1..20: mol_type = other RNA
tctgagtaggagctaaaata

SEQ ID NO: 125 - RNA, 20 nt, synthetic construct
  misc_feature 1..20: note = Synthetic
  source 1..20: mol_type = other RNA
ctgagtaggagctaaaatat

SEQ ID NO: 126 - RNA, 20 nt, synthetic construct
  misc_feature 1..20: note = Synthetic
  source 1..20: mol_type = other RNA
ggtaagttctgtccaagccc

SEQ ID NO: 127 - RNA, 20 nt, synthetic construct
  misc_feature 1..20: note = Synthetic
  source 1..20: mol_type = other RNA
gtacctccaacatcaaggaa

SEQ ID NO: 128 - RNA, 20 nt, synthetic construct
  misc_feature 1..20: note = Synthetic
  source 1..20: mol_type = other RNA
caacatcaaggaagatggca

SEQ ID NO: 129 - RNA, 20 nt, synthetic construct
  misc_feature 1..20: note = Synthetic
  source 1..20: mol_type = other RNA
gatggcatttctagtttgga

SEQ ID NO: 130 - RNA, 20 nt, synthetic construct
  misc_feature 1..20: note = Synthetic
  source 1..20: mol_type = other RNA
atggcatttctagtttggag

SEQ ID NO: 131 - RNA, 20 nt, synthetic construct
  misc_feature 1..20: note = Synthetic
  source 1..20: mol_type = other RNA
tggcatttctagtttggaga

SEQ ID NO: 132 - RNA, 20 nt, synthetic construct
  misc_feature 1..20: note = Synthetic
  source 1..20: mol_type = other RNA
ggcatttctagtttggagat

SEQ ID NO: 133 - RNA, 20 nt, synthetic construct
  misc_feature 1..20: note = Synthetic
  source 1..20: mol_type = other RNA
gcatttctagtttggagatg

SEQ ID NO: 134 - RNA, 20 nt, synthetic construct
  misc_feature 1..20: note = Synthetic
  source 1..20: mol_type = other RNA
gcagtttccttagtaaccac

SEQ ID NO: 135 - RNA, 20 nt, synthetic construct
  misc_feature 1..20: note = Synthetic
  source 1..20: mol_type = other RNA
cagtttccttagtaaccaca

SEQ ID NO: 136 - RNA, 20 nt, synthetic construct
  misc_feature 1..20: note = Synthetic
  source 1..20: mol_type = other RNA
ttccttagtaaccacaggtt

SEQ ID NO: 137 - RNA, 20 nt, synthetic construct
  misc_feature 1..20: note = Synthetic
  source 1..20: mol_type = other RNA
ttgtgtcaccagagtaacag

SEQ ID NO: 138 - RNA, 19 nt, synthetic construct
  misc_feature 1..19: note = Synthetic
  source 1..19: mol_type = other RNA
cagtttccttagtaaccac

SEQ ID NO: 139 - RNA, 19 nt, synthetic construct
  misc_feature 1..19: note = Synthetic
  source 1..19: mol_type = other RNA
gtttccttagtaaccacag

SEQ ID NO: 140 - RNA, 17 nt, synthetic construct
  misc_feature 1..17: note = Synthetic
  source 1..17: mol_type = other RNA
gtaagttctgtccaagc

SEQ ID NO: 141 - RNA, 17 nt, synthetic construct
  misc_feature 1..17: note = Synthetic
  source 1..17: mol_type = other RNA
gtaagttctgtccaagc

SEQ ID NO: 142 - RNA, 19 nt, synthetic construct
  misc_feature 1..19: note = Synthetic
  source 1..19: mol_type = other RNA
tacctccaacatcaaggaa

SEQ ID NO: 143 - RNA, 20 nt, synthetic construct
  misc_feature 1..20: note = Synthetic
  source 1..20: mol_type = other RNA
gtacctccaacatcaaggaa

SEQ ID NO: 144 - RNA, 20 nt, synthetic construct
  misc_feature 1..20: note = Synthetic
  source 1..20: mol_type = other RNA
tggcatttctagtttggaga

SEQ ID NO: 145 - RNA, 20 nt, synthetic construct
  misc_feature 1..20: note = Synthetic
  source 1..20: mol_type = other RNA
ggcatttctagtttggagat

SEQ ID NO: 146 - RNA, 20 nt, synthetic construct
  misc_feature 1..20: note = Synthetic
  source 1..20: mol_type = other RNA
tggcagtttccttagtaacc

SEQ ID NO: 147 - RNA, 20 nt, synthetic construct
  misc_feature 1..20: note = Synthetic
  source 1..20: mol_type = other RNA
ggtaagttctgtccaagccc

SEQ ID NO: 148 - RNA, 20 nt, synthetic construct
  misc_feature 1..20: note = Synthetic
  source 1..20: mol_type = other RNA
gtacctccaacatcaaggaa

SEQ ID NO: 149 - RNA, 20 nt, synthetic construct
  misc_feature 1..20: note = Synthetic
  source 1..20: mol_type = other RNA
caacatcaaggaagatggca

SEQ ID NO: 150 - RNA, 20 nt, synthetic construct
  misc_feature 1..20: note = Synthetic
  source 1..20: mol_type = other RNA
atggcatttctagtttggag

SEQ ID NO: 151 - RNA, 20 nt, synthetic construct
  misc_feature 1..20: note = Synthetic
  source 1..20: mol_type = other RNA
tggcatttctagtttggaga

SEQ ID NO: 152 - RNA, 20 nt, synthetic construct
  misc_feature 1..20: note = Synthetic
  source 1..20: mol_type = other RNA
ggcatttctagtttggagat

SEQ ID NO: 153 - RNA, 20 nt, synthetic construct
  misc_feature 1..20: note = Synthetic
  source 1..20: mol_type = other RNA
gcatttctagtttggagatg

SEQ ID NO: 154 - RNA, 20 nt, synthetic construct
  misc_feature 1..20: note = Synthetic
  source 1..20: mol_type = other RNA
tggcagtttccttagtaacc

SEQ ID NO: 155 - RNA, 20 nt, synthetic construct
  misc_feature 1..20: note = Synthetic
  source 1..20: mol_type = other RNA
cagtttccttagtaaccaca

SEQ ID NO: 156 - RNA, 20 nt, synthetic construct
  misc_feature 1..20: note = Synthetic
  source 1..20: mol_type = other RNA
agtttccttagtaaccacag

SEQ ID NO: 157 - RNA, 20 nt, synthetic construct
  misc_feature 1..20: note = Synthetic
  source 1..20: mol_type = other RNA
ttccttagtaaccacaggtt

SEQ ID NO: 158 - RNA, 20 nt, synthetic construct
  misc_feature 1..20: note = Synthetic
  source 1..20: mol_type = other RNA
ttgtgtcaccagagtaacag

SEQ ID NO: 159 - RNA, 20 nt, synthetic construct
  misc_feature 1..20: note = Synthetic
  source 1..20: mol_type = other RNA
ggtaagttctgtccaagccc

SEQ ID NO: 160 - RNA, 20 nt, synthetic construct
  misc_feature 1..20: note = Synthetic
  source 1..20: mol_type = other RNA
gtacctccaacatcaaggaa

SEQ ID NO: 161 - RNA, 20 nt, synthetic construct
  misc_feature 1..20: note = Synthetic
  source 1..20: mol_type = other RNA
caacatcaaggaagatggca

SEQ ID NO: 162 - RNA, 20 nt, synthetic construct
  misc_feature 1..20: note = Synthetic
  source 1..20: mol_type = other RNA
atggcatttctagtttggag

SEQ ID NO: 163 - RNA, 20 nt, synthetic construct
  misc_feature 1..20: note = Synthetic
  source 1..20: mol_type = other RNA
tggcatttctagtttggaga

SEQ ID NO: 164 - RNA, 20 nt, synthetic construct
  misc_feature 1..20: note = Synthetic
  source 1..20: mol_type = other RNA
ggcatttctagtttggagat

SEQ ID NO: 165 - RNA, 20 nt, synthetic construct
  misc_feature 1..20: note = Synthetic
  source 1..20: mol_type = other RNA
gcatttctagtttggagatg

SEQ ID NO: 166 - RNA, 20 nt, synthetic construct
  misc_feature 1..20: note = Synthetic
  source 1..20: mol_type = other RNA
tggcagtttccttagtaacc

SEQ ID NO: 167 - RNA, 20 nt, synthetic construct
  misc_feature 1..20: note = Synthetic
  source 1..20: mol_type = other RNA
cagtttccttagtaaccaca

SEQ ID NO: 168 - RNA, 20 nt, synthetic construct
  misc_feature 1..20: note = Synthetic
  source 1..20: mol_type = other RNA
agtttccttagtaaccacag

SEQ ID NO: 169 - RNA, 20 nt, synthetic construct
  misc_feature 1..20: note = Synthetic
  source 1..20: mol_type = other RNA
ttccttagtaaccacaggtt

SEQ ID NO: 170 - RNA, 20 nt, synthetic construct
  misc_feature 1..20: note = Synthetic
  source 1..20: mol_type = other RNA
ttgtgtcaccagagtaacag

SEQ ID NO: 171 - RNA, 20 nt, synthetic construct
  misc_feature 1..20: note = Synthetic
  source 1..20: mol_type = other RNA
ggtaagttctgtccaagccc

SEQ ID NO: 172 - RNA, 20 nt, synthetic construct
  misc_feature 1..20: note = Synthetic
  source 1..20: mol_type = other RNA
gtacctccaacatcaaggaa

SEQ ID NO: 173 - RNA, 20 nt, synthetic construct
  misc_feature 1..20: note = Synthetic
  source 1..20: mol_type = other RNA
caacatcaaggaagatggca

SEQ ID NO: 174 - RNA, 20 nt, synthetic construct
  misc_feature 1..20: note = Synthetic
  source 1..20: mol_type = other RNA
atggcatttctagtttggag

SEQ ID NO: 175 - RNA, 20 nt, synthetic construct
  misc_feature 1..20: note = Synthetic
  source 1..20: mol_type = other RNA
tggcatttctagtttggaga

SEQ ID NO: 176 - RNA, 20 nt, synthetic construct
  misc_feature 1..20: note = Synthetic
  source 1..20: mol_type = other RNA
ggcatttctagtttggagat

SEQ ID NO: 177 - RNA, 20 nt, synthetic construct
  misc_feature 1..20: note = Synthetic
  source 1..20: mol_type = other RNA
gcatttctagtttggagatg

SEQ ID NO: 178 - RNA, 20 nt, synthetic construct
  misc_feature 1..20: note = Synthetic
  source 1..20: mol_type = other RNA
tggcagtttccttagtaacc

SEQ ID NO: 179 - RNA, 20 nt, synthetic construct
  misc_feature 1..20: note = Synthetic
  source 1..20: mol_type = other RNA
cagtttccttagtaaccaca

SEQ ID NO: 180 - RNA, 20 nt, synthetic construct
  misc_feature 1..20: note = Synthetic
  source 1..20: mol_type = other RNA
agtttccttagtaaccacag

SEQ ID NO: 181 - RNA, 20 nt, synthetic construct
  misc_feature 1..20: note = Synthetic
  source 1..20: mol_type = other RNA
ttccttagtaaccacaggtt

SEQ ID NO: 182 - RNA, 20 nt, synthetic construct
  misc_feature 1..20: note = Synthetic
  source 1..20: mol_type = other RNA
ttgtgtcaccagagtaacag

SEQ ID NO: 183 - RNA, 20 nt, synthetic construct
  misc_feature 1..20: note = Synthetic
  source 1..20: mol_type = other RNA
ggtaagttctgtccaagccc

SEQ ID NO: 184 - RNA, 20 nt, synthetic construct
  misc_feature 1..20: note = Synthetic
  source 1..20: mol_type = other RNA
gtacctccaacatcaaggaa

SEQ ID NO: 185 - RNA, 20 nt, synthetic construct
  misc_feature 1..20: note = Synthetic
  source 1..20: mol_type = other RNA
caacatcaaggaagatggca

SEQ ID NO: 186 - RNA, 20 nt, synthetic construct
  misc_feature 1..20: note = Synthetic
  source 1..20: mol_type = other RNA
atggcatttctagtttggag

SEQ ID NO: 187 - RNA, 20 nt, synthetic construct
  misc_feature 1..20: note = Synthetic
  source 1..20: mol_type = other RNA
tggcatttctagtttggaga

SEQ ID NO: 188 - RNA, 20 nt, synthetic construct
  misc_feature 1..20: note = Synthetic
  source 1..20: mol_type = other RNA
ggcatttctagtttggagat

SEQ ID NO: 189 - RNA, 20 nt, synthetic construct
  misc_feature 1..20: note = Synthetic
  source 1..20: mol_type = other RNA
gcatttctagtttggagatg

SEQ ID NO: 190 - RNA, 20 nt, synthetic construct
  misc_feature 1..20: note = Synthetic
  source 1..20: mol_type = other RNA
tggcagtttccttagtaacc

SEQ ID NO: 191 - RNA, 20 nt, synthetic construct
  misc_feature 1..20: note = Synthetic
  source 1..20: mol_type = other RNA
cagtttccttagtaaccaca

SEQ ID NO: 192 - RNA, 20 nt, synthetic construct
  misc_feature 1..20: note = Synthetic
  source 1..20: mol_type = other RNA
agtttccttagtaaccacag

SEQ ID NO: 193 - RNA, 20 nt, synthetic construct
  misc_feature 1..20: note = Synthetic
  source 1..20: mol_type = other RNA
ttccttagtaaccacaggtt

SEQ ID NO: 194 - RNA, 20 nt, synthetic construct
  misc_feature 1..20: note = Synthetic
  source 1..20: mol_type = other RNA
ttgtgtcaccagagtaacag

SEQ ID NO: 195 - RNA, 20 nt, synthetic construct
  misc_feature 1..20: note = Synthetic
  source 1..20: mol_type = other RNA
tcaaggaagatggcatttct

SEQ ID NO: 196 - RNA, 20 nt, synthetic construct
  misc_feature 1..20: note = Synthetic
  source 1..20: mol_type = other RNA
tcaaggaagatggcatttct

SEQ ID NO: 197 - RNA, 20 nt, synthetic construct
  misc_feature 1..20: note = Synthetic
  source 1..20: mol_type = other RNA
tcaaggaagatggcatttct

SEQ ID NO: 198 - RNA, 20 nt, synthetic construct
  misc_feature 1..20: note = Synthetic
  source 1..20: mol_type = other RNA
tcaaggaagatggcatttct

SEQ ID NO: 199 - RNA, 20 nt, synthetic construct
  misc_feature 1..20: note = Synthetic
  source 1..20: mol_type = other RNA
cttctgccaacttttatcat

SEQ ID NO: 200 - RNA, 20 nt, synthetic construct
  misc_feature 1..20: note = Synthetic
  source 1..20: mol_type = other RNA
ttctgccaacttttatcatt

SEQ ID NO: 201 - RNA, 20 nt, synthetic construct
  misc_feature 1..20: note = Synthetic
  source 1..20: mol_type = other RNA
tctgccaacttttatcattt

SEQ ID NO: 202 - RNA, 20 nt, synthetic construct
  misc_feature 1..20: note = Synthetic
  source 1..20: mol_type = other RNA
ctgccaacttttatcatttt

SEQ ID NO: 203 - RNA, 20 nt, synthetic construct
  misc_feature 1..20: note = Synthetic
  source 1..20: mol_type = other RNA
tgccaacttttatcattttt

SEQ ID NO: 204 - RNA, 20 nt, synthetic construct
  misc_feature 1..20: note = Synthetic
  source 1..20: mol_type = other RNA
gccaacttttatcatttttt

SEQ ID NO: 205 - RNA, 20 nt, synthetic construct
  misc_feature 1..20: note = Synthetic
  source 1..20: mol_type = other RNA
ccaacttttatcattttttc

SEQ ID NO: 206 - RNA, 20 nt, synthetic construct
  misc_feature 1..20: note = Synthetic
  source 1..20: mol_type = other RNA
caacttttatcattttttct

SEQ ID NO: 207 - RNA, 20 nt, synthetic construct
  misc_feature 1..20: note = Synthetic
  source 1..20: mol_type = other RNA
aacttttatcattttttctc

SEQ ID NO: 208 - RNA, 20 nt, synthetic construct
  misc_feature 1..20: note = Synthetic
  source 1..20: mol_type = other RNA
acttttatcattttttctca

SEQ ID NO: 209 - RNA, 20 nt, synthetic construct
  misc_feature 1..20: note = Synthetic
  source 1..20: mol_type = other RNA
cttttatcattttttctcat

SEQ ID NO: 210 - RNA, 20 nt, synthetic construct
  misc_feature 1..20: note = Synthetic
  source 1..20: mol_type = other RNA
ttttatcattttttctcata

SEQ ID NO: 211 - RNA, 20 nt, synthetic construct
  misc_feature 1..20: note = Synthetic
  source 1..20: mol_type = other RNA
tttatcattttttctcatac

SEQ ID NO: 212 - RNA, 20 nt, synthetic construct
  misc_feature 1..20: note = Synthetic
  source 1..20: mol_type = other RNA
ttatcattttttctcatacc

SEQ ID NO: 213 - RNA, 20 nt, synthetic construct
  misc_feature 1..20: note = Synthetic
  source 1..20: mol_type = other RNA
tatcattttttctcatacct

SEQ ID NO: 214 - RNA, 20 nt, synthetic construct
  misc_feature 1..20: note = Synthetic
  source 1..20: mol_type = other RNA
atcattttttctcatacctt

SEQ ID NO: 215 - RNA, 20 nt, synthetic construct
  misc_feature 1..20: note = Synthetic
  source 1..20: mol_type = other RNA
tcattttttctcataccttc

SEQ ID NO: 216 - RNA, 20 nt, synthetic construct
  misc_feature 1..20: note = Synthetic
  source 1..20: mol_type = other RNA
cattttttctcataccttct

SEQ ID NO: 217 - RNA, 20 nt, synthetic construct
  misc_feature 1..20: note = Synthetic
  source 1..20: mol_type = other RNA
attttttctcataccttctg

SEQ ID NO: 218 - RNA, 20 nt, synthetic construct
  misc_feature 1..20: note = Synthetic
  source 1..20: mol_type = other RNA
ttttttctcataccttctgc

SEQ ID NO: 219 - RNA, 20 nt, synthetic construct
  misc_feature 1..20: note = Synthetic
  source 1..20: mol_type = other RNA
tttttctcataccttctgct

SEQ ID NO: 220 - RNA, 20 nt, synthetic construct
  misc_feature 1..20: note = Synthetic
  source 1..20: mol_type = other RNA
ttttctcataccttctgctt

SEQ ID NO: 221 - RNA, 20 nt, synthetic construct
  misc_feature 1..20: note = Synthetic
  source 1..20: mol_type = other RNA
tttctcataccttctgcttg

SEQ ID NO: 222 - RNA, 20 nt, synthetic construct
  misc_feature 1..20: note = Synthetic
  source 1..20: mol_type = other RNA
ttctcataccttctgcttga

SEQ ID NO: 223 - RNA, 20 nt, synthetic construct
  misc_feature 1..20: note = Synthetic
  source 1..20: mol_type = other RNA
tctcataccttctgcttgat

SEQ ID NO: 224 - RNA, 20 nt, synthetic construct
  misc_feature 1..20: note = Synthetic
  source 1..20: mol_type = other RNA
ctcataccttctgcttgatg

SEQ ID NO: 225 - RNA, 20 nt, synthetic construct
  misc_feature 1..20: note = Synthetic
  source 1..20: mol_type = other RNA
tcataccttctgcttgatga

SEQ ID NO: 226 - RNA, 20 nt, synthetic construct
  misc_feature 1..20: note = Synthetic
  source 1..20: mol_type = other RNA
cataccttctgcttgatgat

SEQ ID NO: 227 - RNA, 20 nt, synthetic construct
  misc_feature 1..20: note = Synthetic
  source 1..20: mol_type = other RNA
ataccttctgcttgatgatc

SEQ ID NO: 228 - RNA, 20 nt, synthetic construct
  misc_feature 1..20: note = Synthetic
  source 1..20: mol_type = other RNA
taccttctgcttgatgatca

SEQ ID NO: 229 - RNA, 20 nt, synthetic construct
  misc_feature 1..20: note = Synthetic
  source 1..20: mol_type = other RNA
accttctgcttgatgatcat

SEQ ID NO: 230 - RNA, 20 nt, synthetic construct
  misc_feature 1..20: note = Synthetic
  source 1..20: mol_type = other RNA
ccttctgcttgatgatcatc

SEQ ID NO: 231 - RNA, 20 nt, synthetic construct
  misc_feature 1..20: note = Synthetic
  source 1..20: mol_type = other RNA
cttctgcttgatgatcatct

SEQ ID NO: 232 - RNA, 20 nt, synthetic construct
  misc_feature 1..20: note = Synthetic
  source 1..20: mol_type = other RNA
ttctgcttgatgatcatctc

SEQ ID NO: 233 - RNA, 20 nt, synthetic construct
  misc_feature 1..20: note = Synthetic
  source 1..20: mol_type = other RNA
tctgcttgatgatcatctcg

SEQ ID NO: 234 - RNA, 20 nt, synthetic construct
  misc_feature 1..20: note = Synthetic
  source 1..20: mol_type = other RNA
ctgcttgatgatcatctcgt

SEQ ID NO: 235 - RNA, 20 nt, synthetic construct
  misc_feature 1..20: note = Synthetic
  source 1..20: mol_type = other RNA
tgcttgatgatcatctcgtt

SEQ ID NO: 236 - RNA, 20 nt, synthetic construct
  misc_feature 1..20: note = Synthetic
  source 1..20: mol_type = other RNA
gcttgatgatcatctcgttg

SEQ ID NO: 237 - RNA, 20 nt, synthetic construct
  misc_feature 1..20: note = Synthetic
  source 1..20: mol_type = other RNA
cttgatgatcatctcgttga

SEQ ID NO: 238 - RNA, 20 nt, synthetic construct
  misc_feature 1..20: note = Synthetic
  source 1..20: mol_type = other RNA
ttgatgatcatctcgttgat

SEQ ID NO: 239 - RNA, 20 nt, synthetic construct
  misc_feature 1..20: note = Synthetic
  source 1..20: mol_type = other RNA
tgatgatcatctcgttgata

SEQ ID NO: 240 - RNA, 20 nt, synthetic construct
  misc_feature 1..20: note = Synthetic
  source 1..20: mol_type = other RNA
gatgatcatctcgttgatat

SEQ ID NO: 241 - RNA, 20 nt, synthetic construct
  misc_feature 1..20: note = Synthetic
  source 1..20: mol_type = other RNA
atgatcatctcgttgatatc

SEQ ID NO: 242 - RNA, 20 nt, synthetic construct
  misc_feature 1..20: note = Synthetic
  source 1..20: mol_type = other RNA
tgatcatctcgttgatatcc

SEQ ID NO: 243 - RNA, 20 nt, synthetic construct
  misc_feature 1..20: note = Synthetic
  source 1..20: mol_type = other RNA
gatcatctcgttgatatcct

SEQ ID NO: 244 - RNA, 20 nt, synthetic construct
  misc_feature 1..20: note = Synthetic
  source 1..20: mol_type = other RNA
atcatctcgttgatatcctc

SEQ ID NO: 245 - RNA, 20 nt, synthetic construct
  misc_feature 1..20: note = Synthetic
  source 1..20: mol_type = other RNA
tcatctcgttgatatcctca

SEQ ID NO: 246 - RNA, 20 nt, synthetic construct
  misc_feature 1..20: note = Synthetic
  source 1..20: mol_type = other RNA
catctcgttgatatcctcaa

SEQ ID NO: 247 - RNA, 20 nt, synthetic construct
  misc_feature 1..20: note = Synthetic
  source 1..20: mol_type = other RNA
atctcgttgatatcctcaag

SEQ ID NO: 248 - RNA, 20 nt, synthetic construct
  misc_feature 1..20: note = Synthetic
  source 1..20: mol_type = other RNA
tctcgttgatatcctcaagg

SEQ ID NO: 249 - RNA, 20 nt, synthetic construct
  misc_feature 1..20: note = Synthetic
  source 1..20: mol_type = other RNA
ctcgttgatatcctcaaggt

SEQ ID NO: 250 - RNA, 20 nt, synthetic construct
  misc_feature 1..20: note = Synthetic
  source 1..20: mol_type = other RNA
tcgttgatatcctcaaggtc

SEQ ID NO: 251 - RNA, 20 nt, synthetic construct
  misc_feature 1..20: note = Synthetic
  source 1..20: mol_type = other RNA
cgttgatatcctcaaggtca

SEQ ID NO: 252 - RNA, 20 nt, synthetic construct
  misc_feature 1..20: note = Synthetic
  source 1..20: mol_type = other RNA
gttgatatcctcaaggtcac

SEQ ID NO: 253 - RNA, 20 nt, synthetic construct
  misc_feature 1..20: note = Synthetic
  source 1..20: mol_type = other RNA
ttgatatcctcaaggtcacc

SEQ ID NO: 254 - RNA, 20 nt, synthetic construct
  misc_feature 1..20: note = Synthetic
  source 1..20: mol_type = other RNA
tgatatcctcaaggtcaccc

SEQ ID NO: 255 - RNA, 20 nt, synthetic construct
  misc_feature 1..20: note = Synthetic
  source 1..20: mol_type = other RNA
gatatcctcaaggtcaccca

SEQ ID NO: 256 - RNA, 20 nt, synthetic construct
  misc_feature 1..20: note = Synthetic
  source 1..20: mol_type = other RNA
atatcctcaaggtcacccac

SEQ ID NO: 257 - RNA, 20 nt, synthetic construct
  misc_feature 1..20: note = Synthetic
  source 1..20: mol_type = other RNA
tatcctcaaggtcacccacc

SEQ ID NO: 258 - RNA, 20 nt, synthetic construct
  misc_feature 1..20: note = Synthetic
  source 1..20: mol_type = other RNA
atcctcaaggtcacccacca

SEQ ID NO: 259 - RNA, 20 nt, synthetic construct
  misc_feature 1..20: note = Synthetic
  source 1..20: mol_type = other RNA
tcctcaaggtcacccaccat

SEQ ID NO: 260 - RNA, 20 nt, synthetic construct
  misc_feature 1..20: note = Synthetic
  source 1..20: mol_type = other RNA
cctcaaggtcacccaccatc

SEQ ID NO: 261 - RNA, 20 nt, synthetic construct
  misc_feature 1..20: note = Synthetic
  source 1..20: mol_type = other RNA
ctcaaggtcacccaccatca

SEQ ID NO: 262 - RNA, 20 nt, synthetic construct
  misc_feature 1..20: note = Synthetic
  source 1..20: mol_type = other RNA
tcaaggtcacccaccatcac

SEQ ID NO: 263 - RNA, 20 nt, synthetic construct
  misc_feature 1..20: note = Synthetic
  source 1..20: mol_type = other RNA
caaggtcacccaccatcacc

SEQ ID NO: 264 - RNA, 20 nt, synthetic construct
  misc_feature 1..20: note = Synthetic
  source 1..20: mol_type = other RNA
aaggtcacccaccatcaccc

SEQ ID NO: 265 - RNA, 20 nt, synthetic construct
  misc_feature 1..20: note = Synthetic
  source 1..20: mol_type = other RNA
aggtcacccaccatcaccct

SEQ ID NO: 266 - RNA, 20 nt, synthetic construct
  misc_feature 1..20: note = Synthetic
  source 1..20: mol_type = other RNA
ggtcacccaccatcaccctc

SEQ ID NO: 267 - RNA, 20 nt, synthetic construct
  misc_feature 1..20: note = Synthetic
  source 1..20: mol_type = other RNA
acccaccatcaccctctgtg

SEQ ID NO: 268 - RNA, 20 nt, synthetic construct
  misc_feature 1..20: note = Synthetic
  source 1..20: mol_type = other RNA
cccaccatcaccctctgtga

SEQ ID NO: 269 - RNA, 20 nt, synthetic construct
  misc_feature 1..20: note = Synthetic
  source 1..20: mol_type = other RNA
ccaccatcaccctctgtgat

SEQ ID NO: 270 - RNA, 20 nt, synthetic construct
  misc_feature 1..20: note = Synthetic
  source 1..20: mol_type = other RNA
caccatcaccctctgtgatt

SEQ ID NO: 271 - RNA, 20 nt, synthetic construct
  misc_feature 1..20: note = Synthetic
  source 1..20: mol_type = other RNA
accatcaccctctgtgattt

SEQ ID NO: 272 - RNA, 20 nt, synthetic construct
  misc_feature 1..20: note = Synthetic
  source 1..20: mol_type = other RNA
ccatcaccctctgtgatttt

SEQ ID NO: 273 - RNA, 20 nt, synthetic construct
  misc_feature 1..20: note = Synthetic
  source 1..20: mol_type = other RNA
catcaccctctgtgatttta

SEQ ID NO: 274 - RNA, 20 nt, synthetic construct
  misc_feature 1..20: note = Synthetic
  source 1..20: mol_type = other RNA
atcaccctctgtgattttat

SEQ ID NO: 275 - RNA, 20 nt, synthetic construct
  misc_feature 1..20: note = Synthetic
  source 1..20: mol_type = other RNA
tcaccctctgtgattttata

SEQ ID NO: 276 - RNA, 20 nt, synthetic construct
  misc_feature 1..20: note = Synthetic
  source 1..20: mol_type = other RNA
caccctctgtgattttataa

SEQ ID NO: 277 - RNA, 20 nt, synthetic construct
  misc_feature 1..20: note = Synthetic
  source 1..20: mol_type = other RNA
accctctgtgattttataac

SEQ ID NO: 278 - RNA, 20 nt, synthetic construct
  misc_feature 1..20: note = Synthetic
  source 1..20: mol_type = other RNA
ccctctgtgattttataact

SEQ ID NO: 279 - RNA, 20 nt, synthetic construct
  misc_feature 1..20: note = Synthetic
  source 1..20: mol_type = other RNA
cctctgtgattttataactt

SEQ ID NO: 280 - RNA, 20 nt, synthetic construct
  misc_feature 1..20: note = Synthetic
  source 1..20: mol_type = other RNA
ctctgtgattttataacttg

SEQ ID NO: 281 - RNA, 20 nt, synthetic construct
  misc_feature 1..20: note = Synthetic
  source 1..20: mol_type = other RNA
tctgtgattttataacttga

SEQ ID NO: 282 - RNA, 20 nt, synthetic construct
  misc_feature 1..20: note = Synthetic
  source 1..20: mol_type = other RNA
ctgtgattttataacttgat

SEQ ID NO: 283 - RNA, 20 nt, synthetic construct
  misc_feature 1..20: note = Synthetic
  source 1..20: mol_type = other RNA
tgtgattttataacttgatc

SEQ ID NO: 284 - RNA, 20 nt, synthetic construct
  misc_feature 1..20: note = Synthetic
  source 1..20: mol_type = other RNA
gtgattttataacttgatca

SEQ ID NO: 285 - RNA, 20 nt, synthetic construct
  misc_feature 1..20: note = Synthetic
  source 1..20: mol_type = other RNA
tgattttataacttgatcaa

SEQ ID NO: 286 - RNA, 20 nt, synthetic construct
  misc_feature 1..20: note = Synthetic
  source 1..20: mol_type = other RNA
gattttataacttgatcaag

SEQ ID NO: 287 - RNA, 20 nt, synthetic construct
  misc_feature 1..20: note = Synthetic
  source 1..20: mol_type = other RNA
attttataacttgatcaagc

SEQ ID NO: 288 - RNA, 20 nt, synthetic construct
  misc_feature 1..20: note = Synthetic
  source 1..20: mol_type = other RNA
ttttataacttgatcaagca

SEQ ID NO: 289 - RNA, 20 nt, synthetic construct
  misc_feature 1..20: note = Synthetic
  source 1..20: mol_type = other RNA
tttataacttgatcaagcag

SEQ ID NO: 290 - RNA, 20 nt, synthetic construct
  misc_feature 1..20: note = Synthetic
  source 1..20: mol_type = other RNA
ttataacttgatcaagcaga

SEQ ID NO: 291 - RNA, 20 nt, synthetic construct
  misc_feature 1..20: note = Synthetic
  source 1..20: mol_type = other RNA
tataacttgatcaagcagag

SEQ ID NO: 292 - RNA, 20 nt, synthetic construct
  misc_feature 1..20: note = Synthetic
  source 1..20: mol_type = other RNA
ataacttgatcaagcagaga

SEQ ID NO: 293 - RNA, 20 nt, synthetic construct
  misc_feature 1..20: note = Synthetic
  source 1..20: mol_type = other RNA
taacttgatcaagcagagaa

SEQ ID NO: 294 - RNA, 20 nt, synthetic construct
  misc_feature 1..20: note = Synthetic
  source 1..20: mol_type = other RNA
aacttgatcaagcagagaaa

SEQ ID NO: 295 - RNA, 20 nt, synthetic construct
  misc_feature 1..20: note = Synthetic
  source 1..20: mol_type = other RNA
acttgatcaagcagagaaag

SEQ ID NO: 296 - RNA, 20 nt, synthetic construct
  misc_feature 1..20: note = Synthetic
  source 1..20: mol_type = other RNA
cttgatcaagcagagaaagc

SEQ ID NO: 297 - RNA, 20 nt, synthetic construct
  misc_feature 1..20: note = Synthetic
  source 1..20: mol_type = other RNA
ttgatcaagcagagaaagcc

SEQ ID NO: 298 - RNA, 20 nt, synthetic construct
  misc_feature 1..20: note = Synthetic
  source 1..20: mol_type = other RNA
tgatcaagcagagaaagcca

SEQ ID NO: 299 - RNA, 20 nt, synthetic construct
  misc_feature 1..20: note = Synthetic
  source 1..20: mol_type = other RNA
gatcaagcagagaaagccag

SEQ ID NO: 300 - RNA, 20 nt, synthetic construct
  misc_feature 1..20: note = Synthetic
  source 1..20: mol_type = other RNA
gatcaagcagagaaagccag

SEQ ID NO: 301 - RNA, 20 nt, synthetic construct
  misc_feature 1..20: note = Synthetic
  source 1..20: mol_type = other RNA
atcaagcagagaaagccagt

SEQ ID NO: 302 - RNA, 20 nt, synthetic construct
  misc_feature 1..20: note = Synthetic
  source 1..20: mol_type = other RNA
tcaagcagagaaagccagtc

SEQ ID NO: 303 - RNA, 20 nt, synthetic construct
  misc_feature 1..20: note = Synthetic
  source 1..20: mol_type = other RNA
caagcagagaaagccagtcg

SEQ ID NO: 304 - RNA, 20 nt, synthetic construct
  misc_feature 1..20: note = Synthetic
  source 1..20: mol_type = other RNA
aagcagagaaagccagtcgg

SEQ ID NO: 305 - RNA, 20 nt, synthetic construct
  misc_feature 1..20: note = Synthetic
  source 1..20: mol_type = other RNA
agcagagaaagccagtcggt

SEQ ID NO: 306 - RNA, 20 nt, synthetic construct
  misc_feature 1..20: note = Synthetic
  source 1..20: mol_type = other RNA
gcagagaaagccagtcggta

SEQ ID NO: 307 - RNA, 20 nt, synthetic construct
  misc_feature 1..20: note = Synthetic
  source 1..20: mol_type = other RNA
cagagaaagccagtcggtaa

SEQ ID NO: 308 - RNA, 20 nt, synthetic construct
  misc_feature 1..20: note = Synthetic
  source 1..20: mol_type = other RNA
agagaaagccagtcggtaag

SEQ ID NO: 309 - RNA, 20 nt, synthetic construct
  misc_feature 1..20: note = Synthetic
  source 1..20: mol_type = other RNA
gagaaagccagtcggtaagt

SEQ ID NO: 310 - RNA, 20 nt, synthetic construct
  misc_feature 1..20: note = Synthetic
  source 1..20: mol_type = other RNA
agaaagccagtcggtaagtt

SEQ ID NO: 311 - RNA, 20 nt, synthetic construct
  misc_feature 1..20: note = Synthetic
  source 1..20: mol_type = other RNA
gaaagccagtcggtaagttc

SEQ ID NO: 312 - RNA, 20 nt, synthetic construct
  misc_feature 1..20: note = Synthetic
  source 1..20: mol_type = other RNA
aaagccagtcggtaagttct

SEQ ID NO: 313 - RNA, 20 nt, synthetic construct
  misc_feature 1..20: note = Synthetic
  source 1..20: mol_type = other RNA
aagccagtcggtaagttctg

SEQ ID NO: 314 - RNA, 20 nt, synthetic construct
  misc_feature 1..20: note = Synthetic
  source 1..20: mol_type = other RNA
agccagtcggtaagttctgt

SEQ ID NO: 315 - RNA, 20 nt, synthetic construct
  misc_feature 1..20: note = Synthetic
  source 1..20: mol_type = other RNA
gccagtcggtaagttctgtc

SEQ ID NO: 316 - RNA, 20 nt, synthetic construct
  misc_feature 1..20: note = Synthetic
  source 1..20: mol_type = other RNA
ccagtcggtaagttctgtcc

SEQ ID NO: 317 - RNA, 20 nt, synthetic construct
  misc_feature 1..20: note = Synthetic
  source 1..20: mol_type = other RNA
cagtcggtaagttctgtcca

SEQ ID NO: 318 - RNA, 20 nt, synthetic construct
  misc_feature 1..20: note = Synthetic
  source 1..20: mol_type = other RNA
agtcggtaagttctgtccaa

SEQ ID NO: 319 - RNA, 20 nt, synthetic construct
  misc_feature 1..20: note = Synthetic
  source 1..20: mol_type = other RNA
gtcggtaagttctgtccaag

SEQ ID NO: 320 - RNA, 20 nt, synthetic construct
  misc_feature 1..20: note = Synthetic
  source 1..20: mol_type = other RNA
tcggtaagttctgtccaagc

SEQ ID NO: 321 - RNA, 20 nt, synthetic construct
  misc_feature 1..20: note = Synthetic
  source 1..20: mol_type = other RNA
cggtaagttctgtccaagcc

SEQ ID NO: 322 - RNA, 20 nt, synthetic construct
  misc_feature 1..20: note = Synthetic
  source 1..20: mol_type = other RNA
cggtaagttctgtccaagcc

SEQ ID NO: 323 - RNA, 20 nt, synthetic construct
  misc_feature 1..20: note = Synthetic
  source 1..20: mol_type = other RNA
gtaagttctgtccaagcccg

SEQ ID NO: 324 - RNA, 20 nt, synthetic construct
  misc_feature 1..20: note = Synthetic
  source 1..20: mol_type = other RNA
taagttctgtccaagcccgg

SEQ ID NO: 325 - RNA, 20 nt, synthetic construct
  misc_feature 1..20: note = Synthetic
  source 1..20: mol_type = other RNA
aagttctgtccaagcccggt

SEQ ID NO: 326 - RNA, 20 nt, synthetic construct
  misc_feature 1..20: note = Synthetic
  source 1..20: mol_type = other RNA
agttctgtccaagcccggtt

SEQ ID NO: 327 - RNA, 20 nt, synthetic construct
  misc_feature 1..20: note = Synthetic
  source 1..20: mol_type = other RNA
agttctgtccaagcccggtt

SEQ ID NO: 328 - RNA, 20 nt, synthetic construct
  misc_feature 1..20: note = Synthetic
  source 1..20: mol_type = other RNA
gttctgtccaagcccggttg

SEQ ID NO: 329 - RNA, 20 nt, synthetic construct
  misc_feature 1..20: note = Synthetic
  source 1..20: mol_type = other RNA
ttctgtccaagcccggttga

SEQ ID NO: 330 - RNA, 20 nt, synthetic construct
  misc_feature 1..20: note = Synthetic
  source 1..20: mol_type = other RNA
tctgtccaagcccggttgaa

SEQ ID NO: 331 - RNA, 20 nt, synthetic construct
  misc_feature 1..20: note = Synthetic
  source 1..20: mol_type = other RNA
ctgtccaagcccggttgaaa

SEQ ID NO: 332 - RNA, 20 nt, synthetic construct
  misc_feature 1..20: note = Synthetic
  source 1..20: mol_type = other RNA
tgtccaagcccggttgaaat

SEQ ID NO: 333 - RNA, 20 nt, synthetic construct
  misc_feature 1..20: note = Synthetic
  source 1..20: mol_type = other RNA
gtccaagcccggttgaaatc

SEQ ID NO: 334 - RNA, 20 nt, synthetic construct
  misc_feature 1..20: note = Synthetic
  source 1..20: mol_type = other RNA
tccaagcccggttgaaatct

SEQ ID NO: 335 - RNA, 20 nt, synthetic construct
  misc_feature 1..20: note = Synthetic
  source 1..20: mol_type = other RNA
ccaagcccggttgaaatctg

SEQ ID NO: 336 - RNA, 20 nt, synthetic construct
  misc_feature 1..20: note = Synthetic
  source 1..20: mol_type = other RNA
caagcccggttgaaatctgc

SEQ ID NO: 337 - RNA, 20 nt, synthetic construct
  misc_feature 1..20: note = Synthetic
  source 1..20: mol_type = other RNA
aagcccggttgaaatctgcc

SEQ ID NO: 338 - RNA, 20 nt, synthetic construct
  misc_feature 1..20: note = Synthetic
  source 1..20: mol_type = other RNA
agcccggttgaaatctgcca

SEQ ID NO: 339 - RNA, 20 nt, synthetic construct
  misc_feature 1..20: note = Synthetic
  source 1..20: mol_type = other RNA
gcccggttgaaatctgccag

SEQ ID NO: 340 - RNA, 20 nt, synthetic construct
  misc_feature 1..20: note = Synthetic
  source 1..20: mol_type = other RNA
cccggttgaaatctgccaga

SEQ ID NO: 341 - RNA, 20 nt, synthetic construct
  misc_feature 1..20: note = Synthetic
  source 1..20: mol_type = other RNA
ccggttgaaatctgccagag

SEQ ID NO: 342 - RNA, 20 nt, synthetic construct
  misc_feature 1..20: note = Synthetic
  source 1..20: mol_type = other RNA
cggttgaaatctgccagagc

SEQ ID NO: 343 - RNA, 20 nt, synthetic construct
  misc_feature 1..20: note = Synthetic
  source 1..20: mol_type = other RNA
ggttgaaatctgccagagca

SEQ ID NO: 344 - RNA, 20 nt, synthetic construct
  misc_feature 1..20: note = Synthetic
  source 1..20: mol_type = other RNA
gttgaaatctgccagagcag

SEQ ID NO: 345 - RNA, 20 nt, synthetic construct
  misc_feature 1..20: note = Synthetic
  source 1..20: mol_type = other RNA
ttgaaatctgccagagcagg

SEQ ID NO: 346 - RNA, 20 nt, synthetic construct
  misc_feature 1..20: note = Synthetic
  source 1..20: mol_type = other RNA
gaaatctgccagagcaggta

SEQ ID NO: 347 - RNA, 20 nt, synthetic construct
  misc_feature 1..20: note = Synthetic
  source 1..20: mol_type = other RNA
aaatctgccagagcaggtac

SEQ ID NO: 348 - RNA, 20 nt, synthetic construct
  misc_feature 1..20: note = Synthetic
  source 1..20: mol_type = other RNA
atctgccagagcaggtacct

SEQ ID NO: 349 - RNA, 20 nt, synthetic construct
  misc_feature 1..20: note = Synthetic
  source 1..20: mol_type = other RNA
tctgccagagcaggtacctc

SEQ ID NO: 350 - RNA, 20 nt, synthetic construct
  misc_feature 1..20: note = Synthetic
  source 1..20: mol_type = other RNA
tgccagagcaggtacctcca

SEQ ID NO: 351 - RNA, 20 nt, synthetic construct
  misc_feature 1..20: note = Synthetic
  source 1..20: mol_type = other RNA
gccagagcaggtacctccaa

SEQ ID NO: 352 - RNA, 20 nt, synthetic construct
  misc_feature 1..20: note = Synthetic
  source 1..20: mol_type = other RNA
cagagcaggtacctccaaca

SEQ ID NO: 353 - RNA, 20 nt, synthetic construct
  misc_feature 1..20: note = Synthetic
  source 1..20: mol_type = other RNA
agagcaggtacctccaacat

SEQ ID NO: 354 - RNA, 20 nt, synthetic construct
  misc_feature 1..20: note = Synthetic
  source 1..20: mol_type = other RNA
agcaggtacctccaacatca

SEQ ID NO: 355 - RNA, 20 nt, synthetic construct
  misc_feature 1..20: note = Synthetic
  source 1..20: mol_type = other RNA
gcaggtacctccaacatcaa

SEQ ID NO: 356 - RNA, 20 nt, synthetic construct
  misc_feature 1..20: note = Synthetic
  source 1..20: mol_type = other RNA
tcaaggaagatggcatttct

SEQ ID NO: 357 - RNA, 20 nt, synthetic construct
  misc_feature 1..20: note = Synthetic
  source 1..20: mol_type = other RNA
caaggtcacccaccatcacc

SEQ ID NO: 358 - RNA, 20 nt, synthetic construct
  misc_feature 1..20: note = Synthetic
  source 1..20: mol_type = other RNA
gtcacccaccatcaccctct

SEQ ID NO: 359 - RNA, 20 nt, synthetic construct
  misc_feature 1..20: note = Synthetic
  source 1..20: mol_type = other RNA
tcacccaccatcaccctctg

SEQ ID NO: 360 - RNA, 20 nt, synthetic construct
  misc_feature 1..20: note = Synthetic
  source 1..20: mol_type = other RNA
cacccaccatcaccctctgt

SEQ ID NO: 361 - RNA, 20 nt, synthetic construct
  misc_feature 1..20: note = Synthetic
  source 1..20: mol_type = other RNA
gatcaagcagagaaagccag

SEQ ID NO: 362 - RNA, 20 nt, synthetic construct
  misc_feature 1..20: note = Synthetic
  source 1..20: mol_type = other RNA
caagcagagaaagccagtcg

SEQ ID NO: 363 - RNA, 20 nt, synthetic construct
  misc_feature 1..20: note = Synthetic
  source 1..20: mol_type = other RNA
aagccagtcggtaagttctg

SEQ ID NO: 364 - RNA, 20 nt, synthetic construct
  misc_feature 1..20: note = Synthetic
  source 1..20: mol_type = other RNA
agtcggtaagttctgtccaa

SEQ ID NO: 365 - RNA, 20 nt, synthetic construct
  misc_feature 1..20: note = Synthetic
  source 1..20: mol_type = other RNA
gtcggtaagttctgtccaag

SEQ ID NO: 366 - RNA, 20 nt, synthetic construct
  misc_feature 1..20: note = Synthetic
  source 1..20: mol_type = other RNA
tcggtaagttctgtccaagc

SEQ ID NO: 367 - RNA, 20 nt, synthetic construct
  misc_feature 1..20: note = Synthetic
  source 1..20: mol_type = other RNA
cggtaagttctgtccaagcc

SEQ ID NO: 368 - RNA, 20 nt, synthetic construct
  misc_feature 1..20: note = Synthetic
  source 1..20: mol_type = other RNA
ggtaagttctgtccaagccc

SEQ ID NO: 369 - RNA, 20 nt, synthetic construct
  misc_feature 1..20: note = Synthetic
  source 1..20: mol_type = other RNA
gtaagttctgtccaagcccg

SEQ ID NO: 370 - RNA, 20 nt, synthetic construct
  misc_feature 1..20: note = Synthetic
  source 1..20: mol_type = other RNA
taagttctgtccaagcccgg

SEQ ID NO: 371 - RNA, 20 nt, synthetic construct
  misc_feature 1..20: note = Synthetic
  source 1..20: mol_type = other RNA
aagttctgtccaagcccggt

SEQ ID NO: 372 - RNA, 20 nt, synthetic construct
  misc_feature 1..20: note = Synthetic
  source 1..20: mol_type = other RNA
agttctgtccaagcccggtt

SEQ ID NO: 373 - RNA, 20 nt, synthetic construct
  misc_feature 1..20: note = Synthetic
  source 1..20: mol_type = other RNA
gttctgtccaagcccggttg

SEQ ID NO: 374 - RNA, 20 nt, synthetic construct
  misc_feature 1..20: note = Synthetic
  source 1..20: mol_type = other RNA
ttctgtccaagcccggttga

SEQ ID NO: 375 - RNA, 20 nt, synthetic construct
  misc_feature 1..20: note = Synthetic
  source 1..20: mol_type = other RNA
tctgtccaagcccggttgaa

SEQ ID NO: 376 - RNA, 20 nt, synthetic construct
  misc_feature 1..20: note = Synthetic
  source 1..20: mol_type = other RNA
gtccaagcccggttgaaatc

SEQ ID NO: 377 - RNA, 20 nt, synthetic construct
  misc_feature 1..20: note = Synthetic
  source 1..20: mol_type = other RNA
gcatttctagtttggagatg

SEQ ID NO: 378 - RNA, 20 nt, synthetic construct
  misc_feature 1..20: note = Synthetic
  source 1..20: mol_type = other RNA
ggcagtttccttagtaacca

SEQ ID NO: 379 - RNA, 20 nt, synthetic construct
  misc_feature 1..20: note = Synthetic
  source 1..20: mol_type = other RNA
gcagtttccttagtaaccac

SEQ ID NO: 380 - RNA, 20 nt, synthetic construct
  misc_feature 1..20: note = Synthetic
  source 1..20: mol_type = other RNA
cagtttccttagtaaccaca

SEQ ID NO: 381 - RNA, 20 nt, synthetic construct
  misc_feature 1..20: note = Synthetic
  source 1..20: mol_type = other RNA
agtttccttagtaaccacag

SEQ ID NO: 382 - RNA, 20 nt, synthetic construct
  misc_feature 1..20: note = Synthetic
  source 1..20: mol_type = other RNA
gtttccttagtaaccacagg

SEQ ID NO: 383 - RNA, 20 nt, synthetic construct
  misc_feature 1..20: note = Synthetic
  source 1..20: mol_type = other RNA
tttccttagtaaccacaggt

SEQ ID NO: 384 - RNA, 20 nt, synthetic construct
  misc_feature 1..20: note = Synthetic
  source 1..20: mol_type = other RNA
ttccttagtaaccacaggtt

SEQ ID NO: 385 - RNA, 20 nt, synthetic construct
  misc_feature 1..20: note = Synthetic
  source 1..20: mol_type = other RNA
tccttagtaaccacaggttg

SEQ ID NO: 386 - RNA, 20 nt, synthetic construct
  misc_feature 1..20: note = Synthetic
  source 1..20: mol_type = other RNA
ccttagtaaccacaggttgt

SEQ ID NO: 387 - RNA, 20 nt, synthetic construct
  misc_feature 1..20: note = Synthetic
  source 1..20: mol_type = other RNA
gttgtgtcaccagagtaaca

SEQ ID NO: 388 - RNA, 20 nt, synthetic construct
  misc_feature 1..20: note = Synthetic
  source 1..20: mol_type = other RNA
ttgtgtcaccagagtaacag

SEQ ID NO: 389 - RNA, 20 nt, synthetic construct
  misc_feature 1..20: note = Synthetic
  source 1..20: mol_type = other RNA
tgtgtcaccagagtaacagt

SEQ ID NO: 390 - RNA, 19 nt, synthetic construct
  misc_feature 1..19: note = Synthetic
  source 1..19: mol_type = other RNA
gtttccttagtaaccacag

SEQ ID NO: 391 - RNA, 19 nt, synthetic construct
  misc_feature 1..19: note = Synthetic
  source 1..19: mol_type = other RNA
gtttccttagtaaccacag

SEQ ID NO: 392 - RNA, 20 nt, synthetic construct
  misc_feature 1..20: note = Synthetic
  source 1..20: mol_type = other RNA
agtttccttagtaaccacag

SEQ ID NO: 393 - RNA, 20 nt, synthetic construct
  misc_feature 1..20: note = Synthetic
  source 1..20: mol_type = other RNA
tggcatttctagtttggaga

SEQ ID NO: 394 - RNA, 20 nt, synthetic construct
  misc_feature 1..20: note = Synthetic
  source 1..20: mol_type = other RNA
ggtaagttctgtccaagccc

SEQ ID NO: 395 - RNA, 20 nt, synthetic construct
  misc_feature 1..20: note = Synthetic
  source 1..20: mol_type = other RNA
atggcatttctagtttggag

SEQ ID NO: 396 - RNA, 20 nt, synthetic construct
  misc_feature 1..20: note = Synthetic
  source 1..20: mol_type = other RNA
gcatttctagtttggagatg

SEQ ID NO: 397 - RNA, 20 nt, synthetic construct
  misc_feature 1..20: note = Synthetic
  source 1..20: mol_type = other RNA
cagtttccttagtaaccaca

SEQ ID NO: 398 - RNA, 20 nt, synthetic construct
  misc_feature 1..20: note = Synthetic
  source 1..20: mol_type = other RNA
ttccttagtaaccacaggtt

SEQ ID NO: 399 - RNA, 20 nt, synthetic construct
  misc_feature 1..20: note = Synthetic
  source 1..20: mol_type = other RNA
gtacctccaacatcaaggaa

SEQ ID NO: 400 - RNA, 20 nt, synthetic construct
  misc_feature 1..20: note = Synthetic
  source 1..20: mol_type = other RNA
ggcatttctagtttggagat

SEQ ID NO: 401 - RNA, 20 nt, synthetic construct
  misc_feature 1..20: note = Synthetic
  source 1..20: mol_type = other RNA
tggcagtttccttagtaacc

SEQ ID NO: 402 - DNA, 60 bp, Mus musculus
  source 1..60: mol_type = genomic DNA
gtggttactaaggaaactgtcatctccaaactagaaatgccatcttctttgctgttggag

SEQ ID NO: 403 - DNA, 60 bp, Homo sapiens
  source 1..60: mol_type = genomic DNA
gtggttactaaggaaactgccatctccaaactagaaatgccatcttccttgatgttggag

SEQ ID NO: 404 - RNA, 20 nt, synthetic construct
  misc_feature 1..20: note = Synthetic
  source 1..20: mol_type = other RNA
tcaaggaagatggcatttct

SEQ ID NO: 405 - RNA, 20 nt, synthetic construct
  misc_feature 1..20: note = Synthetic
  source 1..20: mol_type = other RNA
gcaaagaagatggcatttct

SEQ ID NO: 406 - RNA, 20 nt, synthetic construct
  misc_feature 1..20: note = Synthetic
  source 1..20: mol_type = other RNA
tcaaggaagatggcatttct

SEQ ID NO: 407 - RNA, 20 nt, synthetic construct
  misc_feature 1..20: note = Synthetic
  source 1..20: mol_type = other RNA
caacatcaaggaagatggca

SEQ ID NO: 408 - RNA, 20 nt, synthetic construct
  misc_feature 1..20: note = Synthetic
  source 1..20: mol_type = other RNA
gatggcatttctagtttgga

SEQ ID NO: 409 - RNA, 20 nt, synthetic construct
  misc_feature 1..20: note = Synthetic
  source 1..20: mol_type = other RNA
gcagtttccttagtaaccac

SEQ ID NO: 410 - RNA, 20 nt, synthetic construct
  misc_feature 1..20: note = Synthetic
  source 1..20: mol_type = other RNA
ttgtgtcaccagagtaacag

SEQ ID NO: 411 - RNA, 20 nt, synthetic construct
  misc_feature 1..20: note = Synthetic
  source 1..20: mol_type = other RNA
gtacctccaacatcaaggaa

SEQ ID NO: 412 - RNA, 20 nt, synthetic construct
  misc_feature 1..20: note = Synthetic
  source 1..20: mol_type = other RNA
tggcatttctagtttggaga

SEQ ID NO: 413 - RNA, 20 nt, synthetic construct
  misc_feature 1..20: note = Synthetic
  source 1..20: mol_type = other RNA
ggcatttctagtttggagat

SEQ ID NO: 414 - RNA, 20 nt, synthetic construct
  misc_feature 1..20: note = Synthetic
  source 1..20: mol_type = other RNA
tggcagtttccttagtaacc

SEQ ID NO: 415 - RNA, 20 nt, synthetic construct
  misc_feature 1..20: note = Synthetic
  source 1..20: mol_type = other RNA
tggcagtttccttagtaacc

SEQ ID NO: 416 - RNA, 20 nt, synthetic construct
  misc_feature 1..20: note = Synthetic
  source 1..20: mol_type = other RNA
ggtaagttctgtccaagccc

SEQ ID NO: 417 - RNA, 20 nt, synthetic construct
  misc_feature 1..20: note = Synthetic
  source 1..20: mol_type = other RNA
ggtaagttctgtccaagccc

SEQ ID NO: 418 - RNA, 20 nt, synthetic construct
  misc_feature 1..20: note = Synthetic
  source 1..20: mol_type = other RNA
caacatcaaggaagatggca

SEQ ID NO: 419 - RNA, 20 nt, synthetic construct
  misc_feature 1..20: note = Synthetic
  source 1..20: mol_type = other RNA
atggcatttctagtttggag

SEQ ID NO: 420 - RNA, 20 nt, synthetic construct
  misc_feature 1..20: note = Synthetic
  source 1..20: mol_type = other RNA
atggcatttctagtttggag

SEQ ID NO: 421 - RNA, 20 nt, synthetic construct
  misc_feature 1..20: note = Synthetic
  source 1..20: mol_type = other RNA
gcatttctagtttggagatg

SEQ ID NO: 422 - RNA, 20 nt, synthetic construct
  misc_feature 1..20: note = Synthetic
  source 1..20: mol_type = other RNA
cagtttccttagtaaccaca

SEQ ID NO: 423 - RNA, 20 nt, synthetic construct
  misc_feature 1..20: note = Synthetic
  source 1..20: mol_type = other RNA
ttccttagtaaccacaggtt

SEQ ID NO: 424 - RNA, 20 nt, synthetic construct
  misc_feature 1..20: note = Synthetic
  source 1..20: mol_type = other RNA
ggtaagttctgtccaagccc

SEQ ID NO: 425 - RNA, 20 nt, synthetic construct
  misc_feature 1..20: note = Synthetic
  source 1..20: mol_type = other RNA
gtacctccaacatcaaggaa

SEQ ID NO: 426 - RNA, 20 nt, synthetic construct
  misc_feature 1..20: note = Synthetic
  source 1..20: mol_type = other RNA
caacatcaaggaagatggca

SEQ ID NO: 427 - RNA, 20 nt, synthetic construct
  misc_feature 1..20: note = Synthetic
  source 1..20: mol_type = other RNA
atggcatttctagtttggag

SEQ ID NO: 428 - RNA, 20 nt, synthetic construct
  misc_feature 1..20: note = Synthetic
  source 1..20: mol_type = other RNA
tggcatttctagtttggaga

SEQ ID NO: 429 - RNA, 20 nt, synthetic construct
  misc_feature 1..20: note = Synthetic
  source 1..20: mol_type = other RNA
ggcatttctagtttggagat

SEQ ID NO: 430 - RNA, 20 nt, synthetic construct
  misc_feature 1..20: note = Synthetic
  source 1..20: mol_type = other RNA
gcatttctagtttggagatg

SEQ ID NO: 431 - RNA, 20 nt, synthetic construct
  misc_feature 1..20: note = Synthetic
  source 1..20: mol_type = other RNA
tggcagtttccttagtaacc

SEQ ID NO: 432 - RNA, 20 nt, synthetic construct
  misc_feature 1..20: note = Synthetic
  source 1..20: mol_type = other RNA
cagtttccttagtaaccaca

SEQ ID NO: 433 - RNA, 20 nt, synthetic construct
  misc_feature 1..20: note = Synthetic
  source 1..20: mol_type = other RNA
agtttccttagtaaccacag

SEQ ID NO: 434 - RNA, 20 nt, synthetic construct
  misc_feature 1..20: note = Synthetic
  source 1..20: mol_type = other RNA
ttccttagtaaccacaggtt

SEQ ID NO: 435 - RNA, 20 nt, synthetic construct
  misc_feature 1..20: note = Synthetic
  source 1..20: mol_type = other RNA
ttgtgtcaccagagtaacag

SEQ ID NO: 436 - RNA, 20 nt, synthetic construct
  misc_feature 1..20: note = Synthetic
  source 1..20: mol_type = other RNA
caacatcaaggaagatggca

SEQ ID NO: 437 - RNA, 20 nt, synthetic construct
  misc_feature 1..20: note = Synthetic
  source 1..20: mol_type = other RNA
gatggcatttctagtttgga

SEQ ID NO: 438 - RNA, 20 nt, synthetic construct
  misc_feature 1..20: note = Synthetic
  source 1..20: mol_type = other RNA
gcagtttccttagtaaccac

SEQ ID NO: 439 - RNA, 20 nt, synthetic construct
  misc_feature 1..20: note = Synthetic
  source 1..20: mol_type = other RNA
ttgtgtcaccagagtaacag

SEQ ID NO: 440 - RNA, 20 nt, synthetic construct
  misc_feature 1..20: note = Synthetic
  source 1..20: mol_type = other RNA
tcaaggaagatggcatttct